(19) United States
(12) Patent Application Publication
(10) Pub. No.: US 2025/0282866 A1
(43) Pub. Date: Sep. 11, 2025

(54) TREATMENT OF DISEASES RELATED TO ATP-BINDING CASSETTE TRANSPORTER 1 DYSFUNCTION USING TREM2 AGONISTS

(71) Applicant: VIGIL NEUROSCIENCE, INC., Watertown, MA (US)

(72) Inventors: Spyridon PAPAPETROPOULOS, Watertown, MA (US); Richard FISHER, Watertown, MA (US); Matthew BRENNAN, Watertown, MA (US)

(21) Appl. No.: 18/984,651

(22) Filed: Dec. 17, 2024

Related U.S. Application Data
(62) Division of application No. 17/457,727, filed on Dec. 6, 2021, now abandoned.
(60) Provisional application No. 63/121,404, filed on Dec. 4, 2020.

(51) Int. Cl. C07K 16/28; A61K 31/739; A61K 38/17; A61P 25/00

(52) U.S. Cl. CPC C07K 16/2803; A61K 31/739; A61K 38/1709; A61P 25/00; C07K 2317/24; C07K 2317/75

(57) ABSTRACT

The present invention provides a method of treating a disease or disorder caused by and/or associated with ABCD1 dysfunction in a human patient, the method comprising administering to the patient in need thereof an effective amount of a compound that increases the activity of triggering receptor expressed on myeloid cells 2 (TREM2). In some embodiments, compound that increases the activity of TREM2 is an agonist of TREM2. In some embodiments, the agonist of TREM2 is a small molecule agonist of TREM2 or an antibody agonist of TREM2.

### CROSS REFERENCE TO RELATED APPLICATIONS

This application is a divisional of U.S. application Ser. No. 17/457,727, filed Dec. 6, 2021, which claims priority benefit of U.S. Provisional Application No. 63/121,404, filed Dec. 4, 2020, each of which is hereby incorporated by reference in its entirety.

### REFERENCE TO SEQUENCE LISTING

The instant application contains a Sequence Listing which has been submitted electronically in ASCII format and is hereby incorporated by reference in its entirety. Said ASCII copy, created on Dec. 17, 2024, is named 2024-12-17-Sequence-Listing-5258.0070003.txt and is 2,755,362 bytes in size.

### TECHNICAL FIELD OF THE INVENTION

The present invention relates to compounds and methods of use thereof for treating diseases and disorders caused by ATP-binding cassette transporter 1 (ABCD1) dysfunction.

### BACKGROUND OF THE INVENTION

Microglia are brain-resident macrophages with many homeostatic and injury responsive roles, including trophic and phagocytic functions. Microglia are highly dependent on peroxidation for maintaining normal function. The ATP-binding cassette transporter 1 (ABCD1) gene encodes a key peroxisomal protein responsible for transport of activated very long chain fatty acids (VLCFA) into the peroxisome for further degradation and beta-oxidation for energy production. Therefore, mutations in the ABCD1 gene can lead to microglial dysfunction and damage due to accumulation of VLCFA, resulting in neurological and adrenal gland diseases and disorders. X-linked adrenoleukodystrophy (X-ALD) is one such condition associated with ABCD1 mutations, characterized by cerebral and spinal cord white matter degeneration with demyelination and adrenal insufficiency, which lead to progressive cognitive and motor dysfunction and ultimately death. To date, there are no known treatments for diseases and disorders caused by ABCD1 dysfunction, and patients are usually treated by managing the symptoms of the disease. Therefore, there remains a need in the art for methods of treating diseases and disorders caused by ABCD1 loss of function mutations.

### SUMMARY OF THE INVENTION

In one aspect, the present invention provides a method of treating a disease or disorder caused by and/or associated with a dysfunction in ABCD1 in a human patient, the method comprising administering to the patient an effective amount of a compound that increases the activity of triggering receptor expressed on myeloid cells 2 (TREM2). In some embodiments, the compound that increases the activity of TREM2 is an agonist of TREM2. In some embodiments, the agonist of TREM2 is a small molecule agonist of TREM2 or an antibody agonist of TREM2. In some embodiments, the disease or disorder caused by and/or associated with a dysfunction in ABCD1 is x-ALD.

### DETAILED DESCRIPTION OF CERTAIN EMBODIMENTS

### TREM2, ABCD1 and X-ALD

TREM2 is a member of the Ig superfamily of receptors that is expressed on cells of myeloid lineage, including macrophages, dendritic cells, and microglia (Schmid et al., Journal of Neurochemistry, Vol. 83: 1309-1320, 2002; Colonna, Nature Reviews Immunology, Vol. 3: 445-453, 2003; Kiialainen et al., Neurobiology of Disease, 2005, 18: 314-322). TREM2 is an innate immune receptor that binds many endogenous substrates, and signals through a short intracellular domain that complexes with the adaptor protein DAP12, the cytoplasmic domain of which comprises an ITAM motif (Bouchon et al., The Journal of Experimental Medicine, 2001, 194: 1111-1122). Upon activation of TREM2, tyrosine residues within the ITAM motif in DAP12 are phosphorylated by the Src family of kinases, providing docking sites for the tyrosine kinase ξ-chain-associated protein 70 (ZAP70) and spleen tyrosine kinase (Syk) via their SH2 domains (Colonna, Nature Reviews Immunology, 2003, 3:445-453; Ulrich and Holtzman, ACS Chem. Neurosci., 2016, 7:420-427). The ZAP70 and Syk kinases induce activation of several downstream signaling cascades, including phosphatidylinositol 3-kinase (PI3K), protein kinase C (PKC), extracellular regulated kinase (ERK), and elevation of intracellular calcium (Colonna, Nature Reviews Immunology, 2003, 3:445-453; Ulrich and Holtzman, ACS Chem. Neurosci., 2016, 7:420-427). The wild-type human TREM2 amino acid sequence is provided as SEQ ID NO: 1.

TREM2 has been implicated in several myeloid cell processes, including phagocytosis, proliferation, survival, and regulation of inflammatory cytokine production (Ulrich and Holtzman, ACS Chem. Neurosci., 2016, 7: 420-427). One of the key TREM2 functions is regulating myeloid cell number. Knocking down expression of TREM2 in primary microglia using translation blockers leads to reduced cell number (Zheng, et al., Neurobiol. Aging, 2016; 42: 132-141). Evidence suggests that in various contexts, TREM2 is important for myeloid cell survival, proliferation and chemotaxis, all of which could lead to disease-associated increases in myeloid cell number including microglia (Jay, et al., Mol Neurodegener. 2017; 12(1):56).

A well-characterized function of TREM2 is to enhance phagocytosis. TREM2 is expressed in a subset of myeloid cells within the CNS that have high phagocytic capacity (Bisht et al., Glia. 2016; 64: 826-839). Across numerous in vitro studies, loss of TREM2 results in reduced phagocytosis of a variety of substrates, including apoptotic neurons or neuronal cell lines (Takahashi et al., Exp Med. 2005; 201(4):647-657.; Hsieh et al., J Neurochem. 2009; 109(4): 1144-1156). Conversely, TREM2 activation or overexpression enhanced uptake of these substrates (Takahashi et al., J Exp Med. 2005; 201(4):647-657; Takahashi et al., PLoS Med. 2007; 4(4):e124; Jiang et al., Neuropsychopharmacology. 2014; 39(13): 2949-2962.). TREM2 is important for clearance of myelin debris in experimental autoimmune encephalomyelitis (EAE) (Takahashi et al., PLoS Med. 2007; 4(4):e124) and peri-infarct tissue in mice following middle coronary artery occlusion (MCAO) (Kawabori et al., J Neurosci. 2015; 35(8): 3384-3396).

TREM2 has been classically described as being anti-inflammatory and several in vitro and in vivo studies are supportive of an anti-inflammatory role for TREM2 in certain contexts (Yin et al., Traffic. 2016; 17(12): 1286-1296). Knocking down TREM2 in cell lines increases levels of proinflammatory mediators such as iNOS, TNFα, IL1β and ILδ (Yin et al., Traffic. 2016; 17(12): 1286-1296) in response to apoptotic neuronal membrane debris (Takahashi et al., J Exp Med. 2005; 201(4):647-657.), TLR ligands (Turnbull et al., J Immunol. 2006; 177(6):3520-3524.), including LPS (Gawish et al., FASEB J. 2015 Apr; 29(4):1247-1257.; Gao et al., Mol Med Rep. 2013 March; 7(3):921-926.; Takahashi et al., PLoS Med. 2007; 4(4):e124.) and Aβ42 (Jiang et al., Neuropsychopharmacology. 2014; 39(13): 2949-2962.). Moreover, overexpressing TREM2 in cell lines or amyloid (Jiang et al., Neuropsychopharmacology. 2014; 39(13): 2949-2962.) and tau mouse models of AD (Jiang et al., Neuropharmacology. 2016; 105:196-206.) reduced levels of these pro-inflammatory transcripts. Together, these studies suggest that in some contexts, TREM2 can attenuate inflammatory responses.

TREM2 has been linked to several serious diseases. For instance, mutations in both TREM2 and DAP12 have been linked to the autosomal recessive disorder Nasu-Hakola Disease, which is characterized by bone cysts, muscle wasting and demyelination phenotypes (Guerreiro et al., New England Journal of Medicine, 2013, 368: 117-127). Variants in the TREM12 gene have been linked to increased risk for Alzheimer's disease (AD) and other forms of dementia including frontotemporal dementia and amyotrophic lateral sclerosis (Jonsson et al., New England Journal of Medicine, 2013, 368:107-116; Guerreiro et al., JAMA Neurology, 2013, 70:78-84; Jay et al., Journal of Experimental Medicine, 2015, 212: 287-295; Cady et al., JAMA Neurol. 2014 April; 71(4):449-53). Impairment in microgliosis has been reported in animal models of prion disease, multiple sclerosis, and stroke, suggesting that TREM2 may play an important role in supporting microgliosis in response to pathology or damage in the central nervous system (Ulrich and Holtzman, ACS Chem. Neurosci., 2016, 7: 420-427).

The ABCD1 gene provides instructions for producing the adrenoleukodystrophy protein (ALDP). ABCD1 (ALDP) maps to Xq28. ABCD1 is a member of the ATP-binding cassette (ABC) transporter superfamily. The superfamily contains membrane proteins that translocate a wide variety of substrates across extra- and intracellular membranes, including metabolic products, lipids and sterols, and drugs. ALDP is located in the membranes of cell structures called peroxisomes. Peroxisomes are small sacs within cells that process many types of molecules. ALDP brings a group of fats called very long-chain fatty acids (VLCFAs) into peroxisomes, where they are broken down. As ABCD1 is highly expressed in microglia, it is possible that microglial dysfunction and their close interaction with other cell types actively participates in neurodegenerative processes (Gong et al., Annals of Neurology. 2017; 82(5):813-827.). It has been shown that severe microglia loss and damage is an early feature in patients with cerebral form of X-linked ALD (cALD) carrying ABCD1 mutations (Bergner et al., Glia. 2019; 67: 1196-1209). It has also been shown that ABCD1-deficiency leads to an impaired plasticity of myeloid lineage cells that is reflected in incomplete establishment of anti-inflammatory responses, thus possibly contributing to the devastating rapidly progressive demyelination in cerebral adrenoleukodystrophy (Weinhor et al., BRAIN 2018: 141; 2329-2342). It has also been shown in a recent report of 83 young males with cALD that patients harboring the APOE4 allele, a known ligand of TREM2, have an increased burden of cerebral disease involvement as determined by Loes score, gadolinium intensity score (GIS), and neurologic function score (NFS) (Orchard et al., Nature Scientific Reports 2019 9:7858). These findings emphasize microglia/monocytes/macrophages as crucial therapeutic targets for preventing or stopping myelin destruction in patients with X-linked adrenoleukodystrophy.

The present invention relates to the unexpected discovery that administration of a TREM2 agonist can rescue the loss of microglia in cells having mutations in the ABCD1 gene. It has been previously shown that TREM2 agonist antibody 4D9 increases ATP luminescence (a measure of cell number and activity) in a dose dependent manner when the levels of M-CSF in media are reduced to 5 ng/mL (Schlepckow et al, EMBO Mol Med., 2020) and that TREM2 agonist AL002c increases ATP luminescence when M-CSF is completely removed from the media (Wang et al, J. Exp. Med.; 2020, 217(9): e20200785). This finding suggests that TREM2 agonism can compensate for deficiency in ABCD1 function leading to sustained activation, proliferation, chemotaxis of microglia, maintenance of anti-inflammatory environment and reduced astrocytosis caused by a decrease in ABCD1 and accumulation of VLCFAs. The present invention relates to the unexpected discovery that activation of TREM2 can rescue microglia harboring the ABCD1 mutation and challenged with an increase in VLCFA, and that this effect may be also observed in patients suffering from loss of functional microglia due to ABCD1 mutation. This discovery has not been previously taught or suggested in the available art.

To date, no prior study has shown that TREM2 agonism can rescue the loss of microglia in cells where mutations in the ABCD1 and a VLCFA increase is present. No prior study has taught or suggested that reversal of the loss of microglia due to an ABCD1 mutation through TREM2 agonism can be used to treat a disease or disorder caused by and/or associated with an ABCD1 mutation.

X-linked adrenoleukodystrophy (x-ALD) is an X chromosome-linked central nervous system disease caused by an ABCD1 mutation that manifests in the form of variable developmental behavioral, cognitive, motor and sensory function changes in patients suffering from the disease. Three main phenotypes are seen in affected males: (1) The childhood cerebral form manifests most commonly between ages four and eight years. It initially resembles attention-deficit disorder or hyperactivity; progressive impairment of cognition, behavior, vision, hearing, and motor function follow the initial symptoms and often lead to total disability within six months to two years and death within 5 years. (2) Adrenomyeloneuropathy (AMN) manifests most commonly between the second and fourth decades as progressive stiffness and weakness of the legs, sphincter disturbances, sexual dysfunction, and often, impaired adrenocortical function; all symptoms are progressive over decades. (3) “Addison disease only” presents with primary adrenocortical insufficiency between age two years and adulthood and most commonly by age 7.5 years, without evidence of neurologic abnormality. The cerebral childhood form of x-ALD also know as cerebral ALD (cALD) is characterized by patchy cerebral white matter abnormalities visible by magnetic resonance imaging. However, the clinical symptoms and MRI changes are not specific to cALD and are common for other neurological conditions, including Adult-onset leukoencephalopathy with axonal spheroids and pigmented glia (ALSP), Nasu-Hakola disease (NHD) and other leukodystrophies, making diagnosis and treatment of cALD very difficult.

Studies have discovered that x-ALD is a genetic disorder in which male patients that carry a loss of function mutation in the peroxisomal transporter gene ABCD1, leading to VLCFA increase and activation of inflammatory processes leading to demyelination and axonal degeneration. In one aspect, the present invention relates to the surprising discovery that activation of the TREM2 pathway can rescue the loss of microglia in patients carrying ABCD1 mutations, preventing microglia apoptosis, thereby treating ABCD1-related conditions, such as, but not limited to, x-ALD.

The present invention also relates to the surprising discovery that neurofilament light chain and neurofilament heavy chain proteins can serve as a therapeutic biomarker to determine treatment efficacy in patients suffering from a disease or disorder caused by and/or associated with a ABCD1 dysfunction, such as x-ALD. Neurofilament light chain (NfL) is highly elevated in the plasma, serum and CSF of patients with x-ALD, (van Ballegoij, et al., Ann Clin Transl Neurol, 7: 2127-2136.). cALD is characterized by severe and rapid myelin breakdown followed by neurodegeneration. Mice exposed to cuprizone, a model of acute demyelination, show elevations in plasma NfL (Taylor Meadows et al, European Charcot Foundation 25th Annual Meeting; November 30-Dec. 2, 2017; Baveno, Italy). Additionally, TREM2 knockout mice exposed to cuprizone show increased neurotoxicity and further increases in plasma and CSF NfL (Nugent et al, Neuron; 2020, 105(5): 837-854; O'Loughlin et al, Poster #694 ADPD Symposium, Lisbon Portugal, April 2019.). Patients with cALD have quantitatively fewer microglia than healthy individuals in multiple regions of the brain (Bergner et al., Glia. 2019; 67(6):1196-1209). The present invention relates to the unexpected discovery that neurofilament is broken down in the neurons of animals suffering from a disease or disorder caused by and/or associated with a ABCD1 dysfunction, such as x-ALD, resulting in an increase in neurofilament breakdown products in the plasma, serum and cerebral spinal fluid (CSF), and that efficacy of treatment of the dieasese or disorder with a TREM2 agonist can be determined by measuring central levels of neurofilament and central nervous system (CNS), plasma and serum levels of its degradation products, namely neurofilament light chain and neurofilament heavy chain proteins. In one aspect, the present invention provides methods for selecting x-ALD patients that are likely to experience progression of their neurodegenerative or other disease phenotypes based on neurofilament light chain or neurofilament heavy chain levels, thereby informing the timing of treatment with a TREM2 agonist.

### Definitions

Unless defined otherwise, all technical and scientific terms used herein have the same meaning as commonly understood by one of ordinary skill in the art. Accordingly, the following terms are intended to have the following meanings.

“Agonist” or an “activating” agent, such as a compound or antibody, is an agent that induces (e.g., increases) one or more activities or functions of the target (e.g., TREM2) of the agent after the agent binds the target.

“Antagonist” or a “blocking” agent, such as a compound or antibody, is an agent that reduces or eliminates (e.g., decreases) binding of the target to one or more ligands after the agent binds the target, and/or that reduces or eliminates (e.g., decreases) one or more activities or functions of the target after the agent binds the target. In some embodiments, antagonist agent, or blocking agent substantially or completely inhibits target binding to one or more of its ligand and/or one or more activities or functions of the target.

“Antibody” is used in the broadest sense and refers to an immunoglobulin or fragment thereof, and encompasses any such polypeptide comprising an antigen-binding fragment or region of an antibody. The recognized immunoglobulin genes include the kappa, lambda, alpha, gamma, delta, epsilon and mu constant region genes, as well as myriad immunoglobulin variable region genes. Light chains are generally classified as either kappa or lambda. Heavy chains are classified as gamma, mu, alpha, delta, or epsilon, which in turn define the immunoglobulin classes, IgG, IgM, IgA, IgD and IgE, respectively. Immunoglobulin classes may also be further classified into subclasses, including IgG subclasses IgG₁, IgG₂, IgG₃, and IgG₄; and IgA subclasses IgA₁ and IgA₂. The term includes, but is not limited to, polyclonal, monoclonal, monospecific, multispecific (e.g., bispecific antibodies), natural, humanized, human, chimeric, synthetic, recombinant, hybrid, mutated, grafted, antibody fragments (e.g., a portion of a full-length antibody, generally the antigen binding or variable region thereof, e.g., Fab, Fab′, F(ab′)2, and Fv fragments), and in vitro generated antibodies so long as they exhibit the desired biological activity. The term also includes single chain antibodies, e.g., single chain Fv (sFv or scFv) antibodies, in which a variable heavy and a variable light chain are joined together (directly or through a peptide linker) to form a continuous polypeptide.

“Isolated” refers to a change from a natural state, that is, changed and/or removed from its original environment. For example, a polynucleotide or polypeptide (e.g., an antibody) is isolated when it is separated from material with which it is naturally associated in the natural environment. Thus, an “isolated antibody” is one which has been separated and/or recovered from a component of its natural environment.

“Purified antibody” refers to an antibody preparation in which the antibody is at least 80% or greater, at least 85% or greater, at least 90% or greater, at least 95% or greater by weight as compared to other contaminants (e.g., other proteins) in the preparation, such as by determination using SDS-polyacrylamide gel electrophoresis (PAGE) or capillary electrophoresis- (CE) SDS under reducing or non-reducing conditions.

“Extracellular domain” and “ectodomain” are used interchangeably when used in reference to a membrane bound protein and refer to the portion of the protein that is exposed on the extracellular side of a lipid membrane of a cell.

“Binds specifically” in the context of any binding agent, e.g., an antibody, refers to a binding agent that binds specifically to an antigen or epitope, such as with a high affinity, and does not significantly bind other unrelated antigens or epitopes.

“Functional” refers to a form of a molecule which possesses either the native biological activity of the naturally existing molecule of its type, or any specific desired activity, for example as judged by its ability to bind to ligand molecules. Examples of “functional” polypeptides include an antibody binding specifically to an antigen through its antigen-binding region.

“Antigen” refers to a substance, such as, without limitation, a particular peptide, protein, nucleic acid, or carbohydrate which can bind to a specific antibody.

“Epitope” or “antigenic determinant” refers to that portion of an antigen capable of being recognized and specifically bound by a particular antibody. When the antigen is a polypeptide, epitopes can be formed from contiguous amino acids and/or noncontiguous amino acids juxtaposed by tertiary folding of a protein. Linear epitope is an epitope formed from contiguous amino acids on the linear sequence of amino acids. A linear epitope may be retained upon protein denaturing. Conformational or structural epitope is an epitope composed of amino acid residues that are not contiguous and thus comprised of separated parts of the linear sequence of amino acids that are brought into proximity to one another by folding of the molecule, such as through secondary, tertiary, and/or quaternary structures. A conformational or structural epitope may be lost upon protein denaturation. In some embodiments, an epitope can comprise at least 3, and more usually, at least 5 or 8-10 amino acids in a unique spatial conformation. Thus, an epitope as used herein encompasses a defined epitope in which an antibody binds only portions of the defined epitope. There are many methods known in the art for mapping and characterizing the location of epitopes on proteins, including solving the crystal structure of an antibody-antigen complex, competition assays, gene fragment expression assays, mutation assays, and synthetic peptide-based assays, as described, for example, in Using Antibodies: A Laboratory Manual, Chapter 11, Harlow and Lane, eds., Cold Spring Harbor Laboratory Press, Cold Spring Harbor, New York (1999).

“Protein,” “polypeptide,” or “peptide” denotes a polymer of at least two amino acids covalently linked by an amide bond, regardless of length or post-translational modification (e.g., glycosylation, phosphorylation, lipidation, myristoylation, ubiquitination, etc.). Included within this definition are D- and L-amino acids, and mixtures of D- and L-amino acids. Unless specified otherwise, the amino acid sequences of a protein, polypeptide, or peptide are displayed herein in the conventional N-terminal to C-terminal orientation.

“Polynucleotide” and “nucleic acid” are used interchangeably herein and refer to two or more nucleosides that are covalently linked together. The polynucleotide may be wholly comprised of ribonucleosides (i.e., an RNA), wholly comprised of 2′ deoxyribonucleotides (i.e., a DNA) or mixtures of ribo- and 2′ deoxyribonucleosides. The nucleosides will typically be linked together by sugar-phosphate linkages (sugar-phosphate backbone), but the polynucleotides may include one or more non-standard linkages. Non-limiting example of such non-standard linkages include phosphoramidates, phosphorothioates, and amides (see, e.g., Eckstein, F., Oligonucleotides and Analogues: A Practical Approach, Oxford University Press (1992)).

“Operably linked” or “operably associated” refers to a situation in which two or more polynucleotide sequences are positioned to permit their ordinary functionality. For example, a promoter is operably linked to a coding sequence if it is capable of controlling the expression of the sequence. Other control sequences, such as enhancers, ribosome binding or entry sites, termination signals, polyadenylation sequences, and signal sequences are also operably linked to permit their proper function in transcription or translation.

“Amino acid position” and “amino acid residue” are used interchangeably to refer to the position of an amino acid in a polypeptide chain. In some embodiments, the amino acid residue can be represented as “XN”, where X represents the amino acid and the N represents its position in the polypeptide chain. Where two or more variations, e.g., polymorphisms, occur at the same amino acid position, the variations can be represented with a “/” separating the variations. A substitution of one amino acid residue with another amino acid residue at a specified residue position can be represented by XNY, where X represents the original amino acid, N represents the position in the polypeptide chain, and Y represents the replacement or substitute amino acid. When the terms are used to describe a polypeptide or peptide portion in reference to a larger polypeptide or protein, the first number referenced describes the position where the polypeptide or peptide begins (i.e., amino end) and the second referenced number describes where the polypeptide or peptide ends (i.e., carboxy end).

“Polyclonal” antibody refers to a composition of different antibody molecules which is capable of binding to or reacting with several different specific antigenic determinants on the same or on different antigens. A polyclonal antibody can also be considered to be a “cocktail of monoclonal antibodies.” The polyclonal antibodies may be of any origin, e.g., chimeric, humanized, or fully human.

“Monoclonal antibody” refers to an antibody obtained from a population of substantially homogeneous antibodies, i.e., the individual antibodies comprising the population are identical except for possible naturally occurring mutations that may be present in minor amounts. Each monoclonal antibody is directed against a single determinant on the antigen. In some embodiments, monoclonal antibodies to be used in accordance with the present disclosure can be made by the hybridoma method described by Kohler et al., 1975, Nature 256:495-7, or by recombinant DNA methods. The monoclonal antibodies can also be isolated, e.g., from phage antibody libraries.

“Chimeric antibody” refers to an antibody made up of components from at least two different sources. A chimeric antibody can comprise a portion of an antibody derived from a first species fused to another molecule, e.g., a portion of an antibody derived from a second species. In some embodiments, a chimeric antibody comprises a portion of an antibody derived from a non-human animal, e.g., mouse or rat, fused to a portion of an antibody derived from a human. In some embodiments, a chimeric antibody comprises all or a portion of a variable region of an antibody derived from a non-human animal fused to a constant region of an antibody derived from a human.

“Humanized antibody” refers to an antibody that comprises a donor antibody binding specificity, e.g., the CDR regions of a donor antibody, such as a mouse monoclonal antibody, grafted onto human framework sequences. A “humanized antibody” typically binds to the same epitope as the donor antibody.

“Fully human antibody” or “human antibody” refers to an antibody that comprises human immunoglobulin protein sequences only. A fully human antibody may contain murine carbohydrate chains if produced in a non-human cell, e.g., a mouse, in a mouse cell, or in a hybridoma derived from a mouse cell.

“Full-length antibody,” “intact antibody” or “whole antibody” are used interchangeably to refer to an antibody, such as an anti-TREM2 antibody of the present disclosure, in its substantially intact form, as opposed to an antibody fragment. Specifically whole antibodies include those with heavy and light chains including an Fc region. The constant domains may be native sequence constant domains {e.g., human native sequence constant domains) or amino acid sequence variants thereof. In some cases, the intact antibody may have one or more effector functions.

“Antibody fragment” or “antigen-binding moiety” refers to a portion of a full length antibody, generally the antigen binding or variable domain thereof. Examples of antibody fragments include Fab, Fab′, F(ab′)2, and Fv fragments; diabodies; linear antibodies; single-chain antibodies; and multispecific antibodies formed from antibody fragments that bind two or more different antigens. Several examples of antibody fragments containing increased binding stoichiometries or variable valencies (2, 3 or 4) include triabodies, trivalent antibodies and trimerbodies, tetrabodies, tandAbs®, di-diabodies and (sc(Fv)2)₂ molecules, and all can be used as binding agents to bind with high affinity and avidity to soluble antigens (see, e.g., Cuesta et al., 2010, Trends Biotech. 28:355-62).

“Single-chain Fv” or “sFv” antibody fragment comprises the VH and VL domains of an antibody, where these domains are present in a single polypeptide chain. Generally, the Fv polypeptide further comprises a polypeptide linker between the VH and VL domains which enables the sFv to form the desired structure for antigen binding. For a review of sFv, see Pluckthun in The Pharmacology of Monoclonal Antibodies, Vol. 113, pp. 269-315, Rosenberg and Moore, eds., Springer-Verlag, New York (1994).

“Diabodies” refers to small antibody fragments with two antigen-binding sites, which comprise a heavy chain variable domain (VH) connected to a light chain variable domain (VL) in the same polypeptide chain (VH-VL). By using a linker that is short to allow pairing between the two domains on the same chain, the domains are forced to pair with the complementary domains of another chain and create two antigen-binding sites.

“Antigen binding domain” or “antigen binding portion” refers to the region or part of the antigen binding molecule that specifically binds to and complementary to part or all of an antigen. In some embodiments, an antigen binding domain may only bind to a particular part of the antigen (e.g., an epitope), particularly where the antigen is large. An antigen binding domain may comprise one or more antibody variable regions, particularly an antibody light chain variable region (VL) and an antibody heavy chain variable region (VH), and particularly the complementarity determining regions (CDRs) on each of the VH and VL chains.

“Variable region” and “variable domain” are used interchangeably to refer to the polypeptide region that confers the binding and specificity characteristics of each particular antibody. The variable region in the heavy chain of an antibody is referred to as “VH” while the variable region in the light chain of an antibody is referred to as “VL”. The major variability in sequence is generally localized in three regions of the variable domain, denoted as “hypervariable regions” or “CDRs” in each of the VL region and VH region, and forms the antigen binding site. The more conserved portions of the variable domains are referred to as the framework region FR.

“Complementarity-determining region” and “CDR” are used interchangeably to refer to non-contiguous antigen binding regions found within the variable region of the heavy and light chain polypeptides of an antibody molecule. In some embodiments, the CDRs are also described as “hypervariable regions” or “HVR”. Generally, naturally occurring antibodies comprise six CDRs, three in the VH (referred to as: CDR H1 or H1; CDR H2 or H2; and CDR H3 or H3) and three in the VL (referred to as: CDR L1 or L1; CDR L2 or L2; and CDR L3 or L3). The CDR domains have been delineated using various approaches, and it is to be understood that CDRs defined by the different approaches are to be encompassed herein. The “Kabat” approach for defining CDRs uses sequence variability and is the most commonly used (Kabat et al., 1991, “Sequences of Proteins of Immunological Interest, 5^{th} Ed.” NIH 1:688-96). “Chothia” uses the location of structural loops (Chothia and Lesk, 1987, J Mol Biol. 196:901-17). CDRs defined by “AbM” are a compromise between the Kabat and Chothia approach, and can be delineated using Oxford Molecular AbM antibody modeling software (see, Martin et al., 1989, Proc. Natl Acad Sci USA. 86:9268; see also, world wide web www.bioinf-org.uk/abs). The “Contact” CDR delineations are based on analysis of known antibody-antigen crystal structures (see, e.g., MacCallum et al., 1996, J. Mol. Biol. 262, 732-45). The CDRs delineated by these methods typically include overlapping or subsets of amino acid residues when compared to each other.

It is to be understood that the exact residue numbers which encompass a particular CDR will vary depending on the sequence and size of the CDR, and those skilled in the art can routinely determine which residues comprise a particular CDR given the amino acid sequence of the variable region of an antibody.

Kabat, supra, also defined a numbering system for variable domain sequences that is applicable to any antibody. The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU or, Kabat numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. References to residue numbers in the variable domain of antibodies means residue numbering by the Kabat numbering system. References to residue numbers in the constant domain of antibodies means residue numbering by the EU or, Kabat numbering system {e.g., see United States Patent Publication No. 2010-280227). One of skill in the art can assign this system of “Kabat numbering” to any variable domain sequence. Accordingly, unless otherwise specified, references to the number of specific amino acid residues in an antibody or antigen binding fragment are according to the Kabat numbering system.

“Framework region” or “FR region” refers to amino acid residues that are part of the variable region but are not part of the CDRs (e.g., using the Kabat, Chothia or AbM definition). The variable region of an antibody generally contains four FR regions: FR1, FR2, FR3 and FR4. Accordingly, the FR regions in a VL region appear in the following sequence: FR_{L}1-CDR L1-FR_{L}2-CDR L2-FR_{L}3-CDR L3-FR_{L}4, while the FR regions in a VH region appear in the following sequence: FR1_{H}-CDR H1-FR_{H}2-CDR H2-FR_{H}3-CDR H3-FR_{H}4.

“Constant region” or “constant domain” refers to a region of an immunoglobulin light chain or heavy chain that is distinct from the variable region. The constant domain of the heavy chain generally comprises at least one of: a CH1 domain, a Hinge (e.g., upper, middle, and/or lower hinge region), a CH2 domain, and a CH3 domain. In some embodiments, the antibody can have additional constant domains CH4 and/or CH5. In some embodiments, an antibody described herein comprises a polypeptide containing a CH1 domain; a polypeptide comprising a CH1 domain, at least a portion of a Hinge domain, and a CH2 domain; a polypeptide comprising a CH1 domain and a CH3 domain; a polypeptide comprising a CH1 domain, at least a portion of a Hinge domain, and a CH3 domain, or a polypeptide comprising a CH1 domain, at least a portion of a Hinge domain, a CH2 domain, and a CH3 domain. In some embodiments, the antibody comprises a polypeptide which includes a CH3 domain. The constant domain of a light chain is referred to a CL, and in some embodiments, can be a kappa or lambda constant region. However, it will be understood by one of ordinary skill in the art that these constant domains (e.g., the heavy chain or light chain) may be modified such that they vary in amino acid sequence from the naturally occurring immunoglobulin molecule.

“Fc region” or “Fc portion” refers to the C terminal region of an immunoglobulin heavy chain. The Fc region can be a native-sequence Fc region or a non-naturally occurring variant Fc region. Generally, the Fc region of an immunoglobulin comprises constant domains CH2 and CH3. Although the boundaries of the Fc region can vary, in some embodiments, the human IgG heavy chain Fc region can be defined to extend from an amino acid residue at position C226 or from P230 to the carboxy terminus thereof. In some embodiments, the “CH2 domain” of a human IgG Fc region, also denoted as “Cγ2”, generally extends from about amino acid residue 231 to about amino acid residue 340. In some embodiments, N-linked carbohydrate chains can be interposed between the two CH2 domains of an intact native IgG molecule. In some embodiments, the CH3 domain” of a human IgG Fc region comprises residues C-terminal to the CH2 domain, e.g., from about amino acid residue 341 to about amino acid residue 447 of the Fc region. A “functional Fc region” possesses an “effector function” of a native sequence Fc region. Exemplary Fc “effector functions” include, among others, C1q binding; complement dependent cytotoxicity (CDC); Fc receptor binding; antibody dependent cell-mediated cytotoxicity (ADCC); phagocytosis; down regulation of cell-surface receptors (e.g., LT receptor); etc. Such effector functions generally require the Fc region to be combined with a binding domain (e.g., an antibody variable domain) and can be assessed using various assays known in the art.

“Native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

“Variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Affinity-matured” antibody, such as an affinity matured anti-TREM2 antibody of the present disclosure, is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology, 1992, 10:779-783 describes affinity maturation by VH- and VL-domain shuffling. Random mutagenesis of HVR and/or framework residues is described by, for example: Barbas et al., Proc Nat. Acad. Sci. USA., 1994, 91:3809-3813; Schier et al. Gene, 1995, 169: 147-155; Yelton et al., Immunol., 1995, 155: 1994-2004; Jackson et al., Immunol., 1995, 154(7):3310-9; and Hawkins et al, J. Mol. Biol., 1992, 226:889-896.

“Binding affinity” refers to strength of the sum total of noncovalent interactions between a ligand and its binding partner. In some embodiments, binding affinity is the intrinsic affinity reflecting a one-to-one interaction between the ligand and binding partner. The affinity is generally expressed in terms of equilibrium association (K_{A}) or dissociation constant (K_{D}), which are in turn reciprocal ratios of dissociation (k_{off}) and association rate constants (kₒₙ).

“Percent (%) sequence identity” and “percentage sequence homology” are used interchangeably herein to refer to comparisons among polynucleotides or polypeptides, and are determined by comparing two optimally aligned sequences over a comparison window, wherein the portion of the polynucleotide or polypeptide sequence in the comparison window may comprise gaps as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv Appl Math. 2:482, by the homology alignment algorithm of Needleman and Wunsch, 1970, J Mol Biol. 48:443, by the search for similarity method of Pearson and Lipman, 1988, Proc Natl Acad Sci USA. 85:2444-8, and particularly by computerized implementations of these algorithms (e.g., BLAST, ALIGN, GAP, BESTFIT, FASTA, and TFASTA; see, e.g., Mount, D.W., Bioinformatics: Sequence and Genome Analysis, 2^{nd} Ed., Cold Spring Harbor Laboratory Press, Cold Spring Harbor, New York (2013))

Examples of algorithms that are suitable for determining percent sequence identity and sequence similarity are the BLAST and BLAST 2.0, FASTDB, or ALIGN algorithms, which are publically available (e.g., NCBI: National Center for Biotechnology Information). Those skilled in the art can determine appropriate parameters for aligning sequences. For example, the BLASTN program (for nucleotide sequences) can use as defaults a wordlength (W) of 11, an expectation (E) of 10, M=5, N=−4, and a comparison of both strands. Comparison of amino acid sequences using BLASTP can use as defaults a wordlength (W) of 3, an expectation (E) of 10, and the BLOSUM62 scoring matrix (see Henikoff and Henikoff, 1989, Proc Natl Acad Sci USA. 89:10915-9).

“Amino acid substitution” refers to the replacement of one amino acid in a polypeptide with another amino acid. A “conservative amino acid substitution” refers to the interchangeability of residues having similar side chains, and thus typically involves substitution of the amino acid in the polypeptide with amino acids within the same or similar defined class of amino acids. By way of example and not limitation, an amino acid with an aliphatic side chain may be substituted with another aliphatic amino acid, e.g., alanine, valine, leucine, isoleucine, and methionine; an amino acid with hydroxyl side chain is substituted with another amino acid with a hydroxyl side chain, e.g., serine and threonine; an amino acid having aromatic side chains is substituted with another amino acid having an aromatic side chain, e.g., phenylalanine, tyrosine, tryptophan, and histidine; an amino acid with a basic side chain is substituted with another amino acid with a basic side chain, e.g., lysine, arginine, and histidine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively.

“Amino acid insertion” refers to the incorporation of at least one amino acid into a predetermined amino acid sequence. An insertion can be the insertion of one or two amino acid residues; however, larger insertions of about three to about five, or up to about ten or more amino acid residues are contemplated herein.

“Amino acid deletion” refers to the removal of one or more amino acid residues from a predetermined amino acid sequence. A deletion can be the removal of one or two amino acid residues; however, larger deletions of about three to about five, or up to about ten or more amino acid residues are contemplated herein.

“Subject” refers to a mammal, including, but not limited to humans, non-human primates, and non-primates, such as goats, horses, and cows. In some embodiments, the terms “subject” and “patient” are used interchangeably herein in reference to a human subject.

“Therapeutically effective dose” or “therapeutically effective amount” or “effective dose”” refers to that quantity of a compound, including a biologic compound, or pharmaceutical composition that is sufficient to result in a desired activity upon administration to a mammal in need thereof. As used herein, with respect to the pharmaceutical compositions comprising an antibody, the term “therapeutically effective amount/dose” refers to the amount/dose of the antibody or pharmaceutical composition thereof that is sufficient to produce an effective response upon administration to a mammal.

“Pharmaceutically acceptable” refers to compounds or compositions which are generally safe, non-toxic and neither biologically nor otherwise undesirable, and includes a compound or composition that is acceptable for human pharmaceutical and veterinary use. The compound or composition may be approved or approvable by a regulatory agency or listed in the U.S. Pharmacopeia or other generally recognized pharmacopeia for use in animals, including humans.

“Pharmaceutically acceptable excipient, carrier or adjuvant” refers to an excipient, carrier or adjuvant that can be administered to a subject, together with at least one therapeutic agent (e.g., an antibody of the present disclosure), and which does not destroy the pharmacological activity thereof and is generally safe, nontoxic and neither biologically nor otherwise undesirable when administered in doses sufficient to deliver a therapeutic amount of the agent.

The term “treatment” is used interchangeably herein with the term “therapeutic method” and refers to both 1) therapeutic treatments or measures that cure, slow down, lessen symptoms of, and/or halt progression of a diagnosed pathologic conditions, disease or disorder, and 2) and prophylactic/preventative measures. Those in need of treatment may include individuals already having a particular medical disease or disorder as well as those who may ultimately acquire the disorder (i.e., those at risk or needing preventive measures).

The term “subject” or “patient” as used herein refers to any individual to which the subject methods are performed. Generally, the subject is human, although as will be appreciated by those in the art, the subject may be any animal.

In some embodiments, compounds of the present invention are able to cross the blood-brain barrier (BBB). The term “blood-brain barrier” or “BBB”, as used herein, refers to the BBB proper as well as to the blood-spinal barrier. The blood-brain barrier, which consists of the endothelium of the brain vessels, the basal membrane and neuroglial cells, acts to limit penetration of substances into the brain. In some embodiments, the brain/plasma ratio of total drug is at least approximately 0.01 after administration (e.g. oral or intravenous administration) to a patient. In some embodiments, the brain/plasma ratio of total drug is at least approximately 0.03. In some embodiments, the brain/plasma ratio of total drug is at least approximately 0.06. In some embodiments, the brain/plasma ratio of total drug is at least approximately 0.1. In some embodiments, the brain/plasma ratio of total drug is at least approximately 0.2.

The term “homologue,” especially “TREM homologue” as used herein refers to any member of a series of peptides or nucleic acid molecules having a common biological activity, including antigenicity/immunogenicity and inflammation regulatory activity, and/or structural domain and having sufficient amino acid or nucleotide sequence identity as defined herein. TREM homologues can be from either the same or different species of animals.

The term “variant” as used herein refers either to a naturally occurring allelic variation of a given peptide or a recombinantly prepared variation of a given peptide or protein in which one or more amino acid residues have been modified by amino acid substitution, addition, or deletion.

The term “derivative” as used herein refers to a variation of given peptide or protein that are otherwise modified, i.e., by covalent attachment of any type of molecule, preferably having bioactivity, to the peptide or protein, including non-naturally occurring amino acids.

### Description of Treatment Methods of the Present Invention

In one aspect, the present invention provides a method of treating a disease or disorder caused by and/or associated with an ABCD1 dysfunction in a human patient, the method comprising administering to the patient a compound that increases activity of TREM2. In some embodiments, the compound that increases activity of TREM2 is an agonist of TREM2. In some embodiments, the compound that increases activity of TREM2 is a compound that prevents the degradation of TREM2.

In one aspect, the present invention provides a method of treating a disease or disorder caused by and/or associated with an ABCD1 dysfunction in a human patient, the method comprising administering to the patient an effective amount of an agonist of TREM2. In some embodiments, administration of the agonist of TREM2 activates DAP12 signaling pathways in the patient, resulting in an increase in microglia proliferation, microglia survival and microglia phagocytosis, which in turn results in a slowing of disease progression. In some embodiments, the agonist of TREM2 is an antibody or a small molecule.

In some embodiments, the agonist of TREM2 activates TREM2/DAP12 signaling in myeloid cells, including monocytes, dendritic cells, microglial cells and macrophages. In some embodiments, an agonist of TREM2 activates, induces, promotes, stimulates, or otherwise increases one or more TREM2 activities. TREM2 activities that are activated or increased by the agonist, include but are not limited to: TREM2 binding to DAP12; DAP12 binding to TREM2; TREM2 phosphorylation, DAP12 phosphorylation; PI3K activation; increased levels of soluble TREM2 (sTREM2); increased levels of soluble CSF1R (sCSF1R); increased expression of one or more anti-inflammatory mediators (e.g., cytokines) selected from the group consisting of IL-12p70, IL-4, IL-6, and IL-1β; reduced expression of one or more pro-inflammatory mediators selected from the group consisting of IFN-α4, IFN-b, IL-6, IL-12 p70, IL-12 p40, IL-1β, TNF, TNF-α, IL-1β, IL-8, CRP, TGF-beta members of the chemokine protein families, IL-20 family members, IL-33, LIF, IFN-gamma, OSM, CNTF, TGF-beta, GM-CSF, IL-11, IL-12, IL-17, IL-18, and CRP; increased expression of one or more chemokines selected from the group consisting of CCL2, CCL4, CXCL10, CCL3 and CST7; reduced expression of TNF-α, IL-6, or both; extracellular signal-regulated kinase (ERK) phosphorylation; increased expression of C-C chemokine receptor 7 (CCR7); induction of microglial cell chemotaxis toward CCL19 and CCL21 expressing cells; an increase, normalization, or both of the ability of bone marrow-derived dendritic cells to induce antigen-specific T-cell proliferation; induction of osteoclast production, increased rate of osteoclastogenesis, or both; increasing the survival and/or function of one or more of dendritic cells, macrophages, microglial cells, M1 macrophages and/or microglial cells, activated M1 macrophages and/or microglial cells, M2 macrophages and/or microglial cells, monocytes, osteoclasts, Langerhans cells of skin, and Kupffer cells; induction of one or more types of clearance selected from the group consisting of apoptotic neuron clearance, nerve tissue debris clearance, non-nerve tissue debris clearance, bacteria or other foreign body clearance, disease-causing protein clearance, disease-causing peptide clearance, and disease-causing nucleic acid clearance; induction of phagocytosis of one or more of apoptotic neurons, nerve tissue debris, non-nerve tissue debris, bacteria, other foreign bodies, disease-causing proteins, disease-causing peptides, or disease-causing nucleic acids; normalization of disrupted TREM2/DAP12-dependent gene expression; recruitment of Syk, ZAP70, or both to the TREM2/DAP12 complex; Syk phosphorylation; increased expression of CD83 and/or CD86 on dendritic cells, macrophages, monocytes, and/or microglia; reduced secretion of one or more inflammatory cytokines selected from the group consisting of TNF-α, IL-1β, IL-6, MCP-1, IFN-α4, IFN-b, IL-1β, IL-8, CRP, TGF-beta members of the chemokine protein families, IL-20 family members, IL-33, LIF, IFN-gamma, OSM, CNTF, TGF-beta, GM-CSF, IL-11, IL-12, IL-17, IL-18, and CRP; reduced expression of one or more inflammatory receptors; increasing phagocytosis by macrophages, dendritic cells, monocytes, and/or microglia under conditions of reduced levels of MCSF; decreasing phagocytosis by macrophages, dendritic cells, monocytes, and/or microglia in the presence of normal levels of MCSF; increasing activity of one or more TREM2-dependent genes; increased levels of one or more of CSF1, CSF2 and IL-34; or any combination thereof. In another aspect, the invention provides a TREM2 agonist for the manufacture of a medicament for the treatment of a disease or disorder caused by and/or associated with an ABCD1 dysfunction.

In another aspect, the invention provides a TREM2 agonist for use in treating a disease or disorder caused by and/or associated with an ABCD1 dysfunction in a human patient.

### I. Diseases and Disorders

The methods of the present invention can be used to treat any disease or disorder related to a dysfunction in ABCD1. In some embodiments, the patient is selected for treatment based on a diagnosis that includes the presence of a mutation in an ABCD1 gene affecting the function of ABCD1. In some embodiments, the mutation in the ABCD1 gene is a mutation that causes a decrease in ABCD1 activity or a cessation of ABCD1 activity. In some embodiments, the disease or disorder is caused by a heterozygous ABCD1 mutation. In some embodiments, the disease or disorder is caused by a homozygous ABCD1 mutation. In some embodiments,
the disease or disorder is caused by a splice mutation in the ABCD1 gene. In some embodiments, the disease or disorder is caused by a missense mutation in the ABCD1 gene.

In some embodiments, the disease or disorder is a disease or disorder resulting from a change (e.g. increase, decrease or cessation) in the activity of ABCD1. In some embodiments, the disease or disorder is a disease or disorder resulting from a decrease or cessation in the activity of ABCD1. ABCD1 related activities that are changed in the disease or disorder include, but are not limited to peroxisomal import of fatty acids and/or fatty acyl-CoAs and production of adrenoleukodystrophy protein (ALDP).

In some embodiments, the disease or disorder is caused by a loss-of-function mutation in ABCD1. In some embodiments, the loss-of-function mutation results in a complete cessation of ABCD1 function. In some embodiments, the loss-of-function mutation results in a partial loss of ABCD1 function, or a decrease in ABCD1 activity. In some embodiments, the disease or disorder is caused by a homozygous mutation in ABCD1.

In some embodiments, the disease or disorder is a neurodegenerative disorder. In some embodiments, the disease or disorder is a neurodegenerative disorder caused by and/or associated with an ABCD1 dysfunction.

In some embodiments, the disease or disorder is an immunological disorder. In some embodiments, the disease or disorder is an immunological disorder caused by and/or associated with an ABCD1 dysfunction.

In some embodiments, the disease or disorder is selected from X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Alexander disease, fragile X-associated tremor ataxia syndrome (FXTAS), adult-onset autosomal dominant leukodystrophy (ADLD), and X-linked Charcot-Marie-Tooth disease (CMTX).

In some embodiments, the disease or disorder is selected from X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX), wherein any of the aforementioned diseases or disorders are present in a patient exhibiting ABCD1 dysfunction, or having a mutation in a gene affecting the function of ABCD1.

In some embodiments, the disease or disorder is X-linked adrenoleukodystrophy (x-ALD). In some embodiments, the x-ALD is a cerebral form of X-linked ALD (cALD).

In some embodiments, the disease or disorder is Addison disease wherein the patient has been found to have a mutation in one or more ABCD1 genes affecting ABCD1 function. In some embodiments, the disease or disorder is Addison disease, wherein the patient has a loss-of-function mutation in ABCD1.

In some embodiments, the disease or disorder is a white matter disease wherein the patient has been found to have a mutation in one or more ABCD1 genes affecting ABCD1 function. In some embodiments, the disease or disorder is a white matter disease, wherein the patient has a loss-of-function mutation in ABCD1.

In some embodiments, the disease or disorder is vanishing white matter disease wherein the patient has been found to have a mutation in one or more ABCD1 genes affecting ABCD1 function. In some embodiments, the disease or disorder is vanishing white matter disease, wherein the patient has a loss-of-function mutation in ABCD1.

In some embodiments, the disease or disorder is selected from Nasu-Hakola disease, Alzheimer's disease, frontotemporal dementia, multiple sclerosis, Guillain-Barre syndrome, amyotrophic lateral sclerosis (ALS), or Parkinson's disease, wherein any of the aforementioned diseases or disorders are present in a patient exhibiting ABCD1 dysfunction, or having a mutation in a gene affecting the function of ABCD1.

In some embodiments, the disease or disorder is Alzheimer's disease wherein the patient has been found to have a mutation in one or more ABCD1 genes affecting ABCD1 function. In some embodiments, the patient has been diagnosed with Alzheimer's disease based on neuropathology, and also has been found to have a mutation in one or more ABCD1 genes affecting ABCD1 function. In some embodiments, the disease or disorder is Alzheimer's disease, wherein the patient has a loss-of-function mutation in ABCD1.

In some embodiments, the disease or disorder is Nasu-Hakola disease wherein the patient has been found to have a mutation in one or more ABCD1 genes affecting ABCD1 function. In some embodiments, the patient has been diagnosed with Nasu-Hakola disease based on neuropathology, and also has been found to have a mutation in one or more ABCD1 genes affecting ABCD1 function. In some embodiments, the disease or disorder is Nasu-Hakola disease, wherein the patient has a loss-of-function mutation in ABCD1.

In some embodiments, the disease or disorder is Parkinson's disease wherein the patient has been found to have a mutation in one or more ABCD1 genes affecting ABCD1 function. In some embodiments, the patient has been diagnosed with Parkinson's disease based on neuropathology, and also has been found to have a mutation in one or more ABCD1 genes affecting ABCD1 function. In some embodiments, the disease or disorder is Parkinson's disease, wherein the patient has a loss-of-function mutation in ABCD1.

In some embodiments, the disease or disorder is multiple sclerosis wherein the patient has been found to have a mutation in one or more ABCD1 genes affecting ABCD1 function. In some embodiments, the patient has been diagnosed with multiple sclerosis based on neuropathology, and also has been found to have a mutation in one or more ABCD1 genes affecting ABCD1 function. In some embodiments, the disease or disorder is multiple sclerosis, wherein the patient has a loss-of-function mutation in ABCD1.

In some embodiments, the disease or disorder is ALS wherein the patient has been found to have a mutation in one or more ABCD1 genes affecting ABCD1 function. In some embodiments, the patient has been diagnosed with ALS based on neuropathology, and also has been found to have a mutation in one or more ABCD1 genes affecting ABCD1 function. In some embodiments, the disease or disorder is ALS, wherein the patient has a loss-of-function mutation in ABCD1.

In some embodiments, the disease or disorder is Guillain-Barre syndrome wherein the patient has been found to have a mutation in one or more ABCD1 genes affecting ABCD1 function. In some embodiments, the patient has been diagnosed with Guillain-Barre syndrome based on neuropathology, and also has been found to have a mutation in one or more ABCD1 genes affecting ABCD1 function. In some embodiments, the disease or disorder is Guillain-Barre syndrome, wherein the patient has a loss-of-function mutation in ABCD1.

In some embodiments, the patient also possesses a mutation in one or more of NOTCH3, HTRA1, TREX1, ARSA, EIF2B1, EIF2B2, EIF2B3, EIF2B4, and EIF2B5.

In some embodiments, the disease or disorder presents one or more symptoms selected from abnormal motor control, parkinsonism, slow movement (bradykinesia), involuntary trembling (tremor), muscle stiffness (rigidity), cognitive decline, dementia, inability to speak, inability to walk, memory loss, personality changes, seizures, depression, loss of executive function, loss of impulse control, loss of attention span, adrenal insufficiency, vision impairment, hearing impairment, sexual dysfunction, impaired adrenocortical function, attention-deficit, hyperactivity, and incontinence.

In one aspect, the present invention provides a method of treating x-ALD in a human patient, the method comprising administering to the patient a compound that increases activity of TREM2. In some embodiments, the compound that increases activity of TREM2 is an agonist of TREM2. In some embodiments, the compound that increases activity of TREM2 is a compound that prevents the degradation of TREM2.

In one aspect, the present invention provides a method of treating x-ALD in a human patient, the method comprising administering to the patient an effective amount of an agonist of TREM2. In some embodiments, administration of the agonist of TREM2 activates DAP12 signaling pathways in the patient, resulting in an increase in microglia proliferation, microglia survival and microglia phagocytosis, which in turn results in a slowing of disease progression in x-ALD. In some embodiments, the agonist of TREM2 is an antibody or a small molecule.

In another aspect, the invention provides a TREM2 agonist for the manufacture of a medicament for the treatment of a disease or disorder related to an ABCD1 dysfunction. In another aspect, the invention provides a TREM2 agonist for the manufacture of a medicament for the treatment of x-ALD.

In another aspect, the invention provides a TREM2 agonist for use in treating a disease or disorder related to an ABCD1 dysfunction in a human patient. In another aspect, the invention provides a TREM2 agonist for use in treating x-ALD in a human patient.

### Huntington's Disease

In one aspect, the present invention provides a method of treating Huntington's disease in a human patient, the method comprising administering to the patient an effective amount of an agonist of TREM2. In some embodiments, administration of the agonist of TREM2 activates DAP12 signaling pathways in the patient, resulting in an increase in microglia proliferation, microglia survival and microglia phagocytosis, which in turn results in a slowing of disease progression in Huntington's disease. In some embodiments, the agonist of TREM2 is an antibody or a small molecule. In some embodiments, the agonist of TREM2 is an antibody or a small molecule disclosed elsewhere herein. In some embodiments, the agonist of TREM2 is an antibody disclosed elsewhere herein. In some embodiments, the agonist of TREM2 is a small molecule disclosed elsewhere herein. In another aspect, the invention provides a TREM2 agonist for the manufacture of a medicament for the treatment of Huntington's disease. In another aspect, the invention provides a TREM2 agonist for use in treating Huntington's disease in a human patient.

### II. Antibodies

In one aspect, the present invention provides a method of treating ALSP in a human patient, the method comprising administering to the patient an effective amount of an antigen binding protein or an antibody, or an antigen-binding fragment thereof, which increases the activity of TREM2. In some embodiments, the antibody is an agonist of TREM2. In some embodiments, the antibody is an agonist of TREM2 that specifically binds to and activates human TREM2.

The TREM2 agonist antibodies specifically bind to human TREM2 (SEQ ID NO: 1) or an extra cellular domain (ECD) of human TREM2 (e.g. ECD set forth in SEQ ID NO: 2), for example with an equilibrium dissociation constant (K_{D}) less than 50 nM, less than 25 nM, less than 10 nM, or less than 5 nM. In some embodiments, the TREM2 agonist antibodies do not cross-react with other TREM proteins, such as human TREM1. In some embodiments, the TREM2 agonist antibodies do not bind to human TREM1 (SEQ ID NO: 4).

In some embodiments, the TREM2 antibody specifically bind to human TREM2 human TREM2 residues 19-174. In some embodiments, the TREM2 antibody specifically bind to IgV region of human TREM2, for example human TREM2 residues 19-140.

In certain embodiments, anti-TREM2 antibodies of the present disclosure bind to one or more amino acids within amino acid residues 29-112 of human TREM 2 (SEQ ID NO: 1), or within amino acid residues on a TREM2 protein corresponding to amino acid residues 29-112 of SEQ ID NO: 1. In some embodiments, anti-TREM2 antibodies of the present disclosure bind to one or more amino acids within amino acid residues 29-41 of human TREM 2 (SEQ ID NO: 1), or within amino acid residues on a TREM2 protein corresponding to amino acid residues 29-41 of SEQ ID NO: 1. In some embodiments, anti-TREM2 antibodies of the present disclosure bind to one or more amino acids within amino acid residues 47-69 of human TREM 2 (SEQ ID NO: 1), or within amino acid residues on a TREM2 protein corresponding to amino acid residues 47-69 of SEQ ID NO: 1. In some embodiments, anti-TREM2 antibodies of the present disclosure bind to one or more amino acids within amino acid residues 76-86 of human TREM 2 (SEQ ID NO: 1), or within amino acid residues on a TREM2 protein corresponding to amino acid residues 76-86 of SEQ ID NO: 1. In some embodiments, anti-TREM2 antibodies of the present disclosure bind to one or more amino acids within amino acid residues 91-100 of human TREM2 (SEQ ID NO: 1), or within amino acid residues on a TREM2 protein corresponding to amino acid residues 91-100 of SEQ ID NO: 1. In some embodiments, anti-TREM2 antibodies of the present disclosure bind to one or more amino acids within amino acid residues 99-115 of human TREM 2 (SEQ ID NO: 1), or within amino acid residues on a TREM2 protein corresponding to amino acid residues 99-115 of SEQ ID NO: 1. In some embodiments, anti-TREM2 antibodies of the present disclosure bind to one or more amino acids within amino acid residues 104-112 of human TREM 2 (SEQ ID NO: 1), or within amino acid residues on a TREM2 protein corresponding to amino acid residues 104-112 of SEQ ID NO: 1. In some embodiments, anti-TREM2 antibodies of the present disclosure bind to one or more amino acids within amino acid residues 114-118 of human TREM 2 (SEQ ID NO: 1), or within amino acid residues on a TREM2 protein corresponding to amino acid residues 114-118 of SEQ ID NO: 1. In some embodiments, anti-TREM2 antibodies of the present disclosure bind to one or more amino acids within amino acid residues 130-171 of human TREM 2 (SEQ ID NO: 1), or within amino acid residues on a TREM2 protein corresponding to amino acid residues 130-171 of SEQ ID NO: 1. In some embodiments, anti-TREM2 antibodies of the present disclosure bind to one or more amino acids within amino acid residues 139-153 of human TREM 2 (SEQ ID NO: 1), or within amino acid residues on a TREM2 protein corresponding to amino acid residues 139-153 of SEQ ID NO: 1. In some embodiments, anti-TREM2 antibodies of the present disclosure bind to one or more amino acids within amino acid residues 139-146 of human TREM 2 (SEQ ID NO: 1), or within amino acid residues on a TREM2 protein corresponding to amino acid residues 139-146 of SEQ ID NO: 1. In some embodiments, anti-TREM2 antibodies of the present disclosure bind to one or more amino acids within amino acid residues 130-144 of human TREM 2 (SEQ ID NO: 1), or within amino acid residues on a TREM2 protein corresponding to amino acid residues 130-144 of SEQ ID NO: 1. In some embodiments, anti-TREM2 antibodies of the present disclosure bind to one or more amino acids within amino acid residues 158-171 of human TREM 2 (SEQ ID NO: 1), or within amino acid residues on a TREM2 protein corresponding to amino acid residues 158-171 of SEQ ID NO: 1.

In some embodiments, anti-TREM2 antibodies of the present disclosure bind to one or more amino acids within amino acid residues 43-50 of human TREM 2 (SEQ ID NO: 1), or within amino acid residues on a TREM2 protein corresponding to amino acid residues 43-50 of SEQ ID NO: 1. In some embodiments, anti-TREM2 antibodies of the present disclosure bind to one or more amino acids within amino acid residues 49-57 of human TREM 2 (SEQ ID NO: 1), or within amino acid residues on a TREM2 protein corresponding to amino acid residues 49-57 of SEQ ID NO: 1. In some embodiments, anti-TREM2 antibodies of the present disclosure bind to one or more amino acids within amino acid residues 139-146 of human TREM 2 (SEQ ID NO: 1), or within amino acid residues on a TREM2 protein corresponding to amino acid residues 139-146 of SEQ ID NO: 1. In some embodiments, anti-TREM2 antibodies of the present disclosure bind to one or more amino acids within amino acid residues 140-153 of human TREM 2 (SEQ ID NO: 1), or within amino acid residues on a TREM2 protein corresponding to amino acid residues 140-153 of SEQ ID NO: 1. In some embodiments, the TREM2 antibody specifically binds to the stalk region of human TREM2, for example amino acid residues 145-174 of human TREM2.

In some embodiments, the antibody, or an antigen-binding fragment thereof, specifically binds TREM2 and prevents the degradation or cleavage of TREM2.

In some embodiments, the antibody is a polyclonal antibody. In some embodiments, the antibody is a monoclonal antibody. In some embodiments, the antibody is a chimeric antibody. In some embodiments, the antibody is a humanized antibody. In some embodiments, the antibody is a human antibody, particularly a fully human antibody. In some embodiments, the antibody is a bispecific or other multivalent antibody. In some embodiments, the antibody is a single chain antibody.

In some embodiments, a TREM2 activating antibody comprise a light chain variable region comprising complementarity determining regions CDRL1, CDRL2, and CDRL3 and a heavy chain variable region comprising complementarity determining regions CDRH1, CDRH2, and CDRH3 described herein.

In certain embodiments, the TREM2 agonist antigen binding proteins of the invention comprise at least one light chain variable region comprising a CDRL1, CDRL2, and CDRL3, and at least one heavy chain variable region comprising a CDRH1, CDRH2, and CDRH3 from an anti-TREM2 agonist antibody described herein.

In some embodiments, a TREM2 activating antibody comprise a light chain variable region and a heavy chain variable region described herein. The light chain and heavy chain variable regions or CDRs may be from any of the anti-TREM2 antibodies or a variant thereof described herein.

### Sequence Information

A. PCT Patent Application Publication No. WO2018/195506A1

In some embodiments, the TREM2 agonist is an antigen binding protein or an antibody, or an antigen-binding fragment thereof, as described in PCT Patent Application Publication No. WO2018/195506A1, which is incorporated by reference herein, in its entirety.

In some embodiments, the TREM2 agonist antigen binding protein comprises a CDRL1 or a variant thereof having one, two, three or four amino acid substitutions; a CDRL2, or a variant thereof having one, two, three or four amino acid substitutions; a CDRL3, or a variant thereof having one, two, three or four amino acid substitutions; a CDRH1, or a variant thereof having one, two, three or four amino acid substitutions; a CDRH2, or a variant thereof having one, two, three or four amino acid substitutions; and a CDRH3, or a variant thereof having one, two, three or four amino acid substitutions, where the amino acid sequences of the CDRL1, CDRL2, CDRL3, CDRH1, CDRH2, and CDRH3 are provided in TABLES A1 and A2 below, along with exemplary light chain and variable regions.

TABLE A1
Exemplary Anti-Human TREM2 Antibody Light Chain Variable Region Amino Acid Sequences

| Ab ID. | VL Group | VL Amino Acid Sequence | CDRL1 | CDRL2 | CDRL3 |
|---|---|---|---|---|---|
| 12G10 | LV-01 | QAVPTQPSSLSASPGVLASLTCTLRSGINVG | TLRSGI | YKSDSD | MIWYSS |
| | | TYRIYWYQQKPGSPPQYLLRYKSDSDKQQ | NVGTY | KQQGS | AVV |
| | | GSGVPSRFSGSKDASANAGILLISGLQSEDE | RIY | (SEQ ID | (SEQ ID |
| | | ADYYCMIWYSSAVVFGGGTKLTVL | (SEQ ID | NO: 19) | NO: 31) |
| | | (SEQ ID NO: 46) | NO: 5) | | |
| 26A10 | LV-02 | SYELTQPPSVSVSPGQTASITCSGDKLGDKY | SGDKL | QDSKRP | QAWDS |
| | | VCWYQQKPGQSPVLVIYQDSKRPSGIPERF | GDKYVC | S | NTVV |
| | | SGSNSGNTATLTISGTQAMDEADYYCQAW | (SEQ ID | (SEQ ID | (SEQ ID |
| | | DSNTVVFGGGTKLTVL (SEQ ID NO: 47) | NO: 6) | NO: 20) | NO: 32) |
| 26C10 | LV-03 | SFELTQPPSVSVSPGQTASITCSGDKLGDKY | SGDKL | QDTKRP | QAWDSS |
| | | VCWYQQKPGQSPMLVIYQDTKRPSGIPERF | GDKYVC | S | TVV |
| | | SGSNSGNTATLTISGTQAMDEADYYCQAW | (SEQ ID | (SEQ ID | (SEQ ID |
| | | DSSTVVFGGGTKLTVL (SEQ ID NO: 48) | NO: 6) | NO: 21) | NO: 33) |
| 26F2 | LV-04 | SYELTQPPSVSVSPGQTASITCSGDKLGDKY | SGDKL | QDSKRP | QAWDSS |
| | | VCWYQQKPGQSPVLVIFQDSKRPSGIPERFS | GDKYVC | S | TVV |
| | | GSNSGNTATLTISGTQAMDEADYYCQAWD | (SEQ ID | (SEQ ID | (SEQ ID |
| | | SSTVVFGGGTKLTVL (SEQ ID NO: 49) | NO: 6) | NO: 20) | NO: 33) |
| 33B12 | LV-05 | SYELTQPPSVSVSPGQTASITCSGDKLGDKY | SGDKL | QDSKRP | QAWDSS |
| | | VCWYQQKPGQSPVLVIYQDSKRPSGIPERF | GDKYVC | S | TVV |
| | | SGSNSGNTATLTISGTQAMDEADYYCQAW | (SEQ ID | (SEQ ID | (SEQ ID |
| | | DSSTVVFGGGTKLTVL (SEQ ID NO: 50) | NO: 6) | NO: 20) | NO: 33) |
| 24C12 | LV-06 | GIVMTQSPDSLAVSLGERATINCKSSRSVLY | KSSRSV | WASTRE | QQYYIT |
| | | SSNNKNYLAWYQQKPGQPPKVLIYWASTR | LYSSN | S | PIT |
| | | ESGVPDRFSGSGSGTDFTLTISSLQAEDVAV | KNYLA | (SEQ ID | (SEQ ID |
| | | YNCQQYYITPITFGQGTRLEIK | (SEQ ID | NO: 22) | NO: 34) |
| | | (SEQ ID NO: 51) | NO: 7) | | |
| 24G6 | LV-07 | DIVMTQSPDSLAVSLGERATINCKSSQSVLY | KSSQSV | WASTRE | QQYYST |
| | | SSNNKHFLAWYQQKPGQPPKLLIYWASTR | LYSSN | S | PLT |
| | | ESGVPDRFSGSGSGTDFTLTISSLQAEDVAF | KFIFLA | (SEQ ID | (SEQ ID |
| | | YYCQQYYSTPLTFGGGTKVEIK | (SEQ ID | NO: 22) | NO: 35) |
| | | (SEQ ID NO: 52) | NO: 8) | | |
| 24A10 | LV-08 | DIVMTQSPDSLAVSLGERATITCKSSHNVL | KSSHN | WASTRE | HQYYST |
| | | YSSNNKNYLAWYQQKPGQPPKLLIYWAST | VLYSSN | S | PCS |
| | | RESGVPDRFSGSGSGTDFTLTISSLQAEDVA | NKNYLA | (SEQ ID | (SEQ ID |
| | | VYYCHQYYSTPCSFGQGTKLEIK | (SEQ ID | NO: 22) | NO: 36) |
| | | (SEQ ID NO: 53) | NO: 9) | | |
| 10E3 | LV-09 | EIVMTQSPATLSVSPGERATLSCRASQSVSS | RASQS | GASTRA | LQDNN |
| | | NLAWFQQKPGQAPRLLIYGASTRATGIPAR | VSSNLA | T | WPPT |
| | | FSVSGSGTEFTLTISSLQSEDFAFYYCLQDN | (SEQ ID | (SEQ ID | (SEQ ID |
| | | NWPPTFGPGTKVDIK (SEQ ID NO: 54) | NO: 10) | NO: 23) | NO: 37) |
| 13E7 | LV-10 | EIVMTQSPATLSVSPGERATLSCRASQSVSS | RASQS | GASTRA | LQDNN |
| 14C12 | | NLAWFQQKPGQAPRLLIYGASTRATGIPAR | VSSNLA | T | WPPT |
| | | FSVSGSGTEFTLTISSLQSEDFAVYYCLQDN | (SEQ ID | (SEQ ID | (SEQ ID |
| | | NWPPTFGPGTKVDIK (SEQ ID NO: 55) | NO: 10) | NO: 23) | NO: 37) |
| 25F12 | LV-11 | EKVMTQSPATLSVSPGERATLSCRASQSVN | RASQS | GASTRA | QQYNN |
| | | NNLAWYQQKPGQAPRLLIYGASTRATGIPA | VNNNL | T | WPRT |
| | | RFSGSGSGTEFTLTISSLQSEDFAVYYCQQY | A | (SEQ ID | (SEQ ID |
| | | NNWPRTFGQGTKVEIK (SEQ ID NO: 56) | (SEQ ID | NO: 23) | NO: 38) |
| | | | NO: 11) | | |
| 32E3 | LV-12 | EFVLTQSPGTLSLSPGERATLSCRASQIISSN | RASQIIS | SASSRA | QQFDSS |
| | | YLAWYQQKPGQAPRLLIYSASSRATGIPDR | SNYLA | T | PIT |
| | | FSGSGSGTDFTLTISRLEPEDFAVYYCQQFD | (SEQ ID | (SEQ ID | (SEQ ID |
| | | SSPITFGRGTRLDIK (SEQ ID NO: 57) | NO: 12) | NO: 24) | NO: 39) |
| 24F4 | LV-13 | EIVLTQSPGTLSLSPGERATLSCRASQSVSSS | RASQS | GASSRA | QQYDTS |
| | | YLAWYQQKPGQAPRLLIYGASSRATGIPDR | VSSSYLA | T | PFT |
| | | FSGSGSGTDFTLTISRLEPEDFALYYCQQYD | (SEQ ID | (SEQ ID | (SEQ ID |
| | | TSPFTFGPGTKVDIK (SEQ ID NO: 58) | NO: 13) | NO: 25) | NO: 40) |
| 16B8 | LV 14 | DIQMTQSPSSVSASVGDRVTVTCRASQDIN | RASQDI | AASSLQ | QQSNSF |
| | | SWLAWYQQKPGKAPKLLIYAASSLQTGVP | NSWLA | T | PIT |
| | | SRFSGSGSGTDFTLTISSLQPEDFATYSCQQS | (SEQ ID | (SEQ ID | (SEQ ID |
| | | NSFPITFGQGTRLEIK (SEQ ID NO: 59) | NO: 14) | NO: 26) | NO: 41) |
| 4C5 | LV-15 | DIQMTQSPSSVSASVGDRVTITCRASQGISN | RASQGI | AASSLQ | QQADSF |
| | | WLAWYQQKPGKAPKLLIYAASSLQVGVPL | SNWLA | V | PRN |
| | | RFSGSGSGTDFTLTISSLQPEDFATYYCQQA | (SEQ ID | (SEQ ID | (SEQ ID |
| | | DSFPRNFGQGTKLEIK (SEQ ID NO: 60) | NO: 15) | NO: 27) | NO: 42) |
| 6E7 | LV-16 | DIQMTQSPSSVSASVGDRVTITCRASQGISS | RASQGI | AASSLQ | QQADSF |
| | | WLAWYQQKPGKAPKLLIYAASSLQNGVPS | SSWLA | N | PRT |
| | | RFSGSGSGTDFTLTISSLQPEDFATYFCQQA | (SEQ ID | (SEQ ID | (SEQ ID |
| | | DSFPRTFGQGTKLEIK (SEQ ID NO: 61) | NO: 16) | NO: 28) | NO: 43) |
| 5E3 | LV-17 | DIQMTQSPSSLSASVGDRVTITCRASQGISN | RASQGI | AASSLQ | QQYSTY |
| | | YLAWFQQKPGKAPKSLIYAASSLQSGVPSK | SNYLA | S | PFT |
| | | FSGSGSGTDFTLTISSLQPEDFATYYCQQYS | (SEQ ID | (SEQ ID | (SEQ ID |
| | | TYPFTFGPGTKVDIK (SEQ ID NO: 62) | NO: 17) | NO: 29) | NO: 44) |
| 4G10 | LV-18 | DIQMTQSPSSLSASVGDRVTITCRASQGIRN | RASQGI | AASSLPS | LQHNSY |
| | | DLGWYQQKPGNAPKRLIYAASSLPSGVPSR | RNDLG | (SEQ ID | PWT |
| | | FSGSGSGPEFTLTISSLQPEDFATYYCLQHN | (SEQ ID | NO: 30) | (SEQ ID |
| | | SYPWTFGQGTKVEIT (SEQ ID NO: 63) | NO: 18) | | NO: 45) |

TABLE A2
Exemplary Anti-Human TREM2 Antibody Heavy Chain Variable Region Amino Acid Sequences

| Ab ID. | VH Group | VH Amino Acid Sequence | CDRH1 | CDRH2 | CDRH3 |
|---|---|---|---|---|---|
| 12G10 | HV-01 | EVQLLESGGGLVQPGGSLRLSCAASGFTFS | SYAMS | AIGGGG | FYIAVA |
| 24C12 | | SYAMSWVRQAPGKGLEWVSAIGGGGVST | (SEQ ID | VSTYCA | GSHFDY |
| | | YCADSVKGRFTISRDNSKNTLYLQMNSLRA | NO: 77) | DSVKG | (SEQ ID |
| | | EDTAVYYCAKFYIAVAGSHFDYWGQGTLV | | (SEQ ID | NO: 95) |
| | | TVSS (SEQ ID NO: 110) | | NO: 87) | |
| 26A10 | HV-02 | EVQLVESGGALVQRGGSLRLSCAASRFTFS | SFGMS | YISSSSF | EGGLTM |
| | | SFGMSWVRQAPGKGLEWVSYISSSSFTIYY | (SEQ ID | TIYYAD | VRGVSS |
| | | ADSVKGRFTISRDNAKNSFYLQMNSLRDED | NO: 78) | SVKG | YGLDV |
| | | TAVYYCAREGGLTMVRGVSSYGLDVWGQ | | (SEQ ID | (SEQ ID |
| | | GTTVTVSS (SEQ ID NO: 111) | | NO: 88) | NO: 96) |
| 26C10 | HV-03 | EVQLVESGGALVQPGGSLRLSCAASGFTFS | SFGMS | YISSSSF | EGGITM |
| | | SFGMSWVRQAPGKGLEWVSYISSSSFTIYY | (SEQ ID | TIYYAD | VRGVSS |
| | | ADSVKGRFTISRDNAKNSFYLQMNSLRDED | NO: 78) | SVKG | YGMDV |
| | | TAVYFCVREGGITMVRGVSSYGMDVWGQ | | (SEQ ID | (SEQ ID |
| | | GTTVTVSS (SEQ ID NO: 112) | | NO: 88) | NO: 97) |
| 26F2 | HV-04 | EVQLVESGGALVQPGGSLRLSCAASGFTFS | SFGMS | YISSSSF | EGGITM |
| | | SFGMSWVRQAPGKGLEWISYISSSSFTIYYA | (SEQ ID | TIYYAD | VRGVSS |
| | | DSVKGRFTISRDNAKNSFYLQMNSLRDEDT | NO: 78) | SVKG | YGMDV |
| | | AVYFCAREGGITMVRGVSSYGMDVWGQG | | (SEQ ID | (SEQ ID |
| | | TTVTVSS (SEQ ID NO: 113) | | NO: 88) | NO: 97) |
| 33B12 | HV-05 | EVQLVESGGALVQPGGSLRLSCAASGFTFS | SFGMS | YISKSSF | EGGLTM |
| | | SFGMSWVRQAPGKGLEWVSYISKSSFTIYY | (SEQ ID | TIYYAD | VRGVSS |
| | | ADSVKGRFTISRDNAKNSFYLQMNSLRDED | NO: 78) | SVKG | YGLDV |
| | | TAVYYCAREGGLTMVRGVSSYGLDVWGQ | | (SEQ ID | (SEQ ID |
| | | GTTVTVSS (SEQ ID NO: 114) | | NO: 89) | NO: 96) |
| 24G6 | HV-06 | EVQLLESGGGLVQPGGSLRLSCAASGFTFS | SYAMS | AISGSGG | AYTPMA |
| | | SYAMSWVRQAPGKGLEWVSAISGSGGSTY | (SEQ ID | STYYAD | FFDY |
| | | YADSVKGRFTISRDNSKNTLYLQMNSLRAE | NO: 77) | SVKG | (SEQ ID |
| | | DTAVYYCAKAYTPMAFFDYWGQGTLVTV | | (SEQ ID | NO: 98) |
| | | SS (SEQ ID NO: 115) | | NO: 90) | |
| 24A10 | HV-07 | EVQVLESGGGLVQPGGSLRLSCAASGFTFS | NYAMS | AISGSGG | GGWELF |
| | | NYAMSWVRQAPGKGLEWVSAISGSGGSTY | (SEQ ID | STYYAD | Y |
| | | YADSVKGRFTISRDNSKNTLYLQMNSLRAE | NO: 79) | SVKG | (SEQ ID |
| | | DTAVYYCAKGGWELFYWGQGTLVTVSS | | (SEQ ID | NO: 99) |
| | | (SEQ ID NO: 116) | | NO: 90) | |
| 10E3 | HV-08 | EVQLVQSGAEVKKPGESLMISCKGSGYSFT | NYWIG | ITYPGDS | RRQGIW |
| | | NYWIGWVRQMPGKGLEWMGITYPGDSDTR | (SEQ ID | DTRYSP | GDALDI |
| | | YSPSFQGQVTISADKSISTAYLQWSSLKASD | NO: 80) | SFQG | (SEQ ID |
| | | TAMYFCARRRQGIWGDALDIWGQGTLVTV | | (SEQ ID | NO: 100) |
| | | SS (SEQ ID NO: 117) | | NO: 91) | |
| 13E7 | HV-09 | EVQLVQSGAEVKKPGESLMISCKGSGYSFT | SWIG | ITYPGDS | RRQGIW |
| 14C12 | | SYWIGWVRQMPGKGLEWMGITYPGDSDTR | (SEQ ID | DTRYSP | GDALDF |
| | | YSPSFQGQVTISADKSISTAYLQWSSLKASD | NO: 81) | SFQG | (SEQ ID |
| | | TAMYFCARRRQGIWGDALDFWGQGTLVT | | (SEQ ID | NO: 101) |
| | | VSS (SEQ ID NO: 118) | | NO: 91) | |
| 25F12 | HV-10 | QVQLQQWGAGLLKPSETLSLTCAVYGGSF | SYYWS | EINHSG | EGYYDI |
| | | SSYYWSWIRQPPGKGLEWIGEINHSGNTNY | (SEQ ID | NTNYNP | LTGYHD |
| | | NPSLKSRVTISVDTSKNQFSLKLSSVTAADT | NO: 82) | SLKS | AFDI |
| | | AVYYCAREGYYDILTGYHDAFDIWDQGTM | | (SEQ ID | (SEQ ID |
| | | VTVFS (SEQ ID NO: 119) | | NO: 92) | NO: 102) |
| 32E3 | HV-11 | EVQLVQSGAEVKKPGESLKISCKGSGYSFT | SWIG | IIYPGDS | HDIIPAA |
| | | SYWIGWVRQMPGKGLEWMGITYPGDSDTR | (SEQ ID | DTRYSP | PGAFDI |
| | | YSPSFQGQVTISADKSISTAYLQWSTLKASD | NO: 81) | SFQG | (SEQ ID |
| | | TAIYYCARHDIIPAAPGAFDIWGQGTMVTV | | (SEQ ID | NO: 103) |
| | | SS (SEQ ID NO: 120) | | NO: 91) | |
| 24F4 | HV-12 | EVQLVQSGAEVKKPGESLKISCKGSGYTFT | SWIG | IIYPGDS | QAIAVT |
| | | SYWIGWVRQMPGKGLEWMGITYPGDSDTR | (SEQ ID | DTRYSP | GLGGFD |
| | | YSPSFQGQVTISVDKSSSTAYLQWSSLKAS | NO: 81) | SFQG | P |
| | | DTAIYYCTRQAIAVTGLGGFDPWGQGTLV | | (SEQ ID | (SEQ ID |
| | | TVSS (SEQ ID NO: 121) | | NO: 91) | NO: 104) |
| 16B8 | HV-13 | QVQLVQSGAEVKKPGASVKVSCKASGYTF | NYGIS | WISAYN | RGYSYG |
| | | TNYGISWVRQAPGQGLEWMGWISAYNGN | (SEQ ID | GNTNYA | SFDY |
| | | TNYAQKLQGRVTMTTDTSTSTVYMELRSL | NO: 83) | QKLQG | (SEQ ID |
| | | RSDDTAVYYCARRGYSYGSFDYWGQGTL | | (SEQ ID | NO: 105) |
| | | VTVSS (SEQ ID NO: 122) | | NO: 93) | |
| 4C5 | HV-14 | EVQLVQSGAEVKKPGESLKISCKGSGHSFT | NYWIA | IIYPGDS | QRTFYY |
| | | NYWIAWVRQMPGKGLEWMGITYPGDSDTR | (SEQ ID | DTRYSP | DSSGYF |
| | | YSPSFQGQVTISADKSISTAYLQWSSLKASD | NO: 84) | SFQG | DY |
| | | TAVYFCARQRTFYYDSSGYFDYWGQGTLV | | (SEQ ID | (SEQ ID |
| | | TVSS (SEQ ID NO: 123) | | NO: 91) | NO: 106) |
| 6E7 | HV-15 | EVQLVQSGAEVKKPGESLKISCKGSGYSFT | SYWIA | ITYPGDS | QRTFYY |
| | | SYWIAWVRQMPGKGLEWMGITYPGDSDTR | (SEQ ID | DTRYSP | DSSDYF |
| | | YSPSFQGQVTISADKSISTAYLQWSSLKASD | NO: 85) | SFQG | DY |
| | | TAMYFCARQRTFYYDSSDYFDYWGQGTL | | (SEQ ID | (SEQ ID |
| | | VTVSS (SEQ ID NO: 124) | | NO: 91) | NO: 107) |
| 5E3 | HV-16 | QVQLVQSGAEVKKPGASVKVSCKASGYTF | GYYIH | WINPYS | DGGYLA |
| | | TGYYIHWVRQAPGLGLEWMGWINPYSGG | (SEQ ID | GGTTSA | LYGTDV |
| | | TTSAQKFQGRVTMTRDTSISSAYMELSRLR | NO: 86) | QKFQG | (SEQ ID |
| | | SDDTAVYYCARDGGYLALYGTDVWGQGT | | (SEQ ID | NO: 108) |
| | | TVTVSS (SEQ ID NO: 125) | | NO: 94) | |
| 4G10 | HV-17 | EVQLVQSGAEVKKPGESLKISCKGSGYSFP | SYWIA | ITYPGDS | QGIEVT |
| | | SYWIAWVRQMPGKGLEWMGITYPGDSDTR | (SEQ ID | DTRYSP | GTGGLD |
| | | YSPSFQGQVTISADKSISTAFLKWSSLKASD | NO: 85) | SFQG | V |
| | | TAMYFCARQGIEVTGTGGLDVWGQGTTVT | | (SEQ ID | (SEQ ID |
| | | VSS (SEQ ID NO: 126) | | NO: 91) | NO: 109) |

As noted above, a TREM2 agonist antigen binding protein may comprise one or more of the CDRs presented in TABLE A1 (light chain CDRs; i.e. CDRLs) and TABLE A2 (heavy chain CDRs, i.e. CDRHs).

In some embodiments, the TREM2 agonist antigen binding protein comprises one or more light chain CDRs selected from (i) a CDRL1 selected from SEQ ID NOS:5 to 18, (ii) a CDRL2 selected from SEQ ID NOS:19 to 30, and (iii) a CDRL3 selected from SEQ ID NOS:31 to 45, and (iv) a CDRL of (i), (ii) and (iii) that contains one or more, e.g., one, two, three, four or more amino acid substitutions (e.g., conservative amino acid substitutions), deletions or insertions of no more than five, four, three, two, or one amino acids. In these and other embodiments, the TREM2 agonist antigen binding proteins comprise one or more heavy chain CDRs selected from (i) a CDRH1 selected from SEQ ID NOS:77 to 86, (ii) a CDRH2 selected from SEQ ID NOS:87 to 94, and (iii) a CDRH3 selected from SEQ ID NOS:95 to 109, and (iv) a CDRH of (i), (ii) and (iii) that contains one or more, e.g., one, two, three, four or more amino acid substitutions (e.g., conservative amino acid substitutions), deletions or insertions of no more than five, four, three, two, or one amino acids amino acids.

In some embodiments, the TREM2 agonist antigen binding protein may comprise 1, 2, 3, 4, 5, or 6 variant forms of the CDRs listed in TABLES A1 and A2, each having at least 80%, 85%, 90% or 95% sequence identity to a CDR sequence listed in TABLES A1 and A2. In some embodiments, the TREM2 agonist antigen binding protein includes 1, 2, 3, 4, 5, or 6 of the CDRs listed in TABLES A1 and A2, each differing by no more than 1, 2, 3, 4 or 5 amino acids from the CDRs listed in these tables.

In some embodiments, the TREM2 agonist antigen binding protein comprises a CDRL1 comprising a sequence selected from SEQ ID NOS:5-18 or a variant thereof having one, two, three or four amino acid substitutions; a CDRL2 comprising a sequence selected from SEQ ID NOS: 19-30 or a variant thereof having one, two, three or four amino acid substitutions; a CDRL3 comprising a sequence selected from SEQ ID NOS:31-45 or a variant thereof having one, two, three or four amino acid substitutions; a CDRH1 comprising a sequence selected from SEQ ID NOS:77-86 or a variant thereof having one, two, three or four amino acid substitutions; a CDRH2 comprising a sequence selected from SEQ ID NOS:87-94 or a variant thereof having one, two, three or four amino acid substitutions; and a CDRH3 comprising a sequence selected from SEQ ID NOS:95-109 or a variant thereof having one, two, three or four amino acid substitutions.

In some embodiments, the TREM2 agonist antigen binding proteins of the invention comprise a CDRL1 comprising a sequence selected from SEQ ID NOS:5-18; a CDRL2 comprising a sequence selected from SEQ ID NOS:19-30; a CDRL3 comprising a sequence selected from SEQ ID NOS:31-45; a CDRH1 comprising a sequence selected from SEQ ID NOS:77-86; a CDRH2 comprising a sequence selected from SEQ ID NOS:87-94; and a CDRH3 comprising a sequence selected from SEQ ID NOS:95-109.

In some embodiments, the TREM2 agonist antigen binding protein comprise a light chain variable region comprising a CDRL1, a CDRL2, and a CDRL3, wherein:
(a) CDRL1, CDRL2, and CDRL3 have the sequence of SEQ ID NOS: 5, 19, and 31, respectively;
(b) CDRL1, CDRL2, and CDRL3 have the sequence of SEQ ID NOS:6, 20, and 32, respectively;
(c) CDRL1, CDRL2, and CDRL3 have the sequence of SEQ ID NOS:6, 21, and 33, respectively;
(d) CDRL1, CDRL2, and CDRL3 have the sequence of SEQ ID NOS:6, 20, and 33, respectively;
(e) CDRL1, CDRL2, and CDRL3 have the sequence of SEQ ID NOS:7, 22, and 34, respectively;
(f) CDRL1, CDRL2, and CDRL3 have the sequence of SEQ ID NOS:8, 22, and 35, respectively;
(g) CDRL1, CDRL2, and CDRL3 have the sequence of SEQ ID NOS:9, 22, and 36, respectively;
(h) CDRL1, CDRL2, and CDRL3 have the sequence of SEQ ID NOS: 10, 23, and 37, respectively;
(i) CDRL1, CDRL2, and CDRL3 have the sequence of SEQ ID NOS: 11, 23, and 38, respectively;
(j) CDRL1, CDRL2, and CDRL3 have the sequence of SEQ ID NOS: 12, 24, and 39, respectively;
(k) CDRL1, CDRL2, and CDRL3 have the sequence of SEQ ID NOS: 13, 25, and 40, respectively;
(l) CDRL1, CDRL2, and CDRL3 have the sequence of SEQ ID NOS: 14, 26, and 41, respectively;
(m) CDRL1, CDRL2, and CDRL3 have the sequence of SEQ ID NOS: 15, 27, and 42, respectively;
(n) CDRL1, CDRL2, and CDRL3 have the sequence of SEQ ID NOS: 16, 28, and 43, respectively;
(o) CDRL1, CDRL2, and CDRL3 have the sequence of SEQ ID NOS: 17, 29, and 44, respectively, or
(p) CDRL1, CDRL2, and CDRL3 have the sequence of SEQ ID NOS: 18, 30, and 45, respectively.

In some embodiments, the TREM2 agonist antigen binding protein comprises a heavy chain variable region comprising a CDRH1, a CDRH2, and a CDRH3, wherein:
(a) CDRH1, CDRH2, and CDRH3 have the sequence of SEQ ID NOS:77, 87, and 95, respectively;
(b) CDRH1, CDRH2, and CDRH3 have the sequence of SEQ ID NOS:78, 88, and 96, respectively;
(c) CDRH1, CDRH2, and CDRH3 have the sequence of SEQ ID NOS:78, 88, and 97, respectively;
(d) CDRH1, CDRH2, and CDRH3 have the sequence of SEQ ID NOS:78, 89, and 96, respectively;
(e) CDRH1, CDRH2, and CDRH3 have the sequence of SEQ ID NOS:77, 90, and 98, respectively;
(f) CDRH1, CDRH2, and CDRH3 have the sequence of SEQ ID NOS:79, 90, and 99, respectively;
(g) CDRH1, CDRH2, and CDRH3 have the sequence of SEQ ID NOS:80, 91, and 100, respectively;
(h) CDRH1, CDRH2, and CDRH3 have the sequence of SEQ ID NOS:81, 91, and 101, respectively;
(i) CDRH1, CDRH2, and CDRH3 have the sequence of SEQ ID NOS:82, 92, and 102, respectively;
(j) CDRH1, CDRH2, and CDRH3 have the sequence of SEQ ID NOS:81, 91, and 103, respectively;
(k) CDRH1, CDRH2, and CDRH3 have the sequence of SEQ ID NOS:81, 91, and 104, respectively;
(l) CDRH1, CDRH2, and CDRH3 have the sequence of SEQ ID NOS:83, 93, and 105, respectively;
(m) CDRH1, CDRH2, and CDRH3 have the sequence of SEQ ID NOS:84, 91, and 106, respectively;
(n) CDRH1, CDRH2, and CDRH3 have the sequence of SEQ ID NOS:85, 91, and 107, respectively;
(o) CDRH1, CDRH2, and CDRH3 have the sequence of SEQ ID NOS:86, 94, and 108, respectively; or
(p) CDRH1, CDRH2, and CDRH3 have the sequence of SEQ ID NOS:85, 91, and 109, respectively.

In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein:
(a) CDRL1, CDRL2, and CDRL3 have the sequence of SEQ ID NOS:5, 19, and 31, respectively, and CDRH1, CDRH2, and CDRH3 have the sequence of SEQ ID NOS:77, 87, and 95, respectively;
(b) CDRL1, CDRL2, and CDRL3 have the sequence of SEQ ID NOS:6, 20, and 32, respectively, and CDRH1, CDRH2, and CDRH3 have the sequence of SEQ ID NOS:78, 88, and 96, respectively;
(c) CDRL1, CDRL2, and CDRL3 have the sequence of SEQ ID NOS:6, 21, and 33, respectively, and CDRH1, CDRH2, and CDRH3 have the sequence of SEQ ID NOS:78, 88, and 97, respectively;
(d) CDRL1, CDRL2, and CDRL3 have the sequence of SEQ ID NOS:6, 20, and 33, respectively, and CDRH1, CDRH2, and CDRH3 have the sequence of SEQ ID NOS:78, 88, and 97, respectively;
(e) CDRL1, CDRL2, and CDRL3 have the sequence of SEQ ID NOS:6, 20, and 33, respectively, and CDRH1, CDRH2, and CDRH3 have the sequence of SEQ ID NOS:78, 89, and 96, respectively;
(f) CDRL1, CDRL2, and CDRL3 have the sequence of SEQ ID NOS:7, 22, and 34, respectively, and CDRH1, CDRH2, and CDRH3 have the sequence of SEQ ID NOS:77, 87, and 95, respectively;
(g) CDRL1, CDRL2, and CDRL3 have the sequence of SEQ ID NOS:8, 22, and 35, respectively, and CDRH1, CDRH2, and CDRH3 have the sequence of SEQ ID NOS:77, 90, and 98, respectively;
(h) CDRL1, CDRL2, and CDRL3 have the sequence of SEQ ID NOS:9, 22, and 36, respectively, and CDRH1, CDRH2, and CDRH3 have the sequence of SEQ ID NOS:79, 90, and 99, respectively;
(i) CDRL1, CDRL2, and CDRL3 have the sequence of SEQ ID NOS: 10, 23, and 37, respectively, and CDRH1, CDRH2, and CDRH3 have the sequence of SEQ ID NOS:80, 91, and 100, respectively;
(j) CDRL1, CDRL2, and CDRL3 have the sequence of SEQ ID NOS: 10, 23, and 37, respectively, and CDRH1, CDRH2, and CDRH3 have the sequence of SEQ ID NOS:81, 91, and 101, respectively;
(k) CDRL1, CDRL2, and CDRL3 have the sequence of SEQ ID NOS:11, 23, and 38, respectively, and CDRH1, CDRH2, and CDRH3 have the sequence of SEQ ID NOS:82, 92, and 102, respectively;
(l) CDRL1, CDRL2, and CDRL3 have the sequence of SEQ ID NOS: 12, 24, and 39, respectively, and CDRH1, CDRH2, and CDRH3 have the sequence of SEQ ID NOS:81, 91, and 103, respectively;
(m) CDRL1, CDRL2, and CDRL3 have the sequence of SEQ ID NOS: 13, 25, and 40, respectively, and CDRH1, CDRH2, and CDRH3 have the sequence of SEQ ID NOS:81, 91, and 104, respectively;
(n) CDRL1, CDRL2, and CDRL3 have the sequence of SEQ ID NOS: 14, 26, and 41, respectively, and CDRH1, CDRH2, and CDRH3 have the sequence of SEQ ID NOS:83, 93, and 105, respectively;
(o) CDRL1, CDRL2, and CDRL3 have the sequence of SEQ ID NOS: 15, 27, and 42, respectively, and CDRH1, CDRH2, and CDRH3 have the sequence of SEQ ID NOS:84, 91, and 106, respectively;
(p) CDRL1, CDRL2, and CDRL3 have the sequence of SEQ ID NOS: 16, 28, and 43, respectively, and CDRH1, CDRH2, and CDRH3 have the sequence of SEQ ID NOS:85, 91, and 107, respectively;
(q) CDRL1, CDRL2, and CDRL3 have the sequence of SEQ ID NOS: 17, 29, and 44, respectively, and CDRH1, CDRH2, and CDRH3 have the sequence of SEQ ID NOS:86, 94, and 108, respectively; or
(r) CDRL1, CDRL2, and CDRL3 have the sequence of SEQ ID NOS:18, 30, and 45, respectively, and CDRH1, CDRH2, and CDRH3 have the sequence of SEQ ID NOS:85, 91, and 109, respectively.

In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOS:10, 23, and 37, respectively, and CDRH1, CDRH2, and CDRH3 have the sequence of SEQ ID NOS:80, 91, and 100, respectively. In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOS:10, 23, and 37, respectively, and CDRH1, CDRH2, and CDRH3 have the sequence of SEQ ID NOS:81, 91, and 101, respectively. In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOS: 15, 27, and 42, respectively, and CDRH1, CDRH2, and CDRH3 have the sequence of SEQ ID NOS:84, 91, and 106, respectively. In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOS:16, 28, and 43, respectively, and CDRH1, CDRH2, and CDRH3 have the sequence of SEQ ID NOS:85, 91, and 107, respectively. In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOS:17, 29, and 44, respectively, and CDRH1, CDRH2, and CDRH3 have the sequence of SEQ ID NOS:86, 94, and 108, respectively. In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOS:8, 22, and 35, respectively, and CDRH1, CDRH2, and CDRH3 have the sequence of SEQ ID NOS:77, 90, and 98, respectively.

In some embodiments, the TREM2 agonist antigen binding proteins comprise a light chain variable region comprising a sequence selected from SEQ ID NOS:46-63 and a heavy chain variable region comprising a sequence selected from SEQ ID NOS: 110-126.In some embodiments, the TREM2 agonist antigen binding protein comprises a light chain variable region comprising the sequence of SEQ ID NO:46 and a heavy chain variable region comprising the sequence of SEQ ID NO:110. In some embodiments, the TREM2 agonist antigen binding protein comprises a light chain variable region comprising the sequence of SEQ ID NO:47 and a heavy chain variable region comprising the sequence of SEQ ID NO:111. In some embodiments, the TREM2 agonist antigen binding protein comprises a light chain variable region comprising the sequence of SEQ ID NO:48 and a heavy chain variable region comprising the sequence of SEQ ID NO: 112. In some embodiments, the TREM2 agonist antigen binding protein comprises a light chain variable region comprising the sequence of SEQ ID NO:49 and a heavy chain variable region comprising the sequence of SEQ ID NO: 113. In some embodiments, the TREM2 agonist antigen binding protein comprises a light chain variable region comprising the sequence of SEQ ID NO:50 and a heavy chain variable region comprising the sequence of SEQ ID NO:114. In some embodiments, the TREM2 agonist antigen binding protein comprises a light chain variable region comprising the sequence of SEQ ID NO:51 and a heavy chain variable region comprising the sequence of SEQ ID NO: 110. In some embodiments, the TREM2 agonist antigen binding protein comprises a light chain variable region comprising the sequence of SEQ ID NO:53 and a heavy chain variable region comprising the sequence of SEQ ID NO: 116. In some embodiments, the TREM2 agonist antigen binding protein comprises a light chain variable region comprising the sequence of SEQ ID NO:54 and a heavy chain variable region comprising the sequence of SEQ ID NO: 117. In some embodiments, the TREM2 agonist antigen binding protein comprises a light chain variable region comprising the sequence of SEQ ID NO:55 and a heavy chain variable region comprising the sequence of SEQ ID NO:118. In some embodiments, the TREM2 agonist antigen binding protein comprises a light chain variable region comprising the sequence of SEQ ID NO:56 and a heavy chain variable region comprising the sequence of SEQ ID NO: 119. In some embodiments, the TREM2 agonist antigen binding protein comprises a light chain variable region comprising the sequence of SEQ ID NO:57 and a heavy chain variable region comprising the sequence of SEQ ID NO: 120. In some embodiments, the TREM2 agonist antigen binding protein comprises a light chain variable region comprising the sequence of SEQ ID NO:58 and a heavy chain variable region comprising the sequence of SEQ ID NO:121. In some embodiments, the TREM2 agonist antigen binding protein comprises a light chain variable region comprising the sequence of SEQ ID NO:59 and a heavy chain variable region comprising the sequence of SEQ ID NO:122. In some embodiments, the TREM2 agonist antigen binding protein comprises a light chain variable region comprising the sequence of SEQ ID NO:60 and a heavy chain variable region comprising the sequence of SEQ ID NO: 123. In some embodiments, the TREM2 agonist antigen binding protein comprises a light chain variable region comprising the sequence of SEQ ID NO:61 and a heavy chain variable region comprising the sequence of SEQ ID NO: 124. In some embodiments, the TREM2 agonist antigen binding protein comprises a light chain variable region comprising the sequence of SEQ ID NO:62 and a heavy chain variable region comprising the sequence of SEQ ID NO: 125. In some embodiments, the TREM2 agonist antigen binding protein comprises a light chain variable region comprising the sequence of SEQ ID NO:63 and a heavy chain variable region comprising the sequence of SEQ ID NO:126. In yet another embodiment, the TREM2 agonist antigen binding protein comprises a light chain variable region comprising the sequence of SEQ ID NO:52 and a heavy chain variable region comprising the sequence of SEQ ID NO:115.

In some embodiments, the TREM2 agonist antigen binding protein may comprise a light chain variable region selected from LV-01, LV-02, LV-03, LV-04, LV-05, LV-06, LV-07, LV-08, LV-09, LV-10, LV-11, LV-12, LV-13, LV-14, LV-15, LV-16, LV-17, and LV-18, as shown in TABLE A1, and/or a heavy chain variable region selected from HV-01, HV-02, HV-03, HV-04, HV-05, HV-06, HV-07, HV-08, HV-09, HV-10, HV-11, HV-12, HV-13, HV-14, HV-15, HV-16, and HV-17, as shown in TABLE A2, and functional fragments, derivatives, muteins and variants of these light chain and heavy chain variable regions.

In some embodiments, each of the light chain variable regions listed in TABLE Almay be combined with any of the heavy chain variable regions listed in TABLE A2 to form an anti-TREM2 binding domain of the antigen binding proteins of the invention. Examples of such combinations include, but are not limited to: LV-01 (SEQ ID NO:46) and HV-01 (SEQ ID NO: 110); LV-02 (SEQ ID NO:47) and HV-02 (SEQ ID NO: 111); LV-03 (SEQ ID NO:48) and HV-03 (SEQ ID NO: 112); LV-04 (SEQ ID NO:49) and HV-04 (SEQ ID NO: 113); LV-05 (SEQ ID NO:50) and HV-05 (SEQ ID NO: 114); LV-06 (SEQ ID NO:51) and HV-0 (SEQ ID NO:110); LV-07 (SEQ ID NO:52) and HV-06 (SEQ ID NO: 115); LV-08 (SEQ ID NO:53) and HV-07 (SEQ ID NO: 116); LV-09 (SEQ ID NO:54) and HV-08 (SEQ ID NO: 117); LV-10 (SEQ ID NO:55) and HV-09 (SEQ ID NO: 118); LV-11 (SEQ ID NO:56) and HV-10 (SEQ ID NO: 119); LV-12 (SEQ ID NO:57) and HV-11 (SEQ ID NO: 120); LV-13 (SEQ ID NO:58) and HV-12 (SEQ ID NO:121); LV-14 (SEQ ID NO:59) and HV-13 (SEQ ID NO: 122); LV-15 (SEQ ID NO:60) and HV-14 (SEQ ID NO: 123); LV-16 (SEQ ID NO:61) and HV-15 (SEQ ID NO: 124); LV-17 (SEQ ID NO:62) and HV-16 (SEQ ID NO: 125); and LV-18 (SEQ ID NO:63) and HV-17 (SEQ ID NO: 126).

In certain embodiments, the TREM2 agonist antigen binding proteins of the invention comprise a light chain variable region comprising the sequence of LV-09 (SEQ ID NO:54) and a heavy chain variable region comprising the sequence of HV-08 (SEQ ID NO: 117). In some embodiments, the TREM2 agonist antigen binding proteins of the invention comprise a light chain variable region comprising the sequence of LV-10 (SEQ ID NO:55) and a heavy chain variable region comprising the sequence of HV-09 (SEQ ID NO: 118). In other embodiments, the TREM2 agonist antigen binding proteins of the invention comprise a light chain variable region comprising the sequence of LV-15 (SEQ ID NO:60) and a heavy chain variable region comprising the sequence of HV-14 (SEQ ID NO: 123). In still other embodiments, the TREM2 agonist antigen binding proteins of the invention comprise a light chain variable region comprising the sequence of LV-16 (SEQ ID NO:61) and a heavy chain variable region comprising the sequence of HV-15 (SEQ ID NO: 124). In some embodiments, the TREM2 agonist antigen binding proteins of the invention comprise a light chain variable region comprising the sequence of LV-17 (SEQ ID NO:62) and a heavy chain variable region comprising the sequence of HV-16 (SEQ ID NO: 125). In certain embodiments, the TREM2 agonist antigen binding proteins of the invention comprise a light chain variable region comprising the sequence of LV-07 (SEQ ID NO:52) and a heavy chain variable region comprising the sequence of HV-06 (SEQ ID NO: 115).

In some embodiments, the TREM2 agonist antigen binding proteins comprise a light chain variable region comprising a sequence of contiguous amino acids that differs from the sequence of a light chain variable region in TABLE A1, i.e. a VL selected from LV-01, LV-02, LV-03, LV-04, LV-05, LV-06, LV-07, LV-08, LV-09, LV-10, LV-11, LV-12, LV-13, LV-14, LV-15, LV-16, LV-17, or LV-18, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The light chain variable region in some TREM2 agonist antigen binding proteins comprises a sequence of amino acids that has at least 70%, at least 75%, at least 80%, at least 85%, at least 90%, at least 95%, at least 97% or at least 99% sequence identity to the amino acid sequences of SEQ ID NOS:46-63 (i.e. the light chain variable regions in TABLE A1). In one embodiment, the TREM2 agonist antigen binding protein comprises a light chain variable region comprising a sequence that is at least 90% identical to a sequence selected from SEQ ID NOS:46-63. In another embodiment, the TREM2 agonist antigen binding protein comprises a light chain variable region comprising a sequence that is at least 95% identical to a sequence selected from SEQ ID NOS:46-63. In yet another embodiment, the TREM2 agonist antigen binding protein comprises a light chain variable region comprising a sequence selected from SEQ ID NOS:46-63. In some embodiments, the TREM2 agonist antigen binding protein comprises a light chain variable region comprising a sequence of SEQ ID NO:54. In other embodiments, the TREM2 agonist antigen binding protein comprises a light chain variable region comprising a sequence of SEQ ID NO:55. In yet other embodiments, the TREM2 agonist antigen binding protein comprises a light chain variable region comprising a sequence of SEQ ID NO:60. In still other embodiments, the TREM2 agonist antigen binding protein comprises a light chain variable region comprising a sequence of SEQ ID NO:61. In certain embodiments, the TREM2 agonist antigen binding protein comprises a light chain variable region comprising a sequence of SEQ ID NO:62. In other embodiments, the TREM2 agonist antigen binding protein comprises a light chain variable region comprising a sequence of SEQ ID NO:52.

In these and other embodiments, the TREM2 agonist antigen binding proteins comprise a heavy chain variable region comprising a sequence of contiguous amino acids that differs from the sequence of a heavy chain variable region in TABLE A2, i.e., a VH selected from HV-01, HV-02, HV-03, HV-04, HV-05, HV-06, HV-07, HV-08, HV-09, HV-10, HV-11, HV-12, HV-13, HV-14, HV-15, HV-16, or HV-17, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The heavy chain variable region in some TREM2 agonist antigen binding proteins comprises a sequence of amino acids that has at least 70%, at least 75%, at least 80%, at least 85%, at least 90%, at least 95%, at least 97% or at least 99% sequence identity to the amino acid sequences of SEQ ID NOS:110-126 (i.e. the heavy chain variable regions in TABLE A2). In one embodiment, the TREM2 agonist antigen binding protein comprises a heavy chain variable region comprising a sequence that is at least 90% identical to a sequence selected from SEQ ID NOS: 110-126. In another embodiment, the TREM2 agonist antigen binding protein comprises a heavy chain variable region comprising a sequence that is at least 95% identical to a sequence selected from SEQ ID NOS: 110-126. In yet another embodiment, the TREM2 agonist antigen binding protein comprises a heavy chain variable region comprising a sequence selected from SEQ ID NOS: 110-126. In some embodiments, the TREM2 agonist antigen binding protein comprises a heavy chain variable region comprising a sequence of SEQ ID NO: 117. In other embodiments, the TREM2 agonist antigen binding protein comprises a heavy chain variable region comprising a sequence of SEQ ID NO: 118. In yet other embodiments, the TREM2 agonist antigen binding protein comprises a heavy chain variable region comprising a sequence of SEQ ID NO:123. In still other embodiments, the TREM2 agonist antigen binding protein comprises a heavy chain variable region comprising a sequence of SEQ ID NO: 124. In certain embodiments, the TREM2 agonist antigen binding protein comprises a heavy chain variable region comprising a sequence of SEQ ID NO:125. In other embodiments, the TREM2 agonist antigen binding protein comprises a heavy chain variable region comprising a sequence of SEQ ID NO: 115.

In some embodiments, variants of the anti-TREM2 antibodies can be generated by substituting one or more amino acids in the light chain or heavy chain variable regions to address chemical liabilities (e.g., aspartate isomerization, asparagine deamidation, tryptophan and methionine oxidation) or correct covariance violations (see e.g., WO 2012/125495, which is hereby incorporated by reference in its entirety). Such variants can have improved biophysical, expression, and/or stability properties as compared with the parental antibody. In some embodiments, the TREM2 agonist antigen binding proteins of the invention comprise a light chain variable region and/or heavy chain variable region having one or more of the amino acid substitutions set forth in any of TABLES A3-A4 below.

In some embodiments, additional variants of the anti-TREM2 antibodies described herein can be generated by affinity modulating any of the anti-TREM2 antibodies described herein. An “affinity-modulated antibody” is an antibody that comprises one or more amino acid substitutions in its light chain variable region sequence and/or heavy chain variable region sequence that increases or decreases the affinity of the antibody for the target antigen as compared to the parental antibody that does not contain the amino acid substitutions. Antibody affinity modulation methods are known to those of skill in the art and can include CDR walking mutagenesis (Yang et al., J. Mol. Biol., 254, 392-403, 1995), chain shuffling (Marks et al., Bio/Technology, 10, 779-783, 1992), use of mutation strains of E. coli (Low et al., J. Mol. Biol., 250, 350-368, 1996), DNA shuffling (Patten et al., Curr. Opin. Biotechnol., 1997, 8:724-733), phage display (Thompson et al., J. Mol. Biol., 1996, 256:7-88), PCR techniques (Crameri, et al., Nature, 1998, 391:288-291), and other mutagenesis strategies (Barbas et al., Proc Nat. Acad. Sci. USA 91:3809-3813, 1994; Schier et al., Gene 169:147-155, 1995; Yelton et al., J. Immunol. 155:1994-2004, 1995; Jackson et al., J. Immunol. 154(7):3310-9, 1995; and Hawkins et al., J. Mol. Biol., 1992, 226:889-896). Methods of affinity modulation are discussed in Hoogenboom, Trends in Biotechnology, 1995, 15:62-70, and Vaughan et al., Nature Biotechnology, 1998, 16535-539,. One specific method for generating affinity-modulated variants of the anti-TREM2 antibodies described herein is the use of a yeast-display Fab mutagenesis library.

In some embodiments, the TREM2 agonist antigen binding proteins comprise a light chain variable region that is a variant of a light chain variable region of any of the anti-TREM2 antibodies described herein. Thus, in some embodiments, the light chain variable region of the TREM2 agonist antigen binding proteins comprises a sequence that is at least 90% identical, at least 91% identical, at least 92% identical, at least 93% identical, at least 94% identical, or at least 95% identical to a sequence selected from SEQ ID NOS:46-63. In some embodiments, the TREM2 agonist antigen binding proteins can comprise a light chain variable region from any of the engineered anti-TREM2 antibody variants set forth in TABLES A3-A4 below.

In some embodiments, the TREM2 agonist antigen binding protein comprises a light chain variable region comprising the sequence of SEQ ID NO:54 with a mutation at one or more amino acid positions 64, 79, 80, 85, 94, and/or 100. In some such embodiments, the mutation is V64G, V64A, Q79E, Q79D, S80P, S80A, F85V, F85L, F85A, F85D, F85I, F85L, F85M, F85T, W94F, W94Y, W94S, W94T, W94A, W94H, W94I, W94Q, P100R, P100Q, P100G, or combinations thereof. In another embodiment, the TREM2 agonist antigen binding protein comprises a light chain variable region comprising the sequence of SEQ ID NO:55 with a mutation at one or more amino acid positions 64, 79, 80, 94, and/or 100. Such mutations can include V64G, V64A, Q79E, Q79D, S80P, S80A, W94F, W94Y, W94S, W94T, W94A, W94H, W94I, W94Q, P100R, P100Q, P100G, or combinations thereof. In certain embodiments, the mutation is V64G, V64A, Q79E, S80P, S80A, W94Y, W94S, P100R, P100Q, or combinations thereof. In another embodiment, the TREM2 agonist antigen binding protein comprises a light chain variable region comprising the sequence of SEQ ID NO:60 with a mutation at one or more amino acid positions 60, 92, and/or 93. The mutation in such embodiments can be selected from L60S, L60P, L60D, L60A, D92E, D92Q, D92T, D92N, S93A, S93N, S93Q, S93V, or combinations thereof. In yet another embodiment, the TREM2 agonist antigen binding protein comprises a light chain variable region comprising the sequence of SEQ ID NO:61 with a mutation at one or more amino acid positions 56, 57, 92, and/or 93. In such embodiments, the mutation can be N56S, N56T, N56Q, N56E, G57A, G57V, D92E, D92Q, D92T, D92N, S93A, S93N, S93Q, S93V, or combinations thereof. In certain embodiments, the mutation is N56S, N56Q, G57A, D92E, D92Q, S93A, or combinations thereof. In still another embodiment, the TREM2 agonist antigen binding protein comprises a light chain variable region comprising the sequence of SEQ ID NO:62 with a mutation at amino acid position 36, 46, 61 and/or 100. Such mutations can include F36Y, S46L, S46R, S46V, S46F, K61R, P100Q, P100G, P100R or combinations thereof. In particular embodiments, the mutation is F36Y, K61R, P100Q, or combinations thereof. In another embodiment, the TREM2 agonist antigen binding protein comprises a light chain variable region comprising the sequence of SEQ ID NO:52 with a mutation at amino acid position 91, which can be selected from F91V, F91I, F91T, F91L, or F91D. In one embodiment, the mutation is F91V.

In some embodiments, the TREM2 agonist antigen binding proteins comprise a heavy chain variable region that is a variant of a heavy chain variable region from any of the anti-TREM2 antibodies described herein. Thus, in some embodiments, the heavy chain variable region of the TREM2 agonist antigen binding proteins comprises a sequence that is at least 90% identical, at least 91% identical, at least 92% identical, at least 93% identical, at least 94% identical, or at least 95% identical to a sequence selected from SEQ ID NOS:110-126. For instance, the TREM2 agonist antigen binding proteins can comprise a heavy chain variable region from any of the engineered anti-TREM2 antibody variants set forth in TABLES A3-A4 below. In one embodiment, the TREM2 agonist antigen binding protein comprises a heavy chain variable region comprising the sequence of SEQ ID NO: 117 with a mutation at one or more amino acid positions 19, 55, 56, 57, 58, and/or 104. In some such embodiments, the mutation is M19K, M19R, M19T, M19E, M19N, M19Q, D55E, D55Q, D55N, D55T, S56A, S56Q, S56V, D57S, D57E, D57Q, T58A, T58V, W104F, W104Y, W104T, W104S, W104A, W104H, W1041, W104Q, or combinations thereof. In another embodiment, the TREM2 agonist antigen binding protein comprises a heavy chain variable region comprising the sequence of SEQ ID NO:118 with a mutation at one or more amino acid positions 19, 55, 56, 57, 58, and/or 104. Such mutations can include M19K, M19R, M19T, M19E, M19N, M19Q, D55E, D55Q, D55N, D55T, S56A, S56Q, S56V, D57S, D57E, D57Q, T58A, T58V, W104F, W104Y, W104T, W104S, W104A, W104H, W1041, W104Q, or combinations thereof. In certain embodiments, the mutation is M19K, D55E, S56A, D57E, T58A, W104Y, W104T, or combinations thereof. In another embodiment, the TREM2 agonist antigen binding protein comprises a heavy chain variable region comprising the sequence of SEQ ID NO:123 with a mutation at one or more amino acid positions 27, 55, 56, 57, 58, 105, and/or 106. In some embodiments, the mutation is selected from H27Y, H27D, H27F, H27N, D55E, D55Q, D55N, D55T, S56A, S56Q, S56V, D57S, D57E, D57Q, T58A, T58V, D105E, D105Q, D105T, D105N, D105G, S106A, S106Q, S106V, S106T, or combinations thereof. In yet another embodiment, the TREM2 agonist antigen binding protein comprises a heavy chain variable region comprising the sequence of SEQ ID NO:124 with a mutation at one or more amino acid positions 55, 56, 57, 58, 105, and/or 106. The mutation in such embodiments can be selected from D55E, D55Q, D55N, D55T, S56A, S56Q, S56V, D57S, D57E, D57Q, T58A, T58V, D105E, D105Q, D105T, D105N, D105G, S106A, S106Q, S106V, S106T, or combinations thereof. In certain embodiments, the mutation is D55E, D55Q, S56A, D57E, T58A, D105E, D105N, S106A, or combinations thereof. In still another embodiment, the TREM2 agonist antigen binding protein comprises a heavy chain variable region comprising the sequence of SEQ ID NO: 125 with a mutation at one or more amino acid positions 43, 76, 85, 99, 100, and/or 116. Such mutations can include L43Q, L43K, L43H, I76T, R85S, R85G, R85N, R85D, D99E, D99Q, D99S, D99T, G100A, G100Y, G100V, T116L, T116M, T116P, T116R, or combinations thereof. In certain embodiments, the mutation is L43Q, R85S, D99E, G100A, G100Y, Ti 16L, or combinations thereof. In another embodiment, the TREM2 agonist antigen binding protein comprises a heavy chain variable region comprising the sequence of SEQ ID NO: 115 with a mutation at amino acid position 62 and/or 63. In such embodiments, the mutation can be selected from D62E, D62Q, D62T, D62N, S63A, S63Q, S63V, or combinations thereof. In some embodiments, the mutation is D62E, D62Q, S63A, or combinations thereof. In some embodiments, the TREM2 agonist antigen binding proteins comprise a light chain variable region and/or heavy chain variable region from any of the anti-TREM2 variant antibodies set forth in TABLES A3, A4, A4, A5, A6, A7, and A8. Accordingly, in some embodiments, the light chain variable region of the TREM2 agonist antigen binding proteins comprises a sequence that is at least 90% identical, at least 91% identical, at least 92% identical, at least 93% identical, at least 94% identical, or at least 95% identical to a sequence selected from SEQ ID NOS:61, 153-162, and 295-300. In these and other embodiments, the heavy chain variable region of the TREM2 agonist antigen binding proteins comprises a sequence that is at least 90% identical, at least 91% identical, at least 92% identical, at least 93% identical, at least 94% identical, or at least 95% identical to a sequence selected from SEQ ID NOS: 124, 180-190, and 307-312.

In some embodiments, the TREM2 agonist antigen binding protein comprises a light chain variable region comprising the sequence of SEQ ID NO:54 with a mutation at one or more amino acid positions 64, 79, 80, 85, 94, and/or 100. Such mutations can include V64G, V64A, Q79E, Q79D, S80P, S80A, F85V, F85L, F85A, F85D, F85I, F85L, F85M, F85T, W94F, W94Y, W94S, W94T, W94A, W94H, W94I, W94Q, P100R, P100Q, P100G, or combinations thereof. In these and other embodiments, the TREM2 agonist antigen binding protein comprises a heavy chain variable region comprising the sequence of SEQ ID NO: 117 with a mutation at one or more amino acid positions 19, 55, 56, 57, 58, and/or 104. In certain embodiments, the mutation is selected from M19K, M19R, M19T, M19E, M19N, M19Q, D55E, D55Q, D55N, D55T, S56A, S56Q, S56V, D57S, D57E, D57Q, T58A, T58V, W104F, W104Y, W104T, W104S, W104A, W104H, W1041, W104Q, or combinations thereof.

In other embodiments, the TREM2 agonist antigen binding protein comprises a light chain variable region comprising the sequence of SEQ ID NO:55 with a mutation at one or more amino acid positions 64, 79, 80, 94, and/or 100. In some embodiments, the mutation is selected from V64G, V64A, Q79E, Q79D, S80P, S80A, W94F, W94Y, W94S, W94T, W94A, W94H, W94I, W94Q, P100R, P100Q, P100G, or combinations thereof. In certain embodiments, the mutation is selected from V64G, V64A, Q79E, S80P, S80A, W94Y, W94S, P100R, P100Q, or combinations thereof. For instance, in some embodiments, the TREM2 agonist antigen binding protein comprises a light chain variable region comprising the sequence of SEQ ID NO:55 with one or more mutations selected from V64G, Q79E, S80P, W94Y, and P100Q. In these and other embodiments, the TREM2 agonist antigen binding protein comprises a heavy chain variable region comprising the sequence of SEQ ID NO: 118 with a mutation at one or more amino acid positions 19, 55, 56, 57, 58, and/or 104. Such mutations can include M19K, M19R, M19T, M19E, M19N, M19Q, D55E, D55Q, D55N, D55T, S56A, S56Q, S56V, D57S, D57E, D57Q, T58A, T58V, W104F, W104Y, W104T, W104S, W104A, W104H, W1041, W104Q, or combinations thereof. In certain embodiments, the mutation is selected from M19K, D55E, S56A, D57E, T58A, W104Y, W104T, or combinations thereof.

In certain other embodiments, the TREM2 agonist antigen binding protein comprises a light chain variable region comprising the sequence of SEQ ID NO:60 with a mutation at one or more amino acid positions 60, 92, and/or 93. The mutation can be selected from L60S, L60P, L60D, L60A, D92E, D92Q, D92T, D92N, S93A, S93N, S93Q, S93V, or combinations thereof. In these and other embodiments, the TREM2 agonist antigen binding protein comprises a heavy chain variable region comprising the sequence of SEQ ID NO: 123 with a mutation at one or more amino acid positions 27, 55, 56, 57, 58, 105, and/or 106. In some embodiments, the mutation is selected from H27Y, H27D, H27F, H27N, D55E, D55Q, D55N, D55T, S56A, S56Q, S56V, D57S, D57E, D57Q, T58A, T58V, D105E, D105Q, D105T, D105N, D105G, S106A, S106Q, S106V, S106T, or combinations thereof.

In some embodiments, the TREM2 agonist antigen binding protein comprises a light chain variable region comprising the sequence of SEQ ID NO:61 with a mutation at one or more amino acid positions 56, 57, 92, and/or 93. In certain embodiments, the mutation is selected from N56S, N56T, N56Q, N56E, G57A, G57V, D92E, D92Q, D92T, D92N, S93A, S93N, S93Q, S93V, or combinations thereof. In some embodiments, the mutation is selected from N56S, N56Q, G57A, D92E, D92Q, S93A, or combinations thereof. In particular embodiments, the TREM2 agonist antigen binding protein comprises a light chain variable region comprising the sequence of SEQ ID NO:61 with one or more mutations selected from N56S, D92E, and S93A. In these and other embodiments, the TREM2 agonist antigen binding protein comprises a heavy chain variable region comprising the sequence of SEQ ID NO: 124 with a mutation at one or more amino acid positions 55, 56, 57, 58, 105, and/or 106. The mutation can be selected from D55E, D55Q, D55N, D55T, S56A, S56Q, S56V, D57S, D57E, D57Q, T58A, T58V, D105E, D105Q, D105T, D105N, D105G, S106A, S106Q, S106V, S106T, or combinations thereof. In certain embodiments, the mutation is D55E, D55Q, S56A, D57E, T58A, D105E, D105N, S106A, or combinations thereof. In some embodiments, the TREM2 agonist antigen binding protein comprises a heavy chain variable region comprising the sequence of SEQ ID NO: 124 with one or more mutations selected from D55E, S56A, D57E, D105E, and S106A.

In other embodiments, the TREM2 agonist antigen binding protein comprises a light chain variable region comprising the sequence of SEQ ID NO:62 with a mutation at amino acid position 36, 46, 61 and/or 100. In particular embodiments, the mutation is selected from F36Y, S46L, S46R, S46V, S46F, K61R, P100Q, P100G, P100R or combinations thereof. In some embodiments, the mutation is F36Y, K61R, P100Q, or combinations thereof. In some embodiments, the mutation is S46L, P100Q, or combinations thereof. In these and other embodiments, the TREM2 agonist antigen binding protein comprises a heavy chain variable region comprising the sequence of SEQ ID NO: 125 with a mutation at one or more amino acid positions 43, 76, 85, 99, 100, and/or 116. The mutation can be selected from L43Q, L43K, L43H, I76T, R85S, R85G, R85N, R85D, D99E, D99Q, D99S, D99T, G100A, G100Y, G100V, T116L, T116M, T116P, T116R, or combinations thereof. In certain embodiments, the mutation is L43Q, I76T, R85S, D99E, G100A, G100Y, Ti16L, or combinations thereof.

In still other embodiments, the TREM2 agonist antigen binding protein comprises a light chain variable region comprising the sequence of SEQ ID NO:52 with a mutation at amino acid position 91. The mutation can be selected from F91V, F91I, F91T, F91L, or F91D. In one embodiment, the mutation is F91V. In these and other embodiments, the TREM2 agonist antigen binding protein comprises a heavy chain variable region comprising the sequence of SEQ ID NO:115 with a mutation at amino acid position 62 and/or 63. In particular embodiments, the mutation is selected from D62E, D62Q, D62T, D62N, S63A, S63Q, S63V, or combinations thereof. In some embodiments, the mutation is selected from D62E, D62Q, S63A, or combinations thereof.

TABLE A3
Engineered Variants of 10E3 Antibody

| | | | | |
|---|---|---|---|---|
| Position in 10E3 | | | | |
| VL Sequence or | | | Parent Amino | Amino Acid |
| VH sequence | Region | Hot Spot | Acid | Substitutions |

Light chain variable sequence (SEQ ID NO: 54)

| | | | | |
|---|---|---|---|---|
| 64 | FR3 | Covariance violator | V | G, A |
| 79 | FR3 | Covariance violator | Q | E, D |
| 80 | FR3 | Covariance violator | S | P, A |
| 85 | FR3 | Covariance violator | F | V, L, A, D, I, L, M, T |
| 94 | CDR3 | Potential Tryptophan | W | F, Y, S, T, A, H, I, Q |
| | | Oxidation Site | | |
| 100 | FR4 | Covariance violator | P | R, Q, G |

Heavy chain variable sequence (SEQ ID NO: 117)

| | | | | |
|---|---|---|---|---|
| 19 | FR1 | Covariance violator | M | K, R, T, E, N, Q |
| 55-56 | CDR2 | Potential Isomerization Site | DS | ES, QS, DA, NS, DQ, |
| | | | | TS, DV |
| 57-58 | CDR2 | Potential Isomerization Site | DT | ST, ET, DA, DV, QT |
| 104 | CDR3 | Potential Tryptophan | W | F, Y, T, S, A, H, I, Q |
| | | Oxidation Site | | |

TABLE A4
Engineered Variants of 13E7 Antibody

| | | | | |
|---|---|---|---|---|
| Position in 13E7 | | | | |
| VL Sequence or | | | Parent Amino | Amino Acid |
| VH sequence | Region | Hot Spot | Acid | Substitutions |

Light chain variable sequence (SEQ ID NO: 55)

| | | | | |
|---|---|---|---|---|
| 64 | FR3 | Covariance violator | V | G, A |
| 79 | FR3 | Covariance violator | Q | E, D |
| 80 | FR3 | Covariance violator | S | P, A |
| 94 | CDR3 | Potential Tryptophan | W | F, Y, S, T, A, H, I, Q |
| | | Oxidation Site | | |
| 100 | FR4 | Covariance violator | P | R, Q, G |

Heavy chain variable sequence (SEQ ID NO: 118)

| | | | | |
|---|---|---|---|---|
| 19 | FR1 | Covariance violator | M | K, R, T, E, N, Q |
| 55-56 | CDR2 | Potential Isomerization Site | DS | ES, QS, DA, DQ, NS, |
| | | | | TS, DV |
| 57-58 | CDR2 | Potential Isomerization Site | DT | ST, ET, DA, DV, QT |
| 104 | CDR3 | Potential Tryptophan | W | F, Y, T, S, A, H, I, Q |
| | | Oxidation Site | | |

TABLE A5
Engineered Variants of 4C5 Antibody

| | | | | |
|---|---|---|---|---|
| Position in 4C5 | | | | |
| VL Sequence or | | | Parent Amino | Amino Acid |
| VH sequence | Region | Hot Spot | Acid | Substitutions |

Light chain variable sequence (SEQ ID NO: 60)

| | | | | |
|---|---|---|---|---|
| 60 | FR3 | Covariance violator | L | S, P, D, A |
| 92-93 | CDR3 | Potential Isomerization Site | DS | ES, QS, DA, DN, DQ, |
| | | | | TS, NS, DV |

Heavy chain variable sequence (SEQ ID NO: 123)

| | | | | |
|---|---|---|---|---|
| 27 | FR1 | Covariance violator | H | Y, D, F, N |
| 55-56 | CDR2 | Potential Isomerization Site | DS | ES, QS, DA, DQ, DV, |
| | | | | TS, NS |
| 57-58 | CDR2 | Potential Isomerization Site | DT | ST, ET, DA, DV, QT |
| 105-106 | CDR3 | Potential Isomerization Site | DS | ES, QS, DA, DQ, DV, |
| | | | | TS, NS, GT |

TABLE A6
Engineered Variants of 6E7 Antibody

| | | | | |
|---|---|---|---|---|
| Position in 6E7 | | | | |
| VL Sequence or | | | Parent | Amino Acid |
| VH sequence | Region | Hot Spot | Amino Acid | Substitutions |

Light chain variable sequence (SEQ ID NO: 61)

| | | | | |
|---|---|---|---|---|
| 56-57 | CDR2/FR3 | Potential Deamidation Site | NG | SG, TG, QG, NA, EG, |
| | boundary | | | NV |
| 92-93 | CDR3 | Potential Isomerization Site | DS | ES, QS, DA, DN, DQ, |
| | | | | DV, TS, NS |

Heavy chain variable sequence (SEQ ID NO: 124)

| | | | | |
|---|---|---|---|---|
| 55-56 | CDR2 | Potential Isomerization Site | DS | ES, QS, DA, DQ, DV, |
| | | | | TS, NS |
| 57-58 | CDR2 | Potential Isomerization Site | DT | ST, ET, DA, DV, QT |
| 105-106 | CDR3 | Potential Isomerization Site | DS | ES, QS, DA, DQ, DV, |
| | | | | TS, NS, GT |

TABLE A7
Engineered Variants of 5E3 Antibody

| | | | | |
|---|---|---|---|---|
| Position in 5E3 | | | | |
| VL Sequence or | | | Parent | Amino Acid |
| VH sequence | Region | Hot Spot | Amino Acid | Substitutions |

Light chain variable sequence (SEQ ID NO: 62)

| | | | | |
|---|---|---|---|---|
| 36 | FR2 | Consensus violator | F | Y |
| 46 | FR2 | Covariance violator | S | L, R,V, F |
| 61 | FR3 | Consensus violator | K | R |
| 100 | FR4 | Covariance violator | P | Q, G, R |

Heavy chain variable sequence (SEQ ID NO: 125)

| | | | | |
|---|---|---|---|---|
| 43 | FR2 | Covariance violator | L | Q, K, H |
| 76 | FR3 | Covariance violator | I | T |
| 85 | FR3 | Covariance violator | R | S, G, N, D |
| 99-100 | CDR3 | Potential Isomerization Site | DG | EG, DA, DY, DV, |
| | | | | QG, SG, TG |
| 116 | FR4 | Covariance violator | T | L, M, P, R |

TABLE A8
Engineered Variants of 24G6 Antibody

| | | | | |
|---|---|---|---|---|
| Position in 24G6 | | | | |
| VL Sequence or | | | Parent | Amino Acid |
| VH sequence | Region | Hot Spot | Amino Acid | Substitutions |

Light chain variable sequence (SEQ ID NO: 52)

| | | | | |
|---|---|---|---|---|
| 91 | FR3 | Covariance violator | F | V, I, T, L, D |

Heavy chain variable sequence (SEQ ID NO: 115)

| | | | | |
|---|---|---|---|---|
| 62-63 | CDR2 | Potential Isomerization Site | DS | ES, QS, DA, DQ, TS, |
| | | | | DV, NS |

In some embodiments, the TREM2 agonist antigen binding proteins comprise one or more CDRs of a variant of the anti-TREM2 antibodies described herein. In some embodiments, the TREM2 agonist antigen binding proteins may comprise one or more CDRs of the anti-TREM2 antibody variants set forth in TABLES A10, A11, A12, A13, and A14, below.

In certain embodiments, the TREM2 agonist antigen binding proteins of the invention comprise a light chain variable region and/or heavy chain variable region from an affinity-modulated variant of the 6E7 antibody. For instance, in some embodiments, the TREM2 agonist antigen binding proteins comprise a light chain variable region and/or a heavy chain variable region having one or more of the amino acid substitutions set forth in TABLE A9.

TABLE A9
6E7 Antibody Affinity Modulation Variants

| | | | | | | | Binding Signal | | | |
|---|---|---|---|---|---|---|---|---|---|---|
| | | | | | | | (fold over 6E7 | | | |
| | Substitutions with respect | | | | | | Parental antibody) | | | |
| | to 6E7 VH sequence | | | Substitutions with | | | 1^{st} | | | |
| | (SEQ ID NO: 124) | | | respect to 6E7 VL | | | screen | | | |
| | HC | | | (SEQ ID NO: 61) | | | 110 | 2^{nd} | 2^{nd} | 2^{nd} |
| Variant | FR1- | HC | HC | LC | LC | LC | nM or | screen | screen | screen |
| Ab ID | CDR1 | CDR2 | CDR3 | CDR1 | CDR2 | CDR3 | 10 nM^{a} | 2 nM | 10 nM | 100 nM |
| V1 | Y32S | | Q99S | | Q55T | F94Y | 1.68 | 1.29 | 1.92 | |
| V2 | Y27S | S56G | Q99S | | L54R | S93R | 2.55 | 2.23 | 2.90 | |
| V3 | T30A | G66D | Q99G | | L54R | S93R | 1.97 | 1.95 | 2.24 | |
| V4 | T30G | Y60V | Q99S | | S53R | F94Y | 6.00 | 5.88 | 5.51 | |
| V5 | | I50T | | | | F94H | 2.73 | 1.25 | 2.84 | |
| V6 | Y32M | | | | | | 0.20* | | | 0.56 |
| V7 | Y32E | | | | | | 0.11* | | | 0.32 |
| V8 | | R59K | | | | | 0.28* | | | 0.77 |
| V9 | | | T101G | | | | 0.67* | | | 0.54 |
| V10 | | | | | A50S | | 0.76* | | | 0.70 |
| V11 | | | | | | D92A | 0.79* | | | 0.42 |
| V12 | S28E | T58V | Q99G | | N56R | | 2.29 | 1.04 | 2.58 | |
| V13 | T30G | P62A | Q99G | | N56G | F94M | 1.31 | 1.15 | 1.35 | |
| V14 | T30G | S56Q | Q99G | | S53R | | 4.71 | 2.57 | 4.64 | |
| V15 | T30A | I50T | Q99S | | S53W | F94Y | 5.23 | 4.72 | 4.78 | |
| V16 | F29M | S56G | Q99S | | S53N | | 4.01 | 3.57 | 4.04 | |
| V17 | T30G | | Q99S | | L54R | F94S | 5.37 | 4.22 | 5.51 | |
| V18 | W33H | | | | | | 0.17* | | | 0.42 |
| V19 | Y32S | | | | | | 0.59* | | | 0.48 |
| V20 | | I50R | | | | | 0.18* | | | 0.52 |
| V21 | | | Y109F | | | | 0.76* | | | 0.68 |
| V22 | | | | | A50R | | 0.30* | | | 0.71 |
| V23 | | | | | | R96L | 0.40* | | | 0.40 |
| V24 | | T58V | Q99S | | N56K | R96H | 2.64 | 1.42 | 2.90 | |
| V25 | T30G | I50L | Q99S | | Q55A | F94M | 4.23 | 3.15 | 4.70 | |
| V26 | A35G | I50T | F102M, | | N56R | F94Y | 3.57 | 2.83 | 3.47 | |
| | | | Y112A | | | | | | | |
| V27 | | S61A | Q99S | | N56R | | 5.50 | 5.67 | 5.69 | |
| V28 | T30Q | I50T | Y103F | | N56S | F94L | 3.08 | 2.63 | 3.61 | |
| V29 | T30K | | | | | | 1.53 | 0.84 | 1.67 | |
| V30 | Y27S | | | | | | 0.79* | | | 0.72 |
| V31 | | D57E | | | | | 0.61* | | 0.73 | |
| V32 | | P62N | | | | | 0.82* | | | 0.89 |
| V33 | | | Y104G | | | | 0.23* | | | 0.34 |
| V34 | | | | | N56D | | 0.34* | | | 1.02 |
| V35 | | | | | | D92Y | 0.21* | | | 0.29 |
| V36 | I34L | | Q99S | | L54R | F94Y | 3.38 | 4.00 | 3.44 | |
| V37 | F29H | Q65A | Q99S | | N56W | F94Y | 3.46 | 3.69 | 3.49 | |
| V38 | T30G | T58V | | | L54R | F94H | 4.34 | 3.44 | 4.36 | |
| V39 | T30G | S61N | Q99G | | Q55V | F94S | 6.15 | 5.11 | 5.81 | |
| V40 | T30G | T58V | F110S | | N56L | S93R | 4.48 | 3.41 | 4.16 | |
| V41 | | I50T | | | | | 1.74 | 0.58 | 1.72 | |
| V42 | Y32A | | | | | | 0.45* | | | 0.41 |
| V43 | | D57G | | | | | 0.20* | | | 0.33 |
| V44 | | G54S | | | | | 0.65* | | | 0.52 |
| V45 | | | | W32F | | | 0.43* | | | 0.53 |
| V46 | | | | | S53T | | 0.83* | | | 0.96 |
| V47 | | | | | | R96M | 0.42* | | | 0.47 |
| V48 | T30G | T58V | Q99M | | N56T | F94L | 2.42 | 2.30 | 2.54 | |
| V49 | T30N | I50T, | Q99S | | L54R | F94Y | 6.51 | 5.02 | 6.58 | |
| | | Y60L | | | | | | | | |
| V50 | T30G | I50V | F110L | | L54R | F94L | 4.10 | 3.39 | 4.16 | |
| V51 | | T58V | Q99G, | | L54R | | 2.81 | 1.83 | 3.18 | |
| | | | Y112N | | | | | | | |
| V52 | T30E | | Q99G | | N56R | S93R | 3.00 | 1.78 | 3.09 | |
| V53 | | S63H | | | | | 1.25 | 0.66 | 1.17 | |
| V54 | Y32Q | | | | | | 0.55* | | | 0.54 |
| V55 | | R59I, | | | | | 0.24* | | | 0.66 |
| | | F64H | | | | | | | | |
| V56 | | S61Q | | | | | 0.23* | | | 0.59 |
| V57 | | | | R24A | | | 0.84* | | | 0.85 |
| V58 | | | | | A50K | | 0.28* | | | 0.68 |
| V59 | | | | | | Q89M | 0.19* | | | 0.60 |
| V60 | S28H | T58V | F110S | | N56R | Q89G | 3.26 | 3.35 | 3.63 | |
| V61 | T30S | S61N | Q99G | | Q55V | F94L | 5.08 | 3.63 | 5.22 | |
| V62 | T30G | S61A | D108G | | N56R | Q89G | 2.49 | 1.87 | 2.89 | |
| V63 | T30R | | Q99S | | N56R | S93R | 3.76 | 4.91 | 3.71 | |
| V64 | T30Q | | Q99G | | Q55A | F94Y | 5.41 | 4.88 | 5.48 | |
| V65 | | | Q99S | | | | 2.05 | 1.29 | 2.75 | |
| V66 | Y27T | | | | | | 0.25* | | | 0.74 |
| V67 | | I50M | | | | | 0.80* | | | 0.84 |
| V68 | | | Y103R | | | | 0.44* | | | 0.43 |
| V69 | | | | W32Y | | | 0.41* | | | 0.40 |
| V70 | | | | | S52G | | 0.79* | | | 0.84 |
| V71 | | | | | | F94E | 0.37* | | | 0.48 |
| V72 | A35G | | Q99G | | Q55V | F94Y | 3.64 | 2.50 | 4.01 | |
| V73 | T30G | S63G | Q99G | | L54R | F94Y | 5.12 | 4.17 | 5.44 | |
| V74 | T30A | T58V | Q99G | | N56L | | 3.94 | 2.54 | 4.01 | |
| V75 | | | Q99G | | N56A | F94Y | 4.64 | 3.74 | 4.52 | |
| V76 | T30G | S63E | F110S | | N56K | | 4.57 | 4.34 | 4.93 | |
| V77 | | | | | L54R | | 1.43 | 0.83 | 1.38 | |
| V78 | S28R | | | | | | 0.86* | | | 1.11 |
| V79 | | R59N | | | | | 0.70* | | | 0.52 |
| V80 | | | T101N | | | | 0.59* | | | 0.50 |
| V81 | | | | W32L | | | 0.17* | | | 0.23 |
| V82 | | | | | A51G | | 0.30* | | | 0.79 |
| V83 | | | | | | D92V | 0.20* | | | 0.29 |
| V84 | S28G | | F110S | | A50G | | 1.44 | 1.45 | 1.62 | |
| V85 | T30R | I50T | Q99S | | L54R | | 5.41 | 5.41 | 5.37 | |
| V86 | T30G, | Q65E | Q99S | | L54R | | 4.80 | 5.17 | 5.02 | |
| | I34L | | | | | | | | | |
| V87 | T30R | T58V, | Q99S | | N56W | | 3.84 | 4.86 | 3.93 | |
| | | S63D | | | | | | | | |
| V88 | T30G | | | | S53R, | F94S | 4.92 | 5.57 | 5.30 | |
| | | | | | N56R | | | | | |
| V89 | | | | | | F94H | 1.33 | 0.94 | 1.46 | |
| V90 | Y32E | | | S31R | | | 0.33* | | | 0.36 |
| V91 | | G54D | | | | | 0.25* | | | 0.61 |
| V92 | | | Y103H | | | | 0.22* | | | 0.65 |
| V93 | | | | S31G | | | 0.35* | | | 1.05 |
| V94 | | | | | S52A | | 0.31* | | | 0.87 |

In some embodiments, the TREM2 agonist antigen binding protein comprises a light chain variable region comprising the sequence of SEQ ID NO: 61 with a mutation at one or more amino acid positions 24, 31, 50, 52, 54, 56, 89, 92, 93, 94 and/or 96. In certain embodiments, the mutation is selected from R24A, S31R, A50S, A50G, S52G, L54R, N56K, N56R, N56L, N56T, Q89G, D92V, S93R, F94Y, F94L, R96H, R96L, or combinations thereof. In these and other embodiments, the TREM2 agonist antigen binding protein comprises a heavy chain variable region comprising the sequence of SEQ ID NO: 124 with a mutation at one or more amino acid positions 27, 28, 30, 32, 50, 54, 58, 60, 61, 63, 66, 99, 101, 103, 104, and/or 110. In some embodiments, the mutation is selected from Y27S, S28G, S28H, T30N, T30G, T30E, T30A, Y32E, I50T, G54S, T58V, Y60L, S61A, S63G, S63E, G66D, Q99G, Q99S, Q99M, T101G, Y103R, Y104G, F110S, or combinations thereof. Amino acid sequences for light chain and heavy chain variable regions and associated CDRs of exemplary variants of the 6E7 antibody with improved affinity are set forth below in TABLES A7 and A8, respectively. Amino acid sequences for light chain and heavy chain variable regions and associated CDRs of exemplary variants of the 6E7 antibody with reduced affinity are set forth below in TABLES A10 and A11, respectively. The corresponding sequences for the 6E7 antibody are listed for comparison.

TABLE A10
Light Chain Variable Region Amino Acid Sequences for Improved Affinity TREM2 Antibodies

| Variant | VL | | | | |
|---|---|---|---|---|---|
| Ab ID. | Group | VL Amino Acid Sequence | CDRL1 | CDRL2 | CDRL3 |
| 6E7 | LV-16 | DIQMTQSPSSVSASVGDRVTITCRASQGIS | RASQGI | AASSLQ | QQADS |
| | | SWLAWYQQKPGKAPKLLIYAASSLQNGV | SSWLA | N | FPRT |
| | | PSRFSGSGSGTDFTLTISSLQPEDFATYFC | (SEQ ID | (SEQ ID | (SEQ ID |
| | | QQADSFPRTFGQGTKLEIK | NO: 16) | NO: 28) | NO: 43) |
| | | (SEQ ID NO: 61) | | | |
| V3 | LV-101 | DIQMTQSPSSVSASVGDRVTITCRASQGIS | RASQGI | AASSRQ | QQADR |
| | | SWLAWYQQKPGKAPKLLIYAASSRQNGV | SSWLA | N | FPRT |
| | | PSRFSGSGSGTDFTLTISSLQPEDFATYFC | (SEQ ID | (SEQ ID | (SEQ ID |
| | | QQADRFPRTFGQGTKLEIK | NO: 16) | NO: 143) | NO: 148) |
| | | (SEQ ID NO: 153) | | | |
| V24 | LV-102 | DIQMTQSPSSVSASVGDRVTITCRASQGIS | RASQGI | AASSLQ | QQADS |
| | | SWLAWYQQKPGKAPKLLIYAASSLQKGV | SSWLA | K | FPHT |
| | | PSRFSGSGSGTDFTLTISSLQPEDFATYFC | (SEQ ID | (SEQ ID | (SEQ ID |
| | | QQADSFPHTFGQGTKLEIK | NO: 16) | NO: 144) | NO: 149) |
| | | (SEQ ID NO: 154) | | | |
| V27 | LV-103 | DIQMTQSPSSVSASVGDRVTITCRASQGIS | RASQGI | AASSLQ | QQADS |
| | | SWLAWYQQKPGKAPKLLIYAASSLQRGV | SSWLA | R | FPRT |
| | | PSRFSGSGSGTDFTLTISSLQPEDFATYFC | (SEQ ID | (SEQ ID | (SEQ ID |
| | | QQADSFPRTFGQGTKLEIK | NO: 16) | NO: 145) | NO: 43) |
| | | (SEQ ID NO: 155) | | | |
| V40 | LV-104 | DIQMTQSPSSVSASVGDRVTITCRASQGIS | RASQGI | AASSLQ | QQADR |
| | | SWLAWYQQKPGKAPKLLIYAASSLQLGV | SSWLA | L | FPRT |
| | | PSRFSGSGSGTDFTLTISSLQPEDFATYFC | (SEQ ID | (SEQ ID | (SEQ ID |
| | | QQADRFPRTFGQGTKLEIK | NO: 16) | NO: 146) | NO: 148) |
| | | (SEQ ID NO: 156) | | | |
| V48 | LV-105 | DIQMTQSPSSVSASVGDRVTITCRASQGIS | RASQGI | AASSLQ | QQADS |
| | | SWLAWYQQKPGKAPKLLIYAASSLQTGV | SSWLA | T | LPRT |
| | | PSRFSGSGSGTDFTLTISSLQPEDFATYFC | (SEQ ID | (SEQ ID | (SEQ ID |
| | | QQADSLPRTFGQGTKLEIK | NO: 16) | NO: 26) | NO: 150) |
| | | (SEQ ID NO: 157) | | | |
| V49 | LV-106 | DIQMTQSPSSVSASVGDRVTITCRASQGIS | RASQGI | AASSRQ | QQADS |
| V73 | | SWLAWYQQKPGKAPKLLIYAASSRQNGV | SSWLA | N | YPRT |
| | | PSRFSGSGSGTDFTLTISSLQPEDFATYFC | (SEQ ID | (SEQ ID | (SEQ ID |
| | | QQADSYPRTFGQGTKLEIK | NO: 16) | NO: 143) | NO: 151) |
| | | (SEQ ID NO: 158) | | | |
| V52 | LV-107 | DIQMTQSPSSVSASVGDRVTITCRASQGIS | RASQGI | AASSLQ | QQADR |
| | | SWLAWYQQKPGKAPKLLIYAASSLQRGV | SSWLA | R | FPRT |
| | | PSRFSGSGSGTDFTLTISSLQPEDFATYFC | (SEQ ID | (SEQ ID | (SEQ ID |
| | | QQADRFPRTFGQGTKLEIK | NO: 16) | NO: 145) | NO: 148) |
| | | (SEQ ID NO: 159) | | | |
| V60 | LV-108 | DIQMTQSPSSVSASVGDRVTITCRASQGIS | RASQGI | AASSLQ | GQADS |
| | | SWLAWYQQKPGKAPKLLIYAASSLQRGV | SSWLA | R | FPRT |
| | | PSRFSGSGSGTDFTLTISSLQPEDFATYFC | (SEQ ID | (SEQ ID | (SEQ ID |
| | | GQADSFPRTFGQGTKLEIK | NO: 16) | NO: 145) | NO: 152) |
| | | (SEQ ID NO: 160) | | | |
| V76 | LV-109 | DIQMTQSPSSVSASVGDRVTITCRASQGIS | RASQGI | AASSLQ | QQADS |
| | | SWLAWYQQKPGKAPKLLIYAASSLQKGV | SSWLA | K | FPRT |
| | | PSRFSGSGSGRDFTLTISSLQPEDFATYFC | (SEQ ID | (SEQ ID | (SEQ ID |
| | | QQADSFPRTFGQGTKLEIK | NO: 16) | NO: 144) | NO: 43) |
| | | (SEQ ID NO: 161) | | | |
| V84 | LV-110 | DIQMTQSPSSVSASVGDRVTITCRASQGIS | RASQGI | GAS SLQ | QQADS |
| | | SWLAWYQQKPGKAPKLLIYGASSLQNGV | SSWLA | N | FPRT |
| | | PSRFSGSGSGTDFTLTISSLQPEDFATYFC | (SEQ ID | (SEQ ID | (SEQ ID |
| | | QQADSFPRTFGQGTKLEIK | NO: 16) | NO: 147) | NO: 43) |
| | | (SEQ ID NO: 162) | | | |

TABLE A11
Heavy Chain Variable Region Amino Acid Sequences for Improved Affinity TREM2 Antibodies

| | | | FR1/ | | | |
|---|---|---|---|---|---|---|
| Variant | VH | | CDRH1 | | | |
| Ab ID. | Group | VH Amino Acid Sequence | Border | CDRH1 | CDRH2 | CDRH3 |
| 6E7 | HV-15 | EVQLVQSGAEVKKPGESLKIS | YSFT | SYWIA | IIYPGDS | QRTFYY |
| | | CKGSGYSFTSYWIAWVRQMP | (SEQ ID | (SEQ ID | DTRYSP | DSSDYF |
| | | GKGLEWMGITYPGDSDTRYSP | NO: 163) | NO: 85) | SFQG | DY |
| | | SFQGQVTISADKSISTAYLQWS | | | (SEQ ID | (SEQ ID |
| | | SLKASDTAMYFCARQRTFYY | | | NO: 91) | NO: 107) |
| | | DSSDYFDYWGQGTLVTVSS | | | | |
| | | (SEQ ID NO: 124) | | | | |
| V3 | HV-101 | EVQLVQSGAEVKKPGESLKIS | YSFA | SYWIA | IIYPGDS | GRTFYY |
| | | CKGSGYSFASYWIAWVRQMP | (SEQ ID | (SEQ ID | DTRYSP | DSSDYF |
| | | GKGLEWMGITYPGDSDTRYSP | NO: 164) | NO: 85) | SFQD | DY |
| | | SFQDQVTISADKSISTAYLQWS | | | (SEQ ID | (SEQ ID |
| | | SLKASDTAMYFCARGRTFYY | | | NO: 170) | NO: 176) |
| | | DSSDYFDYWGQGTLVTVSS | | | | |
| | | (SEQ ID NO: 180) | | | | |
| V24 | HV-102 | EVQLVQSGAEVKKPGESLKIS | YSFT | SYWIA | IIYPGDS | SRTFYY |
| | | CKGSGYSFTSYWIAWVRQMP | (SEQ ID | (SEQ ID | DVRYSP | DSSDYF |
| | | GKGLEWMGIIYPGDSDVRYSP | NO: 163) | NO: 85) | SFQG | DY |
| | | SFQGQVTISADKSISTAYLQWS | | | (SEQ ID | (SEQ ID |
| | | SLKASDTAMYFCARSRTFYYD | | | NO: 171) | NO: 177) |
| | | SSDYFDYWGQGTLVTVSS | | | | |
| | | (SEQ ID NO: 181) | | | | |
| V27 | HV-103 | EVQLVQSGAEVKKPGESLKIS | YSFT | SYWIA | IIYPGDS | SRTFYY |
| | | CKGSGYSFTSYWIAWVRQMP | (SEQ ID | (SEQ ID | DTRYAP | DSSDYF |
| | | GKGLEWMGIIYPGDSDTRYAP | NO: 163) | NO: 85) | SFQG | DY |
| | | SFQGQVTISADKSISTAYLQWS | | | (SEQ ID | (SEQ ID |
| | | SLKASDTAMYFCVRSRTFYYD | | | NO: 172) | NO: 177) |
| | | SSDYFDYWGQGTLVTVSS | | | | |
| | | (SEQ ID NO: 182) | | | | |
| V40 | HV-104 | EVQLVQSGAEVKKPGESLKIS | YSFG | SYWIA | IIYPGDS | QRTFYY |
| | | CKGSGYSFGSYWIAWVRQMP | (SEQ ID | (SEQ ID | DVRYSP | DSSDYS |
| | | GKGLEWMGIIYPGDSDVRYSP | NO: 165) | NO: 85) | SFQG | DY |
| | | SFQGQVTISADKSISTAYLQWS | | | (SEQ ID | (SEQ ID |
| | | SLKASDTAMYFCARQRTFYY | | | NO: 171) | NO: 178) |
| | | DSSDYSDYWGQGTLVTVSS | | | | |
| | | (SEQ ID NO: 183) | | | | |
| V48 | HV-105 | EVQLVQSGAEVKKPGESLKIS | YSFG | SYWIA | IIYPGDS | MRTFYY |
| | | CKGSGYSFGSYWIAWVRQMP | (SEQ ID | (SEQ ID | DVRYSP | DSSDYF |
| | | GKGLEWMGIIYPGDSDVRYSP | NO: 165) | NO: 85) | SFQG | DY |
| | | SFQGQVTISADKSISTAYLQWS | | | (SEQ ID | (SEQ ID |
| | | SLKASDTAMYFCARMRTFYY | | | NO: 171) | NO: 179) |
| | | DSSDYFDYWGQGTLVTVSS | | | | |
| | | (SEQ ID NO: 184) | | | | |
| V49 | HV-106 | EVQLVQSGAEVKKPGESLKIS | YSFN | SYWIA | TIYPGDS | SRTFYY |
| | | CKGSGYSFNSYWIAWVRQMP | (SEQ ID | (SEQ ID | DTRLSPS | DSSDYF |
| | | GKGLEWMGTIYPGDSDTRLSP | NO: 166) | NO: 85) | FQG | DY |
| | | SFQGQVTISADKSISTAYLQWS | | | (SEQ ID | (SEQ ID |
| | | SLKASDTAMYFCARSRTFYYD | | | NO: 173) | NO: 177) |
| | | SSDYFDYWGQGTLVTVSS | | | | |
| | | (SEQ ID NO: 185) | | | | |
| V52 | HV-107 | EVQLVQSGAEVKKPGESLKIS | YSFE | SYWIA | IIYPGDS | GRTFYY |
| | | CKGSGYSFESYWIAWVRQMP | (SEQ ID | (SEQ ID | DTRYSP | DSSDYF |
| | | GKGLEWMGIIYPGDSDTRYSP | NO: 167) | NO: 85) | SFQG | DY |
| | | SFQGQVTISADKSISTAYLQWS | | | (SEQ ID | (SEQ ID |
| | | SLKASDTAMYFCARGRTFYY | | | NO: 91) | NO: 176) |
| | | DSSDYFDYWGQGTLVTVSS | | | | |
| | | (SEQ ID NO: 186) | | | | |
| V60 | HV-108 | EVQLVQSGAEVKKPGESLKIS | YFIFT | SYWIA | IIYPGDS | QRTFYY |
| | | CKGSGYHFTSYWIAWVRQMP | (SEQ ID | (SEQ ID | DVRYSP | DSSDYS |
| | | GKGLEWMGIIYPGDSDVRYSP | NO: 168) | NO: 85) | SFQG | DY |
| | | SFQGQVTISADKSISTAYLQWS | | | (SEQ ID | (SEQ ID |
| | | SLKASDTAMYFCARQRTFYY | | | NO: 171) | NO: 178) |
| | | DSSDYSDYWGQGTLVTVSS | | | | |
| | | (SEQ ID NO: 187) | | | | |
| V73 | HV-109 | EVQLVQSGAEVKKPGESLKIS | YSFG | SYWIA | IIYPGDS | GRTFYY |
| | | CKGSGYSFGSYWIAWVRQMP | (SEQ ID | (SEQ ID | DTRYSP | DSSDYF |
| | | GKGLEWMGIIYPGDSDTRYSP | NO: 165) | NO: 85) | GFQG | DY |
| | | GFQGQVTISADKSISTAYLQW | | | (SEQ ID | (SEQ ID |
| | | SSLKASDTAMYFCARGRTFYY | | | NO: 174) | NO: 176) |
| | | DSSDYFDYWGQGTLVTVSS | | | | |
| | | (SEQ ID NO: 188) | | | | |
| V76 | HV-110 | EVQLVQSGAEVKKPGESLKIS | YSFG | SYWIA | IIYPGDS | QRTFYY |
| | | CKGSGYSFGSYWIAWVRQMP | (SEQ ID | (SEQ ID | DTRYSP | DSSDYS |
| | | GKGLEWMGITYPGDSDTRYSP | NO: 165) | NO: 85) | EFQG | DY |
| | | EFQGQVTISADKSISTAYLQWS | | | (SEQ ID | (SEQ ID |
| | | SLKASDTAMYFCARQRTFYY | | | NO: 175) | NO: 178) |
| | | DSSDYSDYWGQGTLVTVSS | | | | |
| | | (SEQ ID NO: 189) | | | | |
| V84 | HV-111 | EVQLVQSGAEVKKPGESLKIS | YGFT | SYWIA | IIYPGDS | QRTFYY |
| | | CKGSGYGFTSYWIAWVRQMP | (SEQ ID | (SEQ ID | DTRYSP | DSSDYS |
| | | GKGLEWMGITYPGDSDTRYSP | NO: 169) | NO: 85) | SFQG | DY |
| | | SFQGQVTISADKSISTAYLQWS | | | (SEQ ID | (SEQ ID |
| | | SLKASDTAMYFCARQRTFYY | | | NO: 91) | NO: 178) |
| | | DSSDYSDYWGQGTLVTVSS | | | | |
| | | (SEQ ID NO: 190) | | | | |

In some embodiments, the TREM2 agonist antigen binding proteins of the invention may comprise one or more of the CDRs from the improved affinity variants presented in TABLE A10 (light chain CDRs; i.e. CDRLs) and TABLE A11 (heavy chain CDRs, i.e. CDRHs). In some embodiments, the TREM2 agonist antigen binding proteins comprise a consensus CDR sequence derived from the improved affinity variants. For instance, in some embodiments, the TREM2 agonist antigen binding proteins comprise a CDRL2 consensus sequence of X₁ASSX₂QX₃ (SEQ ID NO: 139), where X₁ is A or G; X₂ is L or R; and X₃ is N, K, R, L, or T. In another embodiment, the TREM2 agonist antigen binding proteins comprise a CDRL3 consensus sequence of X₁QADX_{Z}X₃PX₄T (SEQ ID NO: 140), where X₁ is Q or G; X₂ is S or R; X₃ is F, L, or Y; and X₄ is R or H. In yet another embodiment, the TREM2 agonist antigen binding proteins comprise a CDRH2 consensus sequence of X₁IYPGDSDX_{Z}RX₃X₄PX₅FQX₆ (SEQ ID NO: 141), where X₁ is I or T; X₂ is T or V; X₃ is Y or L; X₄ is S or A; X₅ is S, G, or E; and X₆ is G or D. In some embodiments, the TREM2 agonist antigen binding proteins comprise a CDRH3 consensus sequence of X₁RTFYYDSSDYXzDY (SEQ ID NO: 142), where X₁ is Q, G, S, or M; and X₂ is F or S.

In some embodiments, the TREM2 agonist antigen binding proteins comprise a light chain variable region comprising complementarity determining regions CDRL1, CDRL2, and CDRL3 and a heavy chain variable region comprising complementarity determining regions CDRH1, CDRH2, and CDRH3, wherein CDRL1 comprises the sequence of SEQ ID NO: 16, CDRL2 comprises the consensus sequence of SEQ ID NO: 139, CDRL3 comprises the consensus sequence of SEQ ID NO: 140, CDRH1 comprises the sequence of SEQ ID NO:85, CDRH2 comprises the consensus sequence of SEQ ID NO:141, and CDRH3 comprises the consensus sequence of SEQ ID NO: 142.

In some embodiments, the TREM2 agonist antigen binding protein comprises a CDRL1 comprising the sequence of SEQ ID NO: 16; a CDRL2 comprising a sequence selected from SEQ ID NOS:26 and 143-147; a CDRL3 comprising a sequence selected from SEQ ID NOS:43 and 148-152; a CDRH1 comprising the sequence of SEQ ID NO:85; a CDRH2 comprising a sequence selected from SEQ ID NOS:91 and 170-175; and a CDRH3 comprising a sequence selected from SEQ ID NOS:176-179.

In particular embodiments, the TREM2 agonist antigen binding proteins of the invention comprise a light chain variable region comprising a CDRL1, a CDRL2, and a CDRL3, wherein:
(a) CDRL1, CDRL2, and CDRL3 have the sequence of SEQ ID NOS: 16, 143, and 148, respectively;
(b) CDRL1, CDRL2, and CDRL3 have the sequence of SEQ ID NOS:16, 144, and 149, respectively;
(c) CDRL1, CDRL2, and CDRL3 have the sequence of SEQ ID NOS: 16, 145, and 43, respectively;
(d) CDRL1, CDRL2, and CDRL3 have the sequence of SEQ ID NOS:16, 146, and 148, respectively;
(e) CDRL1, CDRL2, and CDRL3 have the sequence of SEQ ID NOS: 16, 26, and 150, respectively;
(f) CDRL1, CDRL2, and CDRL3 have the sequence of SEQ ID NOS:16, 143, and 151, respectively;
(g) CDRL1, CDRL2, and CDRL3 have the sequence of SEQ ID NOS:16, 145, and 148, respectively;
(h) CDRL1, CDRL2, and CDRL3 have the sequence of SEQ ID NOS:16, 145, and 152, respectively;
(i) CDRL1, CDRL2, and CDRL3 have the sequence of SEQ ID NOS:16, 144, and 43, respectively; or
(j) CDRL1, CDRL2, and CDRL3 have the sequence of SEQ ID NOS:16, 147, and 43, respectively.

In related embodiments, the TREM2 agonist antigen binding proteins of the invention comprise a heavy chain variable region comprising a CDRH1, a CDRH2, and a CDRH3, wherein: (a) CDRH1, CDRH2, and CDRH3 have the sequence of SEQ ID NOS:85, 170, and 176, respectively;
(b) CDRH1, CDRH2, and CDRH3 have the sequence of SEQ ID NOS:85, 171, and 177, respectively;
(c) CDRH1, CDRH2, and CDRH3 have the sequence of SEQ ID NOS:85, 172, and 177, respectively;
(d) CDRH1, CDRH2, and CDRH3 have the sequence of SEQ ID NOS:85, 171, and 178, respectively;
(e) CDRH1, CDRH2, and CDRH3 have the sequence of SEQ ID NOS:85, 171, and 179, respectively;
(f) CDRH1, CDRH2, and CDRH3 have the sequence of SEQ ID NOS:85, 173, and 177, respectively;
(g) CDRH1, CDRH2, and CDRH3 have the sequence of SEQ ID NOS:85, 91, and 176, respectively;
(h) CDRH1, CDRH2, and CDRH3 have the sequence of SEQ ID NOS:85, 174, and 176, respectively;
(i) CDRH1, CDRH2, and CDRH3 have the sequence of SEQ ID NOS:85, 175, and 178, respectively; or
(j) CDRH1, CDRH2, and CDRH3 have the sequence of SEQ ID NOS:85, 91, and 178, respectively.

In some embodiments, the TREM2 agonist antigen binding proteins of the invention comprise a light chain variable region comprising a CDRL1, a CDRL2, and a CDRL3 and a heavy chain variable region comprising a CDRH1, a CDRH2, and a CDRH3, wherein:
(a) CDRL1, CDRL2, and CDRL3 have the sequence of SEQ ID NOS: 16, 143, and 148, respectively, and CDRH1, CDRH2, and CDRH3 have the sequence of SEQ ID NOS:85, 170, and 176, respectively;
(b) CDRL1, CDRL2, and CDRL3 have the sequence of SEQ ID NOS:16, 144, and 149, respectively, and CDRH1, CDRH2, and CDRH3 have the sequence of SEQ ID NOS:85, 171, and 177, respectively;
(c) CDRL1, CDRL2, and CDRL3 have the sequence of SEQ ID NOS: 16, 145, and 43, respectively, and CDRH1, CDRH2, and CDRH3 have the sequence of SEQ ID NOS:85, 172, and 177, respectively;
(d) CDRL1, CDRL2, and CDRL3 have the sequence of SEQ ID NOS:16, 146, and 148, respectively, and CDRH1, CDRH2, and CDRH3 have the sequence of SEQ ID NOS:85, 171, and 178, respectively;
(e) CDRL1, CDRL2, and CDRL3 have the sequence of SEQ ID NOS: 16, 26, and 150, respectively, and CDRH1, CDRH2, and CDRH3 have the sequence of SEQ ID NOS:85, 171, and 179, respectively;
(f) CDRL1, CDRL2, and CDRL3 have the sequence of SEQ ID NOS:16, 143, and 151, respectively, and CDRH1, CDRH2, and CDRH3 have the sequence of SEQ ID NOS:85, 173, and 177, respectively;
(g) CDRL1, CDRL2, and CDRL3 have the sequence of SEQ ID NOS:16, 145, and 148, respectively, and CDRH1, CDRH2, and CDRH3 have the sequence of SEQ ID NOS:85, 91, and 176, respectively;
(h) CDRL1, CDRL2, and CDRL3 have the sequence of SEQ ID NOS:16, 145, and 152, respectively, and CDRH1, CDRH2, and CDRH3 have the sequence of SEQ ID NOS:85, 171, and 178, respectively;
(i) CDRL1, CDRL2, and CDRL3 have the sequence of SEQ ID NOS:16, 143, and 151, respectively, and CDRH1, CDRH2, and CDRH3 have the sequence of SEQ ID NOS:85, 174, and 176, respectively;
(j) CDRL1, CDRL2, and CDRL3 have the sequence of SEQ ID NOS:16, 144, and 43, respectively, and CDRH1, CDRH2, and CDRH3 have the sequence of SEQ ID NOS:85, 175, and 178, respectively; or
(k) CDRL1, CDRL2, and CDRL3 have the sequence of SEQ ID NOS:16, 147, and 43, respectively, and CDRH1, CDRH2, and CDRH3 have the sequence of SEQ ID NOS:85, 91, and 178, respectively.

In some embodiments, the TREM2 agonist antigen binding proteins of the invention may comprise a light chain variable region selected from LV-101, LV-102, LV-103, LV-104, LV-105, LV-106, LV-107, LV-108, LV-109, and LV-110, as shown in TABLE A10, and/or a heavy chain variable region selected from HV-101, HV-102, HV-103, HV-104, HV-105, HV-106, HV-107, HV-108, HV-109, HV-110, and HV-111, as shown in TABLE A11, or sequences that are at least 80% identical, at least 85% identical, at least 90% identical, or at least 95% identical to any of the sequences in TABLE A10 and A11. For instance, in some embodiments, the TREM2 agonist antigen binding proteins comprise a light chain variable region comprising (i) a sequence that is at least 90% identical to a sequence selected from SEQ ID NOS: 153-162, (ii) a sequence that is at least 95% identical to a sequence selected from SEQ ID NOS: 153-162, or (iii) a sequence selected from SEQ ID NOS:153-162. In related embodiments, the TREM2 agonist antigen binding proteins comprise a heavy chain variable region comprising (i) a sequence that is at least 90% identical to a sequence selected from SEQ ID NOS: 180-190, (ii) a sequence that is at least 95% identical to a sequence selected from SEQ ID NOS: 180-190, or (iii) a sequence selected from SEQ ID NOS:180-190.

Each of the light chain variable regions listed in TABLE A10 may be combined with any of the heavy chain variable regions listed in TABLE A11 to form an anti-TREM2 binding domain of the antigen binding proteins of the invention. Examples of such combinations include, but are not limited to: LV-101 (SEQ ID NO:153) and HV-101 (SEQ ID NO:180); LV-102 (SEQ ID NO:154) and HV-102 (SEQ ID NO:181); LV-103 (SEQ ID NO: 155) and HV-103 (SEQ ID NO:182); LV-104 (SEQ ID NO: 156) and HV-104 (SEQ ID NO: 183); LV-105 (SEQ ID NO:157) and HV-105 (SEQ ID NO: 184); LV-106 (SEQ ID NO: 158) and HV-106 (SEQ ID NO:185); LV-107 (SEQ ID NO: 159) and HV-107 (SEQ ID NO: 186); LV-108 (SEQ ID NO:160) and HV-108 (SEQ ID NO: 187); LV-106 (SEQ ID NO: 158) and HV-109 (SEQ ID NO:188); LV-109 (SEQ ID NO: 161) and HV-110 (SEQ ID NO: 189); and LV-110 (SEQ ID NO: 162) and HV-111 (SEQ ID NO: 190).

TABLE A12
Light Chain Variable Region Amino Acid Sequences for Reduced Affinity TREM2 Antibodies

| Variant | VL | | | | |
|---|---|---|---|---|---|
| Ab ID. | Group | VL Amino Acid Sequence | CDRL1 | CDRL2 | CDRL3 |
| 6E7 | LV-16 | DIQMTQSPSSVSASVGDRVTITCRASQGI | RASQGI | AASSLQ | QQADS |
| | | SSWLAWYQQKPGKAPKLLIYAASSLQN | SSWLA | N | FPRT |
| | | GVPSRFSGSGSGTDFTLTISSLQPEDFATY | (SEQ ID | (SEQ ID | (SEQ ID |
| | | FCQQADSFPRTFGQGTKLEIK | NO: 16) | NO: 28) | NO: 43) |
| | | (SEQ ID NO: 61) | | | |
| V9 | LV-16 | DIQMTQSPSSVSASVGDRVTITCRASQGI | RASQGI | AASSLQ | QQADS |
| V30 | | SSWLAWYQQKPGKAPKLLIYAASSLQN | SSWLA | N | FPRT |
| V33 | | GVPSRFSGSGSGTDFTLTISSLQPEDFATY | (SEQ ID | (SEQ ID | (SEQ ID |
| V44 | | FCQQADSFPRTFGQGTKLEIK | NO: 16) | NO: 28) | NO: 43) |
| V68 | | (SEQ ID NO: 61) | | | |
| V10 | LV-201 | DIQMTQSPSSVSASVGDRVTITCRASQGI | RASQGI | SASSLQ | QQADS |
| | | SSWLAWYQQKPGKAPKLLIYSASSLQN | SSWLA | N | FPRT |
| | | GVPSRFSGSGSGTDFTLTISSLQPEDFATY | (SEQ ID | (SEQ ID | (SEQ ID |
| | | FCQQADSFPRTFGQGTKLEIK | NO: 16) | NO: 292) | NO: 43) |
| | | (SEQ ID NO: 295) | | | |
| V23 | LV-202 | DIQMTQSPSSVSASVGDRVTITCRASQGI | RASQGI | AASSLQ | QQADS |
| | | SSWLAWYQQKPGKAPKLLIYAASSLQN | SSWLA | N | FPLT |
| | | GVPSRFSGSGSGTDFTLTISSLQPEDFATY | (SEQ ID | (SEQ ID | (SEQ ID |
| | | FCQQADSFPLTFGQGTKLEIK | NO: 16) | NO: 28) | NO: 294) |
| | | (SEQ ID NO: 296) | | | |
| V57 | LV-203 | DIQMTQSPSSVSASVGDRVTITCAASQGI | AASQGI | AASSLQ | QQADS |
| | | SSWLAWYQQKPGKAPKLLIYAASSLQN | SSWLA | N | FPRT |
| | | GVPSRFSGSGSGTDFTLTISSLQPEDFATY | (SEQ ID | (SEQ ID | (SEQ ID |
| | | FCQQADSFPRTFGQGTKLEIK | NO: 290) | NO: 28) | NO: 43) |
| | | (SEQ ID NO: 297) | | | |
| V70 | LV-204 | DIQMTQSPSSVSASVGDRVTITCRASQGI | RASQGI | AAGSL | QQADS |
| | | SSWLAWYQQKPGKAPKLLIYAAGSLQN | SSWLA | QN | FPRT |
| | | GVPSRFSGSGSGTDFTLTISSLQPEDFATY | (SEQ ID | (SEQ ID | (SEQ ID |
| | | FCQQADSFPRTFGQGTKLEIK | NO: 16) | NO: 293) | NO: 43) |
| | | (SEQ ID NO: 298) | | | |
| V83 | LV-205 | DIQMTQSPSSVSASVGDRVTITCRASQGI | RASQGI | AASSLQ | QQAVS |
| | | SSWLAWYQQKPGKAPKLLIYAASSLQN | SSWLA | N | FPRT |
| | | GVPSRFSGSGSGTDFTLTISSLQPEDFATY | (SEQ ID | (SEQ ID | (SEQ ID |
| | | FCQQAVSFPRTFGQGTKLEIK | NO: 16) | NO: 28) | NO: 271) |
| | | (SEQ ID NO: 299) | | | |
| V90 | LV-206 | DIQMTQSPSSVSASVGDRVTITCRASQGI | RASQGI | AASSLQ | QQADS |
| | | SRWLAWYQQKPGKAPKLLIYAASSLQN | SRWLA | N | FPRT |
| | | GVPSRFSGSGSGTDFTLTISSLQPEDFATY | (SEQ ID | (SEQ ID | (SEQ ID |
| | | FCQQADSFPRTFGQGTKLEIK | NO: 291) | NO: 28) | NO: 43) |
| | | (SEQ ID NO: 300) | | | |

TABLE A13
Heavy Chain Variable Region Amino Acid Sequences for Reduced Affinity TREM2 Antibodies

| Variant | VH | | FR1/CDRH1 | | | |
|---|---|---|---|---|---|---|
| Ab ID. | Group | VH Amino Acid Sequence | border | CDRH1 | CDRH2 | CDRH3 |
| 6E7 | HV-15 | EVQLVQSGAEVKKPGESLKISCK | YSFT | SYWIA | IIYPGD | QRTFYY |
| | | GSGYSFTSYWIAWVRQMPGKG | (SEQ ID | (SEQ ID | SDTRYS | DSSDYF |
| | | LEWMGIIYPGDSDTRYSPSFQGQ | NO: 163) | NO: 85) | PSFQG | DY |
| | | VTISADKSISTAYLQWSSLKASD | | | (SEQ ID | (SEQ ID |
| | | TAMYFCARQRTFYYDSSDYFDY | | | NO: 91) | NO: 107) |
| | | WGQGTLVTVSS | | | | |
| | | (SEQ ID NO: 124) | | | | |
| V9 | HV- | EVQLVQSGAEVKKPGESLKISCK | YSFT | SYWIA | IIYPGD | QRGFYY |
| | 201 | GSGYSFTSYWIAWVRQMPGKG | (SEQ ID | (SEQ ID | SDTRYS | DSSDYF |
| | | LEWMGIIYPGDSDTRYSPSFQGQ | NO: 163) | NO: 85) | PSFQG | DY |
| | | VTISADKSISTAYLQWSSLKASD | | | (SEQ ID | (SEQ ID |
| | | TAMYFCARQRGFYYDSSDYFDY | | | NO: 91) | NO: 304) |
| | | WGQGTLVTVSS | | | | |
| | | (SEQ ID NO: 307) | | | | |
| V10 | HV-15 | EVQLVQSGAEVKKPGESLKISCK | YSFT | SYWIA | IIYPGD | QRTFYY |
| V23 | | GSGYSFTSYWIAWVRQMPGKG | (SEQ ID | (SEQ ID | SDTRYS | DSSDYF |
| V57 | | LEWMGIIYPGDSDTRYSPSFQGQ | NO: 163) | NO: 85) | PSFQG | DY |
| V70 | | VTISADKSISTAYLQWSSLKASD | | | (SEQ ID | (SEQ ID |
| V83 | | TAMYFCARQRTFYYDSSDYFDY | | | NO: 91) | NO: 107) |
| | | WGQGTLVTVSS | | | | |
| | | (SEQ ID NO: 124) | | | | |
| V30 | HV- | EVQLVQSGAEVKKPGESLKISCK | SSFT | SYWIA | IIYPGD | QRTFYY |
| | 202 | GSGSSFTSYWIAWVRQMPGKGL | (SEQ ID | (SEQ ID | SDTRYS | DSSDYF |
| | | EWMGIIYPGDSDTRYSPSFQGQV | NO: 301) | NO: 85) | PSFQG | DY |
| | | TISADKSISTAYLQWSSLKASDT | | | (SEQ ID | (SEQ ID |
| | | AMYFCARQRTFYYDSSDYFDY | | | NO: 91) | NO: 107) |
| | | WGQGTLVTVSS | | | | |
| | | (SEQ ID NO: 308) | | | | |
| V33 | HV- | EVQLVQSGAEVKKPGESLKISCK | YSFT | SYWIA | IIYPGD | QRTFYG |
| | 203 | GSGYSFTSYWIAWVRQMPGKG | (SEQ ID | (SEQ ID | SDTRYS | DSSDYF |
| | | LEWMGIIYPGDSDTRYSPSFQGQ | NO: 163) | NO: 85) | PSFQG | DY |
| | | VTISADKSISTAYLQWSSLKASD | | | (SEQ ID | (SEQ ID |
| | | TAMYFCARQRTFYGDSSDYFDY | | | NO: 91) | NO: 305) |
| | | WGQGTLVTVSS | | | | |
| | | (SEQ ID NO: 309) | | | | |
| V44 | HV- | EVQLVQSGAEVKKPGESLKISCK | YSFT | SYWIA | IIYPSDS | QRTFYY |
| | 204 | GSGYSFTSYWIAWVRQMPGKG | (SEQ ID | (SEQ ID | DTRYSP | DSSDYF |
| | | LEWMGIIYPSDSDTRYSPSFQGQ | NO: 163) | NO: 85) | SFQG | DY |
| | | VTISADKSISTAYLQWSSLKASD | | | (SEQ ID | (SEQ ID |
| | | TAMYFCARQRTFYYDSSDYFDY | | | NO: 303) | NO: 107) |
| | | WGQGTLVTVSS | | | | |
| | | (SEQ ID NO: 310) | | | | |
| V68 | HV- | EVQLVQSGAEVKKPGESLKISCK | YSFT | SYWIA | IIYPGD | QRTFRY |
| | 205 | GSGYSFTSYWIAWVRQMPGKG | (SEQ ID | (SEQ ID | SDTRYS | DSSDYF |
| | | LEWMGIIYPGDSDTRYSPSFQGQ | NO: 163) | NO: 85) | PSFQG | DY |
| | | VTISADKSISTAYLQWSSLKASD | | | (SEQ ID | (SEQ ID |
| | | TAMYFCARQRTFRYDSSDYFDY | | | NO: 91) | NO: 306) |
| | | WGQGTLVTVSS | | | | |
| | | (SEQ ID NO: 311) | | | | |
| V90 | HV- | EVQLVQSGAEVKKPGESLKISCK | YSFT | SEWIA | IIYPGD | QRTFYY |
| | 206 | GSGYSFTSEWIAWVRQMPGKGL | (SEQ ID | (SEQ ID | SDTRYS | DSSDYF |
| | | EWMGIIYPGDSDTRYSPSFQGQV | NO: 163) | NO: 302) | PSFQG | DY |
| | | TISADKSISTAYLQWSSLKASDT | | | (SEQ ID | (SEQ ID |
| | | AMYFCARQRTFYYDSSDYFDY | | | NO: 91) | NO: 107) |
| | | WGQGTLVTVSS | | | | |
| | | (SEQ ID NO: 312) | | | | |

In some embodiments, the TREM2 agonist antigen binding proteins of the invention may comprise one or more of the CDRs from the reduced affinity variants presented in TABLE A12 (light chain CDRs; i.e. CDRLs) and TABLE A13 (heavy chain CDRs, i.e. CDRHs). In some embodiments, the TREM2 agonist antigen binding proteins comprise a consensus CDR sequence derived from the reduced affinity variants. For instance, in one embodiment, the TREM2 agonist antigen binding proteins comprise a CDRL1 consensus sequence of X₁ASQGISX₂WLA (SEQ ID NO:284), where X₁ is R or A; and X₂ is S or R. In another embodiment, the TREM2 agonist antigen binding proteins comprise a CDRL2 consensus sequence of X₁AX₂SLQN (SEQ ID NO:285), where X₁ is A or S; and X₂ is S or G. In another embodiment, the TREM2 agonist antigen binding proteins comprise a CDRL3 consensus sequence of QQAX₁SFPX₂T (SEQ ID NO:286), where X₁ is D or V; and X₂ is R or L. In another embodiment, the TREM2 agonist antigen binding proteins comprise a CDRH1 consensus sequence of SX₁WIA (SEQ ID NO:287), where X₁ is Y or E. In yet another embodiment, the TREM2 agonist antigen binding proteins comprise a CDRH2 consensus sequence of IIYPX₁DSDTRYSPSFQG (SEQ ID NO:288), where X₁ is G or S. In still another embodiment, the TREM2 agonist antigen binding proteins comprise a CDRH3 consensus sequence of QRX₁FX₂X₃DSSDYFDY (SEQ ID NO:289), where X₁ is T or G; X₂ is Y or R; and X₃ is Y or G. In some embodiments, the TREM2 agonist antigen binding proteins comprise a light chain variable region comprising complementarity determining regions CDRL1, CDRL2, and CDRL3 and a heavy chain variable region comprising complementarity determining regions CDRH1, CDRH2, and CDRH3, wherein CDRL1 comprises the sequence of SEQ ID NO:284, CDRL2 comprises the consensus sequence of SEQ ID NO:285, CDRL3 comprises the consensus sequence of SEQ ID NO:286, CDRH1 comprises the sequence of SEQ ID NO:287, CDRH2 comprises the consensus sequence of SEQ ID NO:288, and CDRH3 comprises the consensus sequence of SEQ ID NO:289.

In some embodiments, the TREM2 agonist antigen binding proteins of the invention comprise a CDRL1 comprising a sequence selected from SEQ ID NOS:16, 290, and 291; a CDRL2 comprising a sequence selected from SEQ ID NOS:28, 292, and 293; a CDRL3 comprising a sequence selected from SEQ ID NOS:43, 294, and 271; a CDRH1 comprising the sequence of SEQ ID NO:85 or SEQ ID NO:302; a CDRH2 comprising the sequence of SEQ ID NO:91 or SEQ ID NO:303; and a CDRH3 comprising a sequence selected from SEQ ID NOS:107 and 304-306.

In some embodiments, the TREM2 agonist antigen binding proteins of the invention comprise a light chain variable region comprising a CDRL1, a CDRL2, and a CDRL3, wherein: (a) CDRL1, CDRL2, and CDRL3 have the sequence of SEQ ID NOS:16, 28, and 43, respectively;
(b) CDRL1, CDRL2, and CDRL3 have the sequence of SEQ ID NOS:16, 292, and 43, respectively;
(c) CDRL1, CDRL2, and CDRL3 have the sequence of SEQ ID NOS: 16, 28, and 294, respectively;
(d) CDRL1, CDRL2, and CDRL3 have the sequence of SEQ ID NOS:290, 28, and 43, respectively;
(e) CDRL1, CDRL2, and CDRL3 have the sequence of SEQ ID NOS: 16, 293, and 43, respectively;
(f) CDRL1, CDRL2, and CDRL3 have the sequence of SEQ ID NOS:16, 28, and 271, respectively; or
(g) CDRL1, CDRL2, and CDRL3 have the sequence of SEQ ID NOS:291, 28, and 43, respectively.

In related embodiments, the TREM2 agonist antigen binding proteins of the invention comprise a heavy chain variable region comprising a CDRH1, a CDRH2, and a CDRH3, wherein:
(a) CDRH1, CDRH2, and CDRH3 have the sequence of SEQ ID NOS:85, 91, and 304, respectively;
(b) CDRH1, CDRH2, and CDRH3 have the sequence of SEQ ID NOS:85, 91, and 107, respectively;
(c) CDRH1, CDRH2, and CDRH3 have the sequence of SEQ ID NOS:85, 91, and 305, respectively;
(d) CDRH1, CDRH2, and CDRH3 have the sequence of SEQ ID NOS:85, 303, and 107, respectively;
(e) CDRH1, CDRH2, and CDRH3 have the sequence of SEQ ID NOS:85, 91, and 306, respectively; or
(f) CDRH1, CDRH2, and CDRH3 have the sequence of SEQ ID NOS:302, 91, and 107, respectively.

In some embodiments, the TREM2 agonist antigen binding proteins of the invention comprise a light chain variable region comprising a CDRL1, a CDRL2, and a CDRL3 and a heavy chain variable region comprising a CDRH1, a CDRH2, and a CDRH3, wherein:
(a) CDRL1, CDRL2, and CDRL3 have the sequence of SEQ ID NOS: 16, 28, and 43, respectively, and CDRH1, CDRH2, and CDRH3 have the sequence of SEQ ID NOS:85, 91, and 304, respectively;
(b) CDRL1, CDRL2, and CDRL3 have the sequence of SEQ ID NOS:16, 292, and 43, respectively, and CDRH1, CDRH2, and CDRH3 have the sequence of SEQ ID NOS:85, 91, and 107, respectively;
(c) CDRL1, CDRL2, and CDRL3 have the sequence of SEQ ID NOS: 16, 28, and 294, respectively, and CDRH1, CDRH2, and CDRH3 have the sequence of SEQ ID NOS:85, 91, and 107, respectively;
(d) CDRL1, CDRL2, and CDRL3 have the sequence of SEQ ID NOS: 16, 28, and 43, respectively, and CDRH1, CDRH2, and CDRH3 have the sequence of SEQ ID NOS:85, 91, and 107, respectively;
(e) CDRL1, CDRL2, and CDRL3 have the sequence of SEQ ID NOS: 16, 28, and 43, respectively, and CDRH1, CDRH2, and CDRH3 have the sequence of SEQ ID NOS:85, 91, and 305, respectively;
(f) CDRL1, CDRL2, and CDRL3 have the sequence of SEQ ID NOS: 16, 28, and 43, respectively, and CDRH1, CDRH2, and CDRH3 have the sequence of SEQ ID NOS:85, 303, and 107, respectively;
(g) CDRL1, CDRL2, and CDRL3 have the sequence of SEQ ID NOS:290, 28, and 43, respectively, and CDRH1, CDRH2, and CDRH3 have the sequence of SEQ ID NOS:85, 91, and 107, respectively;
(h) CDRL1, CDRL2, and CDRL3 have the sequence of SEQ ID NOS: 16, 28, and 43, respectively, and CDRH1, CDRH2, and CDRH3 have the sequence of SEQ ID NOS:85, 91, and 306, respectively;
(i) CDRL1, CDRL2, and CDRL3 have the sequence of SEQ ID NOS:16, 293, and 43, respectively, and CDRH1, CDRH2, and CDRH3 have the sequence of SEQ ID NOS:85, 91, and 107, respectively;
(j) CDRL1, CDRL2, and CDRL3 have the sequence of SEQ ID NOS:16, 28, and 271, respectively, and CDRH1, CDRH2, and CDRH3 have the sequence of SEQ ID NOS:85, 91, and 107, respectively; or
(k) CDRL1, CDRL2, and CDRL3 have the sequence of SEQ ID NOS:291, 28, and 43, respectively, and CDRH1, CDRH2, and CDRH3 have the sequence of SEQ ID NOS:302, 91, and 107, respectively.

In some embodiments, the TREM2 agonist antigen binding proteins of the invention may comprise a light chain variable region selected from LV-16, LV-201, LV-202, LV-203, LV-204, LV-205, and LV-206, as shown in TABLE A12, and/or a heavy chain variable region selected from HV-15, HV-201, HV-202, HV-203, HV-204, HV-205, and HV-206, as shown in TABLE A13, or sequences that are at least 80% identical, at least 85% identical, at least 90% identical, or at least 95% identical to any of the sequences in TABLES A12 and A13. For instance, in certain embodiments, the TREM2 agonist antigen binding proteins comprise a light chain variable region comprising (i) a sequence that is at least 90% identical to a sequence selected from SEQ ID NOS:61 and 295-300, (ii) a sequence that is at least 95% identical to a sequence selected from SEQ ID NOS:61 and 295-300, or (iii) a sequence selected from SEQ ID NOS:61 and 295-300. In related embodiments, the TREM2 agonist antigen binding proteins comprise a heavy chain variable region comprising (i) a sequence that is at least 90% identical to a sequence selected from SEQ ID NOS:124 and 307-312, (ii) a sequence that is at least 95% identical to a sequence selected from SEQ ID NOS:124 and 307-312, or (iii) a sequence selected from SEQ ID NOS: 124 and 307-312.

In some embodiments, each of the light chain variable regions listed in TABLE A12 may be combined with any of the heavy chain variable regions listed in TABLE A13 to form an anti-TREM2 binding domain of the antigen binding proteins of the invention. Examples of such combinations include, but are not limited to: LV-16 (SEQ ID NO:61) and HV-201 (SEQ ID NO:307); LV-201 (SEQ ID NO:295) and HV-15 (SEQ ID NO: 124); LV-202 (SEQ ID NO:296) and HV-15 (SEQ ID NO: 124); LV-16 (SEQ ID NO:61) and HV-202 (SEQ ID NO:308); LV-16 (SEQ ID NO:61) and HV-203 (SEQ ID NO:309); LV-16 (SEQ ID NO:61) and HV-204 (SEQ ID NO:310); LV-203 (SEQ ID NO:297) and HV-15 (SEQ ID NO: 124); LV-16 (SEQ ID NO:61) and HV-205 (SEQ ID NO:311); LV-204 (SEQ ID NO:298) and HV-15 (SEQ ID NO: 124); LV-205 (SEQ ID NO:299) and HV-15 (SEQ ID NO: 124); and LV-206 (SEQ ID NO:300) and HV-206 (SEQ ID NO:312).

In some embodiments, the TREM2 agonist antigen binding proteins comprise one or more CDRs of the anti-TREM2 antibody variants set forth in TABLE A14. In some embodiments, the TREM2 agonist antigen binding proteins comprise the light chain variable region and heavy chain variable region of the anti-TREM2 antibody variants set forth in TABLE A14.

TABLE A14
Exemplary Variable Region Amino Acid Sequences of Engineered Antibodies

| Ab ID. | LC variable region | CDRL1 | CDRL2 | CDRL3 |
|---|---|---|---|---|
| 24G6 | DIVMTQSPDSLAVSLGERATINCKSSQSVLY | KSSQSV | WASTR | QQYYS |
| (SST28347 | SSNNKHFLAWYQQKPGQPPKLLIYWASTRLYSSN | LYSSNN | ES | TPLT |
| and | ESGVPDRFSGSGSGTDFTLTISSLQAEDVAV | KHFLA | (SEQ ID | (SEQ ID |
| SST204812) | YYCQQYYSTPLTFGGGTKVEIK | NO: 8) | NO: 22) | NO: 35) |
| | (SEQ ID NO: 326) | | | |
| 6E7 | DIQMTQSPSSVSASVGDRVTITCRASQGISS | RASQGI | AASSLQ | QQADA |
| (SST29857) | WLAWYQQKPGKAPKLLIYAASSLQSGVPS | SSWLA | S | FPRT |
| | RFSGSGSGTDFTLTISSLQPEDFATYFCQQA | (SEQ ID | (SEQ ID | (SEQ ID |
| | DAFPRTFGQGTKLEIK (SEQ ID NO: 328) | NO: 16) | NO: 369) | NO: 370) |
| 13E7 | EIVMTQSPATLSVSPGERATLSCRASQSVSS | RASQS | GASTR | LQDNN |
| (SST202443) | NLAWFQQKPGQAPRLLIYGASTRATGIPAR | VSSNLA | AT | FPPT |
| | FSGSGSGTEFTLTISSLQPEDFAVYYCLQDN | (SEQ ID | (SEQ ID | (SEQ ID |
| | NFPPTFGQGTKVDIK (SEQ ID NO: 330) | NO: 10) | NO: 23) | NO: 372) |
| 5E3 | DIQMTQSPSSLSASVGDRVTITCRASQGISN | RASQGI | AASSLQ | QQYST |
| (SST29825) | YLAWYQQKPGKAPKSLIYAASSLQSGVPSR | SNYLA | S | YPFT |
| | FSGSGSGTDFTLTISSLQPEDFATYYCQQYS | (SEQ ID | (SEQ ID | (SEQ ID |
| | TYPFTFGQGTKVDIK (SEQ ID NO: 332) | NO: 17) | NO: 29) | NO: 44) |
| Ab ID. | HC variable region | CDRH1 | CDRH2 | CDRH3 |
| 24G6 | EVQLLESGGGLVQPGGSLRLSCAASGFTFS | SYAMS | AISGSG | AYTPM |
| (SST28347 | SYAMSWVRQAPGKGLEWVSAISGSGGSTY | (SEQ ID | GSTYY | AFFDY |
| and | YAESVKGRFTISRDNSKNTLYLQMNSLRAE | NO: 77) | AESVK | (SEQ ID |
| SST204812) | DTAVYYCAKAYTPMAFFDYWGQGTLVTV | | G | NO: 98) |
| | SS (SEQ ID NO: 327) | | (SEQ ID | |
| | | | NO: 368) | |
| 6E7 | EVQLVQSGAEVKKPGESLKISCKGSGYSFT | SYWIA | IIYPGD | QRTFY |
| (SST29857) | SYWIAWVRQMPGKGLEWMGITYPGDADA | (SEQ ID | ADARY | YDSSD |
| | RYSPSFQGQVTISADKSISTAYLQWSSLKAS | NO: 85) | SPSFQG | YFDY |
| | DTAMYFCARQRTFYYDSSDYFDYWGQGT | | (SEQ ID | (SEQ ID |
| | LVTVSS (SEQ ID NO: 329) | | NO: 371) | NO: 107) |
| 13E7 | EVQLVQSGAEVKKPGESLKISCKGSGYSFT | SWIG | IIYPGD | RRQGIF |
| (SST202443) | SYWIGWVRQMPGKGLEWMGITYPGDADA | (SEQ ID | ADARY | GDALD |
| | RYSPSFQGQVTISADKSISTAYLQWSSLKAS | NO: 81) | SPSFQG | F |
| | DTAMYFCARRRQGIFGDALDFWGQGTLVT | | (SEQ ID | (SEQ ID |
| | VSS (SEQ ID NO: 331) | | NO: 373) | NO: 374) |
| | QVQLVQSGAEVKKPGASVKVSCKASGYTF | GYYIH | WINPYS | DAGYL |
| | TGYYIHWVRQAPGQGLEWMGWINPYSGG | (SEQ ID | GGTTS | ALYGT |
| | TTSAQKFQGRVTMTRDTSTSSAYMELSRLR | NO: 86) | AQKFQ | DV |
| | SDDTAVYYCARDAGYLALYGTDVWGQGT | | G | (SEQ ID |
| | LVTVSS (SEQ ID NO: 333) | | (SEQ ID | NO: 375 |
| | | | NO: 94 | |

In some embodiments, the TREM2 agonist antigen binding protein comprises a light chain variable region comprising a CDRL1, a CDRL2, and a CDRL3, wherein:
(a) CDRL1, CDRL2, and CDRL3 have the sequence of SEQ ID NOS: 16, 369, and 370, respectively;
(b) CDRL1, CDRL2, and CDRL3 have the sequence of SEQ ID NOS: 10, 23, and 372, respectively; or
(c) CDRL1, CDRL2, and CDRL3 have the sequence of SEQ ID NOS:6, 21, and 33, respectively;
(d) CDRL1, CDRL2, and CDRL3 have the sequence of SEQ ID NOS:6, 20, and 33, respectively.

In some embodiments, the TREM2 agonist antigen binding protein comprises a heavy chain variable region comprising a CDRH1, a CDRH2, and a CDRH3, wherein:
(a) CDRH1, CDRH2, and CDRH3 have the sequence of SEQ ID NOS:77, 368, and 98, respectively;
(b) CDRH1, CDRH2, and CDRH3 have the sequence of SEQ ID NOS:85, 371, and 107, respectively;
(c) CDRH1, CDRH2, and CDRH3 have the sequence of SEQ ID NOS:81, 373, and 374, respectively; or
(d) CDRH1, CDRH2, and CDRH3 have the sequence of SEQ ID NOS:86, 94, and 375, respectively.

In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein:
(a) CDRL1, CDRL2, and CDRL3 have the sequence of SEQ ID NOS:8, 22, and 35, respectively, and CDRH1, CDRH2, and CDRH3 have the sequence of SEQ ID NOS:77, 368, and 98, respectively;
(b) CDRL1, CDRL2, and CDRL3 have the sequence of SEQ ID NOS:16, 369, and 370, respectively, and CDRH1, CDRH2, and CDRH3 have the sequence of SEQ ID NOS:85, 371, and 107, respectively;
(c) CDRL1, CDRL2, and CDRL3 have the sequence of SEQ ID NOS: 10, 23, and 372, respectively, and CDRH1, CDRH2, and CDRH3 have the sequence of SEQ ID NOS:81, 373, and 374, respectively; or
(d) CDRL1, CDRL2, and CDRL3 have the sequence of SEQ ID NOS: 17, 29, and 44, respectively, and CDRH1, CDRH2, and CDRH3 have the sequence of SEQ ID NOS:86, 94, and 375, respectively.

Accordingly, in some embodiments, the TREM2 agonist antigen binding protein comprises a light chain variable region comprising a CDRL1, a CDRL2, and a CDRL3, and a heavy chain variable region comprising a CDRH1, a CDRH2, and a CDRH3, wherein the CDRL1, CDRL2, and CDRL3 have the sequence of SEQ ID NOS: 10, 23, and 372, respectively, and the CDRH1, CDRH2, and CDRH3 have the sequence of SEQ ID NOS:81, 373, and 374, respectively.

In some embodiments therefore, the present invention provides a method of treating ALSP in a human patient, the method comprising administering to the patient an effective amount of a TREM2 agonist antigen binding protein comprising a CDRL1, CDRL2, and CDRL3 having the sequence of SEQ ID NOS:10, 23, and 372, respectively, and a CDRH1, CDRH2, and CDRH3 having the sequence of SEQ ID NOS:81, 373, and 374, respectively. In certain embodiments, the antibody is human. In some embodiments, the TREM2 agonist antigen binding protein comprises
(a) a light chain variable region comprising the amino acid sequence of SEQ ID NO:326 and a heavy chain variable region comprising the amino acid sequence of SEQ ID NO:327;
(b) a light chain variable region comprising the amino acid sequence of SEQ ID NO:328 and a heavy chain variable region comprising the amino acid sequence of SEQ ID NO:329;
(c) a light chain variable region comprising the amino acid sequence of SEQ ID NO:330 and a heavy chain variable region comprising the amino acid sequence of SEQ ID NO:331; or
(d) a light chain variable region comprising the amino acid sequence of SEQ ID NO:332 and a heavy chain variable region comprising the amino acid sequence of SEQ ID NO:333.

In some embodiments, the TREM2 agonist antigen binding protein comprises a light chain variable region comprising the amino acid sequence of SEQ ID NO:330 and a heavy chain variable region comprising the amino acid sequence of SEQ ID NO:331.

In some embodiments therefore, the present invention provides a method of treating ALSP in a human patient, the method comprising administering to the patient an effective amount of a TREM2 agonist antigen binding protein comprising a light chain variable region comprising the amino acid sequence of SEQ ID NO:330 and a heavy chain variable region comprising the amino acid sequence of SEQ ID NO:331. In certain embodiments, the antibody is human.

In some embodiments, the TREM2 agonist antigen binding proteins of the invention comprise a light chain variable region consisting of or consisting essentially of the amino acid sequence of SEQ ID NO:326, 328, 330 or 332. In some embodiments, the TREM2 agonist antigen binding proteins of the invention comprise a heavy chain variable region consisting of or consisting essentially of the amino acid sequence of SEQ ID NO:327, 329, 331 or 333. In a specific embodiment, the TREM2 agonist antigen binding proteins of the invention comprise a light chain variable region and a heavy chain variable region, wherein the light chain variable region consisting of or consisting essentially of the amino acid sequence of SEQ ID NO:326 and the heavy chain variable region consisting of or consisting essentially of the amino acid sequence of SEQ ID NO:327. In a specific embodiment, the TREM2 agonist antigen binding proteins of the invention comprise a light chain variable region and a heavy chain variable region, wherein the light chain variable region consisting of or consisting essentially of the amino acid sequence of SEQ ID NO:328 and the heavy chain variable region consisting of or consisting essentially of the amino acid sequence of SEQ ID NO:329. In a specific embodiment, the TREM2 agonist antigen binding proteins of the invention comprise a light chain variable region and a heavy chain variable region, wherein the light chain variable region consisting of or consisting essentially of the amino acid sequence of SEQ ID NO:330 and the heavy chain variable region consisting of or consisting essentially of the amino acid sequence of SEQ ID NO:331. In a specific embodiment, the TREM2 agonist antigen binding proteins of the invention comprise a light chain variable region and a heavy chain variable region, wherein the light chain variable region consisting of or consisting essentially of the amino acid sequence of SEQ ID NO:332 and the heavy chain variable region consisting of or consisting essentially of the amino acid sequence of SEQ ID NO:333.

In some embodiments, each of the light chain variable regions disclosed in TABLES A1, A10, A12, and A14 and each of the heavy chain variable regions disclosed in TABLES A2, A11, A13, and 3E may be attached to the light chain constant regions (TABLE EN1) and heavy chain constant regions (EN2) to form complete antibody light and heavy chains, respectively, as further discussed below. Further, each of the generated heavy and light chain sequences may be combined to form a complete antibody structure. It should be understood that the heavy chain and light chain variable regions provided herein can also be attached to other constant domains having different sequences than the exemplary sequences listed herein.

In some embodiments, exemplary TREM2 agonist antibody having a light chain variable region with a light chain constant domain and a heavy chain variable region with a heavy chain constant region are disclosed in TABLE A15.

TABLE A15
Light Chain and Heavy Chain Amino Acid Sequences
of Exemplary Antibodies

| Ab ID. | | Sequence |
|---|---|---|
| 24G6 | LC | MDMRVPAQLLGLLLLWLRGARCDIVMTQSPDSLAVSLGERATIN |
| (SST28347) | | CKSSQSVLYSSNNKHFLAWYQQKPGQPPKLLIYWASTRESGVPD |
| | | RFSGSGSGTDFTLTISSLQAEDVAVYYCQQYYSTPLTFGGGTKVE |
| | | IKRTVAAPSVFIFPPSDEQLKSGTASVVCLLNNFYPREAKVQWKV |
| | | DNALQSGNSQESVTEQDSKDSTYSLSSTLTLSKADYEKHKVYAC |
| | | EVTHQGLSSPVTKSFNRGEC (SEQ ID NO: 334) |
| | HC | MDMRVPAQLLGLLLLWLRGARCEVQLLESGGGLVQPGGSLRLS |
| | | CAASGFTFSSYAMSWVRQAPGKGLEWVSAISGSGGSTYYAESVK |
| | | GRFTISRDNSKNTLYLQMNSLRAEDTAVYYCAKAYTPMAFFDY |
| | | WGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQ |
| | | TYICNVNHKPSNTKVDKKVEPKSCDKTHTCPPCPAPELLGGPSVF |
| | | LFPPKPKDTLMISRTPEVTCVVVDVSHEDPEVKFNWYVDGVEVH |
| | | NAKTKPCEEQYGSTYRCVSVLTVLHQDWLNGKEYKCKVSNKAL |
| | | PAPIEKTISKAKGQPREPQVYTLPPSREEMTKNQVSLTCLVKGFY |
| | | PSDIAVEWESNGQPENNYKTTPPVLDSDGSFFLYSKLTVDKSRW |
| | | QQGNVFSCSVMHEALHNHYTQKSLSLSPGK (SEQ ID NO: 335) |
| 24G6 | LC | MDMRVPAQLLGLLLLWLRGARCDIVMTQSPDSLAVSLGERATIN |
| (SST204812) | | CKSSQSVLYSSNNKHFLAWYQQKPGQPPKLLIYWASTRESGVPD |
| | | RFSGSGSGTDFTLTISSLQAEDVAVYYCQQYYSTPLTFGGGTKVE |
| | | IKRTVAAPSVFIFPPSDEQLKSGTASVVCLLNNFYPREAKVQWKV |
| | | DNALQSGNSQESVTEQDSKDSTYSLSSTLTLSKADYEKHKVYAC |
| | | EVTHQGLSSPVTKSFNRGEC (SEQ ID NO: 334) |
| | HC | MDMRVPAQLLGLLLLWLRGARCEVQLLESGGGLVQPGGSLRLS |
| | | CAASGFTFSSYAMSWVRQAPGKGLEWVSAISGSGGSTYYAESVK |
| | | GRFTISRDNSKNTLYLQMNSLRAEDTAVYYCAKAYTPMAFFDY |
| | | WGQGTLVTVSSASTKGPSVFPLAPSSRSTSESTAALGCLVKDYFP |
| | | EPVTVSWNSGALTSGVHTFPAVLQSSGLYSLSSVVTVPSSNFGTQ |
| | | TYTCNVDHKPSNTKVDKTVERKCCVECPPCPAPELLGGPSVFLFP |
| | | PKPKDTLMISRTPEVTCVVVDVSHEDPEVKFNWYVDGVEVHNA |
| | | KTKPCEEQYGSTYRCVSVLTVLHQDWLNGKEYKCKVSNKALPA |
| | | PIEKTISKAKGQPREPQVYTLPPSREEMTKNQVSLTCLVKGFYPS |
| | | DIAVEWESNGQPENNYKTTPPVLDSDGSFFLYSKLTVDKSRWQQ |
| | | GNVFSCSVMHEALHNHYTQKSLSLSPGK (SEQ ID NO: 336) |
| 6E7 | LC | MDMRVPAQLLGLLLLWLRGARCDIQMTQSPSSVSASVGDRVTIT |
| (SST29857) | | CRASQGISSWLAWYQQKPGKAPKLLIYAASSLQSGVPSRFSGSGS |
| | | GTDFTLTISSLQPEDFATYFCQQADAFPRTFGQGTKLEIKRTVAAP |
| | | SVFIFPPSDEQLKSGTASVVCLLNNFYPREAKVQWKVDNALQSG |
| | | NSQESVTEQDSKDSTYSLSSTLTLSKADYEKHKVYACEVTHQGL |
| | | SSPVTKSFNRGEC (SEQ ID NO: 337) |
| | HC | MDMRVPAQLLGLLLLWLRGARCEVQLVQSGAEVKKPGESLKIS |
| | | CKGSGYSFTSYWIAWVRQMPGKGLEWMGITYPGDADARYSPSF |
| | | QGQVTISADKSISTAYLQWSSLKASDTAMYFCARQRTFYYDSSD |
| | | YFDYWGQGTLVTVSSASTKGPSVFPLAPSSRSTSESTAALGCLVK |
| | | DYFPEPVTVSWNSGALTSGVHTFPAVLQSSGLYSLSSVVTVPSSN |
| | | FGTQTYTCNVDHKPSNTKVDKTVERKCCVECPPCPAPELLGGPS |
| | | VFLFPPKPKDTLMISRTPEVTCVVVDVSHEDPEVKFNWYVDGVE |
| | | VHNAKTKPCEEQYGSTYRCVSVLTVLHQDWLNGKEYKCKVSNK |
| | | ALPAPIEKTISKAKGQPREPQVYTLPPSREEMTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK (SEQ ID NO: 338) |
| 13E7 | LC | MDMRVPAQLLGLLLLWLRGARCEIVMTQSPATLSVSPGERATLS |
| (SST202443) | | CRASQSVSSNLAWFQQKPGQAPRLLIYGASTRATGIPARFSGSGS |
| | | GTEFTLTISSLQPEDFAVYYCLQDNNFPPTFGQGTKVDIKRTVAA |
| | | PSVFIFPPSDEQLKSGTASVVCLLNNFYPREAKVQWKVDNALQS |
| | | GNSQESVTEQDSKDSTYSLSSTLTLSKADYEKHKVYACEVTHQG |
| | | LSSPVTKSFNRGEC (SEQ ID NO: 339) |
| | HC | MDMRVPAQLLGLLLLWLRGARCEVQLVQSGAEVKKPGESLKIS |
| | | CKGSGYSFTSYWIGWVRQMPGKGLEWMGITYPGDADARYSPSF |
| | | QGQVTISADKSISTAYLQWSSLKASDTAMYFCARRRQGIFGDAL |
| | | DFWGQGTLVTVSSASTKGPSVFPLAPSSKSTSGGTAALGCLVKD |
| | | YFPEPVTVSWNSGALTSGVHTFPAVLQSSGLYSLSSVVTVPSSSL |
| | | GTQTYICNVNHKPSNTKVDKKVEPKSCDKTHTCPPCPAPELLGG |
| | | PSVFLFPPKPKDTLMISRTPEVTCVVVDVSHEDPEVKFNWYVDG |
| | | VEVHNAKTKPCEEQYGSTYRCVSVLTVLHQDWLNGKEYKCKVS |
| | | NKALPAPIEKTISKAKGQPREPQVYTLPPSREEMTKNQVSLTCLV |
| | | KGFYPSDIAVEWESNGQPENNYKTTPPVLDSDGSFFLYSKLTVDK |
| | | SRWQQGNVFSCSVMHEALHNHYTQKSLSLSPGK |
| | | (SEQ ID NO: 340) |
| 5E3 (SST29825) | LC | MDMRVPAQLLGLLLLWLRGARCDIQMTQSPSSLSASVGDRVTIT |
| | | CRASQGISNYLAWYQQKPGKAPKSLIYAASSLQSGVPSRFSGSGS |
| | | GTDFTLTISSLQPEDFATYYCQQYSTYPFTFGQGTKVDIKRTVAA |
| | | PSVFIFPPSDEQLKSGTASVVCLLNNFYPREAKVQWKVDNALQS |
| | | GNSQESVTEQDSKDSTYSLSSTLTLSKADYEKHKVYACEVTHQG |
| | | LSSPVTKSFNRGEC (SEQ ID NO: 341) |
| | HC | MDMRVPAQLLGLLLLWLRGARCQVQLVQSGAEVKKPGASVKV |
| | | SCKASGYTFTGYYIHWVRQAPGQGLEWMGWINPYSGGTTSAQK |
| | | FQGRVTMTRDTSTSSAYMELSRLRSDDTAVYYCARDAGYLALY |
| | | GTDVWGQGTLVTVSSASTKGPSVFPLAPSSRSTSESTAALGCLVK |
| | | DYFPEPVTVSWNSGALTSGVHTFPAVLQSSGLYSLSSVVTVPSSN |
| | | FGTQTYTCNVDHKPSNTKVDKTVERKCCVECPPCPAPELLGGPS |
| | | VFLFPPKPKDTLMISRTPEVTCVVVDVSHEDPEVKFNWYVDGVE |
| | | VHNAKTKPCEEQYGSTYRCVSVLTVLHQDWLNGKEYKCKVSNK |
| | | ALPAPIEKTISKAKGQPREPQVYTLPPSREEMTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK (SEQ ID NO: 342) |
| 24G6-1 | LC | DIVMTQSPDSLAVSLGERATINCKSSQSVLYSSNNKHFLAWYQQ |
| (SST28347-1) | | KPGQPPKLLIYWASTRESGVPDRFSGSGSGTDFTLTISSLQAEDVA |
| | | VYYCQQYYSTPLTFGGGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| | | (SEQ ID NO: 2768) |
| | HC | EVQLLESGGGLVQPGGSLRLSCAASGFTFSSYAMSWVRQAPGKG |
| | | LEWVSAISGSGGSTYYAESVKGRFTISRDNSKNTLYLQMNSLRAE |
| | | DTAVYYCAKAYTPMAFFDYWGQGTLVTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVD |
| | | VSHEDPEVKFNWYVDGVEVHNAKTKPCEEQYGSTYRCVSVLTV |
| | | LHQDWLNGKEYKCKVSNKALPAPIEKTISKAKGQPREPQVYTLP |
| | | PSREEMTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK (SEQ ID NO: 2769) |
| 24G6-1 | LC | DIVMTQSPDSLAVSLGERATINCKSSQSVLYSSNNKHFLAWYQQ |
| (SST28347-1) | | KPGQPPKLLIYWASTRESGVPDRFSGSGSGTDFTLTISSLQAEDVA |
| | | VYYCQQYYSTPLTFGGGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| | | (SEQ ID NO: 2768) |
| | HC | EVQLLESGGGLVQPGGSLRLSCAASGFTFSSYAMSWVRQAPGKG |
| | | LEWVSAISGSGGSTYYAESVKGRFTISRDNSKNTLYLQMNSLRAE |
| | | DTAVYYCAKAYTPMAFFDYWGQGTLVTVSSASTKGPSVFPLAPS |
| | | SRSTSESTAALGCLVKDYFPEPVTVSWNSGALTSGVHTFPAVLQS |
| | | SGLYSLSSVVTVPSSNFGTQTYTCNVDHKPSNTKVDKTVERKCC |
| | | VECPPCPAPELLGGPSVFLFPPKPKDTLMISRTPEVTCVVVDVSHE |
| | | DPEVKFNWYVDGVEVHNAKTKPCEEQYGSTYRCVSVLTVLHQD |
| | | WLNGKEYKCKVSNKALPAPIEKTISKAKGQPREPQVYTLPPSREE |
| | | MTKNQVSLTCLVKGFYPSDIAVEWESNGQPENNYKTTPPVLDSD |
| | | GSFFLYSKLTVDKSRWQQGNVFSCSVMHEALHNHYTQKSLSLSP |
| | | G (SEQ ID NO: 2770) |
| 6E7-1 | LC | DIQMTQSPSSVSASVGDRVTITCRASQGISSWLAWYQQKPGKAP |
| (SST29857-1) | | KLLIYAASSLQSGVPSRFSGSGSGTDFTLTISSLQPEDFATYFCQQ |
| | | ADAFPRTFGQGTKLEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| | | (SEQ ID NO: 2771) |
| | HC | EVQLVQSGAEVKKPGESLKISCKGSGYSFTSYWIAWVRQMPGKG |
| | | LEWMGIIYPGDADARYSPSFQGQVTISADKSISTAYLQWSSLKAS |
| | | DTAMYFCARQRTFYYDSSDYFDYWGQGTLVTVSSASTKGPSVFP |
| | | LAPSSRSTSESTAALGCLVKDYFPEPVTVSWNSGALTSGVHTFPA |
| | | VLQSSGLYSLSSVVTVPSSNFGTQTYTCNVDHKPSNTKVDKTVE |
| | | RKCCVECPPCPAPELLGGPSVFLFPPKPKDTLMISRTPEVTCVVVD |
| | | VSHEDPEVKFNWYVDGVEVHNAKTKPCEEQYGSTYRCVSVLTV |
| | | LHQDWLNGKEYKCKVSNKALPAPIEKTISKAKGQPREPQVYTLP |
| | | PSREEMTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPG (SEQ ID NO: 2772) |
| 13E7-1 | LC | EIVMTQSPATLSVSPGERATLSCRASQSVSSNLAWFQQKPGQAPR |
| (SST202443-1) | | LLIYGASTRATGIPARFSGSGSGTEFTLTISSLQPEDFAVYYCLQD |
| | | NNFPPTFGQGTKVDIKRTVAAPSVFIFPPSDEQLKSGTASVVCLLN |
| | | NFYPREAKVQWKVDNALQSGNSQESVTEQDSKDSTYSLSSTLTL |
| | | SKADYEKHKVYACEVTHQGLSSPVTKSFNRGEC |
| | | (SEQ ID NO: 2773) |
| | HC | EVQLVQSGAEVKKPGESLKISCKGSGYSFTSYWIGWVRQMPGKG |
| | | LEWMGIIYPGDADARYSPSFQGQVTISADKSISTAYLQWSSLKAS |
| | | DTAMYFCARRRQGIFGDALDFWGQGTLVTVSSASTKGPSVFPLA |
| | | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVV |
| | | DVSHEDPEVKFNWYVDGVEVHNAKTKPCEEQYGSTYRCVSVLT |
| | | VLHQDWLNGKEYKCKVSNKALPAPIEKTISKAKGQPREPQVYTL |
| | | PPSREEMTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPG (SEQ ID NO: 2774) |
| 5E3-1 | LC | DIQMTQSPSSLSASVGDRVTITCRASQGISNYLAWYQQKPGKAPK |
| (SST29825-1) | | SLIYAASSLQSGVPSRFSGSGSGTDFTLTISSLQPEDFATYYCQQY |
| | | STYPFTFGQGTKVDIKRTVAAPSVFIFPPSDEQLKSGTASVVCLLN |
| | | NFYPREAKVQWKVDNALQSGNSQESVTEQDSKDSTYSLSSTLTL |
| | | SKADYEKHKVYACEVTHQGLSSPVTKSFNRGEC |
| | | (SEQ ID NO: 2775) |
| | HC | QVQLVQSGAEVKKPGASVKVSCKASGYTFTGYYIHWVRQAPGQ |
| | | GLEWMGWINPYSGGTTSAQKFQGRVTMTRDTSTSSAYMELSRL |
| | | RSDDTAVYYCARDAGYLALYGTDVWGQGTLVTVSSASTKGPSV |
| | | FPLAPSSRSTSESTAALGCLVKDYFPEPVTVSWNSGALTSGVHTF |
| | | PAVLQSSGLYSLSSVVTVPSSNFGTQTYTCNVDHKPSNTKVDKT |
| | | VERKCCVECPPCPAPELLGGPSVFLFPPKPKDTLMISRTPEVTCVV |
| | | VDVSHEDPEVKFNWYVDGVEVHNAKTKPCEEQYGSTYRCVSVL |
| | | TVLHQDWLNGKEYKCKVSNKALPAPIEKTISKAKGQPREPQVYT |
| | | LPPSREEMTKNQVSLTCLVKGFYPSDIAVEWESNGQPENNYKTTP |
| | | PVLDSDGSFFLYSKLTVDKSRWQQGNVFSCSVMHEALHNHYTQ |
| | | KSLSLSPG (SEQ ID NO: 2776) |
| 13E7 Variant | LC | EIVMTQSPATLSVSPGERATLSCRASQSVSSNLAWFQQKPGQAPR |
| | | LLIYGASTRATGIPARFSGSGSGTEFTLTISSLQPEDFAVYYCLQD |
| | | NNFPPTFGQGTKVDIKRTVAAPSVFIFPPSDEQLKSGTASVVCLLN |
| | | NFYPREAKVQWKVDNALQSGNSQESVTEQDSKDSTYSLSSTLTL |
| | | SKADYEKHKVYACEVTHQGLSSPVTKSFNRGEC |
| | | (SEQ ID NO: 2777) |
| | HC | EVQLVQSGAEVKKPGESLKISCKGSGYSFTSYWIGWVRQMPGKG |
| | | LEWMGIIYPGDADARYSPSFQGQVTISADKSISTAYLQWSSLKAS |
| | | DTAMYFCARRRQGIFGDALDFWGQGTLVTVSSASTKGPSVFPLA |
| | | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVV |
| | | DVSHEDPEVKFNWYVDGVEVHNAKTKPCEEQYGSTYRCVSVLT |
| | | VLHQDWLNGKEYKCKVSNKALPAPIEKTISKAKGQPREPQVYTL |
| | | PPSREEMTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK (SEQ ID NO: 2778) |

In some embodiments, the TREM2 agonist antigen binding proteins of the invention comprise a light chain comprising the sequence of SEQ ID NO:334 and a heavy chain comprising the sequence of SEQ ID NO:335. In some embodiments, the TREM2 agonist antigen binding proteins of the invention comprise a light chain comprising the sequence of SEQ ID NO:334 and a heavy chain comprising the sequence of SEQ ID NO:336. In some embodiments, the TREM2 agonist antigen binding proteins of the invention comprise a light chain comprising the sequence of SEQ ID NO:337 and a heavy chain comprising the sequence of SEQ ID NO:338. In some embodiments, the TREM2 agonist antigen binding proteins of the invention comprise a light chain comprising the sequence of SEQ ID NO:339 and a heavy chain comprising the sequence of SEQ ID NO:340. In some embodiments, the TREM2 agonist antigen binding proteins of the invention comprise a light chain comprising the sequence of SEQ ID NO:341 and a heavy chain comprising the sequence of SEQ ID NO:342. In some embodiments, the TREM2 agonist antigen binding proteins of the invention comprise a light chain comprising the sequence of SEQ ID NO:2768 and a heavy chain comprising the sequence of SEQ ID NO:2769. In some embodiments, the TREM2 agonist antigen binding proteins of the invention comprise a light chain comprising the sequence of SEQ ID NO:2768 and a heavy chain comprising the sequence of SEQ ID NO:2770. In some embodiments, the TREM2 agonist antigen binding proteins of the invention comprise a light chain comprising the sequence of SEQ ID NO:2771 and a heavy chain comprising the sequence of SEQ ID NO:2772. In some embodiments, the TREM2 agonist antigen binding proteins of the invention comprise a light chain comprising the sequence of SEQ ID NO:2773 and a heavy chain comprising the sequence of SEQ ID NO:2774. In some embodiments, the TREM2 agonist antigen binding proteins of the invention comprise a light chain comprising the sequence of SEQ ID NO:2775 and a heavy chain comprising the sequence of SEQ ID NO:2776.

In some embodiments, the present invention provides a method of treating ALSP in a human patient, the method comprising administering to the patient an effective amount of a TREM2 agonist antigen binding protein comprising a light chain comprising the sequence of SEQ ID NO:334 and a heavy chain comprising the sequence of SEQ ID NO:335. In some embodiments, the present invention provides a method of treating ALSP in a human patient, the method comprising administering to the patient an effective amount of a TREM2 agonist antigen binding protein comprising a light chain comprising the sequence of SEQ ID NO:334 and a heavy chain comprising the sequence of SEQ ID NO:336. In some embodiments, the present invention provides a method of treating ALSP in a human patient, the method comprising administering to the patient an effective amount of a TREM2 agonist antigen binding protein comprising a light chain comprising the sequence of SEQ ID NO:337 and a heavy chain comprising the sequence of SEQ ID NO:338. In some embodiments, the present invention provides a method of treating ALSP in a human patient, the method comprising administering to the patient an effective amount of a TREM2 agonist antigen binding protein comprising a light chain comprising the sequence of SEQ ID NO:339 and a heavy chain comprising the sequence of SEQ ID NO:340. In some embodiments, the present invention provides a method of treating ALSP in a human patient, the method comprising administering to the patient an effective amount of a TREM2 agonist antigen binding protein comprising a light chain comprising the sequence of SEQ ID NO:341 and a heavy chain comprising the sequence of SEQ ID NO:342. In some embodiments, the present invention provides a method of treating ALSP in a human patient, the method comprising administering to the patient an effective amount of a TREM2 agonist antigen binding protein comprising a light chain comprising the sequence of SEQ ID NO:2768 and a heavy chain comprising the sequence of SEQ ID NO:2769. In some embodiments, the present invention provides a method of treating ALSP in a human patient, the method comprising administering to the patient an effective amount of a TREM2 agonist antigen binding protein comprising a light chain comprising the sequence of SEQ ID NO:2768 and a heavy chain comprising the sequence of SEQ ID NO:2770. In some embodiments, the present invention provides a method of treating ALSP in a human patient, the method comprising administering to the patient an effective amount of a TREM2 agonist antigen binding protein comprising a light chain comprising the sequence of SEQ ID NO:2771 and a heavy chain comprising the sequence of SEQ ID NO:2772. In some embodiments therefore, the present invention provides a method of treating ALSP in a human patient, the method comprising administering to the patient an effective amount of a TREM2 agonist antigen binding protein comprising a light chain comprising the sequence of SEQ ID NO:2773 and a heavy chain comprising the sequence of SEQ ID NO:2774. In some embodiments, the present invention provides a method of treating ALSP in a human patient, the method comprising administering to the patient an effective amount of a TREM2 agonist antigen binding protein comprising a light chain comprising the sequence of SEQ ID NO:2775 and a heavy chain comprising the sequence of SEQ ID NO:2776. In some embodiments, the present invention provides a method of treating ALSP in a human patient, the method comprising administering to the patient an effective amount of a TREM2 agonist antigen binding protein comprising a light chain comprising the sequence of SEQ ID NO:2777 and a heavy chain comprising the sequence of SEQ ID NO:2778.

In some embodiments, the TREM2 agonist antigen binding proteins of the invention comprise a light chain consisting of or consisting essentially of the amino acid sequence of SEQ ID NO:334, 337, 339 or 341. In some embodiments, the TREM2 agonist antigen binding proteins of the invention comprise a light chain consisting of or consisting essentially of the amino acid sequence of SEQ ID NO:2768, 2771, 2773, or 2775. In some embodiments, the TREM2 agonist antigen binding proteins of the invention comprise a heavy chain consisting of or consisting essentially of the amino acid sequence of SEQ ID NO:335, 336, 338, 340, or 342. In some embodiments, the TREM2 agonist antigen binding proteins of the invention comprise a heavy chain consisting of or consisting essentially of the amino acid sequence of SEQ ID NO:2769, 2770, 2772, 2774, or 2776. In a specific embodiment, the TREM2 agonist antigen binding proteins of the invention comprise a light chain and a heavy chain, wherein:
(a) the light chain consisting of or consisting essentially of the amino acid sequence of SEQ ID NO:334 and the heavy chain consisting of or consisting essentially of the amino acid sequence of SEQ ID NO:335;
(b) the light chain consisting of or consisting essentially of the amino acid sequence of SEQ ID NO:334 and the heavy chain consisting of or consisting essentially of the amino acid sequence of SEQ ID NO:336;
(c) the light chain consisting of or consisting essentially of the amino acid sequence of SEQ ID NO:337 and the heavy chain consisting of or consisting essentially of the amino acid sequence of SEQ ID NO:338;
(d) the light chain consisting of or consisting of essentially of the amino acid sequence of SEQ ID NO:339 and the heavy chain consisting of or consisting essentially of the amino acid sequence of SEQ ID NO:340; or
(e) the light chain consisting of or consisting essentially of the amino acid sequence of SEQ ID NO:341 and the heavy chain consisting of or consisting essentially of the amino acid sequence of SEQ ID NO:342.

In a specific embodiment, the TREM2 agonist antigen binding proteins of the invention comprise a light chain and a heavy chain, wherein:
(a) the light chain consisting of or consisting essentially of the amino acid sequence of SEQ ID NO:2768 and the heavy chain consisting of or consisting essentially of the amino acid sequence of SEQ ID NO:2769;
(b) the light chain consisting of or consisting essentially of the amino acid sequence of SEQ ID NO:2768 and the heavy chain consisting of or consisting essentially of the amino acid sequence of SEQ ID NO:2770;
(c) the light chain consisting of or consisting essentially of the amino acid sequence of SEQ ID NO:2771 and the heavy chain consisting of or consisting essentially of the amino acid sequence of SEQ ID NO:2772;
(d) the light chain consisting of or consisting of essentially of the amino acid sequence of SEQ ID NO:2773 and the heavy chain consisting of or consisting essentially of the amino acid sequence of SEQ ID NO:2774;
(e) the light chain consisting of or consisting essentially of the amino acid sequence of SEQ ID NO:2775 and the heavy chain consisting of or consisting essentially of the amino acid sequence of SEQ ID NO:2776; or
(f) the light chain consisting of or consisting essentially of the amino acid sequence of SEQ ID NO:2777 and the heavy chain consisting of or consisting essentially of the amino acid sequence of SEQ ID NO:2778.

Unless indicated otherwise by reference to a specific sequence and in related discussions, the numbering of the amino acid residues in an immunoglobulin heavy chain or light chain is according to Kabat-EU numbering as described in Kabat et al., Sequences of Proteins of Immunological Interest, 5th Ed., US Department of Health and Human Services, NIH publication No. 91-3242, pp 662,680,689 (1991) and Edelman et al., Proc. Natl. Acad. USA, Vol. 63: 78-85 (1969). The Kabat numbering scheme is typically used when referring to the position of an amino acid within the variable regions, whereas the EU numbering scheme is generally used when referring to the position of an amino acid with an immunoglobulin constant region.

In some embodiments, the TREM2 antigen binding protein comprise an antibody that competes with an antibody comprising CDRL1, CDRL2, CDRL3 or light chain variable region disclosed in TABLES A1, A10, A12, and A14, and a heavy chain variable region disclosed in TABLES A2, A11, A13, and A14. In some embodiments, a suitable assay for detecting competitive binding employs kinetic sensors used with Octet® systems (Pall ForteBio), which measures binding interactions using bio-layer interferometry methodology. One group of antibodies, antibodies 10E3, 13E7, 24F4, 4C5, 4G10, 32E3, and 6E7, competed with each other for binding to human TREM2, indicating that they share the same or similar epitope on human TREM2. Antibodies 16B8, 26A10, 26C10, 26F2, 33B12, and 5E3 compete with each other for TREM2 binding, but does not compete with antibodies in the first group or antibodies 24A10, 24G6, or 25F12, indicating that this second group of antibodies bind to a distinct epitope on human TREM2. Antibodies 24A10 and 24G6 share a similar epitope on human TREM2 as these two antibodies compete with each other for human TREM2 binding, but did not compete with any other antibody. Antibody 25F12 did not compete with any of the other tested antibodies for human TREM2 binding, indicating that this antibody binds to yet another epitope.

In some embodiments, a TREM2 agonist antigen binding protein competes with a reference antibody for binding to human TREM2, wherein the reference antibody comprises a light chain variable region comprising a sequence selected from SEQ ID NOS:46-63 and a heavy chain variable region comprising a sequence selected from SEQ ID NOS: 110-126. In other embodiments, a TREM2 agonist antigen binding protein of the invention competes with a reference antibody for binding to human TREM2, wherein the reference antibody comprises a light chain variable region comprising a sequence selected from SEQ ID NOS: 153-162 and a heavy chain variable region comprising a sequence selected from SEQ ID NOS: 180-190. In still other embodiments, a TREM2 agonist antigen binding protein of the invention competes with a reference antibody for binding to human TREM2, wherein the reference antibody comprises a light chain variable region comprising a sequence selected from SEQ ID NOS:61 and 295-300 and a heavy chain variable region comprising a sequence selected from SEQ ID NOS: 124 and 307-312. In certain embodiments, a TREM2 agonist antigen binding protein of the invention competes for binding to human TREM2 with one or more of the anti-TREM2 antibodies described herein, including 12G10, 26A10, 26C10, 26F2, 33B12, 24C12, 24G6, 24A10, 10E3, 13E7, 14C12, 25F12, 32E3, 24F4, 16B8, 4C5, 6E7, 5E3, 4G10, V3, V9, V10, V23, V24, V27, V30, V33, V40, V44, V48, V49, V52, V57, V60, V68, V70, V73, V76, V83, V84, and V90.

In some embodiments, the TREM2 agonist antigen binding protein competes with a reference antibody for binding to human TREM2, wherein the reference antibody comprises a light chain variable region comprising the sequence of SEQ ID NO:61 and a heavy chain variable region comprising the sequence of SEQ ID NO: 124. In such embodiments, antigen binding proteins that compete with this reference antibody for binding to human TREM2 would bind the same or similar epitope as antibody 6E7 or any of the other antibodies 10E3, 13E7, 24F4, 4C5, 4G10, and 32E3.

In some embodiments, the TREM2 agonist antigen binding protein competes with a reference antibody for binding to human TREM2, wherein the reference antibody comprises a light chain variable region comprising the sequence of SEQ ID NO:62 and a heavy chain variable region comprising the sequence of SEQ ID NO: 125. In such embodiments, antigen binding proteins that compete with this reference antibody for binding to human TREM2 would bind the same or similar epitope as antibody 5E3 or any of the other antibodies 16B8, 26A10, 26C10, 26F2, and 33B12.

In some embodiments, the TREM2 agonist antigen binding protein competes with a reference antibody for binding to human TREM2, wherein the reference antibody comprises a light chain variable region comprising the sequence of SEQ ID NO:52 and a heavy chain variable region comprising the sequence of SEQ ID NO: 115. In such embodiments, antigen binding proteins that compete with this reference antibody for binding to human TREM2 would bind the same or similar epitope as antibody 24G6 or antibody 24A10.

In some embodiments, the TREM2 agonist antigen binding protein competes with a reference antibody for binding to human TREM2, wherein the reference antibody comprises a light chain variable region comprising the sequence of SEQ ID NO:56 and a heavy chain variable region comprising the sequence of SEQ ID NO: 119. In such embodiments, antigen binding proteins that compete with this reference antibody for binding to human TREM2 would bind the same or similar epitope as antibody 25F12.

In some embodiments, isolated nucleic acids encoding the anti-TREM2 binding domain of the antigen binding proteins of the invention can be used to synthesize the antigen binding protein or used to generate variants. In some embodiments, the polynucleotide may comprise a nucleotide sequence that is at least 80% identical, at least 90% identical, at least 95% identical, or at least 98% identical to any of the nucleotide sequences listed in TABLE A15..

TABLE A16
Exemplary Anti-TREM2 Antibody Variable Region Nucleic Acid Sequences

| | | |
|---|---|---|
| | VL or VH | |
| | Group | |
| Ab ID. | Designation | Nucleic Acid Sequence |

Light chain variable regions

| | | |
|---|---|---|
| 12G10 | LV-01 | CAGGCTGTGCCGACTCAGCCGTCTTCCCTCTCTGCATCTCCTGGAGT |
| | | ATTAGCCAGTCTCACCTGCACCTTACGCAGTGGCATCAATGTTGGTA |
| | | CCTACAGGATATACTGGTACCAGCAGAAGCCAGGGAGTCCTCCCCA |
| | | GTATCTCCTGAGGTACAAATCAGACTCAGATAAGCAGCAGGGCTCT |
| | | GGAGTCCCCAGCCGCTTCTCTGGATCCAAGGATGCTTCGGCCAATGC |
| | | AGGGATTTTACTCATCTCTGGGCTCCAGTCTGAGGATGAGGCTGACT |
| | | ATTACTGTATGATTTGGTACAGCAGTGCTGTGGTATTCGGCGGAGGG |
| | | ACCAAACTGACCGTCCTA (SEQ ID NO: 208) |
| 26A10 | LV-02 | TCCTATGAGCTGACTCAGCCACCCTCAGTGTCCGTGTCCCCAGGACA |
| | | GACAGCCAGCATCACCTGCTCTGGAGATAAATTGGGAGATAAGTAT |
| | | GTTTGCTGGTATCAGCAGAAGCCAGGCCAGTCCCCTGTGCTGGTCAT |
| | | CTATCAAGATAGCAAGCGGCCCTCAGGGATCCCTGAGCGATTCTCTG |
| | | GCTCCAACTCTGGGAACACAGCCACTCTGACCATCAGCGGGACCCA |
| | | GGCTATGGATGAGGCTGACTATTACTGTCAGGCGTGGGACAGTAAC |
| | | ACTGTGGTATTCGGCGGAGGGACCAAGCTGACCGTCCTA |
| | | (SEQ ID NO: 209) |
| 26C10 | LV-03 | TCCTTTGAGCTGACTCAGCCACCCTCAGTGTCCGTGTCCCCAGGACA |
| | | GACAGCCAGCATCACCTGCTCTGGAGATAAATTGGGGGATAAGTAT |
| | | GTTTGCTGGTATCAGCAGAAGCCAGGCCAGTCCCCTATGTTGGTCAT |
| | | CTATCAAGATACCAAGCGGCCCTCAGGGATCCCTGAACGATTCTCTG |
| | | GCTCCAACTCTGGGAACACAGCCACTCTGACCATCAGCGGGACCCA |
| | | GGCTATGGATGAGGCTGACTATTACTGTCAGGCGTGGGACAGCAGC |
| | | ACTGTGGTCTTCGGCGGAGGGACCAAGCTGACCGTCCTA |
| | | (SEQ ID NO: 210) |
| 26F2 | LV-04 | TCCTATGAGCTGACTCAGCCACCCTCAGTGTCCGTGTCCCCAGGACA |
| | | GACAGCCAGCATCACCTGCTCTGGAGATAAATTGGGGGATAAGTAT |
| | | GTTTGCTGGTATCAGCAGAAGCCAGGCCAGTCCCCTGTGTTGGTCAT |
| | | CTTTCAAGATAGCAAGCGGCCCTCAGGGATCCCTGAGCGATTCTCTG |
| | | GCTCCAACTCTGGGAACACAGCCACTCTGACCATCAGCGGGACCCA |
| | | GGCTATGGATGAGGCTGACTATTACTGTCAGGCGTGGGACAGCAGC |
| | | ACTGTGGTATTCGGCGGAGGGACCAAGCTGACCGTCCTA |
| | | (SEQ ID NO: 211) |
| 33B12 | LV-05 | TCCTATGAGCTGACTCAGCCACCCTCAGTGTCCGTGTCCCCAGGACA |
| | | GACAGCCAGCATCACCTGCTCTGGAGATAAATTGGGGGATAAGTAT |
| | | GTTTGCTGGTATCAGCAGAAGCCAGGCCAGTCCCCTGTGTTGGTCAT |
| | | CTATCAAGATAGCAAGCGGCCCTCAGGGATCCCTGAGCGATTCTCTG |
| | | GCTCCAACTCTGGGAACACAGCCACTCTGACCATCAGCGGGACCCA |
| | | GGCTATGGATGAGGCTGACTATTACTGTCAGGCGTGGGACAGTAGC |
| | | ACTGTGGTATTCGGCGGAGGGACCAAGCTGACCGTCCTA |
| | | (SEQ ID NO: 212) |
| 24C12 | LV-06 | GGCATCGTGATGACCCAGTCTCCAGACTCCCTGGCTGTGTCTCTGGG |
| | | CGAGAGGGCCACCATCAACTGCAAGTCCAGCCGGAGTGTTTTGTAC |
| | | AGCTCCAACAATAAGAACTACTTAGCTTGGTACCAGCAGAAACCAG |
| | | GACAGCCTCCTAAGGTGCTCATTTACTGGGCATCTACCCGGGAATCC |
| | | GGGGTCCCTGACCGATTCAGTGGCAGCGGGTCTGGGACAGATTTCA |
| | | CTCTCACCATCAGCAGCCTGCAGGCTGAAGATGTGGCAGTTTATAAC |
| | | TGTCAGCAATATTATATTACTCCGATCACCTTCGGCCAAGGGACACG |
| | | ACTGGAGATTAAA (SEQ ID NO: 213) |
| 24G6 | LV-07 | GACATCGTGATGACCCAGTCTCCAGACTCCCTGGCTGTGTCTCTGGG |
| | | CGAGAGGGCCACCATCAACTGCAAGTCCAGCCAGAGTGTTTTATAC |
| | | AGCTCCAACAATAAGCACTTCTTAGCTTGGTACCAGCAGAAACCAG |
| | | GACAGCCTCCTAAGCTGCTCATTTACTGGGCATCTACCCGGGAGTCC |
| | | GGGGTCCCTGACCGATTCAGTGGCAGCGGGTCTGGGACAGATTTCA |
| | | CTCTCACCATCAGCAGCCTGCAGGCTGAAGATGTGGCATTTTATTAC |
| | | TGTCAGCAATATTATAGTACTCCGCTCACTTTCGGCGGAGGGACCAA |
| | | GGTGGAGATCAAA (SEQ ID NO: 214) |
| 24A10 | LV-08 | GACATCGTGATGACCCAGTCTCCAGACTCCCTGGCTGTGTCTCTGGG |
| | | CGAGAGGGCCACCATCACCTGCAAGTCCAGCCACAATGTTTTATACA |
| | | GCTCCAACAATAAGAACTACTTAGCTTGGTATCAGCAGAAACCAGG |
| | | ACAGCCTCCTAAACTGCTCATTTACTGGGCATCTACCCGGGAATCCG |
| | | GGGTCCCTGACCGATTCAGTGGCAGCGGGTCTGGGACAGATTTCACT |
| | | CTCACCATCAGCAGCCTGCAGGCTGAAGATGTGGCAGTTTATTACTG |
| | | TCACCAATATTATAGTACTCCGTGCAGTTTTGGCCAGGGGACCAAGC |
| | | TGGAGATCAAA (SEQ ID NO: 215) |
| 10E3 | LV-09 | GAAATAGTGATGACGCAGTCTCCAGCCACCCTGTCTGTGTCTCCAGG |
| | | GGAAAGAGCCACCCTCTCCTGCAGGGCCAGTCAGAGTGTTAGCAGC |
| | | AACTTAGCCTGGTTCCAGCAGAAACCTGGCCAGGCTCCCAGGCTCCT |
| | | CATCTATGGTGCTTCCACCAGGGCCACTGGTATTCCAGCCAGGTTCA |
| | | GTGTCAGTGGGTCTGGGACAGAGTTCACTCTCACCATCAGCAGCCTG |
| | | CAGTCTGAAGATTTTGCATTTTATTACTGTCTGCAGGATAATAATTG |
| | | GCCTCCCACTTTCGGCCCTGGGACCAAAGTGGATATCAAA |
| | | (SEQ ID NO: 216) |
| 13E7 | LV-10 | GAAATAGTGATGACGCAGTCTCCAGCCACCCTGTCTGTGTCTCCAGG |
| | | GGAAAGAGCCACCCTCTCCTGCAGGGCCAGTCAGAGTGTTAGCAGC |
| | | AACTTAGCCTGGTTCCAGCAGAAACCTGGCCAGGCTCCCAGGCTCCT |
| | | CATCTATGGTGCTTCCACCAGGGCCACTGGTATTCCAGCCAGGTTCA |
| | | GTGTCAGTGGGTCTGGGACAGAGTTCACTCTCACCATCAGCAGCCTG |
| | | CAGTCTGAAGATTTTGCAGTTTATTACTGTCTGCAGGATAATAATTG |
| | | GCCTCCCACTTTCGGCCCTGGGACCAAAGTGGATATCAAA |
| | | (SEQ ID NO: 217) |
| 25F12 | LV-11 | GAAAAAGTGATGACGCAGTCTCCAGCCACCCTGTCTGTGTCTCCAGG |
| | | GGAAAGAGCCACCCTCTCCTGCAGGGCCAGTCAGAGTGTTAACAAC |
| | | AACTTAGCCTGGTACCAGCAGAAACCTGGCCAGGCTCCCAGGCTCCT |
| | | CATCTATGGTGCATCCACCAGGGCCACTGGTATCCCAGCCAGGTTCA |
| | | GTGGCAGTGGGTCTGGGACAGAGTTCACTCTCACCATCAGCAGCCTG |
| | | CAGTCTGAAGATTTTGCAGTTTATTACTGTCAGCAGTATAATAACTG |
| | | GCCTCGGACGTTCGGCCAAGGGACCAAGGTGGAAATCAAA |
| | | (SEQ ID NO: 218) |
| 32E3 | LV-12 | GAATTTGTGTTGACGCAGTCTCCAGGCACCCTGTCTTTGTCTCCGGG |
| | | GGAAAGAGCCACCCTCTCCTGCAGGGCCAGTCAGATTATTAGCAGC |
| | | AACTACTTAGCCTGGTACCAGCAGAAACCTGGCCAGGCTCCCAGGC |
| | | TCCTCATCTATAGTGCATCCAGCAGGGCCACTGGCATCCCAGACAGG |
| | | TTCAGTGGCAGTGGGTCTGGGACAGACTTCACTCTCACCATCAGCAG |
| | | ACTGGAGCCTGAAGATTTTGCAGTGTATTACTGTCAGCAGTTTGATA |
| | | GCTCACCGATCACCTTCGGCCGAGGGACACGACTGGACATTAAA |
| | | (SEQ ID NO: 219) |
| 24F4 | LV-13 | GAAATTGTGTTGACGCAGTCTCCAGGCACCCTGTCTTTGTCTCCAGG |
| | | GGAAAGAGCCACCCTCTCCTGCAGGGCCAGTCAGAGTGTTAGCAGC |
| | | AGCTACTTAGCCTGGTACCAGCAGAAACCTGGCCAGGCTCCCAGGC |
| | | TCCTCATCTATGGTGCATCCAGCAGGGCCACTGGCATCCCAGACAGG |
| | | TTCAGTGGCAGTGGGTCTGGGACAGACTTCACTCTCACCATCAGCAG |
| | | ACTGGAGCCTGAAGATTTTGCACTGTATTACTGTCAGCAGTATGATA |
| | | CCTCACCATTCACTTTCGGCCCTGGGACCAAAGTGGATATCAAA |
| | | (SEQ ID NO: 220) |
| 16B8 | LV-14 | GACATCCAGATGACCCAGTCTCCATCTTCCGTGTCTGCATCTGTAGG |
| | | AGACAGAGTCACCGTCACTTGTCGGGCGAGTCAGGATATTAACAGC |
| | | TGGTTAGCCTGGTATCAGCAGAAACCAGGGAAAGCCCCTAAGCTCC |
| | | TGATCTATGCTGCATCCTCTTTGCAAACTGGGGTCCCTTCAAGGTTC |
| | | AGCGGCAGTGGATCTGGGACAGATTTCACTCTCACCATCAGCAGCCT |
| | | GCAGCCTGAAGATTTTGCAACTTACTCTTGTCAACAGTCTAACAGTT |
| | | TCCCGATCACCTTCGGCCAAGGGACACGACTGGAGATTAAA |
| | | (SEQ ID NO: 221) |
| 4C5 | LV-15 | GACATCCAGATGACCCAGTCTCCATCTTCCGTGTCTGCATCTGTAGG |
| | | AGACAGAGTCACCATCACTTGTCGGGCGAGTCAGGGTATTAGCAAC |
| | | TGGTTAGCCTGGTATCAGCAGAAACCAGGGAAAGCCCCTAAGCTCC |
| | | TGATCTATGCTGCATCCAGTTTGCAAGTTGGGGTCCCATTAAGGTTC |
| | | AGCGGCAGTGGATCTGGGACAGATTTCACTCTCACCATCAGCAGCCT |
| | | GCAGCCTGAAGATTTTGCAACTTACTATTGTCAACAGGCTGACAGTT |
| | | TCCCTCGCAATTTTGGCCAGGGGACCAAGCTGGAGATCAAA |
| | | (SEQ ID NO: 222) |
| 6E7 | LV-16 | GACATCCAGATGACCCAGTCTCCATCTTCCGTGTCTGCATCTGTAGG |
| V9 | | AGACAGAGTCACCATCACTTGTCGGGCGAGTCAGGGTATTAGCAGC |
| V30 | | TGGTTAGCCTGGTATCAGCAGAAACCAGGGAAAGCCCCTAAGCTCC |
| V33 | | TGATCTATGCTGCATCCAGTTTGCAAAATGGGGTCCCATCAAGGTTC |
| V44 | | AGCGGCAGTGGATCTGGGACAGATTTCACTCTCACCATCAGCAGCCT |
| V68 | | GCAGCCTGAAGATTTTGCAACTTACTTTTGTCAACAGGCTGACAGTT |
| | | TCCCTCGCACTTTTGGCCAGGGGACCAAGCTGGAGATCAAA |
| | | (SEQ ID NO: 223) |
| 5E3 | LV-17 | GACATCCAGATGACCCAGTCTCCATCCTCACTGTCTGCATCTGTAGG |
| | | AGACAGAGTCACCATCACTTGTCGGGCGAGTCAGGGCATTAGCAAT |
| | | TATTTAGCCTGGTTTCAGCAGAAACCAGGGAAAGCCCCTAAATCCCT |
| | | GATCTATGCTGCATCCAGTTTGCAAAGTGGGGTCCCATCAAAGTTCA |
| | | GCGGCAGTGGATCTGGGACAGATTTCACTCTCACCATCAGCAGCCTG |
| | | CAGCCTGAAGATTTTGCAACTTATTACTGCCAACAGTATAGTACTTA |
| | | CCCATTCACTTTCGGCCCTGGGACCAAAGTGGATATCAAA |
| | | (SEQ ID NO: 224) |
| 4G10 | LV-18 | GACATCCAGATGACCCAGTCTCCATCCTCCCTGTCTGCATCTGTAGG |
| | | AGACAGAGTCACCATCACTTGCCGGGCAAGTCAGGGCATAAGAAAT |
| | | GATTTAGGCTGGTATCAGCAGAAACCAGGGAATGCCCCTAAGCGCC |
| | | TGATCTATGCTGCATCCAGTTTGCCAAGTGGGGTCCCATCAAGGTTC |
| | | AGCGGCAGTGGATCTGGGCCAGAATTCACTCTCACAATCAGCAGTCT |
| | | GCAGCCTGAAGATTTTGCAACTTATTACTGTCTACAGCATAATAGTT |
| | | ACCCGTGGACGTTCGGCCAAGGGACCAAGGTGGAAATCACA |
| | | (SEQ ID NO: 225) |
| V3 | LV-101 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATGCTGCATCCAGTAGGCAAAATGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGACAGGT |
| | | TCCCTCGCACTTTTGGCCAGGGGACCAAGCTGGAGATCAAA |
| | | (SEQ ID NO: 226) |
| V24 | LV-102 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATGCTGCATCCAGTTTGCAAAAGGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGACAGTT |
| | | TCCCTCATACTTTTGGCCAGGGGACCAAGCTGGAGATCAAA |
| | | (SEQ ID NO: 227) |
| V27 | LV-103 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATGCTGCATCCAGTTTGCAACGTGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGACAGTT |
| | | TCCCTCGCACTTTTGGCCAGGGGACCAAGCTGGAGATCAAA |
| | | (SEQ ID NO: 228) |
| V40 | LV-104 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATGCTGCATCCAGTTTGCAACTTGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGACCGTT |
| | | TCCCTCGCACTTTTGGCCAGGGGACCAAGCTGGAGATCAAA |
| | | (SEQ ID NO: 229) |
| V48 | LV-105 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATGCTGCATCCAGTTTGCAAACGGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGACAGTT |
| | | TGCCTCGCACTTTTGGCCAGGGGACCAAGCTGGAGATCAAA |
| | | (SEQ ID NO: 230) |
| V49 | LV-106 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATGCTGCATCCAGTCGGCAAAATGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGACAGTT |
| | | ATCCTCGCACTTTTGGCCAGGGGACCAAGCTGGAGATCAAA |
| | | (SEQ ID NO: 231) |
| V52 | LV-107 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATGCTGCATCCAGTTTGCAAAGGGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGACCGTT |
| | | TCCCTCGCACTTTTGGCCAGGGGACCAAGCTGGAGATCAAA |
| | | (SEQ ID NO: 232) |
| V60 | LV-108 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATGCTGCATCCAGTTTGCAAAGGGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTGGGCAGGCTGACAGTT |
| | | TCCCTCGCACTTTTGGCCAGGGGACCAAGCTGGAGATCAAA |
| | | (SEQ ID NO: 233) |
| V73 | LV-106 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATGCTGCATCCAGTCGTCAAAATGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGACAGTT |
| | | ATCCTCGCACTTTTGGCCAGGGGACCAAGCTGGAGATCAAA |
| | | (SEQ ID NO: 234) |
| V76 | LV-109 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATGCTGCATCCAGTTTGCAAAAGGGGGTCCCATCAAGGTTC |
| | | AGCGGCAGTGGATCTGGGAGAGATTTCACTCTCACCATCAGCAGCCT |
| | | GCAGCCTGAAGATTTTGCAACTTACTTTTGTCAACAGGCTGACAGTT |
| | | TCCCTCGCACTTTTGGCCAGGGGACCAAGCTGGAGATCAAA |
| | | (SEQ ID NO: 235) |
| V84 | LV-110 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATGGTGCATCCAGTTTGCAAAATGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGACAGTT |
| | | TCCCGCGCACTTTTGGCCAGGGGACCAAGCTGGAGATCAAA |
| | | (SEQ ID NO: 236) |
| V10 | LV-201 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATTCTGCATCCAGTTTGCAAAATGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGACAGTT |
| | | TCCCTCGCACTTTTGGCCAGGGGACCAAGCTGGAGATCAAA |
| | | (SEQ ID NO: 313) |
| V23 | LV-202 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATGCTGCATCCAGTTTGCAAAATGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGACAGTT |
| | | TCCCTCTTACTTTTGGCCAGGGGACCAAGCTGGAGATCAAA |
| | | (SEQ ID NO: 314) |
| V57 | LV-203 | GACATCCAGATGACCCAGTCTCCATCTTCCGTGTCTGCATCTGTAGG |
| | | AGACAGAGTCACCATCACTTGTGCGGCGAGTCAGGGTATTAGCAGC |
| | | TGGTTAGCCTGGTATCAGCAGAAACCAGGGAAAGCCCCTAAGCTCC |
| | | TGATCTATGCTGCATCCAGTTTGCAAAATGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGACAGTT |
| | | TCCCTCGCACTTTTGGCCAGGGGACCAAGCTGGAGATCAAA |
| | | (SEQ ID NO: 315) |
| V70 | LV-204 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATGCTGCAGGGAGTTTGCAAAATGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGACAGTT |
| | | TCCCTCGCACTTTTGGCCAGGGGACCAAGCTGGAGATCAAA |
| | | (SEQ ID NO: 316) |
| V83 | LV-205 | GACATCCAGATGACCCAGTCTCCATCTTCCGTGTCTGCATCTGTAGG |
| | | AGACAGAGTCACCATCACTTGTCGGGCGAGTCAGGGTATTAGCAGC |
| | | TGGTTAGCCTGGTATCAGCAGAAACCAGGGAAAGCCCCTAAGCTCC |
| | | TGATCTATGCTGCATCCAGTTTGCAAAATGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGTGAGTT |
| | | TCCCTCGCACTTTTGGCCAGGGGACCAAGCTGGAGATCAAA |
| | | (SEQ ID NO: 317) |
| V90 | LV-206 | GACATCCAGATGACCCAGTCTCCATCTTCCGTGTCTGCATCTGTAGG |
| | | AGACAGAGTCACCATCACTTGTCGGGCGAGTCAGGGTATTAGCAGA |
| | | TGGTTAGCCTGGTATCAGCAGAAACCAGGGAAAGCCCCTAAGCTCC |
| | | TGATCTATGCTGCATCCAGTTTGCAAAATGGGGTCCCATCAAGGTTC |
| | | AGCGGCAGTGGATCTGGGACAGATTTCACTCTCACCATCAGCAGCCT |
| | | GCAGCCTGAAGATTTTGCAACTTACTTTTGTCAACAGGCTGACAGTT |
| | | TCCCTCGCACTTTTGGCCAGGGGACCAAGCTGGAGATCAAA |
| | | (SEQ ID NO: 318) |

Heavy chain variable regions

| | | |
|---|---|---|
| 12G10 | HV-01 | GAGGTGCAGCTGTTGGAGTCTGGGGGAGGCTTGGTACAGCCTGGGG |
| 24C12 | | GGTCCCTGAGACTCTCCTGTGCAGCCTCTGGATTCACCTTTAGCAGC |
| | | TATGCCATGAGCTGGGTCCGCCAGGCTCCAGGGAAGGGGCTGGAGT |
| | | GGGTCTCAGCTATTGGTGGTGGTGGTGTTAGCACATACTGCGCAGAC |
| | | TCCGTGAAGGGCCGGTTCACCATCTCCAGAGACAATTCCAAGAATA |
| | | CGCTGTATCTGCAAATGAACAGCCTGAGAGCCGAGGACACGGCCGT |
| | | ATATTACTGTGCGAAATTTTATATAGCAGTGGCTGGTTCTCACTTTG |
| | | ACTACTGGGGCCAGGGAACCCTGGTCACCGTCTCCTCA |
| | | (SEQ ID NO: 237) |
| 26A10 | HV-02 | GAGGTGCAACTGGTGGAGTCTGGGGGAGCCTTGGTACAGCGGGGGG |
| | | GGTCCCTGAGACTCTCCTGTGCAGCCTCTAGATTCACCTTCAGTAGC |
| | | TTTGGCATGAGCTGGGTCCGCCAGGCTCCAGGGAAGGGGCTGGAGT |
| | | GGGTTTCATACATTAGTAGTAGTAGTTTTACCATATATTACGCAGAC |
| | | TCTGTGAAGGGCCGATTCACCATCTCCAGAGACAATGCCAAGAATTC |
| | | ATTCTATCTGCAAATGAACAGCCTGAGAGACGAGGACACGGCTGTG |
| | | TATTACTGTGCGAGAGAGGGGGGTCTTACTATGGTTCGGGGAGTCTC |
| | | TTCCTACGGTTTGGACGTCTGGGGCCAAGGGACCACGGTCACCGTCT |
| | | CCTCA (SEQ ID NO: 238) |
| 26C10 | HV-03 | GAGGTGCAACTGGTGGAGTCTGGGGGAGCCTTGGTACAGCCTGGGG |
| | | GGTCCCTGAGACTCTCCTGTGCAGCCTCTGGATTCACCTTCAGTAGC |
| | | TTTGGCATGAGCTGGGTCCGCCAGGCTCCAGGGAAGGGGCTGGAGT |
| | | GGGTTTCATACATTAGTAGTAGTAGTTTTACCATATACTACGCAGAC |
| | | TCTGTGAAGGGCCGATTCACCATCTCCAGAGACAATGCCAAGAATTC |
| | | GTTCTATCTGCAAATGAACAGCCTGAGAGACGAGGACACGGCTGTG |
| | | TATTTCTGTGTGAGAGAGGGGGGTATAACTATGGTTCGGGGAGTCTC |
| | | TTCCTACGGTATGGACGTCTGGGGCCAAGGGACCACGGTCACCGTCT |
| | | CCTCA (SEQ ID NO: 239) |
| 26F2 | HV-04 | GAGGTGCAACTGGTGGAGTCTGGGGGAGCCTTGGTACAGCCTGGGG |
| | | GGTCCCTGAGACTCTCCTGTGCAGCCTCTGGATTCACCTTCAGTAGC |
| | | TTTGGCATGAGCTGGGTCCGCCAGGCTCCAGGGAAGGGGCTGGAGT |
| | | GGATTTCATACATTAGTAGTAGTAGTTTTACCATATACTACGCAGAC |
| | | TCTGTGAAGGGCCGATTCACCATCTCCAGAGACAATGCCAAGAATTC |
| | | ATTCTATCTGCAAATGAACAGCCTGAGAGACGAGGACACGGCTGTG |
| | | TATTTCTGTGCGAGAGAGGGGGGTATTACTATGGTTCGGGGAGTCTC |
| | | TTCCTACGGTATGGACGTCTGGGGCCAAGGGACCACGGTCACCGTCT |
| | | CCTCA (SEQ ID NO: 240) |
| 33B12 | HV-05 | GAGGTGCAACTGGTGGAGTCTGGGGGAGCCTTGGTACAGCCTGGGG |
| | | GGTCCCTGAGACTCTCCTGTGCAGCCTCTGGATTCACCTTCAGTAGC |
| | | TTTGGCATGAGCTGGGTCCGCCAGGCTCCAGGGAAGGGCCTGGAGT |
| | | GGGTTTCATACATTAGTAAAAGTAGTTTTACCATATACTACGCAGAC |
| | | TCTGTGAAGGGCCGATTCACCATCTCCAGAGACAATGCCAAGAATTC |
| | | ATTCTATCTGCAAATGAACAGCCTGAGAGACGAGGACACGGCTGTG |
| | | TATTACTGTGCGAGAGAGGGGGGTCTTACTATGGTTCGGGGAGTCTC |
| | | TTCCTACGGTTTGGACGTCTGGGGCCAAGGGACCACGGTCACCGTCT |
| | | CCTCA (SEQ ID NO: 241) |
| 24G6 | HV-06 | GAGGTGCAGCTGTTGGAGTCTGGGGGAGGCTTGGTACAGCCTGGGG |
| | | GGTCCCTGAGACTCTCCTGTGCAGCCTCTGGATTCACCTTTAGCAGC |
| | | TATGCCATGAGCTGGGTCCGCCAGGCTCCAGGGAAGGGACTGGAGT |
| | | GGGTCTCAGCTATTAGTGGTAGTGGTGGTAGCACATACTACGCAGAC |
| | | TCCGTGAAGGGCCGGTTCACCATCTCCAGAGACAATTCCAAGAACA |
| | | CGCTGTATCTGCAAATGAACAGCCTGAGAGCCGAGGACACGGCCGT |
| | | ATATTACTGTGCGAAGGCGTATACACCTATGGCATTCTTTGACTACT |
| | | GGGGCCAGGGAACCCTGGTCACCGTCTCCTCA (SEQ ID NO: 242) |
| 24A10 | HV-07 | GAGGTGCAGGTGTTGGAGTCTGGGGGAGGCTTGGTACAGCCTGGGG |
| | | GGTCCCTGAGACTCTCCTGTGCAGCCTCTGGATTCACCTTTAGCAAC |
| | | TATGCCATGAGCTGGGTCCGCCAGGCTCCAGGGAAGGGGCTGGAGT |
| | | GGGTCTCAGCTATTAGTGGTAGTGGTGGTAGCACATACTACGCAGAC |
| | | TCCGTGAAGGGCCGGTTCACCATCTCCAGAGACAATTCCAAGAACA |
| | | CGCTGTATCTGCAAATGAACAGCCTGAGAGCCGAGGACACGGCCGT |
| | | ATATTACTGTGCGAAAGGAGGGTGGGAGCTATTTTACTGGGGCCAG |
| | | GGAACCCTGGTCACCGTCTCCTCA (SEQ ID NO: 243) |
| 10E3 | HV-08 | GAGGTGCAGCTGGTGCAGTCTGGAGCAGAGGTGAAAAAGCCCGGGG |
| | | AGTCTCTGATGATCTCCTGTAAGGGTTCTGGATACAGCTTTACCAAC |
| | | TACTGGATCGGCTGGGTGCGCCAGATGCCCGGGAAAGGCCTGGAGT |
| | | GGATGGGGATCATCTATCCTGGAGACTCTGATACCAGATACAGCCC |
| | | GTCCTTCCAAGGCCAGGTCACCATCTCAGCCGACAAGTCCATCAGCA |
| | | CCGCCTACCTGCAGTGGAGCAGCCTGAAGGCCTCGGACACCGCCAT |
| | | GTATTTCTGTGCGAGACGGAGACAGGGGATCTGGGGTGATGCTCTTG |
| | | ATATCTGGGGCCAAGGGACATTGGTCACCGTCTCTTCA |
| | | (SEQ ID NO: 244) |
| 13E7 | HV-09 | GAGGTGCAGCTGGTGCAGTCTGGAGCAGAGGTGAAAAAGCCCGGGG |
| | | AGTCTCTGATGATCTCCTGTAAGGGTTCTGGATACAGCTTTACCAGC |
| | | TACTGGATCGGCTGGGTGCGCCAGATGCCCGGGAAAGGCCTGGAGT |
| | | GGATGGGGATCATCTATCCTGGAGACTCTGATACCAGATACAGCCC |
| | | GTCCTTCCAAGGCCAGGTCACCATCTCAGCCGACAAGTCCATCAGCA |
| | | CCGCCTACCTGCAGTGGAGCAGCCTGAAGGCCTCGGACACCGCCAT |
| | | GTATTTCTGTGCGAGACGGAGACAGGGGATCTGGGGTGATGCTCTTG |
| | | ATTTCTGGGGCCAAGGGACATTGGTCACCGTCTCTTCA |
| | | (SEQ ID NO: 245) |
| 25F12 | HV-10 | CAGGTGCAGCTACAGCAGTGGGGCGCAGGACTGTTGAAGCCTTCGG |
| | | AGACCCTGTCCCTCACCTGCGCTGTCTATGGTGGGTCCTTCAGTAGT |
| | | TACTACTGGAGCTGGATCCGCCAGCCCCCAGGGAAGGGGCTGGAGT |
| | | GGATTGGGGAAATCAATCATAGTGGAAACACCAACTACAACCCGTC |
| | | CCTCAAGAGTCGAGTCACCATATCAGTAGACACGTCCAAGAACCAG |
| | | TTCTCCCTGAAGCTGAGCTCTGTGACCGCCGCGGACACGGCTGTGTA |
| | | TTACTGTGCGAGAGAGGGGTATTACGATATCTTGACTGGTTATCATG |
| | | ATGCTTTTGATATTTGGGACCAAGGGACAATGGTCACCGTNTTTTCA |
| | | (SEQ ID NO: 246) |
| 32E3 | HV-11 | GAGGTGCAGCTGGTGCAGTCTGGAGCAGAGGTGAAAAAGCCCGGGG |
| | | AGTCTCTGAAGATCTCCTGTAAGGGTTCTGGATACAGCTTTACCAGC |
| | | TACTGGATCGGCTGGGTGCGCCAGATGCCCGGGAAAGGCCTGGAGT |
| | | GGATGGGGATCATCTATCCTGGTGACTCTGATACCAGATACAGCCCG |
| | | TCCTTCCAAGGCCAGGTCACCATCTCAGCCGACAAGTCCATCAGCAC |
| | | CGCCTACCTGCAGTGGAGCACCCTGAAGGCCTCGGACACCGCCATA |
| | | TATTACTGTGCGCGACATGACATTATACCAGCAGCCCCTGGTGCTTT |
| | | TGATATCTGGGGCCAAGGGACAATGGTCACCGTCTCTTCA |
| | | (SEQ ID NO: 247) |
| 24F4 | HV-12 | GAGGTGCAGCTGGTGCAGTCTGGAGCAGAGGTGAAAAAGCCCGGGG |
| | | AGTCTCTGAAGATCTCCTGTAAGGGTTCTGGATACACCTTTACCAGC |
| | | TACTGGATCGGCTGGGTGCGCCAGATGCCCGGGAAAGGCCTGGAGT |
| | | GGATGGGGATCATCTATCCTGGTGACTCTGATACCAGATACAGCCCG |
| | | TCCTTCCAAGGCCAGGTCACCATCTCAGTCGACAAGTCCAGCAGCAC |
| | | CGCCTACCTGCAGTGGAGCAGCCTGAAGGCCTCGGACACCGCCATA |
| | | TATTACTGTACGAGACAGGCCATAGCAGTGACTGGTTTGGGGGGTTT |
| | | CGACCCCTGGGGCCAGGGAACCCTGGTCACCGTCTCCTCA |
| | | (SEQ ID NO: 248) |
| 16B8 | HV-13 | CAGGTTCAGCTGGTGCAGTCTGGAGCTGAGGTGAAGAAGCCTGGGG |
| | | CCTCAGTGAAGGTCTCCTGCAAGGCTTCTGGTTACACCTTTACCAAC |
| | | TATGGTATCAGCTGGGTGCGACAGGCCCCTGGACAAGGGCTTGAGT |
| | | GGATGGGATGGATCAGCGCTTACAATGGTAACACAAACTATGCACA |
| | | GAAGCTCCAGGGCAGAGTCACCATGACCACAGACACATCCACGAGT |
| | | ACAGTCTACATGGAGCTGAGGAGCCTGAGATCTGACGACACGGCCG |
| | | TGTATTACTGTGCGAGACGGGGATACAGCTATGGTTCCTTTGACTAC |
| | | TGGGGCCAGGGAACCCTGGTCACCGTCTCCTCA (SEQ ID NO: 249) |
| 4C5 | HV-14 | GAGGTGCAGCTGGTGCAGTCTGGAGCAGAAGTGAAAAAGCCCGGGG |
| | | AGTCTCTGAAGATCTCCTGTAAGGGTTCTGGACACAGTTTTACCAAC |
| | | TACTGGATCGCCTGGGTGCGCCAGATGCCCGGGAAAGGCCTGGAGT |
| | | GGATGGGGATCATCTATCCTGGTGACTCTGATACCAGATACAGCCCG |
| | | TCCTTCCAAGGCCAGGTCACCATCTCAGCCGACAAGTCCATCAGCAC |
| | | CGCCTACCTGCAGTGGAGCAGCCTGAAGGCCTCGGACACCGCCGTG |
| | | TATTTCTGTGCGAGACAAAGGACGTTTTACTATGATAGTAGTGGTTA |
| | | TTTTGACTACTGGGGCCAGGGAACCCTGGTCACCGTCTCCTCA |
| | | (SEQ ID NO: 250) |
| 6E7 | HV-15 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTACCAGC |
| | | TACTGGATCGCCTGGGTGCGCCAGATGCCCGGGAAAGGCCTGGAGT |
| | | GGATGGGGATCATCTATCCTGGTGACTCTGATACCAGATACAGCCCG |
| | | TCCTTCCAAGGCCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGCGAGACAAAGGACGTTTTATTATGATAGTAGTGATTA |
| | | TTTTGACTACTGGGGCCAGGGAACCCTGGTCACCGTCTCCTCA |
| | | (SEQ ID NO: 251) |
| 5E3 | HV-16 | CAGGTGCAGCTGGTGCAGTCTGGGGCTGAGGTGAAGAAGCCTGGGG |
| | | CCTCAGTGAAGGTCTCCTGCAAGGCTTCTGGATACACCTTCACCGGC |
| | | TACTATATACACTGGGTGCGACAGGCCCCTGGACTAGGGCTTGAGTG |
| | | GATGGGATGGATCAACCCTTACAGTGGTGGCACAACCTCTGCACAG |
| | | AAGTTTCAGGGCAGGGTCACCATGACCAGGGACACGTCCATCAGCT |
| | | CAGCCTACATGGAACTGAGCAGGCTGAGATCTGACGACACGGCCGT |
| | | GTATTACTGTGCGAGAGATGGAGGCTACCTGGCCCTCTACGGTACGG |
| | | ACGTCTGGGGCCAAGGGACCACGGTCACCGTCTCCTCA |
| | | (SEQ ID NO: 252) |
| 4G10 | HV-17 | GAGGTGCAGCTGGTGCAGTCTGGAGCAGAGGTGAAAAAGCCCGGGG |
| | | AGTCTCTGAAGATCTCCTGTAAGGGTTCTGGATACAGCTTTCCCAGC |
| | | TACTGGATCGCCTGGGTGCGCCAGATGCCCGGGAAAGGCCTGGAGT |
| | | GGATGGGGATCATCTATCCTGGTGACTCTGATACCAGATACAGCCCG |
| | | TCCTTCCAAGGCCAGGTCACCATCTCAGCCGACAAGTCCATCAGCAC |
| | | CGCCTTTTTGAAGTGGAGTAGCCTGAAGGCCTCGGACACCGCCATGT |
| | | ATTTCTGTGCGCGACAGGGTATAGAAGTGACTGGTACGGGAGGTTT |
| | | GGACGTCTGGGGCCAAGGGACCACGGTCACCGTCTCCTCA |
| | | (SEQ ID NO: 253) |
| V3 | HV-101 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTGCGAGC |
| | | TACTGGATCGCCTGGGTGCGCCAGATGCCCGGGAAAGGCCTGGAGT |
| | | GGATGGGGATCATCTATCCTGGTGACTCTGATACCAGATACAGCCCG |
| | | TCCTTCCAAGATCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGCGAGAGGGAGGACGTTTTATTATGATAGTAGTGATTA |
| | | TTTTGACTACTGGGGCCAGGGAACCCTGGTCACCGTGTCCTCA |
| | | (SEQ ID NO: 254) |
| V24 | HV-102 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTACCAGC |
| | | TACTGGATTGCCTGGGTGCGCCAGATGCCCGGGAAAGGCCTGGAGT |
| | | GGATGGGGATCATCTATCCTGGTGACTCTGATGTGAGATACAGCCCG |
| | | TCCTTCCAAGGCCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGCGAGATCTAGGACGTTTTATTATGATAGTAGTGATTAT |
| | | TTTGACTACTGGGGCCAGGGAACCCTGGTCACCGTGTCCTCA |
| | | (SEQ ID NO: 255) |
| V27 | HV-103 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTACCAGC |
| | | TACTGGATCGCCTGGGTGCGCCAGATGCCCGGGAAAGGCCTGGAGT |
| | | GGATGGGGATCATCTATCCTGGTGACTCTGATACCAGATACGCTCCG |
| | | TCCTTCCAAGGCCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGTGAGAAGTAGGACGTTTTATTATGATAGTAGTGATTA |
| | | TTTTGACTACTGGGGCCAGGGAACCCTGGTCACCGTGTCCTCA |
| | | (SEQ ID NO: 256) |
| V40 | HV-104 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTGGGAGC |
| | | TACTGGATCGCCTGGGTGCGCCAGATGCCCGGGAAAGGCCTGGAGT |
| | | GGATGGGGATCATCTATCCTGGTGACTCTGATGTTAGATACAGCCCG |
| | | TCCTTCCAAGGCCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGCGAGACAAAGGACGTTTTATTATGATAGTAGTGATTA |
| | | TTCGGACTACTGGGGCCAGGGAACCCTGGTCACCGTGTCCTCA |
| | | (SEQ ID NO: 257) |
| V48 | HV-105 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTGGTAGC |
| | | TACTGGATCGCCTGGGTGCGCCAGATGCCCGGGAAAGGCCTGGAGT |
| | | GGATGGGGATCATCTATCCTGGTGACTCTGATGTGAGATACAGCCCG |
| | | TCCTTCCAAGGCCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGCGAGAATGAGGACGTTTTATTATGATAGTAGTGATTA |
| | | TTTTGACTACTGGGGCCAGGGAACCCTGGTCACCGTGTCCTCA |
| | | (SEQ ID NO: 258) |
| V49 | HV-106 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTAATAGC |
| | | TACTGGATCGCCTGGGTGCGCCAGATGCCCGGGAAAGGCCTGGAGT |
| | | GGATGGGGACGATCTATCCTGGTGACTCTGATACCAGACTGAGCCC |
| | | GTCCTTCCAAGGCCAGGTCACCATCTCAGCCGACAAGTCCATCAGCA |
| | | CCGCCTACCTACAGTGGAGCAGCCTGAAGGCCTCGGACACCGCCAT |
| | | GTATTTCTGTGCGAGAAGTAGGACGTTTTATTATGATAGTAGTGATT |
| | | ATTTTGACTACTGGGGCCAGGGAACCCTGGTCACCGTGTCCTCA |
| | | (SEQ ID NO: 259) |
| V52 | HV-107 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTGAGAGC |
| | | TACTGGATCGCCTGGGTGCGCCAGATGCCCGGGAAAGGCCTGGAGT |
| | | GGATGGGGATCATCTATCCTGGTGACTCTGATACCAGATACAGCCCG |
| | | TCCTTCCAAGGCCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGCGAGAGGGAGGACGTTTTATTATGATAGTAGTGATTA |
| | | TTTTGACTACTGGGGCCAGGGAACCCTGGTCACCGTGTCCTCA |
| | | (SEQ ID NO: 260) |
| V60 | HV-108 | GAGGTGCAGCTGGTGCAGTCTGGAGCAGAGGTGAAAAAGCCCGGGG |
| | | AGTCTCTGAAGATCTCCTGTAAGGGTTCTGGATACCATTTTACCAGC |
| | | TACTGGATCGCCTGGGTGCGCCAGATGCCCGGGAAAGGCCTGGAGT |
| | | GGATGGGGATCATCTATCCTGGTGACTCTGATGTGAGATACAGCCCG |
| | | TCCTTCCAAGGCCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGCGAGACAAAGGACGTTTTATTATGATAGTAGTGATTA |
| | | TAGTGACTACTGGGGCCAGGGAACCCTGGTCACCGTGTCCTCA |
| | | (SEQ ID NO: 261) |
| V73 | HV-109 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTGGTAGC |
| | | TACTGGATCGCCTGGGTGCGCCAGATGCCCGGGAAAGGCCTGGAGT |
| | | GGATGGGGATCATCTATCCTGGTGACTCTGATACCAGATACAGCCCG |
| | | GGGTTCCAAGGCCAGGTCACCATCTCAGCCGACAAGTCCATCAGCA |
| | | CCGCCTACCTACAGTGGAGCAGCCTGAAGGCCTCGGACACCGCCAT |
| | | GTATTTCTGTGCGAGAGGGAGGACGTTTTATTATGATAGTAGTGATT |
| | | ATTTTGACTACTGGGGCCAGGGAACCCTGGTCACCGTGTCCTCA |
| | | (SEQ ID NO: 262) |
| V76 | HV-110 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTGGGAGC |
| | | TACTGGATCGCCTGGGTGCGCCAGATGCCCGGGAAAGGCCTGGAGT |
| | | GGATGGGGATCATCTATCCTGGTGACTCTGATACCAGATACAGCCCG |
| | | GAGTTCCAAGGCCAGGTCACCATCTCAGCCGACAAGTCCATCAGCA |
| | | CCGCCTACCTACAGTGGAGCAGCCTGAAGGCCTCGGACACCGCCAT |
| | | GTATTTCTGTGCGAGACAAAGGACGTTTTATTATGATAGTAGTGATT |
| | | ATAGTGACTACTGGGGCCAGGGAACCCTGGTCACCGTGTCCTCA |
| | | (SEQ ID NO: 263) |
| V84 | HV-111 | GAGGTGCAGCTGGTGCAGTCTGGAGCAGAGGTGAAAAAGCCCGGGG |
| | | AGTCTCTGAAGATCTCCTGTAAGGGTTCTGGATACGGGTTTACCAGC |
| | | TACTGGATCGCCTGGGTGCGCCAGATGCCCGGGAAAGGCCTGGAGT |
| | | GGATGGGGATCATCTATCCTGGTGACAGTGATACCAGATACAGCCC |
| | | GTCCTTCCAAGGCCAGGTCACCATCTCAGCCGACAAGTCCATCAGCA |
| | | CCGCCTACCTACAGTGGAGCAGCCTGAAGGCCTCGGACACCGCCAT |
| | | GTATTTCTGTGCGAGACAAAGGACGTTTTATTATGATAGTAGTGATT |
| | | ATTCGGACTACTGGGGCCAGGGAACCCTGGTCACCGTGTCCTCA |
| | | (SEQ ID NO: 264) |
| V9 | HV-201 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTACCAGC |
| | | TACTGGATCGCCTGGGTGCGCCAGATGCCCGGGAAAGGCCTGGAGT |
| | | GGATGGGGATCATCTATCCTGGTGACTCTGATACCAGATACAGCCCG |
| | | TCCTTCCAAGGCCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGCGAGACAAAGGGGGTTTTATTATGATAGTAGTGATTA |
| | | TTTTGACTACTGGGGCCAGGGAACCCTGGTCACCGTGTCCTCA |
| | | (SEQ ID NO: 319) |
| V10 | HV-15 | GAGGTGCAGCTGGTGCAGTCTGGAGCAGAGGTGAAAAAGCCCGGGG |
| V23 | | AGTCTCTGAAGATCTCCTGTAAGGGTTCTGGATACAGTTTTACCAGC |
| V57 | | TACTGGATCGCCTGGGTGCGCCAGATGCCCGGGAAAGGCCTGGAGT |
| V70 | | GGATGGGGATCATCTATCCTGGTGACTCTGATACCAGATACAGCCCG |
| V83 | | TCCTTCCAAGGCCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGCGAGACAAAGGACGTTTTATTATGATAGTAGTGATTA |
| | | TTTTGACTACTGGGGCCAGGGAACCCTGGTCACCGTGTCCTCA |
| | | (SEQ ID NO: 320) |
| V30 | HV-202 | GAGGTGCAGCTGGTGCAGTCTGGAGCAGAGGTGAAAAAGCCCGGGG |
| | | AGTCTCTGAAGATCTCCTGTAAGGGTTCTGGATCGAGTTTTACCAGC |
| | | TACTGGATCGCCTGGGTGCGCCAGATGCCCGGGAAAGGCCTGGAGT |
| | | GGATGGGGATCATCTATCCTGGTGACTCTGATACCAGATACAGCCCG |
| | | TCCTTCCAAGGCCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGCGAGACAAAGGACGTTTTATTATGATAGTAGTGATTA |
| | | TTTTGACTACTGGGGCCAGGGAACCCTGGTCACCGTGTCCTCA |
| | | (SEQ ID NO: 321) |
| V33 | HV-203 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTACCAGC |
| | | TACTGGATCGCCTGGGTGCGCCAGATGCCCGGGAAAGGCCTGGAGT |
| | | GGATGGGGATCATCTATCCTGGTGACTCTGATACCAGATACAGCCCG |
| | | TCCTTCCAAGGCCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGCGAGACAAAGGACGTTTTATGGGGATAGTAGTGATTA |
| | | TTTTGACTACTGGGGCCAGGGAACCCTGGTCACCGTGTCCTCA |
| | | (SEQ ID NO: 322) |
| V44 | HV-204 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTACCAGC |
| | | TACTGGATCGCCTGGGTGCGCCAGATGCCCGGGAAAGGCCTGGAGT |
| | | GGATGGGGATCATCTATCCTAGTGACTCTGATACCAGATACAGCCCG |
| | | TCCTTCCAAGGCCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGCGAGACAAAGGACGTTTTATTATGATAGTAGTGATTA |
| | | TTTTGACTACTGGGGCCAGGGAACCCTGGTCACCGTGTCCTCA |
| | | (SEQ ID NO: 323) |
| V68 | HV-205 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTACCAGC |
| | | TACTGGATCGCCTGGGTGCGCCAGATGCCCGGGAAAGGCCTGGAGT |
| | | GGATGGGGATCATCTATCCTGGTGACTCTGATACCAGATACAGCCCG |
| | | TCCTTCCAAGGCCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGCGAGACAAAGGACGTTTAGGTATGATAGTAGTGATTA |
| | | TTTTGACTACTGGGGCCAGGGAACCCTGGTCACCGTGTCCTCA |
| | | (SEQ ID NO: 324) |
| V90 | HV-206 | GAGGTGCAGCTGGTGCAGTCTGGAGCAGAGGTGAAAAAGCCCGGGG |
| | | AGTCTCTGAAGATCTCCTGTAAGGGTTCTGGATACAGTTTTACCAGC |
| | | GAGTGGATCGCCTGGGTGCGCCAGATGCCCGGGAAAGGCCTGGAGT |
| | | GGATGGGGATCATCTATCCTGGTGACTCTGATACCAGATACAGCCCG |
| | | TCCTTCCAAGGCCAGGTCACCATCTCAGCCGACAAGTCCATCAGCAC |
| | | CGCCTACCTACAGTGGAGCAGCCTGAAGGCCTCGGACACCGCCATG |
| | | TATTTCTGTGCGAGACAAAGGACGTTTTATTATGATAGTAGTGATTA |
| | | TTTTGACTACTGGGGCCAGGGAACCCTGGTCACCGTGTCCTCA |
| | | (SEQ ID NO: 325) |

In some embodiments, an isolated nucleic acid encoding an anti-TREM2 antibody light chain variable region comprises a sequence that is at least 80% identical, at least 90% identical, at least 95% o identical, or at least 98% o identical to a sequence selected from SEQ ID NOS:208-236 and 313-318. In certain embodiments, an isolated nucleic acid encoding an anti-TREM2 antibody light chain variable region comprises a sequence selected from SEQ ID NOS:208-236 and 313-318. In related embodiments, an isolated nucleic acid encoding an anti-TREM2 antibody heavy chain variable region comprises a sequence that is at least 80% o identical, at least 90% o identical, at least 95% o identical, or at least 98% o identical to a sequence selected from SEQ ID NOS:237-264 and 319-325. In other related embodiments, an isolated nucleic acid encoding an anti-TREM2 antibody heavy chain variable region comprises a sequence selected from SEQ ID NOS:237-264 and 319-325.

In some embodiments, the polynucleotide encodes the full length light chain and full length heavy chain. Exemplary polynucleotide sequences are provided in TABLE A15.

B. U.S. Pat. No. 8,231,878

In some embodiments, the TREM2 agonist is antibody, or an antigen-binding fragment thereof, as described in U.S. Pat. No. 8,231,878, which is incorporated by reference herein, in its entirety. In some embodiments, the TREM2 antibody is monoclonal antibody 29E3, or a fragment, homologue, derivative or variant thereof.

In some embodiments, the TREM2 antigen bind protein comprises a CDRL1, CDRL2, and CDRL3 of the light chain variable region, and a CDRH1, CDRH2, and CDRH3 of the heavy chain variable region of monoclonal antibody 29E3. Monoclonal antibody 29E3 is further described in Bouchon et al., J Exp Med., 2001, 194(8):1111-1122.

In some embodiments, the TREM2 antigen bind protein comprises a light chain variable region and a heavy chain variable region of monoclonal antibody 29E3.

In some embodiments, the TREM2 antigen bind protein is a chimeric antibody containing the light chain variable region and the heavy chain variable region of monoclonal antibody 29E3, and a human heavy chain constant region, such as a human Fc region, or an engineered variant thereof.

In some embodiments, the TREM2 antigen bind protein, e.g., a TREM2 antibody, competes with binding of monoclonal antibody 29E3 to TREM2.

C. U.S. Patent Application Publication No. US2019/0010230A1

In some embodiments, the TREM2 agonist is an antibody, or an antigen-binding fragment thereof, as described in U.S. Patent Application Publication No. US2019/0010230A1 (“the '230 application”), which is incorporated by reference herein, in its entirety.

In some embodiments, the TREM2 binding agent comprises an antibody that comprises a light chain variable domain comprising a CDRL1, CDRL2, and CDRL3 (also referred to as HVR-L1, HVR-L2, and HVR-L3, respectively), and a heavy chain variable domain comprising a CDRH1, CDRH2, and CDRH3 (also referred to as HVR-H1, HVR-H2, and HVR-H3, respectively) disclosed in the '230 application specification. In some embodiments, the TREM2 binding agent comprises an antibody that comprises a light chain variable domain and a heavy chain variable domain disclosed in the '230 application specification.

In some embodiments, the antibody comprises a heavy chain variable domain and a light chain variable domain, wherein the heavy chain variable domain comprises the HVR-H1, HVR-H2, and/or HVR-H3 of the monoclonal antibody Ab52; and/or wherein the light chain variable domain comprises the HVR-L1, HVR-L2, and/orHVR-L3 of the monoclonal antibody Ab52. In some embodiments, the HVR-H1 comprises the amino acid sequence of SEQ ID NO:772. In some embodiments, the HVR-H2 comprises the amino acid sequence of SEQ ID NO:773. In some embodiments, the HVR-H3 comprises the amino acid sequence of SEQ ID NO:774. In some embodiments, the HVR-L1 comprises the amino acid sequence of SEQ ID NO:775. In some embodiments, the HVR-L2 comprises the amino acid sequence of SEQ ID NO:776. In some embodiments, the HVR-L3 comprises the amino acid sequence of SEQ ID NO:777. In some embodiments, the antibody comprises a heavy chain variable domain and a light chain variable domain, wherein the heavy chain variable domain comprises: (a) an HVR-H1 comprising the amino acid sequence of SEQ ID NO:772, or an amino acid sequence with at least about 95% homology to the amino acid sequence of SEQ ID NO:772; (b) an HVR-H2 comprising the amino acid sequence of SEQ ID NO:773, or an amino acid sequence with at least about 95% homology to the amino acid sequence of SEQ ID NO:773; and; and/or (c) an HVR-H3 comprising the amino acid sequence of SEQ ID NO:774, or an amino acid sequence with at least about 95% homology to the amino acid sequence of SEQ ID NO:774; and/or wherein the light chain variable domain comprises: (a) an HVR-L1 comprising the amino acid sequence of SEQ ID NO:775, or an amino acid sequence with at least about 95% homology to the amino acid sequence of SEQ ID NO:775; (b) an HVR-L2 comprising the amino acid sequence of SEQ ID NO:776, or an amino acid sequence with at least about 95% homology to the amino acid sequence of SEQ ID NO:776; and/or (c) an HVR-L3 comprising the amino acid sequence of SEQ ID NO:777, or an amino acid sequence with at least about 95% homology to the amino acid sequence of SEQ ID NO:777. In some embodiments, the antibody comprises a heavy chain variable domain and a light chain variable domain, wherein the heavy chain variable domain comprises the HVR-H1, HVR-H2, and/or HVR-H3 of the monoclonal antibody Ab21; and/or wherein the light chain variable domain comprises the HVR-L1, HVR-L2, and/or HVR-L3 of the monoclonal antibody Ab21. In some embodiments, the HVR-H1 comprises the amino acid sequence of SEQ ID NO:778. In some embodiments, the HVR-H2 comprises the amino acid sequence of SEQ ID NO:779. In some embodiments, the HVR-H3 comprises the amino acid sequence of SEQ ID NO:780. In some embodiments, the HVR-L1 comprises the amino acid sequence of SEQ ID NO:781. In some embodiments, the HVR-L2 comprises the amino acid sequence of SEQ ID NO:782. In some embodiments, the HVR-L3 comprises the amino acid sequence of SEQ ID NO:783. In some embodiments, the antibody comprises a heavy chain variable domain and a light chain variable domain, wherein the heavy chain variable domain comprises: (a) an HVR-H1 comprising the amino acid sequence of SEQ ID NO:778, or an amino acid sequence with at least about 95% homology to the amino acid sequence of SEQ ID NO:778; (b) an HVR-H2 comprising the amino acid sequence of SEQ ID NO:779, or an amino acid sequence with at least about 95% homology to the amino acid sequence of SEQ ID NO:779; and/or (c) an HVR-H3 comprising the amino acid sequence of SEQ ID NO:780, or an amino acid sequence with at least about 95% homology to the amino acid sequence of SEQ ID NO:780, and/or wherein the light chain variable domain comprises: (a) an HVR-L1 comprising the amino acid sequence of SEQ ID NO:781, or an amino acid sequence with at least about 95% homology to the amino acid sequence of SEQ ID NO:781; (b) an HVR-L2 comprising the amino acid sequence of SEQ ID NO:782, or an amino acid sequence with at least about 95% homology to the amino acid sequence of SEQ ID NO:782; and/or (c) an HVR-L3 comprising the amino acid sequence of SEQ ID NO:783, or an amino acid sequence with at least about 95% homology to the amino acid sequence of SEQ ID NO:783.

In some embodiments, the heavy chain variable domain comprises the HVR-H1, HVR-H2, and/or HVR-H3 of the monoclonal antibody Ab52; and/or wherein the light chain variable domain comprises the HVR-L1, HVR-L2, and/or HVR-L3 of the monoclonal antibody Ab52. In some embodiments, the HVR-H1 comprises the amino acid sequence of SEQ ID NO:772. In some embodiments, the HVR-H2 comprises the amino acid sequence of SEQ ID NO:773. In some embodiments, the HVR-H3 comprises the amino acid sequence of SEQ ID NO:774. In some embodiments, the HVR-L1 comprises the amino acid sequence of SEQ ID NO:775. In some embodiments, the HVR-L2 comprises the amino acid sequence of SEQ ID NO:776. In some embodiments, the HVR-L3 comprises the amino acid sequence of SEQ ID NO:777. In some embodiments, the antibody comprises a heavy chain variable domain and a light chain variable domain, wherein the heavy chain variable domain comprises an HVR-H1 comprising the amino acid sequence of SEQ ID NO:772, an HVR-H2 comprising the amino acid sequence of SEQ ID NO:773, and an HVR-H3 comprising the amino acid sequence of SEQ ID NO:774, and/or wherein the light chain variable domain comprises an HVR-L1 comprising the amino acid sequence of SEQ ID NO:775, an HVR-L2 comprising the amino acid sequence of SEQ ID NO:776, and an HVR-L3 comprising the amino acid sequence of SEQ ID NO:777.

In some embodiments, the heavy chain variable domain comprises: (a) an HVR-H1 comprising the amino acid sequence of SEQ ID NO:772, or an amino acid sequence with at least about 95% homology to the amino acid sequence of SEQ ID NO:772; (b) an HVR-H2 comprising the amino acid sequence of SEQ ID NO:773, or an amino acid sequence with at least about 95% homology to the amino acid sequence of SEQ ID NO:773; and; and/or (c) an HVR-H3 comprising the amino acid sequence of SEQ ID NO:774, or an amino acid sequence with at least about 95% homology to the amino acid sequence of SEQ ID NO:774; and/or wherein the light chain variable domain comprises: (a) an HVR-L1 comprising the amino acid sequence of SEQ ID NO:775, or an amino acid sequence with at least about 95% homology to the amino acid sequence of SEQ ID NO:775; (b) an HVR-L2 comprising the amino acid sequence of SEQ ID NO:776, or an amino acid sequence with at least about 95% homology to the amino acid sequence of SEQ ID NO:776; and/or (c) an HVR-L3 comprising the amino acid sequence of SEQ ID NO:777, or an amino acid sequence with at least about 95% homology to the amino acid sequence of SEQ ID NO:777.

In some embodiments, the heavy chain variable domain comprises the HVR-H1, HVR-H2, and/or HVR-H3 of the monoclonal antibody Ab21; and/or wherein the light chain variable domain comprises the HVR-L1, HVR-L2, and/or HVR-L3 of the monoclonal antibody Ab21. In some embodiments, the HVR-H1 comprises the amino acid sequence of SEQ ID NO:778. In some embodiments, the HVR-H2 comprises the amino acid sequence of SEQ ID NO:779. In some embodiments, the HVR-H3 comprises the amino acid sequence of SEQ ID NO:780. In some embodiments, the HVR-L1 comprises the amino acid sequence of SEQ ID NO:781. In some embodiments, the HVR-L2 comprises the amino acid sequence of SEQ ID NO:782. In some embodiments, the HVR-L3 comprises the amino acid sequence of SEQ ID NO:783. In some embodiments, the antibody comprises a heavy chain variable domain and a light chain variable domain, wherein the heavy chain variable domain comprises an HVR-H1 comprising the amino acid sequence of SEQ ID NO:778, an HVR-H2 comprising the amino acid sequence of SEQ ID NO:779, and an HVR-H3 comprising the amino acid sequence of SEQ ID NO:780, and/or wherein the light chain variable domain comprises an HVR-L1 comprising the amino acid sequence of SEQ ID NO:781, an HVR-L2 comprising the amino acid sequence of SEQ ID NO:782, and an HVR-L3 comprising the amino acid sequence of SEQ ID NO:783.

In some embodiments, the heavy chain variable domain comprises: (a) an HVR-H1 comprising the amino acid sequence of SEQ ID NO:778, or an amino acid sequence with at least about 95% homology to the amino acid sequence of SEQ ID NO:778; (b) an HVR-H2 comprising the amino acid sequence of SEQ ID NO:779, or an amino acid sequence with at least about 95% homology to the amino acid sequence of SEQ ID NO:779; and/or (c) an HVR-H3 comprising the amino acid sequence of SEQ ID NO:780, or an amino acid sequence with at least about 95% homology to the amino acid sequence of SEQ ID NO:780, and/or wherein the light chain variable domain comprises: (a) an HVR-L1 comprising the amino acid sequence of SEQ ID NO:781, or an amino acid sequence with at least about 95% homology to the amino acid sequence of SEQ ID NO:781; (b) an HVR-L2 comprising the amino acid sequence of SEQ ID NO:782, or an amino acid sequence with at least about 95% homology to the amino acid sequence of SEQ ID NO:782; and/or (c) an HVR-L3 comprising the amino acid sequence of SEQ ID NO:783, or an amino acid sequence with at least about 95% homology to the amino acid sequence of SEQ ID NO:783.

In some embodiments, the antibody comprises a heavy chain variable domain and a light chain variable domain, wherein the heavy chain variable domain comprises: (a) an HVR-H1 comprising an amino acid sequence selected from the group consisting of SEQ ID NOS:3-24, 772, and 778; an HVR-H2 comprising an amino acid sequence selected from the group consisting of SEQ ID NOS:25-49, 773, and 779; and (c) an HVR-H3 c comprising an amino acid sequence selected from the group consisting of SEQ ID NOS:50-119, 774, and 780; and/or wherein the light chain variable domain comprises: (a) an HVR-L1 c comprising an amino acid sequence selected from the group consisting of SEQ ID NOS: 120-137, 775, and 781; (b) an HVR-L2 comprising an amino acid sequence selected from the group consisting of SEQ ID NOS: 138-152, 776, and 782; and (c) an HVR-L3 comprising an amino acid sequence selected from the group consisting of SEQ ID NOS: 153-236, 777, and 783. In any of the above embodiments, the light chain variable domain and/or heavy chain variable domain comprises an amino acid sequence with at least about 90% homology to the amino acid sequence indicated.

In some embodiments, the antibody is an antibody disclosed in Tables 1A, 1B and 8 and FIGS. 20A and 20B of U.S. Patent Application Publication No. US2019/0010230A1, reproduced below as TABLES C1-C2.

TABLE C1
Kabat heavy chain CDR sequences

| Antibody | | | |
|---|---|---|---|
| Name | CDR L1 | CDR L2 | CDR L3 |
| Ab21 | YSFTTYWIG | IIYPGDSDTRYSPSFQG | ARAGHYDGGHLGMDV |
| | (SEQ ID NO: 778) | (SEQ ID NO: 779) | (SEQ ID NO: 780) |
| Ab52 | YTFTSYYIH | IINPSGGSTSYAQKFQG | AREADDSSGYPLGLDV |
| | (SEQ ID NO: 772) | (SEQ ID NO: 773) | (SEQ ID NO: 774) |

TABLE C2
Kabat light chain CDR sequences

| Antibody | | | |
|---|---|---|---|
| Name | CDR L1 | CDR L2 | CDR L3 |
| Ab21 | RASQSVSSSYLA | GASNRAT | QQDDSAPYT |
| | (SEQ ID NO: 781) | (SEQ ID NO: 782) | (SEQ ID NO: 783) |
| Ab52 | RASQSVSSNLA | GASTRAT | QQVNSLPPT |
| | (SEQ ID NO: 775) | (SEQ ID NO: 776) | (SEQ ID NO: 777) |

TABLE C3
Kabat CDR sequences

| Antibody Name | CDR H1 | CDR H2 | CDR H3 | CDR L1 | CDR L2 | CDR L3 |
|---|---|---|---|---|---|---|
| Ab1 | FTFSSYAMS | VISGSGGSTYYADSVKG | AKGTPTLLFQH | RASQSVSSNLA | GASTRAT | QQLPYWPPT |
| | (SEQ ID NO: 377) | (SEQ ID NO: 399) | (SEQ ID NO: 424) | (SEQ ID NO: 494) | (SEQ ID NO: 512) | (SEQ ID NO: 527) |
| Ab2 | FTFSSSAMS | AISGSGGSTYYAD | AKVPSYDYWSGYSNYYYY | RASQSVGSNLA | GASTRAT | QQYFFYPPT |
| | (SEQ ID NO: 378) | SVKG (SEQ ID NO: 400) | MDV (SEQ ID NO: 425) | (SEQ ID NO: 495) | (SEQ ID NO: 512) | (SEQ ID NO: 528) |
| Ab3 | GTFSSYAIS | GIIPIFGTANYAQK | AREQYHVGMDV | QASQDISNYLN | DASNLAT | QQPFNFPYT |
| | (SEQ ID NO: 379) | FQG (SEQ ID NO: 401) | (SEQ ID NO: 426) | (SEQ ID NO: 496) | (SEQ ID NO: 513) | (SEQ ID NO: 529) |
| Ab4 | GTFSSYAIS | GIIPIFGTASYAQK | ARGVDSIMDY | RASQSVSSNLA | SASTRAT | QQDHDYPFT |
| | (SEQ ID NO: 379) | FQG (SEQ ID NO: 402) | (SEQ ID NO: 427) | (SEQ ID NO: 494) | (SEQ ID NO: 514) | (SEQ ID NO: 530) |
| Ab5 | YTFTSYYIH | IINPSGGSTSYAQK | ARAPQESPYVFDI | RASQSVSSSYLA | GASSRAT | QQYFSSPFT |
| | (SEQ ID NO: 380) | FQG (SEQ ID NO: 403) | (SEQ ID NO: 428) | (SEQ ID NO: 497) | (SEQ ID NO: 515) | (SEQ ID NO: 531) |
| Ab6 | YTFTSYYMH | IINPGGGSTSYAQKFQG | ARGSPTYGYLYDP | RASQSVSSYLA | DASKRAT | QQRVNLPPT |
| | (SEQ ID NO: 381) | (SEQ ID NO: 404) | (SEQ ID NO: 429) | (SEQ ID NO: 498) | (SEQ ID NO: 516) | (SEQ ID NO: 532) |
| Ab7 | YTFTSYYMH | IINPSGGSTTYAQKFQG | ARTSSKERDY | RASQSVSSYLA | DASKRAT | QQRISYPIT |
| | (SEQ ID NO: 381) | (SEQ ID NO: 405) | (SEQ ID NO: 430) | (SEQ ID NO: 498) | (SEQ ID NO: 516) | (SEQ ID NO: 533) |
| Ab8 | GSISSSSYYWG | SISYSGSTYYNPSL | ARGPYRLLLGMDV | RASQSISSYLN | GASSLQS | QQIDDTPIT |
| | (SEQ ID NO: 382) | KS (SEQ ID NO: 406) | (SEQ ID NO: 431) | (SEQ ID NO: 499) | (SEQ ID NO: 517) | (SEQ ID NO: 534) |
| Ab9 | YSFTSYWIG | IIYPGDSDTTYSPS | ARLHISGEVNWFD | RASQSVSSYLA | DASNRAT | QQFSYWPWT |
| | (SEQ ID NO: 383) | FQG (SEQ ID NO: 407) | P (SEQ ID NO: 432) | (SEQ ID NO: 498) | (SEQ ID NO: 518) | (SEQ ID NO: 535) |
| Ab10 | YSFTSNWIG | IIYPGDSDTRYSPS | AREAGYDYGELAF | RASQSVSSSYLA | GASSRAT | QQHDSSPPT |
| | (SEQ ID NO: 384) | FQG (SEQ ID NO: 408) | DI (SEQ ID NO: 433) | (SEQ ID NO: 497) | (SEQ ID NO: 515) | (SEQ ID NO: 536) |
| Abl11 | YSFTTYWIG | IIYPGDSDTRYSPS | ARAGHYDGGHLGMDV | RASQSVSSDYLA | GASSRAT | QQDYSYPWT |
| | (SEQ ID NO: 385) | FQG (SEQ ID NO: 408) | (SEQ ID NO: 434) | (SEQ ID NO: 500) | (SEQ ID NO: 515) | (SEQ ID NO: 537) |
| Ab12 | YSFTSYWIG | IIYPGDSDTRYSPSFQG | ARLGHYSGTVSSYGMDV | RASQSISSYLN | AASSLQS | QQEYAVPYT |
| | (SEQ ID NO: 383) | (SEQ ID NO: 408) | (SEQ ID NO: 435) | (SEQ ID NO: 499) | (SEQ ID NO: 519) | (SEQ ID NO: 538) |
| Ab13 | YTFTSYGIS | WISAYNGNTNYA | ARGPSHYYDLA | RASQSVSSYLA | DASNRAT | QQVSNYPIT |
| | (SEQ ID NO: 386) | QKLQG (SEQ ID NO: 409) | (SEQ ID NO: 436) | (SEQ ID NO: 498) | (SEQ ID NO: 518) | (SEQ ID NO: 539) |
| Ab14 | GSISSGGYYWS | NIYYSGSTVYNPS | ARGLYGYGVLDV | QASQDISNYLN | DASNLET | QQVDNIPPT |
| | (SEQ ID NO: 387) | LKS (SEQ ID NO: 410) | (SEQ ID NO: 437) | (SEQ ID NO: 496) | (SEQ ID NO: 520) | (SEQ ID NO: 540) |
| Ab15 | GSISSGGYYWS | NIYYSGSTVYNPS | ARGLYGYGVLDV | QASQDISNYLN | DASNLET | QQFDTYPT |
| | (SEQ ID NO: 387) | LKS (SEQ ID NO: 410) | (SEQ ID NO: 437) | (SEQ ID NO: 496) | (SEQ ID NO: 520) | (SEQ ID NO: 541) |
| Ab16 | GSISSNSYYWG | SIYYSGSTYYNPS | ARGVLGYGVFDY | QASQDISNYLN | DASNLET | QQFLNFPT |
| | (SEQ ID NO: 388) | LKS (SEQ ID NO: 411) | (SEQ ID NO: 438) | (SEQ ID NO: 496) | (SEQ ID NO: 520) | (SEQ ID NO: 542) |
| Ab17 | GSISSNSYYWG | SIYYSGSTYYNPSL | ARGVLGYGVFDY | QASQDISNYLN | DASNLET | QQFFNFPT |
| | (SEQ ID NO: 388) | KS (SEQ ID NO: 411) | (SEQ ID NO: 438) | (SEQ ID NO: 496) | (SEQ ID NO: 520) | (SEQ ID NO: 543) |
| Ab18 | GSISSYYWS | SIYYSGSTNYNPSL | ARDGGGEYPSGTP | QASQDISNYLN | DASNLET | QQFIDLPFT |
| | (SEQ ID NO: 389) | KS (SEQ ID NO: 412) | FDI (SEQ ID NO: 439) | (SEQ ID NO: 496) | (SEQ ID NO: 520) | (SEQ ID NO: 544) |
| Ab19 | GSISSYYWS | SIYYSGSTNYNPSL | ARDGGGEYPSGTP | QASQDISNYLN | DASNLET | QQYYDLPFT |
| | (SEQ ID NO: 389) | KS (SEQ ID NO: 412) | FDI (SEQ ID NO: 439) | (SEQ ID NO: 496) | (SEQ ID NO: 520) | (SEQ ID NO: 545) |
| Ab20 | GSISSYYWS | SIYYSGSTNYNPSL | ARSGMASFFDY | RASQSVSSDYLA | GASSRAT | QQFSSHPFT |
| | (SEQ ID NO: 389) | KS (SEQ ID NO: 412) | (SEQ ID NO: 440) | (SEQ ID NO: 500) | (SEQ ID NO: 515) | (SEQ ID NO: 546) |
| Ab22 | YSFTTYWIG | IIYPGDSDTRYSPS | ARAGHYDGGHLG | RASQSVSSSYLA | GASSRAT | QQDDRSPYT |
| | (SEQ ID NO: 385) | FQG (SEQ ID NO: 408) | MDV (SEQ ID NO: 434) | (SEQ ID NO: 497) | (SEQ ID NO: 515) | (SEQ ID NO: 547) |
| Ab23 | FTFSSYAMS | AISGSGGSTYYAD | AKLGGHSMDV | KSSQSVLYSSNNKNYLA | WASTRES | QQAYLPPIT |
| | (SEQ ID NO: 377) | SVKG (SEQ ID NO: 400) | (SEQ ID NO: 441) | (SEQ ID NO: 501) | (SEQ ID NO: 521) | (SEQ ID NO: 548) |
| Ab24 | FTFSSYAMS | AISGSGGSTYYAD | AKPLKRGRGFY | RASQSISSYLN | AASSLQS | QQAFSPPPWT |
| | (SEQ ID NO: 377) | SVKG (SEQ ID NO: 400) | (SEQ ID NO: 442) | (SEQ ID NO: 499) | (SEQ ID NO: 519) | (SEQ ID NO: 549) |
| Ab25 | FTFSSYAMS | VISGSGGSTYYAD | AKEGRTITMD | RASQSVSSSYLA | GASSRAT | QQDDRSPT |
| | (SEQ ID NO: 377) | SVKG (SEQ ID NO: 399) | (SEQ ID NO: 443) | (SEQ ID NO: 497) | (SEQ ID NO: 515) | (SEQ ID NO: 550) |
| Ab26 | FTFSSYAMS | VISGSGGSTYYAD | AKDQYSVLDY | RASQSVSSYLA | DASNRAT | QQEFDLPFT |
| | (SEQ ID NO: 377) | SVKG (SEQ ID NO: 399) | (SEQ ID NO: 444) | (SEQ ID NO: 498) | (SEQ ID NO: 518) | (SEQ ID NO: 551) |
| Ab27 | FTFSSYAMS | AISGSGGSTYYAD | AKKYSSRGVYFDY | RASQSVSSYLA | DASNRAT | QQYNNFPPT |
| | (SEQ ID NO: 377) | SVKG (SEQ ID NO: 400) | (SEQ ID NO: 445) | (SEQ ID NO: 498) | (SEQ ID NO: 518) | (SEQ ID NO: 552) |
| Ab28 | FTFSSYAMS | AISGSGGSTYYAD | ARLGGAVGARHVT | RASQSVSSYLA | DASKRAT | QQRYLRPIT |
| | (SEQ ID NO: 377) | SVKG (SEQ ID NO: 400) | YFDY (SEQ ID NO: 446) | (SEQ ID NO: 498) | (SEQ ID NO: 516) | (SEQ ID NO: 553) |
| Ab29 | FTFSSYGMH | VISYDGSNKYYAD | ARGQYYGGSGWF | RASQSVSSSYLA | GASSRAT | QQPGAVPT |
| | (SEQ ID NO: 390) | SVKG (SEQ ID NO: 413) | DP (SEQ ID NO: 447) | (SEQ ID NO: 497) | (SEQ ID NO: 515) | (SEQ ID NO: 554) |
| Ab30 | FTFSSYAMS | AISGSGGSTYYAD | ARLGQEYAYFQH | RASQSISSYLN | GASSLQS | QQVYITPIT |
| | (SEQ ID NO: 377) | SVKG (SEQ ID NO: 400) | (SEQ ID NO: 448) | (SEQ ID NO: 499) | (SEQ ID NO: 517) | (SEQ ID NO: 555) |
| Ab31 | FTFSSYGMH | LIWYDGSNKYYA | ARRRDGYYDEVFD | QASQDISNFLN | DASNLET | QQPVDLPFT |
| | (SEQ ID NO: 390) | DSVKG (SEQ ID NO: 414) | I (SEQ ID NO: 449) | (SEQ ID NO: 502) | (SEQ ID NO: 520) | (SEQ ID NO: 556) |
| Ab32 | FTFSSYAMS | AISGSGGSTYYAD | ARVPKHYVVLDY | RASQSVSSYLA | DASNRAT | QQYSFFPPT |
| | (SEQ ID NO: 377) | SVKG (SEQ ID NO: 400) | (SEQ ID NO: 450) | (SEQ ID NO: 498) | (SEQ ID NO: 518) | (SEQ ID NO: 557) |
| Ab33 | FTFSSYGMH | VISYDGSNKYYAD | ARAGGHLFDY | RASQSVSSYLA | DASNRAT | QQDSSFPPT |
| | (SEQ ID NO: 390) | SVKG (SEQ ID NO: 413) | (SEQ ID NO: 451) | (SEQ ID NO: 498) | (SEQ ID NO: 518) | (SEQ ID NO: 558) |
| Ab34 | FTFSSYGMH | VISYDGSNKYYAD | ARDRGGEYVDFAF | RASQSISSYLN | AASSLQS | QQSDFPPWT |
| | (SEQ ID NO: 390) | SVKG (SEQ ID NO: 413) | DI (SEQ ID NO: 452) | (SEQ ID NO: 499) | (SEQ ID NO: 519) | (SEQ ID NO: 559) |
| Ab35 | FTFSSYAMS | AISGSGGSTYYAD | ARTRSGYGASNYF | RASQSISSYLN | AASSLQS | QQGYSAPIT |
| | (SEQ ID NO: 377) | SVKG (SEQ ID NO: 400) | DY (SEQ ID NO: 453) | (SEQ ID NO: 499) | (SEQ ID NO: 519) | (SEQ ID NO: 560) |
| Ab36 | FTFSTYGMH | VIWYDGSNKYYA | ARGTGAAAASPAF | RASQSVSSYLA | DASNRAT | QQLFDWPT |
| | (SEQ ID NO: 391) | DSVKG (SEQ ID NO: 415) | DI (SEQ ID NO: 454) | (SEQ ID NO: 498) | (SEQ ID NO: 518) | (SEQ ID NO: 561) |
| Ab37 | FTFSSYAMS | AISGSGGSTYYAD | ARVGQYMLGMDV | RASQSVSSYLA | DASNRAT | QQRAFLFT |
| | (SEQ ID NO: 377) | SVKG (SEQ ID NO: 400) | (SEQ ID NO: 455) | (SEQ ID NO: 498) | (SEQ ID NO: 518) | (SEQ ID NO: 562) |
| Ab38 | FTFSTYGMH | VIWYDGSNKYYAD | ARGAPVDYGGIEP | RASQSVSSYLA | DASNRAT | QQIDFLPYT |
| | (SEQ ID NO: 391) | SVKG (SEQ ID NO: 415) | EYFQH (SEQ ID NO: 456) | (SEQ ID NO: 498) | (SEQ ID NO: 518) | (SEQ ID NO: 563) |
| Ab39 | FTFSSYAMS | AISGSGGSTYYAD | AKHYHVGIAFDI | RASQSISSYLN | AASSLQS | QQVYSPPIT |
| | (SEQ ID NO: 377) | SVKG (SEQ ID NO: 400) | (SEQ ID NO: 457) | (SEQ ID NO: 499) | (SEQ ID NO: 519) | (SEQ ID NO: 564) |
| Ab40 | FTFSSYAMS | AISGSGGSTYYAD | ARTRSGYGASNYF | RASQSISSYLN | AASSLQS | QQGYAAPIT |
| | (SEQ ID NO: 377) | SVKG (SEQ ID NO: 400) | DY (SEQ ID NO: 453) | (SEQ ID NO: 499) | (SEQ ID NO: 519) | (SEQ ID NO: 565) |
| Ab41 | FTFSTYAMS | AISGSGGSTYYAD | ARAMARKSVAFDI | RASQSVSSYLA | DASNRAT | QQRYALPIT |
| | (SEQ ID NO: 392) | SVKG (SEQ ID NO: 400) | (SEQ ID NO: 458) | (SEQ ID NO: 498) | (SEQ ID NO: 518) | (SEQ ID NO: 566) |
| Ab42 | FTFSSSAMS | AISGSGGSTYYAD | AKVPSYQRGTAFD | RASQSVSSSYLA | GASSRAT | QQYASPPIT |
| | (SEQ ID NO: 378) | SVKG (SEQ ID NO: 400) | P (SEQ ID NO: 459) | (SEQ ID NO: 497) | (SEQ ID NO: 515) | (SEQ ID NO: 567) |
| Ab43 | FTFSSSAMS | AISGSGGSTYYAD | AKSPAVAGIYRAD | RASQSISRYLN | AASSLQS | QQVYSTPIT |
| | (SEQ ID NO: 378) | SVKG (SEQ ID NO: 400) | Y (SEQ ID NO: 460) | (SEQ ID NO: 503) | (SEQ ID NO: 519) | (SEQ ID NO: 568) |
| Ab44 | FTFSTYGMH | VIWYDGSNKYYAD | ARGTGAAAASPAF | RASQSVSSYLA | DSSNRAT | QQLVHWPT |
| | (SEQ ID NO: 391) | SVKG (SEQ ID NO: 415) | DI (SEQ ID NO: 454) | (SEQ ID NO: 498) | (SEQ ID NO: 522) | (SEQ ID NO: 569) |
| Ab45 | YTFTSYYMH | IINPSGGSTSYAQK | ARGPGYTTALDYY | RASQSVSSNLA | GASTRAT | QQLDDWFT |
| | (SEQ ID NO: 381) | FQG (SEQ ID NO: 403) | YMDV (SEQ ID NO: 461) | (SEQ ID NO: 494) | (SEQ ID NO: 512) | (SEQ ID NO: 570) |
| Ab46 | YTFTSYYMH | IINPSGGSTSYAQK | ARPAKTADY | RASQSVSSYLA | DSSNRAT | QQRSNYPIT |
| | (SEQ ID NO: 381) | FQG (SEQ ID NO: 403) | (SEQ ID NO: 462) | (SEQ ID NO: 498) | (SEQ ID NO: 522) | (SEQ ID NO: 571) |
| Ab47 | YTFTSYYMH | IINPSGGSTTYAQK | ARPGKSMDV | RASQSVSSYLA | DASNRAT | QQRILYPIT |
| | (SEQ ID NO: 381) | FQG (SEQ ID NO: 405) | (SEQ ID NO: 463) | (SEQ ID NO: 498) | (SEQ ID NO: 518) | (SEQ ID NO: 572) |
| Ab48 | YTFTSYYMH | IINPSGGSTTYAQK | ARPGKSMDV | RASQSVSSYLA | DASNRAT | QQRAAYPIT |
| | (SEQ ID NO: 381) | FQG (SEQ ID NO: 405) | (SEQ ID NO: 463) | (SEQ ID NO: 498) | (SEQ ID NO: 518) | (SEQ ID NO: 573) |
| Ab49 | YTFTSYYMH | IINPSGGSTSYAQK | ARPAKTADY | RASQSVSSYLA | DASKRAT | QQRTSHPIT |
| | (SEQ ID NO: 381) | FQG (SEQ ID NO: 403) | (SEQ ID NO: 462) | (SEQ ID NO: 498) | (SEQ ID NO: 516) | (SEQ ID NO: 574) |
| Ab50 | YTFTSYYIH | IINPSGGSTSYAQK | ARAPQESPYVFDI | RASQSVSSSYLA | GASSRAT | QQYAGSPFT |
| | (SEQ ID NO: 380) | FQG (SEQ ID NO: 403) | (SEQ ID NO: 428) | (SEQ ID NO: 497) | (SEQ ID NO: 515) | (SEQ ID NO: 575) |
| Ab51 | YTFTSYYMH | IINPSGGSTSYAQK | ARGVGGQDYYYM | RASQSISSYLN | AASSLQS | QQFDDVFT |
| | (SEQ ID NO: 381) | FQG (SEQ ID NO: 403) | DV (SEQ ID NO: 464) | (SEQ ID NO: 499) | (SEQ ID NO: 519) | (SEQ ID NO: 576) |
| Ab53 | YTFTSYYIH | IINPSGGSTSYAQK | ARAPQESPYVFDI | RASQSVSSSYLA | GASSRAT | QQYVNSPFT |
| | (SEQ ID NO: 380) | FQG (SEQ ID NO: 403) | (SEQ ID NO: 428) | (SEQ ID NO: 497) | (SEQ ID NO: 515) | (SEQ ID NO: 577) |
| Ab54 | YTFTSYYMH | IINPSGGSTSYAQK | ARGPGYTTALDYY | RASQSINSYLN | AASSLQS | QQSDDDPFT |
| | (SEQ ID NO: 381) | FQG (SEQ ID NO: 403) | YMDV (SEQ ID NO: 461) | (SEQ ID NO: 504) | (SEQ ID NO: 519) | (SEQ ID NO: 578) |
| Ab55 | YTFTGSYMH | WINPNSGGTNYAQ | ARGPLYHPMIFDY | RASQSVSSYLA | DASNRAT | QQLSTYPLT |
| | (SEQ ID NO: 393) | KFQG (SEQ ID NO: 416) | (SEQ ID NO: 465) | (SEQ ID NO: 498) | (SEQ ID NO: 518) | (SEQ ID NO: 579) |
| Ab56 | YTFTGYYMH | SINPNSGGTNYAQ | ARASSVDN | RASQSVSSYLA | DASNRAT | QQRSVYPIT |
| | (SEQ ID NO: 394) | KFQG (SEQ ID NO: 417) | (SEQ ID NO: 466) | (SEQ ID NO: 498) | (SEQ ID NO: 518) | (SEQ ID NO: 580) |
| Ab57 | YTFTNYGIS | WISAYNGNTNYA | ARGPTKAYYGSGS | RASQSVSSYLA | DASKRAT | QQVSLFPLT |
| | (SEQ ID NO: 395) | QKLQG (SEQ ID NO: 409) | YVVFDP (SEQ ID NO: 467) | (SEQ ID NO: 498) | (SEQ ID NO: 516) | (SEQ ID NO: 581) |
| Ab58 | YSFTSYWIG | IIYPGDSDTRYSPS | ARLGIYSTGATAF | RASQSISSWLA | DASSLES | LDYNSYSPIT |
| | (SEQ ID NO: 383) | FQG (SEQ ID NO: 408) | DI (SEQ ID NO: 468) | (SEQ ID NO: 505) | (SEQ ID NO: 523) | (SEQ ID NO: 582) |
| Ab59 | YTFTGSYMH | WINPNSGGTNYAQ | ARGGVWYSLFDI | QASQDISNYLN | DASNLET | QQHIALPFT |
| | (SEQ ID NO: 393) | KFQG (SEQ ID NO: 416) | (SEQ ID NO: 469) | (SEQ ID NO: 496) | (SEQ ID NO: 520) | (SEQ ID NO: 583) |
| Ab60 | YTFTGYYMH | WINPNSGGTSYAQ | ARASKMGDD | RASQSVSSYLA | DASKRAT | QQRASMPIT |
| | (SEQ ID NO: 394) | KFQG (SEQ ID NO: 418) | (SEQ ID NO: 470) | (SEQ ID NO: 498) | (SEQ ID NO: 516) | (SEQ ID NO: 584) |
| Ab61 | YTFTSYGIH | WISAYNGNTNYA | ARGGVPRVSYFQH | RASQSVSSYLA | DSSNRAT | QQAFNRPPT |
| | (SEQ ID NO: 396) | QKLQG (SEQ ID NO: 409) | (SEQ ID NO: 471) | (SEQ ID NO: 498) | (SEQ ID NO: 522) | (SEQ ID NO: 585) |
| Ab62 | YSFTSYWIG | IIYPGDSDTRYSPS | ARAGHYDDWSGL | RASQSVSSYLA | DASKRAT | QQSSVHPYT |
| | (SEQ ID NO: 383) | FQG (SEQ ID NO: 408) | GLDV (SEQ ID NO: 472) | (SEQ ID NO: 498) | (SEQ ID NO: 516) | (SEQ ID NO: 586) |
| Ab63 | YTFTSYGIS | WISTYNGNTNYAQ | ARGSGSGYDSWY | RASQGIDSWLA | AASSLQS | QQAYSLPPT |
| | (SEQ ID NO: 386) | KLQG (SEQ ID NO: 419) | D (SEQ ID NO: 473) | (SEQ ID NO: 506) | (SEQ ID NO: 519) | (SEQ ID NO: 587) |
| Ab64 | YSFTSYWIG | IIYPGDSDTRYSPS | ARLGRWSSGSTAF | RASQSVSSNLA | GASTRAT | QQDDDGYT |
| | (SEQ ID NO: 383) | FQG (SEQ ID NO: 408) | DI (SEQ ID NO: 474) | (SEQ ID NO: 494) | (SEQ ID NO: 512) | (SEQ ID NO: 588) |
| Ab65 | YSFTSYWIG | IIYPGDSDTRYSPS | ARLGRKPSGSVAF | RASQSVSSYLA | DASNRAT | QQDYSWPYT |
| | (SEQ ID NO: 383) | FQG (SEQ ID NO: 408) | DI (SEQ ID NO: 475) | (SEQ ID NO: 498) | (SEQ ID NO: 518) | (SEQ ID NO: 589) |
| Ab66 | YTFTGSYMH | WINPNSGGTNYAQ | ARAGHKTHDY | RASQSVSSYLA | DASNRAT | QQRSAYPIT |
| | (SEQ ID NO: 393) | KFQG (SEQ ID NO: 416) | (SEQ ID NO: 476) | (SEQ ID NO: 498) | (SEQ ID NO: 518) | (SEQ ID NO: 590) |
| Ab67 | YTFTSYYMH | IINPSGGSTTYAQK | ARPGKSMDV | RASQSVSSYLA | DASNRAT | QQRSHFPIT |
| | (SEQ ID NO: 381) | FQG (SEQ ID NO: 405) | (SEQ ID NO: 463) | (SEQ ID NO: 498) | (SEQ ID NO: 518) | (SEQ ID NO: 591) |
| Ab68 | FTFSSYGMH | LIWYDGSNKYYA | AKPGSMTDY | RASQSVSSYLA | DASNRAT | QQRANYPIT |
| | (SEQ ID NO: 390) | DSVKG (SEQ ID NO: 414) | (SEQ ID NO: 477) | (SEQ ID NO: 498) | (SEQ ID NO: 518) | (SEQ ID NO: 592) |
| Ab69 | YTFTGSYMH | WINPNSGGTNYAQ | ARAKSVDHDY | RASQSVSSYLA | DASNRAT | QQRADYPIT |
| | (SEQ ID NO: 393) | KFQG (SEQ ID NO: 416) | (SEQ ID NO: 478) | (SEQ ID NO: 498) | (SEQ ID NO: 518) | (SEQ ID NO: 593) |
| Ab70 | YTFTGYYMH | WINPNSGGTSYAQ | ARASKMGDD | RASQSVSSYLA | DASNRAT | QQRSVYPIT |
| | (SEQ ID NO: 394) | KFQG (SEQ ID NO: 418) | (SEQ ID NO: 470) | (SEQ ID NO: 498) | (SEQ ID NO: 518) | (SEQ ID NO: 580) |
| Ab71 | YTFTSYYMH | IINPSGGSTSYAQK | ARDISTHDYDLAF | RASQSVSSSYLA | GASNRAT | QQAGSHPFT |
| | (SEQ ID NO: 381) | FQG (SEQ ID NO: 403) | DI (SEQ ID NO: 479) | (SEQ ID NO: 497) | (SEQ ID NO: 524) | (SEQ ID NO: 594) |
| Ab72 | GSISSYYWS | SIYYSGSTNYNPSL | ARSGTETLFDY | QASQDITNYLN | DASNLET | QQDVNYPPT |
| | (SEQ ID NO: 389) | KS (SEQ ID NO: 412) | (SEQ ID NO: 480) | (SEQ ID NO: 507) | (SEQ ID NO: 520) | (SEQ ID NO: 595) |
| Ab73 | YSFTSYWIG | IIYPGDSDTTYSPS | ARAKMLDDGYAF | RASQSVSSNLA | GASTRAT | QQDDNYPYT |
| | (SEQ ID NO: 383) | FQG (SEQ ID NO: 407) | DI (SEQ ID NO: 481) | (SEQ ID NO: 494) | (SEQ ID NO: 512) | (SEQ ID NO: 596) |
| Ab74 | YTFTGSYMH | WINPNSGGTNYAQ | ARAGHKTHDY | RASQSVSSYLA | DASNRAT | QQRSTFPIT |
| | (SEQ ID NO: 393) | KFQG (SEQ ID NO: 416) | (SEQ ID NO: 476) | (SEQ ID NO: 498) | (SEQ ID NO: 518) | (SEQ ID NO: 597) |
| Ab75 | YTFTGYYMH | WINPNSGGTNYAQ | ARDLGYSSLLALDI | RASQSVSSYLA | DASNRAT | QQVSNYPFT |
| | (SEQ ID NO: 394) | KFQG (SEQ ID NO: 416) | (SEQ ID NO: 482) | (SEQ ID NO: 498) | (SEQ ID NO: 518) | (SEQ ID NO: 598) |
| Ab76 | FTFSSYSMN | SISSSSSYIYYADS | ARGGGRRGDNNW | KSSQSVLYSSNNKNYLA | WASTRES | QQYHDAPIT |
| | (SEQ ID NO: 397) | VKG (SEQ ID NO: 420) | FDP (SEQ ID NO: 483) | (SEQ ID NO: 501) | (SEQ ID NO: 521) | (SEQ ID NO: 599) |
| Ab77 | FTFSSYGMH | VISYDGSNKYYAD | ARGPPHEMDY | KSSQSVLYSSNNKNYLA | WASTRES | QQAYVVPPT |
| | (SEQ ID NO: 390) | SVKG (SEQ ID NO: 413) | (SEQ ID NO: 484) | (SEQ ID NO: 501) | (SEQ ID NO: 521) | (SEQ ID NO: 600) |
| Ab78 | FTFSSYGMH | VIWYDGSNKYYA | ARTPYPWIYFDL | RASQSVSSYLA | DASNRAT | QQADNWPFT |
| | (SEQ ID NO: 390) | DSVKG (SEQ ID NO: 415) | (SEQ ID NO: 485) | (SEQ ID NO: 498) | (SEQ ID NO: 518) | (SEQ ID NO: 601) |
| Ab79 | FTFSSYSMN | YISGSSSTIYYADS | ARGGRRHYGGMD | RSSQSLLHSNGY | LGSHRAS | MQALESPRT |
| | (SEQ ID NO: 397) | VKG (SEQ ID NO: 421) | V (SEQ ID NO: 486) | NYLD (SEQ ID NO: 508) | (SEQ ID NO: 525) | (SEQ ID NO: 602) |
| Ab80 | GTFSSYAIS | GIIPIFGTANYAQK | ARGGGTFWSGSW | RASQSVSSYLA | DASNRAT | QQYVNWPFT |
| | (SEQ ID NO: 379) | FQG (SEQ ID NO: 401) | ALY (SEQ ID NO: 487) | (SEQ ID NO: 498) | (SEQ ID NO: 518) | (SEQ ID NO: 603) |
| Ab81 | GTFSSYAIS | GIIPIFGTANYAQK | ARDSGNYDYWSG | RASQSVSSYLA | DASNRAT | QQSSNWPWT |
| | (SEQ ID NO: 379) | FQG (SEQ ID NO: 401) | ALRY (SEQ ID NO: 488) | (SEQ ID NO: 498) | (SEQ ID NO: 518) | (SEQ ID NO: 604) |
| Ab82 | GSISSGGYYWS | YIYYSGSTVYNPS | ARVSSSWYKA | RASQGISSWLA | AASSLQS | QQASTFPIT |
| | (SEQ ID NO: 387) | LKS (SEQ ID NO: 422) | (SEQ ID NO: 489) | (SEQ ID NO: 509) | (SEQ ID NO: 519) | (SEQ ID NO: 605) |
| Ab83 | GSFSGYYWS | EIDHSGSTKYNPSL | ARVGVVVGRPGYS | RASQGISSWLA | AASSLQS | QQRNSLPLT |
| | (SEQ ID NO: 398) | KS (SEQ ID NO: 423) | AFDI (SEQ ID NO: 490) | (SEQ ID NO: 509) | (SEQ ID NO: 519) | (SEQ ID NO: 606) |
| Ab84 | YTFTSYGIS | WISTYNGNTNYAQ | ARGSGSGYDSWY | RASQSISSYLN | AASSLQS | QQSYDFPIT |
| | (SEQ ID NO: 386) | KLQG (SEQ ID NO: 419) | D (SEQ ID NO: 473) | (SEQ ID NO: 499) | (SEQ ID NO: 519) | (SEQ ID NO: 607) |
| Ab85 | FTFSSYGMH | VIWYDGSNKYYAD | AKDLGGYYGGAA | RASQDISSWLA | AASSLQS | QQEVDYPPLT |
| | (SEQ ID NO: 390) | SVKG (SEQ ID NO: 415) | YGMDV (SEQ ID NO: 491) | (SEQ ID NO: 510) | (SEQ ID NO: 519) | (SEQ ID NO: 608) |
| Ab86 | FTFSSYGMH | VISYDGSNKYYAD | AKDGVYYGLGNW | RASQSISSWLA | KASSLES | QQLNSYSPT |
| | (SEQ ID NO: 390) | SVKG (SEQ ID NO: 413) | FDP (SEQ ID NO: 492) | (SEQ ID NO: 505) | (SEQ ID NO: 526) | (SEQ ID NO: 609) |
| Ab87 | GSISSYYWS | SIYYSGSTNYNPSL | ARHGWDRVGWFD | RASQSVSRYLA | DASNRAT | QQYIFWPPT |
| | (SEQ ID NO: 389) | KS (SEQ ID NO: 412) | P (SEQ ID NO: 493) | (SEQ ID NO: 511) | (SEQ ID NO: 518) | (SEQ ID NO: 610) |

TABLE C4
Heavy chain variable regions

| Ab ID | HC Variable Region Sequences |
|---|---|
| Ab 1 | EVQLLESGGGLVQPGGSLRLSCAASGFTFSSYAMSWVRQAPGKGLEWVSVISGSGGSTY |
| | YADSVKGRFTISRDNSKNTLYLQMNSLRAEDTAVYYCAKGTPTLLFQHWGQGTLVTVSS |
| | (SEQ ID NO: 616) |
| Ab 2 | EVQLLESGGGLVQPGGSLRLSCAASGFTFSSSAMSWVRQAPGKGLEWVSAISGSGGSTYY |
| | ADSVKGRFTISRDNSKNTLYLQMNSLRAEDTAVYYCAKVPSYDYWSGYSNYYYYMDV |
| | WGKGTTVTVSS (SEQ ID NO: 618) |
| Ab 3 | QVQLVQSGAEVKKPGSSVKVSCKASGGTFSSYAISWVRQAPGQGLEWMGGIIPIFGTANY |
| | AQKFQGRVTITADESTSTAYMELSSLRSEDTAVYYCAREQYHVGMDVWGKGTTVTVSS |
| | (SEQ ID NO: 620) |
| Ab 4 | QVQLVQSGAEVKKPGSSVKVSCKASGGTFSSYAISWVRQAPGQGLEWMGGIIPIFGTASY |
| | AQKFQGRVTITADESTSTAYMELSSLRSEDTAVYYCARGVDSIMDYWGQGTLVTVSS |
| | (SEQ ID NO: 622) |
| Ab 5 | QVQLVQSGAEVKKPGASVKVSCKASGYTFTSYYTHWVRQAPGQGLEWMGIINPSGGSTS |
| | YAQKFQGRVTMTRDTSTSTVYMELSSLRSEDTAVYYCARAPQESPYVFDIWGQGTMVTV |
| | SS (SEQ ID NO: 624) |
| Ab 6 | QVQLVQSGAEVKKPGASVKVSCKASGYTFTSYYMHWVRQAPGQGLEWMGIINPGGGST |
| | SYAQKFQGRVTMTRDTSTSTVYMELSSLRSEDTAVYYCARGSPTYGYLYDPWGQGTLVT |
| | VSS (SEQ ID NO: 626) |
| Ab 7 | QVQLVQSGAEVKKPGASVKVSCKASGYTFTSYYMHWVRQAPGQGLEWMGIINPSGGST |
| | TYAQKFQGRVTMTRDTSTSTVYMELSSLRSEDTAVYYCARTSSKERDYWGQGTLVTVSS |
| | (SEQ ID NO: 628) |
| Ab 8 | QLQLQESGPGLVKPSETLSLTCTVSGGSISSSSYYWGWIRQPPGKGLEWIGSISYSGSTYYN |
| | PSLKSRVTISVDTSKNQFSLKLSSVTAADTAVYYCARGPYRLLLGMDVWGQGTTVTVSS |
| | (SEQ ID NO: 630) |
| Ab 9 | EVQLVQSGAEVKKPGESLKISCKGSGYSFTSYWIGWVRQMPGKGLEWMGITYPGDSDTT |
| | YSPSFQGQVTISADKSISTAYLQWSSLKASDTAMYYCARLHISGEVNWFDPWGQGTLVTV |
| | SS (SEQ ID NO: 632) |
| Ab 10 | EVQLVQSGAEVKKPGESLKISCKGSGYSFTSNWIGWVRQMPGKGLEWMGITYPGDSDTR |
| | YSPSFQGQVTISADKSISTAYLQWSSLKASDTAMYYCAREAGYDYGELAFDIWGQGTMV |
| | TVSS (SEQ ID NO: 634) |
| Ab 11 | EVQLVQSGAEVKKPGESLKISCKGSGYSFTTYWIGWVRQMPGKGLEWMGITYPGDSDTR |
| | YSPSFQGQVTISADKSISTAYLQWSSLKASDTAMYYCARAGHYDGGHLGMDVWGQGTT |
| | VTVSS (SEQ ID NO: 636) |
| Ab 12 | EVQLVQSGAEVKKPGESLKISCKGSGYSFTSYWIGWVRQMPGKGLEWMGITYPGDSDTR |
| | YSPSFQGQVTISADKSISTAYLQWSSLKASDTAMYYCARLGHYSGTVSSYGMDVWGQGT |
| | TVTVSS (SEQ ID NO: 638) |
| Ab 13 | QVQLVQSGAEVKKPGASVKVSCKASGYTFTSYGISWVRQAPGQGLEWMGWISAYNGNT |
| | NYAQKLQGRVTMTTDTSTSTAYMELRSLRSDDTAVYYCARGPSHYYDLAWGQGTLVTV |
| | SS (SEQ ID NO: 640) |
| Ab 14 | QVQLQESGPGLVKPSQTLSLTCTVSGGSISSGGYYWSWIRQHPGKGLEWIGNIYYSGSTVY |
| | NPSLKSRVTISVDTSKNQFSLKLSSVTAADTAVYYCARGLYGYGVLDVWGQGTMVTVSS |
| | (SEQ ID NO: 642) |
| Ab 15 | QVQLQESGPGLVKPSQTLSLTCTVSGGSISSGGYYWSWIRQHPGKGLEWIGNIYYSGSTVY |
| | NPSLKSRVTISVDTSKNQFSLKLSSVTAADTAVYYCARGLYGYGVLDVWGQGTMVTVSS |
| | (SEQ ID NO: 642) |
| Ab 16 | QLQLQESGPGLVKPSETLSLTCTVSGGSISSNSYYWGWIRQPPGKGLEWIGSIYYSGSTYY |
| | NPSLKSRVTISVDTSKNQFSLKLSSVTAADTAVYYCARGVLGYGVFDYWGQGTLVTVSS |
| | (SEQ ID NO: 645) |
| Ab 17 | QLQLQESGPGLVKPSETLSLTCTVSGGSISSNSYYWGWIRQPPGKGLEWIGSIYYSGSTYY |
| | NPSLKSRVTISVDTSKNQFSLKLSSVTAADTAVYYCARGVLGYGVFDYWGQGTLVTVSS |
| | (SEQ ID NO: 645) |
| Ab 18 | QVQLQESGPGLVKPSETLSLTCTVSGGSISSYYWSWIRQPPGKGLEWIGSIYYSGSTNYNPS |
| | LKSRVTISVDTSKNQFSLKLSSVTAADTAVYYCARDGGGEYPSGTPFDIWGQGTMVTVSS |
| | (SEQ ID NO: 648) |
| Ab 19 | QVQLQESGPGLVKPSETLSLTCTVSGGSISSYYWSWIRQPPGKGLEWIGSIYYSGSTNYNPS |
| | LKSRVTISVDTSKNQFSLKLSSVTAADTAVYYCARDGGGEYPSGTPFDIWGQGTMVTVSS |
| | (SEQ ID NO: 648) |
| Ab 20 | QVQLQESGPGLVKPSETLSLTCTVSGGSISSYYWSWIRQPPGKGLEWIGSIYYSGSTNYNPS |
| | LKSRVTISVDTSKNQFSLKLSSVTAADTAVYYCARSGMASFFDYWGQGTLVTVSS |
| | (SEQ ID NO: 651) |
| Ab 22 | EVQLVQSGAEVKKPGESLKISCKGSGYSFTTYWIGWVRQMPGKGLEWMGITYPGDSDTR |
| | YSPSFQGQVTISADKSISTAYLQWSSLKASDTAMYYCARAGHYDGGHLGMDVWGQGTT |
| | VTVSS (SEQ ID NO: 636) |
| Ab 23 | EVQLLESGGGLVQPGGSLRLSCAASGFTFSSYAMSWVRQAPGKGLEWVSAISGSGGSTY |
| | YADSVKGRFTISRDNSKNTLYLQMNSLRAEDTAVYYCAKLGGHSMDVWGQGTTVTVSS |
| | (SEQ ID NO: 654) |
| Ab 24 | EVQLLESGGGLVQPGGSLRLSCAASGFTFSSYAMSWVRQAPGKGLEWVSAISGSGGSTY |
| | YADSVKGRFTISRDNSKNTLYLQMNSLRAEDTAVYYCAKPLKRGRGFYWGQGTLVTVSS |
| | (SEQ ID NO: 656) |
| Ab 25 | EVQLLESGGGLVQPGGSLRLSCAASGFTFSSYAMSWVRQAPGKGLEWVSVISGSGGSTY |
| | YADSVKGRFTISRDNSKNTLYLQMNSLRAEDTAVYYCAKEGRTITMDWGQGTLVTVSS |
| | (SEQ ID NO: 658) |
| Ab 26 | EVQLLESGGGLVQPGGSLRLSCAASGFTFSSYAMSWVRQAPGKGLEWVSVISGSGGSTY |
| | YADSVKGRFTISRDNSKNTLYLQMNSLRAEDTAVYYCAKDQYSVLDYWGQGTLVTVSS |
| | (SEQ ID NO: 660) |
| Ab 27 | EVQLLESGGGLVQPGGSLRLSCAASGFTFSSYAMSWVRQAPGKGLEWVSAISGSGGSTY |
| | YADSVKGRFTISRDNSKNTLYLQMNSLRAEDTAVYYCAKKYSSRGVYFDYWGQGTLVT |
| | VSS (SEQ ID NO: 662) |
| Ab 28 | EVQLLESGGGLVQPGGSLRLSCAASGFTFSSYAMSWVRQAPGKGLEWVSAISGSGGSTY |
| | YADSVKGRFTISRDNSKNTLYLQMNSLRAEDTAVYYCARLGGAVGARHVTYFDYWGQG |
| | TLVTVSS (SEQ ID NO: 664) |
| Ab 29 | QVQLVESGGGVVQPGRSLRLSCAASGFTFSSYGMHWVRQAPGKGLEWVAVISYDGSNK |
| | YYADSVKGRFTISRDNSKNTLYLQMNSLRAEDTAVYYCARGQYYGGSGWFDPWGQGTL |
| | VTVSS (SEQ ID NO: 666) |
| Ab 30 | EVQLLESGGGLVQPGGSLRLSCAASGFTFSSYAMSWVRQAPGKGLEWVSAISGSGGSTY |
| | YADSVKGRFTISRDNSKNTLYLQMNSLRAEDTAVYYCARLGQEYAYFQHWGQGTLVTV |
| | SS (SEQ ID NO: 668) |
| Ab 31 | QVQLVESGGGVVQPGRSLRLSCAASGFTFSSYGMHWVRQAPGKGLEWVALIWYDGSNK |
| | YYADSVKGRFTISRDNSKNTLYLQMNSLRAEDTAVYYCARRRDGYYDEVFDIWGQGTM |
| | VTVSS (SEQ ID NO: 670) |
| Ab 32 | EVQLLESGGGLVQPGGSLRLSCAASGFTFSSYAMSWVRQAPGKGLEWVSAISGSGGSTY |
| | YADSVKGRFTISRDNSKNTLYLQMNSLRAEDTAVYYCARVPKHYVVLDYWGQGTLVTV |
| | SS (SEQ ID NO: 672) |
| Ab 33 | QVQLVESGGGVVQPGRSLRLSCAASGFTFSSYGMHWVRQAPGKGLEWVAVISYDGSNK |
| | YYADSVKGRFTISRDNSKNTLYLQMNSLRAEDTAVYYCARAGGHLFDYWGQGTLVTVS |
| | S (SEQ ID NO: 674) |
| Ab 34 | QVQLVESGGGVVQPGRSLRLSCAASGFTFSSYGMHWVRQAPGKGLEWVAVISYDGSNK |
| | YYADSVKGRFTISRDNSKNTLYLQMNSLRAEDTAVYYCARDRGGEYVDFAFDIWGQGT |
| | MVTVSS (SEQ ID NO: 676) |
| Ab 35 | EVQLLESGGGLVQPGGSLRLSCAASGFTFSSYAMSWVRQAPGKGLEWVSAISGSGGSTY |
| | YADSVKGRFTISRDNSKNTLYLQMNSLRAEDTAVYYCARTRSGYGASNYFDYWGQGTL |
| | VTVSS (SEQ ID NO: 678) |
| Ab 36 | QVQLVESGGGVVQPGRSLRLSCAASGFTFSTYGMHWVRQAPGKGLEWVAVIWYDGSNK |
| | YYADSVKGRFTISRDNSKNTLYLQMNSLRAEDTAVYYCARGTGAAAASPAFDIWGQGT |
| | MVTVSS (SEQ ID NO: 680) |
| Ab 37 | EVQLLESGGGLVQPGGSLRLSCAASGFTFSSYAMSWVRQAPGKGLEWVSAISGSGGSTY |
| | YADSVKGRFTISRDNSKNTLYLQMNSLRAEDTAVYYCARVGQYMLGMDVWGQGTTVT |
| | VSS (SEQ ID NO: 682) |
| Ab 38 | QVQLVESGGGVVQPGRSLRLSCAASGFTFSTYGMHWVRQAPGKGLEWVAVIWYDGSNK |
| | YYADSVKGRFTISRDNSKNTLYLQMNSLRAEDTAVYYCARGAPVDYGGIEPEYFQHWGQ |
| | GTLVTVSS (SEQ ID NO: 684) |
| Ab 39 | EVQLLESGGGLVQPGGSLRLSCAASGFTFSSYAMSWVRQAPGKGLEWVSAISGSGGSTY |
| | YADSVKGRFTISRDNSKNTLYLQMNSLRAEDTAVYYCAKHYHVGIAFDIWGQGTMVTVS |
| | S (SEQ ID NO: 686) |
| Ab 40 | EVQLLESGGGLVQPGGSLRLSCAASGFTFSSYAMSWVRQAPGKGLEWVSAISGSGGSTY |
| | YADSVKGRFTISRDNSKNTLYLQMNSLRAEDTAVYYCARTRSGYGASNYFDYWGQGTL |
| | VTVSS (SEQ ID NO: 678) |
| Ab 41 | EVQLLESGGGLVQPGGSLRLSCAASGFTFSTYAMSWVRQAPGKGLEWVSAISGSGGSTY |
| | YADSVKGRFTISRDNSKNTLYLQMNSLRAEDTAVYYCARAMARKSVAFDIWGQGTMVT |
| | VSS (SEQ ID NO: 689) |
| Ab 42 | EVQLLESGGGLVQPGGSLRLSCAASGFTFSSSAMSWVRQAPGKGLEWVSAISGSGGSTYY |
| | ADSVKGRFTISRDNSKNTLYLQMNSLRAEDTAVYYCAKVPSYQRGTAFDPWGQGTLVTV |
| | SS (SEQ ID NO: 691) |
| Ab 43 | EVQLLESGGGLVQPGGSLRLSCAASGFTFSSSAMSWVRQAPGKGLEWVSAISGSGGSTYY |
| | ADSVKGRFTISRDNSKNTLYLQMNSLRAEDTAVYYCAKSPAVAGIYRADYWGQGTLVTV |
| | SS (SEQ ID NO: 693) |
| Ab 44 | QVQLVESGGGVVQPGRSLRLSCAASGFTFSTYGMHWVRQAPGKGLEWVAVIWYDGSNK |
| | YYADSVKGRFTISRDNSKNTLYLQMNSLRAEDTAVYYCARGTGAAAASPAFDIWGQGT |
| | MVTVSS (SEQ ID NO: 680) |
| Ab 45 | QVQLVQSGAEVKKPGASVKVSCKASGYTFTSYYMHWVRQAPGQGLEWMGIINPSGGST |
| | SYAQKFQGRVTMTRDTSTSTVYMELSSLRSEDTAVYYCARGPGYTTALDYYYMDVWGK |
| | GTTVTVSS (SEQ ID NO: 696) |
| Ab 46 | QVQLVQSGAEVKKPGASVKVSCKASGYTFTSYYMHWVRQAPGQGLEWMGIINPSGGST |
| | SYAQKFQGRVTMTRDTSTSTVYMELSSLRSEDTAVYYCARPAKTADYWGQGTLVTVSS |
| | (SEQ ID NO: 698) |
| Ab 47 | QVQLVQSGAEVKKPGASVKVSCKASGYTFTSYYMHWVRQAPGQGLEWMGIINPSGGST |
| | TYAQKFQGRVTMTRDTSTSTVYMELSSLRSEDTAVYYCARPGKSMDVWGQGTTVTVSS |
| | (SEQ ID NO: 700) |
| Ab 48 | QVQLVQSGAEVKKPGASVKVSCKASGYTFTSYYMHWVRQAPGQGLEWMGIINPSGGST |
| | TYAQKFQGRVTMTRDTSTSTVYMELSSLRSEDTAVYYCARPGKSMDVWGQGTTVTVSS |
| | (SEQ ID NO: 700) |
| Ab 49 | QVQLVQSGAEVKKPGASVKVSCKASGYTFTSYYMHWVRQAPGQGLEWMGIINPSGGST |
| | SYAQKFQGRVTMTRDTSTSTVYMELSSLRSEDTAVYYCARPAKTADYWGQGTLVTVSS |
| | (SEQ ID NO: 698) |
| Ab 50 | QVQLVQSGAEVKKPGASVKVSCKASGYTFTSYYIHWVRQAPGQGLEWMGIINPSGGSTS |
| | YAQKFQGRVTMTRDTSTSTVYMELSSLRSEDTAVYYCARAPQESPYVFDIWGQGTMVTV |
| | SS (SEQ ID NO: 624) |
| Ab 51 | QVQLVQSGAEVKKPGASVKVSCKASGYTFTSYYMHWVRQAPGQGLEWMGIINPSGGST |
| | SYAQKFQGRVTMTRDTSTSTVYMELSSLRSEDTAVYYCARGVGGQDYYYMDVWGKGT |
| | TVTVSS (SEQ ID NO: 705) |
| Ab 53 | QVQLVQSGAEVKKPGASVKVSCKASGYTFTSYYIHWVRQAPGQGLEWMGIINPSGGSTS |
| | YAQKFQGRVTMTRDTSTSTVYMELSSLRSEDTAVYYCARAPQESPYVFDIWGQGTMVTV |
| | SS (SEQ ID NO: 624) |
| Ab 54 | QVQLVQSGAEVKKPGASVKVSCKASGYTFTSYYMHWVRQAPGQGLEWMGIINPSGGST |
| | SYAQKFQGRVTMTRDTSTSTVYMELSSLRSEDTAVYYCARGPGYTTALDYYYMDVWGK |
| | GTTVTVSS (SEQ ID NO: 696) |
| Ab 55 | QVQLVQSGAEVKKPGASVKVSCKASGYTFTGSYMHWVRQAPGQGLEWMGWINPNSGG |
| | TNYAQKFQGRVTMTRDTSISTAYMELSRLRSDDTAVYYCARGPLYHPMIFDYWGQGTLV |
| | TVSS (SEQ ID NO: 709) |
| Ab 56 | QVQLVQSGAEVKKPGASVKVSCKASGYTFTGYYMHWVRQAPGQGLEWMGSINPNSGG |
| | TNYAQKFQGRVTMTRDTSISTAYMELSRLRSDDTAVYYCARASSVDNWGQGTLVTVSS |
| | (SEQ ID NO: 711) |
| Ab 57 | QVQLVQSGAEVKKPGASVKVSCKASGYTFTNYGISWVRQAPGQGLEWMGWISAYNGNT |
| | NYAQKLQGRVTMTTDTSTSTAYMELRSLRSDDTAVYYCARGPTKAYYGSGSYVVFDPW |
| | GQGTLVTVSS (SEQ ID NO: 713) |
| Ab 58 | EVQLVQSGAEVKKPGESLKISCKGSGYSFTSYWIGWVRQMPGKGLEWMGITYPGDSDTR |
| | YSPSFQGQVTISADKSISTAYLQWSSLKASDTAMYYCARLGIYSTGATAFDIWGQGTMVT |
| | VSS (SEQ ID NO: 715) |
| Ab 59 | QVQLVQSGAEVKKPGASVKVSCKASGYTFTGSYMHWVRQAPGQGLEWMGWINPNSGG |
| | TNYAQKFQGRVTMTRDTSISTAYMELSRLRSDDTAVYYCARGGVWYSLFDIWGQGTMV |
| | TVSS (SEQ ID NO: 717) |
| Ab 60 | QVQLVQSGAEVKKPGASVKVSCKASGYTFTGYYMHWVRQAPGQGLEWMGWINPNSGG |
| | TSYAQKFQGRVTMTRDTSISTAYMELSRLRSDDTAVYYCARASKMGDDWGQGTLVTVS |
| | S (SEQ ID NO: 719) |
| Ab 61 | QVQLVQSGAEVKKPGASVKVSCKASGYTFTSYGIHWVRQAPGQGLEWMGWISAYNGNT |
| | NYAQKLQGRVTMTTDTSTSTAYMELRSLRSDDTAVYYCARGGVPRVSYFQHWGQGTLV |
| | TVSS (SEQ ID NO: 721) |
| Ab 62 | EVQLVQSGAEVKKPGESLKISCKGSGYSFTSYWIGWVRQMPGKGLEWMGITYPGDSDTR |
| | YSPSFQGQVTISADKSISTAYLQWSSLKASDTAMYYCARAGHYDDWSGLGLDVWGQGT |
| | MVTVSS (SEQ ID NO: 723) |
| Ab 63 | QVQLVQSGAEVKKPGASVKVSCKASGYTFTSYGISWVRQAPGQGLEWMGWISTYNGNT |
| | NYAQKLQGRVTMTTDTSTSTAYMELRSLRSDDTAVYYCARGSGSGYDSWYDWGQGTL |
| | VTVSS (SEQ ID NO: 725) |
| Ab 64 | EVQLVQSGAEVKKPGESLKISCKGSGYSFTSYWIGWVRQMPGKGLEWMGITYPGDSDTR |
| | YSPSFQGQVTISADKSISTAYLQWSSLKASDTAMYYCARLGRWSSGSTAFDIWGQGTMV |
| | TVSS (SEQ ID NO: 727) |
| Ab 65 | EVQLVQSGAEVKKPGESLKISCKGSGYSFTSYWIGWVRQMPGKGLEWMGITYPGDSDTR |
| | YSPSFQGQVTISADKSISTAYLQWSSLKASDTAMYYCARLGRKPSGSVAFDIWGQGTMVT |
| | VSS (SEQ ID NO: 729) |
| Ab 66 | QVQLVQSGAEVKKPGASVKVSCKASGYTFTGSYMHWVRQAPGQGLEWMGWINPNSGG |
| | TNYAQKFQGRVTMTRDTSISTAYMELSRLRSDDTAVYYCARAGHKTHDYWGQGTLVTV |
| | SS (SEQ ID NO: 731) |
| Ab 67 | QVQLVQSGAEVKKPGASVKVSCKASGYTFTSYYMHWVRQAPGQGLEWMGIINPSGGST |
| | TYAQKFQGRVTMTRDTSTSTVYMELSSLRSEDTAVYYCARPGKSMDVWGQGTTVTVSS |
| | (SEQ ID NO: 700) |
| Ab 68 | QVQLVESGGGVVQPGRSLRLSCAASGFTFSSYGMHWVRQAPGKGLEWVALIWYDGSNK |
| | YYADSVKGRFTISRDNSKNTLYLQMNSLRAEDTAVYYCAKPGSMTDYWGQGTLVTVSS |
| | (SEQ ID NO: 734) |
| Ab 69 | QVQLVQSGAEVKKPGASVKVSCKASGYTFTGSYMHWVRQAPGQGLEWMGWINPNSGG |
| | TNYAQKFQGRVTMTRDTSISTAYMELSRLRSDDTAVYYCARAKSVDHDYWGQGTLVTV |
| | SS (SEQ ID NO: 736) |
| Ab 70 | QVQLVQSGAEVKKPGASVKVSCKASGYTFTGYYMHWVRQAPGQGLEWMGWINPNSGG |
| | TSYAQKFQGRVTMTRDTSISTAYMELSRLRSDDTAVYYCARASKMGDDWGQGTLVTVS |
| | S (SEQ ID NO: 719) |
| Ab 71 | QVQLVQSGAEVKKPGASVKVSCKASGYTFTSYYMHWVRQAPGQGLEWMGIINPSGGST |
| | SYAQKFQGRVTMTRDTSTSTVYMELSSLRSEDTAVYYCARDISTHDYDLAFDIWGQGTM |
| | VTVSS (SEQ ID NO: 739) |
| Ab 72 | QVQLQESGPGLVKPSETLSLTCTVSGGSISSYYWSWIRQPPGKGLEWIGSIYYSGSTNYNPS |
| | LKSRVTISVDTSKNQFSLKLSSVTAADTAVYYCARSGTETLFDYWGQGTLVTVSS |
| | (SEQ ID NO: 741) |
| Ab 73 | EVQLVQSGAEVKKPGESLKISCKGSGYSFTSYWIGWVRQMPGKGLEWMGITYPGDSDTT |
| | YSPSFQGQVTISADKSISTAYLQWSSLKASDTAMYYCARAKMLDDGYAFDIWGQGTMVT |
| | VSS (SEQ ID NO: 743) |
| Ab 74 | QVQLVQSGAEVKKPGASVKVSCKASGYTFTGSYMHWVRQAPGQGLEWMGWINPNSGG |
| | TNYAQKFQGRVTMTRDTSISTAYMELSRLRSDDTAVYYCARAGHKTHDYWGQGTLVTV |
| | SS (SEQ ID NO: 731) |
| Ab 75 | QVQLVQSGAEVKKPGASVKVSCKASGYTFTGYYMHWVRQAPGQGLEWMGWINPNSGG |
| | TNYAQKFQGRVTMTRDTSISTAYMELSRLRSDDTAVYYCARDLGYSSLLALDIWGQGTM |
| | VTVSS (SEQ ID NO: 746) |
| Ab 76 | EVQLVESGGGLVKPGGSLRLSCAASGFTFSSYSMNWVRQAPGKGLEWVSSISSSSSYIYY |
| | ADSVKGRFTISRDNAKNSLYLQMNSLRAEDTAVYYCARGGGRRGDNNWFDPWGQGTLV |
| | TVSS (SEQ ID NO: 748) |
| Ab 77 | QVQLVESGGGVVQPGRSLRLSCAASGFTFSSYGMHWVRQAPGKGLEWVAVISYDGSNK |
| | YYADSVKGRFTISRDNSKNTLYLQMNSLRAEDTAVYYCARGPPHEMDYWGQGTLVTVS |
| | S (SEQ ID NO: 750) |
| Ab 78 | QVQLVESGGGVVQPGRSLRLSCAASGFTFSSYGMHWVRQAPGKGLEWVAVIWYDGSNK |
| | YYADSVKGRFTISRDNSKNTLYLQMNSLRAEDTAVYYCARTPYPWIYFDLWGRGTLVTV |
| | SS (SEQ ID NO: 752) |
| Ab 79 | EVQLVESGGGLVQPGGSLRLSCAASGFTFSSYSMNWVRQAPGKGLEWVSYISGSSSTIYY |
| | ADSVKGRFTISRDNAKNSLYLQMNSLRAEDTAVYYCARGGRRHYGGMDVWGQGTTVT |
| | VSS (SEQ ID NO: 754) |
| Ab 80 | QVQLVQSGAEVKKPGSSVKVSCKASGGTFSSYAISWVRQAPGQGLEWMGGIIPIFGTANY |
| | AQKFQGRVTITADESTSTAYMELSSLRSEDTAVYYCARGGGTFWSGSWALYWGQGTLVT |
| | VSS (SEQ ID NO: 756) |
| Ab 81 | QVQLVQSGAEVKKPGSSVKVSCKASGGTFSSYAISWVRQAPGQGLEWMGGIIPIFGTANY |
| | AQKFQGRVTITADESTSTAYMELSSLRSEDTAVYYCARDSGNYDYWSGALRYWGQGTL |
| | VTVSS (SEQ ID NO: 758) |
| Ab 82 | QVQLQESGPGLVKPSQTLSLTCTVSGGSISSGGYYWSWIRQHPGKGLEWIGYIYYSGSTVY |
| | NPSLKSRVTISVDTSKNQFSLKLSSVTAADTAVYYCARVSSSWYKAWGQGTMVTVSS |
| | (SEQ ID NO: 760) |
| Ab 83 | QVQLQQWGAGLLKPSETLSLTCAVYGGSFSGYYWSWIRQPPGKGLEWIGEIDHSGSTKY |
| | NPSLKSRVTISVDTSKNQFSLKLSSVTAADTAVYYCARVGVVVGRPGYSAFDIWGQGTM |
| | VTVSS (SEQ ID NO: 762) |
| Ab 84 | QVQLVQSGAEVKKPGASVKVSCKASGYTFTSYGISWVRQAPGQGLEWMGWISTYNGNT |
| | NYAQKLQGRVTMTTDTSTSTAYMELRSLRSDDTAVYYCARGSGSGYDSWYDWGQGTL |
| | VTVSS (SEQ ID NO: 725) |
| Ab 85 | QVQLVESGGGVVQPGRSLRLSCAASGFTFSSYGMHWVRQAPGKGLEWVAVIWYDGSNK |
| | YYADSVKGRFTISRDNSKNTLYLQMNSLRAEDTAVYYCAKDLGGYYGGAAYGMDVWG |
| | QGTTVTVSS (SEQ ID NO: 765) |
| Ab 86 | QVQLVESGGGVVQPGRSLRLSCAASGFTFSSYGMHWVRQAPGKGLEWVAVISYDGSNK |
| | YYADSVKGRFTISRDNSKNTLYLQMNSLRAEDTAVYYCAKDGVYYGLGNWFDPWGQG |
| | TLVTVSS (SEQ ID NO: 767) |
| Ab 87 | QVQLQESGPGLVKPSETLSLTCTVSGGSISSYYWSWIRQPPGKGLEWIGSIYYSGSTNYNPS |
| | LKSRVTISVDTSKNQFSLKLSSVTAADTAVYYCARHGWDRVGWFDPWGQGTLVTVSS |
| | (SEQ ID NO: 769) |

TABLE C5
Light chain variable regions

| Ab ID | LC Variable Region Sequences |
|---|---|
| Ab 1 | EIVMTQSPATLSVSPGERATLSCRASQSVSSNLAWYQQKPGQAPRLLIYGASTRATGIPAR |
| | FSGSGSGTEFTLTISSLQSEDFAVYYCQQLPYWPPTFGGGTKVEIK (SEQ ID NO: 617) |
| Ab 2 | EIVLTQSPATLSVSPGERATLSCRASQSVGSNLAWYQQKPGQAPRLLIYGASTRATGIPAR |
| | FSGSGSGTEFTLTISSLQSEDFAVYYCQQYFFYPPTFGGGTKVEIK (SEQ ID NO: 619) |
| Ab 3 | DIQMTQSPSSLSASVGDRVTITCQASQDISNYLNWYQQKPGKAPKWYDASNLATGVPSR |
| | FSGSGSGTDFTFTISSLQPEDIATYYCQQPFNFPYTFGGGTKVEIK (SEQ ID NO: 621) |
| Ab 4 | EIVMTQSPATLSVSPGERATLSCRASQSVSSNLAWYQQKPGQAPRLLIYSASTRATGIPAR |
| | FSGSGSGTEFTLTISSLQSEDFAVYYCQQDHDYPFTFGGGTKVEIK (SEQ ID NO: 623) |
| Ab 5 | EIVMTQSPGTLSLSPGERATLSCRASQSVSSSYLAWYQQKPGQAPRLLIYGASSRATGIPD |
| | RFSGSGSGTDFTLTISRLEPEDFAVYYCQQYFSSPFTFGGGTKVEIK (SEQ ID NO: 625) |
| Ab 6 | EIVLTQSPATLSLSPGERATLSCRASQSVSSYLAWYQQKPGQAPRLLIYDASKRATGIPARF |
| | SGSGSGTDFTLTISSLEPEDFAVYYCQQRVNLPPTFGGGTKVEIK (SEQ ID NO: 627) |
| Ab 7 | EIVLTQSPATLSLSPGERATLSCRASQSVSSYLAWYQQKPGQAPRLLIYDASKRATGIPARF |
| | SGSGSGTDFTLTISSLEPEDFAVYYCQQRISYPITFGGGTKVEIK (SEQ ID NO: 629) |
| Ab 8 | DIQMTQSPSSLSASVGDRVTITCRASQSISSYLNWYQQKPGKAPKWYGASSLQSGVPSRF |
| | SGSGSGTDFTLTISSLQPEDFATYYCQQIDDTPITFGGGTKVEIK (SEQ ID NO: 631) |
| Ab 9 | EIVLTQSPATLSLSPGERATLSCRASQSVSSYLAWYQQKPGQAPRLLIYDASNRATGIPARF |
| | SGSGSGTDFTLTISSLEPEDFAVYYCQQFSYWPWTFGGGTKVEIK (SEQ ID NO: 633) |
| Ab 10 | EIVLTQSPGTLSLSPGERATLSCRASQSVSSSYLAWYQQKPGQAPRLLIYGASSRATGIPDR |
| | FSGSGSGTDFTLTISRLEPEDFAVYYCQQHDSSPPTFGGGTKVEIK (SEQ ID NO: 635) |
| Ab 11 | EIVLTQSPGTLSLSPGERATLSCRASQSVSSDYLAWYQQKPGQAPRLLIYGASSRATGIPDR |
| | FSGSGSGTDFTLTISRLEPEDFAVYYCQQDYSYPWTFGGGTKVEIK (SEQ ID NO: 637) |
| Ab 12 | DIQMTQSPSSLSASVGDRVTITCRASQSISSYLNWYQQKPGKAPKLLIYAASSLQSGVPSRF |
| | SGSGSGTDFTLTISSLQPEDFATYYCQQEYAVPYTFGGGTKVEIK (SEQ ID NO: 639) |
| Ab 13 | EIVLTQSPATLSLSPGERATLSCRASQSVSSYLAWYQQKPGQAPRLLIYDASNRATGIPARF |
| | SGSGSGTDFTLTISSLEPEDFAVYYCQQVSNYPITFGGGTKVEIK (SEQ ID NO: 641) |
| Ab 14 | DIQMTQSPSSLSASVGDRVTITCQASQDISNYLNWYQQKPGKAPKWYDASNLETGVPSR |
| | FSGSGSGTDFTFTISSLQPEDIATYYCQQVDNIPPTFGGGTKVEIK (SEQ ID NO: 643) |
| Ab 15 | DIQMTQSPSSLSASVGDRVTITCQASQDISNYLNWYQQKPGKAPKWYDASNLETGVPSR |
| | FSGSGSGTDFTFTISSLQPEDIATYYCQQFDTYPTFGGGTKVEIK (SEQ ID NO: 644) |
| Ab 16 | DIQMTQSPSSLSASVGDRVTITCQASQDISNYLNWYQQKPGKAPKWYDASNLETGVPSR |
| | FSGSGSGTDFTFTISSLQPEDIATYYCQQFLNFPTFGGGTKVEIK (SEQ ID NO: 646) |
| Ab 17 | DIQMTQSPSSLSASVGDRVTITCQASQDISNYLNWYQQKPGKAPKLLIYDASNLETGVPSR |
| | FSGSGSGTDFTFTISSLQPEDIATYYCQQFFNFPTFGGGTKVEIK (SEQ ID NO: 647) |
| Ab 18 | DIQMTQSPSSLSASVGDRVTITCQASQDISNYLNWYQQKPGKAPKLLIYDASNLETGVPSR |
| | FSGSGSGTDFTFTISSLQPEDIATYYCQQFIDLPFTFGGGTKVEIK (SEQ ID NO: 649) |
| Ab 19 | DIQMTQSPSSLSASVGDRVTITCQASQDISNYLNWYQQKPGKAPKLLIYDASNLETGVPSR |
| | FSGSGSGTDFTFTISSLQPEDIATYYCQQYYDLPFTFGGGTKVEIK (SEQ ID NO: 650) |
| Ab 20 | EIVLTQSPGTLSLSPGERATLSCRASQSVSSDYLAWYQQKPGQAPRLLIYGASSRATGIPDR |
| | FSGSGSGTDFTLTISRLEPEDFAVYYCQQFSSHPFTFGGGTKVEIK (SEQ ID NO: 652) |
| Ab 22 | EIVMTQSPGTLSLSPGERATLSCRASQSVSSSYLAWYQQKPGQAPRLLIYGASSRATGIPD |
| | RFSGSGSGTDFTLTISRLEPEDFAVYYCQQDDRSPYTFGGGTKVEIK (SEQ ID NO: 653) |
| Ab 23 | DIVMTQSPDSLAVSLGERATINCKSSQSVLYSSNNKNYLAWYQQKPGQPPKLLISWASTR |
| | ESGVPDRFSGSGSGTDFTLTISSLQAEDVAVYYCQQAYLPPITFGGGTKVEIK |
| | (SEQ ID NO: 655) |
| Ab 24 | DIQMTQSPSSLSASVGDRVTITCRASQSISSYLNWYQQKPGKAPKLLIYAASSLQSGVPSRF |
| | SGSGSGTDFTLTISSLQPEDFATYYCQQAFSPPPWTFGGGTKVEIK (SEQ ID NO: 657) |
| Ab 25 | EIVLTQSPGTLSLSPGERATLSCRASQSVSSSYLAWYQQKPGQAPRLLIYGASSRATGIPDR |
| | FSGSGSGTDFTLTISRLEPEDFAVYYCQQDDRSPTFGGGTKVEIK (SEQ ID NO: 659) |
| Ab 26 | EIVLTQSPATLSLSPGERATLSCRASQSVSSYLAWYQQKPGQAPRLLIYDASNRATGIPA |
| | RFSGSGSGTDFTLTISSLEPEDFAVYYCQQEFDLPFTFGGGTKVEIK (SEQ ID NO: 661) |
| Ab 27 | EIVLTQSPATLSLSPGERATLSCRASQSVSSYLAWYQQKPGQAPRLLIYDASNRATGIPARF |
| | SGSGSGTDFTLTISSLEPEDFAVYYCQQYNNFPPTFGGGTKVEIK (SEQ ID NO: 663) |
| Ab 28 | EIVLTQSPATLSLSPGERATLSCRASQSVSSYLAWYQQKPGQAPRLLIYDASKRATGIPARF |
| | SGSGSGTDFTLTISSLEPEDFAVYYCQQRYLRPITFGGGTKVEIK (SEQ ID NO: 665) |
| Ab 29 | EIVLTQSPGTLSLSPGERATLSCRASQSVSSSYLAWYQQKPGQAPRLLIYGASSRATGIPDR |
| | FSGSGSGTDFTLTISRLEPEDFAVYYCQQPGAVPTFGGGTKVEIK (SEQ ID NO: 667) |
| Ab 30 | DIQMTQSPSSLSASVGDRVTITCRASQSISSYLNWYQQKPGKAPKLLIYGAS SLQSGVPSRF |
| | SGSGSGTDFTLTISSLQPEDFATYYCQQVYITPITFGGGTKVEIK (SEQ ID NO: 669) |
| Ab 31 | DIQLTQSPSSLSASVGDRVTITCQASQDISNFLNWYQQKPGKAPKLLIYDASNLETGVPSRF |
| | SGSGSGTDFTFTISSLQPEDIATYYCQQPVDLPFTFGGGTKVEIK (SEQ ID NO: 671) |
| Ab 32 | EIVLTQSPATLSLSPGERATLSCRASQSVSSYLAWYQQKPGQAPRLLIYDASNRATGIPARF |
| | SGSGSGTDFTLTISSLEPEDFAVYYCQQYSFFPPTFGGGTKVEIK (SEQ ID NO: 673) |
| Ab 33 | EIVLTQSPATLSLSPGERATLSCRASQSVSSYLAWYQQKPGQAPRLLIYDASNRATGIPARF |
| | SGSGSGTDFTLTISSLEPEDFAVYYCQQDSSFPPTFGGGTKVEIK (SEQ ID NO: 675) |
| Ab 34 | DIQMTQSPSSLSASVGDRVTITCRASQSISSYLNWYQQKPGKAPKLLIYAASSLQSGVPSRF |
| | SGSGSGTDFTLTISSLQPEDFATYYCQQSDFPPWTFGGGTKVEIK (SEQ ID NO: 677) |
| Ab 35 | DIQMTQSPSSLSASVGDRVTITCRASQSISSYLNWYQQKPGKAPKLLIYAASSLQSGVPSRF |
| | SGSGSGTDFTLTISSLQPEDFATYYCQQGYSAPITFGGGTKVEIK (SEQ ID NO: 679) |
| Ab 36 | EIVLTQSPATLSLSPGERATLSCRASQSVSSYLAWYQQKPGQAPRLLIYDASNRATGIPARF |
| | SGSGSGTDFTLTISSLEPEDFAVYYCQQLFDWPTFGGGTKVEIK (SEQ ID NO: 681) |
| Ab 37 | EIVLTQSPATLSLSPGERATLSCRASQSVSSYLAWYQQKPGQAPRLLIYDASNRATGIPARF |
| | SGSGSGTDFTLTISSLEPEDFAVYYCQQRAFLFTFGGGTKVEIK (SEQ ID NO: 683) |
| Ab 38 | EIVLTQSPATLSLSPGERATLSCRASQSVSSYLAWYQQKPGQAPRLLIYDASNRATGIPARF |
| | SGSGSGTDFTLTISSLEPEDFAVYYCQQIDFLPYTFGGGTKVEIK (SEQ ID NO: 685) |
| Ab 39 | DIQMTQSPSSLSASVGDRVTITCRASQSISSYLNWYQQKPGKAPKLLIYAASSLQSGVPSRF |
| | SGSGSGTDFTLTISSLQPEDFATYYCQQVYSPPITFGGGTKVEIK (SEQ ID NO: 687) |
| Ab 40 | DIQMTQSPSSLSASVGDRVTITCRASQSISSYLNWYQQKPGKAPKLLIYAASSLQSGVPSRF |
| | SGSGSGTDFTLTISSLQPEDFATYYCQQGYAAPITFGGGTKVEIK (SEQ ID NO: 688) |
| Ab 41 | EIVLTQSPATLSLSPGERATLSCRASQSVSSYLAWYQQKPGQAPRLLIYDASNRATGIPARF |
| | SGSGSGTDFTLTISSLEPEDFTVYYCQQRYALPITFGGGTKVEIK (SEQ ID NO: 690) |
| Ab 42 | EIVLTQSPGTLSLSPGERATLSCRASQSVSSSYLAWYQQKPGQAPRLLIYGASSRATGIPDR |
| | FSGSGSGTDFTLTISRLEPEDFAVYYCQQYASPPITFGGGTKVEIK (SEQ ID NO: 692) |
| Ab 43 | DIQMTQSPSSLSASVGDRVTITCRASQSISRYLNWYQQKPGKAPKLLIYAASSLQSGVPSRF |
| | SGSGSGTDFTLTISSLQPEDFATYYCQQVYSTPITFGGGTKVEIK (SEQ ID NO: 694) |
| Ab 44 | EIVMTQSPATLSLSPGERATLSCRASQSVSSYLAWYQQKPGQAPRLLIYDSSNRATGIPAR |
| | FSGSGSGTDFTLTISSLEPEDFAVYYCQQLVHWPTFGGGTKVEIK (SEQ ID NO: 695) |
| Ab 45 | EIVMTQSPATLSVSPGERATLSCRASQSVSSNLAWYQQKPGQAPRLLIYGASTRATGIPAR |
| | FSGSGSGTEFTLTISSLQSEDFAVYYCQQLDDWFTFGGGTKVEIK (SEQ ID NO: 697) |
| Ab 46 | EIVLTQSPATLSLSPGERATLSCRASQSVSSYLAWYQQKPGQAPRLLIYDSSNRATGIPARF |
| | SGSGSGTDFTLTISSLEPEDFAVYYCQQRSNYPITFGGGTKVEIK (SEQ ID NO: 699) |
| Ab 47 | EIVLTQSPGTLSLSPGERATLSCRASQSVSSYLAWYQQKPGQAPRLLIYDASNRATGIPARF |
| | SGSGSGTDFTLTISSLEPEDFAVYYCQQRILYPITFGGGTKVEIK (SEQ ID NO: 701) |
| Ab 48 | EIVLTQSPATLSLSPGERATLSCRASQSVSSYLAWYQQKPGQAPRLLIYDASNRATGIPARF |
| | SGSGSGTDFTLTISSLEPEDFAVYYCQQRAAYPITFGGGTKVEIK (SEQ ID NO: 702) |
| Ab 49 | EIVLTQSPATLSLSPGERATLSCRASQSVSSYLAWYQQKPGQAPRLLIYDASKRATGIPARF |
| | SGSGSGTDFTLTISSLEPEDFAVYYCQQRTSHPITFGGGTKVEIK (SEQ ID NO: 703) |
| Ab 50 | EIVLTQSPGTLSLSPGERATLSCRASQSVSSSYLAWYQQKPGQAPRLLIYGASSRATGIPDR |
| | FSGSGSGTDFTLTISRLEPEDFAVYYCQQYAGSPFTFGGGTKVEIK (SEQ ID NO: 704) |
| Ab 51 | DIQMTQSPSSLSASVGDRVTITCRASQSISSYLNWYQQKPGKAPKLLIYAASSLQSGVPSRF |
| | SGSGSGTDFTLTISSLQPEDFATYYCQQFDDVFTFGGGTKVEIK (SEQ ID NO: 706) |
| Ab 53 | EIVLTQSPGTLSLSPGERATLSCRASQSVSSSYLAWYQQKPGQAPRLLIYGASSRATGIPDR |
| | FSGSGSGTDFTLTISRLEPEDFAVYYCQQYVNSPFTFGGGTKVEIK (SEQ ID NO: 707) |
| Ab 54 | DIQMTQSPSSLSASVGDRVTITCRASQSINSYLNWYQQKPGKAPKLLIYAASSLQSGVPSR |
| | FSGSGSGTDFTLTISSLQPEDFATYYCQQSDDDPFTFGGGTKVEIK (SEQ ID NO: 708) |
| Ab 55 | EIVLTQSPATLSLSPGERATLSCRASQSVSSYLAWYQQKPGQAPRLLIYDASNRATGIPARF |
| | SGSGSGTDFTLTISSLEPEDFAVYYCQQLSTYPLTFGGGTKVEIK (SEQ ID NO: 710) |
| Ab 56 | EIVMTQSPATLSLSPGERATLSCRASQSVSSYLAWYQQKPGQAPRLLIYDASNRATGIPAR |
| | FSGSGSGTDFTLTISSLEPEDFAVYYCQQRSVYPITFGGGTKVEIK (SEQ ID NO: 712) |
| Ab 57 | EIVLTQSPATLSLSPGERATLSCRASQSVSSYLAWYQQKPGQAPRLLIYDASKRATGIPARF |
| | SGSGSGTDFTLTISSLEPEDFAVYYCQQVSLFPLTFGGGTKVEIK (SEQ ID NO: 714) |
| Ab 58 | DIQMTQSPSTLSASVGDRVTITCRASQSISSWLAWYQQKPGKAPKWYDASSLESGVPSR |
| | FSGSGSGTEFTLTISSLQPDDFATYYCLDYNSYSPITFGGGTKVEIK (SEQ ID NO: 716) |
| Ab 59 | DIQMTQSPSSLSASVGDRVTITCQASQDISNYLNWYQQKPGKAPKLLIYDASNLETGVPSR |
| | FSGSGSGTDFTFTISSLQPEDIATYYCQQHIALPFTFGGGTKVEIK (SEQ ID NO: 718) |
| Ab 60 | EIVLTQSPATLSLSPGERATLSCRASQSVSSYLAWYQQKPGQAPRLLIYDASKRATGIPARF |
| | SGSGSGTDFTLTISSLEPEDFAVYYCQQRASMPITFGGGTKVEIK (SEQ ID NO: 720) |
| Ab 61 | EIVLTQSPATLSLSPGERATLSCRASQSVSSYLAWYQQKPGQAPRLLIYDSSNRATGIPARF |
| | SGSGSGTDFTLTISSLEPEDFAVYYCQQAFNRPPTFGGGTKVEIK (SEQ ID NO: 722) |
| Ab 62 | EIVLTQSPATLSLSPGERATLSCRASQSVSSYLAWYQQKPGQAPRLLIYDASKRATGIPARF |
| | SGSGSGTDFTLTISSLEPEDFAVYYCQQSSVHPYTFGGGTKVEIK (SEQ ID NO: 724) |
| Ab 63 | DIQMTQSPSSVSASVGDRVTITCRASQGIDSWLAWYQQKPGKAPKLLIYAASSLQSGVPSR |
| | FSGSGSGTDFTLTISSLQPEDFATYYCQQAYSLPPTFGGGTKVEIK (SEQ ID NO: 726) |
| Ab 64 | EIVMTQSPATLSVSPGERATLSCRASQSVSSNLAWYQQKPGQAPRLLIYGASTRATGIPAR |
| | FSGSGSGTEFTLTISSLQSEDFAVYYCQQDDDGYTFGGGTKVEIK (SEQ ID NO: 728) |
| Ab 65 | EIVLTQSPATLSLSPGERATLSCRASQSVSSYLAWYQQKPGQAPRLLIYDASNRATGIPARF |
| | SGSGSGTDFTLTISSLEPEDFAVYYCQQDYSWPYTFGGGTKVEIK (SEQ ID NO: 730) |
| Ab 66 | EIVLTQSPGTLSLSPGERATLSCRASQSVSSYLAWYQQKPGQAPRLLIYDASNRATGIPARF |
| | SGSGSGTDFTLTISSLEPEDFAVYYCQQRSAYPITFGGGTKVEIK (SEQ ID NO: 732) |
| Ab 67 | EIVLTQSPATLSLSPGERATLSCRASQSVSSYLAWYQQKPGQAPRLLIYDASNRATGIPARF |
| | SGSGSGTDFTLTISSLEPEDFAVYYCQQRSHFPITFGGGTKVEIK (SEQ ID NO: 733) |
| Ab 68 | EIVLTQSPATLSLSPGERATLSCRASQSVSSYLAWYQQKPGQAPRLLIYDASNRATGIPARF |
| | SGSGSGTDFTLTISSLEPEDFAVYYCQQRANYPITFGGGTKVEIK (SEQ ID NO: 735) |
| Ab 69 | EIVLTQSPATLSLSPGERATLSCRASQSVSSYLAWYQQKPGQAPRLLIYDASNRATGIPARF |
| | SGSGSGTDFTLTISSLEPEDFAVYYCQQRADYPITFGGGTKVEIK (SEQ ID NO: 737) |
| Ab 70 | EIVLTQSPATLSLSPGERATLSCRASQSVSSYLAWYQQKPGQAPRLLIYDASNRATGIPARF |
| | SGSGSGTDFTLTISSLEPEDFAVYYCQQRSVYPITFGGGTKVEIK (SEQ ID NO: 738) |
| Ab 71 | EIVMTQSPGTLSLSPGERATLSCRASQSVSSSYLAWYQQKPGQAPRLLIYGASNRATGIPD |
| | RFSGSGSGTDFTLTISRLEPEDFAVYYCQQAGSHPFTFGGGTKVEIK (SEQ ID NO: 740) |
| Ab 72 | DIQMTQSPSSLSASVGDRVTITCQASQDITNYLNWYQQKPGKAPKWYDASNLETGVPSR |
| | FSGSRSGTDFTFTISSLQPEDIATYYCQQDVNYPPTFGGGTKVEIK (SEQ ID NO: 742) |
| Ab 73 | EIVMTQSPATLSVSPGERATLSCRASQSVSSNLAWYQQKPGQAPRLLIYGASTRATGIPAR |
| | FSGSGSGTEFTLTISSLQSEDFAVYYCQQDDNYPYTFGGGTKVEIK (SEQ ID NO: 744) |
| Ab 74 | EIVLTQSPATLSLSPGERATLSCRASQSVSSYLAWYQQKPGQAPRLLIYDASNRATGIPARF |
| | SGSGSGTDFTLTISSLEPEDFAVYYCQQRSTFPITFGGGTKVEIK (SEQ ID NO: 745) |
| Ab 75 | EIVLTQSPATLSLSPGERATLSCRASQSVSSYLAWYQQKPGQAPRLLIYDASNRATGIPARF |
| | SGSGSGTDFTLTISSLEPEDFAVYYCQQVSNYPFTFGGGTKVEIK (SEQ ID NO: 747) |
| Ab 76 | DIVMTQSPDSLAVSLGERATINCKSSQSVLYSSNNKNYLAWYQQKPGQPPKLLIYWASTR |
| | ESGVPDRFSGSGSGTDFTLTISSLQAEDVAVYYCQQYHDAPITFGGGTKVEIK |
| | (SEQ ID NO: 749) |
| Ab 77 | DIVMTQSPDSLAVSLGERATINCKSSQSVLYSSNNKNYLAWYQQKPGQPPKLLIYWASTR |
| | ESGVPDRFSGSGSGTDFTLTISSLQAEDVAVYYCQQAYVVPPTFGGGTKVEIK |
| | (SEQ ID NO: 751) |
| Ab 78 | EIVLTQSPATLSLSPGERATLSCRASQSVSSYLAWYQQKPGQAPRLLIYDASNRATGIPARF |
| | SGSGSGTDFTLTISSLEPEDFAVYYCQQADNWPFTFGGGTKVEIK (SEQ ID NO: 753) |
| Ab 79 | DIVMTQSPLSLPVTPGEPASISCRSSQSLLHSNGYNYLDWYLQKPGQSPQLLIYLGSHRAS |
| | GVPDRFSGSGSGTDFTLKISRVEAEDVGVYYCMQALESPRTFGGGTKVEIK |
| | (SEQ ID NO: 755) |
| Ab 80 | EIVLTQSPATLSLSPGERATLSCRASQSVSSYLAWYQQKPGQAPRLLIYDASNRATGIPARF |
| | SGSGSGTDFTLTISSLEPEDFAVYYCQQYVNWPFTFGGGTKVEIK (SEQ ID NO: 757) |
| Ab 81 | EIVLTQSPATLSLSPGERATLSCRASQSVSSYLAWYQQKPGQAPRLLIYDASNRATGIPARF |
| | SGSGSGTDFTLTISSLEPEDFAVYYCQQSSNWPWTFGGGTKVEIK (SEQ ID NO: 759) |
| Ab 82 | DIQMTQSPSSVSASVGDRVTITCRASQGISSWLAWYQQKPGKAPKLLIYAASSLQSGVPSR |
| | FSGSGSGTDFTLTISSLQPEDFATYYCQQASTFPITFGGGTKVEIK (SEQ ID NO: 761) |
| Ab 83 | DIQMTQSPSSVSASVGDRVTITCRASQGISSWLAWYQQKPGKAPKLLIYAASSLQSGVPSR |
| | FSGSGSGTDFTLTISSLQPEDFATYYCQQRNSLPLTFGGGTKVEIK (SEQ ID NO: 763) |
| Ab 84 | DIQMTQSPSSLSASVGDRVTITCRASQSISSYLNWYQQKPGKAPKLLIYAASSLQSGVPSRF |
| | SGSGSGTDFTLTISSLQPEDFATYYCQQSYDFPITFGGGTKVEIK (SEQ ID NO: 764) |
| Ab 85 | DIQLTQSPSSVSASVGDRVTITCRASQDISSWLAWYQQKPGKAPKLLIYAASSLQSGVPSR |
| | FSGSGSGTDFTLTISSLQPEDFATYYCQQEVDYPPLTFGGGTKVEIK (SEQ ID NO: 766) |
| Ab 86 | DIQMTQSPSTLSASVGDRVTITCRASQSISSWLAWYQQKPGKAPKWYKASSLESGVPSR |
| | FSGSGSGTEFTLTISSLQPDDFATYYCQQLNSYSPTFGGGTKVEIK (SEQ ID NO: 768) |
| Ab 87 | EIVLTQSPATLSLSPGERATLSCRASQSVSRYLAWYQQKPGQAPRLLIYDASNRATGIPAR |
| | FSGSGSGTDFTLTISSLEPEDFAVYYCQQYIFWPPTFGGGTKVEIK (SEQ ID NO: 770) |

In some embodiments, anti-TREM2 antibodies of the present disclosure comprise (a) a heavy chain variable region comprising at least one, two, or three HVRs selected from HVR-H1, HVR-H2, and HVR-H3 of any one of the antibodies listed in TABLE C3 or selected from Ab1, Ab2, Ab3, Ab4, Ab5, Ab6, Ab7, Ab8, Ab9, Ab100, Ab111, Ab12, Ab13, Ab14, Ab15, Ab16, Ab17, Ab18, Ab19, Ab20, Ab2l, Ab22, Ab23, Ab24, Ab25, Ab26, Ab27, Ab28, Ab29, Ab30, Ab31, Ab32, Ab33, Ab34, Ab35, Ab36, Ab37, Ab38, Ab39, Ab4, Ab4l, Ab42, Ab43, Ab44, Ab45, Ab46, Ab47, Ab48, Ab49, Ab50, Ab51, Ab52, Ab53, Ab54, Ab55, Ab56, Ab57, Ab58, Ab59, Ab60, Ab6l, Ab62, Ab63, Ab64, Ab65, Ab66, Ab67, Ab68, Ab69, Ab70, Ab71, Ab72, Ab73, Ab74, Ab75, Ab76, Ab77, Ab78, Ab79, AbM, Ab81, Ab82, Ab83, Ab84, Ab85, Ab86, and Ab87; and/or (b) a light chain variable region comprising at least one, two, or three HVRs selected from HVR-L1, HVR-L2, and HVR-L3 of any one of the antibodies selected from Ab1, Ab2, Ab3, Ab4, Ab5, Ab6, Ab7, Ab8, Ab9, Ab100, Ab11, Ab12, Ab13, Ab14, Ab15, Ab16, Ab17, Ab18, Ab19, Ab20, Ab2l, Ab22, Ab23, Ab24, Ab25, Ab26, Ab27, Ab28, Ab29, Ab30, Ab3l, Ab32, Ab33, Ab34, Ab35, Ab36, Ab37, Ab38, Ab39, Ab4, Ab4l, Ab42, Ab43, Ab44, Ab45, Ab46, Ab47, Ab48, Ab49, Ab50, Ab51, Ab52, Ab53, Ab54, Ab55, Ab56, Ab57, Ab58, Ab59, Ab60, Ab6l, Ab62, Ab63, Ab64, Ab65, Ab66, Ab67, Ab68, Ab69, Ab70, Ab7l, Ab72, Ab73, Ab74, Ab75, Ab76, Ab77, Ab78, Ab79, Ab80, Ab81, Ab82, Ab83, Ab84, Ab85, Ab86, and Ab87.

In some embodiments, the anti-TREM2 antibody comprises a light chain variable domain and a heavy chain variable region, wherein the light chain variable region comprises a HVR-L1, HVR-L2, and HVR-L3, and the heavy chain variable domain comprises a HVR-H1, HVR-H2, and HVR-H3 of an antibody listed in TABLE C3 or selected from the group consisting of: Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, Ab36, Ab37, Ab38, Ab39, Ab40, Ab41, Ab42, Ab43, Ab44, Ab45, Ab46, Ab47, Ab48, Ab49, Ab50, Ab51, Ab52, Ab53, Ab54, Ab55, Ab56, Ab57, Ab58, Ab59, Ab60, Ab61, Ab62, Ab63, Ab64, Ab65, Ab66, Ab67, Ab68, Ab69, Ab70, Ab71, Ab72, Ab73, Ab74, Ab75, Ab76, Ab77, Ab78, Ab79, Ab80, Ab81, Ab82, Ab83, Ab84, Ab85, Ab86, and Ab87.

In some embodiments, an anti-human TREM2 antibody is an antibody which competes with a monoclonal antibody selected from the group consisting of: Ab1, Ab2, Ab3, Ab4, Ab5, Ab6, Ab7, Ab8, Ab9, Ab10, A11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, Ab36, Ab37, Ab38, Ab39, Ab40, Ab41, Ab42, Ab43, Ab44, Ab45, Ab46, Ab47, Ab48, Ab49, Ab50, Ab51, Ab52, Ab53, Ab54, Ab55, Ab56, Ab57, Ab58, Ab59, Ab60, Ab61, Ab62, Ab63, Ab64, Ab65, Ab66, Ab67, Ab68, Ab69, Ab70, Ab71, Ab72, Ab73, Ab74, Ab75, Ab76, Ab77, Ab78, Ab79, Ab80, Ab81, Ab82, Ab83, Ab84, Ab85, Ab86, and Ab87 for binding to TREM2.

In some embodiments, each of the light chain variable regions disclosed in TABLE C5 and each of the heavy chain variable regions disclosed in TABLE C4 may be attached to the light chain constant regions (EN1) and heavy chain constant regions (EN2) to form complete antibody light and heavy chains, respectively, as further discussed below. Further, each of the generated heavy and light chain sequences may be combined to form a complete antibody structure. It should be understood that the heavy chain and light chain variable regions provided herein can also be attached to other constant domains having different sequences than the exemplary sequences listed herein.

D. PCT Patent Application Publication No. WO2017/062672A1

In some embodiments, the TREM2 agonist is an antibody or an antigen-binding fragment thereof, as described in PCT Patent Application Publication No. WO2017/062672A1 (“the '672 application”), which is incorporated by reference herein, in its entirety.

In some embodiments, the TREM2 binding agent comprises an antibody that comprises a light chain variable domain comprising a CDRL1, CDRL2, and CDRL3 (also referred to as HVR-L1, HVR-L2, and HVR-L3, respectively), and a heavy chain variable domain comprising a CDRH1, CDRH2, and CDRH3 (also referred to as HVR-H1, HVR-H2, and HVR-H3, respectively) disclosed in the '672 application specification. In some embodiments, the TREM2 binding agent comprises an antibody that comprises a light chain variable domain and a heavy chain variable domain disclosed in the '672 application specification.

In some embodiments, the antibody comprises a light chain variable domain and a heavy chain variable domain, wherein the light chain variable domain, or the heavy chain variable domain, or both comprise at least one, two, three, four, five, or six HVRs selected from HVR-L1, HVR-L2, HVR-L3, HVR-H1, HVR-H2, and HVR-H3 such that: (a) the HVR-L1 comprises an amino acid sequence selected from the group consisting of SEQ ID NOS:829-843, 1401, 1510-1514, 1554-1558, and 1646-1648; (b) the HVR-L2 comprises an amino acid sequence selected from the group consisting of SEQ ID NOS:844-853, 1515-1517, and 1559-1563; (c) the HVR-L3 comprises an amino acid sequence selected from the group consisting of SEQ ID NOS:854-867, 1402, 1403, 1518-1522, and 1564-1566; (d) the HVR-H1 comprises an amino acid sequence selected from the group consisting of SEQ ID NOS:868-885, 1404, 1523-1525, 1567-1574, and 1649-1655; (e) the HVR-H2 comprises an amino acid sequence selected from the group consisting of SEQ ID NOS:886-904, 1405-1407, 1526-1528, 1575-1582, 1656-1662, and 1708; or (f) the HVR-H3 comprises an amino acid sequence selected from the group consisting of SEQ ID NOS:905-992, 1408, 1409, 1529, 1530, and 1583-1590. In some embodiments: (a) the HVR-L1 comprises the amino acid sequence of SEQ ID NO:831, the HVR-L2 comprises the amino acid sequence of SEQ ID NO:846, the HVR-L3 comprises the amino acid sequence of SEQ ID NO:856, the HVR-H1 comprises the amino acid sequence of SEQ ID NO:871, the HVR-H2 comprises the amino acid sequence of SEQ ID NO:889, and the HVR-H3 comprises the amino acid sequence of SEQ ID NO:908; (b) the HVR-L1 comprises the amino acid sequence of SEQ ID NO:834, the HVR-L2 comprises the amino acid sequence of SEQ ID NO:848, the HVR-L3 comprises the amino acid sequence of SEQ ID NO:859, the HVR-H1 comprises the amino acid sequence of SEQ ID NO:873, the HVR-H2 comprises the amino acid sequence of SEQ ID NO:891, and the HVR-H3 comprises the amino acid sequence of SEQ ID NO:910; (c) the HVR-L1 comprises the amino acid sequence of SEQ ID NO:831, the HVR-L2 comprises the amino acid sequence of SEQ ID NO:846, the HVR-L3 comprises the amino acid sequence of SEQ ID NO:856, the HVR-H1 comprises the amino acid sequence of SEQ ID NO:871, the HVR-H2 comprises the amino acid sequence of SEQ ID NO:889, and the HVR-H3 comprises the amino acid sequence of SEQ ID NO:908; (d) the HVR-L1 comprises the amino acid sequence of SEQ ID NO:836, the HVR-L2 comprises the amino acid sequence of SEQ ID NO:849, the HVR-L3 comprises the amino acid sequence of SEQ ID NO:855, the HVR-H1 comprises the amino acid sequence of SEQ ID NO:875, the HVR-H2 comprises the amino acid sequence of SEQ ID NO:893, and the HVR-H3 comprises the amino acid sequence of SEQ ID NO:912; (e) the HVR-H1 comprises the amino acid sequence of SEQ ID NO:978, the HVR-H2 comprises the amino acid sequence of SEQ ID NO:896, and the HVR-H3 comprises the amino acid sequence of SEQ ID NO:915; (f) the HVR-L1 comprises the amino acid sequence of SEQ ID NO:839, the HVR-L2 comprises the amino acid sequence of SEQ ID NO:848, the HVR-L3 comprises the amino acid sequence of SEQ ID NO:863, the HVR-H1 comprises the amino acid sequence of SEQ ID NO:880, the HVR-H2 comprises the amino acid sequence of SEQ ID NO:898, and the HVR-H3 comprises the amino acid sequence of SEQ ID NO:917; (g) the HVR-L1 comprises the amino acid sequence of SEQ ID NO:840, the HVR-L2 comprises the amino acid sequence of SEQ ID NO:848, the HVR-L3 comprises the amino acid sequence of SEQ ID NO:868, the HVR-H1 comprises the amino acid sequence of SEQ ID NO:881, the HVR-H2 comprises the amino acid sequence of SEQ ID NO:899, and the HVR-H3 comprises the amino acid sequence of SEQ ID NO:918; (h) the HVR-L1 comprises the amino acid sequence of SEQ ID NO:841, the HVR-L2 comprises the amino acid sequence of SEQ ID NO:852, the HVR-L3 comprises the amino acid sequence of SEQ ID NO:865, the HVR-H1 comprises the amino acid sequence of SEQ ID NO:882, the HVR-H2 comprises the amino acid sequence of SEQ ID NO:900, and the HVR-H3 comprises the amino acid sequence of SEQ ID NO:919; (i) the HVR-L1 comprises the amino acid sequence of SEQ ID NO:842, the HVR-L2 comprises the amino acid sequence of SEQ ID NO:849, the HVR-L3 comprises the amino acid sequence of SEQ ID NO:866, the HVR-H1 comprises the amino acid sequence of SEQ ID NO:883, the HVR-H2 comprises the amino acid sequence of SEQ ID NO:902, and the HVR-H3 comprises the amino acid sequence of SEQ ID NO:920; or (j) the HVR-L1 comprises the amino acid sequence of SEQ ID NO:936, the HVR-L2 comprises the amino acid sequence of SEQ ID NO:849, the HVR-L3 comprises the amino acid sequence of SEQ ID NO:855, the HVR-H1 comprises the amino acid sequence of SEQ ID NO:885, the HVR-H2 comprises the amino acid sequence of SEQ ID NO:904, and the HVR-H3 comprises the amino acid sequence of SEQ ID NO:922. In some embodiments, the antibody comprises a light chain variable domain and a heavy chain variable domain, wherein the light chain variable domain comprises: (a) an HVR-L1 comprising an amino acid sequence selected from the group consisting of SEQ ID NOS:829-843, 1401, 1510-1514, 1554-1558, and 1646-1648, or an amino acid sequence with at least about 90% homology to an amino acid sequence selected from the group consisting of SEQ ID NOS:829-843, 1401, 1510-1514, 1554-1558, and 1646-1648; (b) an HVR-L2 comprising an amino acid sequence selected from the group consisting of SEQ ID NOS:844-853, 1515-1517, and 1559-1563, or an amino acid sequence with at least about 90% homology to an amino acid sequence selected from the group consisting of SEQ ID NOS:844-853, 1515-1517, and 1559-1563; and (c) an HVR-L3 comprising an amino acid sequence selected from the group consisting of SEQ ID NOS:854-867, 1402, 1403, 1518-1522, and 1564-1566, or an amino acid sequence with at least about 90% homology to an amino acid sequence selected from the group consisting of SEQ ID NOS:854-867, 1402, 1403, 1518-1522, and 1564-1566; and wherein the heavy chain variable domain comprises: (a) an HVR-H1 comprising an amino acid sequence selected from the group consisting of SEQ ID NOS:868-885, 1404, 1523-1525, 1567-1574, and 1649-1655, or an amino acid sequence with at least about 90% homology to an amino acid sequence selected from the group consisting of SEQ ID NOS:868-885, 1404, 1523-1525, 1567-1574, and 1649-1655; (b) an HVR-H2 comprising an amino acid sequence selected from the group consisting of SEQ ID NOS:886-904, 1405-1407, 1526-1528, 1575-1582, 1656-1662, and 1708, or an amino acid sequence with at least about 90% homology to an amino acid sequence selected from the group consisting of SEQ ID NOS:886-904, 1405-1407, 1526-1528, 1575-1582, 1656-1662, and 1708; and (c) an HVR-H3 comprising an amino acid sequence selected from the group consisting of SEQ ID NOS:905-992, 1408, 1409, 1529, 1530, and 1583-1590, or an amino acid sequence with at least about 90% homology to an amino acid sequence selected from the group consisting of SEQ ID NOS:905-992, 1408, 1409, 1529, 1530, and 1583-1590. In some embodiments, the antibody comprises a light chain variable domain comprising an amino acid sequence selected from the group consisting of SEQ ID NOS: 1039-1218, 1422-1454, 1499-1509, 1544-1550, 1629-1636, 1641, 1643, 1664, 1669, and 1670; and/or a heavy chain variable domain comprising an amino acid sequence selected from the group consisting of SEQ ID NOS:1219-1400, 1455-1498, 1551-1553, and 1637-1640, 1642-1645, and 1665-1667.

In some embodiments, the antibody comprises a light chain variable domain and a heavy chain variable domain, wherein: (a) the light chain variable domain comprises the amino acid sequence of SEQ ID NO: 1153 and the heavy chain variable domain comprises the amino acid sequence of SEQ ID NO:1341; (b) the light chain variable domain comprises the amino acid sequence of SEQ ID NO: 1670 and the heavy chain variable domain comprises the amino acid sequence of SEQ ID NO: 1341; (c) the light chain variable domain comprises the amino acid sequence of SEQ ID NO:1154 and the heavy chain variable domain comprises the amino acid sequence of SEQ ID NO: 1342; (d) the light chain variable domain comprises the amino acid sequence of SEQ ID NO: 1155 and the heavy chain variable domain comprises the amino acid sequence of SEQ ID NO: 1343; (e) the light chain variable domain comprises the amino acid sequence of SEQ ID NO: 1156 and the heavy chain variable domain comprises the amino acid sequence of SEQ ID NO: 1344; (f) the light chain variable domain comprises the amino acid sequence of SEQ ID NO: 1157 and the heavy chain variable domain comprises the amino acid sequence of SEQ ID NO: 1345; (g) the light chain variable domain comprises the amino acid sequence of SEQ ID NO: 1158 and the heavy chain variable domain comprises the amino acid sequence of SEQ ID NO: 1346; (h) the light chain variable domain comprises the amino acid sequence of SEQ ID NO: 1159 and the heavy chain variable domain comprises the amino acid sequence of SEQ ID NO: 1346; (i) the light chain variable domain comprises the amino acid sequence of SEQ ID NO: 1160 and the heavy chain variable domain comprises the amino acid sequence of SEQ ID NO: 1347; (j) the light chain variable domain comprises the amino acid sequence of SEQ ID NO: 1161 and the heavy chain variable domain comprises the amino acid sequence of SEQ ID NO: 1348; (k) the light chain variable domain comprises the amino acid sequence of SEQ ID NO: 1162 and the heavy chain variable domain comprises the amino acid sequence of SEQ ID NO: 1349; (l) the light chain variable domain comprises the amino acid sequence of SEQ ID NO: 1163 and the heavy chain variable domain comprises the amino acid sequence of SEQ ID NO: 1350; (m) the light chain variable domain comprises the amino acid sequence of SEQ ID NO: 1663 and the heavy chain variable domain comprises the amino acid sequence of SEQ ID NO: 1665; (n) the light chain variable domain comprises the amino acid sequence of SEQ ID NO: 1664 and the heavy chain variable domain comprises the amino acid sequence of SEQ ID NO: 1666; (o) the light chain variable domain comprises the amino acid sequence of SEQ ID NO: 1664 and the heavy chain variable domain comprises the amino acid sequence of SEQ ID NO: 1667; (p) the light chain variable domain comprises the amino acid sequence of SEQ ID NO: 1039 and the heavy chain variable domain comprises the amino acid sequence of SEQ ID NO: 1219; (q) the light chain variable domain comprises the amino acid sequence of SEQ ID NO: 1050 and the heavy chain variable domain comprises the amino acid sequence of SEQ ID NO: 1229; (r) the light chain variable domain comprises the amino acid sequence of SEQ ID NO: 1072 and the heavy chain variable domain comprises the amino acid sequence of SEQ ID NO: 1239; (s) the light chain variable domain comprises the amino acid sequence of SEQ ID NO: 1061 and the heavy chain variable domain comprises the amino acid sequence of SEQ ID NO: 1249; (t) the light chain variable domain comprises the amino acid sequence of SEQ ID NO: 1669 and the heavy chain variable domain comprises the amino acid sequence of SEQ ID NO: 1249; (u) the light chain variable domain comprises the amino acid sequence of SEQ ID NO: 1083 and the heavy chain variable domain comprises the amino acid sequence of SEQ ID NO: 1259; (v) the light chain variable domain comprises the amino acid sequence of SEQ ID NO: 1094 and the heavy chain variable domain comprises the amino acid sequence of SEQ ID NO: 1269; (w) the light chain variable domain comprises the amino acid sequence of SEQ ID NO: 1105 and the heavy chain variable domain comprises the amino acid sequence of SEQ ID NO: 1279; (x) the light chain variable domain comprises the amino acid sequence of SEQ ID NO: 1106 and the heavy chain variable domain comprises the amino acid sequence of SEQ ID NO: 1280; (y) the light chain variable domain comprises the amino acid sequence of SEQ ID NO: 1107 and the heavy chain variable domain comprises the amino acid sequence of SEQ ID NO:1281; (z) the light chain variable domain comprises the amino acid sequence of SEQ ID NO: 1118 and the heavy chain variable domain comprises the amino acid sequence of SEQ ID NO: 1249; (aa) the light chain variable domain comprises the amino acid sequence of SEQ ID NO: 1119 and the heavy chain variable domain comprises the amino acid sequence of SEQ ID NO:1291; (bb) the light chain variable domain comprises the amino acid sequence of SEQ ID NO: 1130 and the heavy chain variable domain comprises the amino acid sequence of SEQ ID NO:1281; (cc) the light chain variable domain comprises the amino acid sequence of SEQ ID NO: 1499 and the heavy chain variable domain comprises the amino acid sequence of SEQ ID NO:1301; (dd) the light chain variable domain comprises the amino acid sequence of SEQ ID NO:1131 and the heavy chain variable domain comprises the amino acid sequence of SEQ ID NO: 1311; (ee) the light chain variable domain comprises the amino acid sequence of SEQ ID NO: 1142 and the heavy chain variable domain comprises the amino acid sequence of SEQ ID NO:1331; (ff) the light chain variable domain comprises the amino acid sequence of SEQ ID NO: 1164 and the heavy chain variable domain comprises the amino acid sequence of SEQ ID NO:1351; (gg) the light chain variable domain comprises the amino acid sequence of SEQ ID NO: 1175 and the heavy chain variable domain comprises the amino acid sequence of SEQ ID NO:1455; (hh) the light chain variable domain comprises the amino acid sequence of SEQ ID NO:1185 and the heavy chain variable domain comprises the amino acid sequence of SEQ ID NO:1361; (ii) the light chain variable domain comprises the amino acid sequence of SEQ ID NO: 1216 and the heavy chain variable domain comprises the amino acid sequence of SEQ ID NO:1371; (jj) the light chain variable domain comprises the amino acid sequence of SEQ ID NO: 1217 and the heavy chain variable domain comprises the amino acid sequence of SEQ ID NO:1381; (kk) the light chain variable domain comprises the amino acid sequence of SEQ ID NO: 1218 and the heavy chain variable domain comprises the amino acid sequence of SEQ ID NO: 1391; (ll) the light chain variable domain comprises the amino acid sequence of SEQ ID NO: 1544 and the heavy chain variable domain comprises the amino acid sequence of SEQ ID NO:1551; (mm) the light chain variable domain comprises the amino acid sequence of SEQ ID NO: 1629 and the heavy chain variable domain comprises the amino acid sequence of SEQ ID NO:1551; (nn) the light chain variable domain comprises the amino acid sequence of SEQ ID NO: 1545 and the heavy chain variable domain comprises the amino acid sequence of SEQ ID NO: 1552; (oo) the light chain variable domain comprises the amino acid sequence of SEQ ID NO: 1546 and the heavy chain variable domain comprises the amino acid sequence of SEQ ID NO:1551; (pp) the light chain variable domain comprises the amino acid sequence of SEQ ID NO: 1546 and the heavy chain variable domain comprises the amino acid sequence of SEQ ID NO:1637; (qq) the light chain variable domain comprises the amino acid sequence of SEQ ID NO: 1547 and the heavy chain variable domain comprises the amino acid sequence of SEQ ID NO:1551; (rr) the light chain variable domain comprises the amino acid sequence of SEQ ID NO: 1548 and the heavy chain variable domain comprises the amino acid sequence of SEQ ID NO: 1553; (ss) the light chain variable domain comprises the amino acid sequence of SEQ ID NO: 1630 and the heavy chain variable domain comprises the amino acid sequence of SEQ ID NO: 1638; (tt) the light chain variable domain comprises the amino acid sequence of SEQ ID NO: 1631 and the heavy chain variable domain comprises the amino acid sequence of SEQ ID NO: 1553; (uu) the light chain variable domain comprises the amino acid sequence of SEQ ID NO: 1549 and the heavy chain variable domain comprises the amino acid sequence of SEQ ID NO:1551; (vv) the light chain variable domain comprises the amino acid sequence of SEQ ID NO: 1632 and the heavy chain variable domain comprises the amino acid sequence of SEQ ID NO: 1639; (ww) the light chain variable domain comprises the amino acid sequence of SEQ ID NO: 1549 and the heavy chain variable domain comprises the amino acid sequence of SEQ ID NO: 1640; (xx) the light chain variable domain comprises the amino acid sequence of SEQ ID NO:1550 and the heavy chain variable domain comprises the amino acid sequence of SEQ ID NO:1551; (yy) the light chain variable domain comprises the amino acid sequence of SEQ ID NO: 1633 and the heavy chain variable domain comprises the amino acid sequence of SEQ ID NO:1551; (zz) the light chain variable domain comprises the amino acid sequence of SEQ ID NO: 1634 and the heavy chain variable domain comprises the amino acid sequence of SEQ ID NO: 1642; (aaa) the light chain variable domain comprises the amino acid sequence of SEQ ID NO: 1635 and the heavy chain variable domain comprises the amino acid sequence of SEQ ID NO: 1644; or (bbb) the light chain variable domain comprises the amino acid sequence of SEQ ID NO: 1636 and the heavy chain variable domain comprises the amino acid sequence of SEQ ID NO:1645. In any of the above embodiments, the light chain variable domain and/or heavy chain variable domain comprises an amino acid sequence with at least about 90% homology to the amino acid sequence indicated.

In some embodiments, the antibody is an antibody disclosed in Tables 2A, 2B, 3A, 3B, 4A, 4B, 7A and 7B of PCT Patent Application Publication No. WO2017/062672A1, reproduced below as TABLES D1-D8.

TABLE D1
EU or Kabat light chain HVR sequences

| Ab ID | HVRL1 | HVRL2 | HVRL3 |
|---|---|---|---|
| 4D11 | RASENIYSFLA | NSKTFAE | QHHYGTPPWT |
| | (SEQ ID NO: 829) | (SEQ ID NO: 844) | (SEQ ID NO: 854) |
| 78C5 | RASENIYSFLA | NSKTFAE | QHHYGTPPWT |
| | (SEQ ID NO: 829) | (SEQ ID NO: 844) | (SEQ ID NO: 854) |
| 6G12 | KSSQSLLYSSNQKNCLA | WAFTRES | QQYYSYPLT |
| | (SEQ ID NO: 830) | (SEQ ID NO: 845) | (SEQ ID NO: 855) |
| 8F11 | KSSQSLLYSSNQKNCLA | LVSKLDS | MQGTHFPLT |
| | (SEQ ID NO: 830) | (SEQ ID NO: 846) | (SEQ ID NO: 856) |
| 8E10 | KSSQSLLDSDGKTYLN | LVSKLDS | WQGTHFPYT |
| | (SEQ ID NO: 832) | (SEQ ID NO: 846) | (SEQ ID NO: 857) |
| 7E5 | KSSQSLLYSNGKTFLS | LVSKLDS | MQGTHFPLT |
| | (SEQ ID NO: 831) | (SEQ ID NO: 846) | (SEQ ID NO: 856) |
| 7F8 | SASSSVSYMY | LTSILAS | QQWSFNPYT |
| | (SEQ ID NO: 833) | (SEQ ID NO: 847) | (SEQ ID NO: 858) |
| 8F8 | RSSQSLVHSNGNTYLH | KVSNRFS | SQSTHVPLT |
| | (SEQ ID NO: 834) | (SEQ ID NO: 848) | (SEQ ID NO: 859) |
| H7 | SASSSVSYMY | LTSILAS | QQWSFNPYT |
| | (SEQ ID NO: 833) | (SEQ ID NO: 847) | (SEQ ID NO: 858) |
| 2H8 | SASSSVSYMY | LTSILAS | QQWSFNPYT |
| | (SEQ ID NO: 833) | (SEQ ID NO: 847) | (SEQ ID NO: 858) |
| 3A2 | RSSQTIIHSNGNTYLE | KVSNRFS | FQGSHVPYT |
| | (SEQ ID NO: 835) | (SEQ ID NO: 848) | (SEQ ID NO: 860) |
| 3A7 | KSSQSLLYSNGKTFLS | LVSKLDS | MQGTHFPLT |
| | (SEQ ID NO: 831) | (SEQ ID NO: 846) | (SEQ ID NO: 856) |
| 3B10 | KSSQSLLYSSDQKNYLA | WASTRES | QQYYSYPLT |
| | (SEQ ID NO: 836) | (SEQ ID NO: 849) | (SEQ ID NO: 855) |
| 4F11 | RSSQTIIHSNGNTYLE | KVSNRFS | FQGSHVPYT |
| | (SEQ ID NO: 835) | (SEQ ID NO: 848) | (SEQ ID NO: 860) |
| 6H6 | KSSQSVFYSSNQKNYLA | WASTRES | HQYLSSLT |
| | (SEQ ID NO: 1401) | (SEQ ID NO: 849) | (SEQ ID NO: 1402) |
| 7A9 | RASENIYSYLA | KAKTLAE | QHHYGTPFT |
| | (SEQ ID NO: 837) | (SEQ ID NO: 850) | (SEQ ID NO: 861) |
| 8A1 | RTSENVYSNLA | AATNLAD | HHFWGTPYT |
| | (SEQ ID NO: 838) | (SEQ ID NO: 851) | (SEQ ID NO: 862) |
| 9F5 | RSSQSLVHSNGYTYLH | KVSNRFS | SQSTRVPYT |
| | (SEQ ID NO: 839) | (SEQ ID NO: 848) | (SEQ ID NO: 863) |
| 9G1 | RFSQSLVHSNGNTYLH | KVSNRFS | SQSTRVPPT |
| | (SEQ ID NO: 840) | (SEQ ID NO: 848) | (SEQ ID NO: 864) |
| 9G3 | KASSNVNYMS | FTSNLPS | SGEVTQFT |
| | (SEQ ID NO: 841) | (SEQ ID NO: 852) | (SEQ ID NO: 865) |
| 10A9 | RSSQTIIHSNGNTYLE | KVSNRFC | FQGSHVPYT |
| | (SEQ ID NO: 835) | (SEQ ID NO: 853) | (SEQ ID NO: 860) |
| 11A8 | KSSQSLLNSGNQKKYLT | WASTRES | QNDYGFPLT |
| | (SEQ ID NO: 842) | (SEQ ID NO: 849) | (SEQ ID NO: 866) |
| 12D9 | KSSQSLLYSGNQKNFLA | WASTRES | QQYYSYPFT |
| | (SEQ ID NO: 843) | (SEQ ID NO: 849) | (SEQ ID NO: 867) |
| 12F9 | KSSQSLLYSSDQKNYLA | WASTRES | QQYYSYPLT |
| | (SEQ ID NO: 836) | (SEQ ID NO: 849) | (SEQ ID NO: 855) |
| 10C1 | KSSQSVFYSSNQKNYLA | WASTRES | HQYLSSLT |
| | (SEQ ID NO: 1401 | (SEQ ID NO: 849) | (SEQ ID NO: 1402) |
| 7E9 | KSSQSLLYSSNQKNCLA | WASTRES | QQYYSYPLT |
| | (SEQ ID NO: 830) | (SEQ ID NO: 849) | (SEQ ID NO: 855) |
| 8C3 | RS SQSLVHSNGNTYLH | KVSNRFS | SQSTHVPPT |
| | (SEQ ID NO: 834) | (SEQ ID NO: 848) | (SEQ ID NO: 1403) |
| IB4v1 | SQDVSTTVA | SASYRYT | QQHYSTPPT |
| | (SEQ ID NO: 1510) | (SEQ ID NO: 1515) | (SEQ ID NO: 1518) |
| IB4v2 | SQSLVHSNGNTYLH | KVSNRVS | SQSTHVPLT |
| | (SEQ ID NO: 1554) | (SEQ ID NO: 1559) | (SEQ ID NO: 859) |
| 6H2 | SQSIVHSNGNTYLE | KVSNRFS | FQGSHVPFT |
| | (SEQ ID NO: 1511) | (SEQ ID NO: 848) | (SEQ ID NO: 1519) |
| 7B11 | SQGVSTAVA | WASTRHT | HQHYSTYT |
| | (SEQ ID NO: 1512) | (SEQ ID NO: 1516) | (SEQ ID NO: 1520) |
| 18D8 | SQDVRTAVA | SASYRYT | QQHYGTPPWT |
| | (SEQ ID NO: 1513) | (SEQ ID NO: 1515) | (SEQ ID NO: 1521) |
| 18E4v1 | SENVVTYVS | GASNRYT | GQGYSYPYT |
| | (SEQ ID NO: 1514) | (SEQ ID NO: 1517) | (SEQ ID NO: 1522) |
| 18E4v2 | SQSLVHSNGNTYLH | KVSDRFS | SQSTHVPLT |
| | (SEQ ID NO: 1554) | (SEQ ID NO: 1560) | (SEQ ID NO: 859) |
| 29F6v1 | SQDVRTAVA | SASYRYT | QQHYGTPPWT |
| | (SEQ ID NO: 1513) | (SEQ ID NO: 1515) | (SEQ ID NO: 1521) |
| 29F6v2 | SQSLVHSNGDTYLH | KVSNRFS | SQSTHVPLT |
| | (SEQ ID NO: 1555) | (SEQ ID NO: 848) | (SEQ ID NO: 859) |
| 40D5 | SQDVRTAVA | SASYRYT | QQHYGTPPWT |
| | (SEQ ID NO: 1513) | (SEQ ID NO: 1515) | (SEQ ID NO: 1521) |
| 43B9 | SQDVRTAVA | SASYRYT | QQHYGTPPWT |
| | (SEQ ID NO: 1513) | (SEQ ID NO: 1515) | (SEQ ID NO: 1521) |
| 44A8v1 | SQDVSTTVA | SASYRYT | QQHYSTPPT |
| | (SEQ ID NO: 1510) | (SEQ ID NO: 1515) | (SEQ ID NO: 1518) |
| 44A8v2 | SESVDYHGTSLMQ | AASNVES | QQNRKILWT |
| | (SEQ ID NO: 1556) | (SEQ ID NO: 1561) | (SEQ ID NO: 1564) |
| 44B4v1 | SQDVRTAVA | SASYRYT | QQHYGTPPWT |
| | (SEQ ID NO: 1513) | (SEQ ID NO: 1515) | (SEQ ID NO: 1521) |
| 44B4v2 | SENIZYSLA | NANSLED | KQAYDVPWT |
| | (SEQ ID NO: 1557) | (SEQ ID NO: 1562) | (SEQ ID NO: 1565) |
| 29F7 | RASQSIGTSIH | FASESIS | QQTNTWPIT |
| | (SEQ ID NO: 1558) | (SEQ ID NO: 1563) | (SEQ ID NO: 1566) |
| 32G1 | RSSQSLVHSNGNTYLH | KVSNRFS | SQSTHVPLT |
| | (SEQ ID NO: 834) | (SEQ ID NO: 848) | (SEQ ID NO: 859) |

TABLE D2
EU or Kabat light chain HVR consensus sequences

| | HVR 1,1 |
|---|---|
| Consensus 1 | RXSENXYSXLA (SEQ ID NO: 1646) |
| Consensus 2 | RSSQXXXHSNGXTYLX (SEQ ID NO: 1647) |
| Consensus 3 | KSSQSXXXSXXQKXXLX (SEQ ID NO: 1648) |

TABLE D3
EU or Kabat heavy chain HVR sequences

| Ab ID | HVR H1 | HVR H2 | HVR H3 |
|---|---|---|---|
| 4D11 | FTLSSYAMS | VASISRGGSTYYP | TRGYGYYRTPFAN |
| | (SEQ ID NO: 868) | (SEQ ID NO: 886) | (SEQ ID NO: 905) |
| 78C5 | FTLSSYAMS | VASISRGGSTYYP | TRGYGYYRTPFAN |
| | (SEQ ID NO: 868) | (SEQ ID NO: 886) | (SEQ ID NO: 905) |
| 6G12 | YTFTEYTMH | IGGINPNNGGTSYS | ARGGSHYYAMDY |
| | (SEQ ID NO: 869) | (SEQ ID NO: 887) | (SEQ ID NO: 906) |
| 8E10 | YTFTDYEMH | IGVIDPETGGTAYN | TSPDYYGSSYPLYYAMDY |
| | (SEQ ID NO: 870) | (SEQ ID NO: 888) | (SEQ ID NO: 907) |
| 7E5 | FTFSDAWMG | VAEIRDKVKNHATYYA | RLGVFDY (SEQ ID NO: 908) |
| | (SEQ ID NO: 871) | (SEQ ID NO: 889) | |
| 7F8 | FSFNTYAMN | IARIRSKSNNYATYYA | VRHGDGNLWYIDV |
| | (SEQ ID NO: 872) | (SEQ ID NO: 890) | (SEQ ID NO: 909) |
| 8F8 | YTVSRYWMH | IGRIDPNSGGTKYN | VLTGTDFDY |
| | (SEQ ID NO: 873) | (SEQ ID NO: 891) | (SEQ ID NO: 910) |
| 1H7 | FSFNTYAMN | IARIRSKSNNYATYY | VRHGDGNLWYIDV |
| | (SEQ ID NO: 872) | A (SEQ ID NO: 890) | (SEQ ID NO: 909) |
| 2H8 | FSFNTYAMN | IARIRSKSNNYATYY | VRHGDGNLWYIDV |
| | (SEQ ID NO: 872) | A (SEQ ID NO: 890) | (SEQ ID NO: 909) |
| 3A2 | YPFSNFWIT | IGDIYPGSDNSNYN | AREAYYTNPGFAY |
| | (SEQ ID NO: 874) | (SEQ ID NO: 892) | (SEQ ID NO: 911) |
| 3A7 | FTFSDAWMG | VAEIRDKVKNHATYYA | RLGVFDY (SEQ ID NO: 908) |
| | (SEQ ID NO: 871) | (SEQ ID NO: 889) | |
| 3B10 | LTSNTYTQT | ESVIRSKSNNFSTLYA | VRHKSNRYPGVY |
| | (SEQ ID NO: 875) | (SEQ ID NO: 893) | (SEQ ID NO: 912) |
| 4F11 | YPFSNFWIT | IGDIYPGSDNSNYN | AREAYYTNPGFAY |
| | (SEQ ID NO: 874) | (SEQ ID NO: 892) | (SEQ ID NO: 911) |
| 6H6 | FTFSDAWMD | VAEIRNKVNNHATYYA | TSLYDGYYLRFAY |
| | (SEQ ID NO: 876) | (SEQ ID NO: 894) | (SEQ ID NO: 913) |
| 7A9 | FTFNTYSMN | VAHIKTKZNNFATFYA | VZHZSNNYPFAY |
| | (SEQ ID NO: 877) | (SEQ ID NO: 895) | (SEQ ID NO: 914) |
| 7B3 | YTFTTYWIH | IGRNDPNSGGSNYN | VRTNWDGDF |
| | (SEQ ID NO: 878) | (SEQ ID NO: 896) | (SEQ ID NO: 915) |
| 8A1 | YAFSNYWMS | IGQIYPGDGDTKYN | SREKGADYYGSTYSAWFSY |
| | (SEQ ID NO: 879) | (SEQ ID NO: 897) | (SEQ ID NO: 916) |
| 9F5 | YAFSSSWMN | IGRIYPGDGDTNYN | ARLLRNQPGESYAMDY |
| | (SEQ ID NO: 880) | (SEQ ID NO: 898) | (SEQ ID NO: 917) |
| 9F5a | YAFSSSWMN | RIYPGDGDTNYNGEFRV | ARLLRNQPGESYAMDY |
| | (SEQ ID NO: 880) | (SEQ ID NO: 1708) | (SEQ ID NO: 917) |
| 9G1 | YIFTTYWIH | IGRIDPNNGDTNYN | VMTGTDFDY |
| | (SEQ ID NO: 881) | (SEQ ID NO: 899) | (SEQ ID NO: 918) |
| 9G3 | FNFNTYAMK | IARIRSNSNDYATNYS | VGHKINNYPFAH |
| | (SEQ ID NO: 882) | (SEQ ID NO: 900) | (SEQ ID NO: 919) |
| 10A9 | YPFSNFWIT | IGDIYPGSDNRNFN | AREAYYTNPGFAY |
| | (SEQ ID NO: 874) | (SEQ ID NO: 901) | (SEQ ID NO: 911) |
| 11A8 | FNFNTYAMN | VARIRSKSNNYATYYA | VRHYSNYGWGFAY |
| | (SEQ ID NO: 883) | (SEQ ID NO: 902) | (SEQ ID NO: 920) |
| 12D9 | YTFSDYYIH | IGYIYPNNGDNGYN | ARRGYYGGSYDY |
| | (SEQ ID NO: 884) | (SEQ ID NO: 903) | (SEQ ID NO: 921) |
| 12F9 | FRFNTYAMT | EGVIRRKSSNFATLYA | VRHKSNKYPFVY |
| | (SEQ ID NO: 885) | (SEQ ID NO: 904) | (SEQ ID NO: 922) |
| 10C1 | FTFSDAWMD | VAEIRNKINNHATYYA | TSLYDGSYLRFAY |
| | (SEQ ID NO: 876) | (SEQ ID NO: 1405) | (SEQ ID NO: 1408) |
| 7E9 | YTFTEYTMH | IGGINPNNGGTSYK | ARGGSHYYAMDY |
| | (SEQ ID NO: 869) | (SEQ ID NO: 1406) | (SEQ ID NO: 906) |
| 8C3 | YSFTGYYMH | IGRVNPNNGGTSYN | VLTGGYFDY |
| | (SEQ ID NO: 1404) | (SEQ ID NO: 1407) | (SEQ ID NO: 1409) |
| 1B4 | SRFTFSSYAMS | VAAISGGGRYTYYP | ARHYDGYLDY |
| | (SEQ ID NO: 1523) | (SEQ ID NO: 1526) | (SEQ ID NO: 1529) |
| 6H2 | SAFSLTNYAVH | LGVIWSGGSTAFN | ATHYYRSTYAFSY |
| | (SEQ ID NO: 1524) | (SEQ ID NO: 1527) | (SEQ ID NO: 1530) |
| 7B11v1 | SRFTFSSYAMS | VAAISGGGRYTYYP | ARHYDGYLDY |
| | (SEQ ID NO: 1523) | (SEQ ID NO: 1526) | (SEQ ID NO: 1529) |
| 7B11v2 | SGYTFTDFYMN | IGDINPNNGHTTYN | AREPYSYGSSPWYFLV |
| | (SEQ ID NO: 1567) | (SEQ ID NO: 1575) | (SEQ ID NO: 1583) |
| 18D8 | SRFTFSSYAMS | VAAISGGGRYTYYP | ARHYDGYLDY |
| | (SEQ ID NO: 1523) | (SEQ ID NO: 1526) | (SEQ ID NO: 1529) |
| 18E4v1 | SRFTFSSYAVS | VATISGGGRYTYYP | ARHYDGYLDY |
| | (SEQ ID NO: 1525) | (SEQ ID NO: 1528) | (SEQ ID NO: 1529) |
| 18E4v2 | SGYTFTAYWMH | IGRTHPSDSDTNYN | ATYSNYVTGAMDS |
| | (SEQ ID NO: 1568) | (SEQ ID NO: 1576) | (SEQ ID NO: 1584) |
| 29F6v1 | SRFTFSSYAMS | VAAISGGGRYTYYP | ARHYDGYLDY |
| | (SEQ ID NO: 1523) | (SEQ ID NO: 1526) | (SEQ ID NO: 1529) |
| 29F6v2 | SGFNIKNTYIH | IGRIDPAIGNTNYA | VSPGMDY |
| | (SEQ ID NO: 1569) | (SEQ ID NO: 1577) | (SEQ ID NO: 1585) |
| 40D5v1 | SRFTFSSYAMS | VAAISGGGRYTYYP | ARHYDGYLDY |
| | (SEQ ID NO: 1523) | (SEQ ID NO: 1526) | (SEQ ID NO: 1529) |
| 40D5v2 | SGYTFTNYWIH | IGRIHPSDSDINYN | VKTGTSFAS |
| | (SEQ ID NO: 1570) | (SEQ ID NO: 1578) | (SEQ ID NO: 1586) |
| 43B9 | SRFTFSSYAMS | VAAISGGGRYTYYP | ARHYDGYLDY |
| | (SEQ ID NO: 1523) | (SEQ ID NO: 1526) | (SEQ ID NO: 1529) |
| 44A8 | SRFTFSSYAMS | VAAISGGGRYTYYP | ARHYDGYLDY |
| | (SEQ ID NO: 1523) | (SEQ ID NO: 1526) | (SEQ ID NO: 1529) |
| 44B4v1 | SRFTFSSYAMS | VAAISGGGRYTYYP | ARHYDGYLDY |
| | (SEQ ID NO: 1523) | (SEQ ID NO: 1526) | (SEQ ID NO: 1529) |
| 44B4v2 | SGYTFTSATMH | IGYINPNSGYSKYN | ARWGIDGNYGGGFFDV |
| | (SEQ ID NO: 1571) | (SEQ ID NO: 1579) | (SEQ ID NO: 1587) |
| 45D6 | YSFTDYNIH | IGYINPNSDNTRYI | TRGFSNLGAMDY |
| | (SEQ ID NO: 1572) | (SEQ ID NO: 1580) | (SEQ ID NO: 1588) |
| 29F7 | FTLSNYWMN | VAQIRLKSDNYATHYA | TGAGGNHENY |
| | (SEQ ID NO: 1573) | (SEQ ID NO: 1581 | (SEQ ID NO: 1589) |
| 32G1 | YTFTDYNIH | IGYINPNNGGTTYN | ATTYVSFSY |
| | (SEQ ID NO: 1574) | (SEQ ID NO: 1582 | (SEQ ID NO: 1590) |

TABLE D4
EU or Kabat heavy chain HVR consensus sequences

| | HVR H1 | HVR H2 |
|---|---|---|
| Consensus 1 | YX₁X₂X₃XYXXH | IGXXXPX₁X₂X₃X₄X₅XYX₆ |
| | X₁ is T or S | X₁ is N or E |
| | X₂ is F or V | X₂ is N, S, or T |
| | X₃ is T or S | X₃ is G or D |
| | (SEQ ID NO: 1649) | X₄ is G, D, or N |
| | | X₅ is T, S, or N |
| | | X₆ is N, 5, K, or I |
| | | (SEQ ID NO: 1656) |
| Consensus 2 | YTFTXYXXH | IGXXXPNNGGTXYN (SEQ ID NO: 1657) |
| | (SEQ ID NO: 1650) | |
| Consensus 3 | FTFSDAWMX₁ | VAEIRX₁KX₂X₃NHATYYA |
| | X₁ is D or G (SEQ ID NO: 1651) | X₁ is N or D |
| | | X₂ iS V or I |
| | | X₃ is N or K (SEQ ID NO: 1658) |
| Consensus 4 | FXX₁X₂X₃YX₄MX₅ | XX₁XIX₂X₃X₄X₅X₆X₇X₈ATXYX₉ |
| | x₁ is F or L | X₁ is A or G |
| | X₂ is N or S | X₂ is R or K |
| | X₃ is T or N | X₃ is S, T, R, or L |
| | X₄ is A, S, or W | X₄ is K or N |
| | X₅ is N, K, or T | X₅ is S, E, or Q |
| | (SEQ ID NO: 1652) | X₆ is N, S, or D |
| | | X₇ is N or D |
| | | X₈ is Y or F |
| | | X₉ is A or S (SEQ ID NO: 1659) |
| Consensus 5 | FXFNTYAMN | XAXIRSKSNNYATXYA |
| | (SEQ ID NO: 1653) | (SEQ ID NO: 1660) |
| Consensus 6 | YXFX₁X₂XWX₃X | IGXIX₁PX₂XX₃X₄X₅X₆X₇N |
| | X₁ is S or T | X₁ is Y or D |
| | X₂ is N, S, or T | X₂ is G or N |
| | X₃ iS I or M (SEQ ID NO: 1654) | X₃ is G or D |
| | | X₄ is N or D |
| | | X₅ is T, R, or S |
| | | X₆ is N or K |
| | | X₇ is Y or F (SEQ ID NO: 1661) |
| Consensus 7 | YXFSNMVIX | IGXIYPGXGDTNYN (SEQ ID NO: 1662) |
| | (SEQ ID NO: 1655) | |

TABLE D5
EU or Kabat light chain Framework sequences

| Ab ID | VL FR1 | VL FR2 | VL FR3 | VL FR4 |
|---|---|---|---|---|
| 4D11 | DIZVTQSPASLSAS | WYQLKQGKSPQ | GVPSRFSGSGSGTQFS | FGGGTKLEIK |
| | VGETVTITC | LLVY | LRINSLQPEDFGSYYC | (SEQ ID NO: 968) |
| | (SEQ ID NO: 923) | (SEQ ID NO: 940) | (SEQ ID NO: 950) | |
| 78C5 | DIZVTQSPASLSAS | WYQLKQGKSPQ | GVPSRFSGSGSGTQFS | FGGGTKLEIK |
| | VGETVTITC | LLVY | LRINSLQPEDFGSYYC | (SEQ ID NO: 968) |
| | (SEQ ID NO: 923) | (SEQ ID NO: 940) | (SEQ ID NO: 950) | |
| 6G12 | TMSQSPSSLAVSV | WYQQKPGQSPK | GVPDRFTGSGSGTDF | FGAGTKLELK |
| | GEKVTMSC | LLIY | TLTISSVKAEDLAVY | (SEQ ID NO: 969) |
| | (SEQ ID NO: 924) | (SEQ ID NO: 941) | YC (SEQ ID NO: 951) | |
| 8F11 | DVZMTQTPLTLSV | WLLQRPGQSPK | GVPDRFAGSGSGTDF | FGAGTKLELK |
| | TIGQPASISC | RLIY | TLKISRLEADDLGIYY | (SEQ ID NO: 969) |
| | (SEQ ID NO: 925) | (SEQ ID NO: 942) | C (SEQ ID NO: 952) | |
| 8E10 | DVZMTQTPLTLSV | WLLQRPGQSPK | GVPDRFTGSGSGTDF | FGGGTKLEIK |
| | TIGQPASISC | RLIY | TLKISRVEAEDLGVY | (SEQ ID NO: 968) |
| | (SEQ ID NO: 925) | (SEQ ID NO: 942) | YC (SEQ ID NO: 953) | |
| 7E5 | DVZMTQTPLTLSV | WLLQRPGQSPK | GVPDRFAGSGSGTDF | FGAGTKLELK |
| | TIGQPASISC | RLIY | TLKISRLEADDLGIYY | (SEQ ID NO: 969) |
| | (SEQ ID NO: 925) | (SEQ ID NO: 942) | C (SEQ ID NO: 952) | |
| 7E5v2 | DVVMTQTPLTLSV | WLLQRPGQSPK | GVPDRFAGSGSGTDF | FGAGTKLELK |
| | TIGQPASISC | RLIY | TLKISRLEADDLGIYY | (SEQ ID NO: 969) |
| | (SEQ ID NO: 931) | (SEQ ID NO: 942) | C (SEQ ID NO: 952) | |
| 7F8 | VLTQSPALMSASP | WYQQKPRSSPK | GVPARFSGSGSGTSY | FGGGTKLVIK |
| | GEKVTMTC | PWIY | SLTINNMEAEDAATY | (SEQ ID NO: 970) |
| | (SEQ ID NO: 926) | (SEQ ID NO: 943) | YC (SEQ ID NO: 954) | |
| 8F8 | DVZMTQTPLSLPV | WYLQKPGQSPK | GVPDRFSGSGSGTDF | FGAGTKLELK |
| | SLGDQASISC | LLIY | TLKISRVEAEDLGVY | (SEQ ID NO: 969) |
| | (SEQ ID NO: 927) | (SEQ ID NO: 944) | FC (SEQ ID NO: 955) | |
| 1H7 | VLTQSPAIMZASP | WYQQKPRSSPK | GVPARFSGSGSGTSY | FGGGTKLVIK |
| | GEKVTMTC | PWIY | SLTISSMEAEDAATY | (SEQ ID NO: 970) |
| | (SEQ ID NO: 928) | (SEQ ID NO: 943) | YC (SEQ ID NO: 956) | |
| 2H8 | NVLTQSPALMSAS | WYQQKPRSSPK | GVPARFSGSGSGTSY | FGGGTKLVIK |
| | PGEKVTMTC | PWIY | SLTISSMEAEDAATY | (SEQ ID NO: 970) |
| | (SEQ ID NO: 929) | (SEQ ID NO: 943) | YC (SEQ ID NO: 956) | |
| 3A2 | DVVMTQTPLSLPV | WYLRKPGQSPK | GVPDRFSGSGSGTDF | FGGGTELEIK |
| | SLGDQASISC | LLIY | TLKISRVEAEDLGVY | (SEQ ID NO: 971) |
| | (SEQ ID NO: 930) | (SEQ ID NO: 945) | YC (SEQ ID NO: 957) | |
| 3A7 | DVVMTQTPLTLSV | WLLQRPGQSPK | GVPDRFAGSGSGTDF | FGGGTKLEMK |
| | TIGQPASISC | RLIY | TZKISRLEADDLGIYY | (SEQ ID NO: 972) |
| | (SEQ ID NO: 931) | (SEQ ID NO: 942) | C (SEQ ID NO: 958) | |
| 3B10 | ITMSQSPSSLAVSV | WYQQKPGQSPK | GVPDRFTGSGSGTDF | FGAGTKLELK |
| | GEKVTMSC | LLIY | TLTISSVKAEDLAVY | (SEQ ID NO: 969) |
| | (SEQ ID NO: 932) | (SEQ ID NO: 941) | CC (SEQ ID NO: 959) | |
| 4F11 | DVZMTQTPLSLPV | WYLRKPGQSPK | GVPDRFSGSGSGTDF | FGGGTELEIK |
| | SLGDQASISC | LLIY | TLKISRVEGEDLGVY | (SEQ ID NO: 971) |
| | (SEQ ID NO: 927) | (SEQ ID NO: 945) | YC (SEQ ID NO: 960) | |
| 6H6 | QTQSPSSLAVSAG | WYQQKPGQSPK | GVPDRFTGSGFGTDF | FGAGTKLELK |
| | EKVTLSC | LLIS | TLTISSVQGEDLAVY | (SEQ ID NO: 969) |
| | (SEQ ID NO: 1410) | (SEQ ID NO: 1413) | YC (SEQ ID NO: 1414) | |
| 7A9 | QMSQSPACLZAZV | WYQQKQGKSPK | GVPSRFSGRGSGTQF | FGSGTKLEIK |
| | GESVTITC | LVVY | FLKINSZQREDFGSY | (SEQ ID NO: 973) |
| | (SEQ ID NO: 933) | (SEQ ID NO: 946) | YC (SEQ ID NO: 961) | |
| 8A1 | DIQMTQSPASLSVS | WYQQKQGKSPQ | GVPSRFSASGSATQFS | FGGGTKLEMN |
| | VGETVTITC | LLVY | LKINSLQSADFGSYY | (SEQ ID NO: 974) |
| | (SEQ ID NO: 934) | (SEQ ID NO: 947) | C (SEQ ID NO: 962) | |
| 9F5 | DVZMTQNPLSLPV | WYLQKPGQSPK | GVPDRFSGSGSGTDF | FGGGTKLEIK |
| | SLGDQASISC | LLIY | TLKISRVEADDLGVY | (SEQ ID NO: 968) |
| | (SEQ ID NO: 935) | (SEQ ID NO: 944) | LC (SEQ ID NO: 963) | |
| 9F5v2 | DVVMTQTPLSLPV | WYLQKPGQSPK | GVPDRFSGSGSGTDF | FGGGTKLEIK |
| | SLGDQASISC | LLIY | TLKISRVEADDLGVY | (SEQ ID NO: 968) |
| | (SEQ ID NO: 930) | (SEQ ID NO: 944) | FC (SEQ ID NO: 1668) | |
| 9G1 | DVLMTQTPLSLPV | WYLQKPGQSPK | GVPDRFSGSGSGTDF | FGGGTKLEIK |
| | SLGDQASISC | LLIY | TLRISGVEAEDLGVY | (SEQ ID NO: 968) |
| | (SEQ ID NO: 936) | (SEQ ID NO: 944) | FC (SEQ ID NO: 964) | |
| 9G3 | NVLTQSPALIWAZ | WXXXKPRSSPK | GVPGRFSGSGSGTYX | FGGGTKLEMK |
| | PGEKVTMTC | PGIY | SFKISSMEGKMGPLII | (SEQ ID NO: 975) |
| | (SEQ ID NO: 937) | (SEQ ID NO: 948) | FC (SEQ ID NO: 965) | |
| 10A9 | DVVMTQTPLSLPV | WYLRKPGQSPK | GVPDRFSGSGSGTDF | FGGGTELEIK |
| | SLGDQASISC | LLIY | TLKISRVEAEDLGVY | (SEQ ID NO: 971) |
| | (SEQ ID NO: 930) | (SEQ ID NO: 945) | YC (SEQ ID NO: 957) | |
| 11A8 | DIZMTQSPSSLTVT | WYQQKPGQPZK | GVRDRFTGSGZGTDF | FGGGTKLEMK |
| | AGEKVTMSC | LLIY | TLTISSVQGEDLAIYY | (SEQ ID NO: 972) |
| | (SEQ ID NO: 938) | (SEQ ID NO: 949) | C (SEQ ID NO: 966) | |
| 12D9 | TQSPSSLAVSVGE | WYQQKPGQSPK | GVPDRFTGSGSGTDF | FGSGTKLEIK |
| | KVTMTC | LLIY | TLTISTVKAEDLAVY | (SEQ ID NO: 973) |
| | (SEQ ID NO: 939) | (SEQ ID NO: 941) | YC (SEQ ID NO: 967) | |
| 12F9 | TMSQSPSSLAVSV | WYQQKPGQSPK | GVPDRFTGSGSGTDF | FGAGTKLELK |
| | GEKVTMSC | LLIY | TLTISSVKAEDLAVY | (SEQ ID NO: 969) |
| | (SEQ ID NO: 924) | (SEQ ID NO: 941) | CC (SEQ ID NO: 959) | |
| 10C1 | QTQVFLSLLLWVS | WYQQKPGQSPK | GVPDRFTGSGSGTDF | FGAGTKLELK |
| | GTCGNIMLTQSPSS | LLIS | TLTINSVQAEDLAVY | (SEQ ID NO: 969) |
| | LAVSAGEKVTLSC | (SEQ ID NO: 1413) | YC (SEQ ID NO: 1415) | |
| | (SEQ ID NO: 1411) | | | |
| 7E9 | DIVMSQSPSSLAVS | WYQQKPGQSPK | GVPDRFTGSGSGTDF | FGAGTKLELK |
| | VGEKVTMSC | LLIY | TLTISSVKAEDLAVY | (SEQ ID NO: 969) |
| | (SEQ ID NO: 1412) | (SEQ ID NO: 941) | YC (SEQ ID NO: 951) | |
| 8C3 | DVVMTQTPLSLPV | WYLQKPGQSPK | GVPDRFSGSGSGTDF | FGSGTKLEIK |
| | SLGDQASISC | LLIY | TLKISRVEAEDLGVY | (SEQ ID NO: 973) |
| | (SEQ ID NO: 930) | (SEQ ID NO: 944) | FC (SEQ ID NO: 955) | |
| IB4v1 | DIVMTQSHKFMST | WYQQKPGQSPK | GVPDRFTGSGFGTDF | FGGGTKLEIK |
| | SVGDRVSITCKA | LLIY | TFTISSVQAEDLAVY | (SEQ ID NO: 968) |
| | (SEQ ID NO: 1531) | (SEQ ID NO: 941) | YC (SEQ ID NO: 1535) | |
| IB4v2 | ZVVZTQTPLSLPVS | WFLQKPGQSPK | GVPDRFSGSGSGTDF | FGAGTKLELK |
| | LGDQASFSCRS | LLIF | TLKISRVEAEDLGVY | (SEQ ID NO: 969) |
| | (SEQ ID NO: 1591) | (SEQ ID NO: 1597) | FC (SEQ ID NO: 955) | |
| 6H2 | DVLMTQTPLSLPV | WYLQKPGQSPK | GVPDRFSGSGSGTDF | FGSGTKLEIK |
| | SLGDQASISCRS | LLIY | TLKISRVEAEDLGVY | (SEQ ID NO: 973) |
| | (SEQ ID NO: 1532) | (SEQ ID NO: 944) | YC (SEQ ID NO: 957) | |
| 7B11 | DIVMTQSHKFMST | WYQQKPGQSPK | GVPDRFTGSGSGTDY | FGGGTKLEIK |
| | SVGDRVSITCKA | LLIY | TLTISSVQAEDLALY | (SEQ ID NO: 968) |
| | (SEQ ID NO: 1531) | (SEQ ID NO: 941) | YC (SEQ ID NO: 1536) | |
| 18D8 | DIVMTQSHKFMST | WYQQKPGQSPK | GVPDRFTGSGFGTDF | FGGGTKLEIK |
| | SIGARVSITCKA | LLIY | TFTISSVQAEDLAVY | (SEQ ID NO: 968) |
| | (SEQ ID NO: 1533) | (SEQ ID NO: 941) | YC (SEQ ID NO: 1535) | |
| 18E4v1 | DIVMTQSPKSMSM | WYQQKPGQSPK | GVPDRFTGSGSATDF | FGGGTKLEIK |
| | SVGERVTLTCKA | LLIY | TLTISSVQAEDLADY | (SEQ ID NO: 968) |
| | (SEQ ID NO: 1534) | (SEQ ID NO: 941) | HC (SEQ ID NO: 1537) | |
| 18E4v2 | NIVMTQSPKSMSM | WYQQKPGQSPK | GVPDRFTGSGSATDF | FGGGTKLEIK |
| | SVGERVTLTCKA | LLIY | TLTISSVQAEDLADY | (SEQ ID NO: 968) |
| | (SEQ ID NO: 1592) | (SEQ ID NO: 941) | HC (SEQ ID NO: 1537) | |
| 18E4v3 | DVVMTQTPLSLPV | WYLQKPGQSPK | GVPDRFSGSGSGTDF | FGAGTKLELK |
| | SLGDQASISCRS | LLIY | TLRISRVEAEDLGVY | (SEQ ID NO: 969) |
| | (SEQ ID NO: 1593) | (SEQ ID NO: 944) | FC (SEQ ID NO: 1601) | |
| 29F6v1 | DIVMTQSHKFMST | WYQQKPGQSPK | GVPDRFTGSGSGTDF | FGGGTKLEIK |
| | SIGARVSITCKA | LLIY | TFTISSVQAEDLAVY | (SEQ ID NO: 968) |
| | (SEQ ID NO: 1533) | (SEQ ID NO: 941) | YC (SEQ ID NO: 1538) | |
| 29F6v2 | DVVMTQTPLSLPV | WYLQKPGQSPK | GVPDRFSGSGSGTDF | FGAGTKLELK |
| | SLGDQASISCRS | LLIY | TLKISRVEAEDLGVY | (SEQ ID NO: 969) |
| | (SEQ ID NO: 1593) | (SEQ ID NO: 944) | FC (SEQ ID NO: 955) | |
| 40D5 | DIVMTQSHKFMST | WYQQKPGQSPK | GVPDRFTGSGSGTDF | FGGGTKLEIK |
| | SIGARVSITCKA | LLIY | TFTISSVQAEDLAVY | (SEQ ID NO: 968) |
| | (SEQ ID NO: 1533) | (SEQ ID NO: 941) | YC (SEQ ID NO: 1538) | |
| 43B9 | DIVMTQSHKFMST | WYQQKPGQSPK | GVPDRFTGSGSGTDF | FGGGTKLEIK |
| | SIGARVSITCKA | LLIY | TFTISSVQAEDLAVY | (SEQ ID NO: 968) |
| | (SEQ ID NO: 1533) | (SEQ ID NO: 941) | YC (SEQ ID NO: 1538) | |
| 44A8v1 | DIVMTQSHKFMST | WYQQKPGQSPK | GVPDRFTGSGSGTDF | FGGGTKLEIK |
| | SVGDRVSITCKA | LLIY | TFTISSVQAEDLAVY | (SEQ ID NO: 968) |
| | (SEQ ID NO: 1531) | (SEQ ID NO: 941) | YC (SEQ ID NO: 1538) | |
| 44A8v2 | DIVLTQSPASLAVS | WYQQKPGQPPK | GVPARFSGSGSGTDF | FGGGTKLEIK |
| | LGQRATISCRA | LLIY | SLNIHPVEEDDIAMY | (SEQ ID NO: 968) |
| | (SEQ ID NO: 1594) | (SEQ ID NO: 1598) | FC (SEQ ID NO: 1602) | |
| 44B4v1 | DIVMTQSHKFMST | WYQQKPGQSPK | GVPDRFTGSGSGTDF | FGGGTKLEIK |
| | SIGARVSITCKA | LLIY | TFTISSVQAEDLAVY | (SEQ ID NO: 968) |
| | (SEQ ID NO: 1533) | (SEQ ID NO: 941) | YC (SEQ ID NO: 1538) | |
| 44B4v2 | DIQMTQFPASLAA | WYQQKQGKSPQ | GVPSRFSGSGSGTQY | FGGGTKLEIK |
| | XVGESVTITCRA | LLIY | SMKINSMQPEDTAIY | (SEQ ID NO: 968) |
| | (SEQ ID NO: 1595) | (SEQ ID NO: 1599) | FC (SEQ ID NO: 1603) | |
| 29F7 | ILLTQSPAILSVSPG | WYQQRTNGSPR | GIPSRFSGSGSGTDFT | FGAGTKLELK |
| | ERVSFSC | LLIK | LNINSVESEDIADYYC | (SEQ ID NO: 969) |
| | (SEQ ID NO: 1596) | (SEQ ID NO: 1600) | (SEQ ID NO: 1604) | |
| 32G1 | DVVMTQTPLSLPV | WYLQKPGQSPK | GVPDRFSGSGSGTDF | FGAGTKLELK |
| | SLGDQASISC | LLIY | TLKISRVEAEDLGVY | (SEQ ID NO: 969) |
| | (SEQ ID NO: 930) | (SEQ ID NO: 944) | FC (SEQ ID NO: 955) | |

TABLE D6
EU or Kabat heavy chain Framework sequences

| Ab ID | VH FR1 | VH FR2 | VH FR3 | VH FR4 |
|---|---|---|---|---|
| 44D11 | EVKLVESGGGLV | WVRQTPEKRLEW | DSVQGRFTFSRDNA | WGQGTLVTVSA |
| | KPGGSLKLSCAAS | (SEQ ID NO: 995) | RNILYLQMSSLRSED | (SEQ ID NO: 1029) |
| | G (SEQ ID NO: 976) | | TAMYYC | |
| | | | (SEQ ID NO: 1008) | |
| 78C5 | EVKLVESGGGLV | WVRQTPEKRLEW | DSVQGRFTFSRDNA | WGQGTLVTVSA |
| | KPGGSLKLSCAAS | (SEQ ID NO: 995) | RNILYLQMSSLRSED | (SEQ ID NO: 1029) |
| | G (SEQ ID NO: 976) | | TAMYYC | |
| | | | (SEQ ID NO: 1008) | |
| 6G12 | EVQLQQSGPELVK | WVKQSHGKSLEW | QKFKGKASLTVDKS | WGQGTSVTVSS |
| | PGTSVKISCKTSG | (SEQ ID NO: 996) | SSTAYMELHSLASD | (SEQ ID NO: 1030) |
| | (SEQ ID NO: 977) | | DSAVYYC | |
| | | | (SEQ ID NO: 1009) | |
| 8E10 | QVQLQQSGAELV | WVKQTPVHGLEW | QKFKGKAILTADKS | WGQGTSVTVSS |
| | RPGASVTLSCKAS | (SEQ ID NO: 997) | SSTAYMELRSLTSED | (SEQ ID NO: 1030) |
| | G (SEQ ID NO: 978) | | SAVYYC | |
| | | | (SEQ ID NO: 1010) | |
| 7E5 | EVKLEESGGGLVQP | WVRQSPEKGLEW | ESVKGRFTISRDDSK | WGQGTTLTVSS |
| | GGSMKLSCAASG | (SEQ ID NO: 998) | STVYLQMNTLRADD | (SEQ ID NO: 1031) |
| | (SEQ ID NO: 979) | | TGIYYC | |
| | | | (SEQ ID NO: 1011) | |
| 7F8 | EVQLVESGGGLV | WVRQAPGKGLEW | DSVKDRITCSRDDSE | WGTGTTVTVST |
| | QPKGSLKLSCAA | (SEQ ID NO: 999) | NMFYLQLSSLKTED | (SEQ ID NO: 1032) |
| | SG | | TAMYYC | |
| | (SEQ ID NO: 980) | | (SEQ ID NO: 1012) | |
| 8F8 | QVQLQQSGAELVK | WVKQRPGRGLEW | EKFKTKATLTVDKP | WGQGTTLTVSS |
| | PGASVKLSCKASG | (SEQ ID NO: 1000) | SSTAYMQVSSLTSE | (SEQ ID NO: 1031) |
| | (SEQ ID NO: 981) | | DSAVYYC | |
| | | | (SEQ ID NO: 1013) | |
| IH7 | ZVQLVESGGGLV | WVRQAPGKGLEW | DSVKDRFTCSRDDS | WGTGTTVTVSS |
| | QPKGSLKLSCAA | (SEQ ID NO: 999) | ENMFYLQLSSLKTE | (SEQ ID NO: 1033) |
| | SG | | DTAIYYC | |
| | (SEQ ID NO: 982) | | (SEQ ID NO: 1014) | |
| 2HS | EVQLVESGGGLVQP | WVRQAPGKGLEW | DSVKDRFTCSRDDS | WGTGTTVTVSS |
| | KGSLKLSCAASG | (SEQ ID NO: 999) | ENMFYLQLSSLKTE | (SEQ ID NO: 1033) |
| | (SEQ ID NO: 980) | | DTAMYYC | |
| | | | (SEQ ID NO: 1015) | |
| 3A2 | QVQLQQSGAELVK | WVKQRPGQGLV | EKFKTKATLTVDTSS | WGQGTLVTVST |
| | PGASVKMSCKTSG | W | STAYMHLSSLTSEDS | (SEQ ID NO: 1034) |
| | (SEQ ID NO: 983) | (SEQ ID NO: 1001) | AVYFC | |
| | | | (SEQ ID NO: 1016) | |
| 3A7 | EVKLEESGGGLVQP | WVRQSPEKGLEW | ESVKGRFTISRDDSK | WGQGTTLTVSS |
| | GGSMKLSCAASG | (SEQ ID NO: 998) | STVYLQMNTLRADD | (SEQ ID NO: 1031) |
| | (SEQ ID NO: 979) | | TGIYYC | |
| | | | (SEQ ID NO: 1011) | |
| 3B10 | EVQLVZZGRGZSQ | GVPQGPGKGREW | DSVKDRFTZSRDDS | WGQGTIVTVS |
| | GKGSXZZGRAZRC | (SEQ ID NO: 1002) | ESLFYZQMSZZKZE | (SEQ ID NO: 1035) |
| | (SEQ ID NO: 984) | | DTAMYYZ | |
| | | | (SEQ ID NO: 1017) | |
| 4F11 | QVQLQQSGAELVK | WVKQRPGQGLV | EKFKTKATLTVDTSS | WGQGTLVTVST |
| | PGASVKMSCKTSG | W | STAYMHLS | (SEQ ID NO: 1034) |
| | (SEQ ID NO: 983) | (SEQ ID NO: 1001) | SLTSEDSAVYFC | |
| | | | (SEQ ID NO: 1016) | |
| 6H6 | EVKLEESGGGLVQP | WVRQSPEKGLEW | ESVKGRFTISRDDSK | WGQGTLVTVSA |
| | GGSMKLSCTASG | (SEQ ID NO: 998) | STVYLQMNSLRTED | (SEQ ID NO: 1029) |
| | (SEQ ID NO: 985) | | TGIYYC | |
| | | | (SEQ ID NO: 1018) | |
| 7A9 | LSCAASG | WVRQAPGKGLEW | DSVKDRFTISRDDSE | WGQGTLVTVSA |
| | (SEQ ID NO: 986) | (SEQ ID NO: 999) | SMLYLQMZNLKTED | (SEQ ID NO: 1029) |
| | | | TAMYYC | |
| | | | (SEQ ID NO: 1019) | |
| 7B3 | QVQLQQSGAVLVK | WVKQRPGRGPEW | EKFRNKAILTVDKPS | WGQGTTLTVSS |
| | PGASVKLSCKASG | (SEQ ID NO: 1003) | STAYMQLNSLTSED | (SEQ ID NO: 1031) |
| | (SEQ ID NO: 987) | | ZAVYYC | |
| | | | (SEQ ID NO: 1020) | |
| 8A1 | EVQLQQSGAELVK | WVKQRPGKGLEW | GKFEGKATLTADKS | WGQGTLVTVSA |
| | PGASVKISCKASG | (SEQ ID NO: 1004) | SSTAYMQLSSLTSED | (SEQ ID NO: 1029) |
| | (SEQ ID NO: 988) | | SAVYFC | |
| | | | (SEQ ID NO: 1021) | |
| 9F5 | QVQLQQSGPELVK | WVKQRPGKGLEW | GEFRVRATLTADTSS | WGQGASVTVSS |
| | PGASLKISCKASG | (SEQ ID NO: 1004) | TTAYMQLSSLTSEDS | (SEQ ID NO: 1036) |
| | (SEQ ID NO: 989) | | AVYFC | |
| | | | (SEQ ID NO: 1022) | |
| 9G1 | QVQLQQSGAELVK | WVKQRPGRGPEW | EKFKTKATLTVDKP | WGQGTTLTVSS |
| | PGASVKLSCKASG | (SEQ ID NO: 1003) | SSTADMQLSSLTSED | (SEQ ID NO: 1031) |
| | (SEQ ID NO: 981) | | SAVYYC | |
| | | | (SEQ ID NO: 1023) | |
| 9G3 | EVQLVESGGGLVQP | WVRQTPGKGLEW | DSVKDRFTISRDDSE | WGRGTLV |
| | KGSLKLSCAAFG | (SEQ ID NO: 1005) | SIVYVQMNNLKTED | (SEQ ID NO: 1037) |
| | (SEQ ID NO: 990) | | TGMYSC | |
| | | | (SEQ ID NO: 1024) | |
| 10A9 | QVQLQQSGAEVVK | WVKQRPGQGLV | ERFKTKATLTVDTSS | WGQGTLVTVSA |
| | PGASVKMSCKTSG | W | STAYMHLSSLTSEDS | (SEQ ID NO: 1029) |
| | (SEQ ID NO: 991) | (SEQ ID NO: 1001) | AVYFC | |
| | | | (SEQ ID NO: 1025) | |
| 11A8 | EVQLVESGGRLVQP | WVRQAPGKGLEW | DSVKDRFTISRDDSE | WGQGTLVTVSA |
| | KGSLKLSCAASG | (SEQ ID NO: 999) | SMLYLQMNNLKTE | (SEQ ID NO: 1029) |
| | (SEQ ID NO: 992) | | DTAMYYC | |
| | | | (SEQ ID NO: 1026) | |
| 12D9 | QVQLQQYGPELVK | WMKQSHGKSLE | QEFKGKATLTVDKS | WGQGT |
| | PGASVKMSCKVSG | W | SS | (SEQ ID NO: 1038) |
| | (SEQ ID NO: 993) | (SEQ ID NO: 1006) | TAYMELRSLTFEDS | |
| | | | AV YZC | |
| | | | (SEQ ID NO: 1027) | |
| 12F9 | WRIGQGKGSLKLA | RVRQGPGKGREW | DSVKDRFRASRDDS | WGQGTLVTVSA |
| | RAARG | (SEQ ID NO: 1007) | ES | (SEQ ID NO: 1029) |
| | (SEQ ID NO: 994) | | MLYVQMSNWKQED | |
| | | | T AMYYG | |
| | | | (SEQ ID NO: 1028) | |
| iOCi | GVQSEVKFEESGG | WVRQSPEKGLEW | ESVKGRFTISRDDSK | WGQGTLVTVSA |
| | GLVQPGGSMKLSC | (SEQ ID NO: 998) | SSVSLQMNSLRTED | (SEQ ID NO: 1029) |
| | TASG | | TGIYYC | |
| | (SEQ ID NO: 1416) | | (SEQ ID NO: 1419) | |
| 7E9 | QVQLQQSGPELVK | WVKQSHGKSLEW | QKFKGKATLTVDRS | WGQGTSVTVSS |
| | PGASVKISCKTSG | (SEQ ID NO: 996) | SSTAYMELRSLTSED | (SEQ ID NO: 1030) |
| | (SEQ ID NO: 1417) | | SAVYYC | |
| | | | (SEQ ID NO: 1420) | |
| SC3 | QVQLQQSGPDLVKP | WVKQSHGKSLEW | QKFKGKAILTVDKS | WGQGTTLTVSS |
| | GASVKISCKASG | (SEQ ID NO: 996) | SSTAYMELRSLTSED | (SEQ ID NO: 1031) |
| | (SEQ ID NO: 1418) | | SAVYYC | |
| | | | (SEQ ID NO: 1421) | |
| 1B4 | EVQLVESGGGLVKP | WVRQTPEKRLEW | DSMKGRFTISRDNA | WGQGTTLTVSS |
| | GGSLKLSCEA | (SEQ ID NO: 995) | KNFLYLQMSSLRSE | (SEQ ID NO: 1031) |
| | (SEQ ID NO: 1539) | | DTAMYYC | |
| | | | (SEQ ID NO: 1542) | |
| 6H2 | QVQLQESGPGLVQP | WIRQSPGKGLEW | AAFISRLNISKDNSK | WGQGTLVTVSA |
| | SQSLSIICTV | (SEQ ID NO: 1541) | SQVFFKMNSLQSDD | (SEQ ID NO: 1029) |
| | (SEQ ID NO: 1540) | | TAIYYC | |
| | | | (SEQ ID NO: 1543) | |
| 7B11v1 | EVQLVESGGGLVKP | WVRQTPEKRLEW | DSMKGRFTISRDNA | WGQGTTLTVSS |
| | GGSLKLSCEA | (SEQ ID NO: 995) | KNFLYLQMSSLRSE | (SEQ ID NO: 1031) |
| | (SEQ ID NO: 1539) | | DTAMYYC | |
| | | | (SEQ ID NO: 1542) | |
| 7B11v2 | EVQZQQSGPELVKP | WVKQSLGKSLEW | QKFKGKATLTVDKS | RGTGTTVTV |
| | GASVKISCKA | (SEQ ID NO: 1613) | SSTAYMELRSLTXEE | (SEQ ID NO: 1626) |
| | (SEQ ID NO: 1605) | | SAVYYC | |
| | | | (SEQ ID NO: 1618) | |
| 18D8 | EVQLVESGGGLVKP | WVRQTPEKRLEW | DSMKGRFTISRDNA | WGQGTTLTVSS |
| | GGSLKLSCEA | (SEQ ID NO: 995) | KNFLYLQMSSLRSE | (SEQ ID NO: 1031) |
| | (SEQ ID NO: 1539) | | DTAMYYC | |
| | | | (SEQ ID NO: 1542) | |
| 18E4v1 | EVQLVESGGGLVKP | WVRQTPEKRLEW | DSMKGRFTISRDNA | WGQGTTLTVSS |
| | GGSLKLSCEA | (SEQ ID NO: 995) | KNFLYLQMSSLRSE | (SEQ ID NO: 1031) |
| | (SEQ ID NO: 1539) | | DTAMYYC | |
| | | | (SEQ ID NO: 1542) | |
| 18E4v2 | QVQLQQPGAELVK | WVKEKPGQGLEW | HNFKGKATLTVDKS | WGQGTSVTVSS |
| | PGASVKVSCKA | (SEQ ID NO: 1614) | SSTAYMQLNSLTSE | (SEQ ID NO: 1030) |
| | (SEQ ID NO: 1606) | | DSAVYYC | |
| | | | (SEQ ID NO: 1619) | |
| 29F6v1 | EVQLVESGGGLVKP | WVRQTPEKRLEW | DSMKGRFTISRDNA | WGQGTTLTVSS |
| | GGSLKLSCEA | (SEQ ID NO: 995) | KNFLYLQMSSLRSE | (SEQ ID NO: 1031) |
| | (SEQ ID NO: 1539) | | DTAMYYC | |
| | | | (SEQ ID NO: 1542) | |
| 29F6v2 | QVQLQQSVAELVRP | WVKQRPEQGLEW | PKFQATATITVATSS | WGHGTSVTVSS |
| | GASVKLSCTA | (SEQ ID NO: 1615) | NSAYLQLSSLASEDT | (SEQ ID NO: 1627) |
| | (SEQ ID NO: 1607) | | AIYYC | |
| | | | (SEQ ID NO: 1620) | |
| 40D5v1 | EVQLVESGGGLVKP | WVRQTPEKRLEW | DSMKGRFTISRDNA | WGQGTTLTVSS |
| | GGSLKLSCEA | (SEQ ID NO: 995) | KNFLYLQMSSLRSE | (SEQ ID NO: 1031) |
| | (SEQ ID NO: 1539) | | DTAMYYC | |
| | | | (SEQ ID NO: 1542) | |
| 40D5v2 | QVQLQQSGAELVK | WVKQRPGQGLEW | QKFKGKATLTVDKS | WSQGTLVTVS |
| | PGASVKVSCKA | (SEQ ID NO: 1616) | SSTAYMQILSSLTSE | (SEQ ID NO: 1628) |
| | (SEQ ID NO: 1608) | | DSAVYYC | |
| | | | (SEQ ID NO: 1621) | |
| 43B9 | EVQLVESGGGLVKP | WVRQTPEKRLEW | DSMKGRFTISRDNA | WGQGTTLTVSS |
| | GGSLKLSCEA | (SEQ ID NO: 995) | KNFLYLQMSSLRSE | (SEQ ID NO: 1031) |
| | (SEQ ID NO: 1539) | | DTAMYYC | |
| | | | (SEQ ID NO: 1542) | |
| 44A8 | EVQLVESGGGLVKP | WVRQTPEKRLEW | DSMKGRFTISRDNA | WGQGTTLTVSS |
| | GGSLKLSCEA | (SEQ ID NO: 995) | KNFLYLQMSSLRSE | (SEQ ID NO: 1031) |
| | (SEQ ID NO: 1539) | | DTAMYYC | |
| | | | (SEQ ID NO: 1542) | |
| 44B4v1 | EVQLVESGGGLVKP | WVRQTPEKRLEW | DSMKGRFTISRDNA | WGQGTTLTVSS |
| | GGSLKLSCEA | (SEQ ID NO: 995) | KNFLYLQMSSLRSE | (SEQ ID NO: 1031) |
| | (SEQ ID NO: 1539) | | DTAMYYC | |
| | | | (SEQ ID NO: 1542) | |
| 44B4v2 | XXXXXQSGTELARP | WVKQRPGQGLEW | QKFKDKATLTADKS | WGTGTTVTVSS |
| | GASVKMPCKA | (SEQ ID NO: 1616) | SSTAYMQLSSLTSEE | (SEQ ID NO: 1033) |
| | (SEQ ID NO: 1609) | | SAVYYC | |
| | | | (SEQ ID NO: 1622) | |
| 45D6 | QVQLQQSGRELVKP | WVIQSHGESLEW | QKFKGKATLTVNKS | WGQGTSVTVSS |
| | GASVKMSCMSSG | (SEQ ID NO: 1617) | SSTAYMELRSLTSED | (SEQ ID NO: 1030) |
| | (SEQ ID NO: 1610) | | SAVYYC | |
| | | | (SEQ ID NO: 1623) | |
| 29F7 | QVKLEESGGGLVQP | WVRQSPEKGLEW | ESVKGRFTISRDDSK | WGQGTTLTVSS |
| | GGSMKLSCVASG | (SEQ ID NO: 998) | SSVYLQMNNLRAVD | (SEQ ID NO: 1031) |
| | (SEQ ID NO: 1611) | | TGIYYC | |
| | | | (SEQ ID NO: 1624) | |
| 32G1 | QVQLQQSGPELVKP | WVKQSHGKSLEW | QKFKGKATLTVNKS | WGQGTLVTVSA |
| | GASVQMSCEASG | (SEQ ID NO: 996) | SSTAYIELRSLTSEDS | (SEQ ID NO: 1029) |
| | (SEQ ID NO: 1612) | | AVYHC | |
| | | | (SEQ ID NO: 1625) | |

TABLE D7
Humanized light chain variable region sequences

| | |
|---|---|
| Antibody variant | Humanized sequences |

Antibody 4D11

| | |
|---|---|
| 4D11V3-15 | EIVMTQSPATLSVSPGERATLSCRASENIYSFLAWYQQKPGQAPRLLIY |
| | NSKTFAEGIPARFSGSGSGTEFTLTISSLQSEDFAVYYCQHHYGTPPWTF |
| | GQGTKVEIK (SEQ ID NO: 1040) |
| 4D11V1-9 | DIQLTQSPSFLSASVGDRVTITCRASENIYSFLAWYQQKPGKAPKLLI |
| | YNSKTFAEGVPSRFSGSGSGTEFTLTISSLQPEDFATYYCQHHYGTPP |
| | WTFGQGTKVEIK (SEQ ID NO: 1041) |
| 4D11V3-11 | EIVLTQSPATLSLSPGERATLSCRASENIYSFLAWYQQKPGQAPRLLIY |
| | NSKTFAEGIPARFSGSGSGTDFTLTISSLEPEDFAVYYCQHHYGTPPW |
| | TFGQGTKVEIK (SEQ ID NO: 1042) |
| 4D11V1-5 | DIQMTQSPSTLSASVGDRVTITCRASENIYSFLAWYQQKPGKAPKLLI |
| | YNSKTFAEGVPSRFSGSGSGTEFTLTISSLQPDDFATYYCQHHYGTPP |
| | WTFGQGTKVEIK (SEQ ID NO: 1043) |
| 4D11V1-39 | DIQMTQSPSSLSASVGDRVTITCRASENIYSFLAWYQQKPGKAPKLLI |
| | YNSKTFAEGVPSRFSGSGSGTDFTLTISSLQPEDFATYYCQHHYGTPP |
| | WTFGQGTKVEIK (SEQ ID NO: 1044) |
| 4D11V1-33 | DIQMTQSPSSLSASVGDRVTITCRASENIYSFLAWYQQKPGKAPKLLI |
| | YNSKTFAEGVPSRFSGSGSGTDFTFTISSLQPEDIATYYCQHHYGTPP |
| | WTFGQGTKVEIK (SEQ ID NO: 1045) |
| 4D11V3-20 | EIVLTQSPGTLSLSPGERATLSCRASENIYSFLAWYQQKPGQAPRLLIY |
| | NSKTFAEGIPDRFSGSGSGTDFTLTISRLEPEDFAVYYCQHHYGTPPW |
| | TFGQGTKVEIK (SEQ ID NO: 1046) |
| 4D11V2-28 | DIVMTQSPLSLPVTPGEPASISCRASENIYSFLAWYLQKPGQSPQLLIY |
| | NSKTFAEGVPDRFSGSGSGTDFTLKISRVEAEDVGVYYCQHHYGTPP |
| | WTFGQGTKVEIK (SEQ ID NO: 1047) |
| 4D11V2-30 | DVVMTQSPLSLPVTLGQPASISCRASENIYSFLAWFQQRPGQSPRRLI |
| | YNSKTFAEGVPDRFSGSGSGTDFTLKISRVEAEDVGVYYCQHHYGTP |
| | PWTFGQGTKVEIK (SEQ ID NO: 1048) |
| 4D11V4-1 | DIVMTQSPDSLAVSLGERATINCRASENIYSFLAWYQQKPGQPPKLLI |
| | YNSKTFAEGVPDRFSGSGSGTDFTLTISSLQAEDVAVYYCQHHYGTP |
| | PWTFGQGTKVEIK (SEQ ID NO: 1049) |

Antibody 7C5

| | |
|---|---|
| 7C5V3-15 | EIVMTQSPATLSVSPGERATLSCRASENIYSFLAWYQQKPGQAPRLLI |
| | YNSKTFAEGIPARFSGSGSGTEFTLTISSLQSEDFAVYYCQHHYGTPP |
| | WTFGQGTKVEIK (SEQ ID NO: 1040) |
| 7C5V1-9 | DIQLTQSPSFLSASVGDRVTITCRASENIYSFLAWYQQKPGKAPKLLI |
| | YNSKTFAEGVPSRFSGSGSGTEFTLTISSLQPEDFATYYCQHHYGTPP |
| | WTFGQGTKVEIK (SEQ ID NO: 1041) |
| 7C5V3-11 | EIVLTQSPATLSLSPGERATLSCRASENIYSFLAWYQQKPGQAPRLLIY |
| | NSKTFAEGIPARFSGSGSGTDFTLTISSLEPEDFAVYYCQHHYGTPPW |
| | TFGQGTKVEIK (SEQ ID NO: 1042) |
| 7C5V1-5 | DIQMTQSPSTLSASVGDRVTITCRASENIYSFLAWYQQKPGKAPKLLI |
| | YNSKTFAEGVPSRFSGSGSGTEFTLTISSLQPDDFATYYCQHHYGTPP |
| | WTFGQGTKVEIK (SEQ ID NO: 1043) |
| 7C5V1-39 | DIQMTQSPSSLSASVGDRVTITCRASENIYSFLAWYQQKPGKAPKLLI |
| | YNSKTFAEGVPSRFSGSGSGTDFTLTISSLQPEDFATYYCQHHYGTPP |
| | WTFGQGTKVEIK (SEQ ID NO: 1044) |
| 7C5V1-33 | DIQMTQSPSSLSASVGDRVTITCRASENIYSFLAWYQQKPGKAPKLLI |
| | YNSKTFAEGVPSRFSGSGSGTDFTFTISSLQPEDIATYYCQHHYGTPP |
| | WTFGQGTKVEIK (SEQ ID NO: 1045) |
| 7C5V3-20 | EIVLTQSPGTLSLSPGERATLSCRASENIYSFLAWYQQKPGQAPRLLIY |
| | NSKTFAEGIPDRFSGSGSGTDFTLTISRLEPEDFAVYYCQHHYGTPPW |
| | TFGQGTKVEIK (SEQ ID NO: 1046) |
| 7C5V2-28 | DIVMTQSPLSLPVTPGEPASISCRASENIYSFLAWYLQKPGQSPQLLIY |
| | NSKTFAEGVPDRFSGSGSGTDFTLKISRVEAEDVGVYYCQHHYGTPP |
| | WTFGQGTKVEIK (SEQ ID NO: 1047) |
| 7C5V2-30 | DVVMTQSPLSLPVTLGQPASISCRASENIYSFLAWFQQRPGQSPRRLI |
| | YNSKTFAEGVPDRFSGSGSGTDFTLKISRVEAEDVGVYYCQHHYGTP |
| | PWTFGQGTKVEIK (SEQ ID NO: 1048) |
| 7C5V4-1 | DIVMTQSPDSLAVSLGERATINCRASENIYSFLAWYQQKPGQPPKLLI |
| | YNSKTFAEGVPDRFSGSGSGTDFTLTISSLQAEDVAVYYCQHHYGTP |
| | PWTFGQGTKVEIK (SEQ ID NO: 1049) |

Antibody 6G12

| | |
|---|---|
| 6G12V4-1 | DIVMTQSPDSLAVSLGERATTNCKSSQSLLYSSNQKNCLAWYQQKPG |
| | QPPKLLIYWAFTRESGVPDRFSGSGSGTDFTLTISSLQAEDVAVYYCQ |
| | QYYSYPLTFGQGTKVEIK (SEQ ID NO: 1051) |
| 6G12V2-30 | DVVMTQSPLSLPVTLGQPASISCKSSQSLLYSSNQKNCLAWFQQRPG |
| | QSPRRLIYWAFTRESGVPDRFSGSGSGTDFTLKISRVEAEDVGVYYC |
| | QQYYSYPLTFGQGTKVEIK (SEQ ID NO: 1052) |
| 6G12V2-28 | DIVMTQSPLSLPVTPGEPASISCKSSQSLLYSSNQKNCLAWYLQKPG |
| | QSPQLLIYWAFTRESGVPDRFSGSGSGTDFTLKISRVEAEDVGVYYC |
| | QQYYSYPLTFGQGTKVEIK (SEQ ID NO: 1053) |
| 6G12V1-9 | DIQLTQSPSFLSASVGDRVTITCKSSQSLLYSSNQKNCLAWYQQKPG |
| | KAPKLLIYWAFTRESGVPSRFSGSGSGTEFTLTISSLQPEDFATYYCQ |
| | QYYSYPLTFGQGTKVEIK (SEQ ID NO: 1054) |
| 6G12V1-5 | DIQMTQSPSTLSASVGDRVTITCKSSQSLLYSSNQKNCLAWYQQKPG |
| | KAPKLLIYWAFTRESGVPSRFSGSGSGTEFTLTISSLQPDDFATYYCQ |
| | QYYSYPLTFGQGTKVEIK (SEQ ID NO: 1055) |
| 6G12V3-15 | EIVMTQSPATLSVSPGERATLSCKSSQSLLYSSNQKNCLAWYQQKPG |
| | QAPRLLIYWAFTRESGIPARFSGSGSGTEFTLTISSLQSEDFAVYYCQQ |
| | YYSYPLTFGQGTKVEIK (SEQ ID NO: 1056) |
| 6G12V1-33 | DIQMTQSPSSLSASVGDRVTITCKSSQSLLYSSNQKNCLAWYQQKPG |
| | KAPKLLIYWAFTRESGVPSRFSGSGSGTDFTFTISSLQPEDIATYYCQQ |
| | YYSYPLTFGQGTKVEIK (SEQ ID NO: 1057) |
| 6G12V1-39 | DIQMTQSPSSLSASVGDRVTITCKSSQSLLYSSNQKNCLAWYQQKPG |
| | KAPKLLIYWAFTRESGVPSRFSGSGSGTDFTLTISSLQPEDFATYYCQQ |
| | YYSYPLTFGQGTKVEIK (SEQ ID NO: 1058) |
| 6G12V3-11 | EIVLTQSPATLSLSPGERATLSCKSSQSLLYSSNQKNCLAWYQQKPG |
| | QAPRLLIYWAFTRESGIPARFSGSGSGTDFTLTISSLEPEDFAVYYCQQ |
| | YYSYPLTFGQGTKVEIK (SEQ ID NO: 1059) |
| 6G12V3-20 | EIVLTQSPGTLSLSPGERATLSCKSSQSLLYSSNQKNCLAWYQQKPG |
| | QAPRLLIYWAFTRESGIPDRFSGSGSGTDFTLTISRLEPEDFAVYYCQ |
| | QYYSYPLTFGQGTKVEIK (SEQ ID NO: 1060) |

Antibody 8F11

| | |
|---|---|
| 8F11V2-30 | DVVMTQSPLSLPVTLGQPASISCKSSQSLLYSNGKTFLSWFQQRPGQS |
| | PRRLIYLVSKLDSGVPDRFSGSGSGTDFTLKISRVEAEDVGVYYCMQ |
| | GTHFPLTFGQGTKVEIK (SEQ ID NO: 1062) |
| 8F11V2-28 | DIVMTQSPLSLPVTPGEPASISCKSSQSLLYSNGKTFLSWYLQKPGQS |
| | PQLLIYLVSKLDSGVPDRFSGSGSGTDFTLKISRVEAEDVGVYYCMQ |
| | GTHFPLTFGQGTKVEIK (SEQ ID NO: 1063) |
| 8F11V4-1 | DIVMTQSPDSLAVSLGERATINCKSSQSLLYSNGKTFLSWYQQKPGQ |
| | PPKWYLVSKLDSGVPDRFSGSGSGTDFTLTISSLQAEDVAVYYCMQ |
| | GTHFPLTFGQGTKVEIK (SEQ ID NO: 1064) |
| 8F11V1-5 | DIQMTQSPSTLSASVGDRVTITCKSSQSLLYSNGKTFLSWYQQKPGK |
| | APKWYLVSKLDSGVPSRFSGSGSGTEFTLTISSLQPDDFATYYCMQ |
| | GTHFPLTFGQGTKVEIK (SEQ ID NO: 1065) |
| 8F11V1-9 | DIQLTQSPSFLSASVGDRVTITCKSSQSLLYSNGKTFLSWYQQKPGK |
| | APKWYLVSKLDSGVPSRFSGSGSGTEFTLTISSLQPEDFATYYCMQ |
| | GTHFPLTFGQGTKVEIK (SEQ ID NO: 1066) |
| 8F11V1-39 | DIQMTQSPSSLSASVGDRVTITCKSSQSLLYSNGKTFLSWYQQKPGK |
| | APKWYLVSKLDSGVPSRFSGSGSGTDFTLTISSLQPEDFATYYCMQ |
| | GTHFPLTFGQGTKVEIK (SEQ ID NO: 1067) |
| 8F11V1-33 | DIQMTQSPSSLSASVGDRVTITCKSSQSLLYSNGKTFLSWYQQKPGK |
| | APKWYLVSKLDSGVPSRFSGSGSGTDFTFTISSLQPEDIATYYCMQG |
| | THFPLTFGQGTKVEIK (SEQ ID NO: 1068) |
| 8F11V3-15 | EIVMTQSPATLSVSPGERATLSCKSSQSLLYSNGKTFLSWYQQKPGQ |
| | APRLLIYLVSKLDSGIPARFSGSGSGTEFTLTISSLQSEDFAVYYCMQG |
| | THFPLTFGQGTKVEIK (SEQ ID NO: 1069) |
| 8F11V3-11 | EIVLTQSPATLSLSPGERATLSCKSSQSLLYSNGKTFLSWYQQKPGQ |
| | APRLLIYLVSKLDSGIPARFSGSGSGTDFTLTISSLEPEDFAVYYCMQ |
| | GTHFPLTFGQGTKVEIK (SEQ ID NO: 1070) |
| 8F11V3-20 | EIVLTQSPGTLSLSPGERATLSCKSSQSLLYSNGKTFLSWYQQKPGQA |
| | PRLLIYLVSKLDSGIPDRFSGSGSGTDFTLTISRLEPEDFAVYYCMQG |
| | THFPLTFGQGTKVEIK (SEQ ID NO: 1071) |

Antibody 8E10

| | |
|---|---|
| 8E10V2-30 | DVVMTQSPLSLPVTLGQPASISCKSSQSLLDSDGKTYLNWFQQRPGQ |
| | SPRRLIYLVSKLDSGVPDRFSGSGSGTDFTLKISRVEAEDVGVYYCWQ |
| | GTHFPYTFGQGTKVEIK (SEQ ID NO: 1073) |
| 8E10V2-28 | DIVMTQSPLSLPVTPGEPASISCKSSQSLLDSDGKTYLNWYLQKPGQS |
| | PQLLIYLVSKLDSGVPDRFSGSGSGTDFTLKISRVEAEDVGVYYCWQ |
| | GTHFPYTFGQGTKVEIK (SEQ ID NO: 1074) |
| 8E10V4-1 | DIVMTQSPDSLAVSLGERATINCKSSQSLLDSDGKTYLNWYQQKPGQ |
| | PPKWYLVSKLDSGVPDRFSGSGSGTDFTLTISSLQAEDVAVYYCWQ |
| | GTHFPYTFGQGTKVEIK (SEQ ID NO: 1075) |
| 8E10V1-9 | DIQLTQSPSFLSASVGDRVTITCKSSQSLLDSDGKTYLNWYQQKPGK |
| | APKWYLVSKLDSGVPSRFSGSGSGTEFTLTISSLQPEDFATYYCWQG |
| | THFPYTFGQGTKVEIK (SEQ ID NO: 1076) |
| 8E10V1-5 | DIQMTQSPSTLSASVGDRVTITCKSSQSLLDSDGKTYLNWYQQKPGK |
| | APKWYLVSKLDSGVPSRFSGSGSGTEFTLTISSLQPDDFATYYCWQ |
| | GTHFPYTFGQGTKVEIK (SEQ ID NO: 1077) |
| 8E10V1-39 | DIQMTQSPSSLSASVGDRVTITCKSSQSLLDSDGKTYLNWYQQKPGK |
| | APKWYLVSKLDSGVPSRFSGSGSGTDFTLTISSLQPEDFATYYCWQ |
| | GTHFPYTFGQGTKVEIK (SEQ ID NO: 1078) |
| 8E10V1-33 | DIQMTQSPSSLSASVGDRVTITCKSSQSLLDSDGKTYLNWYQQKPGK |
| | APKWYLVSKLDSGVPSRFSGSGSGTDFTFTISSLQPEDIATYYCWQ |
| | GTHFPYTFGQGTKVEIK (SEQ ID NO: 1079) |
| 8E10V3-11 | EIVLTQSPATLSLSPGERATLSCKSSQSLLDSDGKTYLNWYQQKPGQ |
| | APRLLIYLVSKLDSGIPARFSGSGSGTDFTLTISSLEPEDFAVYYCWQG |
| | THFPYTFGQGTKVEIK (SEQ ID NO: 1080) |
| 8E10V3-15 | EIVMTQSPATLSVSPGERATLSCKSSQSLLDSDGKTYLNWYQQKPGQ |
| | APRLLIYLVSKLDSGIPARFSGSGSGTEFTLTISSLQSEDFAVYYCWQ |
| | GTHFPYTFGQGTKVEIK (SEQ ID NO: 1081) |
| 8E10V3-20 | EIVLTQSPGTLSLSPGERATLSCKSSQSLLDSDGKTYLNWYQQKPGQ |
| | APRLLIYLVSKLDSGIPDRFSGSGSGTDFTLTISRLEPEDFAVYYCWQ |
| | GTHFPYTFGQGTKVEIK (SEQ ID NO: 1082) |

Antibody 7E5

| | |
|---|---|
| 7E5V2-30 | DVVMTQSPLSLPVTLGQPASISCKSSQSLLYSNGKTFLSWFQQRPGQS |
| | PRRLIYLVSKLDSGVPDRFSGSGSGTDFTLKISRVEAEDVGVYYCMQ |
| | GTHFPLTFGQGTKVEIK (SEQ ID NO: 1062) |
| 7E5V2-28 | DIVMTQSPLSLPVTPGEPASISCKSSQSLLYSNGKTFLSWYLQKPGQS |
| | PQLLIYLVSKLDSGVPDRFSGSGSGTDFTLKISRVEAEDVGVYYCMQ |
| | GTHFPLTFGQGTKVEIK (SEQ ID NO: 1063) |
| 7E5V4-1 | DIVMTQSPDSLAVSLGERATINCKSSQSLLYSNGKTFLSWYQQKPGQ |
| | PPKWYLVSKLDSGVPDRFSGSGSGTDFTLTISSLQAEDVAVYYCMQ |
| | GTHFPLTFGQGTKVEIK (SEQ ID NO: 1064) |
| 7E5V1-5 | DIQMTQSPSTLSASVGDRVTITCKSSQSLLYSNGKTFLSWYQQKPGK |
| | APKWYLVSKLDSGVPSRFSGSGSGTEFTLTISSLQPDDFATYYCMQG |
| | THFPLTFGQGTKVEIK (SEQ ID NO: 1065) |
| 7E5V1-9 | DIQLTQSPSFLSASVGDRVTITCKSSQSLLYSNGKTFLSWYQQKPGK |
| | APKLLIYLVSKLDSGVPSRFSGSGSGTEFTLTISSLQPEDFATYYCMQ |
| | GTHFPLTFGQGTKVEIK (SEQ ID NO: 1066) |
| 7E5V1-39 | DIQMTQSPSSLSASVGDRVTITCKSSQSLLYSNGKTFLSWYQQKPGKA |
| | PKWYLVSKLDSGVPSRFSGSGSGTDFTLTISSLQPEDFATYYCMQGT |
| | HFPLTFGQGTKVEIK (SEQ ID NO: 1067) |
| 7E5V1-33 | DIQMTQSPSSLSASVGDRVTITCKSSQSLLYSNGKTFLSWYQQKPGKA |
| | PKWYLVSKLDSGVPSRFSGSGSGTDFTFTISSLQPEDIATYYCMQGT |
| | HFPLTFGQGTKVEIK (SEQ ID NO: 1068) |
| 7E5V3-15 | EIVMTQSPATLSVSPGERATLSCKSSQSLLYSNGKTFLSWYQQKPGQ |
| | APRLLIYLVSKLDSGIPARFSGSGSGTEFTLTISSLQSEDFAVYYCMQG |
| | THFPLTFGQGTKVEIK (SEQ ID NO: 1069) |
| 7E5V3-11 | EIVLTQSPATLSLSPGERATLSCKSSQSLLYSNGKTFLSWYQQKPGQA |
| | PRLLIYLVSKLDSGIPARFSGSGSGTDFTLTISSLEPEDFAVYYCMQGT |
| | HFPLTFGQGTKVEIK (SEQ ID NO: 1070) |
| 7E5V3-20 | EIVLTQSPGTLSLSPGERATLSCKSSQSLLYSNGKTFLSWYQQKPGQA |
| | PRLLIYLVSKLDSGIPDRFSGSGSGTDFTLTISRLEPEDFAVYYCMQG |
| | THFPLTFGQGTKVEIK (SEQ ID NO: 1071) |

Antibody 7F8

| | |
|---|---|
| 7F8V3-11 | EIVLTQSPATLSLSPGERATLSCSASSSVSYMYWYQQKPGQAPRLLIYL |
| | TSILASGIPARFSGSGSGTDFTLTISSLEPEDFAVYYCQQWSFNPYTFGQ |
| | GTKVEIK (SEQ ID NO: 1084) |
| 7F8V1-39 | DIQMTQSPSSLSASVGDRVTITCSASSSVSYMYWYQQKPGKAPKLLI |
| | YLTSILASGVPSRFSGSGSGTDFTLTISSLQPEDFATYYCQQWSFNPYT |
| | FGQGTKVEIK (SEQ ID NO: 1085) |
| 7F8V1-5 | DIQMTQSPSTLSASVGDRVTITCSASSSVSYMYWYQQKPGKAPKLLI |
| | YLTSILASGVPSRFSGSGSGTEFTLTISSLQPDDFATYYCQQWSFNPYT |
| | FGQGTKVEIK (SEQ ID NO: 1086) |
| 7F8V3-15 | EIVMTQSPATLSVSPGERATLSCSASSSVSYMYWYQQKPGQAPRLLI |
| | YLTSILASGIPARFSGSGSGTEFTLTISSLQSEDFAVYYCQQWSFNPYT |
| | FGQGTKVEIK (SEQ ID NO: 1087) |
| 7F8V1-9 | DIQLTQSPSFLSASVGDRVTITCSASSSVSYMYWYQQKPGKAPKWYL |
| | TSILASGVPSRFSGSGSGTEFTLTISSLQPEDFATYYCQQWSFNPYTFGQ |
| | GTKVEIK (SEQ ID NO: 1088) |
| 7F8V1-33 | DIQMTQSPSSLSASVGDRVTITCSASSSVSYMYWYQQKPGKAPKLLI |
| | YLTSILASGVPSRFSGSGSGTDFTFTISSLQPEDIATYYCQQWSFNPYT |
| | FGQGTKVEIK (SEQ ID NO: 1089) |
| 7F8V3-20 | EIVLTQSPGTLSLSPGERATLSCSASSSVSYMYWYQQKPGQAPRLLIYL |
| | TSILASGIPDRFSGSGSGTDFTLTISRLEPEDFAVYYCQQWSFNPYTFGQ |
| | GTKVEIK (SEQ ID NO: 1090) |
| 7F8V2-28 | DIVMTQSPLSLPVTPGEPASISCSASSSVSYMYWYLQKPGQSPQLLIY |
| | LTSILASGVPDRFSGSGSGTDFTLKISRVEAEDVGVYYCQQWSFNPY |
| | TFGQGTKVEIK (SEQ ID NO: 1091) |
| 7F8V2-30 | DVVMTQSPLSLPVTLGQPASISCSASSSVSYMYWFQQRPGQSPRRLIY |
| | LTSILASGVPDRFSGSGSGTDFTLKISRVEAEDVGVYYCQQWSFNPYT |
| | FGQGTKVEIK (SEQ ID NO: 1092) |
| 7F8V4-1 | DIVMTQSPDSLAVSLGERATINCSASSSVSYMYWYQQKPGQPPKLLI |
| | YLTSILASGVPDRFSGSGSGTDFTLTISSLQAEDVAVYYCQQWSFNPY |
| | TFGQGTKVEIK (SEQ ID NO: 1093) |

Antibody 8F8

| | |
|---|---|
| 8F8V2-30 | DVVMTQSPLSLPVTLGQPASISCRSSQSLVHSNGNTYLHWFQQRPGQS |
| | PRRLIYKVSNRFSGVPDRFSGSGSGTDFTLKISRVEAEDVGVYYCSQS |
| | THVPLTFGQGTKVEIK (SEQ ID NO: 1095) |
| 8F8V2-28 | DIVMTQSPLSLPVTPGEPASISCRSSQSLVHSNGNTYLHWYLQKPGQ |
| | SPQLLIYKVSNRFSGVPDRFSGSGSGTDFTLKISRVEAEDVGVYYCSQ |
| | STHVPLTFGQGTKVEIK (SEQ ID NO: 1096) |
| 8F8V4-1 | DIVMTQSPDSLAVSLGERATINCRSSQSLVHSNGNTYLHWYQQKPGQ |
| | PPKWYKVSNRFSGVPDRFSGSGSGTDFTLTISSLQAEDVAVYYCSQS |
| | THVPLTFGQGTKVEIK (SEQ ID NO: 1097) |
| 8F8V3-11 | EIVLTQSPATLSLSPGERATLSCRSSQSLVHSNGNTYLHWYQQKPGQ |
| | APRLLIYKVSNRFSGIPARFSGSGSGTDFTLTISSLEPEDFAVYYCSQST |
| | HVPLTFGQGTKVEIK (SEQ ID NO: 1098) |
| 8F8V1-39 | DIQMTQSPSSLSASVGDRVTITCRSSQSLVHSNGNTYLHWYQQKPGK |
| | APKWYKVSNRFSGVPSRFSGSGSGTDFTLTISSLQPEDFATYYCSQS |
| | THVPLTFGQGTKVEIK (SEQ ID NO: 1099) |
| 8F8V1-33 | DIQMTQSPSSLSASVGDRVTITCRSSQSLVHSNGNTYLHWYQQKPGK |
| | APKLLIYKVSNRFSGVPSRFSGSGSGTDFTFTISSLQPEDIATYYCSQST |
| | HVPLTFGQGTKVEIK (SEQ ID NO: 1100) |
| 8F8V3-15 | EIVMTQSPATLSVSPGERATLSCRSSQSLVHSNGNTYLHWYQQKPGQA |
| | PRLLIYKVSNRFSGIPARFSGSGSGTEFTLTISSLQSEDFAVYYCSQSTHVP |
| | LTFGQGTKVEIK (SEQ ID NO: 1101) |
| 8F8V1-5 | DIQMTQSPSTLSASVGDRVTITCRSSQSLVHSNGNTYLHWYQQKPGK |
| | APKWYKVSNRFSGVPSRFSGSGSGTEFTLTISSLQPDDFATYYCSQS |
| | THVPLTFGQGTKVEIK (SEQ ID NO: 1102) |
| 8F8V1-9 | DIQLTQSPSFLSASVGDRVTITCRSSQSLVHSNGNTYLHWYQQKPGK |
| | APKWYKVSNRFSGVPSRFSGSGSGTEFTLTISSLQPEDFATYYCSQST |
| | HVPLTFGQGTKVEIK (SEQ ID NO: 1103) |
| 8F8V3-20 | EIVLTQSPGTLSLSPGERATLSCRSSQSLVHSNGNTYLHWYQQKPGQA |
| | PRLLIYKVSNRFSGIPDRFSGSGSGTDFTLTISRLEPEDFAVYYCSQSTH |
| | VPLTFGQGTKVEIK (SEQ ID NO: 1104) |

Antibody1H7

| | |
|---|---|
| 1H7V1-39 | DIQMTQSPSSLSASVGDRVTITCSASSSVSYMYWYQQKPGKAPKLLI |
| | YLTSILASGVPSRFSGSGSGTDFTLTISSLQPEDFATYYCQQWSFNPY |
| | TFGQGTKVEIK (SEQ ID NO: 1085) |
| 1H7V3-11 | EIVLTQSPATLSLSPGERATLSCSASSSVSYMYWYQQKPGQAPRLLIYL |
| | TSILASGIPARFSGSGSGTDFTLTISSLEPEDFAVYYCQQWSFNPYTFGQ |
| | GTKVEIK (SEQ ID NO: 1084) |
| 1H7V1-5 | DIQMTQSPSTLSASVGDRVTITCSASSSVSYMYWYQQKPGKAPKLLI |
| | YLTSILASGVPSRFSGSGSGTEFTLTISSLQPDDFATYYCQQWSFNP |
| | YTFGQGTK VEIK (SEQ ID NO: 1086) |
| 1H7V1-9 | DIQLTQSPSFLSASVGDRVTITCSASSSVSYMYWYQQKPGKAPKWYL |
| | TSILASGVPSRFSGSGSGTEFTLTISSLQPEDFATYYCQQWSFNPYTFGQ |
| | GTKVEIK (SEQ ID NO: 1088) |
| 1H7V3-15 | EIVMTQ SPATLSVSPGERATLSCSASSSVSYMYWYQQKPGQAPRLLI |
| | YLTSILASGIPARFSGSGSGTEFTLTISSLQSEDFAVYYCQQWSFNPYT |
| | FGQGTKVEIK (SEQ ID NO: 1087) |
| 1H7V1-33 | DIQMTQSPSSLSASVGDRVTITCSASSSVSYMYWYQQKPGKAPKLLI |
| | YLTSILASGVPSRFSGSGSGTDFTFTISSLQPEDIATYYCQQWSFNPYT |
| | FGQGTKVEIK (SEQ ID NO: 1089) |
| 1H7V3-20 | EIVLTQSPGTLSLSPGERATLSCSASSSVSYMYWYQQKPGQAPRLLIYL |
| | TSILASGIPDRFSGSGSGTDFTLTISRLEPEDFAVYYCQQWSFNPYTFGQ |
| | GTKVEIK (SEQ ID NO: 1090) |
| 1H7V2-28 | DIVMTQSPLSLPVTPGEPASISCSASSSVSYMYWYLQKPGQSPQLLIY |
| | LTSILASGVPDRFSGSGSGTDFTLKISRVEAEDVGVYYCQQWSFNPY |
| | TFGQGTKVEIK (SEQ ID NO: 1091) |
| 1H7V2-30 | DVVMTQSPLSLPVTLGQPASISCSASSSVSYMYWFQQRPGQSPRRLIY |
| | LTSILASGVPDRFSGSGSGTDFTLKISRVEAEDVGVYYCQQWSFNPYT |
| | FGQGTKVEIK (SEQ ID NO: 1092) |
| 1H7V4-1 | DIVMTQSPDSLAVSLGERATTNCSASSSVSYMYWYQQKPGQPPKLLI |
| | YLTSILASGVPDRFSGSGSGTDFTLTISSLQAEDVAVYYCQQWSFNPY |
| | TFGQGTKVEIK (SEQ ID NO: 1093) |

Antibody 2H8

| | |
|---|---|
| 2H8V3-11 | EIVLTQSPATLSLSPGERATLSCSASSSVSYMYWYQQKPGQAPRLLIYL |
| | TSILASGIPARFSGSGSGTDFTLTISSLEPEDFAVYYCQQWSFNPYTFGQ |
| | GTKVEIK (SEQ ID NO: 1084) |
| 2H8V1-39 | DIQMTQSPSSLSASVGDRVTITCSASSSVSYMYWYQQKPGKAPKLLI |
| | YLTSILASGVPSRFSGSGSGTDFTLTISSLQPEDFATYYCQQWSFNPY |
| | TFGQGTKVEIK (SEQ ID NO: 1085) |
| 2H8V1-5 | DIQMTQSPSTLSASVGDRVTITCSASSSVSYMYWYQQKPGKAPKLLI |
| | YLTSILASGVPSRFSGSGSGTEFTLTISSLQPDDFATYYCQQWSFNPYT |
| | FGQGTKVEIK (SEQ ID NO: 1086) |
| 2H8V3-15 | EIVMTQSPATLSVSPGERATLSCSASSSVSYMYWYQQKPGQAPRLLI |
| | YLTSILASGIPARFSGSGSGTEFTLTISSLQSEDFAVYYCQQWSFNPYT |
| | FGQGTKVEIK (SEQ ID NO: 1087) |
| 2H8V1-9 | DIQLTQSPSFLSASVGDRVTITCSASSSVSYMYWYQQKPGKAPKWYL |
| | TSILASGVPSRFSGSGSGTEFTLTISSLQPEDFATYYCQQWSFNPYTFGQ |
| | GTKVEIK (SEQ ID NO: 1088) |
| 2H8V1-33 | DIQMTQSPSSLSASVGDRVTITCSASSSVSYMYWYQQKPGKAPKLLI |
| | YLTSILASGVPSRFSGSGSGTDFTFTISSLQPEDIATYYCQQWSFNPYT |
| | FGQGTKVEIK (SEQ ID NO: 1089) |
| 2H8V3-20 | EIVLTQSPGTLSLSPGERATLSCSASSSVSYMYWYQQKPGQAPRLLIYL |
| | TSILASGIPDRFSGSGSGTDFTLTISRLEPEDFAVYYCQQWSFNPYTFGQ |
| | GTKVEIK (SEQ ID NO: 1090) |
| 2H8V2-28 | DIVMTQSPLSLPVTPGEPASISCSASSSVSYMYWYLQKPGQSPQLLIY |
| | LTSILASGVPDRFSGSGSGTDFTLKISRVEAEDVGVYYCQQWSFNPY |
| | TFGQGTKVEIK (SEQ ID NO: 1091) |
| 2H8V2-30 | DVVMTQSPLSLPVTLGQPASISCSASSSVSYMYWFQQRPGQSPRRLIY |
| | LTSILASGVPDRFSGSGSGTDFTLKISRVEAEDVGVYYCQQWSFNPYT |
| | FGQGTKVEIK (SEQ ID NO: 1092) |
| 2H8V4-1 | DIVMTQSPDSLAVSLGERATINCSASSSVSYMYWYQQKPGQPPKLLI |
| | YLTSILASGVPDRFSGSGSGTDFTLTISSLQAEDVAVYYCQQWSFNPY |
| | TFGQGTKVEIK (SEQ ID NO: 1093) |

Antibody 3A2

| | |
|---|---|
| 3A2 V2-30 | DVVMTQSPLSLPVTLGQPASISCRSSQTIIHSNGNTYLEWFQQRPGQS |
| | PRRLIYKVSNRFSGVPDRFSGSGSGTDFTLKISRVEAEDVGVYYCFQG |
| | SHVPYTFGQGTKVEIK (SEQ ID NO: 1108) |
| 3A2 V2-28 | DIVMTQSPLSLPVTPGEPASISCRSSQTIIHSNGNTYLEWYLQKPGQSP |
| | QLLIYKVSNRFSGVPDRFSGSGSGTDFTLKISRVEAEDVGVYYCFQGS |
| | HVPYTFGQGTKVEIK (SEQ ID NO: 1109) |
| 3A2 V4-1 | DIVMTQSPDSLAVSLGERATINCRSSQTIIHSNGNTYLEWYQQKPGQ |
| | PPKWYKVSNRFSGVPDRFSGSGSGTDFTLTISSLQAEDVAVYYCFQ |
| | GSHVPYTFGQGTKVEIK (SEQ ID NO: 1110) |
| 3A2 V3-11 | EIVLTQSPATLSLSPGERATLSCRSSQTIIHSNGNTYLEWYQQKPGQAP |
| | RLLIYKVSNRFSGIPARFSGSGSGTDFTLTISSLEPEDFAVYYCFQGSH |
| | VPYTFGQGTKVEIK (SEQ ID NO: 1111) |
| 3A2 I-9 | DIQLTQSPSFLSASVGDRVTITCRSSQTIIHSNGNTYLEWYQQKPGKA |
| | PKWYKVSNRFSGVPSRFSGSGSGTEFTLTISSLQPEDFATYYCFQGS |
| | HVPYTFGQGTKVEIK (SEQ ID NO: 1112) |
| 3A2 I-33 | DIQMTQSPSSLSASVGDRVTITCRSSQTIIHSNGNTYLEWYQQKPGK |
| | APKWYKVSNRFSGVPSRFSGSGSGTDFTFTISSLQPEDIATYYCFQG |
| | SHVPYTFGQGTKVEIK (SEQ ID NO: 1113) |
| 3A2 VI-39 | DIQMTQSPSSLSASVGDRVTITCRSSQTIIHSNGNTYLEWYQQKPGKA |
| | PKWYKVSNRFSGVPSRFSGSGSGTDFTLTISSLQPEDFATYYCFQGS |
| | HVPYTFGQGTKVEIK (SEQ ID NO: 1114) |
| 3A2 V3-15 | EIVMTQSPATLSVSPGERATLSCRSSQTIIHSNGNTYLEWYQQKPGQ |
| | APRLLIYKVSNRFSGIPARFSGSGSGTEFTLTISSLQSEDFAVYYCFQG |
| | SHVPYTFGQGTKVEIK (SEQ ID NO: 1115) |
| 3A2 VI-5 | DIQMTQSPSTLSASVGDRVTITCRSSQTIIHSNGNTYLEWYQQKPGK |
| | APKWYKVSNRFSGVPSRFSGSGSGTEFTLTISSLQPDDFATYYCFQG |
| | SHVPYTFGQGTKVEIK (SEQ ID NO: 1116) |
| 3A2 V3-20 | EIVLTQSPGTLSLSPGERATLSCRSSQTIIHSNGNTYLEWYQQKPGQA |
| | PRLLIYKVSNRFSGIPDRFSGSGSGTDFTLTISRLEPEDFAVYYCFQGS |
| | HVPYTFGQGTKVEIK (SEQ ID NO: 1117) |

Antibody 3A7

| | |
|---|---|
| 3A7 V2-30 | DVVMTQSPLSLPVTLGQPASISCKSSQSLLYSNGKTFLSWFQQRPGQS |
| | PRRLIYLVSKLDSGVPDRFSGSGSGTDFTLKISRVEAEDVGVYYCMQ |
| | GTHFPLTFGQGTKVEIK (SEQ ID NO: 1062) |
| 3A7 V2-28 | DIVMTQSPLSLPVTPGEPASISCKSSQSLLYSNGKTFLSWYLQKPGQS |
| | PQLLIYLVSKLDSGVPDRFSGSGSGTDFTLKISRVEAEDVGVYYCMQ |
| | GTHFPLTFGQGTKVEIK (SEQ ID NO: 1063) |
| 3A7 V4-1 | DIVMTQSPDSLAVSLGERATINCKSSQSLLYSNGKTFLSWYQQKPGQ |
| | PPKWYLVSKLDSGVPDRFSGSGSGTDFTLTISSLQAEDVAVYYCMQ |
| | GTHFPLTFGQGTKVEIK (SEQ ID NO: 1064) |
| 3A7 VI-39 | DIQMTQSPSSLSASVGDRVTITCKSSQSLLYSNGKTFLSWYQQKPGK |
| | APKLLIYLVSKLDSGVPSRFSGSGSGTDFTLTISSLQPEDFATYYCMQ |
| | GTHFPLTFGQGTKVEIK (SEQ ID NO: 1067) |
| 3A7 I-9 | DIQLTQSPSFLSASVGDRVTITCKSSQSLLYSNGKTFLSWYQQKPGK |
| | APKLLIYLVSKLDSGVPSRFSGSGSGTEFTLTISSLQPEDFATYYCMQ |
| | GTHFPLTFGQGTKVEIK (SEQ ID NO: 1066) |
| 3A7 I-5 | DIQMTQSPSTLSASVGDRVTITCKSSQSLLYSNGKTFLSWYQQKPGK |
| | APKLLIYLVSKLDSGVPSRFSGSGSGTEFTLTISSLQPDDFATYYCMQ |
| | GTHFPLTFGQGTKVEIK (SEQ ID NO: 1065) |
| 3A7 VI-33 | DIQMTQSPSSLSASVGDRVTITCKSSQSLLYSNGKTFLSWYQQKPGK |
| | APKLLIYLVSKLDSGVPSRFSGSGSGTDFTFTISSLQPEDIATYYCMQG |
| | THFPLTFGQGTKVEIK (SEQ ID NO: 1068) |
| 3A7 V3-15 | EIVMTQSPATLSVSPGERATLSCKSSQSLLYSNGKTFLSWYQQKPGQ |
| | APRLLIYLVSKLDSGIPARFSGSGSGTEFTLTISSLQSEDFAVYYCMQG |
| | THFPLTFGQGTKVEIK (SEQ ID NO: 1069) |
| 3A7 V3-11 | EIVLTQSPATLSLSPGERATLSCKSSQSLLYSNGKTFLSWYQQKPGQ |
| | APRLLIYLVSKLDSGIPARFSGSGSGTDFTLTISSLEPEDFAVYYCMQ |
| | GTHFPLTFGQGTKVEIK (SEQ ID NO: 1070) |
| 3A7 V3-20 | EIVLTQSPGTLSLSPGERATLSCKSSQSLLYSNGKTFLSWYQQKPGQ |
| | APRLLIYLVSKLDSGIPDRFSGSGSGTDFTLTISRLEPEDFAVYYCMQ |
| | GTHFPLTFGQGTKVEIK (SEQ ID NO: 1071) |

Antibody 3B10

| | |
|---|---|
| 3B10V4-1 | DIVMTQSPDSLAVSLGERATINCKSSQSLLYSSDQKNYLAWYQQKPG |
| | QPPKLLIYWASTRESGVPDRFSGSGSGTDFTLTISSLQAEDVAVYYCQ |
| | QYYSYPLTFGQGTKVEIK (SEQ ID NO: 1120) |
| 3B10V2-28 | DIVMTQSPLSLPVTPGEPASISCKSSQSLLYSSDQKNYLAWYLQKPG |
| | QSPQLLIYWASTRESGVPDRFSGSGSGTDFTLKISRVEAEDVGVYYC |
| | QQYYSYPLTFGQGTKVEIK (SEQ ID NO: 1121) |
| 3B10V2-30 | DVVMTQSPLSLPVTLGQPASISCKSSQSLLYSSDQKNYLAWFQQRPG |
| | QSPRRLIYWASTRESGVPDRFSGSGSGTDFTLKISRVEAEDVGVYYC |
| | QQYYSYPLTFGQGTKVEIK (SEQ ID NO: 1122) |
| 3B10V1-5 | DIQMTQSPSTLSASVGDRVTITCKSSQSLLYSSDQKNYLAWYQQKPG |
| | KAPKLLIYWASTRESGVPSRFSGSGSGTEFTLTISSLQPDDFATYYCQ |
| | QYYSYPLTFGQGTKVEIK (SEQ ID NO: 1123) |
| 3B10V1-9 | DIQLTQSPSFLSASVGDRVTITCKSSQSLLYSSDQKNYLAWYQQKPG |
| | KAPKLLIYWASTRESGVPSRFSGSGSGTEFTLTISSLQPEDFATYYCQQ |
| | YYSYPLTFGQGTKVEIK (SEQ ID NO: 1124) |
| 3B10V3-15 | EIVMTQSPATLSVSPGERATLSCKSSQSLLYSSDQKNYLAWYQQKPG |
| | QAPRLLIYWASTRESGIPARFSGSGSGTEFTLTISSLQSEDFAVYYCQQ |
| | YYSYPLTFGQGTKVEIK (SEQ ID NO: 1125) |
| 3B10V1-39 | DIQMTQSPSSLSASVGDRVTITCKSSQSLLYSSDQKNYLAWYQQKPG |
| | KAPKLLIYWASTRESGVPSRFSGSGSGTDFTLTISSLQPEDFATYYCQQ |
| | YYSYPLTFGQGTKVEIK (SEQ ID NO: 1126) |
| 3B10V3-11 | EIVLTQSPATLSLSPGERATLSCKSSQSLLYSSDQKNYLAWYQQKPG |
| | QAPRLLIYWASTRESGIPARFSGSGSGTDFTLTISSLEPEDFAVYYCQQ |
| | YYSYPLTFGQGTKVEIK (SEQ ID NO: 1127) |
| 3B10v1-33 | DIQMTQSPSSLSASVGDRVTITCKSSQSLLYSSDQKNYLAWYQQKPG |
| | KAPKLLIYWASTRESGVPSRFSGSGSGTDFTFTISSLQPEDIATYYCQQ |
| | YYSYPLTFGQGTKVEIK (SEQ ID NO: 1128) |
| 3B10V3-20 | EIVLTQSPGTLSLSPGERATLSCKSSQSLLYSSDQKNYLAWYQQKPG |
| | QAPRLLIYWASTRESGIPDRFSGSGSGTDFTLTISRLEPEDFAVYYCQ |
| | QYYSYPLTFGQGTKVEIK (SEQ ID NO: 1129) |

Antibody 4F11

| | |
|---|---|
| 4F11V2-30 | DVVMTQSPLSLPVTLGQPASISCRSSQTIIHSNGNTYLEWFQQRPGQS |
| | PRRLIYKVSNRFSGVPDRFSGSGSGTDFTLKISRVEAEDVGVYYCFQ |
| | GSHVPYTFGQGTKVEIK (SEQ ID NO: 1108) |
| 4F11V2-28 | DIVMTQSPLSLPVTPGEPASISCRSSQTIIHSNGNTYLEWYLQKPGQSP |
| | QLLIYKVSNRFSGVPDRFSGSGSGTDFTLKISRVEAEDVGVYYCFQG |
| | SHVPYTFGQGTKVEIK (SEQ ID NO: 1109) |
| 4F11V4-1 | DIVMTQSPDSLAVSLGERATINCRSSQTIIHSNGNTYLEWYQQKPGQPP |
| | KLLIYKVSNRFSGVPDRFSGSGSGTDFTLTISSLQAEDVAVYYCFQGSHV |
| | PYTFGQGTKVEIK(SEQ ID NO: 1110) |
| 4F11V3-11 | EIVLTQSPATLSLSPGERATLSCRSSQTIIHSNGNTYLEWYQQKPGQAP |
| | RLLIYKVSNRFSGIPARFSGSGSGTDFTLTISSLEPEDFAVYYCFQGSHV |
| | PYTFGQGTKVEIK (SEQ ID NO: 1111) |
| 4F11V3-15 | EIVMTQSPATLSVSPGERATLSCRSSQTIIHSNGNTYLEWYQQKPGQ |
| | APRLLIYKVSNRFSGIPARFSGSGSGTEFTLTISSLQSEDFAVYYCFQG |
| | SHVPYTFGQGTKVEIK (SEQ ID NO: 1115) |
| 4F11V1-33 | DIQMTQSPSSLSASVGDRVTITCRSSQTIIHSNGNTYLEWYQQKPGK |
| | APKWYKVSNRFSGVPSRFSGSGSGTDFTFTISSLQPEDIATYYCFQG |
| | SHVPYTFGQGTKVEIK (SEQ ID NO: 1113) |
| 4F11V1-39 | DIQMTQSPSSLSASVGDRVTITCRSSQTIIHSNGNTYLEWYQQKPGKA |
| | PKWYKVSNRFSGVPSRFSGSGSGTDFTLTISSLQPEDFATYYCFQGS |
| | HVPYTFGQGTKVEIK (SEQ ID NO: 1114) |
| 4F11V1-9 | DIQLTQSPSFLSASVGDRVTITCRSSQTIIHSNGNTYLEWYQQKPGKA |
| | PKWYKVSNRFSGVPSRFSGSGSGTEFTLTISSLQPEDFATYYCFQGS |
| | HVPYTFGQGTKVEIK (SEQ ID NO: 1112) |
| 4F11V1-5 | DIQMTQSPSTLSASVGDRVTITCRSSQTIIHSNGNTYLEWYQQKPGKA |
| | PKWYKVSNRFSGVPSRFSGSGSGTEFTLTISSLQPDDFATYYCFQGS |
| | HVPYTFGQGTKVEIK (SEQ ID NO: 1116) |
| 4F11V3-20 | EIVLTQSPGTLSLSPGERATLSCRSSQTIIHSNGNTYLEWYQQKPGQAP |
| | RLLIYKVSNRFSGIPDRFSGSGSGTDFTLTISRLEPEDFAVYYCFQGSH |
| | VPYTFGQGTKVEIK (SEQ ID NO: 1117) |

Antibody 6H6

| | |
|---|---|
| 6H6V4-1 | DIVMTQSPDSLAVSLGERATINCKSSQSVFYSSNQKNYLAWYQQKPG |
| | QPPKLLIYWASTRESGVPDRFSGSGSGTDFTLTISSLQAEDVAVYYCH |
| | QYLSSLTFGQGTKVEIK (SEQ ID NO: 1500) |
| 6H6V2-28 | DIVMTQSPLSLPVTPGEPASISCKSSQSVFYSSNQKNYLAWYLQKPGQ |
| | SPQLLIYWASTRESGVPDRFSGSGSGTDFTLKISRVEAEDVGVYYCHQ |
| | YLSSLTFGQGTKVEIK (SEQ ID NO: 1501) |
| 6H6V2-30 | DVVMTQSPLSLPVTLGQPASISCKSSQSVFYSSNQKNYLAWFQQRPG |
| | QSPRRLIYWASTRESGVPDRFSGSGSGTDFTLKISRVEAEDVGVYYCH |
| | QYLSSLTFGQGTKVEIK (SEQ ID NO: 1502) |
| 6H6V1-5 | DIQMTQSPSTLSASVGDRVTITCKSSQSVFYSSNQKNYLAWYQQKPG |
| | KAPKLLIYWASTRESGVPSRFSGSGSGTEFTLTISSLQPDDFATYYCH |
| | QYLSSLTFGQGTKVEIK (SEQ ID NO: 1503) |
| 6H6V1-9 | DIQLTQSPSFLSASVGDRVTITCKSSQSVFYSSNQKNYLAWYQQKPG |
| | KAPKLLIYWASTRESGVPSRFSGSGSGTEFTLTISSLQPEDFATYYCHQ |
| | YLSSLTFGQGTKVEIK (SEQ ID NO: 1504) |
| 6H6V3-15 | EIVMTQSPATLSVSPGERATLSCKSSQSVFYSSNQKNYLAWYQQKPG |
| | QAPRLLIYWASTRESGIPARFSGSGSGTEFTLTISSLQSEDFAVYYCHQ |
| | YLSSLTFGQGTKVEIK (SEQ ID NO: 1505) |
| 6H6V1-33 | DIQMTQSPSSLSASVGDRVTITCKSSQSVFYSSNQKNYLAWYQQKPG |
| | KAPKLLIYWASTRESGVPSRFSGSGSGTDFTFTISSLQPEDIATYYCHQ |
| | YLSSLTFGQGTKVEIK (SEQ ID NO: 1506) |
| 6H6V3-11 | EIVLTQSPATLSLSPGERATLSCKSSQSVFYSSNQKNYLAWYQQKPG |
| | QAPRLLIYWASTRESGIPARFSGSGSGTDFTLTISSLEPEDFAVYYCHQ |
| | YLSSLTFGQGTKVEIK (SEQ ID NO: 1507) |
| 6H6V1-39 | DIQMTQSPSSLSASVGDRVTITCKSSQSVFYSSNQKNYLAWYQQKPG |
| | KAPKLLIYWASTRESGVPSRFSGSGSGTDFTLTISSLQPEDFATYYCHQ |
| | YLSSLTFGQGTKVEIK (SEQ ID NO: 1508) |
| 6H6V3-20 | EIVLTQSPGTLSLSPGERATLSCKSSQSVFYSSNQKNYLAWYQQKPG |
| | QAPRLLIYWASTRESGIPDRFSGSGSGTDFTLTISRLEPEDFAVYYCHQ |
| | YLSSLTFGQGTKVEIK (SEQ ID NO: 1509) |

Antibody 7A9

| | |
|---|---|
| 7A9V1-9 | DIQLTQSPSFLSASVGDRVTITCRASENIYSYLAWYQQKPGKAPKLLI |
| | YKAKTLAEGVPSRFSGSGSGTEFTLTISSLQPEDFATYYCQHHYGTPF |
| | TFGQGTKVEIK (SEQ ID NO: 1132) |
| 7A9V3-11 | EIVLTQSPATLSLSPGERATLSCRASENIYSYLAWYQQKPGQAPRLLI |
| | YKAKTLAEGIPARFSGSGSGTDFTLTISSLEPEDFAVYYCQHHYGTPF |
| | TFGQGTKVEIK (SEQ ID NO: 1133) |
| 7A9V1-5 | DIQMTQSPSTLSASVGDRVTITCRASENIYSYLAWYQQKPGKAPKLL |
| | IYKAKTLAEGVPSRFSGSGSGTEFTLTISSLQPDDFATYYCQHHYGTP |
| | FTFGQGTKVEIK (SEQ ID NO: 1134) |
| 7A9V3-15 | EIVMTQSPATLSVSPGERATLSCRASENIYSYLAWYQQKPGQAPRLLI |
| | YKAKTLAEGIPARFSGSGSGTEFTLTISSLQSEDFAVYYCQHHYGTPF |
| | TFGQGTKVEIK (SEQ ID NO: 1135) |
| 7A9V1-39 | DIQMTQSPSSLSASVGDRVTITCRASENIYSYLAWYQQKPGKAPKLLI |
| | YKAKTLAEGVPSRFSGSGSGTDFTLTISSLQPEDFATYYCQHHYGTPF |
| | TFGQGTKVEIK (SEQ ID NO: 1136) |
| 7A9V1-33 | DIQMTQSPSSLSASVGDRVTITCRASENIYSYLAWYQQKPGKAPKLLI |
| | YKAKTLAEGVPSRFSGSGSGTDFTFTISSLQPEDIATYYCQHHYGTPFT |
| | FGQGTKVEIK (SEQ ID NO: 1137) |
| 7A9V3-20 | EIVLTQSPGTLSLSPGERATLSCRASENIYSYLAWYQQKPGQAPRLLI |
| | YKAKTLAEGIPDRFSGSGSGTDFTLTISRLEPEDFAVYYCQHHYGTPF |
| | TFGQGTKVEIK (SEQ ID NO: 1138) |
| 7A9V2-28 | DIVMTQSPLSLPVTPGEPASISCRASENIYSYLAWYLQKPGQSPQLLIY |
| | KAKTLAEGVPDRFSGSGSGTDFTLKISRVEAEDVGVYYCQHHYGTPF |
| | TFGQGTKVEIK (SEQ ID NO: 1139) |
| 7A9V4-1 | DIVMTQSPDSLAVSLGERATINCRASENIYSYLAWYQQKPGQPPKLL |
| | IYKAKTLAEGVPDRFSGSGSGTDFTLTISSLQAEDVAVYYCQHHYGT |
| | PFTFGQGTKVEIK (SEQ ID NO: 1140) |
| 7A9V2-30 | DVVMTQSPLSLPVTLGQPASISCRASENIYSYLAWFQQRPGQSPRRLI |
| | YKAKTLAEGVPDRFSGSGSGTDFTLKISRVEAEDVGVYYCQHHYGT |
| | PFTFGQGTKVEIK (SEQ ID NO: 1141) |

Antibody 8A1

| | |
|---|---|
| 8A1V3-15 | EIVMTQ SPATLSVSPGERATLSCRTSENVYSNLAWYQQKPGQAPRLL |
| | IYAATNLADGIPARFSGSGSGTEFTLTISSLQSEDFAVYYCHHFWGTP |
| | YTFGQGTKVEIK (SEQ ID NO: 1143) |
| 8A1V3-11 | EIVLTQ SPATLSLSPGERATLSCRTSENVYSNLAWYQQKPGQAPRLLI |
| | YAATNLADGIPARFSGSGSGTDFTLTISSLEPEDFAVYYCHHFWGTPY |
| | TFGQGTKVEIK (SEQ ID NO: 1144) |
| 8A1V1-9 | DIQLTQSPSFLSASVGDRVTITCRTSENVYSNLAWYQQKPGKAPKLLI |
| | YAATNLADGVPSRFSGSGSGTEFTLTISSLQPEDFATYYCHHFWGTPY |
| | TFGQGTKVEIK (SEQ ID NO: 1145) |
| 8A1V1-5 | DIQMTQSPSTLSASVGDRVTITCRTSENVYSNLAWYQQKPGKAPKLL |
| | IYAATNLADGVPSRFSGSGSGTEFTLTISSLQPDDFATYYCHHFWGTP |
| | YTFGQGTKVEIK (SEQ ID NO: 1146) |
| 8A1V1-39 | DIQMTQSPSSLSASVGDRVTITCRTSENVYSNLAWYQQKPGKAPKLL |
| | IYAATNLADGVPSRFSGSGSGTDFTLTISSLQPEDFATYYCHHFWGTP |
| | YTFGQGTKVEIK (SEQ ID NO: 1147) |
| 8A1V1-33 | DIQMTQSPSSLSASVGDRVTITCRTSENVYSNLAWYQQKPGKAPKLL |
| | IYAATNLADGVPSRFSGSGSGTDFTFTISSLQPEDIATYYCHHFWGTP |
| | YTFGQGTKVEIK (SEQ ID NO: 1148) |
| 8A1V3-20 | EIVLTQSPGTLSLSPGERATLSCRTSENVYSNLAWYQQKPGQAPRLLI |
| | YAATNLADGIPDRFSGSGSGTDFTLTISRLEPEDFAVYYCHHFWGTPY |
| | TFGQGTKVEIK (SEQ ID NO: 1149) |
| 8A1V2-28 | DIVMTQSPLSLPVTPGEPASISCRTSENVYSNLAWYLQKPGQSPQLLIY |
| | AATNLADGVPDRFSGSGSGTDFTLKISRVEAEDVGVYYCHHFWGTP |
| | YTFGQGTKVEIK (SEQ ID NO: 1150) |
| 8A1V2-30 | DVVMTQSPLSLPVTLGQPASISCRTSENVYSNLAWFQQRPGQSPRRLI |
| | YAATNLADGVPDRFSGSGSGTDFTLKISRVEAEDVGVYYCHHFWGTP |
| | YTFGQGTKVEIK (SEQ ID NO: 1151) |
| 8A1V4-1 | DIVMTQSPDSLAVSLGERATINCRTSENVYSNLAWYQQKPGQPPKLL |
| | IYAATNLADGVPDRFSGSGSGTDFTLTISSLQAEDVAVYYCHHFWGT |
| | PYTFGQGTKVEIK (SEQ ID NO: 1152) |

Antibody 9F5

| | |
|---|---|
| 9F5V2-30 | DVVMTQSPLSLPVTLGQPASISCRSSQSLVHSNGYTYLHWFQQRPGQ |
| | SPRRLIYKVSNRFSGVPDRFSGSGSGTDFTLKISRVEAEDVGVYYCSQ |
| | STRVPYTFGQGTKVEIK (SEQ ID NO: 1154) |
| 9F5V2-28 | DIVMTQSPLSLPVTPGEPASISCRSSQSLVHSNGYTYLHWYLQKPGQ |
| | SPQLLIYKVSNRFSGVPDRFSGSGSGTDFTLKISRVEAEDVGVYYCSQ |
| | STRVPYTFGQGTKVEIK (SEQ ID NO: 1155) |
| 9F5V4-1 | DIVMTQSPDSLAVSLGERATINCRSSQSLVHSNGYTYLHWYQQKPGQ |
| | PPKWYKVSNRFSGVPDRFSGSGSGTDFTLTISSLQAEDVAVYYCSQS |
| | TRVPYTFGQGTKVEIK (SEQ ID NO: 1156) |
| 9F5V3-11 | EIVLTQSPATLSLSPGERATLSCRSSQSLVHSNGYTYLHWYQQKPGQ |
| | APRLLIYKVSNRFSGIPARFSGSGSGTDFTLTISSLEPEDFAVYYCSQST |
| | RVPYTFGQGTKVEIK (SEQ ID NO: 1157) |
| 9F5V1-33 | DIQMTQSPSSLSASVGDRVTITCRSSQSLVHSNGYTYLHWYQQKPGK |
| | APKLLIYKVSNRFSGVPSRFSGSGSGTDFTFTISSLQPEDIATYYCSQST |
| | RVPYTFGQGTKVEIK (SEQ ID NO: 1158) |
| 9F5V3-15 | EIVMTQSPATLSVSPGERATLSCRSSQSLVHSNGYTYLHWYQQKPGQ |
| | APRLLIYKVSNRFSGIPARFSGSGSGTEFTLTISSLQSEDFAVYYCSQST |
| | RVPYTFGQGTKVEIK (SEQ ID NO: 1159) |
| 9F5V1-5 | DIQMTQSPSTLSASVGDRVTITCRSSQSLVHSNGYTYLHWYQQKPG |
| | KAPKWYKVSNRFSGVPSRFSGSGSGTEFTLTISSLQPDDFATYYCSQ |
| | STRVPYTFGQGTKVEIK (SEQ ID NO: 1160) |
| 9F5V1-39 | DIQMTQSPSSLSASVGDRVTITCRSSQSLVHSNGYTYLHWYQQKPGK |
| | APKWYKVSNRFSGVPSRFSGSGSGTDFTLTISSLQPEDFATYYCSQS |
| | TRVPYTFGQGTKVEIK (SEQ ID NO: 1161) |
| 9F5V1-9 | DIQLTQSPSFLSASVGDRVTITCRSSQSLVHSNGYTYLHWYQQKPGKA |
| | PKWYKVSNRFSGVPSRFSGSGSGTEFTLTISSLQPEDFATYYCSQSTR |
| | VPYTFGQGTKVEIK (SEQ ID NO: 1162) |
| 9F5V3-20 | EIVLTQSPGTLSLSPGERATLSCRSSQSLVHSNGYTYLHWYQQKPGQ |
| | APRLLIYKVSNRFSGIPDRFSGSGSGTDFTLTISRLEPEDFAVYYCSQST |
| | RVPYTFGQGTKVEIK (SEQ ID NO: 1163) |
| 9F5-L1 | DIVMTQTPLSLSVTPGQPASISCRSSQSLVHSNGYTYLHWYLQKPGQ |
| | SPQLLIYKVSNRFSGVPDRFSGSGSGTDFTLKISRVEAEDVGVYYCSQ |
| | STRVPYTFGQGTKLEIK (SEQ ID NO: 1663) |
| 9F5-L2 | DVVMTQTPLSLSVTPGQPASISCRSSQSLVHSNGYTYLHWYLQKPGQ |
| | SPQLLIYKVSNRFSGVPDRFSGSGSGTDFTLKISRVEAEDVGVYYCSQ |
| | STRVPYTFGQGTKLEIK (SEQ ID NO: 1664) |

Antibody 9G1

| | |
|---|---|
| 9G1V2-30 | DVVMTQSPLSLPVTLGQPASISCRFSQSLVHSNGNTYLHWFQQRPGQ |
| | SPRRLIYKVSNRFSGVPDRFSGSGSGTDFTLKISRVEAEDVGVYYCSQ |
| | STRVPPTFGQGTKVEIK (SEQ ID NO: 1165) |
| 9G1V2-28 | DIVMTQSPLSLPVTPGEPASISCRFSQSLVHSNGNTYLHWYLQKPGQSPQ |
| | LLIYKVSNRFSGVPDRFSGSGSGTDFTLKISRVEAEDVGVYYCSQSTRVP |
| | PTFGQGTKVEIK (SEQ ID NO: 1166) |
| 9G1V4-1 | DIVMTQSPDSLAVSLGERATINCRFSQSLVHSNGNTYLHWYQQKPGQ |
| | PPKWYKVSNRFSGVPDRFSGSGSGTDFTLTISSLQAEDVAVYYCSQS |
| | TRVPPTFGQGTKVEIK (SEQ ID NO: 1167) |
| 9G1V3 -11 | EIVLTQSPATLSLSPGERATLSCRFSQSLVHSNGNTYLHWYQQKPGQ |
| | APRLLIYKVSNRFSGIPARFSGSGSGTDFTLTISSLEPEDFAVYYCSQST |
| | RVPPTFGQGTKVEIK (SEQ ID NO: 1168) |
| 9G1V3-15 | EIVMTQSPATLSVSPGERATLSCRFSQSLVHSNGNTYLHWYQQKPGQ |
| | APRLLIYKVSNRFSGIPARFSGSGSGTEFTLTISSLQSEDFAVYYCSQS |
| | TRVPPTFGQGTKVEIK (SEQ ID NO: 1169) |
| 9G1V1-9 | DIQLTQSPSFLSASVGDRVTITCRFSQSLVHSNGNTYLHWYQQKPGK |
| | APKWYKVSNRFSGVPSRFSGSGSGTEFTLTISSLQPEDFATYYCSQST |
| | RVPPTFGQGTKVEIK (SEQ ID NO: 1170) |
| 9G1V1-5 | DIQMTQSPSTLSASVGDRVTITCRFSQSLVHSNGNTYLHWYQQKPGK |
| | APKWYKVSNRFSGVPSRFSGSGSGTEFTLTISSLQPDDFATYYCSQST |
| | RVPPTFGQGTKVEIK (SEQ ID NO: 1171) |
| 9G1V1-39 | DIQMTQSPSSLSASVGDRVTITCRFSQSLVHSNGNTYLHWYQQKPGKAP |
| | KLLIYKVSNRFSGVPSRFSGSGSGTDFTLTISSLQPEDFATYYCSQSTRVPP |
| | TFGQGTKVEIK (SEQ ID NO: 1172) |
| 9G1V1-33 | DIQMTQSPSSLSASVGDRVTITCRFSQSLVHSNGNTYLHWYQQKPGK |
| | APKWYKVSNRFSGVPSRFSGSGSGTDFTFTISSLQPEDIATYYCSQST |
| | RVPPTFGQGTKVEIK (SEQ ID NO: 1173) |
| 9G1V3 -20 | EIVLTQSPGTLSLSPGERATLSCRFSQSLVHSNGNTYLHWYQQKPGQ |
| | APRLLIYKVSNRFSGIPDRFSGSGSGTDFTLTISRLEPEDFAVYYCSQS |
| | TRVPPTFGQGTKVEIK (SEQ ID NO: 1174) |

Antibody 9G3

| | |
|---|---|
| 9G3V1-33 | DIQMTQSPSSLSASVGDRVTITCKASSNVNYMSWYQQKPGKAPKLLIY |
| | FTSNLPSGVPSRFSGSGSGTDFTFTISSLQPEDIATYYCSGEVTQFTFGQG |
| | TKVEIK (SEQ ID NO: 1176) |
| 9G3V1-9 | DIQLTQSPSFLSASVGDRVTITCKASSNVNYMSWYQQKPGKAPKLLIY |
| | FTSNLPSGVPSRFSGSGSGTEFTLTISSLQPEDFATYYCSGEVTQFTFGQ |
| | GTKVEIK (SEQ ID NO: 1177) |
| 9G3V1-39 | DIQMTQSPSSLSASVGDRVTITCKASSNVNYMSWYQQKPGKAPKLLIY |
| | FTSNLPSGVPSRFSGSGSGTDFTLTISSLQPEDFATYYCSGEVTQFTFGQ |
| | GTKVEIK (SEQ ID NO: 1178) |
| 9G3V3-11 | EIVLTQSPATLSLSPGERATLSCKASSNVNYMSWYQQKPGQAPRLLIYF |
| | TSNLPSGIPARFSGSGSGTDFTLTISSLEPEDFAVYYCSGEVTQFTFGQG |
| | TKVEIK (SEQ ID NO: 1641) |
| 9G3V1-5 | DIQMTQSPSTLSASVGDRVTITCKASSNVNYMSWYQQKPGKAPKLLIYF |
| | TSNLPSGVPSRFSGSGSGTEFTLTISSLQPDDFATYYCSGEVTQFTFGQGT |
| | KVEIK (SEQ ID NO: 1179) |
| 9G3V3-15 | EIVMTQSPATLSVSPGERATLSCKASSNVNYMSWYQQKPGQAPRLLIY |
| | FTSNLPSGIPARFSGSGSGTEFTLTISSLQSEDFAVYYCSGEVTQFTFGQG |
| | TKVEIK (SEQ ID NO: 1180) |
| 9G3V3-20 | EIVLTQSPGTLSLSPGERATLSCKASSNVNYMSWYQQKPGQAPRLLIYF |
| | TSNLPSGIPDRFSGSGSGTDFTLTISRLEPEDFAVYYCSGEVTQFTFGQG |
| | TKVEIK (SEQ ID NO: 1181) |
| 9G3V2-28 | DIVMTQSPLSLPVTPGEPASISCKASSNVNYMSWYLQKPGQSPQLLIY |
| | FTSNLPSGVPDRFSGSGSGTDFTLKISRVEAEDVGVYYCSGEVTQFTF |
| | GQGTKVEIK (SEQ ID NO: 1182) |
| 9G3V2-30 | DVVMTQSPLSLPVTLGQPASISCKASSNVNYMSWFQQRPGQSPRRLIYF |
| | TSNLPSGVPDRFSGSGSGTDFTLKISRVEAEDVGVYYCSGEVTQFTFGQ |
| | GTKVEIK (SEQ ID NO: 1183) |
| 9G3V4-1 | DIVMTQSPDSLAVSLGERATTNCKASSNVNYMSWYQQKPGQPPKLLI |
| | YFTSNLPSGVPDRFSGSGSGTDFTLTISSLQAEDVAVYYCSGEVTQFTF |
| | GQGTKVEIK (SEQ ID NO: 1184) |

Antibody10A9

| | |
|---|---|
| 10A9V2-30 | DVVMTQSPLSLPVTLGQPASISCRSSQTIIHSNGNTYLEWFQQRPGQSPRR |
| | LIYKVSNRFCGVPDRFSGSGSGTDFTLKISRVEAEDVGVYYCFQGSHVPY |
| | TFGQGTKVEIK (SEQ ID NO: 1186) |
| 10A9V2-28 | DIVMTQSPLSLPVTPGEPASISCRSSQTIIHSNGNTYLEWYLQKPGQSP |
| | QLLIYKVSNRFCGVPDRFSGSGSGTDFTLKISRVEAEDVGVYYCFQG |
| | SHVPYTFGQGTKVEIK (SEQ ID NO: 1187) |
| 10A9V4-1 | DIVMTQSPDSLAVSLGERATINCRSSQTIIHSNGNTYLEWYQQKPGQP |
| | PKWYKVSNRFCGVPDRFSGSGSGTDFTLTISSLQAEDVAVYYCFQG |
| | SHVPYTFGQGTKVEIK (SEQ ID NO: 1188) |
| 10A9V3-11 | EIVLTQSPATLSLSPGERATLSCRSSQTIIHSNGNTYLEWYQQKPGQAP |
| | RLLIYKVSNRFCGIPARFSGSGSGTDFTLTISSLEPEDFAVYYCFQGSH |
| | VPYTFGQGTKVEIK (SEQ ID NO: 1189) |
| 10A9V3-15 | EIVMTQSPATLSVSPGERATLSCRSSQTIIHSNGNTYLEWYQQKPGQ |
| | APRLLIYKVSNRFCGIPARFSGSGSGTEFTLTISSLQSEDFAVYYCFQG |
| | SHVPYTFGQGTKVEIK (SEQ ID NO: 1190) |
| 10A9V1-33 | DIQMTQSPSSLSASVGDRVTITCRSSQTIIHSNGNTYLEWYQQKPG |
| | KAPKWYKVSNRFCGVPSRFSGSGSGTDFTFTISSLQPEDIATYYCF |
| | QGSHVPYTFGQGTKVEIK (SEQ ID NO: 1191) |
| 10A9V3-20 | EIVLTQSPGTLSLSPGERATLSCRSSQTIIHSNGNTYLEWYQQKPGQAP |
| | RLLIYKVSNRFCGIPDRFSGSGSGTDFTLTISRLEPEDFAVYYCFQGSH |
| | VPYTFGQGTKVEIK (SEQ ID NO: 1192) |
| 10A9V1-9 | DIQLTQSPSFLSASVGDRVTITCRSSQTIIHSNGNTYLEWYQQKPGKA |
| | PKWYKVSNRFCGVPSRFSGSGSGTEFTLTISSLQPEDFATYYCFQGS |
| | HVPYTFGQGTKVEIK (SEQ ID NO: 1193) |
| 10A9V1-5 | DIQMTQSPSTLSASVGDRVTITCRSSQTIIHSNGNTYLEWYQQKPGKA |
| | PKWYKVSNRFCGVPSRFSGSGSGTEFTLTISSLQPDDFATYYCFQGS |
| | HVPYTFGQGTKVEIK (SEQ ID NO: 1194) |
| 10A9V1-39 | DIQMTQSPSSLSASVGDRVTITCRSSQTIIHSNGNTYLEWYQQKPGKA |
| | PKWYKVSNRFCGVPSRFSGSGSGTDFTLTISSLQPEDFATYYCFQGS |
| | HVPYTFGQGTKVEIK (SEQ ID NO: 1195) |

Antibody11A8

| | |
|---|---|
| 11A8V4-1 | DIVMTQSPDSLAVSLGERATINCKSSQSLLNSGNQKKYLTWYQQKPG |
| | QPPKLLIYWASTRESGVPDRFSGSGSGTDFTLTISSLQAEDVAVYYCQ |
| | NDYGFPLTFGQGTKVEIK (SEQ ID NO: 1197) |
| 11A8V2-30 | DVVMTQSPLSLPVTLGQPASISCKSSQSLLNSGNQKKYLTWFQQRPG |
| | QSPRRLIYWASTRESGVPDRFSGSGSGTDFTLKISRVEAEDVGVYYCQ |
| | NDYGFPLTFGQGTKVEIK (SEQ ID NO: 1198) |
| 11A8V2-28 | DIVMTQSPLSLPVTPGEPASISCKSSQSLLNSGNQKKYLTWYLQKPG |
| | QSPQLLIYWASTRESGVPDRFSGSGSGTDFTLKISRVEAEDVGVYYC |
| | QNDYGFPLTFGQGTKVEIK (SEQ ID NO: 1199) |
| 11A8V1-33 | DIQMTQSPSSLSASVGDRVTITCKSSQSLLNSGNQKKYLTWYQQKPG |
| | KAPKLLIYWASTRESGVPSRFSGSGSGTDFTFTISSLQPEDIATYYCQN |
| | DYGFPLTFGQGTKVEIK (SEQ ID NO: 1200) |
| 11A8V3-11 | EIVLTQSPATLSLSPGERATLSCKSSQSLLNSGNQKKYLTWYQQKPG |
| | QAPRLLIYWASTRESGIPARFSGSGSGTDFTLTISSLEPEDFAVYYCQN |
| | DYGFPLTFGQGTKVEIK (SEQ ID NO: 1201) |
| 11A8V3-15 | EIVMTQSPATLSVSPGERATLSCKSSQSLLNSGNQKKYLTWYQQKPG |
| | QAPRLLIYWASTRESGIPARFSGSGSGTEFTLTISSLQSEDFAVYYCQN |
| | DYGFPLTFGQGTKVEIK (SEQ ID NO: 1202) |
| 11A8V1-5 | DIQMTQSPSTLSASVGDRVTITCKSSQSLLNSGNQKKYLTWYQQKPG |
| | KAPKLLIYWASTRESGVPSRFSGSGSGTEFTLTISSLQPDDFATYYCQ |
| | NDYGFPLTFGQGTKVEIK (SEQ ID NO: 1203) |
| 11A8V3-20 | EIVLTQSPGTLSLSPGERATLSCKSSQSLLNSGNQKKYLTWYQQKPG |
| | QAPRLLIYWASTRESGIPDRFSGSGSGTDFTLTISRLEPEDFAVYYCQN |
| | DYGFPLTFGQGTKVEIK (SEQ ID NO: 1204) |
| 11A8V1-9 | DIQLTQSPSFLSASVGDRVTITCKSSQSLLNSGNQKKYLTWYQQKPG |
| | KAPKLLIYWASTRESGVPSRFSGSGSGTEFTLTISSLQPEDFATYYCQ |
| | NDYGFPLTFGQGTKVEIK (SEQ ID NO: 1205) |
| 11A8V1-39 | DIQMTQSPSSLSASVGDRVTITCKSSQSLLNSGNQKKYLTWYQQKPG |
| | KAPKLLIYWASTRESGVPSRFSGSGSGTDFTLTISSLQPEDFATYYCQ |
| | NDYGFPLTFGQGTKVEIK (SEQ ID NO: 1206) |

Antibody12D9

| | |
|---|---|
| 12D9V4-1 | DIVMTQSPDSLAVSLGERATINCKSSQSLLYSGNQKNFLAWYQQKPG |
| | QPPKLLIYWASTRESGVPDRFSGSGSGTDFTLTISSLQAEDVAVYYCQ |
| | QYYSYPFTFGQGTKVEIK (SEQ ID NO: 1208) |
| 12D9V2-28 | DIVMTQSPLSLPVTPGEPASISCKSSQSLLYSGNQKNFLAWYLQKPGQ |
| | SPQLLIYWASTRESGVPDRFSGSGSGTDFTLKISRVEAEDVGVYYCQQ |
| | YYSYPFTFGQGTKVEIK (SEQ ID NO: 1209) |
| 12D9V2-30 | DVVMTQSPLSLPVTLGQPASISCKSSQSLLYSGNQKNFLAWFQQRPG |
| | QSPRRLIYWASTRESGVPDRFSGSGSGTDFTLKISRVEAEDVGVYYCQ |
| | QYYSYPFTFGQGTKVEIK (SEQ ID NO: 1210) |
| 12D 9V1-9 | DIQLTQSPSFLSASVGDRVTITCKSSQSLLYSGNQKNFLAWYQQKPG |
| | KAPKLLIYWASTRESGVPSRFSGSGSGTEFTLTISSLQPEDFATYYCQQ |
| | YYSYPFTFGQGTKVEIK (SEQ ID NO: 1211) |
| 12D9V1-5 | DIQMTQSPSTLSASVGDRVTITCKSSQSLLYSGNQKNFLAWYQQKPG |
| | KAPKLLIYWASTRESGVPSRFSGSGSGTEFTLTISSLQPDDFATYYCQ |
| | QYYSYPFTFGQGTKVEIK (SEQ ID NO: 1212) |
| 12D9V3-15 | EIVMTQSPATLSVSPGERATLSCKSSQSLLYSGNQKNFLAWYQQKPG |
| | QAPRLLIYWASTRESGIPARFSGSGSGTEFTLTISSLQSEDFAVYYCQQ |
| | YYSYPFTFGQGTKVEIK (SEQ ID NO: 1213) |
| 12D9V1-33 | DIQMTQSPSSLSASVGDRVTITCKSSQSLLYSGNQKNFLAWYQQKPG |
| | KAPKLLIYWASTRESGVPSRFSGSGSGTDFTFTISSLQPEDIATYYCQQ |
| | YYSYPFTFGQGTKVEIK (SEQ ID NO: 1214) |
| 12D9V3-11 | EIVLTQSPATLSLSPGERATLSCKSSQSLLYSGNQKNFLAWYQQKPG |
| | QAPRLLIYWASTRESGIPARFSGSGSGTDFTLTISSLEPEDFAVYYCQQ |
| | YYSYPFTFGQGTKVEIK (SEQ ID NO: 1215) |
| 12D9V1-39 | DIQMTQSPSSLSASVGDRVTITCKSSQSLLYSGNQKNFLAWYQQKPG |
| | KAPKLLIYWASTRESGVPSRFSGSGSGTDFTLTISSLQPEDFATYYCQ |
| | QYYSYPFTFGQGTKVEIK (SEQ ID NO: 1216) |
| 12D9V3-20 | EIVLTQSPGTLSLSPGERATLSCKSSQSLLYSGNQKNFLAWYQQKPG |
| | QAPRLLIYWASTRESGIPDRFSGSGSGTDFTLTISRLEPEDFAVYYCQQ |
| | YYSYPFTFGQGTKVEIK (SEQ ID NO: 1217) |

Antibody12F9

| | |
|---|---|
| 12F9V4-1 | DIVMTQSPDSLAVSLGERATINCKSSQSLLYSSDQKNYLAWYQQKPG |
| | QPPKLLIYWASTRESGVPDRFSGSGSGTDFTLTISSLQAEDVAVYYCQ |
| | QYYSYPLTFGQGTKVEIK (SEQ ID NO: 1120) |
| 12F9V2-28 | DIVMTQSPLSLPVTPGEPASISCKSSQSLLYSSDQKNYLAWYLQKPG |
| | QSPQLLIYWASTRESGVPDRFSGSGSGTDFTLKISRVEAEDVGVYYC |
| | QQYYSYPLTFGQGTKVEIK (SEQ ID NO: 1121) |
| 12F9V2-30 | DVVMTQSPLSLPVTLGQPASISCKSSQSLLYSSDQKNYLAWFQQRPG |
| | QSPRRLIYWASTRESGVPDRFSGSGSGTDFTLKISRVEAEDVGVYYC |
| | QQYYSYPLTFGQGTKVEIK (SEQ ID NO: 1122) |
| 12F9V3-15 | EIVMTQSPATLSVSPGERATLSCKSSQSLLYSSDQKNYLAWYQQKPG |
| | QAPRLLIYWASTRESGIPARFSGSGSGTEFTLTISSLQSEDFAVYYCQQ |
| | YYSYPLTFGQGTKVEIK (SEQ ID NO: 1125) |
| 12F9V1-5 | DIQMTQSPSTLSASVGDRVTITCKSSQSLLYSSDQKNYLAWYQQKPG |
| | KAPKLLIYWASTRESGVPSRFSGSGSGTEFTLTISSLQPDDFATYYCQ |
| | QYYSYPLTFGQGTKVEIK (SEQ ID NO: 1123) |
| 12F9V1-9 | DIQLTQSPSFLSASVGDRVTITCKSSQSLLYSSDQKNYLAWYQQKPG |
| | KAPKLLIYWASTRESGVPSRFSGSGSGTEFTLTISSLQPEDFATYYCQ |
| | QYYSYPLTFGQGTKVEIK (SEQ ID NO: 1124) |
| 12F9V1-33 | DIQMTQSPSSLSASVGDRVTITCKSSQSLLYSSDQKNYLAWYQQKPG |
| | KAPKLLIYWASTRESGVPSRFSGSGSGTDFTFTISSLQPEDIATYYCQQ |
| | YYSYPLTFGQGTKVEIK (SEQ ID NO: 1128) |
| 12F9V3-11 | EIVLTQSPATLSLSPGERATLSCKSSQSLLYSSDQKNYLAWYQQKPG |
| | QAPRLLIYWASTRESGIPARFSGSGSGTDFTLTISSLEPEDFAVYYCQQ |
| | YYSYPLTFGQGTKVEIK (SEQ ID NO: 1127) |
| 12F9V1-39 | DIQMTQSPSSLSASVGDRVTITCKSSQSLLYSSDQKNYLAWYQQKPG |
| | KAPKLLIYWASTRESGVPSRFSGSGSGTDFTLTISSLQPEDFATYYCQQ |
| | YYSYPLTFGQGTKVEIK (SEQ ID NO: 1126) |
| 12F9V3-20 | EIVLTQSPGTLSLSPGERATLSCKSSQSLLYSSDQKNYLAWYQQKPG |
| | QAPRLLIYWASTRESGIPDRFSGSGSGTDFTLTISRLEPEDFAVYYCQQ |
| | YYSYPLTFGQGTKVEIK (SEQ ID NO: 1129) |

Antibodyl0C1

| | |
|---|---|
| 10C1V4-1 | DIVMTQSPDSLAVSLGERATINCKSSQSVFYSSNQKNYLAWYQQKPG |
| | QPPKLLIYWASTRESGVPDRFSGSGSGTDFTLTISSLQAEDVAVYYCH |
| | QYLSSLTFGQGTKVEIK (SEQ ID NO: 1423) |
| 10C1V2-30 | DVVMTQSPLSLPVTLGQPASISCKSSQSVFYSSNQKNYLAWFQQRPG |
| | QSPRRLIYWASTRESGVPDRFSGSGSGTDFTLKISRVEAEDVGVYYCH |
| | QYLSSLTFGQGTKVEIK (SEQ ID NO: 1424) |
| 10C1V2-28 | DIVMTQSPLSLPVTPGEPASISCKSSQSVFYSSNQKNYLAWYLQKPGQ |
| | SPQLLIYWASTRESGVPDRFSGSGSGTDFTLKISRVEAEDVGVYYCH |
| | QYLSSLTFGQGTKVEIK (SEQ ID NO: 1425) |
| 10C1V1-5 | DIQMTQSPSTLSASVGDRVTITCKSSQSVFYSSNQKNYLAWYQQKPG |
| | KAPKLLIYWASTRESGVPSRFSGSGSGTEFTLTISSLQPDDFATYYCHQ |
| | YLSSLTFGQGTKVEIK (SEQ ID NO: 1426) |
| 10C1V3-15 | EIVMTQSPATLSVSPGERATLSCKSSQSVFYSSNQKNYLAWYQQKPG |
| | QAPRLLIYWASTRESGIPARFSGSGSGTEFTLTISSLQSEDFAVYYCHQ |
| | YLSSLTFGQGTKVEIK (SEQ ID NO: 1427) |
| 10C1V1-9 | DIQLTQSPSFLSASVGDRVTITCKSSQSVFYSSNQKNYLAWYQQKPG |
| | KAPKLLIYWASTRESGVPSRFSGSGSGTEFTLTISSLQPEDFATYYCH |
| | QYLSSLTFGQGTKVEIK (SEQ ID NO: 1428) |
| 10C1V3-11 | EIVLTQSPATLSLSPGERATLSCKSSQSVFYSSNQKNYLAWYQQKPG |
| | QAPRLLIYWASTRESGIPARFSGSGSGTDFTLTISSLEPEDFAVYYCHQ |
| | YLSSLTFGQGTKVEIK (SEQ ID NO: 1429) |
| 10C1V1-39 | DIQMTQSPSSLSASVGDRVTITCKSSQSVFYSSNQKNYLAWYQQKPG |
| | KAPKLLIYWASTRESGVPSRFSGSGSGTDFTLTISSLQPEDFATYYCHQ |
| | YLSSLTFGQGTKVEIK (SEQ ID NO: 1430) |
| 10C1V1-33 | DIQMTQSPSSLSASVGDRVTITCKSSQSVFYSSNQKNYLAWYQQKPG |
| | KAPKLLIYWASTRESGVPSRFSGSGSGTDFTFTISSLQPEDIATYYCHQ |
| | YLSSLTFGQGTKVEIK (SEQ ID NO: 1431) |
| 10C1V3-20 | EIVLTQSPGTLSLSPGERATLCKSSQSVFYSSNQKNYLAWYQQKPG |
| | QAPRLLIYWASTRESGIPDRFSGSGSGTDFTLTISRLEPEDFAVYYCHQ |
| | YLSSLTFGQGTKVEIK (SEQ ID NO: 1432) |

Antibody 7E9

| | |
|---|---|
| 7E9V4-1 | DIVMTQSPDSLAVSLGERATINCKSSQSLLYSSNQKNCLAWYQQKPG |
| | QPPKLLIYWASTRESGVPDRFSGSGSGTDFTLTISSLQAEDVAVYYCQ |
| | QYYSYPLTFGQGTKVEIK (SEQ ID NO: 1434) |
| 7E9V2-28 | DIVMTQSPLSLPVTPGEPASISCKSSQSLLYSSNQKNCLAWYLQKPG |
| | QSPQLLIYWASTRESGVPDRFSGSGSGTDFTLKISRVEAEDVGVYYC |
| | QQYYSYPLTFGQGTKVEIK (SEQ ID NO: 1435) |
| 7E9V2-30 | DVVMTQSPLSLPVTLGQPASISCKSSQSLLYSSNQKNCLAWFQQRPG |
| | QSPRRLIYWASTRESGVPDRFSGSGSGTDFTLKISRVEAEDVGVYYC |
| | QQYYSYPLTFGQGTKVEIK (SEQ ID NO: 1436) |
| 7E9V1-9 | DIQLTQSPSFLSASVGDRVTITCKSSQSLLYSSNQKNCLAWYQQKPG |
| | KAPKLLIYWASTRESGVPSRFSGSGSGTEFTLTISSLQPEDFATYYCQ |
| | QYYSYPLTFGQGTKVEIK (SEQ ID NO: 1437) |
| 7E9V3-15 | EIVMTQSPATLSVSPGERATLSCKSSQSLLYSSNQKNCLAWYQQKPG |
| | QAPRLLIYWASTRESGIPARFSGSGSGTEFTLTISSLQSEDFAVYYCQQ |
| | YYSYPLTFGQGTKVEIK (SEQ ID NO: 1438) |
| 7E9V1-5 | DIQMTQSPSTLSASVGDRVTITCKSSQSLLYSSNQKNCLAWYQQKPG |
| | KAPKLLIYWASTRESGVPSRFSGSGSGTEFTLTISSLQPDDFATYYCQ |
| | QYYSYPLTFGQGTKVEIK (SEQ ID NO: 1439) |
| 7E9V1-33 | DIQMTQSPSSLSASVGDRVTITCKSSQSLLYSSNQKNCLAWYQQKPG |
| | KAPKLLIYWASTRESGVPSRFSGSGSGTDFTFTISSLQPEDIATYYCQQ |
| | YYSYPLTFGQGTKVEIK (SEQ ID NO: 1440) |
| 7E9V1-39 | DIQMTQSPSSLSASVGDRVTITCKSSQSLLYSSNQKNCLAWYQQKPG |
| | KAPKLLIYWASTRESGVPSRFSGSGSGTDFTLTISSLQPEDFATYYCQQ |
| | YYSYPLTFGQGTKVEIK (SEQ ID NO: 1441) |
| 7E9V3-11 | EIVLTQSPATLSLSPGERATLSCKSSQSLLYSSNQKNCLAWYQQKPG |
| | QAPRLLIYWASTRESGIPARFSGSGSGTDFTLTISSLEPEDFAVYYCQQ |
| | YYSYPLTFGQGTKVEIK (SEQ ID NO: 1442) |
| 7E9V3-20 | EIVLTQSPGTLSLSPGERATLSCKSSQSLLYSSNQKNCLAWYQQKPG |
| | QAPRLLIYWASTRESGIPDRFSGSGSGTDFTLTISRLEPEDFAVYYCQQ |
| | YYSYPLTFGQGTKVEIK (SEQ ID NO: 1443) |

Antibody 8C3

| | |
|---|---|
| 8C3V2-30 | DVVMTQSPLSLPVTLGQPASISCRSSQSLVHSNGNTYLHWFQQRPGQ |
| | SPRRLIYKVSNRFSGVPDRFSGSGSGTDFTLKISRVEAEDVGVYYCSQ |
| | STHVPPTFGQGTKVEIK (SEQ ID NO: 1445) |
| 8C3V2-28 | DIVMTQSPLSLPVTPGEPASISCRSSQSLVHSNGNTYLHWYLQKPGQSP |
| | QLLIYKVSNRFSGVPDRFSGSGSGTDFTLKISRVEAEDVGVYYCSQSTHV |
| | PPTFGQGTKVEIK (SEQ ID NO: 1446) |
| 8C3V4-1 | DIVMTQSPDSLAVSLGERATINCRSSQSLVHSNGNTYLHWYQQKPGQ |
| | PPKWYKVSNRFSGVPDRFSGSGSGTDFTLTISSLQAEDVAVYYCSQS |
| | THVPPTFGQGTKVEIK (SEQ ID NO: 1447) |
| 8C3V3-11 | EIVLTQSPATLSLSPGERATLSCRSSQSLVHSNGNTYLHWYQQKPGQ |
| | APRLLIYKVSNRFSGIPARFSGSGSGTDFTLTISSLEPEDFAVYYCSQST |
| | HVPPTFGQGTKVEIK (SEQ ID NO: 1448) |
| 8C3V1-33 | DIQMTQSPSSLSASVGDRVTITCRSSQSLVHSNGNTYLHWYQQKPGK |
| | APKLLIYKVSNRFSGVPSRFSGSGSGTDFTFTISSLQPEDIATYYCSQS |
| | THVPPTFGQGTKVEIK (SEQ ID NO: 1449) |
| 8C3V1-5 | DIQMTQSPSTLSASVGDRVTITCRSSQSLVHSNGNTYLHWYQQKPGK |
| | APKLLIYKVSNRFSGVPSRFSGSGSGTEFTLTISSLQPDDFATYYCSQST |
| | HVPPTFGQGTKVEIK (SEQ ID NO: 1450) |
| 8C3V1-39 | DIQMTQSPSSLSASVGDRVTITCRSSQSLVHSNGNTYLHWYQQKPGK |
| | APKWYKVSNRFSGVPSRFSGSGSGTDFTLTISSLQPEDFATYYCSQS |
| | THVPPTFGQGTKVEIK (SEQ ID NO: 1451) |
| 8C3V1-9 | DIQLTQSPSFLSASVGDRVTITCRSSQSLVHSNGNTYLHWYQQKPGK |
| | APKLLIYKVSNRFSGVPSRFSGSGSGTEFTLTISSLQPEDFATYYCSQS |
| | THVPPTFGQGTKVEIK (SEQ ID NO: 1452) |
| 8C3V3-15 | EIVMTQSPATLSVSPGERATLSCRSSQSLVHSNGNTYLHWYQQKPGQ |
| | APRLLIYKVSNRFSGIPARFSGSGSGTEFTLTISSLQSEDFAVYYCSQST |
| | HVPPTFGQGTKVEIK (SEQ ID NO: 1453) |
| 8C3V3-20 | EIVLTQSPGTLSLSPGERATLSCRSSQSLVHSNGNTYLHWYQQKPGQ |
| | APRLLIYKVSNRFSGIPDRFSGSGSGTDFTLTISRLEPEDFAVYYCSQST |
| | HVPPTFGQGTKVEIK (SEQ ID NO: 1454) |

TABLE D8
Humanized heavy chain variable region sequences

| | |
|---|---|
| Antibody variant | Humanized sequences |

Antibody 4D11

| | |
|---|---|
| 4D11V4-59 | QVQLQESGPGLVKPSETLSLTCTVSGFTLSSYAMSWIRQPPGKGLEW |
| | VASISRGGSTYYPPSLKSRVTISVDTSKNQFSLKLSSVTAADTAVYYC |
| | TRGYGYYRTPFANWGQGTLVTVSS (SEQ ID NO: 1220) |
| 4D11V3-23 | EVQLLESGGGLVQPGGSLRLSCAASGFTLSSYAMSWVRQAPGKGLE |
| | WVASISRGGSTYYPDSVKGRFTISRDNSKNTLYLQMNSLRAEDTAVY |
| | YCTRGYGYYRTPFANWGQGTLVTVSS (SEQ ID NO: 1221) |
| 4D11V3-7 | EVQLVESGGGLVQPGGSLRLSCAASGFTLSSYAMSWVRQAPGKGLE |
| | WVASISRGGSTYYPDSVKGRFTISRDNAKNSLYLQMNSLRAEDTAVY |
| | YCTRGYGYYRTPFANWGQGTLVTVSS (SEQ ID NO: 1222) |
| 4D11V3-48 | EVQLVESGGGLVQPGGSLRLSCAASGFTLSSYAMSWVRQAPGKGLE |
| | WVASISRGGSTYYPDSVKGRFTISRDNAKNSLYLQMNSLRAEDTAVY |
| | YCTRGYGYYRTPFANWGQGTLVTVSS (SEQ ID NO: 1222) |
| 4D11V3-30 | QVQLVESGGGVVQPGRSLRLSCAASGFTLSSYAMSWVRQAPGKGLE |
| | WVASISRGGSTYYPDSVKGRFTISRDNSKNTLYLQMNSLRAEDTAVY |
| | YCTRGYGYYRTPFANWGQGTLVTVSS (SEQ ID NO: 1223) |
| 4D11V1-69 | QVQLVQSGAEVKKPGSSVKVSCKASGFTLSSYAMSWVRQAPGQGLE |
| | WVASISRGGSTYYPQKFQGRVTITADESTSTAYMELSSLRSEDTAVYY |
| | CTRGYGYYRTPFANWGQGTLVTVSS (SEQ ID NO: 1224) |
| 4D11V1-46 | QVQLVQSGAEVKKPGASVKVSCKASGFTLSSYAMSWVRQAPGQGLE |
| | WVASISRGGSTYYPQKFQGRVTMTRDTSTSTVYMELSSLRSEDTAVY |
| | YCTRGYGYYRTPFANWGQGTLVTVSS (SEQ ID NO: 1225) |
| 4D11V5-51 | EVQLVQSGAEVKKPGESLKISCKGSGFTLSSYAMSWVRQMPGKGLE |
| | WVASISRGGSTYYPPSFQGQVTISADKSISTAYLQWSSLKASDTAMYY |
| | CTRGYGYYRTPFANWGQGTLVTVSS (SEQ ID NO: 1226) |
| 4D11V4-39 | QLQLQESGPGLVKPSETLSLTCTVSGFTLSSYAMSWIRQPPGKGLEW |
| | VASISRGGSTYYPPSLKSRVTISVDTSKNQFSLKLSSVTAADTAVYYC |
| | TRGYGYYRTPFANWGQGTLVTVSS (SEQ ID NO: 1227) |
| 4D11V4-30-4 | QVQLQESGPGLVKPSQTLSLTCTVSGFTLSSYAMSWIRQPPGKGLEW |
| | VASISRGGSTYYPPSLKSRVTISVDTSKNQFSLKLSSVTAADTAVYYC |
| | TRGYGYYRTPFANWGQGTLVTVSS (SEQ ID NO: 1228) |

Antibody 7C5

| | |
|---|---|
| 7C5V4-59 | QVQLQESGPGLVKPSETLSLTCTVSGFTLSSYAMSWIRQPPGKGLEW |
| | VASISRGGSTYYPPSLKSRVTISVDTSKNQFSLKLSSVTAADTAVYYC |
| | TRGYGYYRTPFANWGQGTLVTVSS (SEQ ID NO: 1220) |
| 7C5V3-23 | EVQLLESGGGLVQPGGSLRLSCAASGFTLSSYAMSWVRQAPGKGLE |
| | WVASISRGGSTYYPDSVKGRFTISRDNSKNTLYLQMNSLRAEDTAVY |
| | YCTRGYGYYRTPFANWGQGTLVTVSS (SEQ ID NO: 1221) |
| 7C5V3-7 | EVQLVESGGGLVQPGGSLRLSCAASGFTLSSYAMSWVRQAPGKGLE |
| | WVASISRGGSTYYPDSVKGRFTISRDNAKNSLYLQMNSLRAEDTAV |
| | YYCTRGYGYYRTPFANWGQGTLVTVSS (SEQ ID NO: 1222) |
| 7C5V3-48 | EVQLVESGGGLVQPGGSLRLSCAASGFTLSSYAMSWVRQAPGKGLE |
| | WVASISRGGSTYYPDSVKGRFTISRDNAKNSLYLQMNSLRAEDTAV |
| | YYCTRGYGYYRTPFANWGQGTLVTVSS (SEQ ID NO: 1222) |
| 7C5V3-30 | QVQLVESGGGVVQPGRSLRLSCAASGFTLSSYAMSWVRQAPGKGLE |
| | WVASISRGGSTYYPDSVKGRFTISRDNSKNTLYLQMNSLRAEDTAVY |
| | YCTRGYGYYRTPFANWGQGTLVTVSS (SEQ ID NO: 1223) |
| 7C5V1-69 | QVQLVQSGAEVKKPGSSVKVSCKASGFTLSSYAMSWVRQAPGQGLE |
| | WVASISRGGSTYYPQKFQGRVTITADESTSTAYMELSSLRSEDTAVY |
| | YCTRGYGYYRTPFANWGQGTLVTVSS (SEQ ID NO: 1224) |
| 7C5V1-46 | QVQLVQSGAEVKKPGASVKVSCKASGFTLSSYAMSWVRQAPGQGL |
| | EWVASISRGGSTYYPQKFQGRVTMTRDTSTSTVYMELSSLRSEDTAV |
| | YYCTRGYGYYRTPFANWGQGTLVTVSS (SEQ ID NO: 1225) |
| 7C5V5-51 | EVQLVQSGAEVKKPGESLKISCKGSGFTLSSYAMSWVRQMPGKGLE |
| | WVASISRGGSTYYPPSFQGQVTISADKSISTAYLQWSSLKASDTAMY |
| | YCTRGYGYYRTPFANWGQGTLVTVSS (SEQ ID NO: 1226) |
| 7C5V4-39 | QLQLQESGPGLVKPSETLSLTCTVSGFTLSSYAMSWIRQPPGKGLEW |
| | VASISRGGSTYYPPSLKSRVTISVDTSKNQFSLKLSSVTAADTAVYYC |
| | TRGYGYYRTPFANWGQGTLVTVSS (SEQ ID NO: 1227) |
| 7C5V4-30-4 | QVQLQESGPGLVKPSQTLSLTCTVSGFTLSSYAMSWIRQPPGKGLEW |
| | VASISRGGSTYYPPSLKSRVTISVDTSKNQFSLKLSSVTAADTAVYYC |
| | TRGYGYYRTPFANWGQGTLVTVSS (SEQ ID NO: 1228) |

Antibody 6G12

| | |
|---|---|
| 6G12V1-46 | QVQLVQSGAEVKKPGASVKVSCKASGYTFTEYTMHWVRQAPGQGL |
| | EWIGGINPNNGGTSYSQKFQGRVTMTRDTSTSTVYMELSSLRSEDTA |
| | VYYCARGGSHYYAMDYWGQGTLVTVSS (SEQ ID NO: 1230) |
| 6G12V5-51 | EVQLVQSGAEVKKPGESLKISCKGSGYTFTEYTMHWVRQMPGKGLE |
| | WIGGINPNNGGTSYSPSFQGQVTISADKSISTAYLQWSSLKASDTAMY |
| | YCARGGSHYYAMDYWGQGTLVTVSS (SEQ ID NO: 1231) |
| 6G12V1-69 | QVQLVQSGAEVKKPGSSVKVSCKASGYTFTEYTMEIWVRQAPGQGL |
| | EWIGGINPNNGGTSYSQKFQGRVTITADESTSTAYMELSSLRSEDTAV |
| | YYCARGGSHYYAMDYWGQGTLVTVSS (SEQ ID NO: 1232) |
| 6G12V3-23 | EVQLLESGGGLVQPGGSLRLSCAASGYTFTEYTMHWVRQAPGKGLE |
| | WIGGINPNNGGTSYSDSVKGRFTISRDNSKNTLYLQMNSLRAEDTAV |
| | YYCARGGSHYYAMDYWGQGTLVTVSS (SEQ ID NO: 1233) |
| 6G12V3-30 | QVQLVESGGGVVQPGRSLRLSCAASGYTFTEYTMEIWVRQAPGKGL |
| | EWIGGINPNNGGTSYSDSVKGRFTISRDNSKNTLYLQMNSLRAEDTA |
| | VYYCARGGSHYYAMDYWGQGTLVTVSS (SEQ ID NO: 1234) |
| 6G12V3-48 | EVQLVESGGGLVQPGGSLRLSCAASGYTFTEYTMHWVRQAPGKGLE |
| | WIGGINPNNGGTSYSDSVKGRFTISRDNAKNSLYLQMNSLRAEDTAV |
| | YYCARGGSHYYAMDYWGQGTLVTVSS (SEQ ID NO: 1235) |
| 6G12V3-7 | EVQLVESGGGLVQPGGSLRLSCAASGYTFTEYTMHWVRQAPGKGLE |
| | WIGGINPNNGGTSYSDSVKGRFTISRDNAKNSLYLQMNSLRAEDTAV |
| | YYCARGGSHYYAMDYWGQGTLVTVSS (SEQ ID NO: 1235) |
| 6G12V4-59 | QVQLQESGPGLVKPSETLSLTCTVSGYTFTEYTMHWIRQPPGKGLEW |
| | IGGINPNNGGTSYSPSLKSRVTISVDTSKNQFSLKLSSVTAADTAVYY |
| | CARGGSHYYAMDYWGQGTLVTVSS (SEQ ID NO: 1236) |
| 6G12V3-15 | EVQLVESGGGLVKPGGSLRLSCAASGYTFTEYTMHWVRQAPGKGLEWI |
| | GGINPNNGGTSYSAPVKGRFTISRDDSKNTLYLQMNSLKTEDTAVYYCA |
| | RGGSHYYAMDYWGQGTLVTVSS (SEQ ID NO: 1237) |
| 6G12V4-39 | QLQLQESGPGLVKPSETLSLTCTVSGYTFTEYTMHWIRQPPGKGLEWIGG |
| | INPNNGGTSYSPSLKSRVTISVDTSKNQFSLKLSSVTAADTAVYYCARGG |
| | SHYYAMDYWGQGTLVTVSS (SEQ ID NO: 1238) |

Antibody 8E10

| | |
|---|---|
| 8E10V1-46 | QVQLVQSGAEVKKPGASVKVSCKASGYTFTDYEMHWVRQAPGQGLEWI |
| | GVIDPETGGTAYNQKFQGRVTMTRDTSTSTVYMELSSLRSEDTAVYYCTS |
| | PDYYGSSYPLYYAMDYWGQGTLVTVSS (SEQ ID NO: 1240) |
| 8E10V5-51 | EVQLVQSGAEVKKPGESLKISCKGSGYTFTDYEMEIWVRQMPGKGLEWIG |
| | VIDPETGGTAYNPSFQGQVTISADKSISTAYLQWSSLKASDTAMYYCTSPD |
| | YYGSSYPLYYAMDYWGQGTLVTVSS (SEQ ID NO: 1241) |
| 8E10V3-30 | QVQLVESGGGVVQPGRSLRLSCAASGYTFTDYEMEIWVRQAPGKGLEWIG |
| | VIDPETGGTAYNDSVKGRFTISRDNSKNTLYLQMNSLRAEDTAVYYCTSP |
| | DYYGSSYPLYYAMDYWGQGTLVTVSS (SEQ ID NO: 1242) |
| 8E10V3-23 | EVQLLESGGGLVQPGGSLRLSCAASGYTFTDYEMEIWVRQAPGKGLEWIG |
| | VIDPETGGTAYNDSVKGRFTISRDNSKNTLYLQMNSLRAEDTAVYYCTSP |
| | DYYGSSYPLYYAMDYWGQGTLVTVSS (SEQ ID NO: 1243) |
| 8E10V1-69 | QVQLVQSGAEVKKPGSSVKVSCKASGYTFTDYEMEIWVRQAPGQGLEWI |
| | GVIDPETGGTAYNQKFQGRVTITADESTSTAYMELSSLRSEDTAVYYCTSP |
| | DYYGSSYPLYYAMDYWGQGTLVTVSS (SEQ ID NO: 1244) |
| 8E10V3-48 | EVQLVESGGGLVQPGGSLRLSCAASGYTFTDYENIHWVRQAPGKGLEWIG |
| | VIDPETGGTAYNDSVKGRFTISRDNAKNSLYLQMNSLRAEDTAVYYCTSP |
| | DYYGSSYPLYYAMDYWGQGTLVTVSS (SEQ ID NO: 1245) |
| 8E10V3-7 | EVQLVESGGGLVQPGGSLRLSCAASGYTFTDYENIHWVRQAPGKGLEWIG |
| | VIDPETGGTAYNDSVKGRFTISRDNAKNSLYLQMNSLRAEDTAVYYCTSP |
| | DYYGSSYPLYYAMDYWGQGTLVTVSS (SEQ ID NO: 1245) |
| 8E10V4-59 | QVQLQESGPGLVKPSETLSLTCTVSGYTFTDYENIHWIRQPPGKGLEWIGVI |
| | DPETGGTAYNPSLKSRVTISVDTSKNQFSLKLSSVTAADTAVYYCTSPDYY |
| | GSSYPLYYAMDYWGQGTLVTVSS (SEQ ID NO: 1246) |
| 8E10V3-15 | EVQLVESGGGLVKPGGSLRLSCAASGYTFTDYENIHWVRQAPGKGLEWIG |
| | VIDPETGGTAYNAPVKGRFTISRDDSKNTLYLQMNSLKTEDTAVYYCTSP |
| | DYYGSSYPLYYAMDYWGQGTLVTVSS (SEQ ID NO: 1247) |
| 8E10V4-39 | QLQLQESGPGLVKPSETLSLTCTVSGYTFTDYENIHWIRQPPGKGLEWIGVI |
| | DPETGGTAYNPSLKSRVTISVDTSKNQFSLKLSSVTAADTAVYYCTSPDYY |
| | GSSYPLYYAMDYWGQGTLVTVSS (SEQ ID NO: 1248) |

Antibody 7E5

| | |
|---|---|
| 7E5V3-15 | EVQLVESGGGLVKPGGSLRLSCAASGFTFSDAWMGWVRQAPGKGLEWV |
| | AEIRDKVKNHATYYAAPVKGRFTISRDDSKNTLYLQMNSLKTEDTAVYY |
| | CRLGVFDYWGQGTLVTVSS (SEQ ID NO: 1250) |
| 7E5V3-7 | EVQLVESGGGLVQPGGSLRLSCAASGFTFSDAWMGWVRQAPGKGLEWV |
| | AEIRDKVKNHATYYADSVKGRFTISRDNAKNSLYLQMNSLRAEDTAVYY |
| | CRLGVFDYWGQGTLVTVSS (SEQ ID NO: 1251) |
| 7E5V3-23 | EVQLLESGGGLVQPGGSLRLSCAASGFTFSDAWMGWVRQAPGKGLEWV |
| | AEIRDKVKNHATYYADSVKGRFTISRDNSKNTLYLQMNSLRAEDTAVYY |
| | CRLGVFDYWGQGTLVTVSS (SEQ ID NO: 1252) |
| 7E5V3-48 | EVQLVESGGGLVQPGGSLRLSCAASGFTFSDAWMGWVRQAPGKGLEWV |
| | AEIRDKVKNHATYYADSVKGRFTISRDNAKNSLYLQMNSLRAEDTAVYY |
| | CRLGVFDYWGQGTLVTVSS (SEQ ID NO: 1251) |
| 7E5V3-30 | QVQLVESGGGVVQPGRSLRLSCAASGFTFSDAWMGWVRQAPGKGLEWV |
| | AEIRDKVKNHATYYADSVKGRFTISRDNSKNTLYLQMNSLRAEDTAVYY |
| | CRLGVFDYWGQGTLVTVSS (SEQ ID NO: 1253) |
| 7E5V1-69 | QVQLVQSGAEVKKPGSSVKVSCKASGFTFSDAWMGWVRQAPGQGLEWV |
| | AEIRDKVKNHATYYAQKFQGRVTITADESTSTAYMELSSLRSEDTAVYYC |
| | RLGVFDYWGQGTLVTVSS (SEQ ID NO: 1254) |
| 7E5V1-46 | QVQLVQSGAEVKKPGASVKVSCKASGFTFSDAWMGWVRQAPGQGLEW |
| | VAEIRDKVKNHATYYAQKFQGRVTMTRDTSTSTVYMELSSLRSEDTAVY |
| | YCRLGVFDYWGQGTLVTVSS (SEQ ID NO: 1255) |
| 7E5V5-51 | EVQLVQSGAEVKKPGESLKISCKGSGFTFSDAWMGWVRQMPGKGLEWV |
| | AEIRDKVKNHATYYAPSFQGQVTISADKSISTAYLQWSSLKASDTAMYYC |
| | RLGVFDYWGQGTLVTVSS (SEQ ID NO: 1256) |
| 7E5V4-59 | QVQLQESGPGLVKPSETLSLTCTVSGFTFSDAWMGWIRQPPGKGLEWVAE |
| | IRDKVKNHATYYAPSLKSRVTISVDTSKNQFSLKLSSVTAADTAVYYCRL |
| | GVFDYWGQGTLVTVSS (SEQ ID NO: 1257) |
| 7E5V4-39 | QLQLQESGPGLVKPSETLSLTCTVSGFTFSDAWMGWIRQPPGKGLEWVAE |
| | IRDKVKNHATYYAPSLKSRVTISVDTSKNQFSLKLSSVTAADTAVYYCRL |
| | GVFDYWGQGTLVTVSS (SEQ ID NO: 1258) |

Antibody 7F8

| | |
|---|---|
| 7F8V3-15 | EVQLVESGGGLVKPGGSLRLSCAASGFSFNTYAMNWVRQAPGKGLEWIA |
| | RIRSKSNNYATYYAAPVKGRFTISRDDSKNTLYLQMNSLKTEDTAVYYCV |
| | RHGDGNLWYIDVWGQGTLVTVSS (SEQ ID NO: 1260) |
| 7F8V3-48 | EVQLVESGGGLVQPGGSLRLSCAASGFSFNTYAMNWVRQAPGKGLEWIA |
| | RIRSKSNNYATYYADSVKGRFTISRDNAKNSLYLQMNSLRAEDTAVYYCV |
| | RHGDGNLWYIDVWGQGTLVTVSS (SEQ ID NO: 1261) |
| 7F8V3-23 | EVQLLESGGGLVQPGGSLRLSCAASGFSFNTYAMNWVRQAPGKGLEWIA |
| | RIRSKSNNYATYYADSVKGRFTISRDNSKNTLYLQMNSLRAEDTAVYYCV |
| | RHGDGNLWYIDVWGQGTLVTVSS (SEQ ID NO: 1262) |
| 7F8V3-7 | EVQLVESGGGLVQPGGSLRLSCAASGFSFNTYAMNWVRQAPGKGLEWIA |
| | RIRSKSNNYATYYADSVKGRFTISRDNAKNSLYLQMNSLRAEDTAVYYCV |
| | RHGDGNLWYIDVWGQGTLVTVSS (SEQ ID NO: 1261) |
| 7F8V3-30 | QVQLVESGGGVVQPGRSLRLSCAASGFSFNTYAMNWVRQAPGKGLEWIA |
| | RIRSKSNNYATYYADSVKGRFTISRDNSKNTLYLQMNSLRAEDTAVYYCV |
| | RHGDGNLWYIDVWGQGTLVTVSS (SEQ ID NO: 1263) |
| 7F8V1-69 | QVQLVQSGAEVKKPGSSVKVSCKASGFSFNTYAMNWVRQAPGQGLEWI |
| | ARIRSKSNNYATYYAQKFQGRVTITADESTSTAYMELSSLRSEDTAVYYC |
| | VRHGDGNLWYIDVWGQGTLVTVSS (SEQ ID NO: 1264) |
| 7F8V5-51 | EVQLVQSGAEVKKPGESLKISCKGSGFSFNTYAMNWVRQMPGKGLEWIA |
| | RIRSKSNNYATYYAPSFQGQVTISADKSISTAYLQWSSLKASDTAMYYCV |
| | RHGDGNLWYIDVWGQGTLVTVSS (SEQ ID NO: 1265) |
| 7F8V1-46 | QVQLVQSGAEVKKPGASVKVSCKASGFSFNTYAMNWVRQAPGQGLEWI |
| | ARIRSKSNNYATYYAQKFQGRVTMTRDTSTSTVYMELSSLRSEDTAVYYC |
| | VRHGDGNLWYIDVWGQGTLVTVSS (SEQ ID NO: 1266) |
| 7F8V4-59 | QVQLQESGPGLVKPSETLSLTCTVSGFSFNTYAMNWIRQPPGKGLEWIARI |
| | RSKSNNYATYYAPSLKSRVTISVDTSKNQFSLKLSSVTAADTAVYYCVRH |
| | GDGNLWYIDVWGQGTLVTVSS (SEQ ID NO: 1267) |
| 7F8V4-30-4 | QVQLQESGPGLVKPSQTLSLTCTVSGFSFNTYAMNWIRQPPGKGLEWIARI |
| | RSKSNNYATYYAPSLKSRVTISVDTSKNQFSLKLSSVTAADTAVYYCVRH |
| | GDGNLWYIDVWGQGTLVTVSS (SEQ ID NO: 1268) |

Antibody 8F8

| | |
|---|---|
| 8F8V1-46 | QVQLVQSGAEVKKPGASVKVSCKASGYTVSRYWMIHWVRQAPGQGLEWI |
| | GRIDPNSGGTKYNQKFQGRVTMTRDTSTSTVYMELSSLRSEDTAVYYCVL |
| | TGTDFDYWGQGTLVTVSS (SEQ ID NO: 1270) |
| 8F8V3-23 | EVQLLESGGGLVQPGGSLRLSCAASGYTVSRYWMHWVRQAPGKGLEWI |
| | GRIDPNSGGTKYNDSVKGRFTISRDNSKNTLYLQMNSLRAEDTAVYYCVL |
| | TGTDFDYWGQGTLVTVSS (SEQ ID NO: 1271) |
| 8F8V1-69 | QVQLVQSGAEVKKPGSSVKVSCKASGYTVSRYWMHWVRQAPGQGLEWI |
| | GRIDPNSGGTKYNQKFQGRVTITADESTSTAYMELSSLRSEDTAVYYCVLT |
| | GTDFDYWGQGTLVTVSS (SEQ ID NO: 1272) |
| 8F8V5-51 | EVQLVQSGAEVKKPGESLKISCKGSGYTVSRYWMHWVRQMPGKGLEWI |
| | GRIDPNSGGTKYNPSFQGQVTISADKSISTAYLQWSSLKASDTAMYYCVLT |
| | GTDFDYWGQGTLVTVSS (SEQ ID NO: 1273) |
| 8F8V3-48 | EVQLVESGGGLVQPGGSLRLSCAASGYTVSRYWMHWVRQAPGKGLEWI |
| | GRIDPNSGGTKYNDSVKGRFTISRDNAKNSLYLQMNSLRAEDTAVYYCVL |
| | TGTDFDYWGQGTLVTVSS (SEQ ID NO: 1274) |
| 8F8V3-30 | QVQLVESGGGVVQPGRSLRLSCAASGYTVSRYWMHWVRQAPGKGLEWI |
| | GRIDPNSGGTKYNDSVKGRFTISRDNSKNTLYLQMNSLRAEDTAVYYCVL |
| | TGTDFDYWGQGTLVTVSS (SEQ ID NO: 1275) |
| 8F8V3-7 | EVQLVESGGGLVQPGGSLRLSCAASGYTVSRYWMHWVRQAPGKGLEWI |
| | GRIDPNSGGTKYNDSVKGRFTISRDNAKNSLYLQMNSLRAEDTAVYYCVL |
| | TGTDFDYWGQGTLVTVSS (SEQ ID NO: 1274) |
| 8F8V4-59 | QVQLQESGPGLVKPSETLSLTCTVSGYTVSRYWMHWIRQPPGKGLEWIGR |
| | IDPNSGGTKYNPSLKSRVTISVDTSKNQFSLKLSSVTAADTAVYYCVLTGT |
| | DFDYWGQGTLVTVSS (SEQ ID NO: 1276) |
| 8F8V3-15 | EVQLVESGGGLVKPGGSLRLSCAASGYTVSRYWMHWVRQAPGKGLEWI |
| | GRIDPNSGGTKYNAPVKGRFTISRDDSKNTLYLQMNSLKTEDTAVYYCVL |
| | TGTDFDYWGQGTLVTVSS (SEQ ID NO: 1277) |
| 8F8V4-30-4 | QVQLQESGPGLVKPSQTLSLTCTVSGYTVSRYWNIHWIRQPPGKGLEWIGR |
| | IDPNSGGTKYNPSLKSRVTISVDTSKNQFSLKLSSVTAADTAVYYCVLTGT |
| | DFDYWGQGTLVTVSS (SEQ ID NO: 1278) |

Antibody1H7

| | |
|---|---|
| 1H7V3-15 | EVQLVESGGGLVKPGGSLRLSCAASGFSFNTYAMNWVRQAPGKGLEWIA |
| | RIRSKSNNYATYYAAPVKGRFTISRDDSKNTLYLQMNSLKTEDTAVYYCV |
| | RHGDGNLWYIDVWGQGTLVTVSS (SEQ ID NO: 1260) |
| 1H7V3-23 | EVQLLESGGGLVQPGGSLRLSCAASGFSFNTYAMNWVRQAPGKGLEWIA |
| | RIRSKSNNYATYYADSVKGRFTISRDNSKNTLYLQMNSLRAEDTAVYYCV |
| | RHGDGNLWYIDVWGQGTLVTVSS (SEQ ID NO: 1262) |
| 1H7V3-48 | EVQLVESGGGLVQPGGSLRLSCAASGFSFNTYAMNWVRQAPGKGLEWIA |
| | RIRSKSNNYATYYADSVKGRFTISRDNAKNSLYLQMNSLRAEDTAVYYCV |
| | RHGDGNLWYIDVWGQGTLVTVSS (SEQ ID NO: 1261) |
| 1H7V3-7 | EVQLVESGGGLVQPGGSLRLSCAASGFSFNTYAMNWVRQAPGKGLEWIA |
| | RIRSKSNNYATYYADSVKGRFTISRDNAKNSLYLQMNSLRAEDTAVYYCV |
| | RHGDGNLWYIDVWGQGTLVTVSS (SEQ ID NO: 1261) |
| 1H7V3-30 | QVQLVESGGGVVQPGRSLRLSCAASGFSFNTYAMNWVRQAPGKGLEWIA |
| | RIRSKSNNYATYYADSVKGRFTISRDNSKNTLYLQMNSLRAEDTAVYYCV |
| | RHGDGNLWYIDVWGQGTLVTVSS (SEQ ID NO: 1263) |
| 1H7V5-51 | EVQLVQSGAEVKKPGESLKISCKGSGFSFNTYAMNWVRQMPGKGLEWIA |
| | RIRSKSNNYATYYAPSFQGQVTISADKSISTAYLQWSSLKASDTAMYYCV |
| | RHGDGNLWYIDVWGQGTLVTVSS (SEQ ID NO: 1265) |
| 1H7V1-69 | QVQLVQSGAEVKKPGSSVKVSCKASGFSFNTYAMNWVRQAPGQGLEWI |
| | ARIRSKSNNYATYYAQKFQGRVTITADESTSTAYMELSSLRSEDTAVYYC |
| | VRHGDGNLWYIDVWGQGTLVTVSS (SEQ ID NO: 1264) |
| 1H7V1-46 | QVQLVQSGAEVKKPGASVKVSCKASGFSFNTYAMNWVRQAPGQGLEWI |
| | ARIRSKSNNYATYYAQKFQGRVTMTRDTSTSTVYMELSSLRSEDTAVYYC |
| | VRHGDGNLWYIDVWGQGTLVTVSS (SEQ ID NO: 1266) |
| 1H7V4-59 | QVQLQESGPGLVKPSETLSLTCTVSGFSFNTYAMNWIRQPPGKGLEWIARI |
| | RSKSNNYATYYAPSLKSRVTISVDTSKNQFSLKLSSVTAADTAVYYCVRH |
| | GDGNLWYIDVWGQGTLVTVSS (SEQ ID NO: 1267) |
| 1H7V4-30-4 | QVQLQESGPGLVKPSQTLSLTCTVSGFSFNTYAMNWIRQPPGKGLEWIARI |
| | RSKSNNYATYYAPSLKSRVTISVDTSKNQFSLKLSSVTAADTAVYYCVRH |
| | GDGNLWYIDVWGQGTLVTVSS (SEQ ID NO: 1268) |

Antibody 2H8

| | |
|---|---|
| 2H8V3-15 | EVQLVESGGGLVKPGGSLRLSCAASGFSFNTYAMNWVRQAPGKGLEWIA |
| | RIRSKSNNYATYYAAPVKGRFTISRDDSKNTLYLQMNSLKTEDTAVYYCV |
| | RHGDGNLWYIDVWGQGTLVTVSS (SEQ ID NO: 1260) |
| 2H8V3-48 | EVQLVESGGGLVQPGGSLRLSCAASGFSFNTYAMNWVRQAPGKGLEWIA |
| | RIRSKSNNYATYYADSVKGRFTISRDNAKNSLYLQMNSLRAEDTAVYYCV |
| | RHGDGNLWYIDVWGQGTLVTVSS (SEQ ID NO: 1261) |
| 2H8V3-23 | EVQLLESGGGLVQPGGSLRLSCAASGFSFNTYAMNWVRQAPGKGLEWIA |
| | RIRSKSNNYATYYADSVKGRFTISRDNSKNTLYLQMNSLRAEDTAVYYCV |
| | RHGDGNLWYIDVWGQGTLVTVSS (SEQ ID NO: 1262) |
| 2H8V3-7 | EVQLVESGGGLVQPGGSLRLSCAASGFSFNTYAMNWVRQAPGKGLEWIA |
| | RIRSKSNNYATYYADSVKGRFTISRDNAKNSLYLQMNSLRAEDTAVYYCV |
| | RHGDGNLWYIDVWGQGTLVTVSS (SEQ ID NO: 1261) |
| 2H8V3-30 | QVQLVESGGGVVQPGRSLRLSCAASGFSFNTYAMNWVRQAPGKGLEWIA |
| | RIRSKSNNYATYYADSVKGRFTISRDNSKNTLYLQMNSLRAEDTAVYYCV |
| | RHGDGNLWYIDVWGQGTLVTVSS (SEQ ID NO: 1263) |
| 2H8V5-51 | EVQLVQSGAEVKKPGESLKISCKGSGFSFNTYAMNWVRQMPGKGLEWIA |
| | RIRSKSNNYATYYAPSFQGQVTISADKSISTAYLQWSSLKASDTAMYYCV |
| | RHGDGNLWYIDVWGQGTLVTVSS (SEQ ID NO: 1265) |
| 2H8V1-69 | QVQLVQSGAEVKKPGSSVKVSCKASGFSFNTYAMNWVRQAPGQGLEWI |
| | ARIRSKSNNYATYYAQKFQGRVTITADESTSTAYMELSSLRSEDTAVYYC |
| | VRHGDGNLWYIDVWGQGTLVTVSS (SEQ ID NO: 1264) |
| 2H8V1-46 | QVQLVQSGAEVKKPGASVKVSCKASGFSFNTYAMNWVRQAPGQGLEWI |
| | ARIRSKSNNYATYYAQKFQGRVTMTRDTSTSTVYMELSSLRSEDTAVYYC |
| | VRHGDGNLWYIDVWGQGTLVTVSS (SEQ ID NO: 1266) |
| 2H8V4-59 | QVQLQESGPGLVKPSETLSLTCTVSGFSFNTYAMNWIRQPPGKGLEWIARI |
| | RSKSNNYATYYAPSLKSRVTISVDTSKNQFSLKLSSVTAADTAVYYCVRH |
| | GDGNLWYIDVWGQGTLVTVSS (SEQ ID NO: 1267) |
| 2H8V4-30-4 | QVQLQESGPGLVKPSQTLSLTCTVSGFSFNTYAMNWIRQPPGKGLEWIARI |
| | RSKSNNYATYYAPSLKSRVTISVDTSKNQFSLKLSSVTAADTAVYYCVRH |
| | GDGNLWYIDVWGQGTLVTVSS (SEQ ID NO: 1268) |

Antibody 3A2

| | |
|---|---|
| 3A2V5-51 | EVQLVQSGAEVKKPGESLKISCKGSGYPFSNFWITWVRQMPGKGLEWIGD |
| | IYPGSDNSNYNPSFQGQVTISADKSISTAYLQWSSLKASDTAMYYCAREAY |
| | YTNPGFAYWGQGTLVTVSS (SEQ ID NO: 1282) |
| 3A2V1-69 | QVQLVQSGAEVKKPGSSVKVSCKASGYPFSNFWITWVRQAPGQGLEWIG |
| | DIYPGSDNSNYNQKFQGRVTITADESTSTAYMELSSLRSEDTAVYYCAREA |
| | YYTNPGFAYWGQGTLVTVSS (SEQ ID NO: 1283) |
| 3A2V1-46 | QVQLVQSGAEVKKPGASVKVSCKASGYPFSNFWITWVRQAPGQGLEWIG |
| | DIYPGSDNSNYNQKFQGRVTMTRDTSTSTVYMELSSLRSEDTAVYYCARE |
| | AYYTNPGFAYWGQGTLVTVSS (SEQ ID NO: 1284) |
| 3A2V3-48 | EVQLVESGGGLVQPGGSLRLSCAASGYPFSNFWITWVRQAPGKGLEWIGD |
| | IYPGSDNSNYNDSVKGRFTISRDNAKNSLYLQMNSLRAEDTAVYYCAREA |
| | YYTNPGFAYWGQGTLVTVSS (SEQ ID NO: 1285) |
| 3A2V3-30 | QVQLVESGGGVVQPGRSLRLSCAASGYPFSNFWITWVRQAPGKGLEWIG |
| | DIYPGSDNSNYNDSVKGRFTISRDNSKNTLYLQMNSLRAEDTAVYYCARE |
| | AYYTNPGFAYWGQGTLVTVSS (SEQ ID NO: 1286) |
| 3A2V3-7 | EVQLVESGGGLVQPGGSLRLSCAASGYPFSNFWITWVRQAPGKGLEWIGD |
| | IYPGSDNSNYNDSVKGRFTISRDNAKNSLYLQMNSLRAEDTAVYYCAREA |
| | YYTNPGFAYWGQGTLVTVSS (SEQ ID NO: 1285) |
| 3A2V3-23 | EVQLLESGGGLVQPGGSLRLSCAASGYPFSNFWITWVRQAPGKGLEWIGD |
| | IYPGSDNSNYNDSVKGRFTISRDNSKNTLYLQMNSLRAEDTAVYYCAREA |
| | YYTNPGFAYWGQGTLVTVSS (SEQ ID NO: 1287) |
| 3A2V4-59 | QVQLQESGPGLVKPSETLSLTCTVSGYPFSNFWITWIRQPPGKGLEWIGDIY |
| | PGSDNSNYNPSLKSRVTISVDTSKNQFSLKLSSVTAADTAVYYCAREAYY |
| | TNPGFAYWGQGTLVTVSS (SEQ ID NO: 1288) |
| IGHV3-15 | EVQLVESGGGLVKPGGSLRLSCAASGYPFSNFWITWVRQAPGKGLEWIGD |
| | IYPGSDNSNYNAPVKGRFTISRDDSKNTLYLQMNSLKTEDTAVYYCAREA |
| | YYTNPGFAYWGQGTLVTVSS (SEQ ID NO: 1289) |
| 3A2V4-39 | QLQLQESGPGLVKPSETLSLTCTVSGYPFSNFWITWIRQPPGKGLEWIGDIY |
| | PGSDNSNYNPSLKSRVTISVDTSKNQFSLKLSSVTAADTAVYYCAREAYY |
| | TNPGFAYWGQGTLVTVSS (SEQ ID NO: 1290) |

Antibody 3A7

| | |
|---|---|
| 3A7V3-15 | EVQLVESGGGLVKPGGSLRLSCAASGFTFSDAWMGWVRQAPGKGLEWV |
| | AEIRDKVKNHATYYAAPVKGRFTISRDDSKNTLYLQMNSLKTEDTAVYY |
| | CRLGVFDYWGQGTLVTVSS (SEQ ID NO: 1250) |
| 3A7V3-7 | EVQLVESGGGLVQPGGSLRLSCAASGFTFSDAWMGWVRQAPGKGLEWV |
| | AEIRDKVKNHATYYADSVKGRFTISRDNAKNSLYLQMNSLRAEDTAVYY |
| | CRLGVFDYWGQGTLVTVSS (SEQ ID NO: 1251) |
| 3A7V3-23 | EVQLLESGGGLVQPGGSLRLSCAASGFTFSDAWMGWVRQAPGKGLEWV |
| | AEIRDKVKNHATYYADSVKGRFTISRDNSKNTLYLQMNSLRAEDTAVYY |
| | CRLGVFDYWGQGTLVTVSS (SEQ ID NO: 1252) |
| 3A7V3-48 | EVQLVESGGGLVQPGGSLRLSCAASGFTFSDAWMGWVRQAPGKGLEWV |
| | AEIRDKVKNHATYYADSVKGRFTISRDNAKNSLYLQMNSLRAEDTAVYY |
| | CRLGVFDYWGQGTLVTVSS (SEQ ID NO: 1251) |
| 3A7V3-30 | QVQLVESGGGVVQPGRSLRLSCAASGFTFSDAWMGWVRQAPGKGLEWV |
| | AEIRDKVKNHATYYADSVKGRFTISRDNSKNTLYLQMNSLRAEDTAVYY |
| | CRLGVFDYWGQGTLVTVSS (SEQ ID NO: 1253) |
| 3A7V1-69 | QVQLVQSGAEVKKPGSSVKVSCKASGFTFSDAWMGWVRQAPGQGLEWV |
| | AEIRDKVKNHATYYAQKFQGRVTITADESTSTAYMELSSLRSEDTAVYYC |
| | RLGVFDYWGQGTLVTVSS (SEQ ID NO: 1254) |
| 3A7V1-46 | QVQLVQSGAEVKKPGASVKVSCKASGFTFSDAWMGWVRQAPGQGLEW |
| | VAEIRDKVKNHATYYAQKFQGRVTMTRDTSTSTVYMELSSLRSEDTAVY |
| | YCRLGVFDYWGQGTLVTVSS (SEQ ID NO: 1255) |
| 3A7V5-51 | EVQLVQSGAEVKKPGESLKISCKGSGFTFSDAWMGWVRQMPGKGLEWV |
| | AEIRDKVKNHATYYAPSFQGQVTISADKSISTAYLQWSSLKASDTAMYYC |
| | RLGVFDYWGQGTLVTVSS (SEQ ID NO: 1256) |
| 3A7V4-59 | QVQLQESGPGLVKPSETLSLTCTVSGFTFSDAWMGWIRQPPGKGLEWVAE |
| | IRDKVKNHATYYAPSLKSRVTISVDTSKNQFSLKLSSVTAADTAVYYCRL |
| | GVFDYWGQGTLVTVSS (SEQ ID NO: 1257) |
| 3A7V4-39 | QLQLQESGPGLVKPSETLSLTCTVSGFTFSDAWMGWIRQPPGKGLEWVAE |
| | IRDKVKNHATYYAPSLKSRVTISVDTSKNQFSLKLSSVTAADTAVYYCRL |
| | GVFDYWGQGTLVTVSS (SEQ ID NO: 1258) |

Antibody 3B10

| | |
|---|---|
| 3B10V3-15 | EVQLVESGGGLVKPGGSLRLSCAASGLTSNTYTQTWVRQAPGKGLEWES |
| | VIRSKSNNFSTLYAAPVKGRFTISRDD SKNTLYLQMNSLKTEDTAVYYCV |
| | RHKSNRYPGVYWGQGTLVTVSS (SEQ ID NO: 1292) |
| 3B10V3-30 | QVQLVESGGGVVQPGRSLRLSCAASGLTSNTYTQTWVRQAPGKGLEWES |
| | VIRSKSNNFSTLYADSVKGRFTISRDNSKNTLYLQMNSLRAEDTAVYYCV |
| | RHKSNRYPGVYWGQGTLVTVSS (SEQ ID NO: 1293) |
| 3B10V3-23 | EVQLLESGGGLVQPGGSLRLSCAASGLTSNTYTQTWVRQAPGKGLEWES |
| | VIRSKSNNFSTLYADSVKGRFTISRDNSKNTLYLQMNSLRAEDTAVYYCV |
| | RHKSNRYPGVYWGQGTLVTVSS (SEQ ID NO: 1294) |
| 3B10V1-46 | QVQLVQSGAEVKKPGASVKVSCKASGLTSNTYTQTWVRQAPGQGLEWE |
| | SVIRSKSNNFSTLYAQKFQGRVTMTRDTSTSTVYMELSSLRSEDTAVYYC |
| | VRHKSNRYPGVYWGQGTLVTVSS (SEQ ID NO: 1295) |
| 3B10V3-48 | EVQLVESGGGLVQPGGSLRLSCAASGLTSNTYTQTWVRQAPGKGLEWES |
| | VIRSKSNNFSTLYADSVKGRFTISRDNAKNSLYLQMNSLRAEDTAVYYCV |
| | RHKSNRYPGVYWGQGTLVTVSS (SEQ ID NO: 1296) |
| 3B10V1-69 | QVQLVQSGAEVKKPGSSVKVSCKASGLTSNTYTQTWVRQAPGQGLEWES |
| | VIRSKSNNFSTLYAQKFQGRVTITADESTSTAYMELSSLRSEDTAVYYCVR |
| | HKSNRYPGVYWGQGTLVTVSS (SEQ ID NO: 1297) |
| 3B10V3-7 | EVQLVESGGGLVQPGGSLRLSCAASGLTSNTYTQTWVRQAPGKGLEWES |
| | VIRSKSNNFSTLYADSVKGRFTISRDNAKNSLYLQMNSLRAEDTAVYYCV |
| | RHKSNRYPGVYWGQGTLVTVSS (SEQ ID NO: 1296) |
| 3B10V5-51 | EVQLVQSGAEVKKPGESLKISCKGSGLTSNTYTQTWVRQMPGKGLEWES |
| | VIRSKSNNFSTLYAPSFQGQVTISADKSISTAYLQWSSLKASDTAMYYCVR |
| | HKSNRYPGVYWGQGTLVTVSS (SEQ ID NO: 1298) |
| 3B10V4-59 | QVQLQESGPGLVKPSETLSLTCTVSGLTSNTYTQTWIRQPPGKGLEWESVI |
| | RSKSNNFSTLYAPSLKSRVTISVDTSKNQFSLKLSSVTAADTAVYYCVRHK |
| | SNRYPGVYWGQGTLVTVSS (SEQ ID NO: 1299) |
| 3B10V4-39 | QLQLQESGPGLVKPSETLSLTCTVSGLTSNTYTQTWIRQPPGKGLEWESVI |
| | RSKSNNFSTLYAPSLKSRVTISVDTSKNQFSLKLSSVTAADTAVYYCVRHK |
| | SNRYPGVYWGQGTLVTVSS (SEQ ID NO: 1300) |

Antibody 4F11

| | |
|---|---|
| 4F11V5-51 | EVQLVQSGAEVKKPGESLKISCKGSGYPFSNFWITWVRQMPGKGLEWIGD |
| | IYPGSDNSNYNPSFQGQVTISADKSISTAYLQWSSLKASDTAMYYCAREAY |
| | YTNPGFAYWGQGTLVTVSS (SEQ ID NO: 1282) |
| 4F11V1-69 | QVQLVQSGAEVKKPGSSVKVSCKASGYPFSNFWITWVRQAPGQGLEWIG |
| | DIYPGSDNSNYNQKFQGRVTITADESTSTAYMELSSLRSEDTAVYYCAREA |
| | YYTNPGFAYWGQGTLVTVSS (SEQ ID NO: 1283) |
| 4F 11V1-46 | QVQLVQSGAEVKKPGASVKVSCKASGYPFSNFWITWVRQAPGQGLEWIG |
| | DIYPGSDNSNYNQKFQGRVTMTRDTSTSTVYMELSSLRSEDTAVYYCARE |
| | AYYTNPGFAYWGQGTLVTVSS (SEQ ID NO: 1284) |
| 4F11V3-48 | EVQLVESGGGLVQPGGSLRLSCAASGYPFSNFWITWVRQAPGKGLEWIGD |
| | IYPGSDNSNYNDSVKGRFTISRDNAKNSLYLQMNSLRAEDTAVYYCAREA |
| | YYTNPGFAYWGQGTLVTVSS (SEQ ID NO: 1285) |
| 4F11V3-30 | QVQLVESGGGVVQPGRSLRLSCAASGYPFSNFWITWVRQAPGKGLEWIG |
| | DIYPGSDNSNYNDSVKGRFTISRDNSKNTLYLQMNSLRAEDTAVYYCARE |
| | AYYTNPGFAYWGQGTLVTVSS (SEQ ID NO: 1286) |
| 4F11V3-7 | EVQLVESGGGLVQPGGSLRLSCAASGYPFSNFWITWVRQAPGKGLEWIGD |
| | IYPGSDNSNYNDSVKGRFTISRDNAKNSLYLQMNSLRAEDTAVYYCAREA |
| | YYTNPGFAYWGQGTLVTVSS (SEQ ID NO: 1285) |
| 4F11V3-23 | EVQLLESGGGLVQPGGSLRLSCAASGYPFSNFWITWVRQAPGKGLEWIGD |
| | IYPGSDNSNYNDSVKGRFTISRDNSKNTLYLQMNSLRAEDTAVYYCAREA |
| | YYTNPGFAYWGQGTLVTVSS (SEQ ID NO: 1287) |
| 4F11V4-59 | QVQLQESGPGLVKPSETLSLTCTVSGYPFSNFWITWIRQPPGKGLEWIGDIY |
| | PGSDNSNYNPSLKSRVTISVDTSKNQFSLKLSSVTAADTAVYYCAREAYY |
| | TNPGFAYWGQGTLVTVSS (SEQ ID NO: 1288) |
| 4F11V3-15 | EVQLVESGGGLVKPGGSLRLSCAASGYPFSNFWITWVRQAPGKGLEWIGD |
| | IYPGSDNSNYNAPVKGRFTISRDDSKNTLYLQMNSLKTEDTAVYYCAREA |
| | YYTNPGFAYWGQGTLVTVSS (SEQ ID NO: 1289) |
| 4F11V4-39 | QLQLQESGPGLVKPSETLSLTCTVSGYPFSNFWITWIRQPPGKGLEWIGDIY |
| | PGSDNSNYNPSLKSRVTISVDTSKNQFSLKLSSVTAADTAVYYCAREAYY |
| | TNPGFAYWGQGTLVTVSS (SEQ ID NO: 1290) |

Antibody 6H6

| | |
|---|---|
| 6H6V3-15 | EVQLVESGGGLVKPGGSLRLSCAASGFTFSDAWMDWVRQAPGKGLEWW |
| | VAEIRNKVNNHATYYAPVKGRFTISRDDSKNTLYLQMNSLKTEDTAVYY |
| | CCTSLYDGYYLRFAWGQGTLVTVSS (SEQ ID NO: 1302) |
| 6H6V3-7 | EVQLVESGGGLVQPGGSLRLSCAASGFTFSDAWMDWVRQAPGKGLEWW |
| | VAEIRNKVNNHATYYDSVKGRFTISRDNAKNSLYLQMNSLRAEDTAVYY |
| | CCTSLYDGYYLRFAWGQGTLVTVSS (SEQ ID NO: 1303) |
| 6H6V3-23 | EVQLLESGGGLVQPGGSLRLSCAASGFTFSDAWMDWVRQAPGKGLEWW |
| | VAEIRNKVNNHATYYDSVKGRFTISRDNSKNTLYLQMNSLRAEDTAVYY |
| | CCTSLYDGYYLRFAWGQGTLVTVSS (SEQ ID NO: 1304) |
| 6H6V3-48 | EVQLVESGGGLVQPGGSLRLSCAASGFTFSDAWMDWVRQAPGKGLEWW |
| | VAEIRNKVNNHATYYDSVKGRFTISRDNAKNSLYLQMNSLRAEDTAVYY |
| | CCTSLYDGYYLRFAWGQGTLVTVSS (SEQ ID NO: 1303) |
| 6H6V3-30 | QVQLVESGGGVVQPGRSLRLSCAASGFTFSDAWMDWVRQAPGKGLEWW |
| | VAEIRNKVNNHATYYDSVKGRFTISRDNSKNTLYLQMNSLRAEDTAVYY |
| | CCTSLYDGYYLRFAWGQGTLVTVSS (SEQ ID NO: 1305) |
| 6H6V1-46 | QVQLVQSGAEVKKPGASVKVSCKASGFTFSDAWMDWVRQAPGQGLEW |
| | WVAEIRNKVNNHATYYQKFQGRVTMTRDTSTSTVYMELSSLRSEDTAVY |
| | YCCTSLYDGYYLRFAWGQGTLVTVSS (SEQ ID NO: 1306) |
| 6H6V 1-69 | QVQLVQSGAEVKKPGSSVKVSCKASGFTFSDAWMDWVRQAPGQGLEW |
| | WVAEIRNKVNNHATYYQKFQGRVTITADESTSTAYMELSSLRSEDTAVYY |
| | CCTSLYDGYYLRFAWGQGTLVTVSS (SEQ ID NO: 1307) |
| 6H6V5-51 | EVQLVQSGAEVKKPGESLKISCKGSGFTFSDAWMDWVRQMPGKGLEWW |
| | VAEIRNKVNNHATYYPSFQGQVTISADKSISTAYLQWSSLKASDTAMYYC |
| | CTSLYDGYYLRFAWGQGTLVTVSS (SEQ ID NO: 1308) |
| 6H6V4-59 | QVQLQESGPGLVKPSETLSLTCTVSGFTFSDAWMDWIRQPPGKGLEWWV |
| | AEIRNKVNNHATYYPSLKSRVTISVDTSKNQFSLKLSSVTAADTAVYYCCT |
| | SLYDGYYLRFAWGQGTLVTVSS (SEQ ID NO: 1309) |
| 6H6V4-39 | QLQLQESGPGLVKPSETLSLTCTVSGFTFSDAWMDWIRQPPGKGLEWWV |
| | AEIRNKVNNHATYYPSLKSRVTISVDTSKNQFSLKLSSVTAADTAVYYCCT |
| | SLYDGYYLRFAWGQGTLVTVSS (SEQ ID NO: 1310) |

Antibody7A9

| | |
|---|---|
| 7A9V3-15 | EVQLVESGGGLVKPGGSLRLSCAASGFTFNTYSMNWVRQAPGKGLEWVA |
| | HIKTKZNNFATFYAAPVKGRFTISRDDSKNTLYLQMNSLKTEDTAVYYCV |
| | ZHZSNNYPFAYWGQGTLVTVSS (SEQ ID NO: 1312) |
| 7A9V3-48 | EVQLVESGGGLVQPGGSLRLSCAASGFTFNTYSMNWVRQAPGKGLEWVA |
| | HIKTKZNNFATFYADSVKGRFTISRDNAKNSLYLQMNSLRAEDTAVYYCV |
| | ZHZSNNYPFAYWGQGTLVTVSS (SEQ ID NO: 1313) |
| 7A9V3-23 | EVQLLESGGGLVQPGGSLRLSCAASGFTFNTYSMNWVRQAPGKGLEWVA |
| | HIKTKZNNFATFYADSVKGRFTISRDNSKNTLYLQMNSLRAEDTAVYYCV |
| | ZHZSNNYPFAYWGQGTLVTVSS (SEQ ID NO: 1314) |
| 7A9V3-7 | EVQLVESGGGLVQPGGSLRLSCAASGFTFNTYSMNWVRQAPGKGLEWVA |
| | HIKTKZNNFATFYADSVKGRFTISRDNAKNSLYLQMNSLRAEDTAVYYCV |
| | ZHZSNNYPFAYWGQGTLVTVSS (SEQ ID NO: 1313) |
| 7A9V3-30 | QVQLVESGGGVVQPGRSLRLSCAASGFTFNTYSMNWVRQAPGKGLEWV |
| | AHIKTKZNNFATFYADSVKGRFTISRDNSKNTLYLQMNSLRAEDTAVYYC |
| | VZHZSNNYPFAYWGQGTLVTVSS (SEQ ID NO: 1315) |
| 7A9V1-46 | QVQLVQSGAEVKKPGASVKVSCKASGFTFNTYSMNWVRQAPGQGLEWV |
| | AHIKTKZNNFATFYAQKFQGRVTMTRDTSTSTVYMELSSLRSEDTAVYYC |
| | VZHZSNNYPFAYWGQGTLVTVSS (SEQ ID NO: 1316) |
| 7A9V1-69 | QVQLVQSGAEVKKPGSSVKVSCKASGFTFNTYSMNWVRQAPGQGLEWV |
| | AHIKTKZNNFATFYAQKFQGRVTITADESTSTAYMELSSLRSEDTAVYYCV |
| | ZHZSNNYPFAYWGQGTLVTVSS (SEQ ID NO: 1317) |
| 7A9V5-51 | EVQLVQSGAEVKKPGESLKISCKGSGFTFNTYSMNWVRQMPGKGLEWVA |
| | HIKTKZNNFATFYAPSFQGQVTISADKSISTAYLQWSSLKASDTAMYYCVZ |
| | HZSNNYPFAYWGQGTLVTVSS (SEQ ID NO: 1318) |
| 7A9V4-59 | QVQLQESGPGLVKPSETLSLTCTVSGFTFNTYSMNWIRQPPGKGLEWVAHI |
| | KTKZNNFATFYAPSLKSRVTISVDTSKNQFSLKLSSVTAADTAVYYCVZHZ |
| | SNNYPFAYWGQGTLVTVSS (SEQ ID NO: 1319) |
| 7A9V4-30-4 | QVQLQESGPGLVKPSQTLSLTCTVSGFTFNTYSMNWIRQPPGKGLEWVAH |
| | IKTKZNNFATFYAPSLKSRVTISVDTSKNQFSLKLSSVTAADTAVYYCVZH |
| | ZSNNYPFAYWGQGTLVTVSS (SEQ ID NO: 1320) |

Antibody 7B3

| | |
|---|---|
| 7B3V1-46 | QVQLVQSGAEVKKPGASVKVSCKASGYTFTTYWIHWVRQAPGQGLEWIG |
| | RNDPNSGGSNYNQKFQGRVTMTRDTSTSTVYMELSSLRSEDTAVYYCVR |
| | TNWDGDFWGQGTLVTVSS (SEQ ID NO: 1322) |
| 7B3V5-51 | EVQLVQSGAEVKKPGESLKISCKGSGYTFTTYWIHWVRQMPGKGLEWIG |
| | RNDPNSGGSNYNPSFQGQVTISADKSISTAYLQWSSLKASDTAMYYCVRT |
| | NWDGDFWGQGTLVTVSS (SEQ ID NO: 1323) |
| 7B3V1-69 | QVQLVQSGAEVKKPGSSVKVSCKASGYTFTTYWIHWVRQAPGQGLEWIG |
| | RNDPNSGGSNYNQKFQGRVTITADESTSTAYMELSSLRSEDTAVYYCVRT |
| | NWDGDFWGQGTLVTVSS (SEQ ID NO: 1324) |
| 7B3V3-23 | EVQLLESGGGLVQPGGSLRLSCAASGYTFTTYWIHWVRQAPGKGLEWIG |
| | RNDPNSGGSNYNDSVKGRFTISRDNSKNTLYLQMNSLRAEDTAVYYCVR |
| | TNWDGDFWGQGTLVTVSS (SEQ ID NO: 1325) |
| 7B3V3-7 | EVQLVESGGGLVQPGGSLRLSCAASGYTFTTYWIHWVRQAPGKGLEWIG |
| | RNDPNSGGSNYNDSVKGRFTISRDNAKNSLYLQMNSLRAEDTAVYYCVR |
| | TNWDGDFWGQGTLVTVSS (SEQ ID NO: 1326) |
| 7B3V3 -30 | QVQLVESGGGVVQPGRSLRLSCAASGYTFTTYWIHWVRQAPGKGLEWIG |
| | RNDPNSGGSNYNDSVKGRFTISRDNSKNTLYLQMNSLRAEDTAVYYCVR |
| | TNWDGDFWGQGTLVTVSS (SEQ ID NO: 1327) |
| 7B3V3-48 | EVQLVESGGGLVQPGGSLRLSCAASGYTFTTYWIHWVRQAPGKGLEWIG |
| | RNDPNSGGSNYNDSVKGRFTISRDNAKNSLYLQMNSLRAEDTAVYYCVR |
| | TNWDGDFWGQGTLVTVSS (SEQ ID NO: 1326) |
| 7B3V4-59 | QVQLQESGPGLVKPSETLSLTCTVSGYTFTTYWIHWIRQPPGKGLEWIGRN |
| | DPNSGGSNYNPSLKSRVTISVDTSKNQFSLKLSSVTAADTAVYYCVRTNW |
| | DGDFWGQGTLVTVSS (SEQ ID NO: 1328) |
| 7B3V3-15 | EVQLVESGGGLVKPGGSLRLSCAASGYTFTTYWIHWVRQAPGKGLE |
| | WIGRNDPNSGGSNYNAPVKGRFTISRDDSKNTLYLQMNSLKTEDTAV |
| | YYCVRTNWDGDFWGQGTLVTVSS (SEQ ID NO: 1329) |
| 7B3V4-30-4 | QVQLQESGPGLVKPSQTLSLTCTVSGYTFTTYWIHWIRQPPGKGLEWI |
| | GRNDPNSGGSNYNPSLKSRVTISVDTSKNQFSLKLSSVTAADTAVYYC |
| | VRTNWDGDFWGQGTLVTVSS (SEQ ID NO: 1330) |

Antibody 8A1

| | |
|---|---|
| 8A1V5-51 | EVQLVQSGAEVKKPGESLKISCKGSGYAFSNYWMSWVRQMPGKGLE |
| | WIGQIYPGDGDTKYNPSFQGQVTISADKSISTAYLQWSSLKASDTAM |
| | YYCSREKGADYYGSTYSAWFSYWGQGTLVTVSS (SEQ ID NO: 1332) |
| 8A1V1-46 | QVQLVQSGAEVKKPGASVKVSCKASGYAFSNYWMSWVRQAPGQG |
| | LEWIGQIYPGDGDTKYNQKFQGRVTMTRDTSTSTVYMELSSLRSEDT |
| | AVYYCSREKGADYYGSTYSAWFSYWGQGTLVTVSS (SEQ ID NO: 1333) |
| 8A1V3-23 | EVQLLESGGGLVQPGGSLRLSCAASGYAFSNYWMSWVRQAPGKGLE |
| | WIGQIYPGDGDTKYNDSVKGRFTISRDNSKNTLYLQMNSLRAEDTAV |
| | YYCSREKGADYYGSTYSAWFSYWGQGTLVTVSS (SEQ ID NO: 1334) |
| 8A1V1-69 | QVQLVQSGAEVKKPGSSVKVSCKASGYAFSNYWMSWVRQAPGQGL |
| | EWIGQIYPGDGDTKYNQKFQGRVTITADESTSTAYMELSSLRSEDTAV |
| | YYCSREKGADYYGSTYSAWFSYWGQGTLVTVSS (SEQ ID NO: 1335) |
| 8A1V3-7 | EVQLVESGGGLVQPGGSLRLSCAASGYAFSNYWMSWVRQAPGKGLE |
| | WIGQIYPGDGDTKYNDSVKGRFTISRDNAKNSLYLQMNSLRAEDTAV |
| | YYCSREKGADYYGSTYSAWFSYWGQGTLVTVSS (SEQ ID NO: 1336) |
| 8A1V3-48 | EVQLVESGGGLVQPGGSLRLSCAASGYAFSNYWMSWVRQAPGKGLE |
| | WIGQIYPGDGDTKYNDSVKGRFTISRDNAKNSLYLQMNSLRAEDTAV |
| | YYCSREKGADYYGSTYSAWFSYWGQGTLVTVSS (SEQ ID NO: 1336) |
| 8A1V3-30 | QVQLVESGGGVVQPGRSLRLSCAASGYAFSNYWMSWVRQAPGKGL |
| | EWIGQIYPGDGDTKYNDSVKGRFTISRDNSKNTLYLQMNSLRAEDTA |
| | VYYCSREKGADYYGSTYSAWFSYWGQGTLVTVSS (SEQ ID NO: 1337) |
| 8A1V4-59 | QVQLQESGPGLVKPSETLSLTCTVSGYAFSNYWMSWIRQPPGKGLE |
| | WIGQIYPGDGDTKYNPSLKSRVTISVDTSKNQFSLKLSSVTAADTAVY |
| | YCSREKGADYYGSTYSAWFSYWGQGTLVTVSS (SEQ ID NO: 1338) |
| 8A1V3-15 | EVQLVESGGGLVKPGGSLRLSCAASGYAFSNYWMSWVRQAPGKGLE |
| | WIGQIYPGDGDTKYNAPVKGRFTISRDDSKNTLYLQMNSLKTEDTAV |
| | YYCSREKGADYYGSTYSAWFSYWGQGTLVTVSS (SEQ ID NO: 1339) |
| 8A1V4-30-4 | QVQLQESGPGLVKPSQTLSLTCTVSGYAFSNYWMSWIRQPPGKGLE |
| | WIGQIYPGDGDTKYNPSLKSRVTISVDTSKNQFSLKLSSVTAADTAVY |
| | YCSREKGADYYGSTYSAWFSYWGQGTLVTVSS (SEQ ID NO: 1340) |

Antibody 9F5

| | |
|---|---|
| 9F5V5-51 | EVQLVQSGAEVKKPGESLKISCKGSGYAFSSSWMNWVRQMPGKGLE |
| | WIGRIYPGDGDTNYNPSFQGQVTISADKSISTAYLQWSSLKASDTAM |
| | YYCARLLRNQPGESYAMDYWGQGTLVTVSS (SEQ ID NO: 1342) |
| 9F5V1-46 | QVQLVQSGAEVKKPGASVKVSCKASGYAFSSSWMNWVRQAPGQGL |
| | EWIGRIYPGDGDTNYNQKFQGRVTMTRDTSTSTVYMELSSLRSEDTA |
| | VYYCARLLRNQPGESYAMDYWGQGTLVTVSS (SEQ ID NO: 1343) |
| 9F5V1-69 | QVQLVQSGAEVKKPGSSVKVSCKASGYAFSSSWMNWVRQAPGQGL |
| | EWIGRIYPGDGDTNYNQKFQGRVTITADESTSTAYMELSSLRSEDTAV |
| | YYCARLLRNQPGESYAMDYWGQGTLVTVSS (SEQ ID NO: 1344) |
| 9F5V3 -23 | EVQLLESGGGLVQPGGSLRLSCAASGYAFSSSWMNWVRQAPGKGLE |
| | WIGRIYPGDGDTNYNDSVKGRFTISRDNSKNTLYLQMNSLRAEDTAV |
| | YYCARLLRNQPGESYAMDYWGQGTLVTVSS (SEQ ID NO: 1345) |
| 9F5V3-7 | EVQLVESGGGLVQPGGSLRLSCAASGYAFSSSWMNWVRQAPGKGLE |
| | WIGRIYPGDGDTNYNDSVKGRFTISRDNAKNSLYLQMNSLRAEDTAV |
| | YYCARLLRNQPGESYAMDYWGQGTLVTVSS (SEQ ID NO: 1346) |
| 9F5V3-48 | EVQLVESGGGLVQPGGSLRLSCAASGYAFSSSWMNWVRQAPGKGLE |
| | WIGRIYPGDGDTNYNDSVKGRFTISRDNAKNSLYLQMNSLRAEDTAV |
| | YYCARLLRNQPGESYAMDYWGQGTLVTVSS (SEQ ID NO: 1346) |
| 9F5V3-30 | QVQLVESGGGVVQPGRSLRLSCAASGYAFSSSWMNWVRQAPGKGLE |
| | WIGRIYPGDGDTNYNDSVKGRFTISRDNSKNTLYLQMNSLRAEDTAV |
| | YYCARLLRNQPGESYAMDYWGQGTLVTVSS (SEQ ID NO: 1347) |
| 9F5V4-59 | QVQLQESGPGLVKPSETLSLTCTVSGYAFSSSWMNWIRQPPGKGLE |
| | WIGRIYPGDGDTNYNPSLKSRVTISVDTSKNQFSLKLSSVTAADTAVY |
| | YCARLLRNQPGESYAMDYWGQGTLVTVSS (SEQ ID NO: 1348) |
| 9F5V3-15 | EVQLVESGGGLVKPGGSLRLSCAASGYAFSSSWMNWVRQAPGKGLE |
| | WIGRIYPGDGDTNYNAPVKGRFTISRDDSKNTLYLQMNSLKTEDTAV |
| | YYCARLLRNQPGESYAMDYWGQGTLVTVSS (SEQ ID NO: 1349) |
| 9F5V4-30-4 | QVQLQESGPGLVKPSQTLSLTCTVSGYAFSSSWMNWIRQPPGKGLE |
| | WIGRIYPGDGDTNYNPSLKSRVTISVDTSKNQFSLKLSSVTAADTAVY |
| | YCARLLRNQPGESYAMDYWGQGTLVTVSS (SEQ ID NO: 1350) |
| 9F5-H1 | QVQLVQSGAEVKKPGASVKVSCKASGYAFSSSWMNWVRQAPGQGL |
| | EWMGRIYPGDGDTNYAQKFQGRVTMTRDTSTSTVYMELSSLRSEDT |
| | AVYYCARLLRNQPGESYAMDYWGQGTLVTVSS (SEQ ID NO: 1665) |
| 9F5-H2 | QVQLVQSGAEVKKPGASVKVSCKASGYAFSSSWMNWVRQAPGQGL |
| | EWIGRIYPGDGDTNYAQKFQGRVTMTADTSTSTVYMELSSLRSEDTA |
| | VYYCARLLRNQPGESYAMDYWGQGTLVTVSS (SEQ ID NO: 1666) |
| 9F5-H3 | QVQLVQSGAEVKKPGASLKISCKASGYAFSSSWMNWVRQAPGQGLE |
| | WIGRIYPGDGDTNYAQKFQGRATLTADTSTSTAYMELSSLRSEDTAV |
| | YYCARLLRNQPGESYAMDYWGQGALVTVSS (SEQ ID NO: 1667) |

Antibody 9G1

| | |
|---|---|
| 9G1V5-51 | EVQLVQSGAEVKKPGESLKISCKGSGYIFTTYWIHWVRQMPGKGLE |
| | WIGRIDPNNGDTNYNPSFQGQVTISADKSISTAYLQWSSLKASDTAM |
| | YYCVMTGTDFDYWGQGTLVTVSS (SEQ ID NO: 1352) |
| 9G1V1-46 | QVQLVQSGAEVKKPGASVKVSCKASGYIFTTYWIHWVRQAPGQGLE |
| | WIGRIDPNNGDTNYNQKFQGRVTMTRDTSTSTVYMELSSLRSEDTA |
| | VYYCVMTGTDFDYWGQGTLVTVSS (SEQ ID NO: 1353) |
| 9G1V1-69 | QVQLVQSGAEVKKPGSSVKVSCKASGYIFTTYWIHWVRQAPGQGLE |
| | WIGRIDPNNGDTNYNQKFQGRVTITADESTSTAYMELSSLRSEDTAV |
| | YYCVMTGTDFDYWGQGTLVTVSS (SEQ ID NO: 1354) |
| 9G1V3-23 | EVQLLESGGGLVQPGGSLRLSCAASGYIFTTYWIHWVRQAPGKGLEW |
| | IGRIDPNNGDTNYNDSVKGRFTISRDNSKNTLYLQMNSLRAEDTAVY |
| | YCVMTGTDFDYWGQGTLVTVSS (SEQ ID NO: 1355) |
| 9G1V3-30 | QVQLVESGGGVVQPGRSLRLSCAASGYIFTTYWIHWVRQAPGKGLE |
| | WIGRIDPNNGDTNYNDSVKGRFTISRDNSKNTLYLQMNSLRAEDTA |
| | VYYCVMTGTDFDYWGQGTLVTVSS (SEQ ID NO: 1356) |
| 9G1V3-7 | EVQLVESGGGLVQPGGSLRLSCAASGYIFTTYWIHWVRQAPGKGLE |
| | WIGRIDPNNGDTNYNDSVKGRFTISRDNAKNSLYLQMNSLRAEDTAV |
| | YYCVMTGTDFDYWGQGTLVTVSS (SEQ ID NO: 1357) |
| 9G1V3-48 | EVQLVESGGGLVQPGGSLRLSCAASGYIFTTYWIHWVRQAPGKGLE |
| | WIGRIDPNNGDTNYNDSVKGRFTISRDNAKNSLYLQMNSLRAEDTAV |
| | YYCVMTGTDFDYWGQGTLVTVSS (SEQ ID NO: 1357) |
| 9G1V4-59 | QVQLQESGPGLVKPSETLSLTCTVSGYIFTTYWIHWIRQPPGKGLEWI |
| | GRIDPNNGDTNYNPSLKSRVTISVDTSKNQFSLKLSSVTAADTAVYY |
| | CVMTGTDFDYWGQGTLVTVSS (SEQ ID NO: 1358) |
| 9G1V3-15 | EVQLVESGGGLVKPGGSLRLSCAASGYIFTTYWIHWVRQAPGKGLE |
| | WIGRIDPNNGDTNYNAPVKGRFTISRDDSKNTLYLQMNSLKTEDTAV |
| | YYCVMTGTDFDYWGQGTLVTVSS (SEQ ID NO: 1359) |
| 9G1V4-30-4 | QVQLQESGPGLVKPSQTLSLTCTVSGYIFTTYWIHWIRQPPGKGLEWI |
| | GRIDPNNGDTNYNPSLKSRVTISVDTSKNQFSLKLSSVTAADTAVYY |
| | CVMTGTDFDYWGQGTLVTVSS (SEQ ID NO: 1360) |

Antibody 9G3

| | |
|---|---|
| 9G3V3 -15 | EVQLVESGGGLVKPGGSLRLSCAASGFNFNTYAMKWVRQAPGKGLE |
| | WIARIRSNSNDYATNYSAPVKGRFTISRDDSKNTLYLQMNSLKTEDTA |
| | VYYCVGHKINNYPFAHWGQGTLVTVSS (SEQ ID NO: 1456) |
| 9G3V3-23 | EVQLLESGGGLVQPGGSLRLSCAASGFNFNTYAMKWVRQAPGKGLE |
| | WIARIRSNSNDYATNYSDSVKGRFTISRDNSKNTLYLQMNSLRAEDT |
| | AVYYCVGHKINNYPFAHWGQGTLVTVSS (SEQ ID NO: 1457) |
| 9G3V3-30 | QVQLVESGGGVVQPGRSLRLSCAASGFNFNTYAMKWVRQAPGKGLE |
| | WIARIRSNSNDYATNYSDSVKGRFTISRDNSKNTLYLQMNSLRAEDTAV |
| | YYCVGHKINNYPFAHWGQGTLVTVSS (SEQ ID NO: 1458) |
| 9G3V3-48 | EVQLVESGGGLVQPGGSLRLSCAASGFNFNTYAMKWVRQAPGKGLE |
| | WIARIRSNSNDYATNYSDSVKGRFTISRDNAKNSLYLQMNSLRAEDT |
| | AVYYCVGHKINNYPFAHWGQGTLVTVSS (SEQ ID NO: 1459) |
| 9G3V3-7 | EVQLVESGGGLVQPGGSLRLSCAASGFNFNTYAMKWVRQAPGKGLE |
| | WIARIRSNSNDYATNYSDSVKGRFTISRDNAKNSLYLQMNSLRAEDT |
| | AVYYCVGHKINNYPFAHWGQGTLVTVSS (SEQ ID NO: 1460) |
| 9G3V1-69 | QVQLVQSGAEVKKPGSSVKVSCKASGFNFNTYAMKWVRQAPGQGL |
| | EWIARIRSNSNDYATNYSQKFQGRVTITADESTSTAYMELSSLRSEDT |
| | AVYYCVGHKINNYPFAHWGQGTLVTVSS (SEQ ID NO: 1461) |
| 9G3V1-46 | QVQLVQSGAEVKKPGASVKVSCKASGFNFNTYAMKWVRQAPGQGL |
| | EWIARIRSNSNDYATNYSQKFQGRVTMTRDTSTSTVYMELSSLRSEDT |
| | AVYYCVGHKINNYPFAHWGQGTLVTVSS (SEQ ID NO: 1462) |
| 9G3V5-51 | EVQLVQSGAEVKKPGESLKISCKGSGFNFNTYAMKWVRQMPGKGL |
| | EWIARIRSNSNDYATNYSPSFQGQVTISADKSISTAYLQWSSLKASDT |
| | AMYYCVGHKINNYPFAHWGQGTLVTVSS (SEQ ID NO: 1463) |
| 9G3V4-59 | QVQLQESGPGLVKPSETLSLTCTVSGFNFNTYAMKWIRQPPGKGLEWI |
| | ARIRSNSNDYATNYSPSLKSRVTISVDTSKNQFSLKLSSVTAADTAVYY |
| | CVGHKINNYPFAHWGQGTLVTVSS (SEQ ID NO: 1464) |
| 9G3V4-30-4 | QVQLQESGPGLVKPSQTLSLTCTVSGFNFNTYAMKWIRQPPGKGLEWI |
| | ARIRSNSNDYATNYSPSLKSRVTISVDTSKNQFSLKLSSVTAADTAVYY |
| | CVGHKINNYPFAHWGQGTLVTVSS (SEQ ID NO: 1465) |

Antibody10A9

| | |
|---|---|
| 10A9V5-51 | EVQLVQSGAEVKKPGESLKISCKGSGYPFSNFWITWVRQMPGKGLE |
| | WIGDIYPGSDNRNFNPSFQGQVTISADKSISTAYLQWSSLKASDTAMY |
| | YCAREAYYTNPGFAYWGQGTLVTVSS (SEQ ID NO: 1362) |
| 10A9V1-69 | QVQLVQSGAEVKKPGSSVKVSCKASGYPFSNFWITWVRQAPGQGLE |
| | WIGDIYPGSDNRNFNQKFQGRVTITADESTSTAYMELSSLRSEDTAVY |
| | YCAREAYYTNPGFAYWGQGTLVTVSS (SEQ ID NO: 1363) |
| 10A9V1-46 | QVQLVQSGAEVKKPGASVKVSCKASGYPFSNFWITWVRQAPGQGLE |
| | WIGDIYPGSDNRNFNQKFQGRVTMTRDTSTSTVYMELSSLRSEDTAV |
| | YYCAREAYYTNPGFAYWGQGTLVTVSS (SEQ ID NO: 1364) |
| 10A9V3-48 | EVQLVESGGGLVQPGGSLRLSCAASGYPFSNFWITWVRQAPGKGLE |
| | WIGDIYPGSDNRNFNDSVKGRFTISRDNAKNSLYLQMNSLRAEDTAV |
| | YYCAREAYYTNPGFAYWGQGTLVTVSS (SEQ ID NO: 1365) |
| 10A9V3-7 | EVQLVESGGGLVQPGGSLRLSCAASGYPFSNFWITWVRQAPGKGLE |
| | WIGDIYPGSDNRNFNDSVKGRFTISRDNAKNSLYLQMNSLRAEDTAV |
| | YYCAREAYYTNPGFAYWGQGTLVTVSS (SEQ ID NO: 1365) |
| 10A9V3-30 | QVQLVESGGGVVQPGRSLRLSCAASGYPFSNFWITWVRQAPGKGLE |
| | WIGDIYPGSDNRNFNDSVKGRFTISRDNSKNTLYLQMNSLRAEDTAV |
| | YYCAREAYYTNPGFAYWGQGTLVTVSS (SEQ ID NO: 1366) |
| 10A9V3-23 | EVQLLESGGGLVQPGGSLRLSCAASGYPFSNFWITWVRQAPGKGLE |
| | WIGDIYPGSDNRNFNDSVKGRFTISRDNSKNTLYLQMNSLRAEDTAV |
| | YYCAREAYYTNPGFAYWGQGTLVTVSS (SEQ ID NO: 1367) |
| 10A9V4-59 | QVQLQESGPGLVKPSETLSLTCTVSGYPFSNFWITWIRQPPGKGLEWI |
| | GDIYPGSDNRNFNPSLKSRVTISVDTSKNQFSLKLSSVTAADTAVYYC |
| | AREAYYTNPGFAYWGQGTLVTVSS (SEQ ID NO: 1368) |
| 10A9V3-15 | EVQLVESGGGLVKPGGSLRLSCAASGYPFSNFWITWVRQAPGKGLE |
| | WIGDIYPGSDNRNFNAPVKGRFTISRDDSKNTLYLQMNSLKTEDTAV |
| | YYCAREAYYTNPGFAYWGQGTLVTVSS (SEQ ID NO: 1369) |
| 10A9V4-39 | QLQLQESGPGLVKPSETLSLTCTVSGYPFSNFWITWIRQPPGKGLEWI |
| | GDIYPGSDNRNFNPSLKSRVTISVDTSKNQFSLKLSSVTAADTAVYYC |
| | AREAYYTNPGFAYWGQGTLVTVSS (SEQ ID NO: 1370) |

Antibody11A8

| | |
|---|---|
| 11A8V3-15 | EVQLVESGGGLVKPGGSLRLSCAASGFNFNTYAMNWVRQAPGKGLE |
| | WVARIRSKSNNYATYYAAPVKGRFTISRDDSKNTLYLQMNSLKTEDT |
| | AVYYCVRHYSNYGWGFAYWGQGTLVTVSS (SEQ ID NO: 1372) |
| 11A8V3-48 | EVQLVESGGGLVQPGGSLRLSCAASGFNFNTYAMNWVRQAPGKGLE |
| | WVARIRSKSNNYATYYADSVKGRFTISRDNAKNSLYLQMNSLRAED |
| | TAVYYCVRHYSNYGWGFAYWGQGTLVTVSS (SEQ ID NO: 1373) |
| 11A8V3-23 | EVQLLESGGGLVQPGGSLRLSCAASGFNFNTYAMNWVRQAPGKGLE |
| | WVARIRSKSNNYATYYADSVKGRFTISRDNSKNTLYLQMNSLRAEDT |
| | AVYYCVRHYSNYGWGFAYWGQGTLVTVSS (SEQ ID NO: 1374) |
| 11A8V3-30 | QVQLVESGGGVVQPGRSLRLSCAASGFNFNTYAMNWVRQAPGKGL |
| | EWVARIRSKSNNYATYYADSVKGRFTISRDNSKNTLYLQMNSLRAED |
| | TAVYYCVRHYSNYGWGFAYWGQGTLVTVSS (SEQ ID NO: 1375) |
| 11A8V3-7 | EVQLVESGGGLVQPGGSLRLSCAASGFNFNTYAMNWVRQAPGKGLE |
| | WVARIRSKSNNYATYYADSVKGRFTISRDNAKNSLYLQMNSLRAED |
| | TAVYYCVRHYSNYGWGFAYWGQGTLVTVSS (SEQ ID NO: 1373) |
| 11A8V1-69 | QVQLVQSGAEVKKPGSSVKVSCKASGFNFNTYAMNWVRQAPGQGL |
| | EWVARIRSKSNNYATYYAQKFQGRVTITADESTSTAYMELSSLRSED |
| | TAVYYCVRHYSNYGWGFAYWGQGTLVTVSS (SEQ ID NO: 1376) |
| 11A8V1-46 | QVQLVQSGAEVKKPGASVKVSCKASGFNFNTYAMNWVRQAPGQGL |
| | EWVARIRSKSNNYATYYAQKFQGRVTMTRDTSTSTVYMELSSLRSE |
| | DTAVYYCVRHYSNYGWGFAYWGQGTLVTVSS (SEQ ID NO: 1377) |
| 11A8V5-51 | EVQLVQSGAEVKKPGESLKISCKGSGFNFNTYAMNWVRQMPGKGLE |
| | WVARIRSKSNNYATYYAPSFQGQVTISADKSISTAYLQWSSLKASDT |
| | AMYYCVRHYSNYGWGFAYWGQGTLVTVSS (SEQ ID NO: 1378) |
| 11A8V4-59 | QVQLQESGPGLVKPSETLSLTCTVSGFNFNTYAMNWIRQPPGKGLEW |
| | VARIRSKSNNYATYYAPSLKSRVTISVDTSKNQFSLKLSSVTAADTAV |
| | YYCVRHYSNYGWGFAYWGQGTLVTVSS (SEQ ID NO: 1379) |
| 11A8V4-39 | QLQLQESGPGLVKPSETLSLTCTVSGFNFNTYAMNWIRQPPGKGLEW |
| | VARIRSKSNNYATYYAPSLKSRVTISVDTSKNQFSLKLSSVTAADTAV |
| | YYCVRHYSNYGWGFAYWGQGTLVTVSS (SEQ ID NO: 1380) |

Antibody12D9

| | |
|---|---|
| 12D9V1-46 | QVQLVQSGAEVKKPGASVKVSCKASGYTFSDYYIHWVRQAPGQGLE |
| | WIGYIYPNNGDNGYNQKFQGRVTMTRDTSTSTVYMELSSLRSEDTA |
| | VYYCARRGYYGGSYDYWGQGTLVTVSS (SEQ ID NO: 1382) |
| 12D9V5-51 | EVQLVQSGAEVKKPGESLKISCKGSGYTFSDYYIHWVRQMPGKGLE |
| | WIGYIYPNNGDNGYNPSFQGQVTISADKSISTAYLQWSSLKASDTAM |
| | YYCARRGYYGGSYDYWGQGTLVTVSS (SEQ ID NO: 1383) |
| 12D9V1-69 | QVQLVQSGAEVKKPGSSVKVSCKASGYTFSDYYIHWVRQAPGQGLE |
| | WIGYIYPNNGDNGYNQKFQGRVTITADESTSTAYMELSSLRSEDTAV |
| | YYCARRGYYGGSYDYWGQGTLVTVSS (SEQ ID NO: 1384) |
| 12D9V3-48 | EVQLVESGGGLVQPGGSLRLSCAASGYTFSDYYIHWVRQAPGKGLE |
| | WIGYIYPNNGDNGYNDSVKGRFTISRDNAKNSLYLQMNSLRAEDTA |
| | VYYCARRGYYGGSYDYWGQGTLVTVSS (SEQ ID NO: 1385) |
| 12D9V3-30 | QVQLVESGGGVVQPGRSLRLSCAASGYTFSDYYIHWVRQAPGKGLE |
| | WIGYIYPNNGDNGYNDSVKGRFTISRDNSKNTLYLQMNSLRAEDTAV |
| | YYCARRGYYGGSYDYWGQGTLVTVSS (SEQ ID NO: 1386) |
| 12D9V3-23 | EVQLLESGGGLVQPGGSLRLSCAASGYTFSDYYIHWVRQAPGKGLE |
| | WIGYIYPNNGDNGYNDSVKGRFTISRDNSKNTLYLQMNSLRAEDTAV |
| | YYCARRGYYGGSYDYWGQGTLVTVSS (SEQ ID NO: 1387) |
| 12D9V3-7 | EVQLVESGGGLVQPGGSLRLSCAASGYTFSDYYIHWVRQAPGKGLE |
| | WIGYIYPNNGDNGYNDSVKGRFTISRDNAKNSLYLQMNSLRAEDTA |
| | VYYCARRGYYGGSYDYWGQGTLVTVSS (SEQ ID NO: 1385) |
| 12D9V4-59 | QVQLQESGPGLVKPSETLSLTCTVSGYTFSDYYIHWIRQPPGKGLEWI |
| | GYIYPNNGDNGYNPSLKSRVTISVDTSKNQFSLKLSSVTAADTAVYY |
| | CARRGYYGGSYDYWGQGTLVTVSS (SEQ ID NO: 1388) |
| 12D9V3-15 | EVQLVESGGGLVKPGGSLRLSCAASGYTFSDYYIHWVRQAPGKGLE |
| | WIGYIYPNNGDNGYNAPVKGRFTISRDDSKNTLYLQMNSLKTEDTAV |
| | YYCARRGYYGGSYDYWGQGTLVTVSS (SEQ ID NO: 1389) |
| 12D9V4-30-4 | QVQLQESGPGLVKPSQTLSLTCTVSGYTFSDYYTHWIRQPPGKGLEWI |
| | GYIYPNNGDNGYNPSLKSRVTISVDTSKNQFSLKLSSVTAADTAVYY |
| | CARRGYYGGSYDYWGQGTLVTVSS (SEQ ID NO: 1390) |

Antibody 12F9

| | |
|---|---|
| 12F9V3-15 | EVQLVESGGGLVKPGGSLRLSCAASGFRFNTYAMTWVRQAPGKGLE |
| | WEGVIRRKSSNFATLYAAPVKGRFTISRDDSKNTLYLQMNSLKTEDT |
| | AVYYCVRHKSNKYPFVYWGQGTLVTVSS (SEQ ID NO: 1392) |
| 12F9V3-23 | EVQLLESGGGLVQPGGSLRLSCAASGFRFNTYAMTWVRQAPGKGLE |
| | WEGVIRRKSSNFATLYADSVKGRFTISRDNSKNTLYLQMNSLRAEDT |
| | AVYYCVRHKSNKYPFVYWGQGTLVTVSS (SEQ ID NO: 1393) |
| 12F9V3-48 | EVQLVESGGGLVQPGGSLRLSCAASGFRFNTYAMTWVRQAPGKGLE |
| | WEGVIRRKSSNFATLYADSVKGRFTISRDNAKNSLYLQMNSLRAEDT |
| | AVYYCVRHKSNKYPFVYWGQGTLVTVSS (SEQ ID NO: 1394) |
| 12F9V3-30 | QVQLVESGGGVVQPGRSLRLSCAASGFRFNTYAMTWVRQAPGKGLE |
| | WEGVIRRKSSNFATLYADSVKGRFTISRDNSKNTLYLQMNSLRAEDT |
| | AVYYCVRHKSNKYPFVYWGQGTLVTVSS (SEQ ID NO: 1395) |
| 12F9V3-7 | EVQLVESGGGLVQPGGSLRLSCAASGFRFNTYAMTWVRQAPGKGLE |
| | WEGVIRRKSSNFATLYADSVKGRFTISRDNAKNSLYLQMNSLRAEDT |
| | AVYYCVRHKSNKYPFVYWGQGTLVTVSS (SEQ ID NO: 1394) |
| 12F9V1-69 | QVQLVQSGAEVKKPGSSVKVSCKASGFRFNTYAMTWVRQAPGQGL |
| | EWEGVIRRKSSNFATLYAQKFQGRVTITADESTSTAYMELSSLRSEDT |
| | AVYYCVRHKSNKYPFVYWGQGTLVTVSS (SEQ ID NO: 1396) |
| 12F9V1-46 | QVQLVQSGAEVKKPGASVKVSCKASGFRFNTYAMTWVRQAPGQGL |
| | EWEGVIRRKSSNFATLYAQKFQGRVTMTRDTSTSTVYMELSSLRSED |
| | TAVYYCVRHKSNKYPFVYWGQGTLVTVSS (SEQ ID NO: 1397) |
| 12F9V5-51 | EVQLVQSGAEVKKPGESLKISCKGSGFRFNTYAMTWVRQMPGKGLE |
| | WEGVIRRKSSNFATLYAPSFQGQVTISADKSISTAYLQWSSLKASDTA |
| | MYYCVRHKSNKYPFVYWGQGTLVTVSS (SEQ ID NO: 1398) |
| 12F9V4-59 | QVQLQESGPGLVKPSETLSLTCTVSGFRFNTYAMTWIRQPPGKGLEW |
| | EGVIRRKSSNFATLYAPSLKSRVTISVDTSKNQFSLKLSSVTAADTAV |
| | YYCVRHKSNKYPFVYWGQGTLVTVSS (SEQ ID NO: 1399) |
| 12F9V4-39 | QLQLQESGPGLVKPSETLSLTCTVSGFRFNTYAMTWIRQPPGKGLEW |
| | EGVIRRKSSNFATLYAPSLKSRVTISVDTSKNQFSLKLSSVTAADTAV |
| | YYCVRHKSNKYPFVYWGQGTLVTVSS (SEQ ID NO: 1400) |

Antibodyl0C1

| | |
|---|---|
| 10C1V3-15 | EVQLVESGGGLVKPGGSLRLSCAASGFTFSDAWMDWVRQAPGKGLE |
| | WVAEIRNKINNHATYYAAPVKGRFTISRDDSKNTLYLQMNSLKTEDT |
| | AVYYCTSLYDGSYLRFAYWGQGTLVTVSS (SEQ ID NO: 1467) |
| 10C1V3-7 | EVQLVESGGGLVQPGGSLRLSCAASGFTFSDAWMDWVRQAPGKGLE |
| | WVAEIRNKINNHATYYADSVKGRFTISRDNAKNSLYLQMNSLRAEDT |
| | AVYYCTSLYDGSYLRFAYWGQGTLVTVSS (SEQ ID NO: 1468) |
| 10C1V3-23 | EVQLLESGGGLVQPGGSLRLSCAASGFTFSDAWMDWVRQAPGKGLE |
| | WVAEIRNKINNHATYYADSVKGRFTISRDNSKNTLYLQMNSLRAEDT |
| | AVYYCTSLYDGSYLRFAYWGQGTLVTVSS (SEQ ID NO: 1469) |
| 10C1V3-30 | QVQLVESGGGVVQPGRSLRLSCAASGFTFSDAWMDWVRQAPGKGL |
| | EWVAEIRNKINNHATYYADSVKGRFTISRDNSKNTLYLQMNSLRAED |
| | TAVYYCTSLYDGSYLRFAYWGQGTLVTVSS (SEQ ID NO: 1470) |
| 10C1V3-48 | EVQLVESGGGLVQPGGSLRLSCAASGFTFSDAWMDWVRQAPGKGLE |
| | WVAEIRNKINNHATYYADSVKGRFTISRDNAKNSLYLQMNSLRAEDT |
| | AVYYCTSLYDGSYLRFAYWGQGTLVTVSS (SEQ ID NO: 1471) |
| 10C1V1-69 | QVQLVQSGAEVKKPGSSVKVSCKASGFTFSDAWMDWVRQAPGQGL |
| | EWVAEIRNKINNHATYYAQKFQGRVTITADESTSTAYMELSSLRSEDT |
| | AVYYCTSLYDGSYLRFAYWGQGTLVTVSS (SEQ ID NO: 1472) |
| 10C1V1-46 | QVQLVQSGAEVKKPGASVKVSCKASGFTFSDAWMDWVRQAPGQGL |
| | EWVAEIRNKINNHATYYAQKFQGRVTMTRDTSTSTVYMELSSLRSED |
| | TAVYYCTSLYDGSYLRFAYWGQGTLVTVSS (SEQ ID NO: 1473) |
| 10C1V5-51 | EVQLVQSGAEVKKPGESLKISCKGSGFTFSDAWMDWVRQMPGKGLE |
| | WVAEIRNKINNHATYYAPSFQGQVTISADKSISTAYLQWSSLKASDTA |
| | MYYCTSLYDGSYLRFAYWGQGTLVTVSS (SEQ ID NO: 1474) |
| 10C1V4-59 | QVQLQESGPGLVKPSETLSLTCTVSGFTFSDAWMDWIRQPPGKGLEW |
| | VAEIRNKINNHATYYAPSLKSRVTISVDTSKNQFSLKLSSVTAADTAV |
| | YYCTSLYDGSYLRFAYWGQGTLVTVSS (SEQ ID NO: 1475) |
| 10C1V4-30-4 | QVQLQESGPGLVKPSQTLSLTCTVSGFTFSDAWMDWIRQPPGKGLEW |
| | VAEIRNKINNHATYYAPSLKSRVTISVDTSKNQFSLKLSSVTAADTAV |
| | YYCTSLYDGSYLRFAYWGQGTLVTVSS (SEQ ID NO: 1476) |

Antibody 7E9

| | |
|---|---|
| 7E9V1-46 | QVQLVQSGAEVKKPGASVKVSCKASGYTFTEYTMIHWVRQAPGQGL |
| | EWIGGINPNNGGTSYKQKFQGRVTMTRDTSTSTVYMELSSLRSEDTA |
| | VYYCARGGSHYYAMDYWGQGTLVTVSS (SEQ ID NO: 1478) |
| 7E9V1-69 | QVQLVQSGAEVKKPGSSVKVSCKASGYTFTEYTMIHWVRQAPGQGL |
| | EWIGGINPNNGGTSYKQKFQGRVTITADESTSTAYMELSSLRSEDTAV |
| | YYCARGGSHYYAMDYWGQGTLVTVSS (SEQ ID NO: 1479) |
| 7E9V5-51 | EVQLVQSGAEVKKPGESLKISCKGSGYTFTEYTMIHWVRQMPGKGLE |
| | WIGGINPNNGGTSYKPSFQGQVTISADKSISTAYLQWSSLKASDTAMY |
| | YCARGGSHYYAMDYWGQGTLVTVSS (SEQ ID NO: 1480) |
| 7E9V3-23 | EVQLLESGGGLVQPGGSLRLSCAASGYTFTEYTMEIWVRQAPGKGLE |
| | WIGGINPNNGGTSYKDSVKGRFTISRDNSKNTLYLQMNSLRAEDTAV |
| | YYCARGGSHYYAMDYWGQGTLVTVSS (SEQ ID NO: 1481) |
| 7E9V3-30 | QVQLVESGGGVVQPGRSLRLSCAASGYTFTEYTMEIWVRQAPGKGLE |
| | WIGGINPNNGGTSYKDSVKGRFTISRDNSKNTLYLQMNSLRAEDTAV |
| | YYCARGGSHYYAMDYWGQGTLVTVSS (SEQ ID NO: 1482) |
| 7E9V3-48 | EVQLVESGGGLVQPGGSLRLSCAASGYTFTEYTMEIWVRQAPGKGLE |
| | WIGGINPNNGGTSYKDSVKGRFTISRDNAKNSLYLQMNSLRAEDTAV |
| | YYCARGGSHYYAMDYWGQGTLVTVSS (SEQ ID NO: 1483) |
| 7E9V3-7 | EVQLVESGGGLVQPGGSLRLSCAASGYTFTEYTMEIWVRQAPGKGLE |
| | WIGGINPNNGGTSYKDSVKGRFTISRDNAKNSLYLQMNSLRAEDTAV |
| | YYCARGGSHYYAMDYWGQGTLVTVSS (SEQ ID NO: 1484) |
| 7E9V4-59 | QVQLQESGPGLVKPSETLSLTCTVSGYTFTEYTMEIWIRQPPGKGLEWI |
| | GGINPNNGGTSYKPSLKSRVTISVDTSKNQFSLKLSSVTAADTAVYYC |
| | ARGGSHYYAMDYWGQGTLVTVSS (SEQ ID NO: 1485) |
| 7E9V3-15 | EVQLVESGGGLVKPGGSLRLSCAASGYTFTEYTMEIWVRQAPGKGLE |
| | WIGGINPNNGGTSYKAPVKGRFTISRDDSKNTLYLQMNSLKTEDTAV |
| | YYCARGGSHYYAMDYWGQGTLVTVSS (SEQ ID NO: 1486) |
| 7E9V4-39 | QLQLQESGPGLVKPSETLSLTCTVSGYTFTEYTMEIWIRQPPGKGLEWI |
| | GGINPNNGGTSYKPSLKSRVTISVDTSKNQFSLKLSSVTAADTAVYYC |
| | ARGGSHYYAMDYWGQGTLVTVSS (SEQ ID NO: 1487) |

Antibody 8C3

| | |
|---|---|
| 8C3V1-46 | QVQLVQSGAEVKKPGASVKVSCKASGYSFTGYYMEIWVRQAPGQGL |
| | EWIGRVNPNNGGTSYNQKFQGRVTMTRDTSTSTVYMELSSLRSEDTA |
| | VYYCVLTGGYFDYWGQGTLVTVSS (SEQ ID NO: 1489) |
| 8C3V5-51 | EVQLVQSGAEVKKPGESLKISCKGSGYSFTGYYMEIWVRQMPGKGLE |
| | WIGRVNPNNGGTSYNPSFQGQVTISADKSISTAYLQWSSLKASDTAM |
| | YYCVLTGGYFDYWGQGTLVTVSS (SEQ ID NO: 1490) |
| 8C3V3-23 | EVQLLESGGGLVQPGGSLRLSCAASGYSFTGYYMEIWVRQAPGKGLE |
| | WIGRVNPNNGGTSYNDSVKGRFTISRDNSKNTLYLQMNSLRAEDTA |
| | VYYCVLTGGYFDYWGQGTLVTVSS (SEQ ID NO: 1491) |
| 8C3V1-69 | QVQLVQSGAEVKKPGSSVKVSCKASGYSFTGYYMHWVRQAPGQGL |
| | EWIGRVNPNNGGTSYNQKFQGRVTITADESTSTAYMELSSLRSEDTA |
| | VYYCVLTGGYFDYWGQGTLVTVSS (SEQ ID NO: 1492) |
| 8C3V3-30 | QVQLVESGGGVVQPGRSLRLSCAASGYSFTGYYMHWVRQAPGKGL |
| | EWIGRVNPNNGGTSYNDSVKGRFTISRDNSKNTLYLQMNSLRAEDTA |
| | VYYCVLTGGYFDYWGQGTLVTVSS (SEQ ID NO: 1493) |
| 8C3V3-48 | EVQLVESGGGLVQPGGSLRLSCAASGYSFTGYYMEIWVRQAPGKGLE |
| | WIGRVNPNNGGTSYNDSVKGRFTISRDNAKNSLYLQMNSLRAEDTA |
| | VYYCVLTGGYFDYWGQGTLVTVSS (SEQ ID NO: 1494) |
| 8C3V3-7 | EVQLVESGGGLVQPGGSLRLSCAASGYSFTGYYMEIWVRQAPGKGLE |
| | WIGRVNPNNGGTSYNDSVKGRFTISRDNAKNSLYLQMNSLRAEDTA |
| | VYYCVLTGGYFDYWGQGTLVTVSS (SEQ ID NO: 1495) |
| 8C3V4-59 | QVQLQESGPGLVKPSETLSLTCTVSGYSFTGYYMEIWIRQPPGKGLEW |
| | IGRVNPNNGGTSYNPSLKSRVTISVDTSKNQFSLKLSSVTAADTAVYY |
| | CVLTGGYFDYWGQGTLVTVSS (SEQ ID NO: 1496) |
| 8C3V3-15 | EVQLVESGGGLVKPGGSLRLSCAASGYSFTGYYMEIWVRQAPGKGLE |
| | WIGRVNPNNGGTSYNAPVKGRFTISRDDSKNTLYLQMNSLKTEDTAV |
| | YYCVLTGGYFDYWGQGTLVTVSS (SEQ ID NO: 1497) |
| 8C3V4-39 | QLQLQESGPGLVKPSETLSLTCTVSGYSFTGYYMEIWIRQPPGKGLEW |
| | IGRVNPNNGGTSYNPSLKSRVTISVDTSKNQFSLKLSSVTAADTAVYY |
| | CVLTGGYFDYWGQGTLVTVSS (SEQ ID NO: 1498) |

In some embodiments, anti-TREM2 antibodies of the present disclosure comprise a light chain variable region of any one of the antibodies listed in TABLES D1-D6, or selected from 1A7, 3A2, 3B10, 6G12, 6H6, 7A9, 7B3, 8A1, 8E10, 8F11, 8F8, 9F5, 9F5v2, 9G1, 9G3, 10A9, 1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7E5v2, 7F8, 11H5, 7C5, 4F11, 12D9, 1B4v1, 1B4V2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2; and/or a heavy chain variable region of any one of the antibodies listed in TABLES D1-D6, or selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4V2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2.

In some embodiments, the anti-TREM2 antibody is an anti-TREM2 monoclonal antibody selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4V2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2, and humanized variants thereof.

In some embodiments, each of the light chain variable regions disclosed in listed in TABLES D1-D6, or selected from 1A7, 3A2, 3B10, 6G12, 6H6, 7A9, 7B3, 8A1, 8E10, 8F11, 8F8, 9F5, 9F5v2, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7E5v2, 7F8, 11H5, 7C5, 4F11, 12D9, 1B4v1, 1B4V2, 6H2, 7B11v1, 7B 11v2, 18D8, 18E4v1, 18E4v2, 29F6v1, 29F6v2, 40D5v1, 40D5v2, 43B9, 44A8v1, 44A8v2, 44B4v1, and 44B4v2; and/or each of the heavy chain variable region of any one of the antibodies listed in TABLES D1-D6, or selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4V2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2 may be attached to the light chain constant regions (TABLE EN1) and heavy chain constant regions (TABLE EN2) to form complete antibody light and heavy chains, respectively, as further discussed below. Further, each of the generated heavy and light chain sequences may be combined to form a complete antibody structure. It should be understood that the heavy chain and light chain variable regions provided herein can also be attached to other constant domains having different sequences than the exemplary sequences listed herein.

E. PCT Patent Application Publication No. WO2019/028292A1

In some embodiments, the TREM2 agonist is an antibody, or antigen binding fragment thereof, as described in PCT Patent Application Publication No. WO2019/028292A1 (“the '292 application”), which is incorporated by reference herein, in its entirety.

In some embodiments, the TREM2 binding agent comprises an antibody that comprises a light chain variable domain comprising a CDRL1, CDRL2, and CDRL3 (also referred to as HVR-L1, HVR-L2, and HVR-L3, respectively), and a heavy chain variable domain comprising a CDRH1, CDRH2, and CDRH3 (also referred to as HVR-H1, HVR-H2, and HVR-H3, respectively) disclosed in the '573 application specification. In some embodiments, the TREM2 binding agent comprises an antibody that comprises a light chain variable domain and a heavy chain variable domain disclosed in the '573 application specification.

In some embodiments, anti-TREM2 antibodies of the present disclosure bind both human and cynomolgus monkey TREM2 with an affinity that is at least about 1-fold higher than an anti-TREM2 antibody selected from anti-TREM2 antibody comprising a heavy chain variable region comprising the amino acid sequence of SEQ ID NO: 1734 and a light chain variable region comprising the amino acid sequence of SEQ ID NO: 1763 (e.g., antibody AL2p-h50); an anti-TREM2 antibody comprising a heavy chain variable region comprising the amino acid sequence of SEQ ID NO: 1798 and a light chain variable region comprising the amino acid sequence of SEQ ID NO:1810 (e.g., antibody AL2p-h77); and an anti-TREM2 antibody comprising a heavy chain variable region comprising the amino acid sequence of SEQ ID NO: 1826 and a light chain variable region comprising the amino acid sequence of SEQ ID NO: 1827 (e.g., antibody AL2). In some embodiments, anti-TREM2 antibodies of the present disclosure bind to primary human immune cells with an affinity that is at least about 10 times higher than that of an anti-TREM2 antibody selected from an anti-TREM2 antibody comprising a heavy chain variable region comprising the amino acid sequence of SEQ ID NO: 1734 and a light chain variable region comprising the amino acid sequence of SEQ ID NO: 1763; an anti-TREM2 antibody comprising a heavy chain variable region comprising the amino acid sequence of SEQ ID NO: 1798 and a light chain variable region comprising the amino acid sequence of SEQ ID NO:1810; and an anti-TREM2 antibody comprising a heavy chain variable region comprising the amino acid sequence of SEQ ID NO: 1826 and a light chain variable region comprising the amino acid sequence of SEQ ID NO: 1827. In some embodiments, anti-TREM2 antibodies of the present disclosure cluster and activate TREM2 signaling in an amount that is at least about 1-fold greater than that of an anti-TREM2 antibody selected from an anti-TREM2 antibody comprising a heavy chain variable region comprising the amino acid sequence of SEQ ID NO:1734 and a light chain variable region comprising the amino acid sequence of SEQ ID NO: 1763; an anti-TREM2 antibody comprising a heavy chain variable region comprising the amino acid sequence of SEQ ID NO: 1798 and a light chain variable region comprising the amino acid sequence of SEQ ID NO:1810; and an anti-TREM2 antibody comprising a heavy chain variable region comprising the amino acid sequence of SEQ ID NO: 1826 and a light chain variable region comprising the amino acid sequence of SEQ ID NO: 1827. In some embodiments, anti-TREM2 antibodies of the present disclosure increase immune cell survival in vitro that to an extent that is greater than an anti-TREM2 antibody selected from an anti-TREM2 antibody comprising a heavy chain variable region comprising the amino acid sequence of SEQ ID NO: 1734 and a light chain variable region comprising the amino acid sequence of SEQ ID NO: 1763; an anti-TREM2 antibody comprising a heavy chain variable region comprising the amino acid sequence of SEQ ID NO: 1798 and a light chain variable region comprising the amino acid sequence of SEQ ID NO:1810; and an anti-TREM2 antibody comprising a heavy chain variable region comprising the amino acid sequence of SEQ ID NO: 1826 and a light chain variable region comprising the amino acid sequence of SEQ ID NO: 1827. In some embodiments, anti-TREM2 antibodies of the present disclosure may also have improved in vivo half-lives. In some embodiments, anti-TREM2 antibodies of the present disclosure may also decreases plasma levels of soluble TREM2 in vivo. In some embodiments, anti-TREM2 antibodies of the present disclosure may also decrease soluble TREM2. In some embodiments, the soluble TREM2 is decreased about any of 10, 20, 30, 40, 50 or 60%.

In some embodiments, the antibody binds to a TREM2 protein, wherein the antibody comprises a heavy chain variable region and a light chain variable region, wherein the heavy chain variable region comprises: an HVR-H1 comprising the sequence according to Formula I: YAFX₁X₂X₃WMN, wherein X₁ is S or W, X₂ is S, L, or R. and X₃ is S, D, H, Q, or E (SEQ ID NO: 1828); an HVR-H2 comprising the sequence according to Formula II: RIYPGX₁GX₂TNYAX₃KX₄X₅G, wherein X₁ is D, G, E, Q, or V, X₂ is D or Q, X₃ is Q, R, H, W, Y, or G, X₄ is F, R, or W, and X₅ is Q, R, K, or H (SEQ ID NO: 1829); and an HVR-H3 comprising the sequence according to Formula III: ARLLRNX₁PGX₂SYAX₃DY, wherein X₁ is Q or K, X₂ is E, S, or A, and X₃ is M or H (SEQ ID NO: 1830), and wherein the antibody is not an antibody comprising a heavy chain variable region comprising an HVR-H1 comprising the sequence of YAFSSSWMN (SEQ ID NO:1831), an HVR-H2 comprising the sequence of RIYPGDGDTNYAQKFQG (SEQ ID NO: 1832), and an HVR-H3 comprising the sequence of ARLLRNQPGESYAMDY (SEQ ID NO: 1833). In some embodiments, the TREM2 agonist is an antibody that binds to a TREM2 protein, wherein the antibody comprises a heavy chain variable region and a light chain variable region, wherein the light chain variable region comprises: an HVR-L1 comprising the sequence according to Formula IV: RX₁SX₂SLX₃HSNX₄YTYLH, wherein X₁ is S or T, X₂ is Q, R, or S, X₃ is V or I, and. X₄ is G, R, W, Q, or A (SEQ ID NO: 1834); an HVR-L2 comprising the sequence according to Formula V: KVSNRX₁S, wherein X) is F, R, V, or K (SEQ ID NO:1835); and an HVR-L3 comprising the sequence according to Formula V: SQSTRVPYT (SEQ ID NO: 1836), and wherein the antibody is not an antibody comprising a light chain variable region comprising an HVR-L1 comprising the sequence of RSSQSLVHSNGYTYLH (SEQ ID NO: 1837), an HVR-L2 comprising the sequence of KVSNRFS (SEQ ID NO: 1838), and an HVR-L3 comprising the sequence of SQSTRVPYT (SEQ ID NO: 1836). In some embodiments, the antibody comprises a heavy chain variable region and a light chain variable region, wherein the heavy chain variable region comprises: an HVR-H1 comprising the sequence according to Formula I: YAFX₁X₂X₃WMN, wherein X₁ is S or W, X₂ is S, L, or R, and X₃ is S, D, H, Q, or E (SEQ ID NO:1828); an HVR-H2 comprising the sequence according to Formula II: RIYPGX₁GX₂TNYAX₃KX₄X₅G, wherein X₁ is D, G, E, Q, or V, X₂ is D or Q, X₃ is Q, R, H, W, Y, or G, X₄ is F, R, or W, and X₅ is Q, R, K, or H (SEQ ID NO:1829); and an HVR-H3 comprising the sequence according to Formula III: ARLLRNX₁PGX₂SYAX₃DY, wherein X₁ is Q or K, X₂ is E, S, or A, and X₃ is M or H (SEQ ID NO:1830), and the light chain variable region comprises: an HVR-L1 comprising the sequence according to Formula IV: RX₁SX₂SLX₃HSNX₄YTYLH, wherein X₁ is S or T, X₂ is Q, R, or S, X₃ is V or I, and X₄ is G, R, W, Q, or A (SEQ ID NO:1834); an HVR-L2 comprising the sequence according to Formula V: KVSNRX₁S, wherein X₁ is F, R, V, or K (SEQ ID NO: 1835); and an HVR-L3 comprising the sequence: SQSTRVPYT (SEQ ID NO: 1836), and wherein the antibody is not an antibody comprising a heavy chain variable region comprising an HVR-H1 comprising the sequence of YAFSSSWMN (SEQ ID NO:1831), an HVR-H2 comprising the sequence of RIYPGDGDTNYAQKFQG (SEQ ID NO: 1832), and an HVR-H3 comprising the sequence of ARLLRNQPGESYAMDY (SEQ ID NO:1833), and comprising a light chain variable region comprising an HVR-L1 comprising the sequence of RSSQSLVHSNGYTYLH (SEQ ID NO: 1837), an HVR-L2 comprising the sequence of KVSNRFS (SEQ ID NO: 1838), and an HVR-L3 comprising the sequence of SQSTRVPYT (SEQ ID NO: 1836).

In some embodiments, the antibody binds to a TREM2 protein, wherein the antibody comprises a heavy chain variable region and a light chain variable region, wherein the heavy chain variable region comprises: an HVR-H1 comprising a sequence selected from the group consisting of SEQ ID NOS:1839 and 1843; an HVR-H2 comprising a sequence selected from the group consisting of SEQ ID NOS:1840, 1842, 1844, and 1848; and an HVR-H3 comprising a sequence selected from the group consisting of SEQ ID NOS: 1833 and 1845; and/or the light the light chain variable region comprises: an HVR-L1 comprising a sequence selected from the group consisting of 1837, 1846, 1849, and 1851; an HVR-L2 comprising a sequence selected from the group consisting of SEQ ID NOS: 1838, 1841, and 1847; and an HVR-L3 comprising the sequence of SEQ ID NO: 1836. In some embodiments, the antibody comprises a heavy chain variable region and a light chain variable region, wherein the heavy chain variable region comprises: an HVR-H1 comprising the sequence of SEQ ID NO: 1839; an HVR-H2 comprising a sequence selected from the group consisting of SEQ ID NOS: 1840, 1842, and 1848; and an HVR-H3 comprising the sequence of SEQ ID NO: 1833; and/or the light the light chain variable region comprises: an HVR-L1 comprising a sequence selected from the group consisting of 1837, 1849, and 1851; an HVR-L2 comprising a sequence selected from the group consisting of SEQ ID NOS:1838 and 1841; and an HVR-L3 comprising the sequence of SEQ ID NO:1836.

In some embodiments, the antibody binds to a TREM2 protein, wherein the antibody comprises a heavy chain variable region and a light chain variable region, wherein the heavy chain variable region comprises the HVR-H1, HVR-H2, and HVR-H3 of antibody AL2p-2, AL2p-3, AL2p-4, AL2p-7, AL2p-8, AL2p-9, AL2p-10, AL2p-11, AL2p-12, AL2p-13, AL2p-14, AL2p-15, AL2p-16, AL2p-17, AL2p-18, AL2p-19, AL2p-20, AL2p-21, AL2p-22, AL2p-23, AL2p-24, AL2p-25, AL2p-26, AL2p-27, AL2p-28, AL2p-29, AL2p-30, AL2p-31, AL2p-32, AL2p-35, AL2p-36, AL2p-37, AL2p-38, AL2p-39, AL2p-40, AL2p-41, AL2p-42, AL2p-43, AL2p-44, AL2p-45, AL2p-46, AL2p-47, AL2p-48, AL2p-49, AL2p-50, AL2p-51, AL2p-52, AL2p-53, AL2p-54, AL2p-55, AL2p-56. AL2p-57, AL2p-58. AL2p-59, AL2p-60. AL2p-61, or AL2p-62 (as shown in TABLES E1-E3). In some embodiments, the antibody comprises a heavy chain variable region and a light chain variable region, wherein the light chain variable region comprises the HVR-L1, HVR-L2, and HVR-L3 of antibody AL2p-5, AL2p-6, AL2p-7, AL2p-8, AL2p-9, AL2p-10, AL2p-11, AL2p-12, AL2p-13, AL2p-14, AL2p-15, AL2p-16, AL2p-17, AL2p-18, AL2p-19, AL2p-20, AL2p-21, AL2p-22, AL2p-23, AL2p-24, AL2p-25, AL2p-26, AL2p-27, AL2p-28, AL2p-29, AL2p-30, AL2p-31, AL2p-32, AL2p-33, AL2p-38, AL2p-39, AL2p-40, AL2p-41, AL2p-42, AL2p-43, AL2p-44, AL2p-45, AL2p-46, AL2p-47, AL2p-48, AL2p-49, AL2p-50, AL2p-51, AL2p-52, AL2p-53, AL2p-54, AL2p-55, AL2p-56, AL2p-57, AL2p-58, AL2p-59, AL2p-60, AL2p-61, or AL2p-62 (as shown in TABLES E4-E6). In some embodiments, the antibody comprises a heavy chain variable region and a light chain variable region, wherein the heavy chain variable region comprises the HVR-H I, HVR-H2, and HVR-H3 of antibody AL2p-2, AL2p-3, AL2p-4, AL2p-7, AL2p-8, AL2p-9, AL2p-10, AL2p-11, AL2p-12, AL2p-13, AL2p-14, AL2p-15, AL2p-16, AL2p-17, AL2p-18, AL2p-19, AL2p-20, AL2p-21, AL2p-22, AL2p-23, AL2p-24, AL2p-25, AL2p-26, AL2p-27, AL2p-28, AL2p-29, AL2p-30, AL2p-31, AL2p-32, AL2p-35, AL2p-36, AL2p-37, AL2p-38, AL2p-39, AL2p-40, AL2p-41, AL2p-42, AL2p-43, AL2p-44, AL2p-45, AL2p-46, AL2p-47, AL2p-48, AL2p-49, AL2p-50, AL2p-51, AL2p-52, AL2p-53, AL2p-54, AL2p-55, AL2p-56, AL2p-57, AL2p-58, AL2p-59, AL2p-60, AL2p-61, or AL2p-62 (as shown in TABLES E1-E3); and the light chain variable region comprises the HVR-L1. HVR-L2, and HVR-L3 of antibody AL2p-5, AL2p-6, AL2p-7, AL2p-8, AL2p-9, AL2p-10, AL2p-11, AL2p-12, AL2p-13, AL2p-14, AL2p-15, AL2p-16, AL2p-17, AL2p-18, AL2p-19, AL2p-20, AL2p-21, AL2p-22. AL2p-23, AL2p-24, AL2p-25, AL2p-26, AL2p-27, AL2p-28, AL2p-29, AL2p-30, AL2p-31, AL2p-32, AL2p-33, AL2p-38, AL2p-39, AL2p-40, AL2p-41, AL2p-42, AL2p-43, AL2p-44, AL2p-45 AL2p-46, AL2p-47, AL2p-48, AL2p-49, AL2p-50, AL2p-5I, AL2p-52, AL2p-53, AL2p-54, AL2p-55, AL2p-56, AL2p-57, AL2p-58, AL2p-59, AL2p-60, AL2p-61, or AL2p-62 (as shown in TABLES E4-E6). In some embodiments, the antibody comprises a heavy chain variable region comprising an HVR-H1, HVR-H2, and HVR-H3 and a light chain variable region comprising an HVR-L1, HVR-L2, and HVR-L3, wherein the antibody comprises the HVR-H1, HVR-H2, HVR-H3, HVR-L1, HVR-L2. and HVR-L3 of antibody AL2p-2, AL2p-3, AL2p-4, AL2p-5, AL2p-6, AL2p-7, AL2p-8, AL2p-9, AL2p-10, AL2p-11, AL2p-12, AL2p-13, AL2p-14, AL2p-15, AL2p-16, AL2p-17, AL2p-18, AL2p-19, AL2p-20, AL2p-21, AL2p-22, AL2p-23, AL2p-24, AL2p-25, AL2p-26, AL2p-27, AL2p-28, AL2p-29, AL2p-30, AL2p-31, AL2p-32, AL2p-33, AL2p-35, AL2p-36, AL2p-37, AL2p-38, AL2p-39, AL2p-40, AL2p-41, AL2p-42, AL2p-43. AL2p-44, AL2p-45, AL2p-46, AL2p-47, AL2p-48, AL2p-49, AL2p-50, AL2p-51, AL2p-52, AL2p-53, AL2p-54, AL2p-55, AL2p-56, AL2p-57, AL2p-58, AL2p-59, AL2p-60, AL2p-61, or AL2p-62 (as shown in TABLES E1-E3 and TABLES E4-E6).

In some embodiments, the heavy chain variable region comprises one, two, three or four frame work regions selected from VH FRI, VH FR2, VH FR3, and VH FR4, wherein: the VH FRI comprises a sequence selected from the group consisting of SEQ ID NOS: 1716-1718, the VH FR2 comprises a sequence selected from the group consisting of SEQ ID NOS: 1719 and 1720, the VH FR3 comprises a sequence selected from the group consisting of SEQ ID NOS: 1721 and 1722, and the VH FR4 comprises the sequence of SEQ ID NO: 1723; and/or the light chain variable region comprises one, two, three or four frame work regions selected from VL FRI. VL FR2, VL FR3, and VL FR4, wherein: the VL FRI comprises a sequence selected from the group consisting of SEQ ID NOS:1724-1727, the VL FR2 comprises a sequence selected from the group consisting of SEQ ID NOS:1728 and 1729, the VL FR3 comprises a sequence selected from the group consisting of SEQ ID NOS:1730 and 1731, and the VL FR4 comprises a sequence selected from the group consisting of SEQ ID NOS: 1732 and 1733. In some embodiments, the antibody comprises a heavy chain variable region comprising an amino acid sequence selected from the group consisting of SEQ ID NOS: 1734-1777 and 1798; and/or a light chain variable region comprising an amino acid sequence selected from the group consisting of SEQ ID NOS:1799-1820 and 1825. In some embodiments, the antibody comprises the heavy chain variable region of antibody AL2p-h50, AL2p-2. AL2p-3, AL2p-4, AL2p-5, AL2p-6. AL2p-7, AL2p-8, AL2p-9, AL2p-10, AL2p-11, AL2p-12, AL2p-13, AL2p-14, AL2p-15, AL2p-16, AL2p-17, AL2p-18, AL2p-19, AL2p-20, AL2p-21, AL2p-22, AL2p-23, AL2p-24, AL2p-25, AL2p-26, AL2p-27, AL2p-28, AL2p-29, AL2p-30, AL2p-31, AL2p-32, AL2p-33, AL2p-h77, AL2p-35, AL2p-36, AL2p-37, AL2p-38, AL2p-39, AL2p-40, AL2p-41, AL2p-42, AL2p-43, AL2p-44, AL2p-45, AL2p-46, AL2p-47, AL2p-48, AL2p-49, AL2p-50, AL2p-51, AL2p-52, AL2p-53, AL2p-54, AL2p-55, AL2p-56, AL2p-57, AL2p-58, AL2p-59, AL2p-60, AL2p-61, or AL2p-62 (as shown in TABLE E15); and/or the antibody comprises the light chain variable region of antibody AL2p-h50, AL2p-2, AL2p-3. AL2p-4, AL2p-5, AL2p-6, AL2p-7. AL2p-8. AL2p-9, AL2p-10, AL2p-11, AL2p-12, AL2p-13, AL2p-14, AL2p-15, AL2p-16, AL2p-17, AL2p-18, AL2p-19, AL2p-20, AL2p-21, AL2p-22, AL2p-23, AL2p-24, AL2p-25, AL2p-26, AL2p-27, AL2p-28, AL2p-29, AL2p-30, AL2p-31, AL2p-32, AL2p-33, AL2p-h77, AL2p-35, AL2p-36, AL2p-37, AL2p-38, AL2p-39, AL2p-40, AL2p-41, AL2p-42, AL2p-43, AL2p-44, AL2p-45, AL2p-46, AL2p-47, AL2p-48, AL2p-49, AL2p-50, AL2p-5I, AL2p-52, AL2p-53, AL2p-54, AL2p-55, AL2p-56, AL2p-57, AL2p-58, AL2p-59, AL2p-60, AL2p-61, or AL2p-62 (as shown in TABLE E17). In some embodiments: (a) the HVR-H1 comprises the amino acid sequence YAFSSQWMN (SEQ ID NO: 1839), the HVR-H2 comprises the amino acid sequence RIYPGGGDTNYARKFQG (SEQ ID NO: 1840), the HVR-H3 comprises the amino acid sequence ARLLRNQPGESYAMDY (SEQ ID NO: 1833), the HVR-L1 comprises the amino acid sequence RSSQSLVHSNGYTYLH (SEQ ID NO: 1837), the HVR-L2 comprises the amino acid sequence KVSNRRS (SEQ ID NO:1841), and the HVR-L3 comprises the amino acid sequence SQSTRVPYT (SEQ ID NO: 1836); (b) the HVR-H1 comprises the amino acid sequence YAFSSQWMN (SEQ ID NO: 1839), the HVR-H2 comprises the amino acid sequence RIYPGGGDTNYAGKFQG (SEQ ID NO: 1842), the HVR-H3 comprises the amino acid sequence ARLLRNQPGESYAMDY (SEQ ID NO: 1833), the HVR-L1 comprises the amino acid sequence RSSQSLVHSNGYTYLH (SEQ ID NO: 1837), the HVR-L2 comprises the amino acid sequence KVSNRFS (SEQ ID NO: 1838), and the HVR-L3 comprises the amino acid sequence SQSTRVPYT (SEQ ID NO: 1836); (c) the HVR-H1 comprises the amino acid sequence YAFSSDWMN (SEQ ID NO: 1843), the HVR-H2 comprises the amino acid sequence RIYPGEGDTNYARKFHG (SEQ ID NO: 1844) the HVR-H3 comprises the amino acid sequence ARLLRNKPGESYAMDY (SEQ ID NO:1845) the HVR-L1 comprises the amino acid sequence RTSQSLVHSNAYTYLH (SEQ ID NO:1846), the HVR-L2 comprises the amino acid sequence KVSNRVS (SEQ ID NO: 1847). and the HVR-L3 comprises the amino acid sequence SQSTRVPYT (SEQ ID NO: 1836); (d) the HVR-H1 comprises the amino acid sequence YAFSSQWMN (SEQ ID NO: 1839), the HVR-H2 comprises the amino acid sequence RIYPGEGDTNYARKFQG (SEQ ID NO: 1848), the HVR-H3 comprises the amino acid sequence ARLLRNQPGESYAMDY (SEQ ID NO: 1833), the HVR-L1 comprises the amino acid sequence RSSQSLVHSNQYTYLH (SEQ ID NO: 1849), the HVR-L2 comprises the amino acid sequence KVSNRRS (SEQ ID NO:1841), and the HVR-L3 comprises the amino acid sequence SQSTRVPYT (SEQ ID NO: 1836); (e) the HVR-H1 comprises the amino acid sequence YAFSSQWMN (SEQ ID NO: 1839). the HVR-H2 comprises the amino acid sequence RIYPGEGDTNYAGKFQG (SEQ ID NO:1850). the HVR-H3 comprises the amino acid sequence ARLLRNQPGESYAMDY (SEQ ID NO:1833), the HVR-L1 comprises the amino acid sequence RSSQSLVHSNQYTYLH (SEQ ID NO: 1849), the HVR-L2 comprises the amino acid sequence KVSNRFS (SEQ ID NO: 1838), and the HVR-L3 comprises the amino acid sequence SQSTRVPYT (SEQ ID NO: 1836); (f) the HVR-H1 comprises the amino acid sequence YAFSSQWMN (SEQ ID NO: 1839), the HVR-H2 comprises the amino acid sequence RIYPGGGDTNYAGKFQG (SEQ ID NO: 1842). the HVR-H3 comprises the amino acid sequence ARLLRNQPGESYAMDY (SEQ ID NO: 1833), the HVR-L1 comprises the amino acid sequence RSSQSLVHSNRYTYLH (SEQ ID NO:1851), the HVR-L2 comprises the amino acid sequence KVSNRFS (SEQ ID NO: 1838), and the HVR-L3 comprises the amino acid sequence SQSTRVPYT (SEQ ID NO: 1836); or (g) the HVR-H1 comprises the amino acid sequence YAFSSQWMN (SEQ ID NO: 1839), the HVR-H2 comprises the amino acid sequence RIYPGGGDTNYARKFQG (SEQ ID NO: 1840), the HVR-H3 comprises the amino acid sequence ARLLRNQPGESYAMDY (SEQ ID NO:1833), the HVR-L1 comprises the amino acid sequence RSSQSLVHSNRYTYLH (SEQ ID NO:1851). the HVR-L2 comprises the amino acid sequence KVSNRRS (SEQ ID NO:1841), and the HVR-L3 comprises the amino acid sequence SQSTRVPYT (SEQ ID NO: 1836). In some embodiments, the HVR-H1 comprises the amino acid sequence YAFSSQWMN (SEQ ID NO: 1839), the HVR-H2 comprises the amino acid sequence RIYPGGGDTNYARKFQG (SEQ ID NO: 1840), the HVR-H3 comprises the amino acid sequence ARLLRNQPGESYAMDY (SEQ ID NO: 1833), the HVR-L1 comprises the amino acid sequence RSSQSLVHSNGYTYLH (SEQ ID NO: 1837), the HVR-L2 comprises the amino acid sequence KVSNRRS (SEQ ID NO:1841), and the HVR-L3 comprises the amino acid sequence SQSTRVPYT (SEQ ID NO: 1836). In some embodiments, the HVR-H1 comprises the amino acid sequence YAFSSQWMN (SEQ ID NO: 1839), the HVR-H2 comprises the amino acid sequence RIYPGGGDTNYAGKFQG (SEQ ID NO: 1842), the HVR-H3 comprises the amino acid sequence ARLLRNQPGESYAMDY (SEQ ID NO: 1833), the HVR-L1 comprises the amino acid sequence RSSQSLVHSNGYTYLH (SEQ ID NO: 1837), the HVR-L2 comprises the amino acid sequence KVSNRFS (SEQ ID NO: 1838), and the HVR-L3 comprises the amino acid sequence SQSTRVPYT (SEQ ID NO: 1836). In some embodiments, the HVR-H1 comprises the amino acid sequence YAFSSDWMN (SEQ ID NO: 1843), the HVR-H2 comprises the amino acid sequence RIYPGEGDTNYARKFHG (SEQ ID NO:1844), the HVR-H3 comprises the amino acid sequence ARLLRNKPGESYAMDY (SEQ ID NO: 1845), the HVR-L1 comprises the amino acid sequence RTSQSLVHSNAYTYLH (SEQ ID NO: 1846), the HVR-L2 comprises the amino acid sequence KVSNRVS (SEQ ID NO: 1847), and the HVR-L3 comprises the amino acid sequence SQSTRVPYT (SEQ ID NO: 1836). In some embodiments, the HVR-H1 comprises the amino acid sequence YAFSSQWMN (SEQ ID NO: 1839). the HVR-H2 comprises the amino acid sequence RIYPGEGDTNYARKFQG (SEQ ID NO:1848), the HVR-H3 comprises the amino acid sequence ARLLRNQPGESYAMDY (SEQ ID NO: 1833), the HVR-L1 comprises the amino acid sequence RSSQSLVHSNQYTYLH (SEQ ID NO: 1849), the HVR-L2 comprises the amino acid sequence KVSNRRS (SEQ ID NO:1841), and the HVR-L3 comprises the amino acid sequence SQSTRVPYT (SEQ ID NO: 1836). In some embodiments, the HVR-H1 comprises the amino acid sequence YAFSSQWMN (SEQ ID NO: 1839), the HVR-H2 comprises the amino acid sequence RIYPGEGDTNYAGKFQG (SEQ ID NO: 1850), the HVR-H3 comprises the amino acid sequence ARLLRNQPGESYAMDY (SEQ ID NO: 1833), the HVR-L1 comprises the amino acid sequence RSSQSLVHSNQYTYLH (SEQ ID NO: 1849), the HVR-L2 comprises the amino acid sequence KVSNRFS (SEQ ID NO: 1838), and the HVR-L3 comprises the amino acid sequence SQSTRVPYT (SEQ ID NO: 1836). In some embodiments, the HVR-H1 comprises the amino acid sequence YAFSSQWMN (SEQ ID NO: 1839), the HVR-H2 comprises the amino acid sequence RIYPGGGDTNYAGKFQG (SEQ ID NO: 1842), the HVR-H3 comprises the amino acid sequence ARLLRNQPGESYAMDY (SEQ ID NO: 1833), the HVR-L1 comprises the amino acid sequence RSSQSLVHSNRYTYLH (SEQ ID NO:1851), the HVR-L2 comprises the amino acid sequence KVSNRFS (SEQ ID NO: 1838), and the HVR-L3 comprises the amino acid sequence SQSTRVPYT (SEQ ID NO: 1836). In some embodiments, the HVR-H1 comprises the amino acid sequence YAFSSQWMN (SEQ ID NO: 1839), the HVR-H2 comprises the amino acid sequence RIYPGGGDTNYARKFQG (SEQ ID NO: 1840), the HVR-H3 comprises the amino acid sequence ARLLRNQPGESYAMDY (SEQ ID NO: 1833), the HVR-L1 comprises the amino acid sequence RSSQSLVHSNRYTYLH (SEQ ID NO:1851), the HVR-L2 comprises the amino acid sequence KVSNRRS (SEQ ID NO:1841), and the HVR-L3 comprises the amino acid sequence SQSTRVPYT (SEQ ID NO: 1836). In some embodiments, the HVR-H1 comprises the amino acid sequence YAFSSQWMN (SEQ ID NO: 1839), the HVR-H2 comprises the amino acid sequence RIYPGGGDTNYAGKFQG (SEQ ID NO: 1842), the HVR-H3 comprises the amino acid sequence ARLLRNQPGESYAMDY (SEQ ID NO: 1833), the HVR-L1 comprises the amino acid sequence RSSQSLVHSNRYTYLH (SEQ ID NO:1851), the HVR-L2 comprises the amino acid sequence KVSNRFS (SEQ ID NO: 1838), and the HVR-L3 comprises the amino acid sequence SQSTRVPYT (SEQ ID NO: 1836).

In some embodiments, the antibody comprises a heavy chain variable region and a light chain variable region, wherein the heavy chain variable region comprises Kabat CDRs; and/or the light chain variable region comprises Kabat CDRs. In some embodiments, the heavy chain variable region comprises a CDR-H1 comprising the sequence of SQWMN (SEQ ID NO:1901), a CDR-H2 comprising the sequence of RIYPGGGDTNYAGKFQG (SEQ ID NO: 1842); and a CDR-H3 comprising the sequence of LLRNQPGESYAMDY (SEQ ID NO:1902). In some embodiments, the light chain variable region comprises a CDR-L1 comprising the sequence of RSSQSLVHSNGYTYLH (SEQ ID NO:1837), a CDR-L2 comprising the sequence of KVSNRFS (SEQ ID NO:1838); and a CDR-L3 comprising the sequence of SQSTRVPYT (SEQ ID NO: 1836). In some embodiments, the heavy chain variable region comprises a CDR-H1 comprising the sequence of SQWMN (SEQ ID NO:1901), a CDR-H2 comprising the sequence of RIYPGGGDTNYAGKFQG (SEQ ID NO: 1842); and a CDR-H3 comprising the sequence of LLRNQPGESYAMDY (SEQ ID NO: 1902); and the light chain variable region comprises a CDR-L1 comprising the sequence of RSSQSLVHSNGYTYLH (SEQ ID NO:1837), a CDR-L2 comprising the sequence of KVSNRFS (SEQ ID NO: 1838); and a CDR-L3 comprising the sequence of SQSTRVPYT (SEQ ID NO: 1836).

In some embodiments, the antibody comprises a heavy chain variable region and a light chain variable region, wherein the heavy chain variable region comprises Kabat CDRs; and/or the light chain variable region comprises Kabat CDRs. In some embodiments, the heavy chain variable region comprises a CDR-H1 comprising the sequence of SDWMN (SEQ ID NO: 1903), a CDR-H2 comprising the sequence of RIYPGEGDTNYARKFHG (SEQ ID NO: 1844); and a CDR-H3 comprising the sequence of LLRNKPGESYAMDY (SEQ ID NO:1904). In some embodiments, the light chain variable region comprises a CDR-L1 comprising the sequence of RTSQSLVHSNAYTYLH (SEQ ID NO: 1846), a CDR-L2 comprising the sequence of KVSNRVS (SEQ ID NO: 1847); and a CDR-L3 comprising the sequence of SQSTRVPYT (SEQ ID NO: 1836). In some embodiments, the heavy chain variable region comprises a CDR-HI comprising the sequence of SDWMN (SEQ ID NO: 1903), a CDR-H2 comprising the sequence of RIYPGEGDTNYARKFHG (SEQ ID NO:1844); and a CDR-H3 comprising the sequence of LLRNKPGESYAMDY (SEQ ID NO: 1904); and the light chain variable region comprises a CDR-L1 comprising the sequence of RTSQSLVHSNAYTYLH (SEQ ID NO: 1846), a CDR-L2 comprising the sequence of KVSNRVS (SEQ ID NO: 1847); and a CDR-L3 comprising the sequence of SQSTRVPYT (SEQ ID NO: 1836).

In some embodiments, the antibody comprises a heavy chain variable region and a light chain variable region, wherein the heavy chain variable region comprises Kabat CDRs; and/or the light chain variable region comprises Kabat CDRs. In some embodiments, the heavy chain variable region comprises a CDR-H1 comprising the sequence of SQWMN (SEQ ID NO:1901), a CDR-H2 comprising the sequence of RIYPGGGDTNYAGKFQG (SEQ ID NO: 1842); and a CDR-H3 comprising the sequence of LLRNQPGESYAMDY (SEQ ID NO:1902). In some embodiments, the light chain variable region comprises a CDR-L1 comprising the sequence of RSSQSLVHSNRYTYLH (SEQ ID NO:1851), a CDR-L2 comprising the sequence of KVSNRFS (SEQ ID NO:1838); and a CDR-L3 comprising the sequence of SQSTRVPYT (SEQ ID NO: 1836). In some embodiments, the heavy chain variable region comprises a CDR-H1 comprising the sequence of SQWMN (SEQ ID NO: 1901), a CDR-H2 comprising the sequence of RIYPGGGDTNYAGKFQG (SEQ ID NO: 1842); and a Kabat CDR-H3 comprising the sequence of LLRNQPGESYAMDY (SEQ ID NO: 1902); and the light chain variable region comprises a CDR-L1 comprising the sequence of RSSQSLVHSNRYTYLH (SEQ ID NO:1851), a CDR-L2 comprising the sequence of KVSNRFS (SEQ ID NO: 1838); and a CDR-L3 comprising the sequence of SQSTRVPYT (SEQ ID NO: 1836).

In some embodiments, the antibody comprises a heavy chain variable region and a light chain variable region, wherein the heavy chain variable region comprises Kabat CDRs; and/or the light chain variable region comprises Kabat CDRs. In some embodiments, the heavy chain variable region comprises a CDR-H1 comprising the sequence of SQWMN (SEQ ID NO:1901), a CDR-H2 comprising the sequence of RIYPGGGDTNYARKFQG (SEQ ID NO: 1840); and a CDR-H3 comprising the sequence of LLRNQPGESYAMDY (SEQ ID NO:1902). In some embodiments, the light chain variable region comprises a CDR-L1 comprising the sequence of RSSQSLVHSNRYTYLH (SEQ ID NO:1851), a CDR-L2 comprising the sequence of KVSNRRS (SEQ ID NO:1841); and a CDR-L3 comprising the sequence of SQSTRVPYT (SEQ ID NO: 1836). In some embodiments, the heavy chain variable region comprises a CDR-H1 comprising the sequence of SQWMN (SEQ ID NO: 1901), a CDR-H2 comprising the sequence of RIYPGGGDTNYARKFQG(SEQ ID NO: 1840); and a CDR-H3 comprising the sequence of LLRNQPGESYAMDY (SEQ ID NO: 1902); and the light chain variable region comprises a CDR-L1 comprising the sequence of RSSQSLVHSNRYTYLH (SEQ ID NO:1851), a CDR-L2 comprising the sequence of KVSNRRS (SEQ ID NO:1841); and a CDR-L3 comprising the sequence of SQSTRVPYT (SEQ ID NO: 1836).

In some embodiments, the antibody comprises a heavy chain variable region and a light chain variable region, wherein the heavy chain variable region comprises Kabat CDRs; and/or the light chain variable region comprises Kabat CDRs. In some embodiments, the heavy chain variable region comprises a CDR-H1 comprising the sequence of SQWMN (SEQ ID NO:1901), a CDR-H2 comprising the sequence of RIYPGEGDTNYARKFQG (SEQ ID NO: 1848); and a CDR-H3 comprising the sequence of LLRNQPGESYAMDY (SEQ ID NO:1902). In some embodiments, the light chain variable region comprises a CDR-L1 comprising the sequence of RSSQSLVHSNQYTYLH (SEQ ID NO:1849), a CDR-L2 comprising the sequence of KVSNRRS (SEQ ID NO:1841); and a CDR-L3 comprising the sequence of SQSTRVPYT (SEQ ID NO: 1836). In some embodiments, the heavy chain variable region comprises a CDR-H1 comprising the sequence of SQWMN (SEQ ID NO: 1901), a CDR-H2 comprising the sequence of RIYPGEGDTNYARKFQG (SEQ ID NO:1848); and a CDR-H3 comprising the sequence of LLRNQPGESYAMDY (SEQ ID NO: 1902); and the light chain variable region comprises a CDR-L1 comprising the sequence of RSSQSLVHSNQYTYLH (SEQ ID NO:1849), a CDR-L2 comprising the sequence of KVSNRRS (SEQ ID NO:1841); and a CDR-L3 comprising the sequence of SQSTRVPYT (SEQ ID NO: 1836).

In some embodiments, the antibody comprises a heavy chain variable region comprising an amino acid sequence selected from the group consisting of SEQ ID NOS:1734-1778 and 1798; and/or a light chain variable region comprising an amino acid sequence selected from the group consisting of SEQ ID NOS: 1799-1820 and 1825. In some embodiments, the antibody comprises the heavy chain variable region of antibody AL2p-h50, AL2p-2, AL2p-3, AL2p-4, AL2p-5, AL2p-6, AL2p-7, AL2p-8, AL2p-9, AL2p-I0, AL2p-11, AL2p-12, AL2p-13, AL2p-14, AL2p-15, AL2p-16, AL2p-17, AL2p-18, AL2p-19, AL2p-20, AL2p-21, AL2p-22, AL2p-23, AL2p-24, AL2p-25, AL2p-26, AL2p-27, AL2p-28, AL2p-29, AL2p-30, AL2p-31, AL2p-32, AL2p-33, AL2p-h77, AL2p-35, AL2p-36, AL2p-37, AL2p-38, AL2p-39, AL2p-40, AL2p-41, AL2p-42, AL2p-43, AL2p-44, AL2p-45, AL2p-46, AL2p-47, AL2p-48, AL2p-49, AL2p-50, AL2p-51, AL2p-52, AL2p-53, AL2p-54, AL2p-55, AL2p-56, AL2p-57, AL2p-58, AL2p-59, AL2p-60, AL2p-61, or AL2p-62 (as shown in TABLE E15); and/or the antibody comprises the light chain variable region of antibody AL2p-h50, AL2p-2, AL2p-3, AL2p-4, AL2p-5, AL2p-6, AL2p-7, AL2p-8, AL2p-9, AL2p-10, AL2p-11, AL2p-12, AL2p-13, AL2p-14, AL2p-15, AL2p-16, AL2p-17, AL2p-18, AL2p-19, AL2p-20, AL2p-21, AL2p-22, AL2p-23, AL2p-24, AL2p-25, AL2p-26, AL2p-27, AL2p-28, AL2p-29, AL2p-30, AL2p-31, AL2p-32, AL2p-33, AL2p-h77, AL2p-35, AL2p-36, AL2p-37, AL2p-38, AL2p-39, AL2p-40, AL2p-41, AL2p-42, AL2p-43, AL2p-44, AL2p-45, AL2p-46, AL2p-47, AL2p-48, AL2p-49, AL2p-50, AL2p-51, AL2p-52, AL2p-53, AL2p-54, AL2p-55, AL2p-56, AL2p-57, AL2p-58, AL2p-59, AL2p-60, AL2p-61, or AL2p-62 (as shown in TABLE E17). In some embodiments: (a) the heavy chain variable region comprises the amino acid sequence of SEQ ID NO: 1760, and/or the light chain variable region comprises the amino acid sequence of SEQ ID NO: 1804; (b) the heavy chain variable region comprises the amino acid sequence of SEQ ID NO:1766; and/or the light chain variable region comprises the amino acid sequence of SEQ ID NO: 1811; (c) the heavy chain variable region comprises the amino acid sequence of SEQ ID NO:1771; and/or the light chain variable region comprises the amino acid sequence of SEQ ID NO:1815; (d) the heavy chain variable region comprises the amino acid sequence of SEQ ID NO: 1777; and/or the light chain variable region comprises the amino acid sequence of SEQ ID NO: 1817; (e) the heavy chain variable region comprises the amino acid sequence of SEQ ID NO: 1778; and/or the light chain variable region comprises the amino acid sequence of SEQ ID NO: 1818; (f) the heavy chain variable region comprises the amino acid sequence of SEQ ID NO: 1766; and/or the light chain variable region comprises the amino acid sequence of SEQ ID NO: 1819; or (g) the heavy chain variable region comprises the amino acid sequence of SEQ ID NO: 1760; and/or the light chain variable region comprises the amino acid sequence of SEQ ID NO: 1820. In some embodiments, the antibody comprises an Fc region comprising an amino acid sequence selected from the group consisting of SEQ ID NOS: 1853-1863. In some embodiments, the antibody comprises an Fe region comprising the amino acid sequence of SEQ ID NO:1853. In some embodiments, the antibody comprises an Fc region comprising the amino acid sequence of SEQ ID NO: 1854. In some embodiments, the antibody comprises an Fc region comprising the amino acid sequence of SEQ ID NO: 1855. In some embodiments, the antibody comprises an Fc region comprising the amino acid sequence of SEQ ID NO: 1856. In some embodiments, the antibody comprises an Fc region comprising the amino acid sequence of SEQ ID NO: 1857. In some embodiments, the antibody comprises an Fc region comprising the amino acid sequence of SEQ ID NO: 1858. In some embodiments, the antibody comprises an Fe region comprising the amino acid sequence of SEQ ID NO: 1859. In some embodiments, the antibody comprises an Fc region comprising the amino acid sequence of SEQ ID NO: 1860. In some embodiments, the antibody comprises an Fc region comprising the amino acid sequence of SEQ ID NO: 1861. In some embodiments, the antibody comprises an Fc region comprising the amino acid sequence of SEQ ID NO: 1862. In some embodiments, the antibody comprises an Fc region comprising the amino acid sequence of SEQ ID NO: 1863. In some embodiments, the antibody comprises a heavy chain comprising an amino acid sequence selected from the group consisting of SEQ ID NOS:1905-1920; and/or a light chain comprising an amino acid sequence selected from the group consisting of SEQ ID NOS:1921-1925. In some embodiments, the antibody comprises a heavy chain comprising an amino acid sequence selected from the group consisting of SEQ ID NOS:1905 and 1906; and a light chain comprising the amino acid sequence of SEQ ID NO:1921. In some embodiments, the antibody comprises a heavy chain comprising an amino acid sequence selected from the group consisting of SEQ ID NOS: 1907 and 1908; and a light chain comprising the amino acid sequence of SEQ ID NO:1921. In some embodiments, the antibody comprises a heavy chain comprising an amino acid sequence selected from the group consisting of SEQ ID NOS: 1909 and 1910; and a light chain comprising the amino acid sequence of SEQ ID NO: 1922. In some embodiments, the antibody comprises a heavy chain comprising an amino acid sequence selected from the group consisting of SEQ ID NOS:1911 and 1912; and a light chain comprising the amino acid sequence of SEQ ID NO:1922. In some embodiments, the antibody comprises a heavy chain comprising an amino acid sequence selected from the group consisting of SEQ ID NOS: 1913 and 1914; and a light chain comprising the amino acid sequence of SEQ ID NO: 1923. In some embodiments, the antibody comprises a heavy chain comprising an amino acid sequence selected from the group consisting of SEQ ID NOS:1915 and 1916; and a light chain comprising the amino acid sequence of SEQ ID NO: 1925. in some embodiments, the antibody comprises a heavy chain comprising an amino acid sequence selected from the group consisting of SEQ ID NOS:1917 and 1918; and a light chain comprising the amino acid sequence of SEQ ID NO:1925. In some embodiments, the antibody comprises a heavy chain comprising an amino acid sequence selected from the group consisting of SEQ ID NOS: 1919 and 1920; and a light chain comprising the amino acid sequence of SEQ ID NO: 1924.

In some embodiments, the heavy chain variable region comprises the amino acid sequence of SEQ ID NO: 1760, and/or the light chain variable region comprises the amino acid sequence of SEQ ID NO: 1804. In some embodiments, the heavy chain variable region comprises the amino acid sequence of SEQ ID NO: 1766; and/or the light chain variable region comprises the amino acid sequence of SEQ ID NO:1811. In some embodiments, the heavy chain variable region comprises the amino acid sequence of SEQ ID NO:1771; and/or the light chain variable region comprises the amino acid sequence of SEQ ID NO:1815. In some embodiments, the heavy chain variable region comprises the amino acid sequence of SEQ ID NO: 1777; and/or the light chain variable region comprises the amino acid sequence of SEQ ID NO:1817. In some embodiments, the heavy chain variable region comprises the amino acid sequence of SEQ ID NO: 1778; and/or the light chain variable region comprises the amino acid sequence of SEQ ID NO:1718. In some embodiments, the heavy chain variable region comprises the amino acid sequence of SEQ ID NO: 1766; and/or the light chain variable region comprises the amino acid sequence of SEQ ID NO:1819. In some embodiments, the heavy chain variable region comprises the amino acid sequence of SEQ ID NO: 1760; and/or the light chain variable region comprises the amino acid sequence of SEQ ID NO:1820.

In some embodiments, the antibody comprises a heavy chain variable region comprising an amino acid sequence selected from the group consisting of SEQ ID NOS: 1734, 1763 and 1779-1797; and/or a light chain variable region comprising an amino acid sequence selected from the group consisting of SEQ ID NOS:1799, 1811, and 1821-1824. In some embodiments, the antibody comprises the heavy chain variable region of antibody AL2p-h19, AL2p-h21, AL2p-h22, AL2p-h23, AL2p-h24, AL2p-h25, AL2p-h26, AL2p-h27, AL2p-h28, AL2p-h29, AL2p-h30, AL2p-h31, AL2p-h32, AL2p-h33, AL2p-h34, AL2p-1135, AL2p-h36, AL2p-h42, AL2p-h43, AL2p-h44, AL2p-h47, AL2p-h59, AL2p-h76, or AL2p-h90 (as shown in TABLE E15); and/or the antibody comprises the light chain variable region of antibody AL2p-h19, AL2p-h21, AL2p-h22, AL2p-h23, AL2p-h24, AL2p-h25, AL2p-h26, AL2p-h27, AL2p-h28, AL2p-h29, AL2p-h30, AL2p-h31, AL2p-h32, AL2p-h33, AL2p-h34, AL2p-h35, AL2p-h36, AL2p-h42, AL2p-h43, AL2p-h44, AL2p-h47, AL2p-h59, AL2p-h76, or AL2p-h90 (as shown in TABLE E17).

In some embodiments, the antibody comprises a heavy chain comprising an amino acid sequence selected from the group consisting of SEQ ID NOS: 1905-1920; and/or a light chain comprising an amino acid sequence selected from the group consisting of SEQ ID NOS: 1921-1925. In some embodiments, the antibody comprises a heavy chain comprising an amino acid sequence selected from the group consisting of SEQ ID NOS: 1905 and 1906; and a light chain comprising the amino acid sequence of SEQ ID NO:1921. In some embodiments, the antibody comprises a heavy chain comprising an amino acid sequence selected from the group consisting of SEQ ID NOS: 1907 and 1908; and a light chain comprising the amino acid sequence of SEQ ID NO:1921. In some embodiments, the antibody comprises a heavy chain comprising an amino acid sequence selected from the group consisting of SEQ ID NOS:1909 and 1910; and a light chain comprising the amino acid sequence of SEQ ID NO:1922. In some embodiments, the antibody comprises a heavy chain comprising an amino acid sequence selected from the group consisting of SEQ ID NOS: 1911 and 1912; and a light chain comprising the amino acid sequence of SEQ ID NO: 1922. In some embodiments, the antibody comprises a heavy chain comprising an amino acid sequence selected from the group consisting of SEQ ID NOS:1913 and 1914; and a light chain comprising the amino acid sequence of SEQ ID NO: 1923. In some embodiments, the antibody comprises a heavy chain comprising an amino acid sequence selected from the group consisting of SEQ ID NOS:1915 and 1916; and a light chain comprising the amino acid sequence of SEQ ID NO:1925. In some embodiments, the antibody comprises a heavy chain comprising an amino acid sequence selected from the group consisting of SEQ ID NOS: 1917 and 1918; and a light chain comprising the amino acid sequence of SEQ ID NO: 1925. In some embodiments, the antibody comprises a heavy chain comprising an amino acid sequence selected from the group consisting of SEQ ID NOS:1919 and 1920; and a light chain comprising the amino acid sequence of SEQ ID NO: 1924.

In some embodiments that may be combined with any of the preceding embodiments, the antibody is a bispecific antibody recognizing a first antigen and a second antigen, wherein the first antigen is human TREM2 or a naturally occurring variant thereof, and the second antigen is: (a) an antigen facilitating transport across the blood-brain-barrier; (b) an antigen facilitating transport across the blood-brain-barrier selected from the group consisting of transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopeptide, and ANG1005; (c) a disease-causing agent selected from the group consisting of disease-causing peptides or proteins or, disease-causing nucleic acids, wherein the disease-causing nucleic acids are antisense GGCCCC (G2C4) repeat-expansion RNA, the disease-causing proteins are selected from the group consisting of amyloid beta, oligomeric amyloid beta, amyloid beta plaques, amyloid precursor protein or fragments thereof, Tau, TAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein A1, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides; (d) ligands and/or proteins expressed on immune cells, wherein the ligands and/or proteins selected from the group consisting of CD40, OX40, ICOS, CD28, CD137/4-1BB, CD27, GITR, PD-L1, CTLA-4, PD-L2, PD-1, B7-H3, B7-H4, HVEM, BTLA, KIR, GAL9, TIM3, A2AR, LAG-3, and phosphatidylserine; and (e) a protein, lipid, polysaccharide, or glycolipid expressed on one or more tumor cells. In some embodiments, the antibody binds specifically to both human TREM2 and cynomolgus monkey TREM2. In some embodiments, the antibody has a dissociation constant (K_{D}) for human TREM2 and/or cynomolgus monkey TREM2 that is at least 1-fold lower than an anti-TREM2 antibody comprising a heavy chain variable region comprising the amino acid sequence of SEQ ID NO:1734 and a light chain variable region comprising the amino acid sequence of SEQ ID NO: 1763; or at least 1-fold lower than an anti-TREM2 antibody comprising a heavy chain variable region comprising the amino acid sequence of SEQ ID NO:1798 and a light chain variable region comprising the amino acid sequence of SEQ ID NO:1810. In some embodiments, the antibody has a dissociation constant (K_{D}) for human TREM2 that ranges from about 9 μM to about 100 μM, or less than 100 μM, wherein the K_{D} is determined at a temperature of approximately 25° C. In some embodiments, the antibody has a dissociation constant (K_{D}) for cynomolgus monkey TREM2 that ranges from about 50 nM to about 100 μM, or less than 100 μM, wherein the K_{D} is determined at a temperature of approximately 25° C. In some embodiments, the antibody binds to primary human immune cells with an affinity that is at least 10 times higher than that of an anti-TREM2 antibody comprising a heavy chain variable region comprising the amino acid sequence of SEQ ID NO: 1734 and a light chain variable region comprising the amino acid sequence of SEQ ID NO: 1763; or at least 10 times higher than an anti-TREM2 antibody comprising a heavy chain variable region comprising the amino acid sequence of SEQ ID NO: 1798 and a light chain variable region comprising the amino acid sequence of SEQ ID NO:1810. In some embodiments, the antibody clusters and activates TREM2 signaling in an amount that is at least 1-fold greater than that of an anti-TREM2 antibody comprising a heavy chain variable region comprising the amino acid sequence of SEQ ID NO:1734 and a light chain variable region comprising the amino acid sequence of SEQ ID NO: 1763; or at least 1-fold greater than an anti-TREM2 antibody comprising a heavy chain variable region comprising the amino acid sequence of SEQ ID NO: 1798 and a light chain variable region comprising the amino acid sequence of SEQ ID NO:1810. In some embodiments, the antibody increases immune cell survival in vitro that to an extent that is greater than an anti-TREM2 antibody comprising a heavy chain variable region comprising the amino acid sequence of SEQ ID NO:1734 and a light chain variable region comprising the amino acid sequence of SEQ ID NO: 1763; or that is greater than an anti-TREM2 antibody comprising a heavy chain variable region comprising the amino acid sequence of SEQ ID NO:1798 and a light chain variable region comprising the amino acid sequence of SEQ ID NO:1810. In some embodiments, the antibody has an in vivo half-life that is lower than a human control IgG1 antibody. In some embodiments, the antibody decreases plasma levels of soluble TREM2 in vivo by an amount that is at least 25% greater than that of a human control IgG1 antibody. In some embodiments, the antibody decreases plasma levels of soluble TREM2 in vivo by blocking cleavage, by inhibiting one or more metalloproteases, and/or by inducing internalization. In some embodiments, soluble TREM2 is decreased by about any of 10, 20, 30, 40, or 50%. In some embodiments, the antibody competes with one or more antibodies selected from the group consisting of AL2p-h50, AL2p-2, AL2p-3, AL2p-4, AL2p-5, AL2p-6, AL2p-7, AL2p-8, AL2p-9, AL2p-10, AL2p-11, AL2p-12, AL2p-13, AL2p-14, AL2p-15, AL2p-16, AL2p-17, AL2p-18, AL2p-19, AL2p-20, AL2p-21, AL2p-22, AL2p-23, AL2p-24, AL2p-25, AL2p-26, AL2p-27, AL2p-28, AL2p-29, AL2p-30, AL2p-31, AL2p-32, AL2p-33, AL2p-h77, AL2p-35, AL2p-36, AL2p-37, AL2p-38, AL2p-39, AL2p-40, AL2p-41, AL2p-42, AL2p-43, AL2p-44, AL2p-45, AL2p-46, AL2p-47, AL2p-48, AL2p-49, AL2p-50, AL2p-51, AL2p-52, AL2p-53, AL2p-54, AL2p-55, AL2p-56, AL2p-57, AL2p-58, AL2p-59, AL2p-60, AL2p-61, AL2p-62, AL2p-h19, AL2p-h21, AL2p-h22, AL2p-h23, AL2p-h24, AL2p-h25, AL2p-h26, AL2p-h27, AL2p-h28, AL2p-h29, AL2p-h30, AL2p-h31, AL2p-h32, AL2p-h33, AL2p-h34, AL2p-h35, AL2p-h36, AL2p-h42, AL2p-h43, AL2p-h44, AL2p-h47, AL2p-1159, AL2p-h76, AL2p-h90, and any combination thereof for binding to TREM2. In some embodiments, the antibody binds essentially the same TREM2 epitope as an antibody selected from the group consisting of: AL2p-h50, AL2p-2, AL2p-3, AL2p-4, AL2p-5, AL2p-6, AL2p-7, AL2p-8, AL2p-9, AL2p-10, AL2p-11, AL2p-12, AL2p-13, AL2p-14, AL2p-15, AL2p-16, AL2p-17, AL2p-18, AL2p-19, AL2p-20, AL2p-21, AL2p-22, AL2p-23, AL2p-24, AL2p-25, AL2p-26, AL2p-27, AL2p-28, AL2p-29, AL2p-30, AL2p-31, AL2p-32, AL2p-33, AL2p-h77, AL2p-35, AL2p-36, AL2p-37, AL2p-38, AL2p-39, AL2p-40, AL2p-41, AL2p-42, AL2p-43, AL2p-44, AL2p-45, AL2p-46, AL2p-47, AL2p-48, AL2p-49, AL2p-50, AL2p-51, AL2p-52, AL2p-53, AL2p-54, AL2p-55, AL2p-56, AL2p-57, AL2p-58, AL2p-59, AL2p-60, AL2p-61, AL2p-62, AL2p-h19, AL2p-h21, AL2p-h22, AL2p-h23, AL2p-h24, AL2p-h25, AL2p-h26, AL2p-h27, AL2p-h28, AL2p-h29, AL2p-h30, AL2p-h31, AL2p-h32, AL2p-h33, AL2p-h34, AL2p-h35, AL2p-h36, AL2p-h42, AL2p-h43, AL2p-h44, AL2p-h47, AL2p-h59, AL2p-h76, and AL2p-h90. In some embodiments, the antibody binds to one or more amino acids within amino acid residues 149-157 of SEQ ID NO: 1. In some embodiments, the antibody binds to one or more amino acid residues selected from the group consisting of E151, D152, and E156 of SEQ ID NO: 1.

In some embodiments, the antibody is an antibody disclosed in Tables 2A, 2B, 2C, 3A, 3B, 3C, 4A-4D, 5A-5D, 6A, 6B, 7A or 7B of PCT Patent Application Publication No. WO2019/028292A1, reproduced below as TABLES E1-E18.

TABLE E1
Heavy chain HVR H1 sequences of
anti-TREM2 antibodies

| Ab | HVR H1 |
|---|---|
| AL2p-h50, AL2p-2, AL2p-3, | YAFSSSWMN |
| AL2p-4, AL2p-5, AL2p-6, | (SEQ ID |
| AL2p-33, AL2p-h77, and | NO: 1831) |
| AL2p-36 | |
| AL2p-29, AL2p-30, AL2p-31, | YAFSSQWMN |
| AL2p-37, AL2p-58, AL2p-60, | (SEQ ID |
| AL2p-61, and AL2p-62 | NO: 1839) |
| AL2p-10, AL2p-11, AL2p-45, | YAFSSDWMN |
| AL2p-46, AL2p-47, AL2p-48, | (SEQ ID |
| and AL2p-49 | NO: 1843) |
| AL2p-7 and AL2p-8 | YAFSLSWMN |
| | (SEQ ID |
| | NO: 1864) |
| AL2p-9 | YAFSRSWMN |
| | (SEQ ID |
| | NO: 1865) |
| AL2p-12, AL2p-13, AL2p-14, | YAFSSHWMN |
| AL2p-15, AL2p-16, AL2p-17, | (SEQ ID |
| AL2p-18, AL2p-19, AL2p-20, | NO: 1866) |
| AL2p-21, AL2p-22, AL2p-23, | |
| AL2p-24, AL2p-25,AL2p-26 | |
| AL2p-27, AL2p-28, AL2p-38, | |
| AL2p-39, AL2p-40, AL2p-41, | |
| AL2p-42, AL2p-43, AL2p-44, | |
| AL2p-50, AL2p-51, AL2p-52, | |
| AL2p-53, AL2p-54. AL2p-55, | |
| AL2p-56, AL2p-57, and | |
| AL2p-59 | |
| AL2p-32 | YAFSSEWMN |
| | (SEQ ID |
| | NO: 1867) |
| AL2P-35 | YAFWSSWMN |
| | (SEQ ID |
| | NO: 1868) |
| Formula I | YAFX₁X₂X₃WMN |
| | X₁ is S or W |
| | X₂ is S, L, or R |
| | X₃ is S, D, H, |
| | Q, or E |
| | (SEQ ID |
| | NO: 1828) |

TABLE E2
Heavy chain HVR H2 sequences of
anti-TREM2 antibodies

| Ab | HVR H2 |
|---|---|
| AL2p-h50, AL2p-5, | RIYPGDGDTNYAQKFQG |
| AL2p-6, AL2p-9, | (SEQ ID NO: 1832) |
| AL2p-10, AL2p-14, | |
| AL2p-15, AL2p-29, | |
| AL2p-32, AL2p-33, | |
| AL2p-h77, and | |
| AL2p-35 | |
| AL2p-31 and AL2p-60 | RIYPGGGDTNYARKFQG |
| | (SEQ ID NO: 1840) |
| AL2p-37 and AL2p-58 | RIYPGGGDTNYAGKFQG |
| | (SEQ ID NO: 1842) |
| AL2p47, AL2p-48, | RIYPGEGDTNYARKFHG |
| AL2p-49 | (SEQ ID NO: 1844) |
| AL2p-45, AL2p46, | RIYPGEGDTNYARKFQG |
| and AL2p-61 | (SEQ ID NO: 1848) |
| AL2p-62 | RIYPGEGDTNYAGKFQG |
| | (SEQ ID NO: 1850) |
| AL2p-2 and AL2p-24 | RIYPGGGDTNYAQKFQG |
| | (SEQ ID NO: 1869) |
| AL2p-3 | RIYPGEGDTNYAQKFQG |
| | (SEQ ID NO: 1870) |
| AL2p-4 and AL2p-27 | RIYPGQGDTNYAQKFQG |
| | (SEQ ID NO: 1871) |
| AL2p-7 and AL2p-16 | RIYPGDGDTNYAQKFRG |
| | (SEQ ID NO: 1872) |
| AL2p-8, AL2p-11, | RIYPGDGDTNYARKFQG |
| AL2p-19, AL2p-20, | (SEQ ID NO: 1873) |
| and AL2p-36 | |
| AL2p-12 | RIYPGDGDTNYAHKFQG |
| | (SEQ ID NO: 1874) |
| AL2p-13 | RIYPGDGDTNYAQKFKG |
| | (SEQ ID NO: 1875) |
| AL2p-17 | RIYPGDGDTNYAQKRQG |
| | (SEQ ID NO: 1876) |
| AL2p-18 | RIYPGDGDTNYAQKWQG |
| | (SEQ ID NO: 1877) |
| AL2p-21 and AL2p-30 | RIYPGDGDTNYAWKFQG |
| | (SEQ ID NO: 1878) |
| AL2p-22 | RIYPGDGDTNYAYKFQG |
| | (SEQ ID NO: 1879) |
| AL2p-23 | RIYPGDGQTNYAQKRQG |
| | (SEQ ID NO: 1880) |
| AL2p-25, AL2p-38, | RIYPGGGDTNYAQKFRG |
| AL2p-39, and AL2p-40 | (SEQ ID NO: 1881) |
| AL2p-26 | RIYPGGGDTNYAQKRQG |
| | (SEQ ID NO: 1882) |
| AL2p-28 | RIYPGVGDTNYAQKFQG |
| | (SEQ ID NO: 1883) |
| AL2p-41 and AL2p-42 | RIYPGEGDTNYAQKFRG |
| | (SEQ ID NO: 1884) |
| AL2p-43 and AL2p44 | RIYPGGGDTNYARKFRG |
| | (SEQ ID NO: 1885) |
| AL2p-50, AL2p-51, | RIYPGEGDTNYAQKFHG |
| AL2p-52, AL2p-53, | (SEQ ID NO: 1886) |
| AL2p-54, AL2p-55, | |
| AL2p-56, and AL2p-57 | |
| AL2p-59 | RIYPGEGQTNYAQKRQG |
| | (SEQ ID NO: 1887) |
| Formula II | RIYPGX1GX2TNYAX₃KX₄ |
| | X₅G |
| | X₁ is D, G, E, Q, |
| | or V |
| | X₂ is D or Q |
| | X₁ is Q, R, H, W, |
| | Y, or G |
| | X₄ is F, R, or W |
| | X₅ is Q, R, K, or H |
| | (SEQ ID NO: 1829) |

TABLE E3
Heavy chain HVR H3 sequences of
anti-TREM2 antibodies

| Ab | HVR 113 |
|---|---|
| AL2p-h50, AL2p-2, AL2p-3, | ARLLRNQPGESYAMDY |
| AL2p-4, AL2p-5, AL2p-6, | (SEQ ID NO: |
| AL2p-7, AL2p-10, AL2p-11, | 1833) |
| AL2p-12, AL2p-13, AL2p-14. | |
| AL2p-15,, Al2p-17, AL2p-19, | |
| AL2p-20, AL2p-21, AL2p-22, | |
| AL2p-23, AL2p-24, AL2p-25, | |
| AL2p-26, AL2p-27, AL2p-28, | |
| AL2p-29, AL2p-30. AL2p-31, | |
| AL2p-32, AL2p-33, AL2p-h77, | |
| AL2p-37, AL2p-50, AL2p-51, | |
| AL2p-52, AL2p-53, AL2p-58, | |
| AL2p-59. AL2p-60, AL2p-61, | |
| and AL2p-62 | |
| AL2p-45, AL2p-46, AL2p-47, | ARLLRNKPGESYAMDY |
| AL2p-48, AL2p-49, AL2p-54, | (SEQ ID NO: |
| AL2p-55, AL2p-56, and | 1845) |
| AL2p-57 | |
| AL2p-8 and AL2p-18 | ARLLRNQPGSSYAMDY |
| | (SEQ ID NO: |
| | 1888) |
| AL2p-9, AL2p-16, AL2p-36, | ARLLRNQPGASYAMDY |
| AL2p-38, AL2p-39, AL2p-40, | (SEQ ID NO: |
| AL2p-41, AL2p-42, AL2p-43, | 1889) |
| and AL2p-44 | |
| AL2p-35 | ARLLRNQPGESYAHDY |
| | (SEQ ID NO: |
| | 1890) |
| Formula III | ARLLRNX₁PGX₂ |
| | SYAX₃DY |
| | X₁ is Q or K |
| | X₂ is E, S, or A |
| | X₃ is M or H |
| | (SEQ ID NO: |
| | 1830) |

TABLE E4
Light chain HVR LI sequences of
anti-TREM2 antibodies

| Ab | HVR L1 |
|---|---|
| AL2p-h50, AL2p-2, AL2p-3, | RSSQSLVHSNGYTYLH |
| AL2p-4, AL2p-10, AL2p-12, | (SEQ ID NO: |
| AL2p-31, AL2p-32, AL2p-h77, | 1837) |
| AL2p-35, AL2p-36. and | |
| AL2p-37 | |
| AL2p-45, AL2p-47, AL2p-50. | RTSQSLVHSNAYTYLH |
| AL2p-52, AL2p-55, and | (SEQ ID NO: |
| AL2p-56 | 1846) |
| AL2p-61 and AL2p-62 | RSSQSLVHSNQYTYLH |
| | (SEQ ID NO: 1849 |
| AL2p-5, AL2p-58, and | RSSQSLVHSNRYTYLH |
| AL2p-60 | (SEQ ID NO: |
| | 1851) |
| AL2p-6 | RSSQSLVHSNWYTYLH |
| | (SEQ ID NO: |
| | 1891) |
| AL2p-7, AL2p-8, AL2p-13, | RSSQSLIHSNGYTYLH |
| and AL2p-26 | (SEQ ID NO: |
| | 1892) |
| AL2p-9, AL2p-16, AL2p-18. | RTSQSLVHSNGYTYLH |
| AL2p-20, AL2p-23, AL2p-25, | (SEQ ID NO: |
| AL2p-28, and AL2p-33 | 1893) |
| AL2p-11, AL2p-14, AL2p-17, | RSSRSLVHSNGYTYLH |
| AL2p-19, AL2p-22, AL2p-24, | (SEQ ID NO: |
| AL2p-27. and AL2p-29 | 1894) |
| AL2p-15, AL2p-21, and | RSSSSLVHSNGYTYLH |
| AL2p-30 | (SEQ ID NO: |
| | 1895) |
| AL2p-38 and AL2p-43 | RSSRSLVHSNRYTYLH |
| | (SEQ ID NO: |
| | 1896) |
| AL2p-39 and AL2p-41 | RSSRSLVHSNQYTYLH |
| | (SEQ ID NO: |
| | 1897) |
| AL2p-40, AL2p-42, and | RTSRSLVHSNRYTYLH |
| AL2p-44 | (SEQ ID NO: |
| | 1898) |
| AL2p-46, AL2p-48, AL2p-49, | RTSQSLVHSNQYTYLH |
| AL2p-51, AL2p-53, AL2p-54, | (SEQ ID NO: |
| AL2p-57, and AL2p-59 | 1899) |
| Formula IV | RX₁SX₂SLX₃HSNX₄ |
| | YTYLH |
| | X₁ is S or T |
| | X₂ is Q, R, or S |
| | X₃ is V or I |
| | X₄ is G, R, W, Q |
| | or A |
| | (SEQ ID NO: |
| | 1834) |

TABLE E5
Light chain HVR L2 sequences of
anti-TREM2 antibodies

| Ab | HVR L2 |
|---|---|
| AL2p-h50, AL2p-2, AL2p-3, | KVSNRFS |
| AL2p-4, AL2p-5, AL2p-6, | (SEQ ID |
| AL2p-14, AL2p-24, AL2p-29, | NO: 1838) |
| AL2p-h77, AL2p-35, AL2p-36, | |
| AL2p-37, AL2p-58, and | |
| AL2p-62 | |
| AL2p-7, AL2p 8, AL2p 10, | KVSNRRS |
| AL2p 12, AL2p-13, AL2p 22, | (SEQ ID |
| AL2p-26, AL2p-31, AL2p-32, | NO: 1841) |
| AL2p38, AL2p39. AL2p-40, | |
| AL2p-41, AL2p-42, AL2p-43, | |
| AL2p-44, AL2p-60, and | |
| AL2p-61 | |
| AL2p-9, AL2p-11, AL2p-16, | KVSNRVS |
| AL2p-17, AL2p-18, AL2p-19, | (SEQ ID |
| AL2p-20, AL2p-23, AL2p-25, | NO: 1847) |
| AL2p-27, AL2p-28, AL2p-33, | |
| AL2p-45, AL2p-46, AL2p-47, | |
| AL2p-48, AL2p-49, AL2p-50, | |
| AL2p-51, AL2p-52, AL2p-53, | |
| AL2p-54, AL2p-55, AL2p-56, | |
| AL2p-57, and AL2p-59 | |
| AL2p-15, AL2p-21, and | KVSNRKS |
| AL2p-30 | (SEQ ID |
| | NO: 1900) |
| Formula V | KVSNRX₁S |
| | X₁ is F, |
| | R, V, or |
| | K |
| | (SEQ ID |
| | NO: 1835) |

TABLE E6
Light chain HVR L3 sequences of
anti-TR FM2 antibodies

| Ab | HVR L3 |
|---|---|
| AL2p-h50, AL2p-2, AL2p-3, | SQSTRVPYT |
| AL2p-4, AL2p-5, AL2p-6, | (SEQ ID |
| AL2p-7, AL2p-8, AL2p-9, | NO: 1836) |
| AL2p-10, AL2p-11, AL2p-12, | |
| AL2p-13, AL2p-14, AL2p-15, | |
| AL2p-16, AL2p-17, AL2p-18, | |
| AL2p-19, AL2p-20, AL2p-21, | |
| AL2p-22, AL2p-23, AL2p-24, | |
| AT2p-25, AL2p-26, AL2p-27, | |
| AL2p-28, AL2p-29, AL2p-30, | |
| AL2p-31, AL2p-32, AL2p-33, | |
| AL2p-h77, AL2p-35, AL2p-36, | |
| AL2p-37, AL2p-38, AL2p-39, | |
| AL2p-40, AL2p-41, AL2p-42, | |
| AL2p-43, AL2p-44, AL2p-45, | |
| AL2p-46, AL2p-47, AL2p-48, | |
| AL2p-49, AL2p-50, AL2p-51, | |
| AL2p-52, AL2p-53, AL2p-54, | |
| AL2p-55, AL2p-56, AL2p-57, | |
| AL2p-58, AL2p-59, AL2p-60, | |
| AL2p-61, and AL2p-62 | |

TABLE E7
Heavy chain framework I sequences of
anti-TREM2 antibodies

| Ab | VH FR1 |
|---|---|
| AL2p-h50, AL2p-2, AL2p-3, | QVQLVQSGAE |
| AL2p-4, AL2p-5, AL2p-6, | VKKPGSSVKV |
| AL2p-7, AL2p-8, AL2p-9, | SCKASG |
| AL2p-10, AL2p-11, AL2p-12, | (SEQ ID |
| AL2p-13, AL2p-14, AL2p-15, | NO: 1716) |
| AL2p-16, AL2p-17, AL2p-18, | |
| AL2p-19, AL2p-20, AL2p-21, | |
| AL2p-22, AL2p-23, AL2p-24, | |
| AL2p-25, AL2p-26, AL2p-27, | |
| AL2p-28, AL2p-29, AL2p-30, | |
| AL2p-31, AL2p-32, AL2p-38, | |
| AL2p-39, AL2p-40, AL2p-41, | |
| AL2p-42, AL2p-43, AL2p-44, | |
| AL2p-45, AL2p-46, AL2p-47, | |
| AL2p-48, AL2p-50, AL2p-51, | |
| AL2p-54, AL2p-59, AL2p-60, | |
| and AL2p-61 | |
| AL2p-33. AL2p-49, AL2p-52, | EVQLVQSGAE |
| AL2p-53, AL2p-55, AL2p-56, | VKKPGSSVKV |
| and AL2p-57 | SCKASG |
| | (SEQ ID |
| | NO: 1717) |
| AL2p-h77, AL2p-35, AL2p-36, | QVQLVQSGAE |
| AL2p-37. AL2p-58. and AL2p-62 | VKKPGASVKV |
| | SCKASG |
| | (SEQ ID |
| | NO: 1718) |

TABLE E8
Heavy chain framework 2 sequences of
anti-TREM2 antibodies

| Ab | VH FR2 |
|---|---|
| AL2p-h50, AL2p-2, AL2p-3, AL2p-4, | WVRQAPGQGL |
| AL2p-5, AL2p-6, AL2p-7, AL2p-8, | EWMG |
| AL2p-9, AL2p-10, AL2p-11, AL2p-12, | (SEQ ID |
| AL2p-13, AL2p-14, AL2p-15, AL2p-16, | NO: 1719) |
| AL2p-17, AL2p-18, AL2p-19, AL2p-20, | |
| AL2p-21, AL2p-22, AL2p-23, AL2p-24, | |
| AL2p-25, AL2p-26, AL2p-27, AL2p-28, | |
| AL2p-29, AL2p-30, AL2p-31, AL2p-32, | |
| AL2p-33, AL2p-38, AL2p-39, AL2p-40, | |
| AL2p-41, AL2p-42, AL2p-43, AL2p-44, | |
| AL2p-45, AL2p-46, AL2p-47, AL2p48, | |
| AL2p-49, AL2p-50, AL2p-51, AL2p-52, | |
| AL2p-53, AL2p-54, AL2p-55, AL2p-56, | |
| AL2p-57, AL2p-59, AL2p-60, and | |
| AL2p-61 | |
| AL2p-h77, AL2p-35, AL2p-36, | WVRQAPGQRL |
| AL2p-37, AL2p-58, and AL2-62 | EWIG |
| | (SEQ ID |
| | NO: 1720) |

TABLE E9
Heavy chain framework 3 sequences of
anti-TREM2 antibodies

| Ab | VH FR3 |
|---|---|
| AL2p-h50, AL2p-2, AL2p-3. AL2p-4, | RVTITADEST |
| AL2p-5, AL2p-6, AL2p-7, AL2p-8, | STAYMELSSL |
| AL2p-9, AL2p-10, AL2p-11, AL2p-12, | RSEDTAVYYC |
| AL2p-13, AL2p-14, AL2p-15, AL2p-16, | (SEQ ID |
| AL2p-17, AL2p-18, AL2p-19, AL2p-20, | NO: 1721) |
| AL2p-21, AL2p-22, AL2p-23, AL2p-24, | |
| AL2p-25, AL2p-26, AL2p-27, AL2p-28, | |
| AL2p-29, AL2p-30, AL2p-31, AL2p-32, | |
| AL2p-33, AL2p-38, AL2p-39, AL2p-40, | |
| AL2p-41, AL2p-42, AL2p-43, AL2p-44, | |
| AL2p-45, AL2p-46, AL2p-47, AL2p-48, | |
| AL2p-49, AL2p-50, AL2p-51, AL2p-52, | |
| AL2p-53, AL2p-54, AL2p-55, AL2p-56, | |
| AL2p-57, AL2p-59, AL2p-60, and | |
| AL2p-61 | |
| AL2p-h77, AL2p-35, AL2p-36, AL2p-37- | RVTITADTSA |
| AL2p-58, and AL2p-62 | STAYMELSSL |
| | RSEDTAVYYC |
| | (SEQ ID |
| | NO: 1722) |

TABLE E10
Heavy chain framework 4 sequences of
anti-TREM2 antibodies

| Ab | VH FR4 |
|---|---|
| AL2p-h50, AL2p-2, AL2p-3, AL2p-4, | WGQGTLV |
| AL2p-5, AL2p-6, AL2p-7, AL2p-8, | TVSS |
| AL2p-9, AL2p-10, AL2p-11, AL2p-12, | (SEQ ID |
| AL2p-13, AL2p-14, AL2p-15, AL2p-16, | NO: |
| AL2p-17, AL2p-18, AL2p-19, AL2p-20, | 1723) |
| AL2p-21, AL2p-22, AL2p-23, AL2p-24, | |
| AL2p-25, AL2p-26, AL2p-27, AL2p-28, | |
| AL2p-29, AL2p-30, AL2p-31, AL2p-32, | |
| AL2p-33, AL2p-h77, AL2p-35, AL2p-36, | |
| AL2p-37, AL2p-38, AL2p-39, AL2p-40, | |
| AL2p-41, AL2p-42, AL2p-43, AL2p-44, | |
| AL2p-45, AL2p-46, AL2p-47, AL2p-48, | |
| AL2p-49, AL2p-50, AL2p-51, AL2p-52, | |
| AL2p-53, AL2p-54, AL2p-55, AL2p-56, | |
| AL2p-57, AL2p-58, AL2p-59, AL2p-60. | |
| AL2p-61, and AL2p-62 | |

TABLE E11
Light chain framework 1 sequences of
anti-TREM2 antibodies

| Ab | VL FR1 |
|---|---|
| AL2p-h50, AL2p-2, AL2p-3, AL2p-4, | DVVMTQTPLS |
| AL2p-5, AL2p-6, AL2p-11, AL2p-17, | LSVTPGQPAS |
| AL2p-19, AL2p-45, AL2p-46, AL2p-47, | ISC |
| AL2p-48, AL2p-49, AL2p-50, AL2p-51, | (SEQ ID |
| AL2p-52, AL2p-53, AL2p-54, AL2p-55, | NO: 1724) |
| AL2p-56, and AL2p-57 | |
| AL2p-7, AL2p-8, AL2p-9, AL2p-10, | GVVMTQTPLS |
| AL2p-12, AL2p-13, AL2p-14, AL2p-15, | LSVTPGQPAS |
| AL2p-16, AL2p-18, AL2p-20, AL2p-21, | ISC |
| AL2p-22, AL2p-23, AL2p-24, AL2p-25, | (SEQ ID |
| AL2p-26, AL2p-27, AL2p-28, AL2p-29, | NO: 1725) |
| AL2p-30, AL2p-31, AL2p-32, AL2p-38, | |
| AL2p-39, AL2p-40, AL2p-41, AL2p-42, | |
| AL2p-43, AL2p-44, AL2p-59, AL2p-60, | |
| and AL2p-61 | |
| AL2p-33 | GVVMAQTPLS |
| | LSVTPGQPAS |
| | ISC |
| | (SEQ ID |
| | NO: 1726 |
| AL2p-h77, AL2p-35, AL2p-36, AL2p-37, | DVVMTQSPDS |
| AL2p-58, and AL2p-62 | LAVSLGERAT |
| | INC |
| | (SEQ ID |
| | NO: 1727) |

TABLE E12
Light chain framework 2 sequences of
anti-TREM2 antibodies

| Ab | VL FR2 |
|---|---|
| AL2p-h50, AL2p-2, AL2p-3, AL2p-4, | WYLQKPGQSP |
| AL2p-5, AL2p-6, AL2p-7, AL2p-8, | QLLIY |
| AL2p-9, AL2p-10, AL2p-11, AL2p-12, | (SEQ ID |
| AL2p-13, AL2p-14, AL2p-15, AL2p-16, | NO: 1728) |
| AL2p-17, AL2p-18, AL2p-19, AL2p-20, | |
| AL2p-21, AL2p-22, AL2p-23, AL2p-24, | |
| AL2p-25, AL2p-26, AL2p-27, AL2p-28, | |
| AL2p-29, AL2p-30, AL2p-31, AL2p-32, | |
| AL2p-33, AL2p-38, AL2p-39, AL2p-40, | |
| AL2p-41, AL2p-42, AL2p-43, AL2p-44, | |
| AL2p-45, AL2p-46, AL2p-47, AL2p-48, | |
| AL2p-49, AL2p-50, AL2p-51, AL2p-52, | |
| AL2p-53, AL2p-54, AL2p-55, AL2p-56, | |
| AL2p-57, AL2p-59, AL2p-60 and AL2p-61 | |
| AL2p-h77, AL2p-35, AL2p-36, AL2p-37, | WYQQKPGQSP |
| AL2p-58, and AL2p-62 | KLLIY |
| | (SEQ ID |
| | NO: 1729) |

TABLE E13
Light chain framework 3 sequences of
anti-TREM2 antibodies

| Ab | VL FR3 |
|---|---|
| AL2p-h50, AL2p-2, AL2p-3, AL2p-4, | GVPDRFSG |
| AL2p-5, AL2p-6, AL2p-7, AL2p-8, | SGSGTDFT |
| AL2p-9, AL2p-10, AL2p-11. AL2p-12, | LKISRVEA |
| AL2p-13, AL2p-14, AL2p-15, AL2p-16, | EDVGVYYC |
| AL2p-17, AL2p-18, AL2p-19, AL2p-20, | (SEQ ID |
| AL2p-21, AL2p-22, AL2p-23, AL2p-24, | NO: 1730) |
| AL2p-25, AL2p-26, AL2p-27, AL2p-28, | |
| AL2p-29, AL2p-30, AL2p-31, AL2p-32, | |
| AL2p-33, AL2p-38, AL2p-39, AL2p-40, | |
| AL2p-41, AL2p-42, AL2p-43, AL2p-44, | |
| AL2p-45, AL2p-46, AL2p-47, AL2p-48, | |
| AL2p-49, AL2p-50, AL2p-51. AL2p-52, | |
| AL2p-53, AL2p-54, AL2p-55, AL2p-56, | |
| AL2p-57, AL2p-58, AL2p-59, AL2p-60, | |
| and AL2p-61 | |
| AL2p-h77, AL2p-35, AL2p-36, AL2p-37, | GVPDRFSGS |
| and AL2-67 | GSGTDFTLT |
| | ISSLQAEDV |
| | AVYYC |
| | (SEQ ID |
| | NO: 1731) |

TABLE E14
Light chain framework 4 sequences of
anti-TREM2 antibodies

| Ab | VL FR4 |
|---|---|
| AL2p-h50, AL2p-2, AL2p-3, AL2p-4, | FGQGTKLEIK |
| AL2p-5. AT 2p-6. AL2p-7, AL2p-8, | (SEQ ID |
| AL2p-9, AL2p-10, AL2p-11, AL2p-12, | NO: 1732) |
| AL2p-13, AL2p-14, AL2p-15, AL2p-16, | |
| AL2p-17, AL2p-18, AL2p-19, AL2p-20, | |
| AL2p-21, AL2p-22, AL2p-23, AL2p-24, | |
| AL2p-25, AL2p-26, AL2p-27, AL2p-28, | |
| AL2p-29, AL2p-30, AL2p-31, AL2p-32, | |
| AL2p-33, AL2p-38, AL2p-39, AL2p-40, | |
| AL2p-41, AL2p-42, AL2p-43, AL2p-44, | |
| AL2p-45, AL2p-46, AL2p-47, AL2p-48, | |
| AL2p-49, AL2p-50, AL2p-51, AL2p-52, | |
| AL2p-53, AL2p-54, AL2p-55, AL2p-56, | |
| AL2p-57, AL2p-58, AL2p-59, AL2p-60, | |
| and AL2p-61 | |
| AL2p-h77. AL2p-35, AL2p-36, | FGGGTKVEIK |
| AL2p-37, and AL2p-62 | (SEQ ID |
| | NO: 1733 |

TABLE E15
Heavy chain variable region sequences of anti-TREM2 antibodies

| Ab | HCVR |
|---|---|
| AL2p-h50. AL2p-5, | QVQLVQSGAEVKKPGSSVKVSCKASGYAFSSSWMNWVRQAPGQGL |
| and AL2p-6 | EWMGRIYPGDGDTNYAQKFQGRVTITADESTSTAYMELSSLRSEDTA |
| | VYYCARLLRNQPGESYAMDYWGQGTLVTVSS (SEQ ID NO: 1734) |
| AL2p-2 | QVQLVQSGAEVKKPGSSVKVSCKASGYAFSSSWMNWVRQAPGQGLE |
| | WMGRIYPGGGDTNYAQKFQGRVTITADESTSTAYMELSSLRSEDTAV |
| | YYCARLLRNQPGESYAMDYWGQGTLVTVSS (SEQ ID NO: 1735) |
| AL2p-3 | QVQLVQSGAEVKKPGSSVKVSCKASGYAFSSSWMNWVRQAPGQGL |
| | EWMGRIYPGEGDTNYAQKFQGRVTITADESTSTAYMELSSLRSEDTA |
| | VYYCARLLRNQPGESYAMDYWGQGTLVTVSS (SEQ ID NO: 1736) |
| AL2p-4 | QVQLVQSGAEVKKPGSSVKVSCKASGYAFSSSWMNWVRQAPGQGL |
| | EWMGRIYPGQGDTNYAQKFQGRVTITADESTSTAYMELSSLRSEDTA |
| | VYYCARLLRNQPGESYAMDYWGQGTLVTVSS (SEQ ID NO: 1737 |
| AL2p-7 | QVQLVQSGAEVKKPGSSVKVSCKASGYAFSLSWMNWVRQAPGQGL |
| | EWMGRIYPGDGDTNYAQKFRGRVTITADESTSTAYMELSSLRSEDTA |
| | VYYCARLLRNQPGESYAMDYWGQGTLVTVSS (SEQ ID NO: 1738) |
| AL2p-8 | QVQLVQSGAEVKKPGSSVKVSCKASGYAFSLSWMNWVRQAPGQGL |
| | EWMGRIYPGDGDTNYARKFQGRVTITADESTSTAYMELSSLRSEDTA |
| | VYYCARLLRNQPGSSYAMDYWGQGTLVTVSS (SEQ ID NO: 1739) |
| AL2p-9 | QVQLVQSGAEVKKPGSSVKVSCKASGYAFSRSWMNWVRQAPGQGL |
| | EWMGRIYPGDGDTNYAQKFQGRVTITADESTSTAYMELSSLRSEDTA |
| | VYYCARLLRNQPGASYAMDYWGQGTLVTVSS (SEQ ID NO: 1740) |
| AL2p-10 | QVQLVQSGAEVKKPGSSVKVSCKASGYAFSSDWMNWVRQAPGQGL |
| | EWMGRIYPGDGDTNYAQKFQGRVTITADESTSTAYMELSSLRSEDTA |
| | VYYCARLLRNQPGESYAMDYWGQGTLVTVSS (SEQ ID NO: 1741) |
| AL2p-11 | QVQLVQSGAEVKKPGSSVKVSCKASGYAFSSDWMNWVRQAPGQGL |
| | EWMGRIYPGDGDTNYARKFQGRVTITADESTSTAYMELSSLRSEDTA |
| | VYYCARLLRNQPGESYAMDYWGQGTLVTVSS (SEQ ID NO: 1742) |
| AL2p-12 | QVQLVQSGAEVKKPGSSVKVSCKASGYAFSSHWMNWVRQAPGQGL |
| | EWMGRIYPGDGDTNYAHKFQGRVTITADESTSTAYMELSSLRSEDTA |
| | VYYCARLLRNQPGESYAMDYWGQGTLVTVSS (SEQ ID NO: 1743) |
| AL2p-13 | QVQLVQSGAEVKKPGSSVKVSCKASGYAFSSHWMNWVRQAPGQGL |
| | EWMGRIYPGDGDTNYAQKFKGRVTITADESTSTAYMELSSLRSEDTA |
| | VYYCARLLRNQPGESYAMDYWGQGTLVTVSS (SEQ ID NO: 1744) |
| AL2p-14 and AL2p-15 | QVQLVQSGAEVKKPGSSVKVSCKASGYAFSSHWMNWVRQAPGQGL |
| | EWMGRIYPGDGDTNYAQKFQGRVTITADESTSTAYMELSSLRSEDTA |
| | VYYCARLLRNQPGESYAMDYWGQGTLVTVSS (SEQ ID NO: 1745) |
| AL2p-I6 | QVQLVQSGAEVKKPGSSVKVSCKASGYAFSSHWMNWVRQAPGQGL |
| | EWMGRIYPGDGDTNYAQKFRGRVTITADESTSTAYMELSSLRSEDTA |
| | VYYCARLLRNQPGASYAMDYWGQGTLVTVSS (SEQ ID NO: 1746) |
| AL2p-17 | QVQLVQSGAEVKKPGSSVKVSCKASGYAFSSHWMNWVRQAPGQGL |
| | EWMGRIYPGDGDTNYAQKRQGRVTITADESTSTAYMELSSLRSEDTA |
| | VYYCARLLRNQPGESYAMDYWGQGTLVTVSS (SEQ ID NO: 1747) |
| AL2p-18 | QVQLVQSGAEVKKPGSSVKVSCKASGYAFSSHWMNWVRQAPGQGL |
| | EWMGRIYPGDGDTNYAQKWQGRVTITADESTSTAYMELSSLRSEDT |
| | AVYYCARLLRNQPGSSYAMDYWGQGTLVTVSS (SEQ ID NO: 1748) |
| AL2p-19 and AL2p-20 | QVQLVQSGAEVKKPGSSVKVSCKASGYAFSSHWMNWVRQAPGQGL |
| | EWMGRIYPGDGDTNYARKFQGRVTITADESTSTAYMELSSLRSEDTA |
| | VYYCARLLRNQPGESYAMDYWGQGTLVTVSS (SEQ ID NO: 1749) |
| AL2p-21 | QVQLVQSGAEVKKPGSSVKVSCKASGYAFSSHWMNWVRQAPGQGL |
| | EWMGRIYPGDGDTNYAWKFQGRVTITADESTSTAYMELSSLRSEDT |
| | AVYYCARLLRNQPGESYAMDYWGQGTLVTVSS (SEQ ID NO: 1750) |
| AL2p-22 | QVQLVQSGAEVKKPGSSVKVSCKASGYAFSSHWMNWVRQAPGQGL |
| | EWMGRIYPGDGDTNYAYKFQGRVTITADESTSTAYMELSSLRSEDTA |
| | VYYCARLLRNQPGESYAMDYWGQGTLVTVSS (SEQ ID NO: 1751) |
| AL2p-23 | QVQLVQSGAEVKKPGSSVKVSCKASGYAFSSHWMNWVRQAPGQGL |
| | EWMGRIYPGDGQTNYAQKRQGRVTITADESTSTAYMELSSLRSEDTA |
| | VYYCARLLRNQPGESYAMDYWGQGTLVTVSS (SEQ ID NO: 1752) |
| AL2p-24 | QVQLVQSGAEVKKPGSSVKVSCKASGYAFSSHWMNWVRQAPGQGL |
| | EWMGRIYPGGGDTNYAQKFQGRVTITADESTSTAYMELSSLRSEDTA |
| | VYYCARLLRNQPGESYAMDYWGQGTLVTVSS (SEQ ID NO: 1753 |
| AL2p-25 | QVQLVQSGAEVKKPGSSVKVSCKASGYAFSSHWMNWVRQAPGQGL |
| | EWMGRIYPGGGDTNYAQKFRGRVTITADESTSTAYMELSSLRSEDTA |
| | VYYCARLLRNQPGESYAMDYWGQGTLVTVSS (SEQ ID NO: 1754) |
| AL2p-26 | QVQLVQSGAEVKKPGSSVKVSCKASGYAFSSHWMNWVRQAPGQGL |
| | EWMGRIYPGGGDTNYAQKRQGRVTITADESTSTAYMELSSLRSEDTA |
| | VYYCARLLRNQPGESYAMDYWGQGTLVTVSS (SEQ ID NO: 1755) |
| AL2p-27 | QVQLVQSGAEVKKPGSSVKVSCKASGYAFSSHWMNWVRQAPGQGL |
| | EWMGRIYPGQGDTNYAQKFQGRVTITADESTSTAYMELSSLRSEDTA |
| | VYYCARLLRNQPGESYAMDYWGQGTLVTVSS (SEQ ID NO: 1756) |
| AL2p-28 | QVQLVQSGAEVKKPGSSVKVSCKASGYAFSSHWMNWVRQAPGQGL |
| | EWMGRIYPGVGDTNYAQKFQGRVTITADESTSTAYMELSSLRSEDTA |
| | VYYCARLLRNQPGESYAMDYWGQGTLVTVSS (SEQ ID NO: 1757) |
| AL2p-29 | QVQLVQSGAEVKKPGSSVKVSCKASGYAFSSQWMNWVRQAPGQGL |
| | EWMGRIYPGDGDTNYAQKFQGRVTITADESTSTAYMELSSLRSEDTA |
| | VYYCARLLRNQPGESYAMDYWGQGTLVTVSS (SEQ ID NO: 1758) |
| AL2p-30 | QVQLVQSGAEVKKPGSSVKVSCKASGYAFSSQWMNWVRQAPGQGL |
| | EWMGRIYPGDGDTNYAWKFQGRVTITADESTSTAYMELSSLRSEDT |
| | AVYYCARLLRNQPGESYAMDYWGQGTLVTVSS (SEQ ID NO: 1759) |
| AL2p-31, AL2p-60, | QVQLVQSGAEVKKPGSSVKVSCKASGYAFSSQWMNWVRQAPGQGL |
| and AL2p-h31 | EWMGRIYPGGGDTNYARKFQGRVTITADESTSTAYMELSSLRSEDTA |
| | VYYCARLLRNQPGESYAMDYWGQGTLVTVSS (SEQ ID NO: 1760) |
| AL2p-32 | QVQLVQSGAEVKKPGSSVKVSCKASGYAFSSEWMNWVRQAPGQGL |
| | EWMGRIYPGDGDTNYAQKFQGRVTITADESTSTAYMELSSLRSEDTA |
| | VYYCARLLRNQPGESYAMDYWGQGTLVTVSS (SEQ ID NO: 1761) |
| AL2p-33 | EVQLVQSGAEVKKPGSSVKVSCKASGYAFSSSWMNWVRQAPGQGL |
| | EWMGRIYPGDGDTNYAQKFQGRVTITADESTSTAYMELSSLRSEDTA |
| | VYYCARLLRNQPGESYAMDYWGQGTLVTVSS (SEQ ID NO: 1762) |
| AL2p-h77, AL2p-h26, | QVQLVQSGAEVKKPGASVKVSCKASGYAFSSSWMNWVRQAPGQRL |
| and AL2p-h90 | EWIGRIYPGDGDTNYAQKFQGRVTITADTSASTAYMELSSLRSEDTA |
| | VYYCARLLRNQPGESYAMDYWGQGTLVTVSS (SEQ ID NO: 1763) |
| AL2p-35 | QVQLVQSGAEVKKPGASVKVSCKASGYAFWSSWMNWVRQAPGQR |
| | LEWIGRIYPGDGDTNYAQKFQGRVTITADTSASTAYMELSSLRSEDT |
| | AVYYCARLLRNQPGESYAHDYWGQGTLVTVSS (SEQ ID NO: 1764) |
| AL2p-36 | QVQLVQSGAEVKKPGASVKVSCKASGYAFSSSWMNWVRQAPGQRL |
| | EWIGRIYPGDGDTNYARKFQGRVTITADTSASTAYMELSSLRSEDTA |
| | VYYCARLLRNQPGASYAMDYWGQGTLVTVSS (SEQ ID NO: 1765) |
| AL2p-37 and AL2p-58 | QVQLVQSGAEVKKPGASVKVSCKASGYAFSSQWMNWVRQAPGQRL |
| | EWIGRIYPGGGDTNYAGKFQGRVTITADTSASTAYMELSSLRSEDTA |
| | VYYCARLLRNQPGESYAMDYWGQGTLVTVSS (SEQ ID NO: 1766) |
| AL2p-38, AL2p-39, | QVQLVQSGAEVKKPGSSVKVSCKASGYAFSSHWMNWVRQAPGQGL |
| and AL2p-40 | EWMGRIYPGGGDTNYAQKFRGRVTITADESTSTAYMELSSLRSEDTA |
| | VYYCARLLRNQPGASYAMDYWGQGTLVTVSS (SEQ ID NO: 1767) |
| AL2p-41 and AL2p-42 | QVQLVQSGAEVKKPGSSVKVSCKASGYAFSSHWMNWVRQAPGQGL |
| | EWMGRIYPGEGDTNYAQKFRGRVTITADESTSTAYMELSSLRSEDTA |
| | VYYCARLLRNQPGASYAMDYWGQGTLVTVSS (SEQ ID NO: 1768) |
| AL2p-43 and AL2p-44 | QVQLVQSGAEVKKPGSSVKVSCKASGYAFSSHWMNWVRQAPGQGL |
| | EWMGRIYPGGGDTNYARKFRGRVTITADESTSTAYMELSSLRSEDTA |
| | VYYCARLLRNQPGASYAMDYWGQGTLVTVSS (SEQ ID NO: 1769) |
| AL2p-45 and AL2p-46 | QVQLVQSGAEVKKPGSSVKVSCKASGYAFSSDWMNWVRQAPGQGL |
| | EWMGRIYPGEGDTNYARKFQGRVTITADESTSTAYMELSSLRSEDTA |
| | VYYCARLLRNKPGESYAMDYWGQGTLVTVSS (SEQ ID NO: 1770) |
| AL2p-47 and AL2p-48 | QVQLVQSGAEVKKPGSSVKVSCKASGYAFSSDWMNWVRQAPGQGL |
| | EWMGRIYPGEGDTNYARKFHGRVTITADESTSTAYMELSSLRSEDTA |
| | VYYCARLLRNKPGESYAMDYWGQGTLVTVSS (SEQ ID NO: 1771) |
| AL2p-49 | EVQLVQSGAEVKKPGSSVKVSCKASGYAFSSDWMNWVRQAPGQGL |
| | EWMGRIYPGEGDTNYARKFHGRVTITADESTSTAYMELSSLRSEDTA |
| | VYYCARLLRNKPGESYAMDYWGQGTLVTVSS (SEQ ID NO: 1772) |
| AL2p-50 and AL2p-51 | QVQLVQSGAEVKKPGSSVKVSCKASGYAFSSHWMNWVRQAPGQGL |
| | EWMGRIYPGEGDTNYAQKFHGRVTITADESTSTAYMELSSLRSEDTA |
| | VYYCARLLRNQPGESYAMDYWGQGTLVTVSS (SEQ ID NO: 1773) |
| AL2p-52 and AL2p-53 | EVQLVQSGAEVKKPGSSVKVSCKASGYAFSSHWMNWVRQAPGQGL |
| | EWMGRIYPGEGDTNYAQKFHGRVTITADESTSTAYMELSSLRSEDTA |
| | VYYCARLLRNQPGESYAMDYWGQGTLVTVSS (SEQ ID NO: 1774) |
| AL2p-54 | QVQLVQSGAEVKKPGSSVKVSCKASGYAFSSHWMNWVRQAPGQGL |
| | EWMGRIYPGEGDTNYAQKFHGRVTITADESTSTAYMELSSLRSEDTA |
| | VYYCARLLRNKPGESYAMDYWGQGTLVTVSS (SEQ ID NO: 1775) |
| AL2p-55, AL2p-56, | EVQLVQSGAEVKKPGSSVKVSCKASGYAFSSHWMNWVRQAPGQGL |
| and AL2p-57 | EWMGRIYPGEGDTNYAQKFHGRVTITADESTSTAYMELSSLRSEDTA |
| | VYYCARLLRNKPGESYAMDYWGQGTLVTVSS (SEQ ID NO: 1776) |
| AL2p-61 | QVQLVQSGAEVKKPGSSVKVSCKASGYAFSSQWMNWVRQAPGQGL |
| | EWMGRIYPGEGDTNYARKFQGRVTITADESTSTAYMELSSLRSEDTA |
| | VYYCARLLRNQPGESYAMDYWGQGTLVTVSS (SEQ ID NO: 1777) |
| AL2p-62 | QVQLVQSGAEVKKPGASVKVSCKASGYAFSSQWMNWVRQAPGQRL |
| | EWIGRIYPGEGDTNYAGKFQGRVTITADTSASTAYMELSSLRSEDTA |
| | VYYCARLLRNQPGESYAMDYWGQGTLVTVSS (SEQ ID NO: 1778) |
| AL2p-h19 and AL2p- | QVQLVQSGAEVKKPGSSVKVSCKASGYAFSSSWMNWVRQAPGQGL |
| h35 | EWMGRIYPGDGDTNYAQKFQGRATITADTSTSTAYMELSSLRSEDTA |
| | VYYCARLLRNQPGESYAMDYWGQGTLVTVSS (SEQ ID NO: 1779) |
| AL2p-h21 | QVQLVQSGAEVKKPGASVKVSCKASGYAFSSSWMNWVRQAPGQGL |
| | EWMGRIYPGDGDTNYAQKFQGRVTMTRDTSTSTVYMELSSLRSEDT |
| | AVYYCARLLRNQPGESYAMDYWGQGTLVTVSS (SEQ ID NO: 1780) |
| AL2p-h22 | QVQLVQSGAEVKKPGASVKVSCKASGYAFSSSWMNWVRQAPGQGL |
| | EWIGRIYPGDGDTNYAQKFQGRVTMTADTSTSTVYMELSSLRSEDTA |
| | VYYCARLLRNQPGESYAMDYWGQGTLVTVSS (SEQ ID NO: 1781) |
| AL2p-h23 | QVQLVQSGAEVKKPGASLKISCKASGYAFSSSWMNWVRQAPGQGLE |
| | WIGRIYPGDGDTNYAQKFQGRATLTADTSTSTAYMELSSLRSEDTAV |
| | YYCARLLRNQPGESYAMDYWGQGALVTVSS (SEQ ID NO: 1782) |
| AL2p-h24 | QVQLVQSGAEVVKPGASLKISCKASGYAFSSSWMNWVRQAPGQGLE |
| | WIGRIYPGDGDTNYNQKFQGRATLTADTSTSTAYMELSSLRSEDTAV |
| | YFCARLLRNQPGESYAMDYWGQGALVTVSS (SEQ ID NO: 1783) |
| AL2p-h25 | QVQLVQSGAEVKKPGASLKISCKASGYAFSSSWMNWVRQAPGQGLE |
| | WIGRIYPGDGDTNYNGEFRVRATLTADTSTSTAYMELSSLRSEDTAV |
| | YYCARLLRNQPGESYAMDYWGQGALVTVSS (SEQ ID NO: 1784) |
| AL2p-h27 | QVQLVQSGAEVKKPGASVKVSCKASGYAFSSSWMNWVRQAPGQGL |
| | EWIGRIYPGDGDTNYNGEFRVRATLTADTSTSTAYMELSSLRSEDTA |
| | VYFCARLLRNQPGESYAMDYWGQGTLVTVSS (SEQ ID NO: 1785) |
| AL2p-h28 | QVQLVQSGAEVKKPGASVKVSCKASGYAFSSSWMNWVRQAPGQGL |
| | EWIGRIYPGDGDTNYAQKFQGRATLTADTSTSTAYMELSSLRSEDTA |
| | VYFCARLLRNQPGESYAMDYWGQGTLVTVSS (SEQ ID NO: 1786) |
| AL2p-h29 | QVQLVQSGAEVKKPGASVKVSCKASGYAFSSSWMNWVRQAPGQGL |
| | EWIGRIYPGDGDTNYAQKFQGRATMTADTSTSTAYMELSSLRSEDTA |
| | VYYCARLLRNQPGESYAMDYWGQGTLVTVSS (SEQ ID NO: 1787) |
| AL2p-h30 | QVQLVQSGAEVKKPGASVKVSCKASGYAFSSSWMNWVRQAPGQGL |
| | EWMGRIYPGDGDTNYAQKFQGRVTMTADTSTSTAYMELSSLRSEDT |
| | AVYYCARLLRNQPGESYAMDYWGQGTLVTVSS (SEQ ID NO: 1788) |
| AL2p-h32 | QVQLVQSGAEVKKPGSSVKVSCKASGYAFSSSWMNWVRQAPGQGL |
| | EWIGRIYPGDGDTNYNGEFRVRATLTADTSTTTAYMELSSLRSEDTA |
| | VYFCARLLRNQPGESYAMDYWGQGTLVTVSS (SEQ ID NO: 1789) |
| AL2p-h33 | QVQLVQSGAEVKKPGSSVKVSCKASGYAFSSSWMNWVRQAPGQGL |
| | EWIGRIYPGDGDTNYAQKFQGRATLTADTSTTTAYMELSSLRSEDTA |
| | VYFCARLLRNQPGESYAMDYWGQGTLVTVSS (SEQ ID NO: 1790) |
| AL2p-h34 | QVQLVQSGAEVKKPGSSVKVSCKASGYAFSSSWMNWVRQAPGQGL |
| | EWIGRIYPGDGDTNYAQKFQGRATITADTSTSTAYMELSSLRSEDTA |
| | VYFCARLLRNQPGESYAMDYWGQGTLVTVSS (SEQ ID NO: 1791) |
| AL2p-b36 | EVQLLESGGGLVQPGGSLRLSCAASGYAFSSSWMNWVRQAPGKGLE |
| | WIGRIYPGDGDTNYAQKFQGRATISADTSKNTAYLQMNSLRAEDTA |
| | VYYCARLLRNQPGESYAMDYWGQGTLVTVSS (SEQ ID NO: 1792) |
| AL2p-h42 and AL2p- | QVQLVQSGAEVKKPGASVKVSCKASGYAFSSSWMNWVRQAPGQRL |
| h59 | EWMGRIYPGDGDTNYAQKFQGRVTITRDTSASTAYMELSSLRSEDTA |
| | VYYCARLLRNQPGESYAMDYWGQGTLVTVSS (SEQ ID NO: 1793) |
| AL2p-h43 | QVQLVQSGAEVKKPGASLKVSCKASGYAFSSSWMNWVRQAPGQRL |
| | EWIGRIYPGDGDTNYNGEFRVRATLTADTSASTAYMELSSLRSEDTA |
| | VYFCARLLRNQPGESYAMDYWGQGTLVTVSS (SEQ ID NO: 1794) |
| AL2p-h44 | QVQLVQSGAEVKKPGASLKVSCKASGYAFSSSWMNWVRQAPGQRL |
| | EWIGRIYPGDGDTNYAQKFQGRATLTADTSASTAYMELSSLRSEDTA |
| | VYFCARLLRNQPGESYAMDYWGQGTLVTVSS (SEQ ID NO: 1795) |
| AL2p-h47 | QVQLVQSGAEVKKPGASVKVSCKASGYAFSSSWMNWVRQAPGQGL |
| | EWMGRIYPGDGDTNYNGEFRVRVTMTRDTSTSTVYMELSSLRSEDT |
| | AVYYCARLLRNQPGESYAMDYWGQGTLVTVSS (SEQ ID NO: 1796) |
| AL2p-h76 | QVQLVQSGAEVKKPGASVKVSCKASGYAFSSSWMNWVRQAPGQRL |
| | EWIGRIYPGDGDTNYAQKFQGRATITADTSASTAYMELSSLRSEDTA |
| | VYFCARLLRNQPGESYAMDYWGQGTLVTVSS (SEQ ID NO: 1797) |
| AL2p-59 | QVQLVQSGAEVKKPGSSVKVSCKASGYAFSSHWMNWVRQAPGQGL |
| | EWMGRIYPGEGQTNYAQKRQGRVTITADESTSTAYMELSSLRSEDTA |
| | VYYCARLLRNQPGESYAMDYWGQGTLVTVSS (SEQ ID NO: 1798) |

TABLE E16
Heavy chain sequences of anti-TREM2 antibodies

| Ab | HC |
|---|---|
| AL2p-58 huIgG1 | QVQLVQSGAEVKKPGASVKVSCKASGYAFSSQWMNWVRQAPGQRLEWI |
| | GRIYPGGGDTNYAGKFQGRVTITADTSASTAYMELSSLRSEDTAVYYCAR |
| | LLRNQPGESYAMDYWGQGTLVTVSSASTKGPSVFPLAPSSKSTSGGTAAL |
| | GCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFP |
| | PKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYKT |
| | TPPVLDSDGSFFLYSKLTVDKSRWQQGNVFSCSVMHEALFINHYTQKSLSL |
| | SPGK (SEQ ID NO: 1905) |
| AL2p-58 huIgG1 | QVQLVQSGAEVKKPGASVKVSCKASGYAFSSQWMNWVRQAPGQRLEWI |
| | GRIYPGGGDTNYAGKFQGRVTITADTSASTAYMELSSLRSEDTAVYYCAR |
| | LLRNQPGESYAMDYWGQGTLVTVSSASTKGPSVFPLAPSSKSTSGGTAAL |
| | GCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFP |
| | PKPKDTLMISRTPEVTCVVVDVSHEDPEVDGVEVHNAKTKPRE |
| | EQYNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYKT |
| | TPPVLDSDGSFFLYSKLTVDKSRWQQGNVFSCSVMHEALFINHYTQKSLSL |
| | SPG (SEQ ID NO: 1906) |
| AL2p-58 huIgG1 PSEG | QVQLVQSGAEVKKPGASVKVSCKASGYAFSSQWMNWVRQAPGQRLEWI |
| | GRIYPGGGDTNYAGKFQGRVTITADTSASTAYMELSSLRSEDTAVYYCAR |
| | LLRNQPGESYAMDYWGQGTLVTVSSASTKGPSVFPLAPSSKSTSGGTAAL |
| | GCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFP |
| | PKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQP |
| | REPQVYTLPPSRDELTKNQVSLTCLVKGFYPSDIAVEWESNGQPENNYKT |
| | TPPVLD SDGSFFLYSKLTVDKSRWQQGNVFSCSVMHGALFINHYTQKSLSL |
| | SPGK (SEQ ID NO: 1907) |
| AL2p-58 huIgG1 PSEG | QVQLVQSGAEVKKPGASVKVSCKASGYAFSSQWMNWVRQAPGQRLEWI |
| | GRIYPGGGDTNYAGKFQGRVTITADTSASTAYMELSSLRSEDTAVYYCAR |
| | LLRNQPGESYAMDYWGQGTLVTVSSASTKGPSVFPLAPSSKSTSGGTAAL |
| | GCLVKDYFPEPVTVSWNSGALTSGVHTFPAVLQSSGLYSSSVVTVPSSSL |
| | GTQTYICNVNHKPSNTKVDKKVEPKSCDKTHTCPPCPAPELLGGPSVFLFP |
| | PKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQP |
| | REPQVYTLPPSRDELTKNQVSLTCLVKGFYPSDIAVEWESNGQPENNYKT |
| | TPPVLD SDGSFFLYSKLTVDKSRWQQGNVFSCSVMHGALHNHYTQKSLSL |
| | SPG (SEQ ID NO: 1908) |
| AL2p-47 huIgG1 | QVQLVQSGAEVKKPGSSVKVSCKASGYAFSSDWMNWVRQAPGQGLEW |
| | MGRIYPGEGDTNYARKFHGRVTITADESTSTAYMELSSLRSEDTAVYYCA |
| | RLLRNKPGESYAMDYWGQGTLVTVSSASTKGPSVFPLAPSSKSTSGGTAA |
| | LGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLF |
| | PPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQ |
| | PREPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLSL |
| | SPGK (SEQ ID NO: 1909) |
| AL2p-47 hulgG1 | QVQLVQSGAEVKKPGSSVKVSCKASGYAFSSDWMNWVRQAPGQGLEW |
| | MGRIYPGEGDTNYARKFHGRVTITADESTSTAYMELSSLRSEDTAVYYCA |
| | RLLRNKPGESYAMDYWGQGTLVTVSSASTKGPSVFPLAPSSKSTSGGTAA |
| | LGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLF |
| | PPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQ |
| | PREPQVYTLPPSRDELTKNQVSLTCLVKGFYPSDIAVEWESNGQPENNYKT |
| | TPPVLD SDGSFFLYSKLTVDKSRWQQGNVFSCSVMHEALHNHYTQKSLSL |
| | SPG (SEQ ID NO: 1910) |
| AL2p-47 huIgG1 PSEG | QVQLVQSGAEVKKPGSSVKVSCKASGYAFSSDWMNWVRQAPGQGLEW |
| | MGRIYPGEGDTNYARKFHGRVTITADESTSTAYMELSSLRSEDTAVYYCA |
| | RLLRNKPGESYAMDYWGQGTLVTVSSASTKGPSVFPLAPSSKSTSGGTAA |
| | LGCLVKDYFPEPVTVSWNSGALTSGVHTFPAVLQSSGLYSLSSVVTVPSSS |
| | LGTQTYICNVNF1KPSNTKVDKKVEPKSCDKTHTCPPCPAPELLGGPSVFLF |
| | PPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQ |
| | PREPQVYTLPPSRDELTKNQVSLTCLVKGFYPSDIAVEWESNGQPENNYKT |
| | TPPVLDSDGSFFLYSKLTVDKSRWQQGNVFSCSVMHGALFINHYTQKSLSL |
| | SPGK (SEQ ID NO: 1911) |
| AL2p-47 huIgG1 PSEG | QVQLVQSGAEVKKPGSSVKVSCKASGYAFSSDWMNWVRQAPGQGLEW |
| | MGRIYPGEGDTNYARKFHGRVTITADESTSTAYMELSSLRSEDTAVYYCA |
| | RLLRNKPGESYAMDYWGQGTLVTVSSASTKGPSVFPLAPSSKSTSGGTAA |
| | LGCLVKDYFPEPVTVSWNSGALTSGVHTFPAVLQSSGLYSLSSVVTVPSSS |
| | LGTQTYICNVNFIKPSNTKVDKKVEPKSCDKTHTCPPCPAPELLGGPSVFLF |
| | PPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQ |
| | PREPQVYTLPPSRDELTKNQVSLTCLVKGFYPSDIAVEWESNGQPENNYKT |
| | TPPVLDSDGSFFLYSKLTVDKSRWQQGNVFSCSVMHGALFINHYTQKSLSL |
| | SPG (SEQ ID NO: 1912) |
| AL2p-61 hulgG1 | QVQLVQSGAEVKKPGSSVKVSCKASGYAFSSQWMNWVRQAPGQGLEW |
| | MGRIYPGEGDTNYARKFQGRVTITADESTSTAYMELSSLRSEDTAVYYCA |
| | RLLRNQPGESYAMDYWGQGTLVTVSSASTKGPSVFPLAPSSKSTSGGTAA |
| | LGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLFPPK |
| | PKDTLMISRTPEVCVVVDVSHEDPEVKFNWYVDGVEVHNAKTKPREEQYNS |
| | TYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK |
| | (SEQ ID NO: 1913) |
| AL2p-61 guIgG1 | QVQLVQSGAEVKKPGSSVKVSCKASGYAFSSQWMNWVRQAPGQGLEW |
| | MGRIYPGEGDTNYARKFQGRVTITADESTSTAYMELSSLRSEDTAVYYCA |
| | RLLRNQPGESYAMDYWGQGTLVTVSSASTKGPSVFPLAPSSKSTSGGTAA |
| | LGCLVKDYFPEPVTVSWNSGALTSGVHTFPAVLQSSGLYSLSSVVTVPSSS |
| | LGTQTYICNVNFIKPSNTKVDKKVEPKSCDKTHTCPPCPAPELLGGPSVFLF |
| | PPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQ |
| | PREPQVYTLPPSRDELTKNQVSLTCLVKGFYPSDIAVEWESNGQPENNYKT |
| | TPPVLDSDGSFFLYSKLTVDKSRWQQGNVFSCSVMHEALFINHYTQKSLSL |
| | SPG (SEQ ID NO: 1914) |
| AL2p-40 huIgG1 | QVQLVQSGAEVKKPGSSVKVSCKASGYAFSSHWMNWVRQAPGQGLEW |
| | MGRIYPGGGDTNYAQKFRGRVTITADESTSTAYMELSSLRSEDTAVYYCA |
| | RLLRNQPGASYAMDYWGQGTLVTVSSASTKGPSVFPLAPSSKSTSGGTAA |
| | LGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLF |
| | PPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQ |
| | PREPQVYTLPPSRDELTKNQVSLTCLVKGFYPSDIAVEWESNGQPENNYKT |
| | TPPVLDSDGSFFLYSKLTVDKSRWQQGNVFSCSVMHEALFINHYTQKSLSL |
| | SPGK (SEQ ID NO: 1915) |
| AL2p-40 huIgG1 | QVQLVQSGAEVKKPGSSVKVSCKASGYAFSSHWMNWVRQAPGQGLEW |
| | MGRIYPGGGDTNYAQKFRGRVTITADESTSTAYMELSSLRSEDTAVYYCA |
| | RLLRNQPGASYAMDYWGQGTLVTVSSASTKGPSVFPLAPSSKSTSGGTAA |
| | LGCLVKDYFPEPVTVSWNSGALTSGVHTFPAVLQSSGLYSLSSVVTVPSSS |
| | LGTQTYICNVNFIKPSNTKVDKKVEPKSCDKTHTCPPCPAPELLGGPSVFLF |
| | PPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQ |
| | PREPQVYTLPPSRDELTKNQVSLTCLVKGFYPSDIAVEWESNGQPENNYKT |
| | TPPVLDSDGSFFLYSKLTVDKSRWQQGNVFSCSVMHEALFINHYTQKSLSL |
| | SPG (SEQ ID NO: 1916) |
| AL2p-44 huIgG1 | QVQLVQSGAEVKKPGSSVKVSCKASGYAFSSHWMNWVRQAPGQGLEW |
| | MGRIYPGGGDTNYARKFRGRVTITADESTSTAYMELSSLRSEDTAVYYCA |
| | RLLRNQPGASYAMDYWGQGTLVTVSSASTKGPSVFPLAPSSKSTSGGTA |
| | ALGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPELLGGPSVF |
| | LFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAK |
| | GQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALFINHYTQK |
| | SLSLSPGK (SEQ ID NO: 1917) |
| AL2p-44 huIgG1 | QVQLVQSGAEVKKPGSSVKVSCKASGYAFSSHWMNWVRQAPGQGLEW |
| | MGRIYPGGGDTNYARKFRGRVTITADESTSTAYMELSSLRSEDTAVYYCA |
| | RLLRNQPGASYAMDYWGQGTLVTVSSASTKGPSVFPLAPSSKSTSGGTAA |
| | LGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLF |
| | PPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQ |
| | PREPQVYTLPPSRDELTKNQVSLTCLVKGFYPSDIAVEWESNGQPENNYKT |
| | TPPVLDSDGSFFLYSKLTVDKSRWQQGNVFSCSVMHEALFINHYTQKSLSL |
| | SPG (SEQ ID NO: 1918) |
| AL2p-41 huIgG1 | QVQLVQSGAEVKKPGSSVKVSCKASGYAFSSHWMNWVRQAPGQGLEW |
| | MGRIYPGEGDTNYAQKFRGRVTITADESTSTAYMELSSLRSEDTAVYYCA |
| | RLLRNQPGASYAMDYWGQGTLVTVSSASTKGPSVFPLAPSSKSTSGGTAA |
| | LGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLF |
| | PPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQ |
| | PREPQVYTLPPSRDELTKNQVSLTCLVKGFYPSDIAVEWESNGQPENNYKT |
| | TPPVLDSDGSFFLYSKLTVDKSRWQQGNVFSCSVMHEALFINHYTQKSLSL |
| | SPGK (SEQ ID NO: 1919) |
| AL2p-41 huIgG1 | QVQLVQSGAEVKKPGSSVKVSCKASGYAFSSHWMNWVRQAPGQGLEW |
| | MGRIYPGEGDTNYAQKFRGRVTITADESTSTAYMELSSLRSEDTAVYYCA |
| | RLLRNQPGASYAMDYWGQGTLVTVSSASTKGPSVFPLAPSSKSTSGGTAA |
| | LGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLF |
| | PPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQ |
| | PREPQVYTLPPSRDELTKNQVSLTCLVKGFYPSDIAVEWESNGQPENNYKT |
| | TPPVLDSDGSFFLYSKLTVDKSRWQQGNVFSCSVMHEALFINHYTQKSLSL |
| | SPG (SEQ ID NO: 1920) |

TABLE E17
Light chain variable region sequences of anti-TREM2 antibodies

| Ab | LCVR |
|---|---|
| AL2p-h50, AL2p-2, AL2p-3, AL2p- | DVVMTQTPLSLSVTPGQPASISCRSSQSLVHSNGYTYLHWYL |
| 4, AL2p-h42, AL2p-h43, AL2p-h44, | QKPGQSPQLLIYKVSNRFSGVPDRFSGSGSGTDFTLKISRVEA |
| and AL2p-h47 | EDVGVYYCSQSTRVPYTFGQGTKLEIK (SEQ ID NO: 1799) |
| AL2p-5 | DVVMTQTPLSLSVTPGQPASISCRSSQSLVHSNRYTYLHWYL |
| | QKPGQSPQLLIYKVSNRFSGVPDRFSGSGSGTDFTLKISRVEA |
| | EDVGVYYCSQSTRVPYTFGQGTKLEIK (SEQ ID NO: 1800) |
| AL2p-6 | DVVMTQTPLSLSVTPGQPASISCRSSQSLVHSNWYTYLHWYL |
| | QKPGQSPQLLIYKVSNRFSGVPDRFSGSGSGTDFTLKISRVEA |
| | EDVGVYYCSQSTRVPYTFGQGTKLEIK (SEQ ID NO: 1801) |
| AL2p-7, AL2p-8, AL2p-13, and | GVVMTQTPLSLSVTPGQPASISCRSSQSLIHSNGYTYLHWYL |
| AL2p-26 | QKPGQSPQLLIYKVSNRRSGVPDRFSGSGSGTDFTLKISRVEA |
| | EDVGVYYCSQSTRVPYTFGQGTKLEIK (SEQ ID NO: 1802) |
| AL2p-9, AL2p-16, AL2p-18, AL2p- | GVVMTQTPLSLSVTPGQPASISCRTSQSLVHSNGYTYLHWYL |
| 20, AL2p-23, AL2p-25, and AL2p- | QKPGQSPQLLIYKVSNRVSGVPDRFSGSGSGTDFTLKISRVEA |
| 28 | EDVGVYYCSQSTRVPYTFGQGTKLEIK (SEQ ID NO: 1803) |
| AL2p-10, AL2p-12, AL2p-31, and | GVVMTQTPLSLSVTPGQPASISCRSSQSLVHSNGYTYLHWYL |
| AL2p-32 | QKPGQSPQLLIYKVSNRRSGVPDRFSGSGSGTDFTLKISRVEA |
| | EDVGVYYCSQSTRVPYTFGQGTKLEIK (SEQ ID NO: 1804) |
| AL2p-11, AL2p-17, and AL2p-19 | DVVMTQTPLSLSVTPGQPASISCRSSRSLVHSNGYTYLHWYL |
| | QKPGQSPQLLIYKVSNRVSGVPDRFSGSGSGTDFTLKISRVEA |
| | EDVGVYYCSQSTRVPYTFGQGTKLEIK (SEQ ID NO: 1805) |
| AL2p-14, AL2p-24, and AL2p-29 | GVVMTQTPLSLSVTPGQPASISCRSSRSLVHSNGYTYLHWYL |
| | QKPGQSPQLLIYKVSNRFSGVPDRFSGSGSGTDFTLKISRVEAE |
| | DVGVYYCSQSTRVPYTFGQGTKLEIK (SEQ ID NO: 1806) |
| AL2p-15, AL2p-21, and AL2p-30 | GVVMTQTPLSLSVTPGQPASISCRSSSSLVHSNGYTYLHWYL |
| | QKPGQSPQLLIYKVSNRKSGVPDRFSGSGSGTDFTLKISRVEA |
| | EDVGVYYCSQSTRVPYTFGQGTKLEIK (SEQ ID NO: 1807) |
| AL2p-22 | GVVMTQTPLSLSVTPGQPASISCRSSRSLVHSNGYTYLHWYL |
| | QKPGQSPQLLIYKVSNRRSGVPDRFSGSGSGTDFTLKISRVEA |
| | EDVGVYYCSQSTRVPYTFGQGTKLEIK (SEQ ID NO: 1808) |
| AL2p-27 | GVVMTQTPLSLSVTPGQPASISCRSSRSLVHSNGYTYLHWYL |
| | QKPGQSPQLLIYKVSNRVSGVPDRFSGSGSGTDFTLKISRVEA |
| | EDVGVYYCSQSTRVPYTFGQGTKLEIK (SEQ ID NO: 1809) |
| AL2p-33 | GVVMAQTPLSLSVTPGQPASISCRTSQSLVHSNGYTYLHWY |
| | LQKPGQSPQLLIYKVSNRVSGVPDRFSGSGSGTDFTLKISRVE |
| | AEDVGVYYCSQSTRVPYTFGQGTKLEIK (SEQ ID NO: 1810) |
| AL2p-h77, AL2p-35, AL2p-36, | DVVMTQSPDSLAVSLGERATINCRSSQSLVHSNGYTYLHWY |
| AL2p-37, and AL2p-h76 | QQKPGQSPKLLIYKVSNRFSGVPDRFSGSGSGTDFTLTISSLQ |
| | AEDVAVYYCSQSTRVPYTFGGGTKVEIK (SEQ ID NO: 1811) |
| AL2p-38 and AL2p-43 | GVVMTQTPLSLSVTPGQPASISCRSSRSLVHSNRYTYLHWYL |
| | QKPGQSPQLLIYKVSNRRSGVPDRFSGSGSGTDFTLKISRVEA |
| | EDVGVYYCSQSTRVPYTFGQGTKLEIK (SEQ ID NO: 1812) |
| AL2p-39 and AL2p-41 | GVVMTQTPLSLSVTPGQPASISCRSSRSLVHSNQYTYLHWYL |
| | QKPGQSPQLLIYKVSNRRSGVPDRFSGSGSGTDFTLKISRVEA |
| | EDVGVYYCSQSTRVPYTFGQGTKLEIK (SEQ ID NO: 1813) |
| AL2p-40, AL2p-42, and AL2p-44 | GVVMTQTPLSLSVTPGQPASISCRTSRSLVHSNRYTYLHWYL |
| | QKPGQSPQLLIYKVSNRRSGVPDRFSGSGSGTDFTLKISRVEA |
| | EDVGVYYCSQSTRVPYTFGQGTKLEIK (SEQ ID NO: 1814) |
| AL2p-45 AL2p-47 AL2p-50 | DVVMTQTPLSLSVTPGQPASISCRTSQSLVHSNAYTYLHWY |
| AL2p-52, AL2p-55, and AL2p-56 | LQKPGQSPQLLIYKVSNRVSGVPDRFSGSGSGTDFTLKISRVE |
| | AEDVGVYYCSQSTRVPYTFGQGTKLEIK (SEQ ID NO: 1815) |
| AL2p-46, AL2p-48, AL2p-49, | DVVMTQTPLSLSVTPGQPASISCRTSQSLVHSNQYTYLHWY |
| AL2p-51, AL2p-53, AL2p-54, and | LQKPGQSPQLLIYKVSNRVSGVPDRFSGSGSGTDFTLKISRVE |
| AL2p-57 | AEDVGVYYCSQSTRVPYTFGQGTKLEIK (SEQ ID NO: 1816) |
| AL2p-61 | GVVMTQTPLSLSVTPGQPASISCRSSQSLVHSNQYTYLHWYL |
| | QKPGQSPQLLIYKVSNRRSGVPDRFSGSGSGTDFTLKISRVEA |
| | EDVGVYYCSQSTRVPYTFGQGTKLEIK (SEQ ID NO: 1817) |
| AL2p-62 | DVVMTQSPDSLAVSLGERATINCRSSQSLVHSNQYTYLHWY |
| | QQKPGQSPKLLIYKVSNRFSGVPDRFSGSGSGTDFTLTISSLQ |
| | AEDVAVYYCSQSTRVPYTFGGGTKVEIK (SEQ ID NO: 1818) |
| AL2p-58 | DVVMTQSPDSLAVSLGERATINCRSSQSLVHSNRYTYLHWY |
| | QQKPGQSPKLLIYKVSNRFSGVPDRFSGSGSGTDFTLKISRVE |
| | AEDVGVYYCSQSTRVPYTFGQGTKLEIK (SEQ ID NO: 1819) |
| AL2p-60 | GVVMTQTPLSLSVTPGQPASISCRSSQSLVHSNRYTYLHWYL |
| | QKPGQSPQLLIYKVSNRRSGVPDRFSGSGSGTDFTLKISRVEA |
| | EDVGVYYCSQSTRVPYTFGQGTKLEIK (SEQ ID NO: 1820) |
| AL2p-h19 | DIVMTQTPLSLSVTPGQPASISCRSSQSLVHSNGYTYLHWYL |
| | QKPGQSPQLLIYKVSNRFSGVPDRFSGSGSGTDFTLKISRVEA |
| | EDVGVYYCSQSTRVPYTFGQGTKLEIK (SEQ ID NO: 1821) |
| AL2p-h21, AL2p-h22, AL2p-h23, | DVVMTQTPLSLSVTPGQPASISCRSSQSLVHSNGYTYLHWYL |
| AL2p-h24, AL2p-h25, AL2p-h26, | QKPGQSPQLLIYKVSNRFSGVPDRFSGSGSGTDFTLKISRVEA |
| AL2p-h27, AL2p-h28, AL2p-h29 | EDLGVYFCSQSTRVPYTFGQGTKLEIK (SEQ ID NO: 1822) |
| AL2p-h30, AL2p-h31, AL2p-h32, | |
| AL2p-h33, AL2p- h34, AL2p-h35, | |
| AL2p-h36 | DIVMTQSPLSLPVTPGEPASISCRSSQSLVHSNGYTYLHWYL |
| AL2p-h59 | QKPGQSPQLLIYKVSNRFSGVPDRFSGSGSGTDFTLKISRVEA |
| | EDVGVYYCSQSTRVPYTFGGGTKVEIK (SEQ ID NO: 1823) |
| AL2p-h90 | DVQMTQSPSSLSASVGDRVTITCRSSQSLVHSNGYTYLHWY |
| | QQKPGKSPKLLIYKVSNRFSGVPSRFSGSGSGTDFTLTISSLQP |
| | EDFATYYCSQSTRVPYTFGGGTKVEIK (SEQ ID NO: 1824) |
| AL2p-59 | GVVMTQTPLSLSVTPGQPASISCRTSQSLVHSNQYTYLHWY |
| | LQKPGQSPQLLIYKVSNRVSGVPDRFSGSGSGTDFTLKISRVE |
| | AEDVGVYYCSQSTRVPYTFGQGTKLEIK (SEQ ID NO: 1825) |

TABLE E18
Light chain sequences of anti-TREM2 antibodies

| Ab | LC |
|---|---|
| AL2p-58 | DVVMTQSPDSLAVSLGERATINCRSSQSLVHSNRYTYLH |
| huIgGl, | WYQQKPGQSPKLLIYKVSNRFSGVPDRFSGSGSGTDFTL |
| and | KISRVEAEDVGVYYCSQSTRVPYTFGQGTKLEIKRTVAA |
| AL2p-58 | PSVFIFPPSDEQLKSGTASVVCLLNNFYPREAKVQWKVD |
| huIgG1 | NALQSGNSQESVTEQDSKDSTYSLSSTLTLSKADYEKHK |
| PSEG | VYACEVTHQGLSSPVTKSFNRGEC |
| | (SEQ ID NO: 1921) |
| AL2p-47 | DVVMTQTPLSLSVTPGQPASISCRTSQSLVHSNAYTYLH |
| huIgGl, | WYLQKPGQSPQLLIYKVSNRVSGVPDRFSGSGSGTDFTL |
| and | KISRVEAEDVGVYYCSQSTRVPYTFGQGTKLEIKRTVAA |
| AL2p-47 | PSVFIFPPSDEQLKSGTASVVCLLNNFYPREAKVQWKVD |
| huIgG1 | NALQSGNSQESVTEQDSKDSTYSLSSTLTLSKADYEKHK |
| PSEG | VYACEVTHQGLSSPVTKSFNRGEC |
| | (SEQ ID NO: 1922) |
| AL2p-61 | GVVMTQTPLSLSVTPGQPASISCRSSQSLVHSNQYTYLH |
| huIgG1 | WYLQKPGQSPQLLIYKVSNRRSGVPDRFSGSGSGTDFTL |
| | KISRVEAEDVGVYYCSQSTRVPYTFGQGTKLEIKRTVAA |
| | PSVFIFPPSDEQLKSGTASVVCLLNNFYPREAKVQWKVD |
| | NALQSGNSQESVTEQDSKDSTYSLSSTLTLSKADYEKHK |
| | VYACEVTHQGLSSPVTKSFNRGEC |
| | (SEQ ID NO: 1923) |
| AL2p-41 | GVVMTQTPLSLSVTPGQPASISCRSSRSLVHSNQYTYLH |
| huIgG1 | WYLQKPGQSPQLLIYKVSNRRSGVPDRFSGSGSGTDFTL |
| | KISRVEAEDVGVYYCSQSTRVPYTFGQGTKLEIKRTVAA |
| | PSVFIFPPSDEQLKSGTASVVCLLNNFYPREAKVQWKVD |
| | NALQSGNSQESVTEQDSKDSTYSLSSTLTLSKADYEKHK |
| | VYACEVTHQGLSSPVTKSFNRGEC |
| | (SEQ ID NO: 1924) |
| AL2p-40 | GVVMTQTPLSLSVTPGQPASISCRTSRSLVHSNRYTYLH |
| huIgG1, | WYLQKPGQSPQLLIYKVSNRRSGVPDRFSGSGSGTDFTL |
| and | KISRVEAEDVGVYYCSQSTRVPYTFGQGTKLEIKRTVAA |
| AL2p-44 | PSVFIFPPSDEQLKSGTASVVCLLNNFYPREAKVQWKVD |
| huIgG1 | NALQSGNSQESVTEQDSKDSTYSLSSTLTLSKADYEKHK |
| | VYACEVTHQGLSSPVTKSFNRGEC |
| | (SEQ ID NO: 1925) |

In some embodiments, each of the light chain variable regions and each of the heavy chain variable regions disclosed in TABLES E1-E18 as well as specific combinations thereof and other embodiments of the anti-TREM2 antibody described in the '573 application and herein may be attached to the light chain constant regions (TABLE EN1) and heavy chain constant regions (TABLE EN2) to form complete antibody light and heavy chains, respectively, as further discussed below. Further, each of the generated heavy and light chain sequences may be combined to form a complete antibody structure. It should be understood that the heavy chain and light chain variable regions provided herein can also be attached to other constant domains having different sequences than the exemplary sequences listed herein.

F. PCT Patent Application Publication No. WO2018/015573A1

In some embodiments, the TREM2 agonist is an antibody, or antigen binding fragment thereof, that prevents the cleavage of TREM2 as described in PCT Patent Application Publication No. WO2018/015573A1 (“the '573 application”), which is incorporated by reference herein, in its entirety.

In some embodiments, the TREM2 binding agent comprises an antibody that comprises a light chain variable domain comprising a CDRL1, CDRL2, and CDRL3, and a heavy chain variable domain comprising a CDRH1, CDRH2, and CDRH3 disclosed in the '573 application specification. In some embodiments, the TREM2 binding agent comprises an antibody that comprises a light chain variable domain and a heavy chain variable domain disclosed in the '573 application specification.

In some embodiments, the antibody is a binding molecule that inhibits (preferably prevents) TREM2 cleavage. More specifically, in the context of the present invention cleavage (i.e. shedding) of the TREM2 ectodomain is inhibited by the binding molecule of the present invention. In some embodiments, the antibody is a binding molecule that inhibits (preferably prevents) TREM2 cleavage and activates TREM2 activity. In some embodiments, the herein provided binding molecule has a binding site within the ectodomain of TREM2, preferably the stalk region of the TREM2 ectodomain.

In some embodiments, the antibody is:
(1) an antibody, wherein the heavy chain variable region comprises the sequence of SEQ ID NO: 1955 and the light chain variable region comprises the sequence of SEQ ID NO: 1965; and wherein the antibody inhibits TREM2 cleavage;
(2) an antibody, wherein the heavy chain variable region comprises a sequence having at least 85%, preferably at least 90%, more preferably at least 95%, even more preferably at least 98%, and most preferably at least 99% identity to SEQ ID NO: 1955, and the light chain variable region comprises a sequence having at least 85%, preferably at least 90%, more preferably at least 95%, even more preferably at least 98%, and most preferably at least 99% identity to SEQ ID NO: 1965; and wherein the antibody inhibits TREM2 cleavage;
(3) an antibody, wherein the CDR1 of the heavy chain variable region comprises the amino acid sequence of SEQ ID NO: 1975; the CDR2 of the heavy chain variable region comprises the amino acid sequence of SEQ ID NO: 1985; the CDR3 of the heavy chain variable region comprises the amino acid sequence of SEQ ID NO: 1995; the CDR1 of the light chain variable region comprises the amino acid sequence of SEQ ID NO: 2005; the CDR2 of the light chain variable region comprises the amino acid sequence of SEQ ID NO:2015; and the CDR3 of the light chain variable region comprises the amino acid sequence of SEQ ID NO:2025; and wherein the antibody inhibits TREM2 cleavage; or
(4) an antibody, wherein the CDR1 of the heavy chain variable region comprises an amino acid sequence having at least 70%, preferably at least 75%, more preferably at least 80%, and most preferably at least 85% identity to SEQ ID NO: 1975; the CDR2 of the heavy chain variable region comprises an amino acid sequence having at least 70%, preferably at least 75%, more preferably at least 80%, even more preferably at least 85%, and most preferably at least 90% identity to SEQ ID NO:1985; the CDR3 of the heavy chain variable region comprises an amino acid sequence having at least 70%, preferably at least 75%, more preferably at least 80%, even more preferably at least 85%, and most preferably at least 90% identity to SEQ ID NO: 1995; the CDR1 of the light chain variable region comprises an amino acid sequence having at least 70%, preferably at least 75%, more preferably at least 80%, even more preferably at least 85%, and most preferably at least 90% identity to SEQ ID NO:2005; the CDR2 of the light chain variable region comprises an amino acid sequence having at least 60%, preferably 100% identity to SEQ ID NO:2015; and the CDR3 of the light chain variable region comprises an amino acid sequence having at least 70%, preferably at least 75%, more preferably at least 80%, and most preferably at least 85% identity to SEQ ID NO:2025; and wherein the antibody inhibits TREM2 cleavage.

In some embodiments, the antibody is antibody clone 14D3, which is:
(1) an antibody, wherein the heavy chain variable region comprises the sequence of SEQ ID NO: 1946 and the light chain variable region comprises the sequence of SEQ ID NO: 1956; and wherein the antibody inhibits TREM2 cleavage;
(2) an antibody, wherein the heavy chain variable region comprises a sequence having at least 85% identity to SEQ ID NO: 1946, and the light chain variable region comprises a sequence having at least 85% identity to SEQ ID NO: 1956; and wherein the antibody inhibits TREM2 cleavage;
(3) an antibody, wherein the CDR1 of the heavy chain variable region comprises the amino acid sequence of SEQ ID NO: 1966; the CDR2 of the heavy chain variable region comprises the amino acid sequence of SEQ ID NO: 1976; the CDR3 of the heavy chain variable region comprises the amino acid sequence of SEQ ID NO: 1986; the CDR1 of the light chain variable region comprises the amino acid sequence of SEQ ID NO: 1996; the CDR2 of the light chain variable region comprises the amino acid sequence of SEQ ID NO:2006; and the CDR3 of the light chain variable region comprises the amino acid sequence of SEQ ID NO:2016; and wherein the antibody inhibits TREM2 cleavage; or
(4) an antibody, wherein the CDR1 of the heavy chain variable region comprises an amino acid sequence having at least 70% identity to SEQ ID NO: 1966; the CDR2 of the heavy chain variable region comprises an amino acid sequence having at least 70% identity to SEQ ID NO: 1976; the CDR3 of the heavy chain variable region comprises an amino acid sequence having at least 70% identity to SEQ ID NO: 1986; the CDR1 of the light chain variable region comprises an amino acid sequence having at least 70% identity to SEQ ID NO: 1996; the CDR2 of the light chain variable region comprises an amino acid sequence having at least 60% identity to SEQ ID NO:2006; and the CDR3 of the light chain variable region comprises an amino acid sequence having at least 70% identity to SEQ ID NO:2016; and wherein the antibody inhibits TREM2 cleavage.

In some embodiments, the antibody is antibody clone 14D8, which is:
(1) an antibody, wherein the heavy chain variable region comprises the sequence of SEQ ID NO:1947 and the light chain variable region comprises the sequence of SEQ ID NO: 1957; and wherein the antibody inhibits TREM2 cleavage;
(2) an antibody, wherein the heavy chain variable region comprises a sequence having at least 85% identity to SEQ ID NO:1947, and the light chain variable region comprises a sequence having at least 85% identity to SEQ ID NO: 1957; and wherein the antibody inhibits TREM2 cleavage;
(3) an antibody, wherein the CDR1 of the heavy chain variable region comprises the amino acid sequence of SEQ ID NO:1967; the CDR2 of the heavy chain variable region comprises the amino acid sequence of SEQ ID NO: 1977; the CDR3 of the heavy chain variable region comprises the amino acid sequence of SEQ ID NO: 1987; the CDR1 of the light chain variable region comprises the amino acid sequence of SEQ ID NO: 1997; the CDR2 of the light chain variable region comprises the amino acid sequence of SEQ ID NO:2007; and the CDR3 of the light chain variable region comprises the amino acid sequence of SEQ ID NO:2017; and wherein the antibody inhibits TREM2 cleavage; or
(4) an antibody, wherein the CDR1 of the heavy chain variable region comprises an amino acid sequence having at least 70% identity to SEQ ID NO: 1967; the CDR2 of the heavy chain variable region comprises an amino acid sequence having at least 70% identity to SEQ ID NO: 1977; the CDR3 of the heavy chain variable region comprises an amino acid sequence having at least 70% identity to SEQ ID NO: 1987; the CDR1 of the light chain variable region comprises an amino acid sequence having at least 70% identity to SEQ ID NO: 1997; the CDR2 of the light chain variable region comprises an amino acid sequence having at least 60% identity to SEQ ID NO:2007; and the CDR3 of the light chain variable region comprises an amino acid sequence having at least 70% identity to SEQ ID NO:2017; and wherein the antibody inhibits TREM2 cleavage.

In some embodiments, the antibody is antibody clone 7A12, which is:
(1) an antibody, wherein the heavy chain variable region comprises the sequence of SEQ ID NO: 1948 and the light chain variable region comprises the sequence of SEQ ID NO: 1958; and wherein the antibody inhibits TREM2 cleavage;
(2) an antibody, wherein the heavy chain variable region comprises a sequence having at least 85%, preferably at least 90%, more preferably at least 95%, even more preferably at least 98%, and most preferably at least 99% identity to SEQ ID NO: 1948, and the light chain variable region comprises a sequence having at least 85%, preferably at least 90%, more preferably at least 95%, even more preferably at least 98%, and most preferably at least 99% identity to SEQ ID NO: 1958; and wherein the antibody inhibits TREM2 cleavage;
(3) an antibody, wherein the CDR1 of the heavy chain variable region comprises the amino acid sequence of SEQ ID NO: 1968; the CDR2 of the heavy chain variable region comprises the amino acid sequence of SEQ ID NO: 1978; the CDR3 of the heavy chain variable region comprises the amino acid sequence of SEQ ID NO: 1988; the CDR1 of the light chain variable region comprises the amino acid sequence of SEQ ID NO: 1998; the CDR2 of the light chain variable region comprises the amino acid sequence of SEQ ID NO:2008; and the CDR3 of the light chain variable region comprises the amino acid sequence of SEQ ID NO:2018; and wherein the antibody inhibits TREM2 cleavage; or
(4) an antibody, wherein the CDR1 of the heavy chain variable region comprises an amino acid sequence having at least 70%, preferably at least 75%, more preferably at least 80%, and most preferably at least 85% identity to SEQ ID NO: 1968; the CDR2 of the heavy chain variable region comprises an amino acid sequence having at least 70%, preferably at least 75%, more preferably at least 80%, even more preferably at least 85%, and most preferably at least 90% identity to SEQ ID NO:1978; the CDR3 of the heavy chain variable region comprises an amino acid sequence having at least 70%, preferably at least 75%, more preferably at least 80%, even more preferably at least 85%, and most preferably at least 90% identity to SEQ ID NO: 1988; the CDR1 of the light chain variable region comprises an amino acid sequence having at least 70%, preferably at least 75%, more preferably at least 80%, even more preferably at least 85%, and most preferably at least 90% identity to SEQ ID NO: 1998; the CDR2 of the light chain variable region comprises an amino acid sequence having at least 60%, preferably 100% identity to SEQ ID NO:2008; and the CDR3 of the light chain variable region comprises an amino acid sequence having at least 70%, preferably at least 75%, more preferably at least 80%, and most preferably at least 85% identity to SEQ ID NO:2018; and wherein the antibody inhibits TREM2 cleavage.

In some embodiments, the antibody is antibody clone 8A11, which is:
(1) an antibody, wherein the heavy chain variable region comprises the sequence of SEQ ID NO: 1949 and the light chain variable region comprises the sequence of SEQ ID NO: 1959; and wherein the antibody inhibits TREM2 cleavage;
(2) an antibody, wherein the heavy chain variable region comprises a sequence having at least 85%, preferably at least 90%, more preferably at least 95%, even more preferably at least 98%, and most preferably at least 99% identity to SEQ ID NO: 1949, and the light chain variable region comprises a sequence having at least 85%, preferably at least 90%, more preferably at least 95%, even more preferably at least 98%, and most preferably at least 99% identity to SEQ ID NO: 1959; and wherein the antibody inhibits TREM2 cleavage;
(3) an antibody, wherein the CDR1 of the heavy chain variable region comprises the amino acid sequence of SEQ ID NO: 1969; the CDR2 of the heavy chain variable region comprises the amino acid sequence of SEQ ID NO: 1979; the CDR3 of the heavy chain variable region comprises the amino acid sequence of SEQ ID NO: 1989; the CDR1 of the light chain variable region comprises the amino acid sequence of SEQ ID NO: 1999; the CDR2 of the light chain variable region comprises the amino acid sequence of SEQ ID NO:2009; and the CDR3 of the light chain variable region comprises the amino acid sequence of SEQ ID NO:2019; and wherein the antibody inhibits TREM2 cleavage; or
(4) an antibody, wherein the CDR1 of the heavy chain variable region comprises an amino acid sequence having at least 70%, preferably at least 75%, more preferably at least 80%, and most preferably at least 85% identity to SEQ ID NO: 1969; the CDR2 of the heavy chain variable region comprises an amino acid sequence having at least 70%, preferably at least 75%, more preferably at least 80%, even more preferably at least 85%, and most preferably at least 90% identity to SEQ ID NO:1979; the CDR3 of the heavy chain variable region comprises an amino acid sequence having at least 70%, preferably at least 75%, more preferably at least 80%, even more preferably at least 85%, and most preferably at least 90% identity to SEQ ID NO: 1989; the CDR1 of the light chain variable region comprises an amino acid sequence having at least 70%, preferably at least 75%, more preferably at least 80%, even more preferably at least 85%, and most preferably at least 90% identity to SEQ ID NO: 1999; the CDR2 of the light chain variable region comprises an amino acid sequence having at least 60%, preferably 100% identity to SEQ ID NO:2009; and the CDR3 of the light chain variable region comprises an amino acid sequence having at least 70%, preferably at least 75%, more preferably at least 80%, and most preferably at least 85% identity to SEQ ID NO:2019; and wherein the antibody inhibits TREM2 cleavage.

In some embodiments, the antibody is antibody clone 21A3, which is:
(1) an antibody, wherein the heavy chain variable region comprises the sequence of SEQ ID NO: 1950 and the light chain variable region comprises the sequence of SEQ ID NO: 1960; and wherein the antibody inhibits TREM2 cleavage;
(2) an antibody, wherein the heavy chain variable region comprises a sequence having at least 85%, preferably at least 90%, more preferably at least 95%, even more preferably at least 98%, and most preferably at least 99% identity to SEQ ID NO: 1950, and the light chain variable region comprises a sequence having at least 85%, preferably at least 90%, more preferably at least 95%, even more preferably at least 98%, and most preferably at least 99% identity to SEQ ID NO: 1960; and wherein the antibody inhibits TREM2 cleavage;
(3) an antibody, wherein the CDR1 of the heavy chain variable region comprises the amino acid sequence of SEQ ID NO: 1970; the CDR2 of the heavy chain variable region comprises the amino acid sequence of SEQ ID NO: 1980; the CDR3 of the heavy chain variable region comprises the amino acid sequence of SEQ ID NO: 1990; the CDR1 of the light chain variable region comprises the amino acid sequence of SEQ ID NO: 2000; the CDR2 of the light chain variable region comprises the amino acid sequence of SEQ ID NO:2010; and the CDR3 of the light chain variable region comprises the amino acid sequence of SEQ ID NO:2020; and wherein the antibody inhibits TREM2 cleavage; or
(4) an antibody, wherein the CDR1 of the heavy chain variable region comprises an amino acid sequence having at least 70%, preferably at least 75%, more preferably at least 80%, and most preferably at least 85% identity to SEQ ID NO: 1970; the CDR2 of the heavy chain variable region comprises an amino acid sequence having at least 70%, preferably at least 75%, more preferably at least 80%, even more preferably at least 85%, and most preferably at least 90% identity to SEQ ID NO:1980; the CDR3 of the heavy chain variable region comprises an amino acid sequence having at least 70%, preferably at least 75%, more preferably at least 80%, even more preferably at least 85%, and most preferably at least 90% identity to SEQ ID NO: 1990; the CDR1 of the light chain variable region comprises an amino acid sequence having at least 70%, preferably at least 75%, more preferably at least 80%, even more preferably at least 85%, and most preferably at least 90% identity to SEQ ID NO:2000; the CDR2 of the light chain variable region comprises an amino acid sequence having at least 60%, preferably 100% identity to SEQ ID NO:2010; and the CDR3 of the light chain variable region comprises an amino acid sequence having at least 70%, preferably at least 75%, more preferably at least 80%, and most preferably at least 85% identity to SEQ ID NO:2020; and wherein the antibody inhibits TREM2 cleavage.

In some embodiments, the antibody is antibody clone 10C3, which is:
(1) an antibody, wherein the heavy chain variable region comprises the sequence of SEQ ID NO: 1951 and the light chain variable region comprises the sequence of SEQ ID NO:1961; and wherein the antibody inhibits TREM2 cleavage;
(2) an antibody, wherein the heavy chain variable region comprises a sequence having at least 85%, preferably at least 90%, more preferably at least 95%, even more preferably at least 98%, and most preferably at least 99% identity to SEQ ID NO:1951, and the light chain variable region comprises a sequence having at least 85%, preferably at least 90%, more preferably at least 95%, even more preferably at least 98%, and most preferably at least 99% identity to SEQ ID NO:1961; and wherein the antibody inhibits TREM2 cleavage;
(3) an antibody, wherein the CDR1 of the heavy chain variable region comprises the amino acid sequence of SEQ ID NO: 1971; the CDR2 of the heavy chain variable region comprises the amino acid sequence of SEQ ID NO:1981; the CDR3 of the heavy chain variable region comprises the amino acid sequence of SEQ ID NO:1991; the CDR1 of the light chain variable region comprises the amino acid sequence of SEQ ID NO:2001; the CDR2 of the light chain variable region comprises the amino acid sequence of SEQ ID NO:2011; and the CDR3 of the light chain variable region comprises the amino acid sequence of SEQ ID NO:2021; and wherein the antibody inhibits TREM2 cleavage; or
(4) an antibody, wherein the CDR1 of the heavy chain variable region comprises an amino acid sequence having at least 70%, preferably at least 75%, more preferably at least 80%, and most preferably at least 85% identity to SEQ ID NO: 1971; the CDR2 of the heavy chain variable region comprises an amino acid sequence having at least 70%, preferably at least 75%, more preferably at least 80%, even more preferably at least 85%, and most preferably at least 90% identity to SEQ ID NO:1981; the CDR3 of the heavy chain variable region comprises an amino acid sequence having at least 70%, preferably at least 75%, more preferably at least 80%, even more preferably at least 85%, and most preferably at least 90% identity to SEQ ID NO:1991; the CDR1 of the light chain variable region comprises an amino acid sequence having at least 70%, preferably at least 75%, more preferably at least 80%, even more preferably at least 85%, and most preferably at least 90% identity to SEQ ID NO:2001; the CDR2 of the light chain variable region comprises an amino acid sequence having at least 60%, preferably 100% identity to SEQ ID NO:2011; and the CDR3 of the light chain variable region comprises an amino acid sequence having at least 70%, preferably at least 75%, more preferably at least 80%, and most preferably at least 85% identity to SEQ ID NO:2021; and wherein the antibody inhibits TREM2 cleavage.

In some embodiments, the antibody is antibody clone 18F9, which is:
(1) an antibody, wherein the heavy chain variable region comprises the sequence of SEQ ID NO: 1952 and the light chain variable region comprises the sequence of SEQ ID NO: 1962; and wherein the antibody inhibits TREM2 cleavage;
(2) an antibody, wherein the heavy chain variable region comprises a sequence having at least 85%, preferably at least 90%, more preferably at least 95%, even more preferably at least 98%, and most preferred at least 99% identity to SEQ ID NO: 1952, and the light chain variable region comprises a sequence having at least 85%, preferably at least 90%, more preferably at least 95%, even more preferably at least 98%, and most preferably at least 99% identity to SEQ ID NO: 1962; and wherein the antibody inhibits TREM2 cleavage;
(3) an antibody, wherein the CDR1 of the heavy chain variable region comprises the amino acid sequence of SEQ ID NO: 1972; the CDR2 of the heavy chain variable region comprises the amino acid sequence of SEQ ID NO: 1982; the CDR3 of the heavy chain variable region comprises the amino acid sequence of SEQ ID NO: 1992; the CDR1 of the light chain variable region comprises the amino acid sequence of SEQ ID NO: 2002; the CDR2 of the light chain variable region comprises the amino acid sequence of SEQ ID NO:2012; and the CDR3 of the light chain variable region comprises the amino acid sequence of SEQ ID NO:2022; and wherein the antibody inhibits TREM2 cleavage; or
(4) an antibody, wherein the CDR1 of the heavy chain variable region comprises an amino acid sequence having at least 70%, preferably at least 75%, more preferably at least 80%, and most preferably at least 85% identity to SEQ ID NO: 1972; the CDR2 of the heavy chain variable region comprises an amino acid sequence having at least 70%, preferably at least 75%, more preferably at least 80%, even more preferably at least 85%, and most preferably at least 90% identity to SEQ ID NO:1982; the CDR3 of the heavy chain variable region comprises an amino acid sequence having at least 70%, preferably at least 75%, more preferably at least 80%, even more preferably at least 85%, and most preferably at least 90% identity to SEQ ID NO: 1992; the CDR1 of the light chain variable region comprises an amino acid sequence having at least 70%, preferably at least 75%, more preferably at least 80%, even more preferably at least 85%, and most preferably at least 90% identity to SEQ ID NO:2002; the CDR2 of the light chain variable region comprises an amino acid sequence having at least 60%, preferably 100% identity to SEQ ID NO:2012; and the CDR3 of the light chain variable region comprises an amino acid sequence having at least 70%, preferably at least 75%, more preferably at least 80%, and most preferably at least 85% identity to SEQ ID NO:2022; and wherein the antibody inhibits TREM2 cleavage.

In some embodiments, the antibody is antibody clone 15C5, which is:
(1) an antibody, wherein the heavy chain variable region comprises the sequence of SEQ ID NO: 1953 and the light chain variable region comprises the sequence of SEQ ID NO: 1963; and wherein the antibody inhibits TREM2 cleavage;
(2) an antibody, wherein the heavy chain variable region comprises a sequence having at least 85%, preferably at least 90%, more preferably at least 95%, even more preferably at least 98%, and most preferably at least 99% identity to SEQ ID NO: 1953, and the light chain variable region comprises a sequence having at least 85%, preferably at least 90%, more preferably at least 95%, even more preferably at least 98%, and most preferably at least 99% identity to SEQ ID NO: 1963; and wherein the antibody inhibits TREM2 cleavage;
(3) an antibody, wherein the CDR1 of the heavy chain variable region comprises the amino acid sequence of SEQ ID NO: 1973; the CDR2 of the heavy chain variable region comprises the amino acid sequence of SEQ ID NO: 1983; the CDR3 of the heavy chain variable region comprises the amino acid sequence of SEQ ID NO: 1993; the CDR1 of the light chain variable region comprises the amino acid sequence of SEQ ID NO: 2003; the CDR2 of the light chain variable region comprises the amino acid sequence of SEQ ID NO:2013; and the CDR3 of the light chain variable region comprises the amino acid sequence of SEQ ID NO:2023; and wherein the antibody inhibits TREM2 cleavage; or
(4) an antibody, wherein the CDR1 of the heavy chain variable region comprises an amino acid sequence having at least 70%, preferably at least 75%, more preferably at least 80%, and most preferably at least 85% identity to SEQ ID NO: 1973; the CDR2 of the heavy chain variable region comprises an amino acid sequence having at least 70%, preferably at least 75%, more preferably at least 80%, even more preferably at least 85%, and most preferably at least 90% identity to SEQ ID NO:1983; the CDR3 of the heavy chain variable region comprises an amino acid sequence having at least 70%, preferably at least 75%, more preferably at least 80%, even more preferably at least 85%, and most preferably at least 90% identity to SEQ ID NO: 1993; the CDR1 of the light chain variable region comprises an amino acid sequence having at least 70%, preferably at least 75%, more preferably at least 80%, even more preferably at least 85%, and most preferably at least 90% identity to SEQ ID NO:2003; the CDR2 of the light chain variable region comprises an amino acid sequence having at least 60%, preferably 100% identity to SEQ ID NO:2013; and the CDR3 of the light chain variable region comprises an amino acid sequence having at least 70%, preferably at least 75%, more preferably at least 80%, and most preferably at least 85% identity to SEQ ID NO:2023; and wherein the antibody inhibits TREM2 cleavage.

In some embodiments, the antibody is antibody clone 1G6, which is:
(1) an antibody, wherein the heavy chain variable region comprises the sequence of SEQ ID NO: 1954 and the light chain variable region comprises the sequence of SEQ ID NO: 1964; and wherein the antibody inhibits TREM2 cleavage;
(2) an antibody, wherein the heavy chain variable region comprises a sequence having at least 85%, preferably at least 90%, more preferably at least 95%, even more preferably at least 98%, and most preferably at least 99% identity to SEQ ID NO: 1954, and the light chain variable region comprises a sequence having at least 85%, preferably at least 90%, more preferably at least 95%, even more preferably at least 98%, and most preferably at least 99% identity to SEQ ID NO: 1964; and wherein the antibody inhibits TREM2 cleavage;
(3) an antibody, wherein the CDR1 of the heavy chain variable region comprises the amino acid sequence of SEQ ID NO: 1974; the CDR2 of the heavy chain variable region comprises the amino acid sequence of SEQ ID NO: 1984; the CDR3 of the heavy chain variable region comprises the amino acid sequence of SEQ ID NO: 1994; the CDR1 of the light chain variable region comprises the amino acid sequence of SEQ ID NO: 2004; the CDR2 of the light chain variable region comprises the amino acid sequence of SEQ ID NO:2014; and the CDR3 of the light chain variable region comprises the amino acid sequence of SEQ ID NO:2024; and wherein the antibody inhibits TREM2 cleavage; or
(4) an antibody, wherein the CDR1 of the heavy chain variable region comprises an amino acid sequence having at least 70%, preferably at least 75%, more preferably at least 80%, and most preferably at least 85% identity to SEQ ID NO: 1974; the CDR2 of the heavy chain variable region comprises an amino acid sequence having at least 70%, preferably at least 75%, more preferably at least 80%, even more preferably at least 85%, and most preferably at least 90% identity to SEQ ID NO:1984; the CDR3 of the heavy chain variable region comprises an amino acid sequence having at least 70%, preferably at least 75%, more preferably at least 80%, even more preferably at least 85%, and most preferably at least 90% identity to SEQ ID NO: 1994; the CDR1 of the light chain variable region comprises an amino acid sequence having at least 70%, preferably at least 75%, more preferably at least 80%, even more preferably at least 85%, and most preferably at least 90% identity to SEQ ID NO:2004; the CDR2 of the light chain variable region comprises an amino acid sequence having at least 60%, preferably 100% identity to SEQ ID NO:2014; and the CDR3 of the light chain variable region comprises an amino acid sequence having at least 70%, preferably at least 75%, more preferably at least 80%, and most preferably at least 85% identity to SEQ ID NO:2024; and wherein the antibody inhibits TREM2 cleavage.

In some embodiments, the antibody is an antibody disclosed in FIG. 9 of PCT Patent Application Publication No. WO2018/015573A1, reproduced below as TABLES F1-F4.

TABLE F1

| Clone | |
|---|---|
| name | Variable region of the heavy chain |
| 14D3 | EVKLLEFGGGLVQPGGSMRLSCAASGFTFTDFYMNW |
| | IRQPAGRAPEWLGLIRNKTKGYTTEYNRSVKGRFTI |
| | SRDNTQNMLYLQMNSLRPEDTATYYCARIGVNNGGS |
| | LDYWGQGVMVTVSS (SEQ ID NO: 1946) |
| 14D8 | EVKLLESGGGLVQPGGSMRLSCAASGFTFTDFYMNW |
| | IRQPAGKAPEWLGLIRNKANGYTTVYNPSVKGRFTI |
| | SRDNTQNMLYLQMNTLRGEDTATYYCARIGINNGGS |
| | LDYWGQGVMVTVSS (SEQ ID NO: 1947) |
| 7A12 | EVKLLESGGGLVQPGGSMRLSCAASGFTFTDFYMNW |
| | IRQPAGKAPEWLGLIRNKANGYTTQYNPSVKGRFTI |
| | SRDNTQNMLYLQMNTLRGEDTATYYCARIGINNGGS |
| | LDYWGQGVMVTVSS (SEQ ID NO: 1948) |
| 8A11 | EVKLLESGGGLVQPGGSMRLSCAASGFTFTDFYMNW |
| | IRQPAGKAPEWLGLIRNKTKGYTTEYNTSVKGRFTI |
| | SRDNTQNMLYLQMNSLRPEDTATYYCARIGVNNGGS |
| | LDYWGQGVMVTVSS (SEQ ID NO: 1949) |
| 21A3 | EVKLLESGGGLVQPGGSMRLSCAASGFTFTDFYMNW |
| | IRQPAGKAPEWLGLIRNKANGYTTQYNPSVKGRFTI |
| | SRDNTQNMLYLQMNTLRGEDTATYYCARIGINNGGS |
| | LDYWGQGVMVTVSS (SEQ ID NO: 1950) |
| 10C3 | EVKLLESGGGLVQPGGSMRLSCAASGFTFTDFYMNW |
| | IRQPAGETPEWLGLIRNKTKGYTTEYNPSVKGRFTI |
| | SRDNTQNMLYLQMNSLRPEDTATYYCARIGTNNGGS |
| | LDYWGQGVMVTVSS (SEQ ID NO: 1951) |
| 18F9 | EVKLLESGGGLVQPGGSMRLSCVVSGFTFTDFYMNW |
| | IRQAAGKAPEWLGLIRNKVNGYRTEYNPSVKGRFTI |
| | SRDNIQNMLYLQMNTLRAEDTATYYCARIGINNGGS |
| | LDYWGQGVMVTVSS (SEQ ID NO: 1952) |
| 15C5 | EVKLLESGGGLVQPGGSMRLSCAASGFTFTDFYMNW |
| | IRQPAGKAPEWLGLIRNKAYGYTTEYNPSVKGRFTI |
| | SRDNTQDMLYLQMNTLRAEDTATYYCARIGINYGGS |
| | LDYWGQGVMVTVSS (SEQ ID NO: 1953) |
| 1G6 | EVKLLESGGGLVQPGGSLRLSCVASGFTFTDFYMNW |
| | IRQPAGKAPEWLGLIRNKANGFTTEYNPSVKGRFTI |
| | SRDNTQHMLYLQMNTLRAEDTATYYCARIGINNGGS |
| | LDYWGQGVMVTVSS (SEQ ID NO: 1954) |
| Consensus | EVKLLESGGGLVQPGGSMRLSCAASGFTFTDFYMNW |
| sequence | IRQPAGKAPEWLGLIRNKANGYTTEYNPSVKGRFTI |
| | SRDNTQNMLYLQMNTLREDTATYYCARIGINNGGSL |
| | DYWGQGVMVTVSS (SEQ ID NO: 1955) |

TABLE F2

| Clone | |
|---|---|
| name | Variable region of the light chain |
| 14D3 | DILIIQSPASLTVSAGARVTMSCKSSQSLLYSENNQ |
| | DYLAWYQQKPGQFPKLLIYGASNRHTGVPDRFTGSG |
| | SGTDFTLTISSVQAEDLADYYCEQTYSYPYTFGAGT |
| | KLELK (SEQ ID NO: 1956) |
| 14D8 | DILINQSPASLTVSTGEKVTMSCRSSQSLLYSEKNQ |
| | DYLAWYQQKPGQFPKLLIYGASYRHTGVPDRFTGSG |
| | SGTDFTLTISSVQAEDLADYYCEQTYSYPYTFGAGT |
| | KLELK (SEQ ID NO: 1957) |
| 7A12 | DILINQSPASLTVSAGEKVTMSCKSSQSLLYSEKNQ |
| | DYLAWYQQKPGQSPKLLMYGASYRHTGVPDRFTGSG |
| | SGTDFTLTISSVQAEDLADYYCEQTYSYPYTFGAGT |
| | KLELK (SEQ ID NO: 1958) |
| 8A11 | DILIIQSPASLTVSAGARVTMSCKSSQSLLYSENNQ |
| | DYLAWYQQKPGQFPKLLIYGASNRHTGVPDRFTGSG |
| | SGTDFTLTISSVQAEDLADYYCEQTYSYPYTFGAGT |
| | KLELK (SEQ ID NO: 1959) |
| 21A3 | DILINQSPASLTVSAGEKVTMSCKSSQSLLYSEKNQ |
| | DYLAWYQQKPGQSPKLLMYGASYRHTGVPDRFTGSG |
| | SGTDFTLTISSVQAEDLADYYCEQTYSYPYTFGAGT |
| | KLELK (SEQ ID NO: 1960) |
| 10C3 | DILIIQSPASLTVSAGARVTMSCKSSQSLLYSENNQ |
| | DYLAWYQQKPGQFPKLLIYGASNRHTGVPDRFTGSG |
| | SGTDFTLTISSVQAEDLADYYCEQTYSYPYTFGAGT |
| | KLELK (SEQ ID NO: 1961) |
| 18F9 | DILINQSPASLTVSAGEKVTMSCKSSQSLLYSENNQ |
| | DYLAWYQQKPGQFPKLLIYGASNRHTGVPDRFTGSG |
| | SGTDFTLTISSVQAEDLADYYCEQTYSYPYTFGAG |
| | TKLELK (SEQ ID NO: 1962) |
| 15C5 | DILINQSPASLTVSAGEKVTVSCKSSQSLLYSESNQ |
| | DYLAWYQQKPGQFPKLLIYGASYRHTGVPDRFTGSG |
| | SGTDFTLTISSVQAEDLAHYYCEQTYSYPYTFGAGT |
| | KLELK (SEQ ID NO: 1963) |
| 1G6 | DILINQSPASLTVSTGEKVTMSCKSSQSLLYSENKQ |
| | DYLAWYQQKPGQFPKLLIYGASNRHTGVPDRFTGSG |
| | SGTDFTLTINIVQAEDLADYYCEQTYSYPYTFGAGT |
| | KLELK (SEQ ID NO: 1964) |
| Consensus | DILINQSPASLTVSAGEKVTMSCKSSQSLLYSENNQ |
| sequence | DYLAWYQQKPGQFPKLLIYGASNRHTGVPDRFTGSG |
| | SGTDFTLTISSVQAEDLADYYCEQTYSYPYTFGAGT |
| | KLELK (SEQ ID NO: 1965) |

TABLE F3

| | Complementarity determining regions in | | |
|---|---|---|---|
| Clone | the variable region of the heavy chain | | |
| name | CDR1 | CDR2 | CDR3 |
| 14D3 | GFTFTDFY | IRNKTKGYTT | ARIGVNNGGSLDYWG |
| | (SEQ ID | (SEQ ID | (SEQ ID NO: 1986) |
| | NO: 1966) | NO: 1976) | |
| 14D8 | GFTFTDFY | IRNKANGYTT | ARIGINNGGSLDYWG |
| | (SEQ ID | (SEQ ID | (SEQ ID NO: 1987) |
| | NO: 1967) | NO: 1977) | |
| 7Al2 | GFTFTDFY | IRNKANGYTT | ARIGINNGGSLDYWG |
| | (SEQ ID | (SEQ ID | (SEQ ID NO: 1988) |
| | NO: 1968) | NO: 1978) | |
| 8A11 | GFTFTDFY | IRNKTKGYTT | ARIGVNNGGSLDYWG |
| | (SEQ ID | (SEQ ID | (SEQ ID NO: 1989) |
| | NO: 1969) | NO: 1979) | |
| 21A3 | GFTFTDFY | IRNKANGYTT | ARIGINNGGSLDYWG |
| | (SEQ ID | (SEQ ID | (SEQ ID NO: 1990) |
| | NO: 1970) | NO: 1980) | |
| 10C3 | GFTFTDFY | IRNKTKGYTT | ARIGTNNGGSLDYWG |
| | (SEQ ID | (SEQ ID | (SEQ ID NO: 1991) |
| | NO: 1971) | NO: 1981) | |
| 18F9 | GFTFTDFY | IRNKVNGYRT | ARIGINNGGSLDYWG |
| | (SEQ ID | (SEQ ID | (SEQ ID NO: 1992) |
| | NO: 1972) | NO: 1982) | |
| 15C5 | GFTFTDFY | IRNKAYGYTT | ARIGINYGGSLDYWG |
| | (SEQ ID | (SEQ ID | (SEQ ID NO: 1993) |
| | NO: 1973) | NO: 1983) | |
| 1G6 | GFTFTDFY | IRNKANGFTT | ARIGINNGGSLDYWG |
| | (SEQ ID | (SEQ ID | (SEQ ID NO: 1994) |
| | NO: 1974) | NO: 1984) | |
| Consensus | GFTFTDFY | IRNKANGYTT | ARIGINNGGSLDYWG |
| | (SEQ ID | (SEQ ID | (SEQ ID NO: 1995) |
| | NO: 1975) | NO: 1985) | |

TABLE F4

| | Complementarity determining regions in | | |
|---|---|---|---|
| Clone | the variable region of the light chain | | |
| name | CDR1 | CDR2 | CDR3 |
| 14D3 | QSLLYSENNQDY | GAS | EQTYSYPYT |
| | (SEQ ID NO: 1996) | (SEQ ID | (SEQ ID |
| | | NO: 2006) | NO: 2016) |
| 14D8 | QSLLYSEKNQDY | GAS | EQTYSYPYT |
| | (SEQ ID NO: 1997) | (SEQ ID | (SEQ ID |
| | | NO: 2007) | NO: 2017) |
| 7Al2 | QSLLYSEKNQDY | GAS | EQTYSYPYT |
| | (SEQ ID NO: 1998) | (SEQ ID | (SEQ ID |
| | | NO: 2008) | NO: 2018) |
| 8A11 | QSLLYSENNQDY | GAS | EQTYSYPYT |
| | (SEQ ID NO: 1999) | (SEQ ID | (SEQ ID |
| | | NO: 2009) | NO: 2019) |
| 21A3 | QSLLYSEKNQDY | GAS | EQTYSYPYT |
| | (SEQ ID NO: 2000) | (SEQ ID | (SEQ ID |
| | | NO: 2010) | NO: 2020) |
| 10C3 | QSLLYSENNQDY | GAS | EQTYSYPYT |
| | (SEQ ID NO: 2001) | (SEQ ID | (SEQ ID |
| | | NO: 2011) | NO: 2021) |
| 18F9 | QSLLYSENNQDY | GAS | EQTYSYPYT |
| | (SEQ ID NO: 2002) | (SEQ ID | (SEQ ID |
| | | NO: 2012) | NO: 2022) |
| 15C5 | QSLLYSESNQDY | GAS | EQTYSYPYT |
| | (SEQ ID NO: 2003) | (SEQ ID | (SEQ ID |
| | | NO: 2013) | NO: 2023) |
| 1G6 | QSLLYSENKQDY | GAS | EQTYSYPYT |
| | (SEQ ID NO: 2004) | (SEQ ID | (SEQ ID |
| | | NO: 2014) | NO: 2024) |
| Consensus | QSLLYSENNQDY | GAS | EQTYSYPYT |
| | (SEQ ID NO: 2005) | (SEQ ID | (SEQ ID |
| | | NO: 2015) | NO: 2025) |

In some embodiments, each of the light chain variable regions and each of the heavy chain variable regions disclosed in in the above tables as well as specific combinations thereof and other embodiments of the anti-TREM2 antibody described in the '573 application and herein may be attached to the light chain constant regions (TABLE EN1) and heavy chain constant regions (TABLE EN2) to form complete antibody light and heavy chains, respectively, as further discussed below. Further, each of the generated heavy and light chain sequences may be combined to form a complete antibody structure. It should be understood that the heavy chain and light chain variable regions provided herein can also be attached to other constant domains having different sequences than the exemplary sequences listed herein.

G. PCT Patent Application Publication No. WO2019/055841A1

In some embodiments, the TREM2 agonist is an antibody or an antigen-binding fragment thereof, as described in PCT Patent Application Publication No. WO2019/055841A1 (“the '841 application”), which is incorporated by reference herein, in its entirety.

In some embodiments, the TREM2 binding agent comprises an antibody that comprises a light chain variable domain comprising a CDRL1, CDRL2, and CDRL3, and a heavy chain variable domain comprising a CDRH1, CDRH2, and CDRH3 disclosed in the '841 application specification. In some embodiments, the TREM2 binding agent comprises an antibody that comprises a light chain variable domain and a heavy chain variable domain disclosed in the '841 application specification.

In some embodiments, the antibody comprises one or more (e.g., one, two, three, four, five, or all six) CDRs selected from the group consisting of:
(a) a heavy chain CDR1 sequence having at least 90% sequence identity to the amino acid sequence of any one of SEQ ID NOS:2049, 2077, 2080, 2086, 2092, 2098, 2103, 2109, 2115, 2122, 2126, 2347, and 2355 or having up to two amino acid substitutions relative to the amino acid sequence of any one of SEQ ID NOS:2049, 2077, 2080, 2086, 2092, 2098, 2103, 2109, 2115, 2122, 2126, 2347, and 2355;
(b) a heavy chain CDR2 sequence having at least 90% sequence identity to the amino acid sequence of any one of SEQ ID NOS:2050, 2078, 2081, 2087, 2093, 2099, 2104, 2110, 2116, 2120, 2123, 2127, 2348, and 2356 or having up to two amino acid substitutions relative to the amino acid sequence of any one of SEQ ID NOS:2050, 2078, 2081, 2087, 2093, 2099, 2104, 2110, 2116, 2120, 2123, 2127, 2348, and 2356;
(c) a heavy chain CDR3 sequence having at least 90% sequence identity to the amino acid sequence of any one of SEQ ID NOS:2051, 2082, 2088, 2094, 2100, 2105, 2111, 2117, 2124, 2128, 2349, and 2357 or having up to two amino acid substitutions relative to the amino acid sequence of any one of SEQ ID NOS:2051, 2082, 2088, 2094, 2100, 2105, 2111, 2117, 2124, 2128, 2349, and 2357;
(d) a light chain CDR1 sequence having at least 90% sequence identity to the amino acid sequence of any one of SEQ ID NOS:2052, 2083, 2089, 2095, 2101, 2106, 2112, 2118, 2129, and 2351 or having up to two amino acid substitutions relative to the amino acid sequence of any one of SEQ ID NOS:2052, 2083, 2089, 2095, 2101, 2106, 2112, 2118, 2129, and 2351;
(e) a light chain CDR2 sequence having at least 90% sequence identity to the amino acid sequence of any one of SEQ ID NOS:2053, 2079, 2084, 2090, 2096, 2107, 2113, 2352, and 2359 or having up to two amino acid substitutions relative to the amino acid sequence of any one of SEQ ID NOS:2053, 2079, 2084, 2090, 2096, 2107, 2113, 2352, and 2359; and
(f) a light chain CDR3 sequence having at least 90% sequence identity to the amino acid sequence of any one of SEQ ID NOS:2054, 2085, 2091, 2097, 2102, 2108, 2114, 2119, 2121, 2125, 2130, and 2353 or having up to two amino acid substitutions relative to the amino acid sequence of any one of SEQ ID NOS:2054, 2085, 2091, 2097, 2102, 2108, 2114, 2119, 2121, 2125, 2130, and 2353.

In some embodiments, the antibody comprises:
(a) a heavy chain CDR1 sequence comprising the amino acid sequence of SEQ ID NO:2049, a heavy chain CDR2 sequence comprising the amino acid sequence of SEQ ID NO:2050, a heavy chain CDR3 sequence comprising the amino acid sequence of SEQ ID NO:2051, a light chain CDR1 sequence comprising the amino acid sequence of SEQ ID NO:2052, a light chain CDR2 sequence comprising the amino acid sequence of SEQ ID NO:2052, and a light chain CDR3 sequence comprising the amino acid sequence of SEQ ID NO:2053; or
(b) a heavy chain CDR1 sequence comprising the amino acid sequence of SEQ ID NO:2077, a heavy chain CDR2 sequence comprising the amino acid sequence of SEQ ID NO:2078, a heavy chain CDR3 sequence comprising the amino acid sequence of SEQ ID NO:2051, a light chain CDR1 sequence comprising the amino acid sequence of SEQ ID NO:2052, a light chain CDR2 sequence comprising the amino acid sequence of SEQ ID NO:2079, and a light chain CDR3 sequence comprising the amino acid sequence of SEQ ID NO:2054; or
(c) a heavy chain CDR1 sequence comprising the amino acid sequence of SEQ ID NO:2080, a heavy chain CDR2 sequence comprising the amino acid sequence of SEQ ID NO:2081, a heavy chain CDR3 sequence comprising the amino acid sequence of SEQ ID NO:2082, a light chain CDR1 sequence comprising the amino acid sequence of SEQ ID NO:2083, a light chain CDR2 sequence comprising the amino acid sequence of SEQ ID NO:2084, and a light chain CDR3 sequence comprising the amino acid sequence of SEQ ID NO:2085; or
(d) a heavy chain CDR1 sequence comprising the amino acid sequence of SEQ ID NO:2086, a heavy chain CDR2 sequence comprising the amino acid sequence of SEQ ID NO:2087, a heavy chain CDR3 sequence comprising the amino acid sequence of SEQ ID NO:2088, a light chain CDR1 sequence comprising the amino acid sequence of SEQ ID NO:2089, a light chain CDR2 sequence comprising the amino acid sequence of SEQ ID NO:2090, and a light chain CDR3 sequence comprising the amino acid sequence of SEQ ID NO:2091; or
(e) a heavy chain CDR1 sequence comprising the amino acid sequence of SEQ ID NO:2092, a heavy chain CDR2 sequence comprising the amino acid sequence of SEQ ID NO:2093, a heavy chain CDR3 sequence comprising the amino acid sequence of SEQ ID NO:2094, a light chain CDR1 sequence comprising the amino acid sequence of SEQ ID NO:2095, a light chain CDR2 sequence comprising the amino acid sequence of SEQ ID NO:2096, and a light chain CDR3 sequence comprising the amino acid sequence of SEQ ID NO:2097; or (f) a heavy chain CDR1 sequence comprising the amino acid sequence of SEQ ID NO:2098, a heavy chain CDR2 sequence comprising the amino acid sequence of SEQ ID NO:2099, a heavy chain CDR3 sequence comprising the amino acid sequence of SEQ ID NO:2100, a light chain CDR1 sequence comprising the amino acid sequence of SEQ ID NO:2101, a light chain CDR2 sequence comprising the amino acid sequence of SEQ ID NO:2079, and a light chain CDR3 sequence comprising the amino acid sequence of SEQ ID NO:2102; or
(g) a heavy chain CDR1 sequence comprising the amino acid sequence of SEQ ID NO:2103, a heavy chain CDR2 sequence comprising the amino acid sequence of SEQ ID NO:2104, a heavy chain CDR3 sequence comprising the amino acid sequence of SEQ ID NO:2105, a light chain CDR1 sequence comprising the amino acid sequence of SEQ ID NO:2106, a light chain CDR2 sequence comprising the amino acid sequence of SEQ ID NO:2107, and a light chain CDR3 sequence comprising the amino acid sequence of SEQ ID NO:2108; or
(h) a heavy chain CDR1 sequence comprising the amino acid sequence of SEQ ID NO:2109, a heavy chain CDR2 sequence comprising the amino acid sequence of SEQ ID NO:2110, a heavy chain CDR3 sequence comprising the amino acid sequence of SEQ ID NO:2111, a light chain CDR1 sequence comprising the amino acid sequence of SEQ ID NO:2112, a light chain CDR2 sequence comprising the amino acid sequence of SEQ ID NO:2113, and a light chain CDR3 sequence comprising the amino acid sequence of SEQ ID NO:2114; or
(i) a heavy chain CDR1 sequence comprising the amino acid sequence of SEQ ID NO:2115, a heavy chain CDR2 sequence comprising the amino acid sequence of SEQ ID NO:2116, a heavy chain CDR3 sequence comprising the amino acid sequence of SEQ ID NO:2117, a light chain CDR1 sequence comprising the amino acid sequence of SEQ ID NO:2118, a light chain CDR2 sequence comprising the amino acid sequence of SEQ ID NO:2119, and a light chain CDR3 sequence comprising the amino acid sequence of SEQ ID NO:2119; or
(j) a heavy chain CDR1 sequence comprising the amino acid sequence of SEQ ID NO:2115, a heavy chain CDR2 sequence comprising the amino acid sequence of SEQ ID NO:2120, a heavy chain CDR3 sequence comprising the amino acid sequence of SEQ ID NO:2117, a light chain CDR1 sequence comprising the amino acid sequence of SEQ ID NO:2118, a light chain CDR2 sequence comprising the amino acid sequence of SEQ ID NO:2079, and a light chain CDR3 sequence comprising the amino acid sequence of SEQ ID NO:2121; or
(k) a heavy chain CDR1 sequence comprising the amino acid sequence of SEQ ID NO:2123, a heavy chain CDR2 sequence comprising the amino acid sequence of SEQ ID NO:2132, a heavy chain CDR3 sequence comprising the amino acid sequence of SEQ ID NO:2133, a light chain CDR1 sequence comprising the amino acid sequence of SEQ ID NO:2102, a light chain CDR2 sequence comprising the amino acid sequence of SEQ ID NO:2079, and a light chain CDR3 sequence comprising the amino acid sequence of SEQ ID NO:2125; or
(l) a heavy chain CDR1 sequence comprising the amino acid sequence of SEQ ID NO:2126, a heavy chain CDR2 sequence comprising the amino acid sequence of SEQ ID NO:2127, a heavy chain CDR3 sequence comprising the amino acid sequence of SEQ ID NO:2128, a light chain CDR1 sequence comprising the amino acid sequence of SEQ ID NO:2129, a light chain CDR2 sequence comprising the amino acid sequence of SEQ ID NO:2079, and a light chain CDR3 sequence comprising the amino acid sequence of SEQ ID NO:2130; or
(m) a heavy chain CDR1 sequence comprising the amino acid sequence of SEQ ID NO:2347, a heavy chain CDR2 sequence comprising the amino acid sequence of SEQ ID NO:2348, a heavy chain CDR3 sequence comprising the amino acid sequence of SEQ ID NO:2349, a light chain CDR1 sequence comprising the amino acid sequence of SEQ ID NO:2351, a light chain CDR2 sequence comprising the amino acid sequence of SEQ ID NO:2352, and a light chain CDR3 sequence comprising the amino acid sequence of SEQ ID NO:2353; or
(n) a heavy chain CDR1 sequence comprising the amino acid sequence of SEQ ID NO:2355, a heavy chain CDR2 sequence comprising the amino acid sequence of SEQ ID NO:2356, a heavy chain CDR3 sequence comprising the amino acid sequence of SEQ ID NO:2357, a light chain CDR1 sequence comprising the amino acid sequence of SEQ ID NO:2089, a light chain CDR2 sequence comprising the amino acid sequence of SEQ ID NO:2359, and a light chain CDR3 sequence comprising the amino acid sequence of SEQ ID NO:2091.

In some embodiments, the antibody or antigen-binding portion thereof comprises:
(a) a heavy chain variable region comprising an amino acid sequence that has at least 90% sequence identity to SEQ ID NO:2047; and a light chain variable region comprising an amino acid sequence that has at least 90% sequence identity to SEQ ID NO:2048; or
(b) a heavy chain variable region comprising an amino acid sequence that has at least 90% sequence identity to SEQ ID NO:2055; and a light chain variable region comprising an amino acid sequence that has at least 90% sequence identity to SEQ ID NO:2066; or
(c) a heavy chain variable region comprising an amino acid sequence that has at least 90% sequence identity to SEQ ID NO:2056; and a light chain variable region comprising an amino acid sequence that has at least 90% sequence identity to SEQ ID NO:2067; or
(d) a heavy chain variable region comprising an amino acid sequence that has at least 90% sequence identity to SEQ ID NO:2057; and a light chain variable region comprising an amino acid sequence that has at least 90% sequence identity to SEQ ID NO:2068; or
(e) a heavy chain variable region comprising an amino acid sequence that has at least 90% sequence identity to SEQ ID NO:2058; and a light chain variable region comprising an amino acid sequence that has at least 90% sequence identity to SEQ ID NO:2069; or
(f) a heavy chain variable region comprising an amino acid sequence that has at least 90% sequence identity to SEQ ID NO:2059; and a light chain variable region comprising an amino acid sequence that has at least 90% sequence identity to SEQ ID NO:2070; or
(g) a heavy chain variable region comprising an amino acid sequence that has at least 90% sequence identity to SEQ ID NO:2060; and a light chain variable region comprising an amino acid sequence that has at least 90% sequence identity to SEQ ID NO:2071; or
(h) a heavy chain variable region comprising an amino acid sequence that has at least 90% sequence identity to SEQ ID NO:2061; and a light chain variable region comprising an amino acid sequence that has at least 90% sequence identity to SEQ ID NO:2072; or
(i) a heavy chain variable region comprising an amino acid sequence that has at least 90% sequence identity to SEQ ID NO:2062; and a light chain variable region comprising an amino acid sequence that has at least 90% sequence identity to SEQ ID NO:2073; or
(j) a heavy chain variable region comprising an amino acid sequence that has at least 90% sequence identity to SEQ ID NO:2063; and a light chain variable region comprising an amino acid sequence that has at least 90% sequence identity to SEQ ID NO:2074; or
(k) a heavy chain variable region comprising an amino acid sequence that has at least 90% sequence identity to SEQ ID NO:2064; and a light chain variable region comprising an amino acid sequence that has at least 90% sequence identity to SEQ ID NO:2075; or
(l) a heavy chain variable region comprising an amino acid sequence that has at least 90% sequence identity to SEQ ID NO:2065; and a light chain variable region comprising an amino acid sequence that has at least 90% sequence identity to SEQ ID NO:2076; or
(m) a heavy chain variable region comprising an amino acid sequence that has at least 90% sequence identity to SEQ ID NO:2346, and a light chain variable region comprising an amino acid sequence that has at least 90% sequence identity to SEQ ID NO:2350; or
(n) a heavy chain variable region comprising an amino acid sequence that has at least 90% sequence identity to SEQ ID NO:2354, and a light chain variable region comprising an amino acid sequence that has at least 90% sequence identity to SEQ ID NO:2358.

In some embodiments, the antibody is an antibody disclosed in Table 15 ofPCT Patent Application Publication No. WO2019/05584A1, reproduced as TABLE G1 below. In some embodiments, the antibody is an antibody comprises a light chain variable domain comprising a CDRL1, CDRL2, and CDRL3, and a heavy chain variable domain comprising a CDRH1, CDRH2, and CDRH3 disclosed in TABLE G1.

TABLE G1

| Description | Sequence |
|---|---|
| muIgG1 3′ primer | GGACAGGGATCCAGAGTTCC (SEQ ID NO: 2042) |
| muIgG2 3′ primer | AGCTGGGAAGGTGTGCACAC (SEQ ID NO: 2043) |
| muIgG3 3′ primer | CAGGGGCCAGTGGATAGAC (SEQ ID NO: 2044) |
| muCkappa.1 3′ | GACATTGATGTCTTTGGGGT (SEQ ID NO: 2045) |
| primer | |
| muCkappa.2 3′ | TTCACTGCCATCAATCTTCC (SEQ ID NO: 2046) |
| primer | |
| R59.F6 VH amino | QVQLQQPGAELVKPGASVKLSCKASGYTFTSYWMHWVKQSPGRGLE |
| acid sequence | WIGRSDPTTGGTNYNEKFKTKATLTVDKPSSTAYMQLSSLTSDDSAV |
| | YYCVRTSGTGDYWGQGTSLTVSSAKTTAPSVYPLAPVCGGTTGSSVT |
| | (SEQ ID NO: 2047) |
| R59.F6 VL amino | DVVMTQTPLSLPVSLGDQASISCRSSQSLVHNNGNTFLHWYLQKPGQ |
| acid sequence | SPKWYkVSNRFSGVPDRFSGSGSGTDFTLKISRVEAEDLGVYFCSQTT |
| | HVPPTFGGGTKLEIKRADAAPTVSIFPPSSEQLTSGGASVVCF |
| | (SEQ ID NO: 2048) |
| R59.F6 CDR-H1 | GYTFTSY (SEQ ID NO: 2049) |
| amino acid sequence | |
| R59.F6 CDR-H2 | IGRSDPTTGGTNYNE (SEQ ID NO: 2050) |
| amino acid sequence | |
| R59.F6 and RS.F10 | VRTSGTGDY (SEQ ID NO: 2051) |
| CDR-H3 amino acid | |
| sequence | |
| R59.F6 and RS.F10 | RSSQSLVHNNGNTFLH (SEQ ID NO: 2052) |
| CDR-L1 amino acid | |
| sequence | |
| R59.F6 CDR-L2 | VSNRFS (SEQ ID NO: 2053) |
| amino acid sequence | |
| R59.F6 and RS.F10 | SQTTHVPPT (SEQ ID NO: 2054) |
| CDR-L3 amino acid | |
| sequence | |
| 21D11 VH amino | QVQLQQSGAELARPGASVKLSCKASGYTFTSYWIQWVKQRPGQGLE |
| acid sequence | WIGTIYPGDGDARYTQKFKGKATLTADKSSSTTYMQLNSLASEDSAV |
| | YYCARNGITTAGYYAMDYWGQGTSVTVSS (SEQ ID NO: 2055) |
| 21D4.D1 VH amino | QVQLQQSGADLLRPGVSVKISCKGSGYTFTDHAMHWVKQSHAESLE |
| acid sequence | WIGVISTYSGDTGYNQKFKGKATMTVDKSSSTAYLELARLTSEDSAIY |
| | YCAREGHYDDAMDYWGQGTSVTVSS (SEQ ID NO: 2056) |
| 26D2 VH amino acid | EVQLQQSGPELVKPGASVKMSCKASGYTFTSYVMHWVKQKPGQGLE |
| sequence | WIGYINPYTDGTKYNEKFKGKATLTSDKSSSTAYMDLSSLTSEDSAVY |
| | YCARGEVRRYALDYWGQGTSVTVSS (SEQ ID NO: 2057) |
| 26E2.A3 VH amino | QVHLQQSGSELRSPGSSVKLSCKDFDSEVFPISYMSWIRQKPGHGFEW |
| acid sequence; | IGDILPSIGGRIYGVKFEDRATLDADTVSNTAYLELNSLTSEDSAIYYC |
| 24B4.A1 VH amino | ARKDYGSLAYWGQGTLVTVSA (SEQ ID NO: 2058) |
| acid sequence | |
| 3D3.A1 VH amino | EVQLQQSGPELVKPGASVKISCKTSGYTLSEYTMHWVIQSHGKSLEWI |
| acid sequence | GGVIPNSGGTSYNQKFRDKASLTVDKSSSTAYLELRSLTSEDSAVYYC |
| | ARGDDSYRRGYALDYWGQGTSVTVSS (SEQ ID NO: 2059) |
| 40H3.A4 VH amino | EVQLQQSGAEVVKPGASVKLSCTASGFNIKDTYMHWVKQRPEQGLE |
| acid sequence | WIGRIDPANGNTKYDPKFQGKATITADTSSNTAYLQLSSLTSEDTAVY |
| | YCATLFAYWGQGTLVTVSA (SEQ ID NO: 2060) |
| 42E8.H1 VH amino | DVQLQESGPGLVKPSQSLSLTCTVTGYSITSDYAWNWIRQFPGNKLE |
| acid sequence | WMGYINYSGRTIYNPSLKSRISITRDTSKNHFFLQLISVTTEDTATYYC |
| | ARWNGNYGFAYWGQGTLVTVSA (SEQ ID NO: 2061) |
| 49H1 LB1 VH amino | DVQLQESGPGLVKPSQSLSLTCTVTGYSITSDYAWNWIRQFPGNRLE |
| acid sequence | WMGYISFSGSTSYNPSLKSRISITRDTSKNQFFLQLNSVTTEDTATYYC |
| | ARWNGNYGFAYWGQGTLVTVSA (SEQ ID NO: 2062) |
| 54C2.A1 VH amino | QVHLQQSGSELRSPGSSVKLSCKDFDSEVFPIAYMSWVRQKPGHGFE |
| acid sequence | WIGDILPSIGRRIYGVKFEDKATLDADTVSNTAYLELNSLTSEDSAIYY |
| | CTRKDYGSLAYWGQGTLVTVSA (SEQ ID NO: 2063) |
| 57D7.A1 VH amino | QVQLKESGPGLVAPSQSLSITCTVSGFSLSRYSVYWVRQPPGKGLEWL |
| acid sequence | GMIWGGGNTDYNSALKSRLSISKDNSKSQVFLKMNSLQTDDSAMYY |
| | CVQYGGMDYWGQGTSVTVSS (SEQ ID NO: 2064) |
| RS9.F6 VH amino | QVQLQQPGAELVKPGASVKLSCKASGYTFTSYWMHWVKQSPGRGLE |
| acid sequence; | WIGRSDPTTGGTNYNEKFKTKATLTVDKPSSTAYMQLSSLTSDDSAV |
| RS.F10 VH amino | YYCVRTSGTGDYWGQGTSLTVSS (SEQ ID NO: 2065) |
| acid sequence | |
| 2ID11 VL amino | DIQMTQSPASLSVSVGETVTITCRASENIYSNLAWYQQKQGRSPQLLV |
| acid sequence | YAATNLADGVPSRFSGSGSGTQYSLKINSLQSEDFGYYYCQHFWGTP |
| | YTFGGGTKVEIK (SEQ ID NO: 2066) |
| 21D4.D1 VL amino | DVVMTQTPLTLSVTIGQPASFSCKSSQSLLDSDGKTYLNWLLRRPGQS |
| acid sequence | PKRLIYVVSKLDSGVPDRFTGSGSGTDFTLKISRVEAEDLGVYYCWQG |
| | THFPYTFGGGTKLEIK (SEQ ID NO: 2067) |
| 26D2 VL amino acid | DIQMTQSSSSFSVSLGDRVTITCKASEDIYNRLAWYQQKPGNAPRLLIS |
| sequence | GATSLETGVPSRFSGSGSGKDYTLSITSLQTEDVATYYCQQYWSTPWT |
| | FGGGTKLEIK (SEQ ID NO: 2068) |
| 26E2.A3 VL amino | DVVMTQTPLSLPVSLGDQASISCRSSQSLVHINGNTYLQWFLQKPGQS |
| acid sequence; | PKLLIYKVSNRFSGVPDRFSGSGSGTAFTLKISRVEAEDLGVYFCSQST |
| 24B4.A1 VL amino | HVPYTFGGGTKLEIK (SEQ ID NO: 2069) |
| acid sequence | |
| 3D3.A1 VL amino | DIVMSQSPSSLAVSVGEKVTMSCKSSQSLLYSSNQKSYLAWYQQKPG |
| acid sequence | QSPKLLIYWASTRESGVPDRFRGSGSGTDFTLTISSVKAEDLAVYYCQ |
| | QYFSYPPTFGGGTKLEIK (SEQ ID NO: 2070) |
| 40H3.A4 VL amino | DIVMTQAAFSNPVTLGTSASISCRSSKSLLHSNGITYLYWYLQKPGQSP |
| acid sequence | QLLIYQMSNLASGVPDRFSSSGSGIDFTLRINRVEAEDVGVYYCAQNL |
| | ELPTFGSGTKLEIK (SEQ ID NO: 2071) |
| 42E8.H1 VL amino | DVVMTQNPLSLPVSLGDQASISCRSSQSLVHINGNTYLHWYLQKPGQS |
| acid sequence | PKWYKVSNRFSGVPDRFSGSGSGTDFTLKISRVEAEDLGVYFCSQTT |
| | HALFTFGSGTKLEIK (SEQ ID NO: 2072) |
| 49H1 LB 1 VL amino | DVVMTQTPLSLPVSLGDQASISCRSSQSLVHINGNTYLHWYLQKPGQS |
| acid sequence | PKLLIYKVSNRFSGVPDRFSGSGSGTDFTLKISRVEAEDLGVYFCSQST |
| | HVTFTFGSGTKLEIK (SEQ ID NO: 2073) |
| 54C2.A1 VL amino | DVVMTQTPLSLPVSLGDQASISCRSSQSLVHINGNTYLQWYLQKPGQS |
| acid | PKLLIYKVSNRFSGVPDRFSGSGSGTDFTLRISRVEAEDLGVYFCSQST |
| | HLPYTFGGGTKLEIK (SEQ ID NO: 2074) |
| 57D7.A1 VL amino | DVLMTQTPLSLPVSLGDQASISCRSSQSIVHSNGNTYLEWYLQKPGQS |
| acid sequence | PKWYKVSNRFSGVPDRFSGSGSGTDFTLKISRVEAEDLGVYYCFQGS |
| | HVPYTFGGGTKLEIK (SEQ ID NO: 2075) |
| RS9.F6 VL amino | DVVMTQTPLSLPVSLGDQASISCRSSQSLVHNNGNTFLHWYLQKPGQ |
| acid sequence; | SPKWYKVSNRFSGVPDRFSGSGSGTDFTLKISRVEAEDLGVYFCSQT |
| RS.F10 VL amino | THVPPTFGGGTKLEIK (SEQ ID NO: 2076) |
| acid sequence | |
| RS9.F6 and RS.F10 | GYTFTSYWMH (SEQ ID NO: 2077) |
| CDR-H1 | |
| R59.F6 and RS.F10 | RSDPTTGGTNYNEKFKT (SEQ ID NO: 2078) |
| CDR-H2 | |
| RS9.F6. RS.F10, | KVSNRFS (SEQ ID NO: 2079) |
| 26E2.A3, 24B4.A1, | |
| 42E8.H1, 49H11.B1, | |
| 54C2.A1, and | |
| 57D7.A1 CDR-L2 | |
| 2ID11 CDR-H1 | GYTFTSYWIQ (SEQ ID NO: 2080) |
| 2ID11 CDR-H2 | TIYPGDGDARYTQKFKG (SEQ ID NO: 2081) |
| 2ID11 CDR-H3 | ARNGITTAGYYAMDY (SEQ ID NO: 2082) |
| 2ID11 CDR-L1 | RASENIYSNLA (SEQ ID NO: 2083) |
| 2ID11 CDR-L2 | AATNLAD (SEQ ID NO: 2084) |
| 2ID11 CDR-L3 | QHFWGTPYT (SEQ ID NO: 2085) |
| 21D4.D1 CDR-H1 | GYTFTDHAMH (SEQ ID NO: 2086) |
| 21D4.D1 CDR-H2 | VISTYSGDTGYNQKFKG (SEQ ID NO: 2087) |
| 21D4.D1 CDR-H3 | AREGHYDDAMDY (SEQ ID NO: 2088) |
| 21D4.D1 and 51D4 | KSSQSLLDSDGKTYLN (SEQ ID NO: 2089) |
| CDR-L1 | |
| 21D4.D1 CDR-L2 | VVSKLDS (SEQ ID NO: 2090) |
| 21D4.D1 and 51D4 | WQGTFIFPYT (SEQ ID NO: 2091) |
| CDR-L3 | |
| 26D2 CDR-H1 | GYTFTSYVMH (SEQ ID NO: 2092) |
| 26D2 CDR-H2 | YINPYTDGTKYNEKFKG (SEQ ID NO: 2093) |
| 26D2 CDR-H3 | ARGEVRRYALDY (SEQ ID NO: 2094) |
| 26D2 CDR-L1 | KASEDIYNRLA (SEQ ID NO: 2095) |
| 26D2 CDR-L2 | GATSLET (SEQ ID NO: 2096) |
| 26D2 CDR-L3 | QQYWSTPWT (SEQ ID NO: 2097) |
| 26E2.A3 and | DSEVFPISYMS (SEQ ID NO: 2098) |
| 24B4.A1 CDR-H1 | |
| 26E2.A3 and | DILPSIGGRIYGVKF (SEQ ID NO: 2099) |
| 24B4.A1 CDR-H2 | |
| 26E2.A3 and | ARKDYGSLAY (SEQ ID NO: 2100) |
| 24B4.A1 CDR-H3 | |
| 26E2.A3, 24B4.A1, | RSSQSLVHINGNTYLQ (SEQ ID NO: 2101) |
| and 54C2.A1 CDR-Ll | |
| 26E2.A3 and | SQSTHVPYT (SEQ ID NO: 2102) |
| 24B4.A1 CDR-L3 | |
| 3D3.A1 CDR-H1 | GYTLSEYTMH (SEQ ID NO: 2103) |
| 3D3.A1 CDR-H2 | GVIPNSGGTSYNQKFRD (SEQ ID NO: 2104) |
| 3D3.A1 CDR-H3 | ARGDDSYRRGYALDY (SEQ ID NO: 2105) |
| 3D3.A1 CDR-L1 | KSSQSLLYSSNQKSYLA (SEQ ID NO: 2106) |
| 3D3.A1 CDR-L2 | WASTRES (SEQ ID NO: 2107) |
| 3D3.A1 CDR-L3 | QQYFSYPPT (SEQ ID NO: 2108) |
| 40H3.A4 CDR-H1 | GFNIKDTYMH (SEQ ID NO: 2109) |
| 40H3.A4 CDR-H2 | RIDPANGNTKYDPKFQG (SEQ ID NO: 2110) |
| 40H3.A4 CDR-H3 | ATLFAY (SEQ ID NO: 2111) |
| 40H3.A4 CDR-L1 | RSSKSLLHSNGITYLY (SEQ ID NO: 2112) |
| 40H3.A4 CDR-L2 | QMSNLAS (SEQ ID NO: 2113) |
| 40H3.A4 CDR-L3 | AQNLELPT (SEQ ID NO: 2114) |
| 42E8.H1 and 49H11.B1 | GYSITSDYAWN (SEQ ID NO: 2115) |
| CDR-H1 | |
| 42E8.H1 CDR-H2 | YINYSGRTIYNPSLKS (SEQ ID NO: 2116) |
| 42E8.H1 and 49H11.B1 | ARWNGNYGFAY (SEQ ID NO: 2117) |
| CDR-H3 | |
| 42E8.H1 and 49H11.B1 | RSSQSLVHINGNTYLH (SEQ ID NO: 2118) |
| CDR-L1 | |
| 42E8.H1 CDR-L3 | SQTTHALFT (SEQ ID NO: 2119) |
| 49H11.B1 CDR-H2 | YISFSGSTSYNPSLKS (SEQ ID NO: 2120) |
| 49H11.B1 CDR-L3 | SQSTHVTFT (SEQ ID NO: 2121) |
| 54C2.A1 CDR-H1 | DSEVFPIAYMS (SEQ ID NO: 2122) |
| 54C2.A1 CDR-H2 | DILPSIGRRIYGVKFED (SEQ ID NO: 2123) |
| 54C2.A1 CDR-H3 | KDYGSLAY (SEQ ID NO: 2124) |
| 54C2.A1 CDR-L3 | SQSTHLPYT (SEQ ID NO: 2125) |
| 57D7.A1 CDR-H1 | GFSLSRYSVY (SEQ ID NO: 2126) |
| 57D7.A1 CDR-H2 | MIWGGGNTDYNSALKS (SEQ ID NO: 2127) |
| 57D7.A1 CDR-H3 | YGGMDY (SEQ ID NO: 2128) |
| 57D7.A1 CDR-L1 | RSSQSIVHSNGNTYLE (SEQ ID NO: 2129) |
| 57D7.A1 CDR-L3 | FQGSHVPYT (SEQ ID NO: 2130) |
| RS9.F6-Fd | QVQLQQPGAELVKPGASVKLSCKASGYTFTSYWMHWVKQSPGRGLE |
| | WIGRSDPTTGGTNYNEKFKTKATLTVDKPSSTAYMQLSSLTSDDSAV |
| | YYCVRTSGTGDYWGQGTSLTVSSASTKGPSVFPLAPSSKSTSGGTAAL |
| | GCLVKDYFPEPVTVSWNSGALTSGVHTFPAVLQSSGLYSLSSVVTVPS |
| | SSLGTQTYICNVNHKPSNTKVDKKVEPKSCDKTH (SEQ ID NO: 2131) |
| RS9.F6-Fd fused to | QVQLQQPGAELVKPGASVKLSCKASGYTFTSYWMHWVKQSPGRGLE |
| Fc with LALAPG, | WIGRSDPTTGGTNYNEKFKTKATLTVDKPSSTAYMQLSSLTSDDSAV |
| TfR binding, and | YYCVRTSGTGDYWGQGTSLTVSSASTKGPSVFPLAPSSKSTSGGTAAL |
| knob mutations | GCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPEAAGGP |
| | SVFLFPPKPKDTLMISRTPEVTCVVVDVSHEDPEVKFNWYVDGVEVH |
| | NAKTKPREEQYNSTYRVVSVLTVLHQDWLNGKEYKCKVSNKALGAP |
| | IEKTISKAKGQPREPQVYTLPPSRDELTKNQVSLWCLVKGFYPSDIAVL |
| | WESYGTEWASYKTTPPVLDSDGSFFLYSKLTVTKEEWQQGFVFSCSV |
| | MHEALHNHYTQKSLSLSPGK (SEQ ID NO: 2132) |
| RS9.F6-Fd fused to | QVQLQQPGAELVKPGASVKLSCKASGYTFTSYWMHWVKQSPGRGLE |
| Fc with LALAPG and | WIGRSDPTTGGTNYNEKFKTKATLTVDKPSSTAYMQLSSLTSDDSAV |
| hole mutations | YYCVRTSGTGDYWGQGTSLTVSSASTKGPSVFPLAPSSKSTSGGTAAL |
| | GCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPEAAGGP |
| | SVFLFPPKPKDTLMISRTPEVTCVVVDVSHEDPEVKFNWYVDGVEVH |
| | NAKTKPREEQYNSTYRVVSVLTVLHQDWLNGKEYKCKVSNKALGAP |
| | IEKTISKAKGQPREPQVYTLPPSRDELTKNQVSLSCAVKGFYPSDIAVE |
| | WESNGQPENNYKTTPPVLDSDGSFFLVSKLTVDKSRWQQGNVFSCSV |
| | MHEALHNHYTQKSLSLSPGK (SEQ ID NO: 2133) |
| 3D3.A1-Fd | EVQLQQSGPELVKPGASVKISCKTSGYTLSEYTMHWVIQSHGKSLEWI |
| | GGVIPNSGGTSYNQKFRDKASLTVDKSSSTAYLELRSLTSEDSAVYYC |
| | ARGDDSYRRGYALDYWGQGTSVTVSSASTKGPSVFPLAPSSKSTSGG |
| | TAALGCLVKDYFPEPVTVSWNSGALTSGVHTFPAVLQSSGLYSLSSVV |
| | TVPSSSLGTQTYICNVNHKPSNTKVDKKVEPKSCDKTH |
| | (SEQ ID NO: 2134) |
| 3D3.A1-Fd fused to | EVQLQQSGPELVKPGASVKISCKTSGYTLSEYTMHWVIQSHGKSLEWI |
| Fc with LALAPG, | GGVIPNSGGTSYNQKFRDKASLTVDKSSSTAYLELRSLTSEDSAVYYC |
| TfR binding, and | ARGDDSYRRGYALDYWGQGTSVTVSSASTKGPSVFPLAPSSKSTSGG |
| knob mutations | TAALGCLVKDYFPEPVTVSWNSGALTSGVHTFPAVLQSSGLYSLSSVV |
| | TVPSSSLGTQTYICNVNHKPSNTKVDKKVEPKSCDKTHTCPPCPAPEA |
| | AGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNK |
| | ALGAPIEKTISKAKGQPREPQVYTLPPSRDELTKNQVSLWCLVKGFYP |
| | SDIAVLWESYGTEWASYKTTPPVLDSDGSFFLYSKLTVTKEEWQQGF |
| | VFSCSVMHEALHNHYTQKSLSLSPGK (SEQ ID NO: 2135) |
| 3D3.A1-Fd fused to | EVQLQQSGPELVKPGASVKISCKTSGYTLSEYTMHWVIQSHGKSLEWI |
| Fc with LALAPG and | GGVIPNSGGTSYNQKFRDKASLTVDKSSSTAYLELRSLTSEDSAVYYC |
| hole mutations | ARGDDSYRRGYALDYWGQGTSVTVSSASTKGPSVFPLAPSSKSTSGG |
| | TAALGCLVKDYFPEPVTVSWNSGALTSGVHTFPAVLQSSGLYSLSSVV |
| | TVPSSSLGTQTYICNVNHKPSNTKVDKKVEPKSCDKTHTCPPCPAPEA |
| | AGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNK |
| | ALGAPIEKTISKAKGQPREPQVYTLPPSRDELTKNQVSLSCAVKGFYPS |
| | DIAVEWESNGQPENNYKTTPPVLDSDGSFFLVSKLTVDKSRWQQGNV |
| | FSCSVMHEALHNHYTQKSLSLSPGK (SEQ ID NO: 2136) |
| Human TREM2 | MEPLRLLILLFVTELSGAHNTTVFQGVAGQSLQVSCPYDSMKH |
| protein | WGRRKAWCRQLGEKGPCQRVVSTHNLWLLSFLRRWNGSTAIT |
| | DDTLGGTLTITLRNLQPHDAGLYQCQSLHGSEADTLRKVLVEVL |
| | ADPLDHRDAGDLWFPGESESFEDAHVEHSISRSLLEGEIPFPPTSI |
| | LLLLACIFLIKILAASALWAAAWHGQKPGTHPPSELDCGHDPGY |
| | QLQTLPGLRDT (SEQ ID NO: 1) |
| Human transferrin | MMDQARSAFSNLFGGEPLSYTRFSLARQVDGDNSHVEMKLAVDEEE |
| receptor protein 1 | NADNNTKANVTKPKRCSGSICYGTIAVIVFFLIGFMIGYLGYCKGVEP |
| (TFR1) | KTECERLAGTESPVREEPGEDFPAARRLYWDDLKRKLSEKLDSTDFTG |
| | TIKLLNENSYVPREAGSQKDENLALYVENQFREFKLSKVWRDQHFVK |
| | IQVKDSAQNSVIIVDKNGRLVYLVENPGGYVAYSKAATVTGKLVHAN |
| | FGTKKDFEDLYTPVNGSIVIVRAGKITFAEKVANAESLNAIGVLIYMD |
| | QTKFPIVNAELSFFGHAHLGTGDPYTPGFPSFNHTQFPPSRSSGLPNIPV |
| | QTISRAAAEKLFGNMEGDCPSDWKTDSTCRMVTSESKNVKLTVSNVL |
| | KEIKILNIFGVIKGFVEPDHYVVVGAQRDAWGPGAAKSGVGTALLLK |
| | LAQMFSDMVLKDGFQPSRSIIFASWSAGDFGSVGATEWLEGYLSSLHL |
| | KAFTYINLDKAVLGTSNFKVSASPLLYTLIEKTMQNVKHPVTGQFLYQ |
| | DSNWASKVEKLTLDNAAFPFLAYSGIPAVSFCFCEDTDYPYLGTTMD |
| | TYKELIERIPELNKVARAAAEVAGQFVIKLTHDVELNLDYERYNSQLL |
| | SFVRDLNQYRADIKEMGLSLQWLYSARGDFFRATSRLTTDFGNAEKT |
| | DRFVMKKLNDRVMRVEYHFLSPYVSPKESPFRHVFWGSGSHTLPALL |
| | ENLKLRKQNNGAFNETLFRNQLALATWTIQGAANALSGDVWDIDNE |
| | F (SEQ ID NO: 2137) |
| Wild-type human Fc | APELLGGPSVFLFPPKPKDTLMISRTPEVTCVVVDVSHEDPEVKFNWY |
| sequence posifions | VDGVEVHNAKTKPREEQYNSTYRVVSVLTVLHQDWLNGKEYKCKV |
| 231-447 EU index | SNKALPAPIEKTISKAKGQPREPQVYTLPPSRDELTKNQVSLTCLVKGF |
| numbering | YPSDIAVEWESNGQPENNYKTTPPVLDSDGSFFLYSKLTVDKSRWQQ |
| | GNVFSCSVMHEALHNHYTQKSLSLSPGK (SEQ ID NO: 2138) |
| Human IgG1 hinge | EPKSCDKTHTCPPCP (SEQ ID NO: 2139) |
| sequence | |
| Clone CH3C.35.20 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWSSYKTTPPVLDSDGSFFLYSKLTVTKEEWQQ |
| | GFVFSCSVMHEALHNHYTQKSLSLSPGK (SEQ ID NO: 2140) |
| Clone CH3C.35.21 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWSSYKTTPPVLDSDGSFFLYSKLTVTKEEWQQ |
| | GFVFSCSVMHEALHNHYTQKSLSLSPGK (SEQ ID NO: 2141) |
| Clone CH3C.35.22 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWSNYKTTPPVLDSDGSFFLYSKLTVTKSEWQQ |
| | GFVFSCSVMHEALHNHYTQKSLSLSPGK (SEQ ID NO: 2142) |
| Clone CH3C.35.23 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWSNYKTTPPVLDSDGSFFLYSKLTVTKEEWQQ |
| | GFVFSCSVMHEALHNHYTQKSLSLSPGK (SEQ ID NO: 2143) |
| Clone CH3C.35.24 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWSNYKTTPPVLDSDGSFFLYSKLTVTKEEWQQ |
| | GFVFSCSVMHEALHNHYTQKSLSLSPGK (SEQ ID NO: 2144) |
| Clone CH3C.35.21.17 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWSSYKTTPPVLDSDGSFFLYSKLTVTKEEWQQ |
| | GFVFSCSVMHEALHNHYTQKSLSLSPGK (SEQ ID NO: 2145) |
| Clone CH3C.35.20.1 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESFGTEWSSYKTTPPVLDSDGSFFLYSKLTVTKEEWQQG |
| | FVFSCSVMHEALHNHYTQKSLSLSPGK (SEQ ID NO: 2146) |
| Clone CH3C.35.20.2 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWASYKTTPPVLDSDGSFFLYSKLTVTKEEWQQ |
| | GFVFSCSVMHEALHNHYTQKSLSLSPGK (SEQ ID NO: 2147) |
| Clone CH3C.35.20.3 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWVSYKTTPPVLDSDGSFFLYSKLTVTKEEWQQ |
| | GFVFSCSVMHEALHNHYTQKSLSLSPGK (SEQ ID NO: 2148) |
| Clone CH3C.35.20.4 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWSSYKTTPPVLDSDGSFFLYSKLTVSKEEWQQG |
| | FVFSCSVMHEALHNHYTQKSLSLSPGK (SEQ ID NO: 2149) |
| Clone CH3C.35.20.5 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESFGTEWASYKTTPPVLDSDGSFFLYSKLTVTKEEWQQ |
| | GFVFSCSVMHEALHNHYTQKSLSLSPGK (SEQ ID NO: 2150) |
| Clone CH3C.35.20.6 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESFGTEWVSYKTTPPVLDSDGSFFLYSKLTVTKEEWQQ |
| | GFVFSCSVMHEALHNHYTQKSLSLSPGK (SEQ ID NO: 2151) |
| Clone CH3C.35.21.a.1 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESFGTEWSSYKTTPPVLDSDGSFFLYSKLTVTKEEWQQ |
| | GFVFSCSVMHEALHNHYTQKSLSLSPGK (SEQ ID NO: 2152) |
| Clone | APELLGGPSVFLFPPKPKDTLMISRTPEVTCVVVDVSHEDPEVKFNWY |
| CH3C.35.21.a.2 | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWASYKTTPPVLDSDGSFFLYSKLTVTKEEWQQ |
| | GFVFSCSVMHEALHNHYTQKSLSLSPGK (SEQ ID NO: 2153) |
| Clone | APELLGGPSVFLFPPKPKDTLMISRTPEVTCVVVDVSHEDPEVKFNWY |
| CH3C.35.21.a.3 | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWVSYKTTPPVLDSDGSFFLYSKLTVTKEEWQQ |
| | GFVFSCSVMHEALHNHYTQKSLSLSPGK (SEQ ID NO: 2154) |
| Clone | APELLGGPSVFLFPPKPKDTLMISRTPEVTCVVVDVSHEDPEVKFNWY |
| CH3C.35.21.a.4 | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWSSYKTTPPVLDSDGSFFLYSKLTVSKEEWQQ |
| | GFVFSCSVMHEALHNHYTQKSLSLSPGK (SEQ ID NO: 2155) |
| Clone | APELLGGPSVFLFPPKPKDTLMISRTPEVTCVVVDVSHEDPEVKFNWY |
| CH3C.35.21.a.5 | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESFGTEWASYKTTPPVLDSDGSFFLYSKLTVTKEEWQQ |
| | GFVFSCSVMHEALHNHYTQKSLSLSPGK (SEQ ID NO: 2156) |
| Clone | APELLGGPSVFLFPPKPKDTLMISRTPEVTCVVVDVSHEDPEVKFNWY |
| CH3C.35.21.a.6 | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESFGTEWVSYKTTPPVLDSDGSFFLYSKLTVTKEEWQQ |
| | GFVFSCSVMHEALHNHYTQKSLSLSPGK (SEQ ID NO: 2157) |
| Clone CH3C.35.23.1 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESFGTEWSNYKTTPPVLDSDGSFFLYSKLTVTKEEWQQ |
| | GFVFSCSVMHEALHNHYTQKSLSLSPGK (SEQ ID NO: 2158) |
| Clone CH3C.35.23.2 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWANYKTTPPVLDSDGSFFLYSKLTVTKEEWQQ |
| | GFVFSCSVMHEALHNHYTQKSLSLSPGK (SEQ ID NO: 2159) |
| Clone CH3C.35.23.3 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWVNYKTTPPVLDSDGSFFLYSKLTVTKEEWQQ |
| | GFVFSCSVMHEALHNHYTQKSLSLSPGK (SEQ ID NO: 2160) |
| Clone CH3C.35.23.4 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWSNYKTTPPVLDSDGSFFLYSKLTVSKEEWQQ |
| | GFVFSCSVMHEALHNHYTQKSLSLSPGK (SEQ ID NO: 2161) |
| Clone CH3C.35.23.5 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESFGTEWANYKTTPPVLDSDGSFFLYSKLTVTKEEWQQ |
| | GFVFSCSVMHEALHNHYTQKSLSLSPGK (SEQ ID NO: 2162) |
| Clone CH3C.35.23.6 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESFGTEWVNYKTTPPVLDSDGSFFLYSKLTVTKEEWQQ |
| | GFVFSCSVMHEALHNHYTQKSLSLSPGK (SEQ ID NO: 2163) |
| Clone CH3C.35.24.1 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESFGTEWSNYKTTPPVLDSDGSFFLYSKLTVTKEEWQQ |
| | GFVFSCSVMHEALHNHYTQKSLSLSPGK (SEQ ID NO: 2164) |
| Clone CH3C.35.24.2 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWANYKTTPPVLDSDGSFFLYSKLTVTKEEWQQ |
| | GFVFSCSVMHEALHNHYTQKSLSLSPGK (SEQ ID NO: 2165) |
| Clone CH3C.35.24.3 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWVNYKTTPPVLDSDGSFFLYSKLTVTKEEWQQ |
| | GFVFSCSVMHEALHNHYTQKSLSLSPGK (SEQ ID NO: 2166) |
| Clone CH3C.35.24.4 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWSNYKTTPPVLDSDGSFFLYSKLTVSKEEWQQ |
| | GFVFSCSVMHEALHNHYTQKSLSLSPGK (SEQ ID NO: 2167) |
| Clone CH3C.35.24.5 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESFGTEWANYKTTPPVLDSDGSFFLYSKLTVTKEEWQQ |
| | GFVFSCSVMHEALHNHYTQKSLSLSPGK (SEQ ID NO: 2168) |
| Clone CH3C.35.24.6 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESFGTEWVNYKTTPPVLDSDGSFFLSKLTVTKEEWQQ |
| | GFVFSCSVMHEALHNHYTQKSLSLSPGK (SEQ ID NO: 2169) |
| Clone CH3C.35.21.17.1 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESFGTEWSSYKTTPPVLDSDGSFFLYSKLTVTKEEWQQG |
| | FVFSCSVMHEALHNHYTQKSLSLSPGK (SEQ ID NO: 2170) |
| Clone CH3C.35.21.17.2 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWASYKTTPPVLDSDGSFFLYSKLTVTKEEWQQ |
| | GFVFSCSVMHEALHNHYTQKSLSLSPGK (SEQ ID NO: 2171) |
| Clone CH3C.35.21.17.3 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWVSYKTTPPVLDSDGSFFLYSKLTVTKEEWQQ |
| | GFVFSCSVMHEALHNHYTQKSLSLSPGK (SEQ ID NO: 2172) |
| Clone CH3C.35.21.17.4 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWSSYKTTPPVLDSDGSFFLYSKLTVSKEEWQQG |
| | FVFSCSVMHEALHNHYTQKSLSLSPGK (SEQ ID NO: 2173) |
| Clone CH3C.35.21.17.5 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESFGTEWASYKTTPPVLDSDGSFFLYSKLTVTKEEWQQ |
| | GFVFSCSVMHEALHNHYTQKSLSLSPGK (SEQ ID NO: 2174) |
| Clone CH3C.35.21.17.6 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESFGTEWVSYKTTPPVLDSDGSFFLYSKLTVTKEEWQQ |
| | GFVFSCSVMHEALHNHYTQKSLSLSPGK (SEQ ID NO: 2175) |
| Clones | APELLGGPSVFLFPPKPKDTLMISRTPEVTCVVVDVSHEDPEVKFNWY |
| CH3C.35.N390 and | VDGVEVHNAKTKPREEQYNSTYRVVSVLTVLHQDWLNGKEYKCKV |
| CH3C.35.N163 | SNKALPAPIEKTISKAKGQPREPQVYTLPPSRDELTKNQVSLTCLVKGF |
| | YPSDIAVEWESYGTEWSNYKTTPPVLDSDGSFFLYSKLTVTKSEWQQ |
| | GFVFSCSVMHEALHNHYTQKSLSLSPGK (SEQ ID NO: 2176) |
| Clone CH3C.1 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGLVWVGYKTTPPVLDSDGSFFLYSKLTVAKSTWQQ |
| | GWVFSCSVMHEALHNHYTQKSLSLSPGK (SEQ ID NO: 2177) |
| Clone CH3C.2 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTVWSHYKTTPPVLDSDGSFFLYSKLTVSKSEWQQ |
| | GYVFSCSVMHEALHNHYTQKSLSLSPGK (SEQ ID NO: 2178) |
| Clone CH3C.3 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWSQYKTTPPVLDSDGSFFLYSKLTVEKSDWQQ |
| | GHVFSCSVMHEALHNHYTQKSLSLSPGK (SEQ ID NO: 2179) |
| Clone CH3C.4 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESVGTPWALYKTTPPVLDSDGSFFLYSKLTVLKSEWQQ |
| | GWVFSCSVMHEALHNHYTQKSLSLSPGK (SEQ ID NO: 2180) |
| Clone CH3C.17 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTVWSKYKTTPPVLDSDGSFFLYSKLTVSKSEWQQ |
| | GFVFSCSVMHEALHNHYTQKSLSLSPGK (SEQ ID NO: 2181) |
| Clone CH3C.18 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWAVYKTTPPVLDSDGSFFLYSKLTVPKSTWQQ |
| | GWVFSCSVMHEALHNHYTQKSLSLSPGK (SEQ ID NO: 2182) |
| Clone CH3C.21 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGLVWVGYKTTPPVLDSDGSFFLYSKLTVPKSTWQQ |
| | GWVFSCSVMHEALHNHYTQKSLSLSPGK (SEQ ID NO: 2183) |
| Clone CH3C.25 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESMGHVWVGYKTTPPVLDSDGSFFLYSKLTVDKSTWQ |
| | QGWVFSCSVMHEALHNHYTQKSLSLSPGK (SEQ ID NO: 2184) |
| Clone CH3C.34 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGLVWVFSKTTPPVLDSDGSFFLYSKLTVPKSTWQQG |
| | WVFSCSVMHEALHNHYTQKSLSLSPGK (SEQ ID NO: 2185) |
| Clone CH3C.35 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWSSYKTTPPVLDSDGSFFLYSKLTVTKSEWQQG |
| | FVFSCSVMHEALHNHYTQKSLSLSPGK (SEQ ID NO: 2186) |
| Clone CH3C.44 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWSNYKTTPPVLDSDGSFFLYSKLTVSKSEWQQ |
| | GFVFSCSVMHEALHNHYTQKSLSLSPGK (SEQ ID NO: 2187) |
| Clone CH3C.51 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWVGYKTTPPVLDSDGSFFLYSKLTVSKSEWQQ |
| | GWVFSCSVMHEALHNHYTQKSLSLSPGK (SEQ ID NO: 2188) |
| Clone CH3C.3.1-3 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWVATKTTPPVLDSDGSFFLYSKLTVPKSTWQQ |
| | GWVFSCSVMHEALHNHYTQKSLSLSPGK (SEQ ID NO: 2189) |
| Clone CH3C.3.1-9 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGPVWVHTKTTPPVLDSDGSFFLYSKLTVPKSTWQQ |
| | GWVFSCSVMHEALHNHYTQKSLSLSPGK (SEQ ID NO: 2190) |
| Clone CH3C.3.2-5 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWVDQKTTPPVLDSDGSFFLYSKLTVPKSTWQQ |
| | GWVFSCSVMHEALHNHYTQKSLSLSPGK (SEQ ID NO: 2191) |
| Clone CH3C.3.2-19 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWVNQKTTPPVLDSDGSFFLYSKLTVPKSTWQQ |
| | GWVFSCSVMHEALHNHYTQKSLSLSPGK (SEQ ID NO: 2192) |
| Clone CH3C.3.2-1 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWVNFKTTPPVLDSDGSFFLYSKLTVPKSTWQQ |
| | GWVFSCSVMHEALHNHYTQKSLSLSPGK (SEQ ID NO: 2193) |
| Clone CH3C.18 | APELLGGPSVFLFPPKPKDTLMISRTPEVTCVVVDVSHEDPEVKFNWY |
| variant | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESLGHVWAVYKTTPPVLDSDGSFFLYSKLTVPKSTWQQ |
| | GWVFSCSVMHEALHNHYTQKSLSLSPGK (SEQ ID NO: 2194) |
| Clone CH3C.18 | APELLGGPSVFLFPPKPKDTLMISRTPEVTCVVVDVSHEDPEVKFNWY |
| variant | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESLGHVWAVYKTTPPVLDSDGSFFLYSKLTVPKSTWQQ |
| | GWVFSCSVMHEALHNHYTQKSLSLSPGK (SEQ ID NO: 2195) |
| Clone CH3C.18 | APELLGGPSVFLFPPKPKDTLMISRTPEVTCVVVDVSHEDPEVKFNWY |
| variant | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVYWESLGHVWAVYKTTPPVLDSDGSFFLYSKLTVPKSTWQQ |
| | GWVFSCSVMHEALHNHYTQKSLSLSPGK (SEQ ID NO: 2196) |
| Clone CH3C.18 | APELLGGPSVFLFPPKPKDTLMISRTPEVTCVVVDVSHEDPEVKFNWY |
| variant | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWAVYQTTPPVLDSDGSFFLYSKLTVPKSTWQQ |
| | GWVFSCSVMHEALHNHYTQKSLSLSPGK (SEQ ID NO: 2197) |
| Clone CH3C.18 | APELLGGPSVFLFPPKPKDTLMISRTPEVTCVVVDVSHEDPEVKFNWY |
| variant | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWAVYFTTPPVLDSDGSFFLYSKLTVPKSTWQQ |
| | GWVFSCSVMHEALHNHYTQKSLSLSPGK (SEQ ID NO: 2198) |
| Clone CH3C.18 | APELLGGPSVFLFPPKPKDTLMISRTPEVTCVVVDVSHEDPEVKFNWY |
| variant | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWAVYHTTPPVLDSDGSFFLYSKLTVPKSTWQQ |
| | GWVFSCSVMHEALHNHYTQKSLSLSPGK (SEQ ID NO: 2199) |
| Clone CH3C.35.13 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESLGHVWAVYKTTPPVLDSDGSFFLYSKLTVPKSTWQQ |
| | GWVFSCSVMHEALHNHYTQKSLSLSPGK (SEQ ID NO: 2200) |
| Clone CH3C.35.14 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWAVYQTTPPVLDSDGSFFLYSKLTVPKSTWQQ |
| | GWVFSCSVMHEALHNHYTQKSLSLSPGK (SEQ ID NO: 2201) |
| Clone CH3C.35.15 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESLGHVWAVYQTTPPVLDSDGSFFLYSKLTVPKSTWQQ |
| | GWVFSCSVMHEALHNHYTQKSLSLSPGK (SEQ ID NO: 2202) |
| Clone CH3C.35.16 | APELLGGPsVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESLGHVWVNQKTTPPVLDSDGSFFLYSKLTVPKSTWQQ |
| | GWVFSCSVMHEALHNHYTQKSLSLSPGK (SEQ ID NO: 2203) |
| Clone CH3C.35.17 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWVNQQTTPPVLDSDGSFFLYSKLTVPKSTWQQ |
| | GWVFSCSVMHEALHNHYTQKSLSLSPGK (SEQ ID NO: 2204) |
| Clone CH3C.35.18 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESLGHVWVNQQTTPPVLDSDGSFFLYSKLTVPKSTWQQ |
| | GWVFSCSVMHEALHNHYTQKSLSLSPGK (SEQ ID NO: 2205) |
| Clone CH3C.35.19 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWSSYKTTPPVLDSDGSFFLYSKLTVTKSEWQQ |
| | GFVFSCSVMHEALHNHYTQKSLSLSPGK (SEQ ID NO: 2206) |
| Clone | APELLGGPSVFLFPPKPKDTLMISRTPEVTCVVVDVSHEDPEVKFNWY |
| CH3C.35.K165Q | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWSSYQTTPPVLDSDGSFFLYSKLTVTKSEWQQG |
| | FVFSCSVMHEALHNHYTQKSLSLSPGK (SEQ ID NO: 2207) |
| Clone | APELLGGPSVFLFPPKPKDTLMISRTPEVTCVVVDVSHEDPEVKFNWY |
| CH3C.35.N163.K165Q | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWSNYQTTPPVLDSDGSFFLYSKLTVTKSEWQQ |
| | GFVFSCSVMHEALHNHYTQKSLSLSPGK (SEQ ID NO: 2208) |
| Clone CH3C.35.21.1 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWSSYKTTPPVLDSDGSFFLYSKLTVTKSEWQQG |
| | FVFSCSVMHEALHNHYTQKSLSLSPGK (SEQ ID NO: 2209) |
| Clone CH3C.35.21.2 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWSSYRTTPPVLDSDGSFFLYSKLTVTKSEWQQG |
| | FVFSCSVMHEALHNHYTQKSLSLSPGK (SEQ ID NO: 2210) |
| Clone CH3C.35.21.3 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWSSYRTTPPVLDSDGSFFLYSKLTVTREEWQQG |
| | FVFSCSVMHEALHNHYTQKSLSLSPGK (SEQ ID NO: 2211) |
| Clone CH3C.35.21.4 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWSSYRTTPPVLDSDGSFFLYSKLTVTGEEWQQG |
| | FVFSCSVMHEALHNHYTQKSLSLSPGK (SEQ ID NO: 2212) |
| Clone CH3C.35.21.5 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWSSYRTTPPVLDSDGSFFLYSKLTVTREEWQQG |
| | FVFSCWVMHEALHNHYTQKSLSLSPGK (SEQ ID NO: 2213) |
| Clone CH3C.35.21.6 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWSSYRTTPPVLDSDGSFFLYSKLTVTKEEWQQG |
| | FVFSCWVMHEALHNHYTQKSLSLSPGK (SEQ ID NO: 2214) |
| Clone CH3C.35.21.7 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWSSYRTTPPVLDSDGSFFLYSKLTVTREEWQQG |
| | FVFTCWVMHEALHNHYTQKSLSLSPGK (SEQ ID NO: 2215) |
| Clone CH3C.35.21.8 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWSSYRTTPPVLDSDGSFFLYSKLTVTREEWQQG |
| | FVFTCGVMHEALHNHYTQKSLSLSPGK (SEQ ID NO: 2216) |
| Clone CH3C.35.21.9 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWSSYRTTPPVLDSDGSFFLYSKLTVTREEWQQG |
| | FVFECWVMHEALHNHYTQKSLSLSPGK (SEQ ID NO: 2217) |
| Clone CH3C.35.21.10 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWSSYRTTPPVLDSDGSFFLYSKLTVTREEWQQG |
| | FVFKCWVMHEALHNHYTQKSLSLSPGK (SEQ ID NO: 2218) |
| Clone CH3C.35.21.il | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWSSYRTTPPVLDSDGSFFLYSKLTVTPEEWQQG |
| | FVFKCWVMHEALHNHYTQKSLSLSPGK (SEQ ID NO: 2219) |
| Clone CH3C.35.21.12 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWSSYRTTPPVLDSDGSFFLYSKLTVTREEWQQ |
| | GFVFSCSVMHEALHNHYTQKSLSLSPGK (SEQ ID NO: 2220) |
| Clone CH3C.35.21.13 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWSSYRTTPPVLDSDGSFFLYSKLTVTGEEWQQ |
| | GFVFSCSVMHEALHNHYTQKSLSLSPGK (SEQ ID NO: 2221) |
| Clone CH3C.35.21.14 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWSSYRTTPPVLDSDGSFFLYSKLTVTREEWQQ |
| | GFVFTCWVMHEALHNHYTQKSLSLSPGK (SEQ ID NO: 2222) |
| Clone CH3C.35.21.15 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWSSYRTTPPVLDSDGSFFLYSKLTVTGEEWQQ |
| | GFVFTCWVMHEALHNHYTQKSLSLSPGK (SEQ ID NO: 2223) |
| Clone CH3C.35.21.16 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVWWESYGTEWSSYRTTPPVLDSDGSFFLYSKLTVTREEWQQ |
| | GFVFTCGVMHEALHNHYTQKSLSLSPGK (SEQ ID NO: 2224) |
| Clone CH3C.35.21.18 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVLWESYGTEWSSYRTTPPVLDSDGSFFLYSKLTVTKEEWQQG |
| | FVFSCSVMHEALHNHYTQKSLSLSPGK (SEQ ID NO: 2225) |
| Clone CH3B.1 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPRFDYVTTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYGFHDLSLSPGK (SEQ ID NO: 2226) |
| Clone CH3B.2 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPRFDMVTTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYGFHDLSLSPGK (SEQ ID NO: 2227) |
| Clone CH3B.3 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPRFEYVTTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYGFHDLSLSPGK (SEQ ID NO: 2228) |
| Clone CH3B.4 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPRFEMVTTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYGFHDLSLSPGK (SEQ ID NO: 2229) |
| Clone CH3B.5 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPRFELVTTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYGFHDLSLSPGK (SEQ ID NO: 2230) |
| Clone CH2A2.1 | APELLGGPSVFLFPPKPKDTLMISRTPEVTCVVVDVSHEDPEVEFIWYV |
| | DGVDVRYEWQLPREEQYNSTYRVVSVLTVLHQDWLNGKEYKCKVS |
| | NKALPAPIEKTISKAKGQPREPQVYTLPPSRDELTKNQVSLTCLVKGFY |
| | PSDIAVEWESNGQPENNYKTTPPVLDSDGSFFLYSKLTVDKSRWQQG |
| | NVFSCSVMHEALHNHYTQKSLSLSPGK (SEQ ID NO: 2231) |
| Clone CH2A2.2 | APELLGGPSVFLFPPKPKDTLMISRTPEVTCVVVDVSHEDPEVGFVWY |
| | VDGVPVSWEWYWPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYTQKSLSLSPGK (SEQ ID NO: 2232) |
| Clone CH2A2.3 | APELLGGPSVFLFPPKPKDTLMISRTPEVTCVVVDVSHEDPEVQFDWY |
| | VDGVMVRREWHRPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYTQKSLSLSPGK (SEQ ID NO: 2233) |
| Clone CH2A2.4 | APELLGGPSVFLFPPKPKDTLMISRTPEVTCVVVDVSHEDPEVSFEWY |
| | VDGVPVRWEWQWPREEQYNSTYRVVSVLTVLHQDWLNGKEYKCK |
| | VSNKALPAPIEKTISKAKGQPREPQVYTLPPSRDELTKNQVSLTCLVKG |
| | FYPSDIAVEWESNGQPENNYKTTPPVLDSDGSFFLYSKLTVDKSRWQ |
| | QGNVFSCSVMHEALHNHYTQKSLSLSPGK (SEQ ID NO: 2234) |
| Clone CH2A2.5 | APELLGGPSVFLFPPKPKDTLMISRTPEVTCVVVDVSHEDPEVAFTWY |
| | VDGVPVRWEWQNPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYTQKSLSLSPGK (SEQ ID NO: 2235) |
| Clone CH2C.1 | APELLGGPSVFLFPPKPKDTLMISRTPEVTCVVVDPQTPPWEVKFNWY |
| | VDGVEVHNAKTKPREEEYYTYYRVVSVLTVLHQDWLNGKEYKCKV |
| | SNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYTQKSLSLSPGK (SEQ ID NO: 2236) |
| Clone CH2C.2 | APELLGGPSVFLFPPKPKDTLMISRTPEVTCVVVDPPSPPWEVKFNWY |
| | VDGVEVHNAKTKPREEEYYSNYRVVSVLTVLHQDWLNGKEYKCKV |
| | SNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYTQKSLSLSPGK (SEQ ID NO: 2237) |
| Clone CH2C.3 | APELLGGPSVFLFPPKPKDTLMISRTPEVTCVVVDPQTPPWEVKFNWY |
| | VDGVEVHNAKTKPREEEYYSNYRVVSVLTVLHQDWLNGKEYKCKV |
| | SNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYTQKSLSLSPGK (SEQ ID NO: 2238) |
| Clone CH2C.4 | APELLGGPSVFLFPPKPKDTLMISRTPEVTCVVVDFRGPPWEVKFNWY |
| | VDGVEVHNAKTKPREEEYYHDYRVVSVLTVLHQDWLNGKEYKCKV |
| | SNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYTQKSLSLSPGK (SEQ ID NO: 2239) |
| Clone CH2C.5 | APELLGGPSVFLFPPKPKDTLMISRTPEVTCVVVDPQTVPWEVKFNWY |
| | VDGVEVHNAKTKPREEEYYSNYRVVSVLTVLHQDWLNGKEYKCKV |
| | SNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYTQKSLSLSPGK (SEQ ID NO: 2240) |
| Clone CH2D.1 | APELLGGPSVFLFPPKPKDTLMISRTPEVTCVVVDVSVPPRMVKFNWY |
| | VDGVEVHNAKTKSLTSQHNSTVRVVSVLTVLHQDWLNGKEYKCKVS |
| | NKALPAPIEKTISKAKGQPREPQVYTLPPSRDELTKNQVSLTCLVKGFY |
| | PSDIAVEWESNGQPENNYKTTPPVLDSDGSFFLYSKLTVDKSRWQQG |
| | NVFSCSVMHEALHNHYTQKSLSLSPGK (SEQ ID NO: 2241) |
| Clone CH2D.2 | APELLGGPSVFLFPPKPKDTLMISRTPEVTCVVVDVSVPPWMVKFNW |
| | YVDGVEVHNAKTKSLTSQHNSTVRVVSVLTVLHQDWLNGKEYKCK |
| | VSNKALPAPIEKTISKAKGQPREPQVYTLPPSRDELTKNQVSLTCLVKG |
| | FYPSDIAVEWESNGQPENNYKTTPPVLDSDGSFFLYSKLTVDKSRWQ |
| | QGNVFSCSVMHEALHNHYTQKSLSLSPGK (SEQ ID NO: 2242) |
| Clone CH2D.3 | APELLGGPSVFLFPPKPKDTLMISRTPEVTCVVVDVSDMWEYVKFNW |
| | YVDGVEVHNAKTKPWVKQLNSTWRVVSVLTVLHQDWLNGKEYKC |
| | KVSNKALPAPIEKTISKAKGQPREPQVYTLPPSRDELTKNQVSLTCLVK |
| | GFYPSDIAVEWESNGQPENNYKTTPPVLDSDGSFFLYSKLTVDKSRW |
| | QQGNVFSCSVMHEALHNHYTQKSLSLSPGK (SEQ ID NO: 2243) |
| Clone CH2D.4 | APELLGGPSVFLFPPKPKDTLMISRTPEVTCVVVDVSDDWTWVKFNW |
| | YVDGVEVHNAKTKPWIAQPNSTWRVVSVLTVLHQDWLNGKEYKCK |
| | VSNKALPAPIEKTISKAKGQPREPQVYTLPPSRDELTKNQVSLTCLVKG |
| | FYPSDIAVEWESNGQPENNYKTTPPVLDSDGSFFLYSKLTVDKSRWQ |
| | QGNVFSCSVMHEALHNHYTQKSLSLSPGK (SEQ ID NO: 2244) |
| Clone CH2D.5 | APELLGGPSVFLFPPKPKDTLMISRTPEVTCVVVDVSDDWEWVKFNW |
| | YVDGVEVHNAKTKPWKLQLNSTWRVVSVLTVLHQDWLNGKEYKCK |
| | VSNKALPAPIEKTISKAKGQPREPQVYTLPPSRDELTKNQVSLTCLVKG |
| | FYPSDIAVEWESNGQPENNYKTTPPVLDSDGSFFLYSKLTVDKSRWQ |
| | QGNVFSCSVMHEALHNHYTQKSLSLSPGK (SEQ ID NO: 2245) |
| Clone CH2E3.1 | APELLGGPSVFLFPPKPKDTLMISRTPEVTCVVVDVSHEDPWVWFYW |
| | YVDGVEVHNAKTKPREEQYNSTYRVVSVLTVLHQDWLNGKEYKCS |
| | VVNIALWWSIEKTISKAKGQPREPQVYTLPPSRDELTKNQVSLTCLVK |
| | GFYPSDIAVEWESNGQPENNYKTTPPVLDSDGSFFLYSKLTVDKSRW |
| | QQGNVFSCSVMHEALHNHYTQKSLSLSPGK (SEQ ID NO: 2246) |
| Clone CH2E3.2 | APELLGGPSVFLFPPKPKDTLMISRTPEVTCVVVDVSHEDPVVGFRWY |
| | VDGVEVHNAKTKPREEQYNSTYRVVSVLTVLHQDWLNGKEYKCRVS |
| | NSALTWKIEKTISKAKGQPREPQVYTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYTQKSLSLSPGK (SEQ ID NO: 2247) |
| Clone CH2E3.3 | APELLGGPSVFLFPPKPKDTLMISRTPEVTCVVVDVSHEDPVVGFRWY |
| | VDGVEVHNAKTKPREEQYNSTYRVVSVLTVLHQDWLNGKEYKCRVS |
| | NSALSWRIEKTISKAKGQPREPQVYTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYTQKSLSLSPGK (SEQ ID NO: 2248) |
| Clone CH2E3.4 | APELLGGPSVFLFPPKPKDTLMISRTPEVTCVVVDVSHEDPIVGFRWY |
| | VDGVEVHNAKTKPREEQYNSTYRVVSVLTVLHQDWLNGKEYKCRVS |
| | NSALRWRIEKTISKAKGQPREPQVYTLPPSRDELTKNQVSLTCLVKGF |
| | YPSDIAVEWESNGQPENNYKTTPPVLDSDGSFFLYSKLTVDKSRWQQ |
| | GNVFSCSVMHEALHNHYTQKSLSLSPGK (SEQ ID NO: 2249) |
| Clone CH2E3.5 | APELLGGPSVFLFPPKPKDTLMISRTPEVTCVVVDVSHEDPAVGFEWY |
| | VDGVEVHNAKTKPREEQYNSTYRVVSVLTVLHQDWLNGKEYKCQV |
| | FNWALDWVIEKTISKAKGQPREPQVYTLPPSRDELTKNQVSLTCLVK |
| | GFYPSDIAVEWESNGQPENNYKTTPPVLDSDGSFFLYSKLTVDKSRW |
| | QQGNVFSCSVMHEALHNHYTQKSLSLSPGK (SEQ ID NO: 2250) |
| Fc sequence with hole | APELLGGPSVFLFPPKPKDTLMISRTPEVTCVVVDVSHEDPEVKFNWY |
| mutations | VDGVEVHNAKTKPREEQYNSTYRVVSVLTVLHQDWLNGKEYKCKV |
| | SNKALPAPIEKTISKAKGQPREPQVYTLPPSRDELTKNQVSLSCAVKGF |
| | YPSDIAVEWESNGQPENNYKTTPPVLDSDGSFFLVSKLTVDKSRWQQ |
| | GNVFSCSVMHEALHNHYTQKSLSLSPGK (SEQ ID NO: 2251) |
| Fc sequence with hole | APEAAGGPSVFLFPPKPKDTLMISRTPEVTCVVVDVSHEDPEVKFNWY |
| and LALA mutations | VDGVEVHNAKTKPREEQYNSTYRVVSVLTVLHQDWLNGKEYKCKV |
| | SNKALPAPIEKTISKAKGQPREPQVYTLPPSRDELTKNQVSLSCAVKGF |
| | YPSDIAVEWESNGQPENNYKTTPPVLDSDGSFFLVSKLTVDKSRWQQ |
| | GNVFSCSVMHEALHNHYTQKSLSLSPGK (SEQ ID NO: 2252) |
| Fc sequence with hole | APELLGGPSVFLFPPKPKDTLYITREPEVTCVVVDVSHEDPEVKFNWY |
| and YTE mutations | VDGVEVHNAKTKPREEQYNSTYRVVSVLTVLHQDWLNGKEYKCKV |
| | SNKALPAPIEKTISKAKGQPREPQVYTLPPSRDELTKNQVSLSCAVKGF |
| | YPSDIAVEWESNGQPENNYKTTPPVLDSDGSFFLVSKLTVDKSRWQQ |
| | GNVFSCSVMHEALHNHYTQKSLSLSPGK (SEQ ID NO: 2253) |
| Fc sequence with | APEAAGGPSVFLFPPKPKDTLYITREPEVTCVVVDVSHEDPEVKFNWY |
| hole, LALA, and YTE | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVEWESNGQPENNYKTTPPVLDSDGSFFLVSKLTVDKSRWQQ |
| | GNVFSCSVMHEALHNHYTQKSLSLSPGK (SEQ ID NO: 2254) |
| Fc sequence with | APELLGGPSVFLFPPKPKDTLMISRTPEVTCVVVDVSHEDPEVKFNWY |
| knob mutation | VDGVEVHNAKTKPREEQYNSTYRVVSVLTVLHQDWLNGKEYKCKV |
| | SNKALPAPIEKTISKAKGQPREPQVYTLPPSRDELTKNQVSLWCLVKG |
| | FYPSDIAVEWESNGQPENNYKTTPPVLDSDGSFFLYSKLTVDKSRWQ |
| | QGNVFSCSVMHEALHNHYTQKSLSLSPGK(SEQ ID NO: 2255) |
| Fc sequence with | APEAAGGPSVFLFPPKPKDTLMISRTPEVTCVVVDVSHEDPEVKFNWY |
| knob and LALA | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLWCLVKG |
| | FYPSDIAVEWESNGQPENNYKTTPPVLDSDGSFFLYSKLTVDKSRWQ |
| | QGNVFSCSVMHEALHNHYTQKSLSLSPGK (SEQ ID NO: 2256) |
| Fc sequence with | APELLGGPSVFLFPPKPKDTLYITREPEVTCVVVDVSHEDPEVKFNWY |
| knob and YTE | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLWCLVKG |
| | FYPSDIAVEWESNGQPENNYKTTPPVLDSDGSFFLYSKLTVDKSRWQ |
| | QGNVFSCSVMHEALHNHYTQKSLSLSPGK (SEQ ID NO: 2257) |
| Fc sequence with | APEAAGGPSVFLFPPKPKDTLYITREPEVTCVVVDVSHEDPEVKFNWY |
| knob, LALA, and | VDGVEVHNAKTKPREEQYNSTYRVVSVLTVLHQDWLNGKEYKCKV |
| YTE mutations | SNKALPAPIEKTISKAKGQPREPQVYTLPPSRDELTKNQVSLWCLVKG |
| | FYPSDIAVEWESNGQPENNYKTTPPVLDSDGSFFLYSKLTVDKSRWQ |
| | QGNVFSCSVMHEALHNHYTQKSLSLSPGK (SEQ ID NO: 2258) |
| Clone CH3C.35.21 | APELLGGPSVFLFPPKPKDTLMISRTPEVTCVVVDVSHEDPEVKFNWY |
| with knob mutation | VDGVEVHNAKTKPREEQYNSTYRVVSVLTVLHQDWLNGKEYKCKV |
| | SNKALPAPIEKTISKAKGQPREPQVYTLPPSRDELTKNQVSLWCLVKG |
| | FYPSDIAVWWESYGTEWSSYKTTPPVLDSDGSFFLYSKLTVTKEEWQ |
| | QGFVFSCSVMHEALHNHYTQKSLSLSPGK (SEQ ID NO: 2259) |
| Clone CH3C.35.21 | APEAAGGPSVFLFPPKPKDTLMISRTPEVTCVVVDVSHEDPEVKFNWY |
| with knob and LALA | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLWCLVKG |
| | FYPSDIAVWWESYGTEWSSYKTTPPVLDSDGSFFLYSKLTVTKEEWQ |
| | QGFVFSCSVMHEALHNHYTQKSLSLSPGK (SEQ ID NO: 2260) |
| | APELLGGPSVFLFPPKPKDTLYITREPEVTCVVVDVSHEDPEVKFNWY |
| Clone CH3C.35.21 | VDGVEVHNAKTKPREEQYNSTYRVVSVLTVLHQDWLNGKEYKCKV |
| with knob and YTE | SNKALPAPIEKTISKAKGQPREPQVYTLPPSRDELTKNQVSLWCLVKG |
| mutations | FYPSDIAVWWESYGTEWSSYKTTPPVLDSDGSFFLYSKLTVTKEEWQ |
| | QGFVFSCSVMHEALHNHYTQKSLSLSPGK (SEQ ID NO: 2261) |
| Clone CH3C.35.21 | APEAAGGPSVFLFPPKPKDTLYITREPEVTCVVVDVSHEDPEVKFNWY |
| with knob, LALA, | VDGVEVHNAKTKPREEQYNSTYRVVSVLTVLHQDWLNGKEYKCKV |
| and YTE mutations | SNKALPAPIEKTISKAKGQPREPQVYTLPPSRDELTKNQVSLWCLVKG |
| | FYPSDIAVWWESYGTEWSSYKTTPPVLDSDGSFFLYSKLTVTKEEWQ |
| | QGFVFSCSVMHEALHNHYTQKSLSLSPGK (SEQ ID NO: 2262) |
| Clone CH3C.35.21 | APELLGGPSVFLFPPKPKDTLMISRTPEVTCVVVDVSHEDPEVKFNWY |
| with hole mutations | VDGVEVHNAKTKPREEQYNSTYRVVSVLTVLHQDWLNGKEYKCKV |
| | SNKALPAPIEKTISKAKGQPREPQVYTLPPSRDELTKNQVSLSCAVKGF |
| | YPSDIAVWWESYGTEWSSYKTTPPVLDSDGSFFLVSKLTVTKEEWQQ |
| | GFVFSCSVMHEALHNHYTQKSLSLSPGK (SEQ ID NO: 2263) |
| Clone CH3C.35.21 | APEAAGGPSVFLFPPKPKDTLMISRTPEVTCVVVDVSHEDPEVKFNWY |
| with hole and LALA | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVWWESYGTEWSSYKTTPPVLDSDGSFFLVSKLTVTKEEWQQ |
| | GFVFSCSVMHEALHNHYTQKSLSLSPGK (SEQ ID NO: 2264) |
| Clone CH3C.35.21 | APELLGGPSVFLFPPKPKDTLYITREPEVTCVVVDVSHEDPEVKFNWY |
| with hole and YTE | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVWWESYGTEWSSYKTTPPVLDSDGSFFLVSKLTVTKEEWQQ |
| | GFVFSCSVMHEALHNHYTQKSLSLSPGK (SEQ ID NO: 2265) |
| Clone CH3C.35.21 | APEAAGGPSVFLFPPKPKDTLYITREPEVTCVVVDVSHEDPEVKFNWY |
| with hole, LALA, and | VDGVEVHNAKTKPREEQYNSTYRVVSVLTVLHQDWLNGKEYKCKV |
| YTE mutations | SNKALPAPIEKTISKAKGQPREPQVYTLPPSRDELTKNQVSLSCAVKGF |
| | YPSDIAVWWESYGTEWSSYKTTPPVLDSDGSFFLVSKLTVTKEEWQQ |
| | GFVFSCSVMHEALHNHYTQKSLSLSPGK (SEQ ID NO: 2266) |
| Clone CH3C.35.20.1 | APELLGGPSVFLFPPKPKDTLMISRTPEVTCVVVDVSHEDPEVKFNWY |
| with knob mutation | VDGVEVHNAKTKPREEQYNSTYRVVSVLTVLHQDWLNGKEYKCKV |
| | SNKALPAPIEKTISKAKGQPREPQVYTLPPSRDELTKNQVSLWCLVKG |
| | FYPSDIAVEWESFGTEWSSYKTTPPVLDSDGSFFLYSKLTVTKEEWQQ |
| | GFVFSCSVMHEALHNHYTQKSLSLSPGK (SEQ ID NO: 2267) |
| Clone CH3C.35.20.1 | APEAAGGPSVFLFPPKPKDTLMISRTPEVTCVVVDVSHEDPEVKFNWY |
| with knob and LALA | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLWCLVKG |
| | FYPSDIAVEWESFGTEWSSYKTTPPVLDSDGSFFLYSKLTVTKEEWQQ |
| | GFVFSCSVMHEALHNHYTQKSLSLSPGK (SEQ ID NO: 2268) |
| Clone CH3C.35.20.1 | APEAAGGPSVFLFPPKPKDTLMISRTPEVTCVVVDVSHEDPEVKFNWY |
| with knob and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLWCLVKG |
| | FYPSDIAVEWESFGTEWSSYKTTPPVLDSDGSFFLYSKLTVTKEEWQQ |
| | GFVFSCSVMHEALHNHYTQKSLSLSPGK (SEQ ID NO: 2269) |
| Clone CH3C.35.20.1 | APELLGGPSVFLFPPKPKDTLYITREPEVTCVVVDVSHEDPEVKFNWY |
| with knob and YTE | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLWCLVKG |
| | FYPSDIAVEWESFGTEWSSYKTTPPVLDSDGSFFLYSKLTVTKEEWQQ |
| | GFVFSCSVMHEALHNHYTQKSLSLSPGK (SEQ ID NO: 2270) |
| Clone CH3C.35.20.1 | APEAAGGPSVFLFPPKPKDTLYITREPEVTCVVVDVSHEDPEVKFNWY |
| with knob, LALA, | VDGVEVHNAKTKPREEQYNSTYRVVSVLTVLHQDWLNGKEYKCKV |
| and YTE mutations | SNKALPAPIEKTISKAKGQPREPQVYTLPPSRDELTKNQVSLWCLVKG |
| | FYPSDIAVEWESFGTEWSSYKTTPPVLDSDGSFFLYSKLTVTKEEWQQ |
| | GFVFSCSVMHEALHNHYTQKSLSLSPGK (SEQ ID NO: 2271) |
| Clone CH3C.35.20.1 | APEAAGGPSVFLFPPKPKDTLYITREPEVTCVVVDVSHEDPEVKFNWY |
| with knob, LALAPG, | VDGVEVHNAKTKPREEQYNSTYRVVSVLTVLHQDWLNGKEYKCKV |
| and YTE mutations | SNKALGAPIEKTISKAKGQPREPQVYTLPPSRDELTKNQVSLWCLVKG |
| | FYPSDIAVEWESFGTEWSSYKTTPPVLDSDGSFFLYSKLTVTKEEWQQ |
| | GFVFSCSVMHEALHNHYTQKSLSLSPGK (SEQ ID NO: 2272) |
| Clone CH3C.35.20.1 | APELLGGPSVFLFPPKPKDTLMISRTPEVTCVVVDVSHEDPEVKFNWY |
| hole mutations | VDGVEVHNAKTKPREEQYNSTYRVVSVLTVLHQDWLNGKEYKCKV |
| | SNKALPAPIEKTISKAKGQPREPQVYTLPPSRDELTKNQVSLSCAVKGF |
| | YPSDIAVEWESFGTEWSSYKTTPPVLDSDGSFFLVSKLTVTKEEWQQG |
| | FVFSCSVMHEALHNHYTQKSLSLSPGK (SEQ ID NO: 2273) |
| CloneCH3C.35.20.1 | APEAAGGPSVFLFPPKPKDTLMISRTPEVTCVVVDVSHEDPEVKFNWY |
| with hole and LALA | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVEWESFGTEWSSYKTTPPVLDSDGSFFLVSKLTVTKEEWQQG |
| | FVFSCSVMHEALHNHYTQKSLSLSPGK (SEQ ID NO: 2274) |
| Clone CH3C.35.20.1 | APEAAGGPSVFLFPPKPKDTLMISRTPEVTCVVVDVSHEDPEVKFNWY |
| with hole and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLSCAVKG |
| | FYPSDIAVEWESFGTEWSSYKTTPPVLDSDGSFFLVSKLTVTKEEWQQ |
| | GFVFSCSVMHEALHNHYTQKSLSLSPGK (SEQ ID NO: 2275) |
| Clone CH3C.35.20.1 | APELLGGPSVFLFPPKPKDTLYITREPEVTCVVVDVSHEDPEVKFNWY |
| with hole and YTE | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVEWESFGTEWSSYKTTPPVLDSDGSFFLVSKLTVTKEEWQQG |
| | FVFSCSVMHEALHNHYTQKSLSLSPGK (SEQ ID NO: 2276) |
| Clone CH3C.35.20.1 | APEAAGGPSVFLFPPKPKDTLYITREPEVTCVVVDVSHEDPEVKFNWY |
| with hole, LALA, and | VDGVEVHNAKTKPREEQYNSTYRVVSVLTVLHQDWLNGKEYKCKV |
| YTE mutations | SNKALPAPIEKTISKAKGQPREPQVYTLPPSRDELTKNQVSLSCAVKGF |
| | YPSDIAVEWESFGTEWSSYKTTPPVLDSDGSFFLVSKLTVTKEEWQQG |
| | FVFSCSVMHEALHNHYTQKSLSLSPGK (SEQ ID NO: 2277) |
| Clone CH3C.35.20.1 | APEAAGGPSVFLFPPKPKDTLYITREPEVTCVVVDVSHEDPEVKFNWY |
| with hole, LALAPG, | VDGVEVHNAKTKPREEQYNSTYRVVSVLTVLHQDWLNGKEYKCKV |
| and YTE mutations | SNKALGAPIEKTISKAKGQPREPQVYTLPPSRDELTKNQVSLSCAVKG |
| | FYPSDIAVEWESFGTEWSSYKTTPPVLDSDGSFFLVSKLTVTKEEWQQ |
| | GFVFSCSVMHEALHNHYTQKSLSLSPGK (SEQ ID NO: 2278) |
| Clone CH3C.35.23.2 | APELLGGPSVFLFPPKPKDTLMISRTPEVTCVVVDVSHEDPEVKFNWY |
| with knob mutation | VDGVEVHNAKTKPREEQYNSTYRVVSVLTVLHQDWLNGKEYKCKV |
| | SNKALPAPIEKTISKAKGQPREPQVYTLPPSRDELTKNQVSLWCLVKG |
| | FYPSDIAVEWESYGTEWANYKTTPPVLDSDGSFFLYSKLTVTKEEWQ |
| | QGFVFSCSVMHEALHNHYTQKSLSLSPGK (SEQ ID NO: 2279) |
| Clone CH3C.35.23.2 | APEAAGGPSVFLFPPKPKDTLMISRTPEVTCVVVDVSHEDPEVKFNWY |
| with knob and LALA | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLWCLVKG |
| | FYPSDIAVEWESYGTEWANYKTTPPVLDSDGSFFLYSKLTVTKEEWQ |
| | QGFVFSCSVMHEALHNHYTQKSLSLSPGK (SEQ ID NO: 2280) |
| Clone CH3C.35.23.2 | APEAAGGPSVFLFPPKPKDTLMISRTPEVTCVVVDVSHEDPEVKFNWY |
| with knob and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLWCLVKG |
| | FYPSDIAVEWESYGTEWANYKTTPPVLDSDGSFFLYSKLTVTKEEWQ |
| | QGFVFSCSVMHEALHNHYTQKSLSLSPGK (SEQ ID NO: 2281) |
| Clone CH3C.35.23.2 | APELLGGPSVFLFPPKPKDTLYITREPEVTCVVVDVSHEDPEVKFNWY |
| with knob and YTE | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLWCLVKG |
| | FYPSDIAVEWESYGTEWANYKTTPPVLDSDGSFFLYSKLTVTKEEWQ |
| | QGFVFSCSVMHEALHNHYTQKSLSLSPGK (SEQ ID NO: 2282) |
| Clone CH3C.35.23.2 | APEAAGGPSVFLFPPKPKDTLYITREPEVTCVVVDVSHEDPEVKFNWY |
| with knob, LALA, | VDGVEVHNAKTKPREEQYNSTYRVVSVLTVLHQDWLNGKEYKCKV |
| and YTE mutations | SNKALPAPIEKTISKAKGQPREPQVYTLPPSRDELTKNQVSLWCLVKG |
| | FYPSDIAVEWESYGTEWANYKTTPPVLDSDGSFFLYSKLTVTKEEWQ |
| | QGFVFSCSVMHEALHNHYTQKSLSLSPGK (SEQ ID NO: 2283) |
| Clone CH3C.35.23.2 | APEAAGGPSVFLFPPKPKDTLYITREPEVTCVVVDVSHEDPEVKFNWY |
| with knob, LALAPG, | VDGVEVHNAKTKPREEQYNSTYRVVSVLTVLHQDWLNGKEYKCKV |
| and YTE mutations | SNKALGAPIEKTISKAKGQPREPQVYTLPPSRDELTKNQVSLWCLVKG |
| | FYPSDIAVEWESYGTEWANYKTTPPVLDSDGSFFLYSKLTVTKEEWQ |
| | QGFVFSCSVMHEALHNHYTQKSLSLSPGK (SEQ ID NO: 2284) |
| Clone CH3C.35.23.2 | APELLGGPSVFLFPPKPKDTLMISRTPEVTCVVVDVSHEDPEVKFNWY |
| with hole mutations | VDGVEVHNAKTKPREEQYNSTYRVVSVLTVLHQDWLNGKEYKCKV |
| | SNKALPAPIEKTISKAKGQPREPQVYTLPPSRDELTKNQVSLSCAVKGF |
| | YPSDIAVEWESYGTEWANYKTTPPVLDSDGSFFLVSKLTVTKEEWQQ |
| | GFVFSCSVMHEALHNHYTQKSLSLSPGK (SEQ ID NO: 2285) |
| Clone CH3C.35.23.2 | APEAAGGPSVFLFPPKPKDTLMISRTPEVTCVVVDVSHEDPEVKFNWY |
| with hole and LALA | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVEWESYGTEWANYKTTPPVLDSDGSFFLVSKLTVTKEEWQQ |
| | GFVFSCSVMHEALHNHYTQKSLSLSPGK (SEQ ID NO: 2286) |
| Clone CH3C.35.23.2 | APEAAGGPSVFLFPPKPKDTLMISRTPEVTCVVVDVSHEDPEVKFNWY |
| with hole and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLSCAVKG |
| | FYPSDIAVEWESYGTEWANYKTTPPVLDSDGSFFLVSKLTVTKEEWQ |
| | QGFVFSCSVMHEALHNHYTQKSLSLSPGK (SEQ ID NO: 2287) |
| Clone CH3C.35.23.2 | APELLGGPSVFLFPPKPKDTLYITREPEVTCVVVDVSHEDPEVKFNWY |
| with hole and YTE | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVEWESYGTEWANYKTTPPVLDSDGSFFLVSKLTVTKEEWQQ |
| | GFVFSCSVMHEALHNHYTQKSLSLSPGK (SEQ ID NO: 2288) |
| Clone CH3C.35.23.2 | APEAAGGPSVFLFPPKPKDTLYITREPEVTCVVVDVSHEDPEVKFNWY |
| with hole, LALA, and | VDGVEVHNAKTKPREEQYNSTYRVVSVLTVLHQDWLNGKEYKCKV |
| YTE mutations | SNKALPAPIEKTISKAKGQPREPQVYTLPPSRDELTKNQVSLSCAVKGF |
| | YPSDIAVEWESYGTEWANYKTTPPVLDSDGSFFLVSKLTVTKEEWQQ |
| | GFVFSCSVMHEALHNHYTQKSLSLSPGK (SEQ ID NO: 2289) |
| Clone CH3C.35.23.2 | APEAAGGPSVFLFPPKPKDTLYITREPEVTCVVVDVSHEDPEVKFNWY |
| with hole, LALAPG, | VDGVEVHNAKTKPREEQYNSTYRVVSVLTVLHQDWLNGKEYKCKV |
| and YTE mutations | SNKALGAPIEKTISKAKGQPREPQVYTLPPSRDELTKNQVSLSCAVKG |
| | FYPSDIAVEWESYGTEWANYKTTPPVLDSDGSFFLVSKLTVTKEEWQ |
| | QGFVFSCSVMHEALHNHYTQKSLSLSPGK (SEQ ID NO: 2290) |
| Clone CH3C.35.23.3 | APELLGGPSVFLFPPKPKDTLMISRTPEVTCVVVDVSHEDPEVKFNWY |
| with knob mutation | VDGVEVHNAKTKPREEQYNSTYRVVSVLTVLHQDWLNGKEYKCKV |
| | SNKALPAPIEKTISKAKGQPREPQVYTLPPSRDELTKNQVSLWCLVKG |
| | FYPSDIAVEWESYGTEWVNYKTTPPVLDSDGSFFLYSKLTVTKEEWQ |
| | QGFVFSCSVMHEALHNHYTQKSLSLSPGK (SEQ ID NO: 2291) |
| Clone CH3C.35.23.3 | APEAAGGPSVFLFPPKPKDTLMISRTPEVTCVVVDVSHEDPEVKFNWY |
| with knob and LALA | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLWCLVKG |
| | FYPSDIAVEWESYGTEWVNYKTTPPVLDSDGSFFLYSKLTVTKEEWQ |
| | QGFVFSCSVMHEALHNHYTQKSLSLSPGK (SEQ ID NO: 2292) |
| Clone CH3C.35.23.3 | APEAAGGPSVFLFPPKPKDTLMISRTPEVTCVVVDVSHEDPEVKFNWY |
| with knob and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLWCLVKG |
| | FYPSDIAVEWESYGTEWVNYKTTPPVLDSDGSFFLYSKLTVTKEEWQ |
| | QGFVFSCSVMHEALHNHYTQKSLSLSPGK (SEQ ID NO: 2293) |
| Clone CH3C.35.23.3 | APELLGGPSVFLFPPKPKDTLYITREPEVTCVVVDVSHEDPEVKFNWY |
| with knob and YTE | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLWCLVKG |
| | FYPSDIAVEWESYGTEWVNYKTTPPVLDSDGSFFLYSKLTVTKEEWQ |
| | QGFVFSCSVMHEALHNHYTQKSLSLSPGK (SEQ ID NO: 2294) |
| Clone CH3C.35.23.3 | APEAAGGPSVFLFPPKPKDTLYITREPEVTCVVVDVSHEDPEVKFNWY |
| with knob, LALA, | VDGVEVHNAKTKPREEQYNSTYRVVSVLTVLHQDWLNGKEYKCKV |
| and YTE mutations | SNKALPAPIEKTISKAKGQPREPQVYTLPPSRDELTKNQVSLWCLVKG |
| | FYPSDIAVEWESYGTEWVNYKTTPPVLDSDGSFFLYSKLTVTKEEWQ |
| | QGFVFSCSVMHEALHNHYTQKSLSLSPGK (SEQ ID NO: 2295) |
| Clone CH3C.35.23.3 | APEAAGGPSVFLFPPKPKDTLYITREPEVTCVVVDVSHEDPEVKFNWY |
| with knob, LALAPG, | VDGVEVHNAKTKPREEQYNSTYRVVSVLTVLHQDWLNGKEYKCKV |
| and YTE mutations | SNKALGAPIEKTISKAKGQPREPQVYTLPPSRDELTKNQVSLWCLVKG |
| | FYPSDIAVEWESYGTEWVNYKTTPPVLDSDGSFFLYSKLTVTKEEWQ |
| | QGFVFSCSVMHEALHNHYTQKSLSLSPGK (SEQ ID NO: 2296) |
| Clone CH3C.35.23.3 | APELLGGPSVFLFPPKPKDTLMISRTPEVTCVVVDVSHEDPEVKFNWY |
| with hole mutations | VDGVEVHNAKTKPREEQYNSTYRVVSVLTVLHQDWLNGKEYKCKV |
| | SNKALPAPIEKTISKAKGQPREPQVYTLPPSRDELTKNQVSLSCAVKGF |
| | YPSDIAVEWESYGTEWVNYKTTPPVLDSDGSFFLVSKLTVTKEEWQQ |
| | GFVFSCSVMHEALHNHYTQKSLSLSPGK (SEQ ID NO: 2297) |
| Clone CH3C.35.23.3 | APEAAGGPSVFLFPPKPKDTLMISRTPEVTCVVVDVSHEDPEVKFNWY |
| with hole and LALA | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVEWESYGTEWVNYKTTPPVLDSDGSFFLVSKLTVTKEEWQQ |
| | GFVFSCSVMHEALHNHYTQKSLSLSPGK (SEQ ID NO: 2298) |
| Clone CH3C.35.23.3 | APEAAGGPSVFLFPPKPKDTLMISRTPEVTCVVVDVSHEDPEVKFNWY |
| with hole and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLSCAVKG |
| | FYPSDIAVEWESYGTEWVNYKTTPPVLDSDGSFFLVSKLTVTKEEWQ |
| | QGFVFSCSVMHEALHNHYTQKSLSLSPGK (SEQ ID NO: 2299) |
| Clone CH3C.35.23.3 | APELLGGPSVFLFPPKPKDTLYITREPEVTCVVVDVSHEDPEVKFNWY |
| with hole and YTE | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVEWESYGTEWVNYKTTPPVLDSDGSFFLVSKLTVTKEEWQQ |
| | GFVFSCSVMHEALHNHYTQKSLSLSPGK (SEQ ID NO: 2300) |
| Clone CH3C.35.23.3 | APEAAGGPSVFLFPPKPKDTLYITREPEVTCVVVDVSHEDPEVKFNWY |
| with hole. LALA, and | VDGVEVHNAKTKPREEQYNSTYRVVSVLTVLHQDWLNGKEYKCKV |
| YTE mutations | SNKALPAPIEKTISKAKGQPREPQVYTLPPSRDELTKNQVSLSCAVKGF |
| | YPSDIAVEWESYGTEWVNYKTTPPVLDSDGSFFLVSKLTVTKEEWQQ |
| | GFVFSCSVMHEALHNHYTQKSLSLSPGK (SEQ ID NO: 2301) |
| Clone CH3C.35.23.3 | APEAAGGPSVFLFPPKPKDTLYITREPEVTCVVVDVSHEDPEVKFNWY |
| with hole, LALAPG, | VDGVEVHNAKTKPREEQYNSTYRVVSVLTVLHQDWLNGKEYKCKV |
| and YTE mutations | SNKALGAPIEKTISKAKGQPREPQVYTLPPSRDELTKNQVSLSCAVKG |
| | FYPSDIAVEWESYGTEWVNYKTTPPVLDSDGSFFLVSKLTVTKEEWQ |
| | QGFVFSCSVMHEALHNHYTQKSLSLSPGK (SEQ ID NO: 2302) |
| Clone CH3C.35.23.4 | APELLGGPSVFLFPPKPKDTLMISRTPEVTCVVVDVSHEDPEVKFNWY |
| with knob mutation | VDGVEVHNAKTKPREEQYNSTYRVVSVLTVLHQDWLNGKEYKCKV |
| | SNKALPAPIEKTISKAKGQPREPQVYTLPPSRDELTKNQVSLWCLVKG |
| | FYPSDIAVEWESYGTEWSNYKTTPPVLDSDGSFFLYSKLTVSKEEWQQ |
| | GFVFSCSVMHEALHNHYTQKSLSLSPGK (SEQ ID NO: 2303) |
| Clone CH3C.35.23.4 | APEAAGGPSVFLFPPKPKDTLMISRTPEVTCVVVDVSHEDPEVKFNWY |
| with knob and LALA | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLWCLVKG |
| | FYPSDIAVEWESYGTEWSNYKTTPPVLDSDGSFFLYSKLTVSKEEWQQ |
| | GFVFSCSVMHEALHNHYTQKSLSLSPGK (SEQ ID NO: 2304) |
| Clone CH3C.35.23.4 | APEAAGGPSVFLFPPKPKDTLMISRTPEVTCVVVDVSHEDPEVKFNWY |
| with knob and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLWCLVKG |
| | FYPSDIAVEWESYGTEWSNYKTTPPVLDSDGSFFLYSKLTVSKEEWQQ |
| | GFVFSCSVMHEALHNHYTQKSLSLSPGK (SEQ ID NO: 2305) |
| Clone CH3C.35.23.4 | APELLGGPSVFLFPPKPKDTLYITREPEVTCVVVDVSHEDPEVKFNWY |
| with knob and YTE | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLWCLVKG |
| | FYPSDIAVEWESYGTEWSNYKTTPPVLDSDGSFFLYSKLTVSKEEWQQ |
| | GFVFSCSVMHEALHNHYTQKSLSLSPGK (SEQ ID NO: 2306) |
| Clone CH3C.35.23.4 | APEAAGGPSVFLFPPKPKDTLYITREPEVTCVVVDVSHEDPEVKFNWY |
| with knob, LALA, | VDGVEVHNAKTKPREEQYNSTYRVVSVLTVLHQDWLNGKEYKCKV |
| and YTE mutations | SNKALPAPIEKTISKAKGQPREPQVYTLPPSRDELTKNQVSLWCLVKG |
| | FYPSDIAVEWESYGTEWSNYKTTPPVLDSDGSFFLYSKLTVSKEEWQQ |
| | GFVFSCSVMHEALHNHYTQKSLSLSPGK (SEQ ID NO: 2307) |
| Clone CH3C.35.23.4 | APEAAGGPSVFLFPPKPKDTLYITREPEVTCVVVDVSHEDPEVKFNWY |
| with knob, LALAPG, | VDGVEVHNAKTKPREEQYNSTYRVVSVLTVLHQDWLNGKEYKCKV |
| and YTE mutations | SNKALGAPIEKTISKAKGQPREPQVYTLPPSRDELTKNQVSLWCLVKG |
| | FYPSDIAVEWESYGTEWSNYKTTPPVLDSDGSFFLYSKLTVSKEEWQQ |
| | GFVFSCSVMHEALHNHYTQKSLSLSPGK (SEQ ID NO: 2308) |
| Clone CH3C.35.23.4 | APELLGGPSVFLFPPKPKDTLMISRTPEVTCVVVDVSHEDPEVKFNWY |
| with hole mutations | VDGVEVHNAKTKPREEQYNSTYRVVSVLTVLHQDWLNGKEYKCKV |
| | SNKALPAPIEKTISKAKGQPREPQVYTLPPSRDELTKNQVSLSCAVKGF |
| | YPSDIAVEWESYGTEWSNYKTTPPVLDSDGSFFLVSKLTVSKEEWQQ |
| | GFVFSCSVMHEALHNHYTQKSLSLSPGK (SEQ ID NO: 2309) |
| Clone CH3C.35.23.4 | APEAAGGPSVFLFPPKPKDTLMISRTPEVTCVVVDVSHEDPEVKFNWY |
| with hole and LALA | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVEWESYGTEWSNYKTTPPVLDSDGSFFLVSKLTVSKEEWQQ |
| | GFVFSCSVMHEALHNHYTQKSLSLSPGK (SEQ ID NO: 2310) |
| Clone CH3C.35.23.4 | APEAAGGPSVFLFPPKPKDTLMISRTPEVTCVVVDVSHEDPEVKFNWY |
| with hole and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLSCAVKG |
| | FYPSDIAVEWESYGTEWSNYKTTPPVLDSDGSFFLVSKLTVSKEEWQQ |
| | GFVFSCSVMHEALHNHYTQKSLSLSPGK (SEQ ID NO: 2311) |
| Clone CH3C.35.23.4 | APELLGGPSVFLFPPKPKDTLYITREPEVTCVVVDVSHEDPEVKFNWY |
| with hole and YTE | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVEWESYGTEWSNYKTTPPVLDSDGSFFLVSKLTVSKEEWQQ |
| | GFVFSCSVMHEALHNHYTQKSLSLSPGK (SEQ ID NO: 2312) |
| Clone CH3C.35.23.4 | APEAAGGPSVFLFPPKPKDTLYITREPEVTCVVVDVSHEDPEVKFNWY |
| with hole. LALA, and | VDGVEVHNAKTKPREEQYNSTYRVVSVLTVLHQDWLNGKEYKCKV |
| YTE mutations | SNKALPAPIEKTISKAKGQPREPQVYTLPPSRDELTKNQVSLSCAVKGF |
| | YPSDIAVEWESYGTEWSNYKTTPPVLDSDGSFFLVSKLTVSKEEWQQ |
| | GFVFSCSVMHEALHNHYTQKSLSLSPGK (SEQ ID NO: 2313) |
| Clone CH3C.35.23.4 | APEAAGGPSVFLFPPKPKDTLYITREPEVTCVVVDVSHEDPEVKFNWY |
| with hole, LALAPG, | VDGVEVHNAKTKPREEQYNSTYRVVSVLTVLHQDWLNGKEYKCKV |
| and YTE mutations | SNKALGAPIEKTISKAKGQPREPQVYTLPPSRDELTKNQVSLSCAVKG |
| | FYPSDIAVEWESYGTEWSNYKTTPPVLDSDGSFFLVSKLTVSKEEWQQ |
| | GFVFSCSVMHEALHNHYTQKSLSLSPGK (SEQ ID NO: 2314) |
| Clone CH3C.35.21.17.2 | APELLGGPSVFLFPPKPKDTLMISRTPEVTCVVVDVSHEDPEVKFNWY |
| with knob | VDGVEVHNAKTKPREEQYNSTYRVVSVLTVLHQDWLNGKEYKCKV |
| mutation | SNKALPAPIEKTISKAKGQPREPQVYTLPPSRDELTKNQVSLWCLVKG |
| | FYPSDIAVLWESYGTEWASYKTTPPVLDSDGSFFLYSKLTVTKEEWQ |
| | QGFVFSCSVMHEALHNHYTQKSLSLSPGK (SEQ ID NO: 2315) |
| Clone CH3C.35.21.17.2 | APEAAGGPSVFLFPPKPKDTLMISRTPEVTCVVVDVSHEDPEVKFNWY |
| with knob and | VDGVEVHNAKTKPREEQYNSTYRVVSVLTVLHQDWLNGKEYKCKV |
| LALA mutations | SNKALPAPIEKTISKAKGQPREPQVYTLPPSRDELTKNQVSLWCLVKG |
| | FYPSDIAVLWESYGTEWASYKTTPPVLDSDGSFFLYSKLTVTKEEWQ |
| | QGFVFSCSVMHEALHNHYTQKSLSLSPGK (SEQ ID NO: 2316) |
| Clone CH3C.35.21.17.2 | APEAAGGPSVFLFPPKPKDTLMISRTPEVTCVVVDVSHEDPEVKFNWY |
| with knob and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLWCLVKG |
| | FYPSDIAVLWESYGTEWASYKTTPPVLDSDGSFFLYSKLTVTKEEWQ |
| | QGFVFSCSVMHEALHNHYTQKSLSLSPGK (SEQ ID NO: 2317) |
| Clone CH3C.35.21.17.2 | APELLGGPSVFLFPPKPKDTLYITREPEVTCVVVDVSHEDPEVKFNWY |
| with knob and | VDGVEVHNAKTKPREEQYNSTYRVVSVLTVLHQDWLNGKEYKCKV |
| YTE mutations | SNKALPAPIEKTISKAKGQPREPQVYTLPPSRDELTKNQVSLWCLVKG |
| | FYPSDIAVLWESYGTEWASYKTTPPVLDSDGSFFLYSKLTVTKEEWQ |
| | QGFVFSCSVMHEALHNHYTQKSLSLSPGK (SEQ ID NO: 2318) |
| Clone CH3C.35.21.17.2 | APEAAGGPSVFLFPPKPKDTLYITREPEVTCVVVDVSHEDPEVKFNWY |
| with knob, | VDGVEVHNAKTKPREEQYNSTYRVVSVLTVLHQDWLNGKEYKCKV |
| LALA, and YTE | SNKALPAPIEKTISKAKGQPREPQVYTLPPSRDELTKNQVSLWCLVKG |
| mutations | FYPSDIAVLWESYGTEWASYKTTPPVLDSDGSFFLYSKLTVTKEEWQ |
| | QGFVFSCSVMHEALHNHYTQKSLSLSPGK (SEQ ID NO: 2319) |
| Clone CH3C.35.21.17.2 | APEAAGGPSVFLFPPKPKDTLYITREPEVTCVVVDVSHEDPEVKFNWY |
| with knob, | VDGVEVHNAKTKPREEQYNSTYRVVSVLTVLHQDWLNGKEYKCKV |
| LALAPG, and YTE | SNKALGAPIEKTISKAKGQPREPQVYTLPPSRDELTKNQVSLWCLVKG |
| mutations | FYPSDIAVLWESYGTEWASYKTTPPVLDSDGSFFLYSKLTVTKEEWQ |
| | QGFVFSCSVMHEALHNHYTQKSLSLSPGK (SEQ ID NO: 2320) |
| Clone CH3C.35.21.17.2 | APELLGGPSVFLFPPKPKDTLMISRTPEVTCVVVDVSHEDPEVKFNWY |
| with hole | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVLWESYGTEWASYKTTPPVLDSDGSFFLVSKLTVTKEEWQQ |
| | GFVFSCSVMHEALHNHYTQKSLSLSPGK (SEQ ID NO: 2321) |
| Clone CH3C.35.21.17.2 | APEAAGGPSVFLFPPKPKDTLMISRTPEVTCVVVDVSHEDPEVKFNWY |
| with hole and | VDGVEVHNAKTKPREEQYNSTYRVVSVLTVLHQDWLNGKEYKCKV |
| LALA mutations | SNKALPAPIEKTISKAKGQPREPQVYTLPPSRDELTKNQVSLSCAVKGF |
| | YPSDIAVLWESYGTEWASYKTTPPVLDSDGSFFLVSKLTVTKEEWQQ |
| | GFVFSCSVMHEALHNHYTQKSLSLSPGK (SEQ ID NO: 2322) |
| Clone CH3C.35.21.17.2 | APEAAGGPSVFLFPPKPKDTLMISRTPEVTCVVVDVSHEDPEVKFNWY |
| with hole and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLSCAVKG |
| | FYPSDIAVLWESYGTEWASYKTTPPVLDSDGSFFLVSKLTVTKEEWQ |
| | QGFVFSCSVMHEALHNHYTQKSLSLSPGK (SEQ ID NO: 2323) |
| Clone CH3C.35.21.17.2 | APELLGGPSVFLFPPKPKDTLYITREPEVTCVVVDVSHEDPEVKFNWY |
| with hole and | VDGVEVHNAKTKPREEQYNSTYRVVSVLTVLHQDWLNGKEYKCKV |
| YTE mutations | SNKALPAPIEKTISKAKGQPREPQVYTLPPSRDELTKNQVSLSCAVKGF |
| | YPSDIAVLWESYGTEWASYKTTPPVLDSDGSFFLVSKLTVTKEEWQQ |
| | GFVFSCSVMHEALHNHYTQKSLSLSPGK (SEQ ID NO: 2324) |
| Clone CH3C.35.21.17.2 | APEAAGGPSVFLFPPKPKDTLYITREPEVTCVVVDVSHEDPEVKFNWY |
| with hole, | VDGVEVHNAKTKPREEQYNSTYRVVSVLTVLHQDWLNGKEYKCKV |
| LALA, and YTE | SNKALPAPIEKTISKAKGQPREPQVYTLPPSRDELTKNQVSLSCAVKGF |
| mutations | YPSDIAVLWESYGTEWASYKTTPPVLDSDGSFFLVSKLTVTKEEWQQ |
| | GFVFSCSVMHEALHNHYTQKSLSLSPGK (SEQ ID NO: 2325) |
| Clone CH3C.35.21.17.2 | APEAAGGPSVFLFPPKPKDTLYITREPEVTCVVVDVSHEDPEVKFNWY |
| with hole, | VDGVEVHNAKTKPREEQYNSTYRVVSVLTVLHQDWLNGKEYKCKV |
| LALAPG, and YTE | SNKALGAPIEKTISKAKGQPREPQVYTLPPSRDELTKNQVSLSCAVKG |
| mutations | FYPSDIAVLWESYGTEWASYKTTPPVLDSDGSFFLVSKLTVTKEEWQ |
| | QGFVFSCSVMHEALHNHYTQKSLSLSPGK (SEQ ID NO: 2326) |
| Clone CH3C.35.23 | APELLGGPSVFLFPPKPKDTLMISRTPEVTCVVVDVSHEDPEVKFNWY |
| with knob mutation | VDGVEVHNAKTKPREEQYNSTYRVVSVLTVLHQDWLNGKEYKCKV |
| | SNKALPAPIEKTISKAKGQPREPQVYTLPPSRDELTKNQVSLWCLVKG |
| | FYPSDIAVEWESYGTEWSNYKTTPPVLDSDGSFFLYSKLTVTKEEWQ |
| | QGFVFSCSVMHEALHNHYTQKSLSLSPGK (SEQ ID NO: 2327) |
| Clone CH3C.35.23 | APEAAGGPSVFLFPPKPKDTLMISRTPEVTCVVVDVSHEDPEVKFNWY |
| with knob and LALA | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLWCLVKG |
| | FYPSDIAVEWESYGTEWSNYKTTPPVLDSDGSFFLYSKLTVTKEEWQ |
| | QGFVFSCSVMHEALHNHYTQKSLSLSPGK (SEQ ID NO: 2328) |
| Clone CH3C.35.23 | APEAAGGPSVFLFPPKPKDTLMISRTPEVTCVVVDVSHEDPEVKFNWY |
| with knob and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLWCLVKG |
| | FYPSDIAVEWESYGTEWSNYKTTPPVLDSDGSFFLYSKLTVTKEEWQ |
| | QGFVFSCSVMHEALHNHYTQKSLSLSPGK (SEQ ID NO: 2329) |
| Clone CH3C.35.23 | APELLGGPSVFLFPPKPKDTLYITREPEVTCVVVDVSHEDPEVKFNWY |
| with knob and YTE | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLWCLVKG |
| | FYPSDIAVEWESYGTEWSNYKTTPPVLDSDGSFFLYSKLTVTKEEWQ |
| | QGFVFSCSVMHEALHNHYTQKSLSLSPGK (SEQ ID NO: 2330) |
| Clone CH3C.35.23 | APEAAGGPSVFLFPPKPKDTLYITREPEVTCVVVDVSHEDPEVKFNWY |
| with knob, LALA, | VDGVEVHNAKTKPREEQYNSTYRVVSVLTVLHQDWLNGKEYKCKV |
| and YTE mutations | SNKALPAPIEKTISKAKGQPREPQVYTLPPSRDELTKNQVSLWCLVKG |
| | FYPSDIAVEWESYGTEWSNYKTTPPVLDSDGSFFLYSKLTVTKEEWQ |
| | QGFVFSCSVMHEALHNHYTQKSLSLSPGK (SEQ ID NO: 2331) |
| Clone CH3C.35.23 | APEAAGGPSVFLFPPKPKDTLYITREPEVTCVVVDVSHEDPEVKFNWY |
| with knob, LALAPG, | VDGVEVHNAKTKPREEQYNSTYRVVSVLTVLHQDWLNGKEYKCKV |
| and YTE mutations | SNKALGAPIEKTISKAKGQPREPQVYTLPPSRDELTKNQVSLWCLVKG |
| | FYPSDIAVEWESYGTEWSNYKTTPPVLDSDGSFFLYSKLTVTKEEWQ |
| | QGFVFSCSVMHEALHNHYTQKSLSLSPGK (SEQ ID NO: 2332) |
| Clone CH3C.35.23 | APELLGGPSVFLFPPKPKDTLMISRTPEVTCVVVDVSHEDPEVKFNWY |
| with hole mutations | VDGVEVHNAKTKPREEQYNSTYRVVSVLTVLHQDWLNGKEYKCKV |
| | SNKALPAPIEKTISKAKGQPREPQVYTLPPSRDELTKNQVSLSCAVKGF |
| | YPSDIAVEWESYGTEWSNYKTTPPVLDSDGSFFLVSKLTVTKEEWQQ |
| | GFVFSCSVMHEALHNHYTQKSLSLSPGK (SEQ ID NO: 2333) |
| Clone CH3C.35.23 | APEAAGGPSVFLFPPKPKDTLMISRTPEVTCVVVDVSHEDPEVKFNWY |
| with hole and LALA | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVEWESYGTEWSNYKTTPPVLDSDGSFFLVSKLTVTKEEWQQ |
| | GFVFSCSVMHEALHNHYTQKSLSLSPGK (SEQ ID NO: 2334) |
| Clone CH3C.35.23 | APEAAGGPSVFLFPPKPKDTLMISRTPEVTCVVVDVSHEDPEVKFNWY |
| with hole and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLSCAVKG |
| | FYPSDIAVEWESYGTEWSNYKTTPPVLDSDGSFFLVSKLTVTKEEWQ |
| | QGFVFSCSVMHEALHNHYTQKSLSLSPGK (SEQ ID NO: 2335) |
| Clone CH3C.35.23 | APELLGGPSVFLFPPKPKDTLYITREPEVTCVVVDVSHEDPEVKFNWY |
| with hole and YTE | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVEWESYGTEWSNYKTTPPVLDSDGSFFLVSKLTVTKEEWQQ |
| | GFVFSCSVMHEALHNHYTQKSLSLSPGK (SEQ ID NO: 2336) |
| Clone CH3C.35.23 | APEAAGGPSVFLFPPKPKDTLYITREPEVTCVVVDVSHEDPEVKFNWY |
| with hole. LALA, and | VDGVEVHNAKTKPREEQYNSTYRVVSVLTVLHQDWLNGKEYKCKV |
| YTE mutations | SNKALPAPIEKTISKAKGQPREPQVYTLPPSRDELTKNQVSLSCAVKGF |
| | YPSDIAVEWESYGTEWSNYKTTPPVLDSDGSFFLVSKLTVTKEEWQQ |
| | GFVFSCSVMHEALHNHYTQKSLSLSPGK (SEQ ID NO: 2337) |
| Clone CH3C.35.23 | APEAAGGPSVFLFPPKPKDTLYITREPEVTCVVVDVSHEDPEVKFNWY |
| with hole, LALAPG, | VDGVEVHNAKTKPREEQYNSTYRVVSVLTVLHQDWLNGKEYKCKV |
| and YTE mutations | SNKALGAPIEKTISKAKGQPREPQVYTLPPSRDELTKNQVSLSCAVKG |
| | FYPSDIAVEWESYGTEWSNYKTTPPVLDSDGSFFLVSKLTVTKEEWQ |
| | QGFVFSCSVMHEALHNHYTQKSLSLSPGK (SEQ ID NO: 2338) |
| CH3C motif | YxTEWSS (SEQ ID NO: 2339) |
| CH3C motif | TxxExxxxF (SEQ ID NO: 2340) |
| mulgGI 3′ VH PCR | GGACAGGGATCCAGAGTTCC (SEQ ID NO: 2341) |
| primer | |
| mu3/4G2 3′ VH PCR | AGCTGGGAAGGTGTGCACAC (SEQ ID NO: 2342) |
| primer | |
| mu3/4G3 3′ VH PCR | CAGGGGCCAGTGGATAGAC (SEQ ID NO: 2343) |
| primer | |
| muCkappa.1 3′ VL | GACATTGATGTCTTTGGGGT (SEQ ID NO: 2344) |
| PCR primer | |
| muCkappa.2 3′ VL | TTCACTGCCATCAATCTTCC (SEQ ID NO: 2345) |
| PCR primer | |
| 7B10.A2 VH amino | EVQLQQSGPELVKPGASVKMSCKASGYTFTDYNMHWVKQSHGKSLE |
| acid sequence | WIGYINPNNGGTTYNQKFKGKATLTVNKSSSTAYMELRSLTSEDSAV |
| | YYCATYNNHYFDSWGQGTTLTVSS (SEQ ID NO: 2346) |
| 7B10.A2 CDR-H1 | GYTFTDYNMH (SEQ ID NO: 2347) |
| 7B10.A2 CDR-H2 | YINPNNGGTTYNQKFKG (SEQ ID NO: 2348) |
| 7B10.A2 CDR-H3 | ATYNNHYFDS (SEQ ID NO: 2349) |
| 7B10.A2 VL amino | DIQMTQTTSSLSASLGDRVTISCSASQGISNYLNWYQQKPDGTVKLLI |
| acid sequence | YYTSNLHSGVPSRFSGSGSGTDYSLTISNLEPEDIATYYCQQYSNLPYT |
| | FGGGTKLEIK (SEQ ID NO: 2350) |
| 7B10.A2 CDR-L1 | SASQGISNYLN (SEQ ID NO: 2351) |
| 7B10.A2 CDR-L2 | YTSNLHS (SEQ ID NO: 2352) |
| 7B10.A2 CDR-L3 | QQYSNLPYT (SEQ ID NO: 2353) |
| 51D4 VH amino acid | QVHLQQSGPEVVRPGVSVKISCKGSGYTFTDYGMHWVKQSHAKSLE |
| sequence | WIGVISTYNGNTSYNQKYKGKATVTVDKPSSTAYMELVRLTSEDSAI |
| | YYCARDFGYVPFDYWGQGTTLTVSS (SEQ ID NO: 2354) |
| 51D4 CDR-H1 | GYTFTDYGMH (SEQ ID NO: 2355) |
| 51D4 CDR-H2 | VISTYNGNTSYNQKYKG (SEQ ID NO: 2356) |
| 51D4 CDR-H3 | ARDFGYVPFDY (SEQ ID NO: 2357) |
| 51D4 VL amino acid | DVVMTQTPLTLSVTIGQPASISCKSSQSLLDSDGKTYLNWLLQRP |
| sequence | GQSPKRLIYLVSYLDSGVPDRFTGSGSGTDFTLKISRVEADDLGV |
| | YYCWQGTHFPYTFGGGTKLEIK (SEQ ID NO: 2358) |
| 51D4 CDR-L2 | LVSYLDS (SEQ ID NO: 2359) |
| CDR-H1 consensus | GX2X3X4X5X6X7X8X9X10X11, wherein X2 is Y or F; X3 is T, N, or S; X4 is F, |
| sequence | L, or I; X5 is T, S, or K; X6 is D, S, or E; X7 is D or absent; X8 is H, |
| | Y, or T; X9 is A, N, G, V, W, T, or Y; X10 is M, I, or W; and Xis H, Q, or N |
| | (SEQ ID NO: 2360) |
| CDR-H1 consensus | GYX3X4X5X6X7X8X9X10X11, wherein X3 is T or S; X4 is F, L, or I; X5 is |
| sequence | Tor S; X6 is D, S, or E; X7 is D or absent; X8 is H or Y; X9 is A, N, G, V, |
| | W, T, or A; X10 is M, I, or W; and X11 is H, Q, or N (SEQ ID NO: 2361) |
| CDR-H1 consensus | GX2X3X4X5X6X8X9X10X11, wherein X is Y or F; X3 is T or N; X4 is F, L, |
| sequence | or I; X is T, S, or K; X6 is D, S, or E; X8 is H, Y, or T; X is A, N, G, V, |
| | W, T, Y, or A; Xi is M or I; and X is H or Q (SEQ ID NO: 2362) |
| CDR-H1 consensus | GYTX4X5X6X8X9X10X11, wherein X4 is F or L; X is T or S; X6 is D, S, or |
| sequence | E; X8 is H, Y; X9 is A, N, G, V, W, T; X10 is M or I; and X11 is H or Q |
| | (SEQ ID NO: 2363) |
| CDR-H2 consensus | X1X2X3X4X5X6X7X8X9X10YX12X13X14X15X16X17, wherein X1 is D, V, Y, R, |
| sequence | G, or T; X2 is I, S, or V; X3 is L, S, N, D, I, or Y; X4 is P, T, or absent; |
| | X5 is S, Y, N, T, A, G, or F; X6 is I, S, N, T, or D; X7 is G or D; X8 is G, |
| | D, N, R, or S; X9 is R, T, or A; X10 is I, G, S, K, T, N, or R; X12 is G, N, |
| | D, or T; X13 is V, Q, E, or P; X14 is K or S; X15 is F, Y or L; X16 is K, R, |
| | Q, or is absent; and X17 is G, T, D, S, or is absent (SEQ ID NO: 2364) |
| CDR-H2 consensus | X1X2X3X4X5X6X7X8X9X10YX12X13X14X15X16X17, wherein Xi is V, Y, R, G, |
| sequence | or T; X2 is I, S, or V; X3 is S, N, D, I, or Y; X4 is P, T, or absent; X5 |
| | is Y, N, T, A, G, or F; X6 is S, N, T, or D; X7 is G or D; X8 is G, D, N, R, |
| | or S; X9 is T, or A; X10 is I, G, S, K, T, N, or R; X12 is N, D, or T; X13 is |
| | Q, E, or P; X14 is K or 5; X15 is F, Y or L; X16 is K, R, or Q; and Xi is G, T, |
| | D, or S (SEQ ID NO: 2365) |
| CDR-H2 consensus | X1X2X3X4X5X6X7X8X9X10YX12X13KX15X16X17, wherein Xi is V, Y, R, G, |
| sequence | or T; X2 is I, S, or V; X3 is S, N, D, I, or Y ; X4 is P or T; X5 is Y, N, T, |
| | A, or G; X6 is S, N, T, or D; X7 is G or D; X8 is G, D, or N; X9 is T, or A; |
| | X10 is G, S, K, T, N, or R; X12 is N, D, or T; X13 is Q, E, or P; X15 is F or |
| | Y; X16 is K, R, or Q; and X17 is G, T, or D (SEQ ID NO: 2366) |
| CDR-H3 consensus | ARX3X4X5X6X7X8X9X1OYAX13DY, wherein X3 is G or N; X4 is D or G; |
| sequence | X5 is D or I; X6 is S or T; X7 is Y or T; X8 is R or A; X9 is R or G; X10 is G |
| | or Y; and X13 is L or M (SEQ ID NO: 2367) |
| CDR-L1 consensus | X1SSX4SLX7X8X9X10X11X12X13X14X15LX17, wherein X1 is R or K; |
| sequence | X4 is Q or K; X7 is V or L; X8 is H, D, or Y; X9 is I, N, or S; X10 is S or |
| | absent; X11 is D or N; X12 is G or Q; X13 is N, I, or K; X14 is T or S; X15 is |
| | Y or F; and X17 is Q, H, Y, N, or A (SEQ ID NO: 2368) |
| CDR-L1 consensus | X1ASX4X5IX7X8X9LX11, wherein X1 is R, K, or S; X4 is E or Q; X5 is N, |
| sequence | D, or G; X7 is Y or S; X8 is S or N; X9 is N, R, or Y; and X11 is A or N |
| | (SEQ ID NO: 2369) |
| CDR-L2 consensus | X1X2SX4X5X6S, wherein Xi is K, Q, Y, V, or L; X2 is V, M, or T; X4 is N, |
| sequence | K, or Y; X5 is R or L; and X6 is F, A, H, or D (SEQ ID NO: 2370) |
| CDR-L3 consensus | X1X2X3X4X5X6X7X8T, wherein Xi is S, W, or Q; X2 is Q or H; X3 is S, T, |
| sequence | G, Y, or F; X4 is T, F, W, S; X5 is H, S, G, or N; X6 is V, A, F, Y, T, or L; |
| | X7 is P, T, or L; and X8 is Y, F, P, or W (SEQ ID NO: 2371) |
| CDR-L3 consensus | QX2X3X4X5X6PX8T, wherein X2 is Q or H; X3 is Y or F; X4 is F, W, or S; |
| sequence | X5 is S, G, or N; X6 is Y, T, or L; and X8 is P, Y, or W (SEQ ID NO: 2372) |
| Human TREM2 | SGAHNTTVFQGVAGQSLQVSCPYDSMKHWGRRKAWCRQLGEK |
| extracellular domain | GPCQRVVSTHNLWLLSFLRRWNGSTAITDDTLGGTLTITLRNLQP |
| (ECD) amino acid | HDAGLYQCQSLHGSEADTLRKVLVEVLADPLDHRDAGDLWFPG |
| sequence (without | ESESFEDAHVEHSISRSLLEGEIPFPPTS (SEQ ID NO: 2373) |
| signal peptide and | |
| His tag) | |
| Human TREM2 | DLWFPGESES (SEQ ID NO: 2374) |
| peptide | |
| Human TREM2 | DLWFPGESE (SEQ ID NO: 2375) |
| peptide 9-mer amino | |
| acid sequence | |
| Human TREM2 | DLWFP (SEQ ID NO: 2376) |
| peptide sequence | |
| (residues 140-144 of | |
| full-length TREM2) | |
| Clone CH3C.18.3.4-1 | APELLGGPSVFLFPPKPKDTLMISRTPEVTCVVVDVSHEDPEVKFNWY |
| (CH3C.3.4-1) | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESWGFVWSTYKTTPPVLDSDGSFFLYSKLTVPKSNWQQ |
| | GFVFSCSVMHEALHNHYTQKSLSLSPGK (SEQ ID NO: 2377) |
| Clone CH3C.18.3.4- | APELLGGPSVFLFPPKPKDTLMISRTPEVTCVVVDVSHEDPEVKFNWY |
| 19 (CH3C.3.4-19) | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESWGHVWSTYKTTPPVLDSDGSFFLYSKLTVPKSNWQQ |
| | GYVFSCSVMHEALHNHYTQKSLSLSPGK (SEQ ID NO: 2378) |
| Clone CH3C.18.3.2-3 | APELLGGPSVFLFPPKPKDTLMISRTPEVTCVVVDVSHEDPEVKFNWY |
| (CH3C.3.2-3) | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWVEQKTTPPVLDSDGSFFLYSKLTVPKSTWQQ |
| | GWVFSCSVMHEALHNHYTQKSLSLSPGK (SEQ ID NO: 2379) |
| Clone CH3C.18.3.2- | APELLGGPSVFLFPPKPKDTLMISRTPEVTCVVVDVSHEDPEVKFNWY |
| 14 (CH3C.3.2-14) | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWVGVKTTPPVLDSDGSFFLYSKLTVPKSTWQQ |
| | GWVFSCSVMHEALHNHYTQKSLSLSPGK (SEQ ID NO: 2380) |
| Clone CH3C.18.3.2- | APELLGGPSVFLFPPKPKDTLMISRTPEVTCVVVDVSHEDPEVKFNWY |
| 24 (CH3C.3.2-24) | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWVHTKTTPPVLDSDGSFFLYSKLTVPKSTWQQ |
| | GWVFSCSVMHEALHNHYTQKSLSLSPGK (SEQ ID NO: 2381) |
| Clone CH3C.18.3.4- | APELLGGPSVFLFPPKPKDTLMISRTPEVTCVVVDVSHEDPEVKFNWY |
| 26 (CH3C.3.4-26) | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESWGTVWGTYKTTPPVLDSDGSFFLYSKLTVPKSNWQQ |
| | GYVFSCSVMHEALHNHYTQKSLSLSPGK (SEQ ID NO: 2382) |
| Clone CH3C.18.3.2- | APELLGGPSVFLFPPKPKDTLMISRTPEVTCVVVDVSHEDPEVKFNWY |
| 17 (CH3C.3.2-17) | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESLGHVWVGTKTTPPVLDSDGSFFLYSKLTVPKSTWQQ |
| | GWVFSCSVMHEALHNHYTQKSLSLSPGK (SEQ ID NO: 2383) |
| Clone | APELLGGPSVFLFPPKPKDTLMISRTPEVTCVVVDVSHEDPEVKFNWY |
| CH3C.35.20.1.1 | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESFGTEWSSYKTTPPVLDSDGSFFLYSKLTVSKEEWQQG |
| | FVFSCSVMHEALHNHYTQKSLSLSPGK (SEQ ID NO: 2384) |
| Clone | APELLGGPSVFLFPPKPKDTLMISRTPEVTCVVVDVSHEDPEVKFNWY |
| CH3C.35.23.2.1 | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWANYKTTPPVLDSDGSFFLYSKLTVSKSEWQQ |
| | GFVFSCSVMHEALHNHYTQKSLSLSPGK (SEQ ID NO: 2385) |
| Clone CH3C.35.23.1.1 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESFGTEWSNYKTTPPVLDSDGSFFLYSKLTVSKEEWQQG |
| | FVFSCSVMHEALHNHYTQKSLSLSPGK (SEQ ID NO: 2386) |
| Clone CH3C.35.S413 | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWSSYKTTPPVLDSDGSFFLYSKLTVSKSEWQQG |
| | FVFSCSVMHEALHNHYTQKSLSLSPGK (SEQ ID NO: 2387) |
| Clone | APELLGGPSVFLFPPKPKDTLMISRTPEVTCVVVDVSHEDPEVKFNWY |
| CH3C.35.23.3.1 | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWVNYKTTPPVLDSDGSFFLYSKLTVSKEEWQQ |
| | GFVFSCSVMHEALHNHYTQKSLSLSPGK (SEQ ID NO: 2388) |
| Clone | APELLGGPSVFLFPPKPKDTLMISRTPEVTCVVVDVSHEDPEVKFNWY |
| CH3C.35.N390.1 | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESYGTEWSNYKTTPPVLDSDGSFFLYSKLTVSKSEWQQ |
| | GFVFSCSVMHEALHNHYTQKSLSLSPGK (SEQ ID NO: 2389) |
| Clone | APELLGGPSVFLFPPKPKDTLMISRTPEVTCVVVDVSHEDPEVKFNWY |
| CH3C.35.23.6.1 | VDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGF |
| | YPSDIAVEWESFGTEWVNYKTTPPVLDSDGSFFLYSKLTVSKEEWQQ |
| | GFVFSCSVMHEALHNHYTQKSLSLSPGK (SEQ ID NO: 2390) |
| Clone CH3C.35.21 | APEAAGGPSVFLFPPKPKDTLMISRTPEVTCVVVDVSHEDPEVKFNWY |
| with knob and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLWCLVKG |
| | FYPSDIAVWWESYGTEWSSYKTTPPVLDSDGSFFLYSKLTVTKEEWQ |
| | QGFVFSCSVMHEALHNHYTQKSLSLSPGK (SEQ ID NO: 2391) |
| Clone CH3C.35.21 | APEAAGGPSVFLFPPKPKDTLYITREPEVTCVVVDVSHEDPEVKFNWY |
| with knob, LALAPG, | VDGVEVHNAKTKPREEQYNSTYRVVSVLTVLHQDWLNGKEYKCKV |
| and YTE mutations | SNKALGAPIEKTISKAKGQPREPQVYTLPPSRDELTKNQVSLWCLVKG |
| | FYPSDIAVWWESYGTEWSSYKTTPPVLDSDGSFFLYSKLTVTKEEWQ |
| | QGFVFSCSVMHEALHNHYTQKSLSLSPGK (SEQ ID NO: 2392) |
| Clone CH3C.35.21 | APEAAGGPSVFLFPPKPKDTLMISRTPEVTCVVVDVSHEDPEVKFNWY |
| with hole and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLSCAVKG |
| | FYPSDIAVWWESYGTEWSSYKTTPPVLDSDGSFFLVSKLTVTKEEWQ |
| | QGFVFSCSVMHEALHNHYTQKSLSLSPGK (SEQ ID NO: 2393) |
| Clone CH3C.35.21 | APEAAGGPSVFLFPPKPKDTLYITREPEVTCVVVDVSHEDPEVKFNWY |
| with hole, LALAPG, | VDGVEVHNAKTKPREEQYNSTYRVVSVLTVLHQDWLNGKEYKCKV |
| and YTE mutations | SNKALGAPIEKTISKAKGQPREPQVYTLPPSRDELTKNQVSLSCAVKG |
| | FYPSDIAVWWESYGTEWSSYKTTPPVLDSDGSFFLVSKLTVTKEEWQ |
| | QGFVFSCSVMHEALHNHYTQKSLSLSPGK (SEQ ID NO: 2394) |
| Clone | APELLGGPSVFLFPPKPKDTLMISRTPEVTCVVVDVSHEDPEVKFNWY |
| CH3C.35.20.1.1 with | VDGVEVHNAKTKPREEQYNSTYRVVSVLTVLHQDWLNGKEYKCKV |
| knob mutation | SNKALPAPIEKTISKAKGQPREPQVYTLPPSRDELTKNQVSLWCLVKG |
| | FYPSDIAVEWESFGTEWSSYKTTPPVLDSDGSFFLYSKLTVSKEEWQQ |
| | GFVFSCSVMHEALHNHYTQKSLSLSPGK (SEQ ID NO: 2395) |
| Clone | APEAAGGPSVFLFPPKPKDTLMISRTPEVTCVVVDVSHEDPEVKFNWY |
| CH3C.35.20.1.1 with | VDGVEVHNAKTKPREEQYNSTYRVVSVLTVLHQDWLNGKEYKCKV |
| knob and LALA | SNKALPAPIEKTISKAKGQPREPQVYTLPPSRDELTKNQVSLWCLVKG |
| mutations | FYPSDIAVEWESFGTEWSSYKTTPPVLDSDGSFFLYSKLTVSKEEWQQ |
| | GFVFSCSVMHEALHNHYTQKSLSLSPGK (SEQ ID NO: 2396) |
| Clone | APEAAGGPSVFLFPPKPKDTLMISRTPEVTCVVVDVSHEDPEVKFNWY |
| CH3C.35.20.1.1 with | VDGVEVHNAKTKPREEQYNSTYRVVSVLTVLHQDWLNGKEYKCKV |
| knob and LALAPG | SNKALGAPIEKTISKAKGQPREPQVYTLPPSRDELTKNQVSLWCLVKG |
| mutations | FYPSDIAVEWESFGTEWSSYKTTPPVLDSDGSFFLYSKLTVSKEEWQQ |
| | GFVFSCSVMHEALHNHYTQKSLSLSPGK (SEQ ID NO: 2397) |
| Clone | APELLGGPSVFLFPPKPKDTLYITREPEVTCVVVDVSHEDPEVKFNWY |
| CH3C.35.20.1.1 with | VDGVEVHNAKTKPREEQYNSTYRVVSVLTVLHQDWLNGKEYKCKV |
| knob and YTE | SNKALPAPIEKTISKAKGQPREPQVYTLPPSRDELTKNQVSLWCLVKG |
| mutations | FYPSDIAVEWESFGTEWSSYKTTPPVLDSDGSFFLYSKLTVSKEEWQQ |
| | GFVFSCSVMHEALHNHYTQKSLSLSPGK (SEQ ID NO: 2398) |
| Clone | APEAAGGPSVFLFPPKPKDTLYITREPEVTCVVVDVSHEDPEVKFNWY |
| CH3C.35.20.1.1 with | VDGVEVHNAKTKPREEQYNSTYRVVSVLTVLHQDWLNGKEYKCKV |
| knob, LALA, and | SNKALPAPIEKTISKAKGQPREPQVYTLPPSRDELTKNQVSLWCLVKG |
| YTE mutations | FYPSDIAVEWESFGTEWSSYKTTPPVLDSDGSFFLYSKLTVSKEEWQQ |
| | GFVFSCSVMHEALHNHYTQKSLSLSPGK (SEQ ID NO: 2399) |
| Clone | APEAAGGPSVFLFPPKPKDTLYITREPEVTCVVVDVSHEDPEVKFNWY |
| CH3C.35.20.1.1 with | VDGVEVHNAKTKPREEQYNSTYRVVSVLTVLHQDWLNGKEYKCKV |
| knob, LALAPG, and | SNKALGAPIEKTISKAKGQPREPQVYTLPPSRDELTKNQVSLWCLVKG |
| YTE mutations | FYPSDIAVEWESFGTEWSSYKTTPPVLDSDGSFFLYSKLTVSKEEWQQ |
| | GFVFSCSVMHEALHNHYTQKSLSLSPGK (SEQ ID NO: 2400) |
| Clone | APELLGGPSVFLFPPKPKDTLMISRTPEVTCVVVDVSHEDPEVKFNWY |
| CH3C.35.20.1.1 with | VDGVEVHNAKTKPREEQYNSTYRVVSVLTVLHQDWLNGKEYKCKV |
| hole mutations | SNKALPAPIEKTISKAKGQPREPQVYTLPPSRDELTKNQVSLSCAVKGF |
| | YPSDIAVEWESFGTEWSSYKTTPPVLDSDGSFFLVSKLTVSKEEWQQG |
| | FVFSCSVMHEALHNHYTQKSLSLSPGK (SEQ ID NO: 2401) |
| Clone | APEAAGGPSVFLFPPKPKDTLMISRTPEVTCVVVDVSHEDPEVKFNWY |
| CH3C.35.20.1.1 with | VDGVEVHNAKTKPREEQYNSTYRVVSVLTVLHQDWLNGKEYKCKV |
| hole and LALA | SNKALPAPIEKTISKAKGQPREPQVYTLPPSRDELTKNQVSLSCAVKGF |
| mutations | YPSDIAVEWESFGTEWSSYKTTPPVLDSDGSFFLVSKLTVSKEEWQQG |
| | FVFSCSVMHEALHNHYTQKSLSLSPGK (SEQ ID NO: 2402) |
| Clone | APEAAGGPSVFLFPPKPKDTLMISRTPEVTCVVVDVSHEDPEVKFNWY |
| CH3C.35.20.1.1 with | VDGVEVHNAKTKPREEQYNSTYRVVSVLTVLHQDWLNGKEYKCKV |
| hole and LALAPG | SNKALGAPIEKTISKAKGQPREPQVYTLPPSRDELTKNQVSLSCAVKG |
| mutations | FYPSDIAVEWESFGTEWSSYKTTPPVLDSDGSFFLVSKLTVSKEEWQQ |
| | GFVFSCSVMHEALHNHYTQKSLSLSPGK (SEQ ID NO: 2403) |
| Clone | APELLGGPSVFLFPPKPKDTLYITREPEVTCVVVDVSHEDPEVKFNWY |
| CH3C.35.20.1.1 with | VDGVEVHNAKTKPREEQYNSTYRVVSVLTVLHQDWLNGKEYKCKV |
| hole and YTE | SNKALPAPIEKTISKAKGQPREPQVYTLPPSRDELTKNQVSLSCAVKGF |
| mutations | YPSDIAVEWESFGTEWSSYKTTPPVLDSDGSFFLVSKLTVSKEEWQQG |
| | FVFSCSVMHEALHNHYTQKSLSLSPGK (SEQ ID NO: 2404) |
| Clone | APEAAGGPSVFLFPPKPKDTLYITREPEVTCVVVDVSHEDPEVKFNWY |
| CH3C.35.20.1.1 with | VDGVEVHNAKTKPREEQYNSTYRVVSVLTVLHQDWLNGKEYKCKV |
| hole, LALA, and YTE | SNKALPAPIEKTISKAKGQPREPQVYTLPPSRDELTKNQVSLSCAVKGF |
| mutations | YPSDIAVEWESFGTEWSSYKTTPPVLDSDGSFFLVSKLTVSKEEWQQG |
| | FVFSCSVMHEALHNHYTQKSLSLSPGK (SEQ ID NO: 2405) |
| Clone | APEAAGGPSVFLFPPKPKDTLYITREPEVTCVVVDVSHEDPEVKFNWY |
| CH3C.35.20.1.1 with | VDGVEVHNAKTKPREEQYNSTYRVVSVLTVLHQDWLNGKEYKCKV |
| hole, LALAPG, and | SNKALGAPIEKTISKAKGQPREPQVYTLPPSRDELTKNQVSLSCAVKG |
| YTE mutations | FYPSDIAVEWESFGTEWSSYKTTPPVLDSDGSFFLVSKLTVSKEEWQQ |
| | GFVFSCSVMHEALHNHYTQKSLSLSPGK (SEQ ID NO: 2406) |
| Clone | APELLGGPSVFLFPPKPKDTLMISRTPEVTCVVVDVSHEDPEVKFNWY |
| CH3C.35.23.2.1 with | VDGVEVHNAKTKPREEQYNSTYRVVSVLTVLHQDWLNGKEYKCKV |
| knob mutation | SNKALPAPIEKTISKAKGQPREPQVYTLPPSRDELTKNQVSLWCLVKG |
| | FYPSDIAVEWESYGTEWANYKTTPPVLDSDGSFFLYSKLTVSKSEWQ |
| | QGFVFSCSVMHEALHNHYTQKSLSLSPGK (SEQ ID NO: 2407) |
| Clone | APEAAGGPSVFLFPPKPKDTLMISRTPEVTCVVVDVSHEDPEVKFNWY |
| CH3C.35.23.2.1 with | VDGVEVHNAKTKPREEQYNSTYRVVSVLTVLHQDWLNGKEYKCKV |
| knob and LALA | SNKALPAPIEKTISKAKGQPREPQVYTLPPSRDELTKNQVSLWCLVKG |
| mutations | FYPSDIAVEWESYGTEWANYKTTPPVLDSDGSFFLYSKLTVSKSEWQ |
| | QGFVFSCSVMHEALHNHYTQKSLSLSPGK (SEQ ID NO: 2408) |
| Clone | APEAAGGPSVFLFPPKPKDTLMISRTPEVTCVVVDVSHEDPEVKFNWY |
| CH3C.35.23.2.1 with | VDGVEVHNAKTKPREEQYNSTYRVVSVLTVLHQDWLNGKEYKCKV |
| knob and LALAPG | SNKALGAPIEKTISKAKGQPREPQVYTLPPSRDELTKNQVSLWCLVKG |
| mutations | FYPSDIAVEWESYGTEWANYKTTPPVLDSDGSFFLYSKLTVSKSEWQ |
| | QGFVFSCSVMHEALHNHYTQKSLSLSPGK (SEQ ID NO: 2409) |
| Clone | APELLGGPSVFLFPPKPKDTLYITREPEVTCVVVDVSHEDPEVKFNWY |
| CH3C.35.23.2.1 with | VDGVEVHNAKTKPREEQYNSTYRVVSVLTVLHQDWLNGKEYKCKV |
| knob and YTE | SNKALPAPIEKTISKAKGQPREPQVYTLPPSRDELTKNQVSLWCLVKG |
| mutations | FYPSDIAVEWESYGTEWANYKTTPPVLDSDGSFFLYSKLTVSKSEWQ |
| | QGFVFSCSVMHEALHNHYTQKSLSLSPGK (SEQ ID NO: 2410) |
| Clone | APEAAGGPSVFLFPPKPKDTLYITREPEVTCVVVDVSHEDPEVKFNWY |
| CH3C.35.23.2.1 with | VDGVEVHNAKTKPREEQYNSTYRVVSVLTVLHQDWLNGKEYKCKV |
| knob, LALA, and | SNKALPAPIEKTISKAKGQPREPQVYTLPPSRDELTKNQVSLWCLVKG |
| YTE mutations | FYPSDIAVEWESYGTEWANYKTTPPVLDSDGSFFLYSKLTVSKSEWQ |
| | QGFVFSCSVMHEALHNHYTQKSLSLSPGK (SEQ ID NO: 2411) |
| Clone | APEAAGGPSVFLFPPKPKDTLYITREPEVTCVVVDVSHEDPEVKFNWY |
| CH3C.35.23.2.1 with | VDGVEVHNAKTKPREEQYNSTYRVVSVLTVLHQDWLNGKEYKCKV |
| knob, LALAPG, and | SNKALGAPIEKTISKAKGQPREPQVYTLPPSRDELTKNQVSLWCLVKG |
| YTE mutations | FYPSDIAVEWESYGTEWANYKTTPPVLDSDGSFFLYSKLTVSKSEWQ |
| | QGFVFSCSVMHEALHNHYTQKSLSLSPGK (SEQ ID NO: 2412) |
| Clone | APELLGGPSVFLFPPKPKDTLMISRTPEVTCVVVDVSHEDPEVKFNWY |
| CH3C.35.23.2.1 with | VDGVEVHNAKTKPREEQYNSTYRVVSVLTVLHQDWLNGKEYKCKV |
| hole mutations | SNKALPAPIEKTISKAKGQPREPQVYTLPPSRDELTKNQVSLSCAVKGF |
| | YPSDIAVEWESYGTEWANYKTTPPVLDSDGSFFLVSKLTVSKSEWQQ |
| | GFVFSCSVMHEALHNHYTQKSLSLSPGK (SEQ ID NO: 2413) |
| Clone | APEAAGGPSVFLFPPKPKDTLMISRTPEVTCVVVDVSHEDPEVKFNWY |
| CH3C.35.23.2.1 with | VDGVEVHNAKTKPREEQYNSTYRVVSVLTVLHQDWLNGKEYKCKV |
| hole and LALA | SNKALPAPIEKTISKAKGQPREPQVYTLPPSRDELTKNQVSLSCAVKGF |
| mutations | YPSDIAVEWESYGTEWANYKTTPPVLDSDGSFFLVSKLTVSKSEWQQ |
| | GFVFSCSVMHEALHNHYTQKSLSLSPGK (SEQ ID NO: 2414) |
| Clone | APEAAGGPSVFLFPPKPKDTLMISRTPEVTCVVVDVSHEDPEVKFNWY |
| CH3C.35.23.2.1 with | VDGVEVHNAKTKPREEQYNSTYRVVSVLTVLHQDWLNGKEYKCKV |
| hole and LALAPG | SNKALGAPIEKTISKAKGQPREPQVYTLPPSRDELTKNQVSLSCAVKG |
| mutations | FYPSDIAVEWESYGTEWANYKTTPPVLDSDGSFFLVSKLTVSKSEWQ |
| | QGFVFSCSVMHEALHNHYTQKSLSLSPGK (SEQ ID NO: 2415) |
| Clone | APELLGGPSVFLFPPKPKDTLYITREPEVTCVVVDVSHEDPEVKFNWY |
| CH3C.35.23.2.1 with | VDGVEVHNAKTKPREEQYNSTYRVVSVLTVLHQDWLNGKEYKCKV |
| hole and YTE | SNKALPAPIEKTISKAKGQPREPQVYTLPPSRDELTKNQVSLSCAVKGF |
| mutations | YPSDIAVEWESYGTEWANYKTTPPVLDSDGSFFLVSKLTVSKSEWQQ |
| | GFVFSCSVMHEALHNHYTQKSLSLSPGK (SEQ ID NO: 2416) |
| Clone | APEAAGGPSVFLFPPKPKDTLYITREPEVTCVVVDVSHEDPEVKFNWY |
| CH3C.35.23.2.1 with | VDGVEVHNAKTKPREEQYNSTYRVVSVLTVLHQDWLNGKEYKCKV |
| hole, LALA, and YTE | SNKALPAPIEKTISKAKGQPREPQVYTLPPSRDELTKNQVSLSCAVKGF |
| mutations | YPSDIAVEWESYGTEWANYKTTPPVLDSDGSFFLVSKLTVSKSEWQQ |
| | GFVFSCSVMHEALHNHYTQKSLSLSPGK (SEQ ID NO: 2417) |
| Clone | APEAAGGPSVFLFPPKPKDTLYITREPEVTCVVVDVSHEDPEVKFNWY |
| CH3C.35.23.2.1 with | VDGVEVHNAKTKPREEQYNSTYRVVSVLTVLHQDWLNGKEYKCKV |
| hole, LALAPG, and | SNKALGAPIEKTISKAKGQPREPQVYTLPPSRDELTKNQVSLSCAVKG |
| YTE mutations | FYPSDIAVEWESYGTEWANYKTTPPVLDSDGSFFLVSKLTVSKSEWQ |
| | QGFVFSCSVMHEALHNHYTQKSLSLSPGK (SEQ ID NO: 2418) |
| Clone CH3C.35.23.1.1 | APELLGGPSVFLFPPKPKDTLMISRTPEVTCVVVDVSHEDPEVKFNWY |
| with knob | VDGVEVHNAKTKPREEQYNSTYRVVSVLTVLHQDWLNGKEYKCKV |
| mutation | SNKALPAPIEKTISKAKGQPREPQVYTLPPSRDELTKNQVSLWCLVKG |
| | FYPSDIAVEWESFGTEWSNYKTTPPVLDSDGSFFLYSKLTVSKEEWQQ |
| | GFVFSCSVMHEALHNHYTQKSLSLSPGK (SEQ ID NO: 2419) |
| Clone CH3C.35.23.1.1 | APEAAGGPSVFLFPPKPKDTLMISRTPEVTCVVVDVSHEDPEVKFNWY |
| with knob and | VDGVEVHNAKTKPREEQYNSTYRVVSVLTVLHQDWLNGKEYKCKV |
| LALA mutations | SNKALPAPIEKTISKAKGQPREPQVYTLPPSRDELTKNQVSLWCLVKG |
| | FYPSDIAVEWESFGTEWSNYKTTPPVLDSDGSFFLYSKLTVSKEEWQQ |
| | GFVFSCSVMHEALHNHYTQKSLSLSPGK (SEQ ID NO: 2420) |
| Clone CH3C.35.23.1.1 | APEAAGGPSVFLFPPKPKDTLMISRTPEVTCVVVDVSHEDPEVKFNWY |
| with knob and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLWCLVKG |
| | FYPSDIAVEWESFGTEWSNYKTTPPVLDSDGSFFLYSKLTVSKEEWQQ |
| | GFVFSCSVMHEALHNHYTQKSLSLSPGK (SEQ ID NO: 2421) |
| Clone CH3C.35.23.1.1 | APELLGGPSVFLFPPKPKDTLYITREPEVTCVVVDVSHEDPEVKFNWY |
| with knob and | VDGVEVHNAKTKPREEQYNSTYRVVSVLTVLHQDWLNGKEYKCKV |
| YTE mutations | SNKALPAPIEKTISKAKGQPREPQVYTLPPSRDELTKNQVSLWCLVKG |
| | FYPSDIAVEWESFGTEWSNYKTTPPVLDSDGSFFLYSKLTVSKEEWQQ |
| | GFVFSCSVMHEALHNHYTQKSLSLSPGK (SEQ ID NO: 2422) |
| Clone CH3C.35.23.1.1 | APEAAGGPSVFLFPPKPKDTLYITREPEVTCVVVDVSHEDPEVKFNWY |
| with knob, LALA, | VDGVEVHNAKTKPREEQYNSTYRVVSVLTVLHQDWLNGKEYKCKV |
| and YTE mutations | SNKALPAPIEKTISKAKGQPREPQVYTLPPSRDELTKNQVSLWCLVKG |
| | FYPSDIAVEWESFGTEWSNYKTTPPVLDSDGSFFLYSKLTVSKEEWQQ |
| | GFVFSCSVMHEALHNHYTQKSLSLSPGK (SEQ ID NO: 2423) |
| Clone CH3C.35.23.1.1 | APEAAGGPSVFLFPPKPKDTLYITREPEVTCVVVDVSHEDPEVKFNWY |
| with knob, | VDGVEVHNAKTKPREEQYNSTYRVVSVLTVLHQDWLNGKEYKCKV |
| LALAPG, and YTE | SNKALGAPIEKTISKAKGQPREPQVYTLPPSRDELTKNQVSLWCLVKG |
| mutations | FYPSDIAVEWESFGTEWSNYKTTPPVLDSDGSFFLYSKLTVSKEEWQQ |
| | GFVFSCSVMHEALHNHYTQKSLSLSPGK (SEQ ID NO: 2424) |
| Clone CH3C.35.23.1.1 | APELLGGPSVFLFPPKPKDTLMISRTPEVTCVVVDVSHEDPEVKFNWY |
| with hole | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLSCAVKGF |
| | YPSDIAVEWESFGTEWSNYKTTPPVLDSDGSFFLVSKLTVSKEEWQQG |
| | FVFSCSVMHEALHNHYTQKSLSLSPGK (SEQ ID NO: 2425) |
| Clone CH3C.35.23.1.1 | APEAAGGPSVFLFPPKPKDTLMISRTPEVTCVVVDVSHEDPEVKFNWY |
| with hole and | VDGVEVHNAKTKPREEQYNSTYRVVSVLTVLHQDWLNGKEYKCKV |
| LALA mutations | SNKALPAPIEKTISKAKGQPREPQVYTLPPSRDELTKNQVSLSCAVKGF |
| | YPSDIAVEWESFGTEWSNYKTTPPVLDSDGSFFLVSKLTVSKEEWQQG |
| | FVFSCSVMHEALHNHYTQKSLSLSPGK (SEQ ID NO: 2426) |
| Clone CH3C.35.23.1.1 | APEAAGGPSVFLFPPKPKDTLMISRTPEVTCVVVDVSHEDPEVKFNWY |
| with hole and | VDGVEVHNAKTKPREEQYNSTYRVVSVLTVLHQDWLNGKEYKCKV |
| LALAPG mutations | SNKALGAPIEKTISKAKGQPREPQVYTLPPSRDELTKNQVSLSCAVKG |
| | FYPSDIAVEWESFGTEWSNYKTTPPVLDSDGSFFLVSKLTVSKEEWQQ |
| | GFVFSCSVMHEALHNHYTQKSLSLSPGK (SEQ ID NO: 2427) |
| Clone CH3C.35.23.1.1 | APELLGGPSVFLFPPKPKDTLYITREPEVTCVVVDVSHEDPEVKFNWY |
| with hole and | VDGVEVHNAKTKPREEQYNSTYRVVSVLTVLHQDWLNGKEYKCKV |
| YTE mutations | SNKALPAPIEKTISKAKGQPREPQVYTLPPSRDELTKNQVSLSCAVKGF |
| | YPSDIAVEWESFGTEWSNYKTTPPVLDSDGSFFLVSKLTVSKEEWQQG |
| | FVFSCSVMHEALHNHYTQKSLSLSPGK (SEQ ID NO: 2428) |
| Clone CH3C.35.23.1.1 | APEAAGGPSVFLFPPKPKDTLYITREPEVTCVVVDVSHEDPEVKFNWY |
| with hole, LALA, | VDGVEVHNAKTKPREEQYNSTYRVVSVLTVLHQDWLNGKEYKCKV |
| and YTE mutations | SNKALPAPIEKTISKAKGQPREPQVYTLPPSRDELTKNQVSLSCAVKGF |
| | YPSDIAVEWESFGTEWSNYKTTPPVLDSDGSFFLVSKLTVSKEEWQQG |
| | FVFSCSVMHEALHNHYTQKSLSLSPGK (SEQ ID NO: 2429) |
| Clone CH3C.35.23.1.1 | APEAAGGPSVFLFPPKPKDTLYITREPEVTCVVVDVSHEDPEVKFNWY |
| with hole, | VDGVEVHNAKTKPREEQYNSTYRVVSVLTVLHQDWLNGKEYKCKV |
| LALAPG, and YTE | SNKALGAPIEKTISKAKGQPREPQVYTLPPSRDELTKNQVSLSCAVKG |
| mutations | FYPSDIAVEWESFGTEWSNYKTTPPVLDSDGSFFLVSKLTVSKEEWQQ |
| | GFVFSCSVMHEALHNHYTQKSLSLSPGK (SEQ ID NO: 2430) |
| Clone CH3C.35.20.1 | APELLGGPSVFLFPPKPKDTLMISRTPEVTCVVVDVSHEDPEVKFNWY |
| M428L and N434S | VDGVEVHNAKTKPREEQYNSTYRVVSVLTVLHQDWLNGKEYKCKV |
| mutations | SNKALPAPIEKTISKAKGQPREPQVYTLPPSRDELTKNQVSLTCLVKGF |
| | YPSDIAVEWESFGTEWSSYKTTPPVLDSDGSFFLYSKLTVTKEEWQQG |
| | FVFSCSVLHEALHSHYTQKSLSLSPGK (SEQ ID NO: 2431) |
| Clone CH3C.35.20.1 | APELLGGPSVFLFPPKPKDTLMISRTPEVTCVVVDVSHEDPEVKFNWY |
| with knob and M428L | VDGVEVHNAKTKPREEQYNSTYRVVSVLTVLHQDWLNGKEYKCKV |
| and N4345 mutations | SNKALPAPIEKTISKAKGQPREPQVYTLPPSRDELTKNQVSLWCLVKG |
| | FYPSDIAVEWESFGTEWSSYKTTPPVLDSDGSFFLYSKLTVTKEEWQQ |
| | GFVFSCSVLHEALHSHYTQKSLSLSPGK (SEQ ID NO: 2432) |
| Clone CH3C.35.20.1 | APEAAGGPSVFLFPPKPKDTLMISRTPEVTCVVVDVSHEDPEVKFNWY |
| with knob, LALA, | VDGVEVHNAKTKPREEQYNSTYRVVSVLTVLHQDWLNGKEYKCKV |
| and M428L and | SNKALPAPIEKTISKAKGQPREPQVYTLPPSRDELTKNQVSLWCLVKG |
| N434S mutations | FYPSDIAVEWESFGTEWSSYKTTPPVLDSDGSFFLYSKLTVTKEEWQQ |
| | GFVFSCSVLHEALHSHYTQKSLSLSPGK (SEQ ID NO: 2433) |
| Clone CH3C.35.20.1 | APEAAGGPSVFLFPPKPKDTLMISRTPEVTCVVVDVSHEDPEVKFNWY |
| with knob, LALAPG, | VDGVEVHNAKTKPREEQYNSTYRVVSVLTVLHQDWLNGKEYKCKV |
| and M428L and | SNKALGAPIEKTISKAKGQPREPQVYTLPPSRDELTKNQVSLWCLVKG |
| N434S mutations | FYPSDIAVEWESFGTEWSSYKTTPPVLDSDGSFFLYSKLTVTKEEWQQ |
| | GFVFSCSVLHEALHSHYTQKSLSLSPGK (SEQ ID NO: 2434) |
| Clone CH3C.35.20.1 | APELLGGPSVFLFPPKPKDTLMISRTPEVTCVVVDVSHEDPEVKFNWY |
| with hole and M428L | VDGVEVHNAKTKPREEQYNSTYRVVSVLTVLHQDWLNGKEYKCKV |
| and N4345 mutations | SNKALPAPIEKTISKAKGQPREPQVYTLPPSRDELTKNQVSLSCAVKGF |
| | YPSDIAVEWESFGTEWSSYKTTPPVLDSDGSFFLVSKLTVTKEEWQQG |
| | FVFSCSVLHEALHSHYTQKSLSLSPGK (SEQ ID NO: 2435) |
| Clone CH3C.35.20.1 | APEAAGGPSVFLFPPKPKDTLMISRTPEVTCVVVDVSHEDPEVKFNWY |
| with hole, LALA, and | VDGVEVHNAKTKPREEQYNSTYRVVSVLTVLHQDWLNGKEYKCKV |
| M428L and N434S | SNKALPAPIEKTISKAKGQPREPQVYTLPPSRDELTKNQVSLSCAVKGF |
| mutations | YPSDIAVEWESFGTEWSSYKTTPPVLDSDGSFFLVSKLTVTKEEWQQG |
| | FVFSCSVLHEALHSHYTQKSLSLSPGK (SEQ ID NO: 2436) |
| Clone CH3C.35.20.1 | APEAAGGPSVFLFPPKPKDTLMISRTPEVTCVVVDVSHEDPEVKFNWY |
| with hole, LALAPG, | VDGVEVHNAKTKPREEQYNSTYRVVSVLTVLHQDWLNGKEYKCKV |
| and M428L and | SNKALGAPIEKTISKAKGQPREPQVYTLPPSRDELTKNQVSLSCAVKG |
| N434S mutations | FYPSDIAVEWESFGTEWSSYKTTPPVLDSDGSFFLVSKLTVTKEEWQQ |
| | GFVFSCSVLHEALHSHYTQKSLSLSPGK (SEQ ID NO: 2437) |
| Clone | APELLGGPSVFLFPPKPKDTLMISRTPEVTCVVVDVSHEDPEVKFNWY |
| CH3C.35.20.1.1 with | VDGVEVHNAKTKPREEQYNSTYRVVSVLTVLHQDWLNGKEYKCKV |
| M428L and N4345 | SNKALPAPIEKTISKAKGQPREPQVYTLPPSRDELTKNQVSLTCLVKGF |
| mutations | YPSDIAVEWESFGTEWSSYKTTPPVLDSDGSFFLYSKLTVSKEEWQQG |
| | FVFSCSVLHEALHSHYTQKSLSLSPGK (SEQ ID NO: 2438) |
| Clone | APELLGGPSVFLFPPKPKDTLMISRTPEVTCVVVDVSHEDPEVKFNWY |
| CH3C.35.20.1.1 with | VDGVEVHNAKTKPREEQYNSTYRVVSVLTVLHQDWLNGKEYKCKV |
| knob and M428L and | SNKALPAPIEKTISKAKGQPREPQVYTLPPSRDELTKNQVSLWCLVKG |
| N434S mutations | FYPSDIAVEWESFGTEWSSYKTTPPVLDSDGSFFLYSKLTVSKEEWQQ |
| | GFVFSCSVLHEALHSHYTQKSLSLSPGK (SEQ ID NO: 2439) |
| Clone | APEAAGGPSVFLFPPKPKDTLMISRTPEVTCVVVDVSHEDPEVKFNWY |
| CH3C.35.20.1.1 with | VDGVEVHNAKTKPREEQYNSTYRVVSVLTVLHQDWLNGKEYKCKV |
| knob, LALA, and | SNKALPAPIEKTISKAKGQPREPQVYTLPPSRDELTKNQVSLWCLVKG |
| M428L and N434S | FYPSDIAVEWESFGTEWSSYKTTPPVLDSDGSFFLYSKLTVSKEEWQQ |
| mutations | GFVFSCSVLHEALHSHYTQKSLSLSPGK (SEQ ID NO: 2440) |
| Clone | APEAAGGPSVFLFPPKPKDTLMISRTPEVTCVVVDVSHEDPEVKFNWY |
| CH3C.35.20.1.1 with | VDGVEVHNAKTKPREEQYNSTYRVVSVLTVLHQDWLNGKEYKCKV |
| knob, LALAPG, and | SNKALGAPIEKTISKAKGQPREPQVYTLPPSRDELTKNQVSLWCLVKG |
| M428L and N434S | FYPSDIAVEWESFGTEWSSYKTTPPVLDSDGSFFLYSKLTVSKEEWQQ |
| mutations | GFVFSCSVLHEALHSHYTQKSLSLSPGK (SEQ ID NO: 2441) |
| APELLGGP | SVFLFPPKPKDTLMISRTPEVTCVVVDVSHEDPEVKFNWY |
| Clone | VDGVEVHNAKTKPREEQYNSTYRVVSVLTVLHQDWLNGKEYKCKV |
| CH3C.35.20.1.1 with | SNKALPAPIEKTISKAKGQPREPQVYTLPPSRDELTKNQVSLSCAVKGF |
| hole and M428L and | YPSDIAVEWESFGTEWSSYKTTPPVLDSDGSFFLVSKLTVSKEEWQQG |
| N434S mutations | FVFSCSVLHEALHSHYTQKSLSLSPGK (SEQ ID NO: 2442) |
| Clone | APEAAGGPSVFLFPPKPKDTLMISRTPEVTCVVVDVSHEDPEVKFNWY |
| CH3C.35.20.1.1 with | VDGVEVHNAKTKPREEQYNSTYRVVSVLTVLHQDWLNGKEYKCKV |
| hole, LALA, and | SNKALPAPIEKTISKAKGQPREPQVYTLPPSRDELTKNQVSLSCAVKGF |
| M428L and N434S | YPSDIAVEWESFGTEWSSYKTTPPVLDSDGSFFLVSKLTVSKEEWQQG |
| mutations | FVFSCSVLHEALHSHYTQKSLSLSPGK (SEQ ID NO: 2443) |
| Clone | APEAAGGPSVFLFPPKPKDTLMISRTPEVTCVVVDVSHEDPEVKFNWY |
| CH3C.35.20.1.1 with | VDGVEVHNAKTKPREEQYNSTYRVVSVLTVLHQDWLNGKEYKCKV |
| hole, LALAPG, and | SNKALGAPIEKTISKAKGQPREPQVYTLPPSRDELTKNQVSLSCAVKG |
| M428L and N434S | FYPSDIAVEWESFGTEWSSYKTTPPVLDSDGSFFLVSKLTVSKEEWQQ |
| mutations | GFVFSCSVLHEALHSHYTQKSLSLSPGK (SEQ ID NO: 2444) |
| Clone CH3C.35.21 | APELLGGPSVFLFPPKPKDTLMISRTPEVTCVVVDVSHEDPEVKFNWY |
| with M428L and | VDGVEVHNAKTKPREEQYNSTYRVVSVLTVLHQDWLNGKEYKCKV |
| N434S mutations | SNKALPAPIEKTISKAKGQPREPQVYTLPPSRDELTKNQVSLTCLVKGF |
| | YPSDIAVWWESYGTEWSSYKTTPPVLDSDGSFFLYSKLTVTKEEWQQ |
| | GFVFSCSVLHEALHSHYTQKSLSLSPGK (SEQ ID NO: 2445) |
| Clone CH3C.35.21 | APELLGGPSVFLFPPKPKDTLMISRTPEVTCVVVDVSHEDPEVKFNWY |
| with knob and M428L | VDGVEVHNAKTKPREEQYNSTYRVVSVLTVLHQDWLNGKEYKCKV |
| and N4345 mutations | SNKALPAPIEKTISKAKGQPREPQVYTLPPSRDELTKNQVSLWCLVKG |
| | FYPSDIAVWWESYGTEWSSYKTTPPVLDSDGSFFLYSKLTVTKEEWQ |
| | QGFVFSCSVLHEALHSHYTQKSLSLSPGK (SEQ ID NO: 2446) |
| Clone CH3C.35.21 | APEAAGGPSVFLFPPKPKDTLMISRTPEVTCVVVDVSHEDPEVKFNWY |
| with knob, LALA, | VDGVEVHNAKTKPREEQYNSTYRVVSVLTVLHQDWLNGKEYKCKV |
| and M428L and | SNKALPAPIEKTISKAKGQPREPQVYTLPPSRDELTKNQVSLWCLVKG |
| N434S mutations | FYPSDIAVWWESYGTEWSSYKTTPPVLDSDGSFFLYSKLTVTKEEWQ |
| | QGFVFSCSVLHEALHSHYTQKSLSLSPGK (SEQ ID NO: 2447) |
| Clone CH3C.35.21 | APEAAGGPSVFLFPPKPKDTLMISRTPEVTCVVVDVSHEDPEVKFNWY |
| with knob, LALAPG, | VDGVEVHNAKTKPREEQYNSTYRVVSVLTVLHQDWLNGKEYKCKV |
| and M428L and | SNKALGAPIEKTISKAKGQPREPQVYTLPPSRDELTKNQVSLWCLVKG |
| N434S mutations | FYPSDIAVWWESYGTEWSSYKTTPPVLDSDGSFFLYSKLTVTKEEWQ |
| | QGFVFSCSVLHEALHSHYTQKSLSLSPGK (SEQ ID NO: 2448) |
| Clone CH3C.35.21 | APELLGGPSVFLFPPKPKDTLMISRTPEVTCVVVDVSHEDPEVKFNWY |
| with hole and M428L | VDGVEVHNAKTKPREEQYNSTYRVVSVLTVLHQDWLNGKEYKCKV |
| and N4345 mutations | SNKALPAPIEKTISKAKGQPREPQVYTLPPSRDELTKNQVSLSCAVKGF |
| | YPSDIAVWWESYGTEWSSYKTTPPVLDSDGSFFLVSKLTVTKEEWQQ |
| | GFVFSCSVLHEALHSHYTQKSLSLSPGK (SEQ ID NO: 2449) |
| Clone CH3C.35.21 | APEAAGGPSVFLFPPKPKDTLMISRTPEVTCVVVDVSHEDPEVKFNWY |
| with hole. LALA, and | VDGVEVHNAKTKPREEQYNSTYRVVSVLTVLHQDWLNGKEYKCKV |
| M428L and N4345 | SNKALPAPIEKTISKAKGQPREPQVYTLPPSRDELTKNQVSLSCAVKGF |
| mutations | YPSDIAVWWESYGTEWSSYKTTPPVLDSDGSFFLVSKLTVTKEEWQQ |
| | GFVFSCSVLHEALHSHYTQKSLSLSPGK (SEQ ID NO: 2450) |
| Clone CH3C.35.21 | APEAAGGPSVFLFPPKPKDTLMISRTPEVTCVVVDVSHEDPEVKFNWY |
| with hole, LALAPG, | VDGVEVHNAKTKPREEQYNSTYRVVSVLTVLHQDWLNGKEYKCKV |
| and M428L and | SNKALGAPIEKTISKAKGQPREPQVYTLPPSRDELTKNQVSLSCAVKG |
| N434S mutations | FYPSDIAVWWESYGTEWSSYKTTPPVLDSDGSFFLVSKLTVTKEEWQ |
| | QGFVFSCSVLHEALHSHYTQKSLSLSPGK (SEQ ID NO: 2451) |
| Clone CH3C.35.21.17.2 | APELLGGPSVFLFPPKPKDTLMISRTPEVTCVVVDVSHEDPEVKFNWY |
| with M428L and | VDGVEVHNAKTKPREEQYNSTYRVVSVLTVLHQDWLNGKEYKCKV |
| N434S mutations | SNKALPAPIEKTISKAKGQPREPQVYTLPPSRDELTKNQVSLTCLVKGF |
| | YPSDIAVLWESYGTEWASYKTTPPVLDSDGSFFLYSKLTVTKEEWQQ |
| | GFVFSCSVLHEALHSHYTQKSLSLSPGK (SEQ ID NO: 2452) |
| Clone CH3C.35.21.17.2 | APELLGGPSVFLFPPKPKDTLMISRTPEVTCVVVDVSHEDPEVKFNWY |
| with knob and | VDGVEVHNAKTKPREEQYNSTYRVVSVLTVLHQDWLNGKEYKCKV |
| M428L and N434S | SNKALPAPIEKTISKAKGQPREPQVYTLPPSRDELTKNQVSLWCLVKG |
| mutations | FYPSDIAVLWESYGTEWASYKTTPPVLDSDGSFFLYSKLTVTKEEWQ |
| | QGFVFSCSVLHEALHSHYTQKSLSLSPGK (SEQ ID NO: 2453) |
| Clone CH3C.35.21.17.2 | APEAAGGPSVFLFPPKPKDTLMISRTPEVTCVVVDVSHEDPEVKFNWY |
| with knob, | VDGVEVHNAKTKPREEQYNSTYRVVSVLTVLHQDWLNGKEYKCKV |
| LALA, and M428L | SNKALPAPIEKTISKAKGQPREPQVYTLPPSRDELTKNQVSLWCLVKG |
| and N4345 mutations | FYPSDIAVLWESYGTEWASYKTTPPVLDSDGSFFLYSKLTVTKEEWQ |
| | QGFVFSCSVLHEALHSHYTQKSLSLSPGK (SEQ ID NO: 2454) |
| Clone CH3C.35.21.17.2 | APEAAGGPSVFLFPPKPKDTLMISRTPEVTCVVVDVSHEDPEVKFNWY |
| with knob, | VDGVEVHNAKTKPREEQYNSTYRVVSVLTVLHQDWLNGKEYKCKV |
| LALAPG, and | SNKALGAPIEKTISKAKGQPREPQVYTLPPSRDELTKNQVSLWCLVKG |
| M428L and N434S | FYPSDIAVLWESYGTEWASYKTTPPVLDSDGSFFLYSKLTVTKEEWQ |
| mutations | QGFVFSCSVLHEALHSHYTQKSLSLSPGK (SEQ ID NO: 2455) |
| Clone CH3C.35.21.17.2 | APELLGGPSVFLFPPKPKDTLMISRTPEVTCVVVDVSHEDPEVKFNWY |
| with hole and | VDGVEVHNAKTKPREEQYNSTYRVVSVLTVLHQDWLNGKEYKCKV |
| M428L and N4345 | SNKALPAPIEKTISKAKGQPREPQVYTLPPSRDELTKNQVSLSCAVKGF |
| mutations | YPSDIAVLWESYGTEWASYKTTPPVLDSDGSFFLVSKLTVTKEEWQQ |
| | GFVFSCSVLHEALHSHYTQKSLSLSPGK (SEQ ID NO: 2456) |
| Clone CH3C.35.21.17.2 | APEAAGGPSVFLFPPKPKDTLMISRTPEVTCVVVDVSHEDPEVKFNWY |
| with hole, | VDGVEVHNAKTKPREEQYNSTYRVVSVLTVLHQDWLNGKEYKCKV |
| LALA, and M428L | SNKALPAPIEKTISKAKGQPREPQVYTLPPSRDELTKNQVSLSCAVKGF |
| and N4345 mutations | YPSDIAVLWESYGTEWASYKTTPPVLDSDGSFFLVSKLTVTKEEWQQ |
| | GFVFSCSVLHEALHSHYTQKSLSLSPGK (SEQ ID NO: 2457) |
| Clone CH3C.35.21.17.2 | APEAAGGPSVFLFPPKPKDTLMISRTPEVTCVVVDVSHEDPEVKFNWY |
| with hole, | VDGVEVHNAKTKPREEQYNSTYRVVSVLTVLHQDWLNGKEYKCKV |
| LALAPG, and | SNKALGAPIEKTISKAKGQPREPQVYTLPPSRDELTKNQVSLSCAVKG |
| M428L and N434S | FYPSDIAVLWESYGTEWASYKTTPPVLDSDGSFFLVSKLTVTKEEWQ |
| mutations | QGFVFSCSVLHEALHSHYTQKSLSLSPGK (SEQ ID NO: 2458) |
| Clone CH3C.35.23 | APELLGGPSVFLFPPKPKDTLMISRTPEVTCVVVDVSHEDPEVKFNWY |
| with M428L and | VDGVEVHNAKTKPREEQYNSTYRVVSVLTVLHQDWLNGKEYKCKV |
| N434S mutations | SNKALPAPIEKTISKAKGQPREPQVYTLPPSRDELTKNQVSLTCLVKGF |
| | YPSDIAVEWESYGTEWSNYKTTPPVLDSDGSFFLYSKLTVTKEEWQQ |
| | GFVFSCSVLHEALHSHYTQKSLSLSPGK (SEQ ID NO: 2459) |
| Clone CH3C.35.23 | APELLGGPSVFLFPPKPKDTLMISRTPEVTCVVVDVSHEDPEVKFNWY |
| with knob and M428L | VDGVEVHNAKTKPREEQYNSTYRVVSVLTVLHQDWLNGKEYKCKV |
| and N434S mutations | SNKALPAPIEKTISKAKGQPREPQVYTLPPSRDELTKNQVSLWCLVKG |
| | FYPSDIAVEWESYGTEWSNYKTTPPVLDSDGSFFLYSKLTVTKEEWQ |
| | QGFVFSCSVLHEALHSHYTQKSLSLSPGK (SEQ ID NO: 2460) |
| Clone CH3C.35.23 | APEAAGGPSVFLFPPKPKDTLMISRTPEVTCVVVDVSHEDPEVKFNWY |
| with knob, LALA, | VDGVEVHNAKTKPREEQYNSTYRVVSVLTVLHQDWLNGKEYKCKV |
| and M428L and | SNKALPAPIEKTISKAKGQPREPQVYTLPPSRDELTKNQVSLWCLVKG |
| N434S mutations | FYPSDIAVEWESYGTEWSNYKTTPPVLDSDGSFFLYSKLTVTKEEWQ |
| | QGFVFSCSVLHEALHSHYTQKSLSLSPGK (SEQ ID NO: 2461) |
| Clone CH3C.35.23 | APEAAGGPSVFLFPPKPKDTLMISRTPEVTCVVVDVSHEDPEVKFNWY |
| with knob, LALAPG, | VDGVEVHNAKTKPREEQYNSTYRVVSVLTVLHQDWLNGKEYKCKV |
| and M428L and | SNKALGAPIEKTISKAKGQPREPQVYTLPPSRDELTKNQVSLWCLVKG |
| N434S mutations | FYPSDIAVEWESYGTEWSNYKTTPPVLDSDGSFFLYSKLTVTKEEWQ |
| | QGFVFSCSVLHEALHSHYTQKSLSLSPGK (SEQ ID NO: 2462) |
| Clone CH3C.35.23 | APELLGGPSVFLFPPKPKDTLMISRTPEVTCVVVDVSHEDPEVKFNWY |
| with hole and M428L | VDGVEVHNAKTKPREEQYNSTYRVVSVLTVLHQDWLNGKEYKCKV |
| and N4345 mutations | SNKALPAPIEKTISKAKGQPREPQVYTLPPSRDELTKNQVSLSCAVKGF |
| | YPSDIAVEWESYGTEWSNYKTTPPVLDSDGSFFLVSKLTVTKEEWQQ |
| | GFVFSCSVLHEALHSHYTQKSLSLSPGK (SEQ ID NO: 2463) |
| Clone CH3C.35.23 | APEAAGGPSVFLFPPKPKDTLMISRTPEVTCVVVDVSHEDPEVKFNWY |
| with hole. LALA, and | VDGVEVHNAKTKPREEQYNSTYRVVSVLTVLHQDWLNGKEYKCKV |
| M428L and N4345 | SNKALPAPIEKTISKAKGQPREPQVYTLPPSRDELTKNQVSLSCAVKGF |
| mutations | YPSDIAVEWESYGTEWSNYKTTPPVLDSDGSFFLVSKLTVTKEEWQQ |
| | GFVFSCSVLHEALHSHYTQKSLSLSPGK (SEQ ID NO: 2464) |
| Clone CH3C.35.23 | APEAAGGPSVFLFPPKPKDTLMISRTPEVTCVVVDVSHEDPEVKFNWY |
| with hole, LALAPG, | VDGVEVHNAKTKPREEQYNSTYRVVSVLTVLHQDWLNGKEYKCKV |
| and M428L and | SNKALGAPIEKTISKAKGQPREPQVYTLPPSRDELTKNQVSLSCAVKG |
| N434S mutations | FYPSDIAVEWESYGTEWSNYKTTPPVLDSDGSFFLVSKLTVTKEEWQ |
| | QGFVFSCSVLHEALHSHYTQKSLSLSPGK (SEQ ID NO: 2465) |
| Clone CH3C.35.23.1.1 | APELLGGPSVFLFPPKPKDTLMISRTPEVTCVVVDVSHEDPEVKFNWY |
| with M428L and | VDGVEVHNAKTKPREEQYNSTYRVVSVLTVLHQDWLNGKEYKCKV |
| N434S mutations | SNKALPAPIEKTISKAKGQPREPQVYTLPPSRDELTKNQVSLTCLVKGF |
| | YPSDIAVEWESFGTEWSNYKTTPPVLDSDGSFFLYSKLTVSKEEWQQG |
| | FVFSCSVLHEALHSHYTQKSLSLSPGK (SEQ ID NO: 2466) |
| Clone CH3C.35.23.1.1 | APELLGGPSVFLFPPKPKDTLMISRTPEVTCVVVDVSHEDPEVKFNWY |
| with knob and | VDGVEVHNAKTKPREEQYNSTYRVVSVLTVLHQDWLNGKEYKCKV |
| M428L and N4345 | SNKALPAPIEKTISKAKGQPREPQVYTLPPSRDELTKNQVSLWCLVKG |
| mutations | FYPSDIAVEWESFGTEWSNYKTTPPVLDSDGSFFLYSKLTVSKEEWQQ |
| | GFVFSCSVLHEALHSHYTQKSLSLSPGK (SEQ ID NO: 2467) |
| Clone CH3C.35.23.1.1 | APEAAGGPSVFLFPPKPKDTLMISRTPEVTCVVVDVSHEDPEVKFNWY |
| with knob, LALA, | VDGVEVHNAKTKPREEQYNSTYRVVSVLTVLHQDWLNGKEYKCKV |
| and M428L and | SNKALPAPIEKTISKAKGQPREPQVYTLPPSRDELTKNQVSLWCLVKG |
| N434S mutations | FYPSDIAVEWESFGTEWSNYKTTPPVLDSDGSFFLYSKLTVSKEEWQQ |
| | GFVFSCSVLHEALHSHYTQKSLSLSPGK (SEQ ID NO: 2468) |
| Clone CH3C.35.23.1.1 | APEAAGGPSVFLFPPKPKDTLMISRTPEVTCVVVDVSHEDPEVKFNWY |
| with knob, | VDGVEVHNAKTKPREEQYNSTYRVVSVLTVLHQDWLNGKEYKCKV |
| LALAPG, and | SNKALGAPIEKTISKAKGQPREPQVYTLPPSRDELTKNQVSLWCLVKG |
| M428L and N434S | FYPSDIAVEWESFGTEWSNYKTTPPVLDSDGSFFLYSKLTVSKEEWQQ |
| mutations | GFVFSCSVLHEALHSHYTQKSLSLSPGK (SEQ ID NO: 2469) |
| | APELLGGPSVFLFPPKPKDTLMISRTPEVTCVVVDVSHEDPEVKFNWY |
| Clone CH3C.35.23.1.1 | VDGVEVHNAKTKPREEQYNSTYRVVSVLTVLHQDWLNGKEYKCKV |
| with hole and | SNKALPAPIEKTISKAKGQPREPQVYTLPPSRDELTKNQVSLSCAVKGF |
| M428L and N434S | YPSDIAVEWESFGTEWSNYKTTPPVLDSDGSFFLVSKLTVSKEEWQQG |
| mutations | FVFSCSVLHEALHSHYTQKSLSLSPGK (SEQ ID NO: 2470) |
| Clone CH3C.35.23.1.1 | APEAAGGPSVFLFPPKPKDTLMISRTPEVTCVVVDVSHEDPEVKFNWY |
| with hole, LALA, | VDGVEVHNAKTKPREEQYNSTYRVVSVLTVLHQDWLNGKEYKCKV |
| and M428L and | SNKALPAPIEKTISKAKGQPREPQVYTLPPSRDELTKNQVSLSCAVKGF |
| N434S mutations | YPSDIAVEWESFGTEWSNYKTTPPVLDSDGSFFLVSKLTVSKEEWQQG |
| | FVFSCSVLHEALHSHYTQKSLSLSPGK (SEQ ID NO: 2471) |
| Clone CH3C.35.23.1.1 | APEAAGGPSVFLFPPKPKDTLMISRTPEVTCVVVDVSHEDPEVKFNWY |
| with hole, | VDGVEVHNAKTKPREEQYNSTYRVVSVLTVLHQDWLNGKEYKCKV |
| LALAPG, and | SNKALGAPIEKTISKAKGQPREPQVYTLPPSRDELTKNQVSLSCAVKG |
| M428L and N434S | FYPSDIAVEWESFGTEWSNYKTTPPVLDSDGSFFLVSKLTVSKEEWQQ |
| mutations | GFVFSCSVLHEALHSHYTQKSLSLSPGK (SEQ ID NO: 2472) |
| Clone CH3C.35.23.2 | APELLGGPSVFLFPPKPKDTLMISRTPEVTCVVVDVSHEDPEVKFNWY |
| with M428L and | VDGVEVHNAKTKPREEQYNSTYRVVSVLTVLHQDWLNGKEYKCKV |
| N434S mutations | SNKALPAPIEKTISKAKGQPREPQVYTLPPSRDELTKNQVSLTCLVKGF |
| | YPSDIAVEWESYGTEWANYKTTPPVLDSDGSFFLYSKLTVTKEEWQQ |
| | GFVFSCSVLHEALHSHYTQKSLSLSPGK (SEQ ID NO: 2473) |
| Clone CH3C.35.23.2 | APELLGGPSVFLFPPKPKDTLMISRTPEVTCVVVDVSHEDPEVKFNWY |
| with knob and M428L | VDGVEVHNAKTKPREEQYNSTYRVVSVLTVLHQDWLNGKEYKCKV |
| and N4345 mutations | SNKALPAPIEKTISKAKGQPREPQVYTLPPSRDELTKNQVSLWCLVKG |
| | FYPSDIAVEWESYGTEWANYKTTPPVLDSDGSFFLYSKLTVTKEEWQ |
| | QGFVFSCSVLHEALHSHYTQKSLSLSPGK (SEQ ID NO: 2474) |
| Clone CH3C.35.23.2 | APEAAGGPSVFLFPPKPKDTLMISRTPEVTCVVVDVSHEDPEVKFNWY |
| with knob, LALA, | VDGVEVHNAKTKPREEQYNSTYRVVSVLTVLHQDWLNGKEYKCKV |
| and M428L and | SNKALPAPIEKTISKAKGQPREPQVYTLPPSRDELTKNQVSLWCLVKG |
| N434S mutations | FYPSDIAVEWESYGTEWANYKTTPPVLDSDGSFFLYSKLTVTKEEWQ |
| | QGFVFSCSVLHEALHSHYTQKSLSLSPGK (SEQ ID NO: 2475) |
| Clone CH3C.35.23.2 | APEAAGGPSVFLFPPKPKDTLMISRTPEVTCVVVDVSHEDPEVKFNWY |
| with knob, LALAPG, | VDGVEVHNAKTKPREEQYNSTYRVVSVLTVLHQDWLNGKEYKCKV |
| and M428L and | SNKALGAPIEKTISKAKGQPREPQVYTLPPSRDELTKNQVSLWCLVKG |
| N434S mutations | FYPSDIAVEWESYGTEWANYKTTPPVLDSDGSFFLYSKLTVTKEEWQ |
| | QGFVFSCSVLHEALHSHYTQKSLSLSPGK (SEQ ID NO: 2476) |
| Clone CH3C.35.23.2 | APELLGGPSVFLFPPKPKDTLMISRTPEVTCVVVDVSHEDPEVKFNWY |
| with hole and M428L | VDGVEVHNAKTKPREEQYNSTYRVVSVLTVLHQDWLNGKEYKCKV |
| and N4345 mutations | SNKALPAPIEKTISKAKGQPREPQVYTLPPSRDELTKNQVSLSCAVKGF |
| | YPSDIAVEWESYGTEWANYKTTPPVLDSDGSFFLVSKLTVTKEEWQQ |
| | GFVFSCSVLHEALHSHYTQKSLSLSPGK (SEQ ID NO: 2477) |
| Clone CH3C.35.23.2 | APEAAGGPSVFLFPPKPKDTLMISRTPEVTCVVVDVSHEDPEVKFNWY |
| with hole. LALA, and | VDGVEVHNAKTKPREEQYNSTYRVVSVLTVLHQDWLNGKEYKCKV |
| M428L and N434S | SNKALPAPIEKTISKAKGQPREPQVYTLPPSRDELTKNQVSLSCAVKGF |
| mutations | YPSDIAVEWESYGTEWANYKTTPPVLDSDGSFFLVSKLTVTKEEWQQ |
| | GFVFSCSVLHEALHSHYTQKSLSLSPGK (SEQ ID NO: 2478) |
| Clone CH3C.35.23.2 | APEAAGGPSVFLFPPKPKDTLMISRTPEVTCVVVDVSHEDPEVKFNWY |
| with hole, LALAPG, | VDGVEVHNAKTKPREEQYNSTYRVVSVLTVLHQDWLNGKEYKCKV |
| and M428L and | SNKALGAPIEKTISKAKGQPREPQVYTLPPSRDELTKNQVSLSCAVKG |
| N434S mutations | FYPSDIAVEWESYGTEWANYKTTPPVLDSDGSFFLVSKLTVTKEEWQ |
| | QGFVFSCSVLHEALHSHYTQKSLSLSPGK (SEQ ID NO: 2479) |
| Clone | APELLGGPSVFLFPPKPKDTLMISRTPEVTCVVVDVSHEDPEVKFNWY |
| CH3C.35.23.2.1 with | VDGVEVHNAKTKPREEQYNSTYRVVSVLTVLHQDWLNGKEYKCKV |
| M428L and N4345 | SNKALPAPIEKTISKAKGQPREPQVYTLPPSRDELTKNQVSLTCLVKGF |
| mutations | YPSDIAVEWESYGTEWANYKTTPPVLDSDGSFFLYSKLTVSKSEWQQ |
| | GFVFSCSVLHEALHSHYTQKSLSLSPGK (SEQ ID NO: 2480) |
| Clone | APELLGGPSVFLFPPKPKDTLMISRTPEVTCVVVDVSHEDPEVKFNWY |
| CH3C.35.23.2.1 with | VDGVEVHNAKTKPREEQYNSTYRVVSVLTVLHQDWLNGKEYKCKV |
| knob and M428L and | SNKALPAPIEKTISKAKGQPREPQVYTLPPSRDELTKNQVSLWCLVKG |
| N434S mutations | FYPSDIAVEWESYGTEWANYKTTPPVLDSDGSFFLYSKLTVSKSEWQ |
| | QGFVFSCSVLHEALHSHYTQKSLSLSPGK (SEQ ID NO: 2481) |
| Clone | APEAAGGPSVFLFPPKPKDTLMISRTPEVTCVVVDVSHEDPEVKFNWY |
| CH3C.35.23.2.1 with | VDGVEVHNAKTKPREEQYNSTYRVVSVLTVLHQDWLNGKEYKCKV |
| knob, LALA, and | SNKALPAPIEKTISKAKGQPREPQVYTLPPSRDELTKNQVSLWCLVKG |
| M428L and N434 S | FYPSDIAVEWESYGTEWANYKTTPPVLDSDGSFFLYSKLTVSKSEWQ |
| mutations | QGFVFSCSVLHEALHSHYTQKSLSLSPGK (SEQ ID NO: 2482) |
| Clone | APEAAGGPSVFLFPPKPKDTLMISRTPEVTCVVVDVSHEDPEVKFNWY |
| CH3C.35.23.2.1 with | VDGVEVHNAKTKPREEQYNSTYRVVSVLTVLHQDWLNGKEYKCKV |
| knob, LALAPG, and | SNKALGAPIEKTISKAKGQPREPQVYTLPPSRDELTKNQVSLWCLVKG |
| M428L and N434S | FYPSDIAVEWESYGTEWANYKTTPPVLDSDGSFFLYSKLTVSKSEWQ |
| mutations | QGFVFSCSVLHEALHSHYTQKSLSLSPGK (SEQ ID NO: 2483) |
| Clone | APELLGGPSVFLFPPKPKDTLMISRTPEVTCVVVDVSHEDPEVKFNWY |
| CH3C.35.23.2.1 with | VDGVEVHNAKTKPREEQYNSTYRVVSVLTVLHQDWLNGKEYKCKV |
| hole and M428L and | SNKALPAPIEKTISKAKGQPREPQVYTLPPSRDELTKNQVSLSCAVKGF |
| N434S mutations | YPSDIAVEWESYGTEWANYKTTPPVLDSDGSFFLVSKLTVSKSEWQQ |
| | GFVFSCSVLHEALHSHYTQKSLSLSPGK (SEQ ID NO: 2484) |
| Clone | APEAAGGPSVFLFPPKPKDTLMISRTPEVTCVVVDVSHEDPEVKFNWY |
| CH3C.35.23.2.1 with | VDGVEVHNAKTKPREEQYNSTYRVVSVLTVLHQDWLNGKEYKCKV |
| hole, LALA, and | SNKALPAPIEKTISKAKGQPREPQVYTLPPSRDELTKNQVSLSCAVKGF |
| M428L and N434S | YPSDIAVEWESYGTEWANYKTTPPVLDSDGSFFLVSKLTVSKSEWQQ |
| mutations | GFVFSCSVLHEALHSHYTQKSLSLSPGK (SEQ ID NO: 2485) |
| Clone | APEAAGGPSVFLFPPKPKDTLMISRTPEVTCVVVDVSHEDPEVKFNWY |
| CH3C.35.23.2.1 with | VDGVEVHNAKTKPREEQYNSTYRVVSVLTVLHQDWLNGKEYKCKV |
| hole, LALAPG, and | SNKALGAPIEKTISKAKGQPREPQVYTLPPSRDELTKNQVSLSCAVKG |
| M428L and N434S | FYPSDIAVEWESYGTEWANYKTTPPVLDSDGSFFLVSKLTVSKSEWQ |
| mutations | QGFVFSCSVLHEALHSHYTQKSLSLSPGK (SEQ ID NO: 2486) |
| Clone CH3C.35.23.3 | APELLGGPSVFLFPPKPKDTLMISRTPEVTCVVVDVSHEDPEVKFNWY |
| with M428L and | VDGVEVHNAKTKPREEQYNSTYRVVSVLTVLHQDWLNGKEYKCKV |
| N434S mutations | SNKALPAPIEKTISKAKGQPREPQVYTLPPSRDELTKNQVSLTCLVKGF |
| | YPSDIAVEWESYGTEWVNYKTTPPVLDSDGSFFLYSKLTVTKEEWQQ |
| | GFVFSCSVLHEALHSHYTQKSLSLSPGK (SEQ ID NO: 2487) |
| Clone CH3C.35.23.3 | APELLGGPSVFLFPPKPKDTLMISRTPEVTCVVVDVSHEDPEVKFNWY |
| with knob and M428L | VDGVEVHNAKTKPREEQYNSTYRVVSVLTVLHQDWLNGKEYKCKV |
| and N4345 mutations | SNKALPAPIEKTISKAKGQPREPQVYTLPPSRDELTKNQVSLWCLVKG |
| | FYPSDIAVEWESYGTEWVNYKTTPPVLDSDGSFFLYSKLTVTKEEWQ |
| | QGFVFSCSVLHEALHSHYTQKSLSLSPGK (SEQ ID NO: 2488) |
| Clone CH3C.35.23.3 | APEAAGGPSVFLFPPKPKDTLMISRTPEVTCVVVDVSHEDPEVKFNWY |
| with knob, LALA, | VDGVEVHNAKTKPREEQYNSTYRVVSVLTVLHQDWLNGKEYKCKV |
| and M428L and | SNKALPAPIEKTISKAKGQPREPQVYTLPPSRDELTKNQVSLWCLVKG |
| N434S mutations | FYPSDIAVEWESYGTEWVNYKTTPPVLDSDGSFFLYSKLTVTKEEWQ |
| | QGFVFSCSVLHEALHSHYTQKSLSLSPGK (SEQ ID NO: 2489) |
| Clone CH3C.35.23.3 | APEAAGGPSVFLFPPKPKDTLMISRTPEVTCVVVDVSHEDPEVKFNWY |
| with knob, LALAPG, | VDGVEVHNAKTKPREEQYNSTYRVVSVLTVLHQDWLNGKEYKCKV |
| and M428L and | SNKALGAPIEKTISKAKGQPREPQVYTLPPSRDELTKNQVSLWCLVKG |
| N434S mutations | FYPSDIAVEWESYGTEWVNYKTTPPVLDSDGSFFLYSKLTVTKEEWQ |
| | QGFVFSCSVLHEALHSHYTQKSLSLSPGK (SEQ ID NO: 2490) |
| Clone CH3C.35.23.3 | APELLGGPSVFLFPPKPKDTLMISRTPEVTCVVVDVSHEDPEVKFNWY |
| with hole and M428L | VDGVEVHNAKTKPREEQYNSTYRVVSVLTVLHQDWLNGKEYKCKV |
| and N4345 mutations | SNKALPAPIEKTISKAKGQPREPQVYTLPPSRDELTKNQVSLSCAVKGF |
| | YPSDIAVEWESYGTEWVNYKTTPPVLDSDGSFFLVSKLTVTKEEWQQ |
| | GFVFSCSVLHEALHSHYTQKSLSLSPGK (SEQ ID NO: 2491) |
| Clone CH3C.35.23.3 | APEAAGGPSVFLFPPKPKDTLMISRTPEVTCVVVDVSHEDPEVKFNWY |
| with hole. LALA, and | VDGVEVHNAKTKPREEQYNSTYRVVSVLTVLHQDWLNGKEYKCKV |
| M428L and N4345 | SNKALPAPIEKTISKAKGQPREPQVYTLPPSRDELTKNQVSLSCAVKGF |
| mutations | YPSDIAVEWESYGTEWVNYKTTPPVLDSDGSFFLVSKLTVTKEEWQQ |
| | GFVFSCSVLHEALHSHYTQKSLSLSPGK (SEQ ID NO: 2492) |
| Clone CH3C.35.23.3 | APEAAGGPSVFLFPPKPKDTLMISRTPEVTCVVVDVSHEDPEVKFNWY |
| with hole, LALAPG, | VDGVEVHNAKTKPREEQYNSTYRVVSVLTVLHQDWLNGKEYKCKV |
| and M428L and | SNKALGAPIEKTISKAKGQPREPQVYTLPPSRDELTKNQVSLSCAVKG |
| N434S mutations | FYPSDIAVEWESYGTEWVNYKTTPPVLDSDGSFFLVSKLTVTKEEWQ |
| | QGFVFSCSVLHEALHSHYTQKSLSLSPGK (SEQ ID NO: 2493) |
| Clone CH3C.35.23.4 | APELLGGPSVFLFPPKPKDTLMISRTPEVTCVVVDVSHEDPEVKFNWY |
| with M428L and | VDGVEVHNAKTKPREEQYNSTYRVVSVLTVLHQDWLNGKEYKCKV |
| N434S mutations | SNKALPAPIEKTISKAKGQPREPQVYTLPPSRDELTKNQVSLTCLVKGF |
| | YPSDIAVEWESYGTEWSNYKTTPPVLDSDGSFFLYSKLTVSKEEWQQ |
| | GFVFSCSVLHEALHSHYTQKSLSLSPGK (SEQ ID NO: 2494) |
| Clone CH3C.35.23.4 | APELLGGPSVFLFPPKPKDTLMISRTPEVTCVVVDVSHEDPEVKFNWY |
| with knob and M428L | VDGVEVHNAKTKPREEQYNSTYRVVSVLTVLHQDWLNGKEYKCKV |
| and N4345 mutations | SNKALPAPIEKTISKAKGQPREPQVYTLPPSRDELTKNQVSLWCLVKG |
| | FYPSDIAVEWESYGTEWSNYKTTPPVLDSDGSFFLYSKLTVSKEEWQQ |
| | GFVFSCSVLHEALHSHYTQKSLSLSPGK (SEQ ID NO: 2495) |
| Clone CH3C.35.23.4 | APEAAGGPSVFLFPPKPKDTLMISRTPEVTCVVVDVSHEDPEVKFNWY |
| with knob, LALA, | VDGVEVHNAKTKPREEQYNSTYRVVSVLTVLHQDWLNGKEYKCKV |
| and M428L and | SNKALPAPIEKTISKAKGQPREPQVYTLPPSRDELTKNQVSLWCLVKG |
| N434S mutations | FYPSDIAVEWESYGTEWSNYKTTPPVLDSDGSFFLYSKLTVSKEEWQQ |
| | GFVFSCSVLHEALHSHYTQKSLSLSPGK (SEQ ID NO: 2496) |
| Clone CH3C.35.23.4 | APEAAGGPSVFLFPPKPKDTLMISRTPEVTCVVVDVSHEDPEVKFNWY |
| with knob, LALAPG, | VDGVEVHNAKTKPREEQYNSTYRVVSVLTVLHQDWLNGKEYKCKV |
| and M428L and | SNKALGAPIEKTISKAKGQPREPQVYTLPPSRDELTKNQVSLWCLVKG |
| N434S mutations | FYPSDIAVEWESYGTEWSNYKTTPPVLDSDGSFFLYSKLTVSKEEWQQ |
| | GFVFSCSVLHEALHSHYTQKSLSLSPGK (SEQ ID NO: 2497) |
| Clone CH3C.35.23.4 | APELLGGPSVFLFPPKPKDTLMISRTPEVTCVVVDVSHEDPEVKFNWY |
| with hole and M428L | VDGVEVHNAKTKPREEQYNSTYRVVSVLTVLHQDWLNGKEYKCKV |
| and N4345 mutations | SNKALPAPIEKTISKAKGQPREPQVYTLPPSRDELTKNQVSLSCAVKGF |
| | YPSDIAVEWESYGTEWSNYKTTPPVLDSDGSFFLVSKLTVSKEEWQQ |
| | GFVFSCSVLHEALHSHYTQKSLSLSPGK (SEQ ID NO: 2498) |
| Clone CH3C.35.23.4 | APEAAGGPSVFLFPPKPKDTLMISRTPEVTCVVVDVSHEDPEVKFNWY |
| with hole, LALA, and | VDGVEVHNAKTKPREEQYNSTYRVVSVLTVLHQDWLNGKEYKCKV |
| M428L and N4345 | SNKALPAPIEKTISKAKGQPREPQVYTLPPSRDELTKNQVSLSCAVKGF |
| mutations | YPSDIAVEWESYGTEWSNYKTTPPVLDSDGSFFLVSKLTVSKEEWQQ |
| | GFVFSCSVLHEALHSHYTQKSLSLSPGK (SEQ ID NO: 2499) |
| Clone CH3C.35.23.4 | APEAAGGPSVFLFPPKPKDTLMISRTPEVTCVVVDVSHEDPEVKFNWY |
| with hole, LALAPG, | VDGVEVHNAKTKPREEQYNSTYRVVSVLTVLHQDWLNGKEYKCKV |
| and M428L and | SNKALGAPIEKTISKAKGQPREPQVYTLPPSRDELTKNQVSLSCAVKG |
| N434S mutations | FYPSDIAVEWESYGTEWSNYKTTPPVLDSDGSFFLVSKLTVSKEEWQQ |
| | GFVFSCSVLHEALHSHYTQKSLSLSPGK (SEQ ID NO: 2500) |
| Fc sequence with hole | APELLGGPSVFLFPPKPKDTLMISRTPEVTCVVVDVSHEDPEVKFNWY |
| and M428L and | VDGVEVHNAKTKPREEQYNSTYRVVSVLTVLHQDWLNGKEYKCKV |
| N434S mutations | SNKALPAPIEKTISKAKGQPREPQVYTLPPSRDELTKNQVSLSCAVKGF |
| | YPSDIAVEWESNGQPENNYKTTPPVLDSDGSFFLVSKLTVDKSRWQQ |
| | GNVFSCSVLHEALHSHYTQKSLSLSPGK (SEQ ID NO: 2501) |
| Fc sequence with | APEAAGGPSVFLFPPKPKDTLMISRTPEVTCVVVDVSHEDPEVKFNWY |
| hole, LALA, and | VDGVEVHNAKTKPREEQYNSTYRVVSVLTVLHQDWLNGKEYKCKV |
| M428L and N4345 | SNKALPAPIEKTISKAKGQPREPQVYTLPPSRDELTKNQVSLSCAVKGF |
| mutations | YPSDIAVEWESNGQPENNYKTTPPVLDSDGSFFLVSKLTVDKSRWQQ |
| | GNVFSCSVLHEALHSHYTQKSLSLSPGK (SEQ ID NO: 2502) |
| Fc sequence with | APELLGGPSVFLFPPKPKDTLMISRTPEVTCVVVDVSHEDPEVKFNWY |
| knob and M428L and | VDGVEVHNAKTKPREEQYNSTYRVVSVLTVLHQDWLNGKEYKCKV |
| N434S mutations | SNKALPAPIEKTISKAKGQPREPQVYTLPPSRDELTKNQVSLWCLVKG |
| | FYPSDIAVEWESNGQPENNYKTTPPVLDSDGSFFLYSKLTVDKSRWQ |
| | QGNVFSCSVLHEALHSHYTQKSLSLSPGK (SEQ ID NO: 2503) |
| Fc sequence with | APEAAGGPSVFLFPPKPKDTLMISRTPEVTCVVVDVSHEDPEVKFNWY |
| knob, LALA, and | VDGVEVHNAKTKPREEQYNSTYRVVSVLTVLHQDWLNGKEYKCKV |
| M428L and N434S | SNKALPAPIEKTISKAKGQPREPQVYTLPPSRDELTKNQVSLWCLVKG |
| mutations | FYPSDIAVEWESNGQPENNYKTTPPVLDSDGSFFLYSKLTVDKSRWQ |
| | QGNVFSCSVLHEALHSHYTQKSLSLSPGK (SEQ ID NO: 2504) |
| SS2_NHis_TREM2 | MPALLSLVSLLSVLLMGCVAETGGSGHHHHHHSGTHNTTVFQGVAG |
| | QSLQVSCPYDSMIKHWGRRKAWCRQLGEKGPCQRVVSTHNLWLLSFL |
| | RRWNGSTAITDDTLGGTLTITLRNLQPHDAGLYQCQSLHGSEADTLR |
| | KVLVEVLADPLDHRDAGDLWFPGESESFEDAHVEHSISRSLLEGEIPFP |
| | PTSAS (SEQ ID NO: 2505) |

In some embodiments, each of the light chain variable regions and each of the heavy chain variable regions disclosed in in the above tables as well as specific combinations thereof and other embodiments of the anti-TREM2 antibody described in the '841 application and herein may be attached to the light chain constant regions (TABLE EN1) and heavy chain constant regions (TABLE EN2) to form complete antibody light and heavy chains, respectively, as further discussed below. Further, each of the generated heavy and light chain sequences may be combined to form a complete antibody structure. It should be understood that the heavy chain and light chain variable regions provided herein can also be attached to other constant domains having different sequences than the exemplary sequences listed herein.

H. PCT Patent Application Publication No. WO2019/118513A1

In some embodiments, the TREM2 agonist is an antibody or an antigen-binding fragment thereof, as described in PCT Patent Application Publication No. WO2019/118513A1 (“the '513 application”), which is incorporated by reference herein, in its entirety.

In some embodiments, the TREM2 binding agent comprises an antibody that comprises a light chain variable domain comprising a CDRL1, CDRL2, and CDRL3, and a heavy chain variable domain comprising a CDRH1, CDRH2, and CDRH3 disclosed in the '513 application specification. In some embodiments, the TREM2 binding agent comprises an antibody that comprises a light chain variable domain and a heavy chain variable domain disclosed in the '513 application specification.

In some embodiments, the antibody comprises a CDR-H1 comprising the sequence set forth in SEQ ID NO:2514, a CDR-H2 comprising the sequence set forth in SEQ ID NO:2515, a CDR-H3 comprising the sequence set forth in SEQ ID NO: 11, a CDR-L1 comprising the sequence set forth in SEQ ID NO:2517, a CDR-L2 comprising the sequence set forth in SEQ ID NO:2518, and a CDR-L3 comprising the sequence set forth in SEQ ID NO:2519.

In some embodiments, the antibody is afucosylated and comprises the VH sequence shown in SEQ ID NO:2506; the VL sequence shown in SEQ ID NO:2507; and an active human IgG1 Fc region.

In some embodiments, the antibody comprises all 3 heavy chain CDRs of the sequence shown in SEQ ID NO:2512 and all 3 light chain CDRs of the sequence shown in SEQ ID NO:2513.

In some embodiments, the antibody comprises an A to T substitution at position 97 of the sequence shown in SEQ ID NO:2512; and a K to R substitution at position 98 of the sequence shown in SEQ ID NO:2512.

In some embodiments, the antibody comprises the VH sequence shown in SEQ ID NO:2506, 2508, or 2510.

In some embodiments, the antibody comprises the VH sequence shown in SEQ ID NO:2506, 2508, or 2510 and the VL sequence shown in SEQ ID NO:2507, 2509, or 2511. In some embodiments, the antibody comprises the VH sequence shown in SEQ ID NO:2506.

In some embodiments, the antibody comprises the VH sequence shown in SEQ ID NO:2506 and the VL sequence shown in SEQ ID NO:2507.

In some embodiments, the antibody is the 37012 antibody (see TABLE H1)

In some embodiments, the antibody is an antibody having a VL, VH, full heavy chain sequence or full light chain sequence disclosed in Table 1A or a CDR sequence as disclosed in Table 1B of PCT Patent Application Publication No. WO2019/118513A1, which are reproduced below as TABLES H1 and H2 respectively.

TABLE H1

| Name | Sequence |
|---|---|
| 37012 VH | EVQLLESGGGLVQPGGSLRLSCAASGFTFSNYYMAWVRQAPGKGLEW |
| | VSSLTNSGGSTYYADSVKGRFTISRDNSKNTLYLQMNSLRAEDTAVYY |
| | CTREWAGSGYFDYWGQGTLVTVSS (SEQ ID NO: 2506) |
| 37012 VL | DIQMTQSPSSLSASVGDRVTITCKASQNVGNNLAWYQQKPGKAPKLLIY |
| | YTSNRFTGVPSRFSGSGSGTDFTLTISSLQPEDFATYYCQRIYNSPWTFG |
| | QGTKLEIK (SEQ ID NO: 2507) |
| 37013VH | EVQLLESGGGLVQPGGSLRLSCAASGFTFSNYYMAWVRQAPGKGLEW |
| | VSSLTNSGGSTYYADSVKGRFTISRDNSKNTLYLQMNSLRAEDTAVYY |
| | CTREWAGSGYFDYWGQGTLVTVSS (SEQ ID NO: 2508) |
| 37013VL | DIQMTQSPSSLSASVGDRVTMTCKASQNVGNNLAWYQQKPGKAPKLL |
| | LYYTSNRFTGVPSRFSGSGSGTDFTLTISSVQPEDFATYYCQRIYNSPWT |
| | FGQGTKLELK (SEQ ID NO: 2509) |
| 37014VH | EVQLLESGGGLVQPGGSLRLSCAASGFTFSNYYMAWVRQAPGKGLEW |
| | VASLTNSGGSTYYADSVKGRFTLSRDNSKNTLYLQMNSLRAEDTAVYY |
| | CTREWAGSGYFDYWGQGTLVTVSS (SEQ ID NO: 2510) |
| 37014VL | DIQMTQSPSSLSASVGDRVTITCKASQNVGNNLAWYQQKPGKAPKLLIY |
| | YTSNRFTGVPSRFSGSGSGTDFTLTISSLQPEDFATYYCQRIYNSPWTFG |
| | QGTKLEIK (SEQ ID NO: 2511) |
| 37017VH | EVQLLESGGGLVQPGGSLRLSCAASGFTFSNYYMAWVRQAPGKGLEW |
| | VSSLTNSGGSTYYADSVKGRFTISRDNSKNTLYLQMNSLRAEDTAVYY |
| | CAKEWAGSGYFDYWGQGTLVTVSS (SEQ ID NO: 2512) |
| 37017VL | DIQMTQSPSSLSASVGDRVTITCKASQNVGNNLAWYQQKPGKAPKLLIY |
| | YTSNRFTGVPSRFSGSGSGTDFTLTISSLQPEDFATYYCQRIYNSPWTFG |
| | QGTKLEIK (SEQ ID NO: 2513) |
| Full 37012_H | EVQLLESGGGLVQPGGSLRLSCAASGFTFSNYYMAWVRQAPGKGLEW |
| | VSSLTNSGGSTYYADSVKGRFTISRDNSKNTLYLQMNSLRAEDTAVYY |
| | CTREWAGSGYFDYWGQGTLVTVSSASTKGPSVFPLAPSSKSTSGGTAA |
| | LGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPELLGGPS |
| | VFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESN |
| | GQPENNYKTTPPVLDSDGSFFLYSKLTVDKSRWQQGNVFSCSVMHEAL |
| | HNHYTQKSLSLSPGK (SEQ ID NO: 2529) |
| Full 37012 L | DIQMTQSPSSLSASVGDRVTITCKASQNVGNNLAWYQQKPGKAPKLLIY |
| | YTSNRFTGVPSRFSGSGSGTDFTLTISSLQPEDFATYYCQRIYNSPWTFG |
| | QGTKLEIKRTVAAPSVFIFPPSDEQLKSGTASVVCLLNNFYPREAKVQW |
| | KVDNALQSGNSQESVTEQDSKDSTYSLSSTLTLSKADYEKHKVYACEV |
| | THQGLSSPVTKSFNRGEC (SEQ ID NO: 2530) |
| Full 37013_H | EVQLLESGGGLVQPGGSLRLSCAASGFTFSNYYMAWVRQAPGKGLEW |
| | VSSLTNSGGSTYYADSVKGRFTISRDNSKNTLYLQMNSLRAEDTAVYY |
| | CTREWAGSGYFDYWGQGTLVTVSSASTKGPSVFPLAPSSKSTSGGTAA |
| | LGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPELLGGPS |
| | VFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESN |
| | GQPENNYKTTPPVLDSDGSFFLYSKLTVDKSRWQQGNVFSCSVMHEAL |
| | HNHYTQKSLSLSPGK (SEQ ID NO: 2531) |
| Full 37013 L | DIQMTQSPSSLSASVGDRVTMTCKASQNVGNNLAWYQQKPGKAPKLL |
| | LYYTSNRFTGVPSRFSGSGSGTDFTLTISSVQPEDFATYYCQRIYNSPWT |
| | FGQGTKLELKRTVAAPSVFIFPPSDEQLKSGTASVVCLLNNFYPREAKV |
| | QWKVDNALQSGNSQESVTEQDSKDSTYSLSSTLTLSKADYEKHKVYAC |
| | EVTHQGLSSPVTKSFNRGEC (SEQ ID NO: 2532) |
| Full 37017_H | EVQLLESGGGLVQPGGSLRLSCAASGFTFSNYYMAWVRQAPGKGLEW |
| | VASLTNSGGSTYYADSVKGRFTLSRDNSKNTLYLQMNSLRAEDTAVYY |
| | CTREWAGSGYFDYWGQGTLVTVSSASTKGPSVFPLAPSSKSTSGGTAA |
| | LGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPELLGGPS |
| | VFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESN |
| | GQPENNYKTTPPVLDSDGSFFLYSKLTVDKSRWQQGNVFSCSVMHEAL |
| | HNHYTQKSLSLSPGK (SEQ ID NO: 2533) |
| Full 37017_L | DIQMTQSPSSLSASVGDRVTITCKASQNVGNNLAWYQQKPGKAPKLLIY |
| | YTSNRFTGVPSRFSGSGSGTDFTLTISSLQPEDFATYYCQRIYNSPWTFG |
| | QGTKLEIKRTVAAPSVFIFPPSDEQLKSGTASVVCLLNNFYPREAKVQW |
| | KVDNALQSGNSQESVTEQDSKDSTYSLSSTLTLSKADYEKHKVYACEV |
| | THQGLSSPVTKSFNRGEC (SEQ ID NO: 2534) |
| Full 37017_H | EVQLLESGGGLVQPGGSLRLSCAASGFTFSNYYMAWVRQAPGKGLEW |
| | VSSLTNSGGSTYYADSVKGRFTISRDNSKNTLYLQMNSLRAEDTAVYY |
| | CAKEWAGSGYFDYWGQGTLVTVSSASTKGPSVFPLAPSSKSTSGGTAA |
| | LGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPELLGGPS |
| | VFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESN |
| | GQPENNYKTTPPVLDSDGSFFLYSKLTVDKSRWQQGNVFSCSVMHEAL |
| | HNHYTQKSLSLSPGK (SEQ ID NO: 2535) |
| Full 37017_L | DIQMTQSPSSLSASVGDRVTITCKASQNVGNNLAWYQQKPGKAPKLLIY |
| | YTSNRFTGVPSRFSGSGSGTDFTLTISSLQPEDFATYYCQRIYNSPWTFG |
| | QGTKLEIKRTVAAPSVFIFPPSDEQLKSGTASVVCLLNNFYPREAKVQW |
| | KVDNALQSGNSQESVTEQDSKDSTYSLSSTLTLSKADYEKHKVYACEV |
| | THQGLSSPVTKSFNRGEC (SEQ ID NO: 2536) |

TABLE H2
CDR's of humanized antibodies

| CDR | Sequence |
|---|---|
| CDR-H1 | FSNYYMA (SEQ ID NO: 2514) |
| CDR-H2 | SLTNSGGSTY (SEQ ID NO: 2515) |
| CDR-H3 | EWAGSGY (SEQ ID NO: 2516) |
| CDR-L1 | NVGNNLA (SEQ ID NO: 2517) |
| CDR-L2 | YTSNRFT (SEQ ID NO: 2518) |
| CDR-L3 | RIYNSPW (SEQ ID NO: 2519) |

In some embodiments, each of the light chain variable regions and each of the heavy chain variable regions disclosed in in the above tables as well as specific combinations thereof and other embodiments of the anti-TREM2 antibody described in the '513 application and herein may be attached to the light chain constant regions (TABLE EN1) and heavy chain constant regions (TABLE EN2) to form complete antibody light and heavy chains, respectively, as further discussed below. Further, each of the generated heavy and light chain sequences may be combined to form a complete antibody structure. It should be understood that the heavy chain and light chain variable regions provided herein can also be attached to other constant domains having different sequences than the exemplary sequences listed herein.

I. PCT Patent Application Publication No. WO2020/055975A1

In some embodiments, the TREM2 agonist is an antibody or an antigen-binding fragment thereof, as described in PCT Patent Application Publication No. WO2020/055975A1 (“the '975 application”), which is incorporated by reference herein, in its entirety.

In some embodiments, the TREM2 binding agent comprises an antibody that comprises a light chain variable domain comprising a CDRL1, CDRL2, and CDRL3, and a heavy chain variable domain comprising a CDRH1, CDRH2, and CDRH3 disclosed in the '975 application specification. In some embodiments, the TREM2 binding agent comprises an antibody that comprises a light chain variable domain and a heavy chain variable domain disclosed in the '975 application specification.

In some embodiments, the antibody comprises (a) a light chain variable region comprising an L1 derived from SEQ ID NO:2539, an L2 derived from SEQ ID NO:2539, an L3 derived from of SEQ ID NO:2539, or any combination thereof; and/or (b) a heavy chain variable region comprising an H1 derived from SEQ ID NO:2540, an H2 derived from SEQ ID NO:2540, an H3 derived from SEQ ID NO:2540, or any combination thereof.

In some embodiments, the antibody comprises (a) a light chain variable region comprising an L1 of SEQ ID NO:2541, an L2 comprising the amino acid sequence IVS, an L3 of SEQ ID NO:2542, or any combination thereof; and/or (b) a heavy chain variable region comprising an H1 comprising SEQ ID NO:2543, an H2 comprising SEQ ID NO:2544, an H3 comprising SEQ ID NO:2545, or any combination thereof.

In some embodiments, the antibody comprises (a) a light chain variable region comprising an L1 derived from SEQ ID NO:2546, an L2 derived from SEQ ID NO:2546, an L3 derived from of SEQ ID NO:2546, or any combination thereof; and/or (b) a heavy chain variable region comprising an H1 derived from SEQ ID NO:2547, an H2 derived from SEQ ID NO:2547, an H3 derived from of SEQ ID NO:2547, or any combination thereof.

In some embodiments, the antibody comprises (a) a light chain variable region comprising an L1 of SEQ ID NO:2548, an L2 comprising the amino acid sequence KVS, an L3 of SEQ ID NO:2549, or any combination thereof; and/or (b) a heavy chain variable region comprising an H1 comprising SEQ ID NO:2550, an H2 comprising SEQ ID NO:2551, an H3 comprising SEQ ID NO:2552, or any combination thereof.

In some embodiments, the antibody comprises (a) a light chain variable region comprising an L1 derived from SEQ ID NO:2553, an L2 derived from SEQ ID NO:2553, an L3 derived from of SEQ ID NO:2553, or any combination thereof; and/or (b) a heavy chain variable region comprising an H1 derived from SEQ ID NO:2554, an H2 derived from SEQ ID NO:2554, an H3 derived from of SEQ ID NO:2554, or any combination thereof.

In some embodiments, the antibody comprises (a) a light chain variable region comprising an L1 of SEQ ID NO:2555, an L2 comprising the amino acid sequence KVS, an L3 of SEQ ID NO:2556, or any combination thereof; and/or (b) a heavy chain variable region comprising an H1 comprising SEQ ID NO:2557, an H2 comprising SEQ ID NO:2558, an H3 comprising SEQ ID NO:2559, or any combination thereof.

In some embodiments, the antibody comprises (a) a light chain variable region comprising an L1 derived from SEQ ID NO:2560, an L2 derived from SEQ ID NO:2560, an L3 derived from of SEQ ID NO:2560, or any combination thereof; and/or (b) a heavy chain variable region comprising an H1 derived from SEQ ID NO:2561, an H2 derived from SEQ ID NO:2561, an H3 derived from of SEQ ID NO:2561, or any combination thereof.

In some embodiments, the antibody comprises (a) a light chain variable region comprising an L1 of SEQ ID NO:2562, an L2 comprising the amino acid sequence KVS, an L3 of SEQ ID NO:2563, or any combination thereof; and/or (b) a heavy chain variable region comprising an H1 comprising SEQ ID NO:2564, an H2 comprising SEQ ID NO:2565, an H3 comprising SEQ ID NO:2566, or any combination thereof.

In some embodiments, the antibody comprises (a) a light chain variable region comprising an L1 derived from SEQ ID NO:2567, an L2 derived from SEQ ID NO:2567, an L3 derived from of SEQ ID NO:2567, or any combination thereof; and/or (b) a heavy chain variable region comprising an H1 derived from SEQ ID NO:2568, an H2 derived from SEQ ID NO:2568, an H3 derived from of SEQ ID NO:2568, or any combination thereof. Compositions comprising the antibody, including but not limited to pharmaceutical compositions, are contemplated herein. In certain embodiments the antibody is a humanized antibody.

In some embodiments, the antibody comprises (a) a light chain variable region comprising an L1 of SEQ ID NO:2569, an L2 comprising the amino acid sequence KVS, an L3 of SEQ ID NO:2570, or any combination thereof; and/or (b) a heavy chain variable region comprising an H1 comprising SEQ ID NO:2571, an H2 comprising SEQ ID NO:2572, an H3 comprising SEQ ID NO:2573, or any combination thereof.

In some embodiments, the antibody comprises (a) a light chain variable region comprising an L1 derived from SEQ ID NO:2574, an L2 derived from SEQ ID NO:2574, an L3 derived from of SEQ ID NO:2574, or any combination thereof; and/or (b) a heavy chain variable region comprising an H1 derived from SEQ ID NO:2575, an H2 derived from SEQ ID NO:2575, an H3 derived from of SEQ ID NO:2575, or any combination thereof.

In some embodiments, the antibody comprises (a) a light chain variable region comprising an L1 of SEQ ID NO:2576, an L2 comprising the amino acid sequence WAS, an L3 of SEQ ID NO:2577, or any combination thereof; and/or (b) a heavy chain variable region comprising an H1 comprising SEQ ID NO:2578, an H2 comprising SEQ ID NO:2579, an H3 comprising SEQ ID NO:2580, or any combination thereof.

In some embodiments, the antibody is HJ23.4, HJ23.7, HJ23.8, HJ23.9, HJ23.10, or HJ23.13. In some embodiments, the antibody is a humanized antibody derived from HJ23.4, HJ23.7, HJ23.8, HJ23.9, HJ23.10, or HJ23.13. The accession number for the hybridoma that produced antibodies HJ23.4, HJ23.7, HJ23.8, HJ23.9, HJ23.10, and HJ23.13, and their respective light chain variable and heavy chain variable regions are noted in TABLE I1:

TABLE I1

| Antibody (ATCC # | Light chain | Heavy chain |
|---|---|---|
| of the hybridoma) | variable region | variable region |
| HJ23.4 (PTA-125168) | SEQ ID NO: 2539 | SEQ ID NO: 2540 |
| HJ23.7 (PTA-125169) | SEQ ID NO: 2546 | SEQ ID NO: 2547 |
| HJ23.8 (PTA-125170) | SEQ ID NO: 2552 | SEQ ID NO: 2553 |
| HJ23.9 (PTA-125171) | SEQ ID NO: 2561 | SEQ ID NO: 2562 |
| HJ23.10 (PTA-125172) | SEQ ID NO: 2567 | SEQ ID NO: 2568 |
| HJ23.13 (PTA-125173) | SEQ ID NO: 2574 | SEQ ID NO: 2575 |

In some embodiments, the antibody is an antibody disclosed in Tables A and B or the summary table appended to Example 2 of PCT Patent Application Publication No. WO2020/055975A1, reproduced below as TABLES I2-4.

TABLE I2

| | Light Chain HVR | | | Heavy Chain HVR | | |
|---|---|---|---|---|---|---|
| Antibody | L1 | L2 | L3 | H1 | H2 | H3 |
| 1 | SEQ ID NO: 2541 | | | | | |
| 2 | SEQ ID NO: 2541 | IVS | | | | |
| 3 | SEQ ID NO: 2541 | IVS | SEQ ID NO: 2542 | | | |
| 4 | | IVS | | | | |
| 5 | | IVS | SEQ ID NO: 2542 | | | |
| 6 | | | SEQ ID NO: 2542 | | | |
| 7 | SEQ ID NO: 2541 | | SEQ ID NO: 2542 | | | |
| 8 | | | | SEQ ID NO: 2543 | | |
| 9 | | | | SEQ ID NO: 2543 | SEQ ID NO: 2544 | |
| 10 | | | | SEQ ID NO: 2543 | SEQ ID NO: 2544 | SEQ ID NO: 2545 |
| 11 | | | | | SEQ ID NO: 2544 | |
| 12 | | | | | SEQ ID NO: 2544 | SEQ ID NO: 2545 |
| 13 | | | | | | SEQ ID NO: 2545 |
| 14 | | | | SEQ ID NO: 2543 | | SEQ ID NO: 2545 |
| 15 | SEQ ID NO: 2541 | | | SEQ ID NO: 2543 | | |
| 16 | SEQ ID NO: 2541 | | | SEQ ID NO: 2543 | SEQ ID NO: 2544 | |
| 17 | SEQ ID NO: 2541 | | | SEQ ID NO: 2543 | SEQ ID NO: 2544 | SEQ ID NO: 2545 |
| 18 | SEQ ID NO: 2541 | | | | SEQ ID NO: 2544 | |
| 19 | SEQ ID NO: 2541 | | | | SEQ ID NO: 2544 | SEQ ID NO: 2545 |
| 20 | SEQ ID NO: 2541 | | | | | SEQ ID NO: 2545 |
| 21 | SEQ ID NO: 2541 | | | SEQ ID NO: 2543 | | SEQ ID NO: 2545 |
| 22 | SEQ ID NO: 2541 | IVS | | SEQ ID NO: 2543 | | |
| 23 | SEQ ID NO: 2541 | IVS | | SEQ ID NO: 2543 | SEQ ID NO: 2544 | |
| 24 | SEQ ID NO: 2541 | IVS | | SEQ ID NO: 2543 | SEQ ID NO: 2544 | SEQ ID NO: 2545 |
| 25 | SEQ ID NO: 2541 | IVS | | | SEQ ID NO: 2544 | |
| 26 | SEQ ID NO: 2541 | IVS | | | SEQ ID NO: 2544 | SEQ ID NO: 2545 |
| 27 | SEQ ID NO: 2541 | IVS | | | | SEQ ID NO: 2545 |
| 28 | SEQ ID NO: 2541 | IVS | | SEQ ID NO: 2543 | | SEQ ID NO: 2545 |
| 29 | SEQ ID NO: 2541 | IVS | SEQ ID NO: 2542 | SEQ ID NO: 2543 | | |
| 30 | SEQ ID NO: 2541 | IVS | SEQ ID NO: 2542 | SEQ ID NO: 2543 | SEQ ID NO: 2544 | |
| 31 | SEQ ID NO: 2541 | IVS | SEQ ID NO: 2542 | SEQ ID NO: 2543 | SEQ ID NO: 2544 | SEQ ID NO: 2545 |
| 32 | SEQ ID NO: 2541 | IVS | SEQ ID NO: 2542 | | SEQ ID NO: 2544 | |
| 33 | SEQ ID NO: 2541 | IVS | SEQ ID NO: 2542 | | SEQ ID NO: 2544 | SEQ ID NO: 2545 |
| 34 | SEQ ID NO: 2541 | IVS | SEQ ID NO: 2542 | SEQ ID NO: 2543 | | SEQ ID NO: 2545 |
| 35 | SEQ ID NO: 2541 | IVS | SEQ ID NO: 2542 | | | SEQ ID NO: 2545 |
| 36 | | IVS | | SEQ ID NO: 2543 | | |
| 37 | | IVS | | SEQ ID NO: 2543 | SEQ ID NO: 2544 | |
| 38 | | IVS | | SEQ ID NO: 2543 | SEQ ID NO: 2544 | SEQ ID NO: 2545 |
| 39 | | IVS | | | SEQ ID NO: 2544 | |
| 40 | | IVS | | | SEQ ID NO: 2544 | SEQ ID NO: 2545 |
| 41 | | IVS | | | | SEQ ID NO: 2545 |
| 42 | | IVS | | SEQ ID NO: 2543 | | SEQ ID NO: 2545 |
| 43 | | IVS | SEQ ID NO: 2542 | SEQ ID NO: 2543 | | |
| 44 | | IVS | SEQ ID NO: 2542 | SEQ ID NO: 2543 | SEQ ID NO: 2544 | |
| 45 | | IVS | SEQ ID NO: 2542 | SEQ ID NO: 2543 | SEQ ID NO: 2544 | SEQ ID NO: 2545 |
| 46 | | IVS | SEQ ID NO: 2542 | | SEQ ID NO: 2544 | |
| 47 | | IVS | SEQ ID NO: 2542 | | SEQ ID NO: 2544 | SEQ ID NO: 2545 |
| 48 | | IVS | SEQ ID NO: 2542 | | | SEQ ID NO: 2545 |
| 49 | | IVS | SEQ ID NO: 2542 | SEQ ID NO: 2543 | | SEQ ID NO: 2545 |
| 50 | | | SEQ ID NO: 2542 | SEQ ID NO: 2543 | | |
| 51 | | | SEQ ID NO: 2542 | SEQ ID NO: 2543 | SEQ ID NO: 2544 | |
| 52 | | | SEQ ID NO: 2542 | SEQ ID NO: 2543 | SEQ ID NO: 2544 | SEQ ID NO: 2545 |
| 53 | | | SEQ ID NO: 2542 | | SEQ ID NO: 2544 | |
| 54 | | | SEQ ID NO: 2542 | | SEQ ID NO: 2544 | SEQ ID NO: 2545 |
| 55 | | | SEQ ID NO: 2542 | | | SEQ ID NO: 2545 |
| 56 | | | SEQ ID NO: 2542 | SEQ ID NO: 2543 | | SEQ ID NO: 2545 |
| 57 | SEQ ID NO: 2541 | | SEQ ID NO: 2542 | SEQ ID NO: 2543 | | |
| 58 | SEQ ID NO: 2541 | | SEQ ID NO: 2542 | SEQ ID NO: 2543 | SEQ ID NO: 2544 | |
| 59 | SEQ ID NO: 2541 | | SEQ ID NO: 2542 | SEQ ID NO: 2543 | SEQ ID NO: 2544 | SEQ ID NO: 2545 |
| 60 | SEQ ID NO: 2541 | | SEQ ID NO: 2542 | | SEQ ID NO: 2544 | |
| 61 | SEQ ID NO: 2541 | | SEQ ID NO: 2542 | | SEQ ID NO: 2544 | SEQ ID NO: 2545 |
| 62 | SEQ ID NO: 2541 | | SEQ ID NO: 2542 | | | SEQ ID NO: 2545 |
| 63 | SEQ ID NO: 2541 | | SEQ ID NO: 2542 | SEQ ID NO: 2543 | | SEQ ID NO: 2545 |
| 64 | SEQ ID NO: 2548 | | | | | |
| 65 | SEQ ID NO: 2548 | KVS | | | | |
| 66 | SEQ ID NO: 2548 | KVS | SEQ ID NO: 2549 | | | |
| 67 | | KVS | | | | |
| 68 | | KVS | SEQ ID NO: 2549 | | | |
| 69 | | | SEQ ID NO: 2549 | | | |
| 70 | SEQ ID NO: 2548 | | SEQ ID NO: 2549 | | | |
| 71 | | | | SEQ ID NO: 2550 | | |
| 72 | | | | SEQ ID NO: 2550 | SEQ ID NO: 2551 | |
| 73 | | | | SEQ ID NO: 2550 | SEQ ID NO: 2551 | SEQ ID NO: 2552 |
| 74 | | | | | SEQ ID NO: 2551 | |
| 75 | | | | | SEQ ID NO: 2551 | SEQ ID NO: 2552 |
| 76 | | | | | | SEQ ID NO: 2552 |
| 77 | | | | SEQ ID NO: 2550 | | SEQ ID NO: 2552 |
| 78 | SEQ ID NO: 2548 | | | SEQ ID NO: 2550 | | |
| 79 | SEQ ID NO: 2548 | | | SEQ ID NO: 2550 | SEQ ID NO: 2551 | |
| 80 | SEQ ID NO: 2548 | | | SEQ ID NO: 2550 | SEQ ID NO: 2551 | SEQ ID NO: 2552 |
| 81 | SEQ ID NO: 2548 | | | | SEQ ID NO: 2551 | |
| 82 | SEQ ID NO: 2548 | | | | SEQ ID NO: 2551 | SEQ ID NO: 2552 |
| 83 | SEQ ID NO: 2548 | | | | | SEQ ID NO: 2552 |
| 84 | SEQ ID NO: 2548 | | | SEQ ID NO: 2550 | | SEQ ID NO: 2552 |
| 85 | SEQ ID NO: 2548 | KVS | | SEQ ID NO: 2550 | | |
| 86 | SEQ ID NO: 2548 | KVS | | SEQ ID NO: 2550 | SEQ ID NO: 2551 | |
| 87 | SEQ ID NO: 2548 | KVS | | SEQ ID NO: 2550 | SEQ ID NO: 2551 | SEQ ID NO: 2552 |
| 88 | SEQ ID NO: 2548 | KVS | | | SEQ ID NO: 2551 | |
| 89 | SEQ ID NO: 2548 | KVS | | | SEQ ID NO: 2551 | SEQ ID NO: 2552 |
| 90 | SEQ ID NO: 2548 | KVS | | | | SEQ ID NO: 2552 |
| 91 | SEQ ID NO: 2548 | KVS | | SEQ ID NO: 2550 | | SEQ ID NO: 2552 |
| 92 | SEQ ID NO: 2548 | KVS | SEQ ID NO: 2549 | SEQ ID NO: 2550 | | |
| 93 | SEQ ID NO: 2548 | KVS | SEQ ID NO: 2549 | SEQ ID NO: 2550 | SEQ ID NO: 2551 | |
| 94 | SEQ ID NO: 2548 | KVS | SEQ ID NO: 2549 | SEQ ID NO: 2550 | SEQ ID NO: 2551 | SEQ ID NO: 2552 |
| 95 | SEQ ID NO: 2548 | KVS | SEQ ID NO: 2549 | | SEQ ID NO: 2551 | |
| 96 | SEQ ID NO: 2548 | KVS | SEQ ID NO: 2549 | | SEQ ID NO: 2551 | SEQ ID NO: 2552 |
| 97 | SEQ ID NO: 2548 | KVS | SEQ ID NO: 2549 | | | SEQ ID NO: 2552 |
| 98 | SEQ ID NO: 2548 | KVS | SEQ ID NO: 2549 | SEQ ID NO: 2550 | | SEQ ID NO: 2552 |
| 99 | | KVS | | SEQ ID NO: 2550 | | |
| 100 | | KVS | | SEQ ID NO: 2550 | SEQ ID NO: 2551 | |
| 101 | | KVS | | SEQ ID NO: 2550 | SEQ ID NO: 2551 | SEQ ID NO: 2552 |
| 102 | | KVS | | | SEQ ID NO: 2551 | |
| 103 | | KVS | | | SEQ ID NO: 2551 | SEQ ID NO: 2552 |
| 104 | | KVS | | | | SEQ ID NO: 2552 |
| 105 | | KVS | | SEQ ID NO: 2550 | | SEQ ID NO: 2552 |
| 106 | | KVS | SEQ ID NO: 2549 | SEQ ID NO: 2550 | | |
| 107 | | KVS | SEQ ID NO: 2549 | SEQ ID NO: 2550 | SEQ ID NO: 2551 | |
| 108 | | KVS | SEQ ID NO: 2549 | SEQ ID NO: 2550 | SEQ ID NO: 2551 | SEQ ID NO: 2552 |
| 109 | | KVS | SEQ ID NO: 2549 | | SEQ ID NO: 2551 | |
| 110 | | KVS | SEQ ID NO: 2549 | | SEQ ID NO: 2551 | SEQ ID NO: 2552 |
| 111 | | KVS | SEQ ID NO: 2549 | | | SEQ ID NO: 2552 |
| 112 | | KVS | SEQ ID NO: 2549 | SEQ ID NO: 2550 | | SEQ ID NO: 2552 |
| 113 | | | SEQ ID NO: 2549 | SEQ ID NO: 2550 | | |
| 114 | | | SEQ ID NO: 2549 | SEQ ID NO: 2550 | SEQ ID NO: 2551 | |
| 115 | | | SEQ ID NO: 2549 | SEQ ID NO: 2550 | SEQ ID NO: 2551 | SEQ ID NO: 2552 |
| 116 | | | SEQ ID NO: 2549 | | SEQ ID NO: 2551 | |
| 117 | | | SEQ ID NO: 2549 | | SEQ ID NO: 2551 | SEQ ID NO: 2552 |
| 118 | | | SEQ ID NO: 2549 | | | SEQ ID NO: 2552 |
| 119 | | | SEQ ID NO: 2549 | SEQ ID NO: 2550 | | SEQ ID NO: 2552 |
| 120 | SEQ ID NO: 2548 | | SEQ ID NO: 2549 | SEQ ID NO: 2550 | | |
| 121 | SEQ ID NO: 2548 | | SEQ ID NO: 2549 | SEQ ID NO: 2550 | SEQ ID NO: 2551 | |
| 122 | SEQ ID NO: 2548 | | SEQ ID NO: 2549 | SEQ ID NO: 2550 | SEQ ID NO: 2551 | SEQ ID NO: 2552 |
| 123 | SEQ ID NO: 2548 | | SEQ ID NO: 2549 | | SEQ ID NO: 2551 | |
| 124 | SEQ ID NO: 2548 | | SEQ ID NO: 2549 | | SEQ ID NO: 2551 | SEQ ID NO: 2552 |
| 125 | SEQ ID NO: 2548 | | SEQ ID NO: 2549 | | | SEQ ID NO: 2552 |
| 126 | SEQ ID NO: 2548 | | SEQ ID NO: 2549 | SEQ ID NO: 2550 | | SEQ ID NO: 2552 |
| 127 | SEQ ID NO: 2555 | | | | | |
| 128 | SEQ ID NO: 2555 | KVS | | | | |
| 129 | SEQ ID NO: 2555 | KVS | SEQ ID NO: 2556 | | | |
| 130 | | KVS | | | | |
| 131 | | KVS | SEQ ID NO: 2556 | | | |
| 132 | | | SEQ ID NO: 2556 | | | |
| 133 | SEQ ID NO: 2555 | | SEQ ID NO: 2556 | | | |
| 134 | | | | SEQ ID NO: 2557 | | |
| 135 | | | | SEQ ID NO: 2557 | SEQ ID NO: 2558 | |
| 136 | | | | SEQ ID NO: 2557 | SEQ ID NO: 2558 | SEQ ID NO: 2559 |
| 137 | | | | | SEQ ID NO: 2558 | |
| 138 | | | | | SEQ ID NO: 2558 | SEQ ID NO: 2559 |
| 139 | | | | | | SEQ ID NO: 2559 |
| 140 | | | | SEQ ID NO: 2557 | | SEQ ID NO: 2559 |
| 141 | SEQ ID NO: 2555 | | | SEQ ID NO: 2557 | | |
| 142 | SEQ ID NO: 2555 | | | SEQ ID NO: 2557 | SEQ ID NO: 2558 | |
| 143 | SEQ ID NO: 2555 | | | SEQ ID NO: 2557 | SEQ ID NO: 2558 | SEQ ID NO: 2559 |
| 144 | SEQ ID NO: 2555 | | | | SEQ ID NO: 2558 | |
| 145 | SEQ ID NO: 2555 | | | | SEQ ID NO: 2558 | SEQ ID NO: 2559 |
| 146 | SEQ ID NO: 2555 | | | | | SEQ ID NO: 2559 |
| 147 | SEQ ID NO: 2555 | | | SEQ ID NO: 2557 | | SEQ ID NO: 2559 |
| 148 | SEQ ID NO: 2555 | KVS | | SEQ ID NO: 2557 | | |
| 149 | SEQ ID NO: 2555 | KVS | | SEQ ID NO: 2557 | SEQ ID NO: 2558 | |
| 150 | SEQ ID NO: 2555 | KVS | | SEQ ID NO: 2557 | SEQ ID NO: 2558 | SEQ ID NO: 2559 |
| 151 | SEQ ID NO: 2555 | KVS | | | SEQ ID NO: 2558 | |
| 152 | SEQ ID NO: 2555 | KVS | | | SEQ ID NO: 2558 | SEQ ID NO: 2559 |
| 153 | SEQ ID NO: 2555 | KVS | | | | SEQ ID NO: 2559 |
| 154 | SEQ ID NO: 2555 | KVS | | SEQ ID NO: 2557 | | SEQ ID NO: 2559 |
| 155 | SEQ ID NO: 2555 | KVS | SEQ ID NO: 2556 | SEQ ID NO: 2557 | | |
| 156 | SEQ ID NO: 2555 | KVS | SEQ ID NO: 2556 | SEQ ID NO: 2557 | SEQ ID NO: 2558 | |
| 157 | SEQ ID NO: 2555 | KVS | SEQ ID NO: 2556 | SEQ ID NO: 2557 | SEQ ID NO: 2558 | SEQ ID NO: 2559 |
| 158 | SEQ ID NO: 2555 | KVS | SEQ ID NO: 2556 | | SEQ ID NO: 2558 | |
| 159 | SEQ ID NO: 2555 | KVS | SEQ ID NO: 2556 | | SEQ ID NO: 2558 | SEQ ID NO: 2559 |
| 160 | SEQ ID NO: 2555 | KVS | SEQ ID NO: 2556 | SEQ ID NO: 2557 | | SEQ ID NO: 2559 |
| 161 | SEQ ID NO: 2555 | KVS | SEQ ID NO: 2556 | | | SEQ ID NO: 2559 |
| 162 | | KVS | | SEQ ID NO: 2557 | | |
| 163 | | KVS | | SEQ ID NO: 2557 | SEQ ID NO: 2558 | |
| 164 | | KVS | | SEQ ID NO: 2557 | SEQ ID NO: 2558 | SEQ ID NO: 2559 |
| 165 | | KVS | | | SEQ ID NO: 2558 | |
| 166 | | KVS | | | SEQ ID NO: 2558 | SEQ ID NO: 2559 |
| 167 | | KVS | | | | SEQ ID NO: 2559 |
| 168 | | KVS | | SEQ ID NO: 2557 | | SEQ ID NO: 2559 |
| 169 | | KVS | SEQ ID NO: 2556 | SEQ ID NO: 2557 | | |
| 170 | | KVS | SEQ ID NO: 2556 | SEQ ID NO: 2557 | SEQ ID NO: 2558 | |
| 171 | | KVS | SEQ ID NO: 2556 | SEQ ID NO: 2557 | SEQ ID NO: 2558 | SEQ ID NO: 2559 |
| 172 | | KVS | SEQ ID NO: 2556 | | SEQ ID NO: 2558 | |
| 173 | | KVS | SEQ ID NO: 2556 | | SEQ ID NO: 2558 | SEQ ID NO: 2559 |
| 174 | | KVS | SEQ ID NO: 2556 | | | SEQ ID NO: 2559 |
| 175 | | KVS | SEQ ID NO: 2556 | SEQ ID NO: 2557 | | SEQ ID NO: 2559 |
| 176 | | | SEQ ID NO: 2556 | SEQ ID NO: 2557 | | |
| 177 | | | SEQ ID NO: 2556 | SEQ ID NO: 2557 | SEQ ID NO: 2558 | |
| 178 | | | SEQ ID NO: 2556 | SEQ ID NO: 2557 | SEQ ID NO: 2558 | SEQ ID NO: 2559 |
| 179 | | | SEQ ID NO: 2556 | | SEQ ID NO: 2558 | |
| 180 | | | SEQ ID NO: 2556 | | SEQ ID NO: 2558 | SEQ ID NO: 2559 |
| 181 | | | SEQ ID NO: 2556 | | | SEQ ID NO: 2559 |
| 182 | | | SEQ ID NO: 2556 | SEQ ID NO: 2557 | | SEQ ID NO: 2559 |
| 183 | SEQ ID NO: 2555 | | SEQ ID NO: 2556 | SEQ ID NO: 2557 | | |
| 184 | SEQ ID NO: 2555 | | SEQ ID NO: 2556 | SEQ ID NO: 2557 | SEQ ID NO: 2558 | |
| 185 | SEQ ID NO: 2555 | | SEQ ID NO: 2556 | SEQ ID NO: 2557 | SEQ ID NO: 2558 | SEQ ID NO: 2559 |
| 186 | SEQ ID NO: 2555 | | SEQ ID NO: 2556 | | SEQ ID NO: 2558 | |
| 187 | SEQ ID NO: 2555 | | SEQ ID NO: 2556 | | SEQ ID NO: 2558 | SEQ ID NO: 2559 |
| 188 | SEQ ID NO: 2555 | | SEQ ID NO: 2556 | | | SEQ ID NO: 2559 |
| 189 | SEQ ID NO: 2555 | | SEQ ID NO: 2556 | SEQ ID NO: 2557 | | SEQ ID NO: 2559 |
| 190 | SEQ ID NO: 2562 | | | | | |
| 191 | SEQ ID NO: 2562 | KVS | | | | |
| 192 | SEQ ID NO: 2562 | KVS | SEQ ID NO: 25 | | | |
| 193 | | KVS | | | | |
| 194 | | KVS | SEQ ID NO: 25 | | | |
| 195 | | | SEQ ID NO: 25 | | | |
| 196 | SEQ ID NO: 2562 | | SEQ ID NO: 25 | | | |
| 197 | | | | SEQ ID NO: 2564 | | |
| 198 | | | | SEQ ID NO: 2564 | SEQ ID NO: 2565 | |
| 199 | | | | SEQ ID NO: 2564 | SEQ ID NO: 2565 | SEQ ID NO: 2566 |
| 200 | | | | | SEQ ID NO: 2565 | |
| 201 | | | | | SEQ ID NO: 2565 | SEQ ID NO: 2566 |
| 202 | | | | | | SEQ ID NO: 2566 |
| 203 | | | | SEQ ID NO: 2564 | | SEQ ID NO: 2566 |
| 204 | SEQ ID NO: 2562 | | | SEQ ID NO: 2564 | | |
| 205 | SEQ ID NO: 2562 | | | SEQ ID NO: 2564 | SEQ ID NO: 2565 | |
| 206 | SEQ ID NO: 2562 | | | SEQ ID NO: 2564 | SEQ ID NO: 2565 | SEQ ID NO: 2566 |
| 207 | SEQ ID NO: 2562 | | | | SEQ ID NO: 2565 | |
| 208 | SEQ ID NO: 2562 | | | | SEQ ID NO: 2565 | SEQ ID NO: 2566 |
| 209 | SEQ ID NO: 2562 | | | | | SEQ ID NO: 2566 |
| 210 | SEQ ID NO: 2562 | | | SEQ ID NO: 2564 | | SEQ ID NO: 2566 |
| 211 | SEQ ID NO: 2562 | KVS | | SEQ ID NO: 2564 | | |
| 212 | SEQ ID NO: 2562 | KVS | | SEQ ID NO: 2564 | SEQ ID NO: 2565 | |
| 213 | SEQ ID NO: 2562 | KVS | | SEQ ID NO: 2564 | SEQ ID NO: 2565 | SEQ ID NO: 2566 |
| 214 | SEQ ID NO: 2562 | KVS | | | SEQ ID NO: 2565 | |
| 215 | SEQ ID NO: 2562 | KVS | | | SEQ ID NO: 2565 | SEQ ID NO: 2566 |
| 216 | SEQ ID NO: 2562 | KVS | | | | SEQ ID NO: 2566 |
| 217 | SEQ ID NO: 2562 | KVS | | SEQ ID NO: 2564 | | SEQ ID NO: 2566 |
| 218 | SEQ ID NO: 2562 | KVS | SEQ ID NO: 25 | SEQ ID NO: 2564 | | |
| 219 | SEQ ID NO: 2562 | KVS | SEQ ID NO: 25 | SEQ ID NO: 2564 | SEQ ID NO: 2565 | |
| 220 | SEQ ID NO: 2562 | KVS | SEQ ID NO: 25 | SEQ ID NO: 2564 | SEQ ID NO: 2565 | SEQ ID NO: 2566 |
| 221 | SEQ ID NO: 2562 | KVS | SEQ ID NO: 25 | | SEQ ID NO: 2565 | |
| 222 | SEQ ID NO: 2562 | KVS | SEQ ID NO: 25 | | SEQ ID NO: 2565 | SEQ ID NO: 2566 |
| 223 | SEQ ID NO: 2562 | KVS | SEQ ID NO: 25 | SEQ ID NO: 2564 | | SEQ ID NO: 2566 |
| 224 | SEQ ID NO: 2562 | KVS | SEQ ID NO: 25 | | | SEQ ID NO: 2566 |
| 225 | | KVS | | SEQ ID NO: 2564 | | |
| 226 | | KVS | | SEQ ID NO: 2564 | SEQ ID NO: 2565 | |
| 227 | | KVS | | SEQ ID NO: 2564 | SEQ ID NO: 2565 | SEQ ID NO: 2566 |
| 228 | | KVS | | | SEQ ID NO: 2565 | |
| 229 | | KVS | | | SEQ ID NO: 2565 | SEQ ID NO: 2566 |
| 230 | | KVS | | | | SEQ ID NO: 2566 |
| 231 | | KVS | | SEQ ID NO: 2564 | | SEQ ID NO: 2566 |
| 232 | | KVS | SEQ ID NO: 25 | SEQ ID NO: 2564 | | |
| 233 | | KVS | SEQ ID NO: 25 | SEQ ID NO: 2564 | SEQ ID NO: 2565 | |
| 234 | | KVS | SEQ ID NO: 25 | SEQ ID NO: 2564 | SEQ ID NO: 2565 | SEQ ID NO: 2566 |
| 235 | | KVS | SEQ ID NO: 25 | | SEQ ID NO: 2565 | |
| 236 | | KVS | SEQ ID NO: 25 | | SEQ ID NO: 2565 | SEQ ID NO: 2566 |
| 237 | | KVS | SEQ ID NO: 25 | | | SEQ ID NO: 2566 |
| 238 | | KVS | SEQ ID NO: 25 | SEQ ID NO: 2564 | | SEQ ID NO: 2566 |
| 239 | | | SEQ ID NO: 25 | SEQ ID NO: 2564 | | |
| 240 | | | SEQ ID NO: 25 | SEQ ID NO: 2564 | SEQ ID NO: 2565 | |
| 241 | | | SEQ ID NO: 25 | SEQ ID NO: 2564 | SEQ ID NO: 2565 | SEQ ID NO: 2566 |
| 242 | | | SEQ ID NO: 25 | | SEQ ID NO: 2565 | |
| 243 | | | SEQ ID NO: 25 | | SEQ ID NO: 2565 | SEQ ID NO: 2566 |
| 244 | | | SEQ ID NO: 25 | | | SEQ ID NO: 2566 |
| 245 | | | SEQ ID NO: 25 | SEQ ID NO: 2564 | | SEQ ID NO: 2566 |
| 246 | SEQ ID NO: 2562 | | SEQ ID NO: 25 | SEQ ID NO: 2564 | | |
| 247 | SEQ ID NO: 2562 | | SEQ ID NO: 25 | SEQ ID NO: 2564 | SEQ ID NO: 2565 | |
| 248 | SEQ ID NO: 2562 | | SEQ ID NO: 25 | SEQ ID NO: 2564 | SEQ ID NO: 2565 | SEQ ID NO: 2566 |
| 249 | SEQ ID NO: 2562 | | SEQ ID NO: 25 | | SEQ ID NO: 2565 | |
| 250 | SEQ ID NO: 2562 | | SEQ ID NO: 25 | | SEQ ID NO: 2565 | SEQ ID NO: 2566 |
| 251 | SEQ ID NO: 2562 | | SEQ ID NO: 25 | | | SEQ ID NO: 2566 |
| 252 | SEQ ID NO: 2562 | | SEQ ID NO: 25 | SEQ ID NO: 2564 | | SEQ ID NO: 2566 |
| 253 | SEQ ID NO: 2569 | | | | | |
| 254 | SEQ ID NO: 2569 | KVS | | | | |
| 255 | SEQ ID NO: 2569 | KVS | SEQ ID NO: 2570 | | | |
| 256 | | KVS | | | | |
| 257 | | KVS | SEQ ID NO: 2570 | | | |
| 258 | | | SEQ ID NO: 2570 | | | |
| 259 | SEQ ID NO: 2569 | | SEQ ID NO: 2570 | | | |
| 260 | | | | SEQ ID NO: 2571 | | |
| 261 | | | | SEQ ID NO: 2571 | SEQ ID NO: 2572 | |
| 262 | | | | SEQ ID NO: 2571 | SEQ ID NO: 2572 | SEQ ID NO: 2573 |
| 263 | | | | | SEQ ID NO: 2572 | |
| 264 | | | | | SEQ ID NO: 2572 | SEQ ID NO: 2573 |
| 265 | | | | | | SEQ ID NO: 2573 |
| 266 | | | | SEQ ID NO: 2571 | | SEQ ID NO: 2573 |
| 267 | SEQ ID NO: 2569 | | | SEQ ID NO: 2571 | | |
| 268 | SEQ ID NO: 2569 | | | SEQ ID NO: 2571 | SEQ ID NO: 2572 | |
| 269 | SEQ ID NO: 2569 | | | SEQ ID NO: 2571 | SEQ ID NO: 2572 | SEQ ID NO: 2573 |
| 270 | SEQ ID NO: 2569 | | | | SEQ ID NO: 2572 | |
| 271 | SEQ ID NO: 2569 | | | | SEQ ID NO: 2572 | SEQ ID NO: 2573 |
| 272 | SEQ ID NO: 2569 | | | | | SEQ ID NO: 2573 |
| 273 | SEQ ID NO: 2569 | | | SEQ ID NO: 2571 | | SEQ ID NO: 2573 |
| 274 | SEQ ID NO: 2569 | KVS | | SEQ ID NO: 2571 | | |
| 275 | SEQ ID NO: 2569 | KVS | | SEQ ID NO: 2571 | SEQ ID NO: 2572 | |
| 276 | SEQ ID NO: 2569 | KVS | | SEQ ID NO: 2571 | SEQ ID NO: 2572 | SEQ ID NO: 2573 |
| 277 | SEQ ID NO: 2569 | KVS | | | SEQ ID NO: 2572 | |
| 278 | SEQ ID NO: 2569 | KVS | | | SEQ ID NO: 2572 | SEQ ID NO: 2573 |
| 279 | SEQ ID NO: 2569 | KVS | | | | SEQ ID NO: 2573 |
| 280 | SEQ ID NO: 2569 | KVS | | SEQ ID NO: 2571 | | SEQ ID NO: 2573 |
| 281 | SEQ ID NO: 2569 | KVS | SEQ ID NO: 2570 | SEQ ID NO: 2571 | | |
| 282 | SEQ ID NO: 2569 | KVS | SEQ ID NO: 2570 | SEQ ID NO: 2571 | SEQ ID NO: 2572 | |
| 283 | SEQ ID NO: 2569 | KVS | SEQ ID NO: 2570 | SEQ ID NO: 2571 | SEQ ID NO: 2572 | SEQ ID NO: 2573 |
| 284 | SEQ ID NO: 2569 | KVS | SEQ ID NO: 2570 | | SEQ ID NO: 2572 | |
| 285 | SEQ ID NO: 2569 | KVS | SEQ ID NO: 2570 | | SEQ ID NO: 2572 | SEQ ID NO: 2573 |
| 286 | SEQ ID NO: 2569 | KVS | SEQ ID NO: 2570 | SEQ ID NO: 2571 | | SEQ ID NO: 2573 |
| 287 | SEQ ID NO: 2569 | KVS | SEQ ID NO: 2570 | | | SEQ ID NO: 2573 |
| 288 | | KVS | | SEQ ID NO: 2571 | | |
| 289 | | KVS | | SEQ ID NO: 2571 | SEQ ID NO: 2572 | |
| 290 | | KVS | | SEQ ID NO: 2571 | SEQ ID NO: 2572 | SEQ ID NO: 2573 |
| 291 | | KVS | | | SEQ ID NO: 2572 | |
| 292 | | KVS | | | SEQ ID NO: 2572 | SEQ ID NO: 2573 |
| 293 | | KVS | | | | SEQ ID NO: 2573 |
| 294 | | KVS | | SEQ ID NO: 2571 | | SEQ ID NO: 2573 |
| 295 | | KVS | SEQ ID NO: 2570 | SEQ ID NO: 2571 | | |
| 296 | | KVS | SEQ ID NO: 2570 | SEQ ID NO: 2571 | SEQ ID NO: 2572 | |
| 297 | | KVS | SEQ ID NO: 2570 | SEQ ID NO: 2571 | SEQ ID NO: 2572 | SEQ ID NO: 2573 |
| 298 | | KVS | SEQ ID NO: 2570 | | SEQ ID NO: 2572 | |
| 299 | | KVS | SEQ ID NO: 2570 | | SEQ ID NO: 2572 | SEQ ID NO: 2573 |
| 300 | | KVS | SEQ ID NO: 2570 | | | SEQ ID NO: 2573 |
| 301 | | KVS | SEQ ID NO: 2570 | SEQ ID NO: 2571 | | SEQ ID NO: 2573 |
| 302 | | | SEQ ID NO: 2570 | SEQ ID NO: 2571 | | |
| 303 | | | SEQ ID NO: 2570 | SEQ ID NO: 2571 | SEQ ID NO: 2572 | |
| 304 | | | SEQ ID NO: 2570 | SEQ ID NO: 2571 | SEQ ID NO: 2572 | SEQ ID NO: 2573 |
| 305 | | | SEQ ID NO: 2570 | | SEQ ID NO: 2572 | |
| 306 | | | SEQ ID NO: 2570 | | SEQ ID NO: 2572 | SEQ ID NO: 2573 |
| 307 | | | SEQ ID NO: 2570 | | | SEQ ID NO: 2573 |
| 308 | | | SEQ ID NO: 2570 | SEQ ID NO: 2571 | | SEQ ID NO: 2573 |
| 309 | SEQ ID NO: 2569 | | SEQ ID NO: 2570 | SEQ ID NO: 2571 | | |
| 310 | SEQ ID NO: 2569 | | SEQ ID NO: 2570 | SEQ ID NO: 2571 | SEQ ID NO: 2572 | |
| 311 | SEQ ID NO: 2569 | | SEQ ID NO: 2570 | SEQ ID NO: 2571 | SEQ ID NO: 2572 | SEQ ID NO: 2573 |
| 312 | SEQ ID NO: 2569 | | SEQ ID NO: 2570 | | SEQ ID NO: 2572 | |
| 313 | SEQ ID NO: 2569 | | SEQ ID NO: 2570 | | SEQ ID NO: 2572 | SEQ ID NO: 2573 |
| 314 | SEQ ID NO: 2569 | | SEQ ID NO: 2570 | | | SEQ ID NO: 2573 |
| 315 | SEQ ID NO: 2569 | | SEQ ID NO: 2570 | SEQ ID NO: 2571 | | SEQ ID NO: 2573 |
| 316 | SEQ ID NO: 2576 | | | | | |
| 317 | SEQ ID NO: 2576 | WAS | | | | |
| 318 | SEQ ID NO: 2576 | WAS | SEQ ID NO: 2577 | | | |
| 319 | | WAS | | | | |
| 320 | | WAS | SEQ ID NO: 2577 | | | |
| 321 | | | SEQ ID NO: 2577 | | | |
| 322 | SEQ ID NO: 2576 | | SEQ ID NO: 2577 | | | |
| 323 | | | | SEQ ID NO: 2578 | | |
| 324 | | | | SEQ ID NO: 2578 | SEQ ID NO: 2579 | |
| 325 | | | | SEQ ID NO: 2578 | SEQ ID NO: 2579 | SEQ ID NO: 2580 |
| 326 | | | | | SEQ ID NO: 2579 | |
| 327 | | | | | SEQ ID NO: 2579 | SEQ ID NO: 2580 |
| 328 | | | | | | SEQ ID NO: 2580 |
| 329 | | | | SEQ ID NO: 2578 | | SEQ ID NO: 2580 |
| 330 | SEQ ID NO: 2576 | | | SEQ ID NO: 2578 | | |
| 331 | SEQ ID NO: 2576 | | | SEQ ID NO: 2578 | SEQ ID NO: 2579 | |
| 332 | SEQ ID NO: 2576 | | | SEQ ID NO: 2578 | SEQ ID NO: 2579 | SEQ ID NO: 2580 |
| 333 | SEQ ID NO: 2576 | | | | SEQ ID NO: 2579 | |
| 334 | SEQ ID NO: 2576 | | | | SEQ ID NO: 2579 | SEQ ID NO: 2580 |
| 335 | SEQ ID NO: 2576 | | | | | SEQ ID NO: 2580 |
| 336 | SEQ ID NO: 2576 | | | SEQ ID NO: 2578 | | SEQ ID NO: 2580 |
| 337 | SEQ ID NO: 2576 | WAS | | SEQ ID NO: 2578 | | |
| 338 | SEQ ID NO: 2576 | WAS | | SEQ ID NO: 2578 | SEQ ID NO: 2579 | |
| 339 | SEQ ID NO: 2576 | WAS | | SEQ ID NO: 2578 | SEQ ID NO: 2579 | SEQ ID NO: 2580 |
| 340 | SEQ ID NO: 2576 | WAS | | | SEQ ID NO: 2579 | |
| 341 | SEQ ID NO: 2576 | WAS | | | SEQ ID NO: 2579 | SEQ ID NO: 2580 |
| 342 | SEQ ID NO: 2576 | WAS | | | | SEQ ID NO: 2580 |
| 343 | SEQ ID NO: 2576 | WAS | | SEQ ID NO: 2578 | | SEQ ID NO: 2580 |
| 344 | SEQ ID NO: 2576 | WAS | SEQ ID NO: 2577 | SEQ ID NO: 2578 | | |
| 345 | SEQ ID NO: 2576 | WAS | SEQ ID NO: 2577 | SEQ ID NO: 2578 | SEQ ID NO: 2579 | |
| 346 | SEQ ID NO: 2576 | WAS | SEQ ID NO: 2577 | SEQ ID NO: 2578 | SEQ ID NO: 2579 | SEQ ID NO: 2580 |
| 347 | SEQ ID NO: 2576 | WAS | SEQ ID NO: 2577 | | SEQ ID NO: 2579 | |
| 348 | SEQ ID NO: 2576 | WAS | SEQ ID NO: 2577 | | SEQ ID NO: 2579 | SEQ ID NO: 2580 |
| 349 | SEQ ID NO: 2576 | WAS | SEQ ID NO: 2577 | SEQ ID NO: 2578 | | SEQ ID NO: 2580 |
| 350 | SEQ ID NO: 2576 | WAS | SEQ ID NO: 2577 | | | SEQ ID NO: 2580 |
| 351 | | WAS | | SEQ ID NO: 2578 | | |
| 352 | | WAS | | SEQ ID NO: 2578 | SEQ ID NO: 2579 | |
| 353 | | WAS | | SEQ ID NO: 2578 | SEQ ID NO: 2579 | SEQ ID NO: 2580 |
| 354 | | WAS | | | SEQ ID NO: 2579 | |
| 355 | | WAS | | | SEQ ID NO: 2579 | SEQ ID NO: 2580 |
| 356 | | WAS | | | | SEQ ID NO: 2580 |
| 357 | | WAS | | SEQ ID NO: 2578 | | SEQ ID NO: 2580 |
| 358 | | WAS | SEQ ID NO: 2577 | SEQ ID NO: 2578 | | |
| 359 | | WAS | SEQ ID NO: 2577 | SEQ ID NO: 2578 | SEQ ID NO: 2579 | |
| 360 | | WAS | SEQ ID NO: 2577 | SEQ ID NO: 2578 | SEQ ID NO: 2579 | SEQ ID NO: 2580 |
| 361 | | WAS | SEQ ID NO: 2577 | | SEQ ID NO: 2579 | |
| 362 | | WAS | SEQ ID NO: 2577 | | SEQ ID NO: 2579 | SEQ ID NO: 2580 |
| 363 | | WAS | SEQ ID NO: 2577 | | | SEQ ID NO: 2580 |
| 364 | | WAS | SEQ ID NO: 2577 | SEQ ID NO: 2578 | | SEQ ID NO: 2580 |
| 365 | | | SEQ ID NO: 2577 | SEQ ID NO: 2578 | | |
| 366 | | | SEQ ID NO: 2577 | SEQ ID NO: 2578 | SEQ ID NO: 2579 | |
| 367 | | | SEQ ID NO: 2577 | SEQ ID NO: 2578 | SEQ ID NO: 2579 | SEQ ID NO: 2580 |
| 368 | | | SEQ ID NO: 2577 | | SEQ ID NO: 2579 | |
| 369 | | | SEQ ID NO: 2577 | | SEQ ID NO: 2579 | SEQ ID NO: 2580 |
| 370 | | | SEQ ID NO: 2577 | | | SEQ ID NO: 2580 |
| 371 | | | SEQ ID NO: 2577 | SEQ ID NO: 2578 | | SEQ ID NO: 2580 |
| 372 | SEQ ID NO: 2576 | | SEQ ID NO: 2577 | SEQ ID NO: 2578 | | |
| 373 | SEQ ID NO: 2576 | | SEQ ID NO: 2577 | SEQ ID NO: 2578 | SEQ ID NO: 2579 | |
| 374 | SEQ ID NO: 2576 | | SEQ ID NO: 2577 | SEQ ID NO: 2578 | SEQ ID NO: 2579 | SEQ ID NO: 2580 |
| 375 | SEQ ID NO: 2576 | | SEQ ID NO: 2577 | | SEQ ID NO: 2579 | |
| 376 | SEQ ID NO: 2576 | | SEQ ID NO: 2577 | | SEQ ID NO: 2579 | SEQ ID NO: 2580 |
| 377 | SEQ ID NO: 2576 | | SEQ ID NO: 2577 | | | SEQ ID NO: 2580 |
| 378 | SEQ ID NO: 2576 | | SEQ ID NO: 2577 | SEQ ID NO: 2578 | | SEQ ID NO: 2580 |

TABLE I3

| | Light Chain HVR | | | Heavy Chain HVR | | |
|---|---|---|---|---|---|---|
| Antibody | L1 | L2 | L3 | H1 | H2 | H3 |
| 1 | SEQ ID NO: 2582 | | | | | |
| 2 | SEQ ID NO: 2582 | (I/V)KS | | | | |
| 3 | SEQ ID NO: 2582 | (I/V)KS | SEQ ID NO: 2583 | | | |
| 4 | | (I/V)KS | | | | |
| 5 | | (I/V)KS | SEQ ID NO: 2583 | | | |
| 6 | | | SEQ ID NO: 2583 | | | |
| 7 | SEQ ID NO: 2582 | | SEQ ID NO: 2583 | | | |
| 8 | SEQ ID NO: 2582 | | | SEQ ID NO: 2543 | | |
| 9 | SEQ ID NO: 2582 | | | SEQ ID NO: 2543 | SEQ ID NO: 2544 | |
| 10 | SEQ ID NO: 2582 | | | SEQ ID NO: 2543 | SEQ ID NO: 2544 | SEQ ID NO: 2545 |
| 11 | SEQ ID NO: 2582 | | | | SEQ ID NO: 2544 | |
| 12 | SEQ ID NO: 2582 | | | | SEQ ID NO: 2544 | SEQ ID NO: 2545 |
| 13 | SEQ ID NO: 2582 | | | | | SEQ ID NO: 2545 |
| 14 | SEQ ID NO: 2582 | | | SEQ ID NO: 2543 | | SEQ ID NO: 2545 |
| 15 | SEQ ID NO: 2582 | (I/V)KS | | SEQ ID NO: 2543 | | |
| 16 | SEQ ID NO: 2582 | (I/V)KS | | SEQ ID NO: 2543 | SEQ ID NO: 2544 | |
| 17 | SEQ ID NO: 2582 | (I/V)KS | | SEQ ID NO: 2543 | SEQ ID NO: 2544 | SEQ ID NO: 2545 |
| 18 | SEQ ID NO: 2582 | (I/V)KS | | | SEQ ID NO: 2544 | |
| 19 | SEQ ID NO: 2582 | (I/V)KS | | | SEQ ID NO: 2544 | SEQ ID NO: 2545 |
| 20 | SEQ ID NO: 2582 | (I/V)KS | | | | SEQ ID NO: 2545 |
| 21 | SEQ ID NO: 2582 | (I/V)KS | | SEQ ID NO: 2543 | | SEQ ID NO: 2545 |
| 22 | SEQ ID NO: 2582 | (I/V)KS | SEQ ID NO: 2583 | SEQ ID NO: 2543 | | |
| 23 | SEQ ID NO: 2582 | (I/V)KS | SEQ ID NO: 2583 | SEQ ID NO: 2543 | SEQ ID NO: 2544 | |
| 24 | SEQ ID NO: 2582 | (I/V)KS | SEQ ID NO: 2583 | SEQ ID NO: 2543 | SEQ ID NO: 2544 | SEQ ID NO: 2545 |
| 25 | SEQ ID NO: 2582 | (I/V)KS | SEQ ID NO: 2583 | | SEQ ID NO: 2544 | |
| 26 | SEQ ID NO: 2582 | (I/V)KS | SEQ ID NO: 2583 | | SEQ ID NO: 2544 | SEQ ID NO: 2545 |
| 27 | SEQ ID NO: 2582 | (I/V)KS | SEQ ID NO: 2583 | SEQ ID NO: 2543 | | SEQ ID NO: 2545 |
| 28 | SEQ ID NO: 2582 | (I/V)KS | SEQ ID NO: 2583 | | | SEQ ID NO: 2545 |
| 29 | | (I/V)KS | | SEQ ID NO: 2543 | | |
| 30 | | (I/V)KS | | SEQ ID NO: 2543 | SEQ ID NO: 2544 | |
| 31 | | (I/V)KS | | SEQ ID NO: 2543 | SEQ ID NO: 2544 | SEQ ID NO: 2545 |
| 32 | | (I/V)KS | | | SEQ ID NO: 2544 | |
| 33 | | (I/V)KS | | | SEQ ID NO: 2544 | SEQ ID NO: 2545 |
| 34 | | (I/V)KS | | | | SEQ ID NO: 2545 |
| 35 | | (I/V)KS | | SEQ ID NO: 2543 | | SEQ ID NO: 2545 |
| 36 | | (I/V)KS | SEQ ID NO: 2583 | SEQ ID NO: 2543 | | |
| 37 | | (I/V)KS | SEQ ID NO: 2583 | SEQ ID NO: 2543 | SEQ ID NO: 2544 | |
| 38 | | (I/V)KS | SEQ ID NO: 2583 | SEQ ID NO: 2543 | SEQ ID NO: 2544 | SEQ ID NO: 2545 |
| 39 | | (I/V)KS | SEQ ID NO: 2583 | | SEQ ID NO: 2544 | |
| 40 | | (I/V)KS | SEQ ID NO: 2583 | | SEQ ID NO: 2544 | SEQ ID NO: 2545 |
| 41 | | (I/V)KS | SEQ ID NO: 2583 | | | SEQ ID NO: 2545 |
| 42 | | (I/V)KS | SEQ ID NO: 2583 | SEQ ID NO: 2543 | | SEQ ID NO: 2545 |
| 43 | | | SEQ ID NO: 2583 | SEQ ID NO: 2543 | | |
| 44 | | | SEQ ID NO: 2583 | SEQ ID NO: 2543 | SEQ ID NO: 2544 | |
| 45 | | | SEQ ID NO: 2583 | SEQ ID NO: 2543 | SEQ ID NO: 2544 | SEQ ID NO: 2545 |
| 46 | | | SEQ ID NO: 2583 | | SEQ ID NO: 2544 | |
| 47 | | | SEQ ID NO: 2583 | | SEQ ID NO: 2544 | SEQ ID NO: 2545 |
| 48 | | | SEQ ID NO: 2583 | | | SEQ ID NO: 2545 |
| 49 | | | SEQ ID NO: 2583 | SEQ ID NO: 2543 | | SEQ ID NO: 2545 |
| 50 | SEQ ID NO: 2582 | | SEQ ID NO: 2583 | SEQ ID NO: 2543 | | |
| 51 | SEQ ID NO: 2582 | | SEQ ID NO: 2583 | SEQ ID NO: 2543 | SEQ ID NO: 2544 | |
| 52 | SEQ ID NO: 2582 | | SEQ ID NO: 2583 | SEQ ID NO: 2543 | SEQ ID NO: 2544 | SEQ ID NO: 2545 |
| 53 | SEQ ID NO: 2582 | | SEQ ID NO: 2583 | | SEQ ID NO: 2544 | |
| 54 | SEQ ID NO: 2582 | | SEQ ID NO: 2583 | | SEQ ID NO: 2544 | SEQ ID NO: 2545 |
| 55 | SEQ ID NO: 2582 | | SEQ ID NO: 2583 | | | SEQ ID NO: 2545 |
| 56 | SEQ ID NO: 2582 | | SEQ ID NO: 2583 | SEQ ID NO: 2543 | | SEQ ID NO: 2545 |
| 57 | SEQ ID NO: 2582 | | | SEQ ID NO: 2550 | | |
| 58 | SEQ ID NO: 2582 | | | SEQ ID NO: 2550 | SEQ ID NO: 2551 | |
| 59 | SEQ ID NO: 2582 | | | SEQ ID NO: 2550 | SEQ ID NO: 2551 | SEQ ID NO: 2552 |
| 60 | SEQ ID NO: 2582 | | | | SEQ ID NO: 2551 | |
| 61 | SEQ ID NO: 2582 | | | | SEQ ID NO: 2551 | SEQ ID NO: 2552 |
| 62 | SEQ ID NO: 2582 | | | | | SEQ ID NO: 2552 |
| 63 | SEQ ID NO: 2582 | | | SEQ ID NO: 2550 | | SEQ ID NO: 2552 |
| 64 | SEQ ID NO: 2582 | (I/V)KS | | SEQ ID NO: 2550 | | |
| 65 | SEQ ID NO: 2582 | (I/V)KS | | SEQ ID NO: 2550 | SEQ ID NO: 2551 | |
| 66 | SEQ ID NO: 2582 | (I/V)KS | | SEQ ID NO: 2550 | SEQ ID NO: 2551 | SEQ ID NO: 2552 |
| 67 | SEQ ID NO: 2582 | (I/V)KS | | | SEQ ID NO: 2551 | |
| 68 | SEQ ID NO: 2582 | (I/V)KS | | | SEQ ID NO: 2551 | SEQ ID NO: 2552 |
| 69 | SEQ ID NO: 2582 | (I/V)KS | | | | SEQ ID NO: 2552 |
| 70 | SEQ ID NO: 2582 | (I/V)KS | | SEQ ID NO: 2550 | | SEQ ID NO: 2552 |
| 71 | SEQ ID NO: 2582 | (I/V)KS | SEQ ID NO: 2583 | SEQ ID NO: 2550 | | |
| 72 | SEQ ID NO: 2582 | (I/V)KS | SEQ ID NO: 2583 | SEQ ID NO: 2550 | SEQ ID NO: 2551 | |
| 73 | SEQ ID NO: 2582 | (I/V)KS | SEQ ID NO: 2583 | SEQ ID NO: 2550 | SEQ ID NO: 2551 | SEQ ID NO: 2552 |
| 74 | SEQ ID NO: 2582 | (I/V)KS | SEQ ID NO: 2583 | | SEQ ID NO: 2551 | |
| 75 | SEQ ID NO: 2582 | (I/V)KS | SEQ ID NO: 2583 | | SEQ ID NO: 2551 | SEQ ID NO: 2552 |
| 76 | SEQ ID NO: 2582 | (I/V)KS | SEQ ID NO: 2583 | | | SEQ ID NO: 2552 |
| 77 | SEQ ID NO: 2582 | (I/V)KS | SEQ ID NO: 2583 | SEQ ID NO: 2550 | | SEQ ID NO: 2552 |
| 78 | | (I/V)KS | | SEQ ID NO: 2550 | | |
| 79 | | (I/V)KS | | SEQ ID NO: 2550 | SEQ ID NO: 2551 | |
| 80 | | (I/V)KS | | SEQ ID NO: 2550 | SEQ ID NO: 2551 | SEQ ID NO: 2552 |
| 81 | | (I/V)KS | | | SEQ ID NO: 2551 | |
| 82 | | (I/V)KS | | | SEQ ID NO: 2551 | SEQ ID NO: 2552 |
| 83 | | (I/V)KS | | | | SEQ ID NO: 2552 |
| 84 | | (I/V)KS | | SEQ ID NO: 2550 | | SEQ ID NO: 2552 |
| 85 | | (I/V)KS | SEQ ID NO: 2583 | SEQ ID NO: 2550 | | |
| 86 | | (I/V)KS | SEQ ID NO: 2583 | SEQ ID NO: 2550 | SEQ ID NO: 2551 | |
| 87 | | (I/V)KS | SEQ ID NO: 2583 | SEQ ID NO: 2550 | SEQ ID NO: 2551 | SEQ ID NO: 2552 |
| 88 | | (I/V)KS | SEQ ID NO: 2583 | | SEQ ID NO: 2551 | |
| 89 | | (I/V)KS | SEQ ID NO: 2583 | | SEQ ID NO: 2551 | SEQ ID NO: 2552 |
| 90 | | (I/V)KS | SEQ ID NO: 2583 | | | SEQ ID NO: 2552 |
| 91 | | (I/V)KS | SEQ ID NO: 2583 | SEQ ID NO: 2550 | | SEQ ID NO: 2552 |
| 92 | | | SEQ ID NO: 2583 | SEQ ID NO: 2550 | | |
| 93 | | | SEQ ID NO: 2583 | SEQ ID NO: 2550 | SEQ ID NO: 2551 | |
| 94 | | | SEQ ID NO: 2583 | SEQ ID NO: 2550 | SEQ ID NO: 2551 | SEQ ID NO: 2552 |
| 95 | | | SEQ ID NO: 2583 | | SEQ ID NO: 2551 | |
| 96 | | | SEQ ID NO: 2583 | | SEQ ID NO: 2551 | SEQ ID NO: 2552 |
| 97 | | | SEQ ID NO: 2583 | | | SEQ ID NO: 2552 |
| 98 | | | SEQ ID NO: 2583 | SEQ ID NO: 2550 | | SEQ ID NO: 2552 |
| 99 | SEQ ID NO: 2582 | | SEQ ID NO: 2583 | SEQ ID NO: 2550 | | |
| 100 | SEQ ID NO: 2582 | | SEQ ID NO: 2583 | SEQ ID NO: 2550 | SEQ ID NO: 2551 | |
| 101 | SEQ ID NO: 2582 | | SEQ ID NO: 2583 | SEQ ID NO: 2550 | SEQ ID NO: 2551 | SEQ ID NO: 2552 |
| 102 | SEQ ID NO: 2582 | | SEQ ID NO: 2583 | | SEQ ID NO: 2551 | |
| 103 | SEQ ID NO: 2582 | | SEQ ID NO: 2583 | | SEQ ID NO: 2551 | SEQ ID NO: 2552 |
| 104 | SEQ ID NO: 2582 | | SEQ ID NO: 2583 | | | SEQ ID NO: 2552 |
| 105 | SEQ ID NO: 2582 | | SEQ ID NO: 2583 | SEQ ID NO: 2550 | | SEQ ID NO: 2552 |
| 106 | SEQ ID NO: 2582 | | | SEQ ID NO: 2557 | | |
| 107 | SEQ ID NO: 2582 | | | SEQ ID NO: 2557 | SEQ ID NO: 2558 | |
| 108 | SEQ ID NO: 2582 | | | SEQ ID NO: 2557 | SEQ ID NO: 2558 | SEQ ID NO: 2559 |
| 109 | SEQ ID NO: 2582 | | | | SEQ ID NO: 2558 | |
| 110 | SEQ ID NO: 2582 | | | | SEQ ID NO: 2558 | SEQ ID NO: 2559 |
| 111 | SEQ ID NO: 2582 | | | | | SEQ ID NO: 2559 |
| 112 | SEQ ID NO: 2582 | | | SEQ ID NO: 2557 | | SEQ ID NO: 2559 |
| 113 | SEQ ID NO: 2582 | (I/V)KS | | SEQ ID NO: 2557 | | |
| 114 | SEQ ID NO: 2582 | (I/V)KS | | SEQ ID NO: 2557 | SEQ ID NO: 2558 | |
| 115 | SEQ ID NO: 2582 | (I/V)KS | | SEQ ID NO: 2557 | SEQ ID NO: 2558 | SEQ ID NO: 2559 |
| 116 | SEQ ID NO: 2582 | (I/V)KS | | | SEQ ID NO: 2558 | |
| 117 | SEQ ID NO: 2582 | (I/V)KS | | | SEQ ID NO: 2558 | SEQ ID NO: 2559 |
| 118 | SEQ ID NO: 2582 | (I/V)KS | | | | SEQ ID NO: 2559 |
| 119 | SEQ ID NO: 2582 | (I/V)KS | | SEQ ID NO: 2557 | | SEQ ID NO: 2559 |
| 120 | SEQ ID NO: 2582 | (I/V)KS | SEQ ID NO: 2583 | SEQ ID NO: 2557 | | |
| 121 | SEQ ID NO: 2582 | (I/V)KS | SEQ ID NO: 2583 | SEQ ID NO: 2557 | SEQ ID NO: 2558 | |
| 122 | SEQ ID NO: 2582 | (I/V)KS | SEQ ID NO: 2583 | SEQ ID NO: 2557 | SEQ ID NO: 2558 | SEQ ID NO: 2559 |
| 123 | SEQ ID NO: 2582 | (I/V)KS | SEQ ID NO: 2583 | | SEQ ID NO: 2558 | |
| 124 | SEQ ID NO: 2582 | (I/V)KS | SEQ ID NO: 2583 | | SEQ ID NO: 2558 | SEQ ID NO: 2559 |
| 125 | SEQ ID NO: 2582 | (I/V)KS | SEQ ID NO: 2583 | SEQ ID NO: 2557 | | SEQ ID NO: 2559 |
| 126 | SEQ ID NO: 2582 | (I/V)KS | SEQ ID NO: 2583 | | | SEQ ID NO: 2559 |
| 127 | | (I/V)KS | | SEQ ID NO: 2557 | | |
| 128 | | (I/V)KS | | SEQ ID NO: 2557 | SEQ ID NO: 2558 | |
| 129 | | (I/V)KS | | SEQ ID NO: 2557 | SEQ ID NO: 2558 | SEQ ID NO: 2559 |
| 130 | | (I/V)KS | | | SEQ ID NO: 2558 | |
| 131 | | (I/V)KS | | | SEQ ID NO: 2558 | SEQ ID NO: 2559 |
| 132 | | (I/V)KS | | | | SEQ ID NO: 2559 |
| 133 | | (I/V)KS | | SEQ ID NO: 2557 | | SEQ ID NO: 2559 |
| 134 | | (I/V)KS | SEQ ID NO: 2583 | SEQ ID NO: 2557 | | |
| 135 | | (I/V)KS | SEQ ID NO: 2583 | SEQ ID NO: 2557 | SEQ ID NO: 2558 | |
| 136 | | (I/V)KS | SEQ ID NO: 2583 | SEQ ID NO: 2557 | SEQ ID NO: 2558 | SEQ ID NO: 2559 |
| 137 | | (I/V)KS | SEQ ID NO: 2583 | | SEQ ID NO: 2558 | |
| 138 | | (I/V)KS | SEQ ID NO: 2583 | | SEQ ID NO: 2558 | SEQ ID NO: 2559 |
| 139 | | (I/V)KS | SEQ ID NO: 2583 | | | SEQ ID NO: 2559 |
| 140 | | (I/V)KS | SEQ ID NO: 2583 | SEQ ID NO: 2557 | | SEQ ID NO: 2559 |
| 141 | | | SEQ ID NO: 2583 | SEQ ID NO: 2557 | | |
| 142 | | | SEQ ID NO: 2583 | SEQ ID NO: 2557 | SEQ ID NO: 2558 | |
| 143 | | | SEQ ID NO: 2583 | SEQ ID NO: 2557 | SEQ ID NO: 2558 | SEQ ID NO: 2559 |
| 144 | | | SEQ ID NO: 2583 | | SEQ ID NO: 2558 | |
| 145 | | | SEQ ID NO: 2583 | | SEQ ID NO: 2558 | SEQ ID NO: 2559 |
| 146 | | | SEQ ID NO: 2583 | | | SEQ ID NO: 2559 |
| 147 | | | SEQ ID NO: 2583 | SEQ ID NO: 2557 | | SEQ ID NO: 2559 |
| 148 | SEQ ID NO: 2582 | | SEQ ID NO: 2583 | SEQ ID NO: 2557 | | |
| 149 | SEQ ID NO: 2582 | | SEQ ID NO: 2583 | SEQ ID NO: 2557 | SEQ ID NO: 2558 | |
| 150 | SEQ ID NO: 2582 | | SEQ ID NO: 2583 | SEQ ID NO: 2557 | SEQ ID NO: 2558 | SEQ ID NO: 2559 |
| 151 | SEQ ID NO: 2582 | | SEQ ID NO: 2583 | | SEQ ID NO: 2558 | |
| 152 | SEQ ID NO: 2582 | | SEQ ID NO: 2583 | | SEQ ID NO: 2558 | SEQ ID NO: 2559 |
| 153 | SEQ ID NO: 2582 | | SEQ ID NO: 2583 | | | SEQ ID NO: 2559 |
| 154 | SEQ ID NO: 2582 | | SEQ ID NO: 2583 | SEQ ID NO: 2557 | | SEQ ID NO: 2559 |
| 155 | SEQ ID NO: 2582 | | | SEQ ID NO: 2564 | | |
| 156 | SEQ ID NO: 2582 | | | SEQ ID NO: 2564 | SEQ ID NO: 2565 | |
| 157 | SEQ ID NO: 2582 | | | SEQ ID NO: 2564 | SEQ ID NO: 2565 | SEQ ID NO: 2566 |
| 158 | SEQ ID NO: 2582 | | | | SEQ ID NO: 2565 | |
| 159 | SEQ ID NO: 2582 | | | | SEQ ID NO: 2565 | SEQ ID NO: 2566 |
| 160 | SEQ ID NO: 2582 | | | | | SEQ ID NO: 2566 |
| 161 | SEQ ID NO: 2582 | | | SEQ ID NO: 2564 | | SEQ ID NO: 2566 |
| 162 | SEQ ID NO: 2582 | (I/V)KS | | SEQ ID NO: 2564 | | |
| 163 | SEQ ID NO: 2582 | (I/V)KS | | SEQ ID NO: 2564 | SEQ ID NO: 2565 | |
| 164 | SEQ ID NO: 2582 | (I/V)KS | | SEQ ID NO: 2564 | SEQ ID NO: 2565 | SEQ ID NO: 2566 |
| 165 | SEQ ID NO: 2582 | (I/V)KS | | | SEQ ID NO: 2565 | |
| 166 | SEQ ID NO: 2582 | (I/V)KS | | | SEQ ID NO: 2565 | SEQ ID NO: 2566 |
| 167 | SEQ ID NO: 2582 | (I/V)KS | | | | SEQ ID NO: 2566 |
| 168 | SEQ ID NO: 2582 | (I/V)KS | | SEQ ID NO: 2564 | | SEQ ID NO: 2566 |
| 169 | SEQ ID NO: 2582 | (I/V)KS | SEQ ID NO: 2583 | SEQ ID NO: 2564 | | |
| 170 | SEQ ID NO: 2582 | (I/V)KS | SEQ ID NO: 2583 | SEQ ID NO: 2564 | SEQ ID NO: 2565 | |
| 171 | SEQ ID NO: 2582 | (I/V)KS | SEQ ID NO: 2583 | SEQ ID NO: 2564 | SEQ ID NO: 2565 | SEQ ID NO: 2566 |
| 172 | SEQ ID NO: 2582 | (I/V)KS | SEQ ID NO: 2583 | | SEQ ID NO: 2565 | |
| 173 | SEQ ID NO: 2582 | (I/V)KS | SEQ ID NO: 2583 | | SEQ ID NO: 2565 | SEQ ID NO: 2566 |
| 174 | SEQ ID NO: 2582 | (I/V)KS | SEQ ID NO: 2583 | SEQ ID NO: 2564 | | SEQ ID NO: 2566 |
| 175 | SEQ ID NO: 2582 | (I/V)KS | SEQ ID NO: 2583 | | | SEQ ID NO: 2566 |
| 176 | | (I/V)KS | | SEQ ID NO: 2564 | | |
| 177 | | (I/V)KS | | SEQ ID NO: 2564 | SEQ ID NO: 2565 | |
| 178 | | (I/V)KS | | SEQ ID NO: 2564 | SEQ ID NO: 2565 | SEQ ID NO: 2566 |
| 179 | | (I/V)KS | | | SEQ ID NO: 2565 | |
| 180 | | (I/V)KS | | | SEQ ID NO: 2565 | SEQ ID NO: 2566 |
| 181 | | (I/V)KS | | | | SEQ ID NO: 2566 |
| 182 | | (I/V)KS | | SEQ ID NO: 2564 | | SEQ ID NO: 2566 |
| 183 | | (I/V)KS | SEQ ID NO: 2583 | SEQ ID NO: 2564 | | |
| 184 | | (I/V)KS | SEQ ID NO: 2583 | SEQ ID NO: 2564 | SEQ ID NO: 2565 | |
| 185 | | (I/V)KS | SEQ ID NO: 2583 | SEQ ID NO: 2564 | SEQ ID NO: 2565 | SEQ ID NO: 2566 |
| 186 | | (I/V)KS | SEQ ID NO: 2583 | | SEQ ID NO: 2565 | |
| 187 | | (I/V)KS | SEQ ID NO: 2583 | | SEQ ID NO: 2565 | SEQ ID NO: 2566 |
| 188 | | (I/V)KS | SEQ ID NO: 2583 | | | SEQ ID NO: 2566 |
| 189 | | (I/V)KS | SEQ ID NO: 2583 | SEQ ID NO: 2564 | | SEQ ID NO: 2566 |
| 190 | | | SEQ ID NO: 2583 | SEQ ID NO: 2564 | | |
| 191 | | | SEQ ID NO: 2583 | SEQ ID NO: 2564 | SEQ ID NO: 2565 | |
| 192 | | | SEQ ID NO: 2583 | SEQ ID NO: 2564 | SEQ ID NO: 2565 | SEQ ID NO: 2566 |
| 193 | | | SEQ ID NO: 2583 | | SEQ ID NO: 2565 | |
| 194 | | | SEQ ID NO: 2583 | | SEQ ID NO: 2565 | SEQ ID NO: 2566 |
| 195 | | | SEQ ID NO: 2583 | | | SEQ ID NO: 2566 |
| 196 | | | SEQ ID NO: 2583 | SEQ ID NO: 2564 | | SEQ ID NO: 2566 |
| 197 | SEQ ID NO: 2582 | | SEQ ID NO: 2583 | SEQ ID NO: 2564 | | |
| 198 | SEQ ID NO: 2582 | | SEQ ID NO: 2583 | SEQ ID NO: 2564 | SEQ ID NO: 2565 | |
| 199 | SEQ ID NO: 2582 | | SEQ ID NO: 2583 | SEQ ID NO: 2564 | SEQ ID NO: 2565 | SEQ ID NO: 2566 |
| 200 | SEQ ID NO: 2582 | | SEQ ID NO: 2583 | | SEQ ID NO: 2565 | |
| 201 | SEQ ID NO: 2582 | | SEQ ID NO: 2583 | | SEQ ID NO: 2565 | SEQ ID NO: 2566 |
| 202 | SEQ ID NO: 2582 | | SEQ ID NO: 2583 | | | SEQ ID NO: 2566 |
| 203 | SEQ ID NO: 2582 | | SEQ ID NO: 2583 | SEQ ID NO: 2564 | | SEQ ID NO: 2566 |
| 204 | SEQ ID NO: 2582 | | | SEQ ID NO: 2571 | | |
| 205 | SEQ ID NO: 2582 | | | SEQ ID NO: 2571 | SEQ ID NO: 2572 | |
| 206 | SEQ ID NO: 2582 | | | SEQ ID NO: 2571 | SEQ ID NO: 2572 | SEQ ID NO: 2573 |
| 207 | SEQ ID NO: 2582 | | | | SEQ ID NO: 2572 | |
| 208 | SEQ ID NO: 2582 | | | | SEQ ID NO: 2572 | SEQ ID NO: 2573 |
| 209 | SEQ ID NO: 2582 | | | | | SEQ ID NO: 2573 |
| 210 | SEQ ID NO: 2582 | | | SEQ ID NO: 2571 | | SEQ ID NO: 2573 |
| 211 | SEQ ID NO: 2582 | (I/V)KS | | SEQ ID NO: 2571 | | |
| 212 | SEQ ID NO: 2582 | (I/V)KS | | SEQ ID NO: 2571 | SEQ ID NO: 2572 | |
| 213 | SEQ ID NO: 2582 | (I/V)KS | | SEQ ID NO: 2571 | SEQ ID NO: 2572 | SEQ ID NO: 2573 |
| 214 | SEQ ID NO: 2582 | (I/V)KS | | | SEQ ID NO: 2572 | |
| 215 | SEQ ID NO: 2582 | (I/V)KS | | | SEQ ID NO: 2572 | SEQ ID NO: 2573 |
| 216 | SEQ ID NO: 2582 | (I/V)KS | | | | SEQ ID NO: 2573 |
| 217 | SEQ ID NO: 2582 | (I/V)KS | | SEQ ID NO: 2571 | | SEQ ID NO: 2573 |
| 218 | SEQ ID NO: 2582 | (I/V)KS | SEQ ID NO: 2583 | SEQ ID NO: 2571 | | |
| 219 | SEQ ID NO: 2582 | (I/V)KS | SEQ ID NO: 2583 | SEQ ID NO: 2571 | SEQ ID NO: 2572 | |
| 220 | SEQ ID NO: 2582 | (I/V)KS | SEQ ID NO: 2583 | SEQ ID NO: 2571 | SEQ ID NO: 2572 | SEQ ID NO: 2573 |
| 221 | SEQ ID NO: 2582 | (I/V)KS | SEQ ID NO: 2583 | | SEQ ID NO: 2572 | |
| 222 | SEQ ID NO: 2582 | (I/V)KS | SEQ ID NO: 2583 | | SEQ ID NO: 2572 | SEQ ID NO: 2573 |
| 223 | SEQ ID NO: 2582 | (I/V)KS | SEQ ID NO: 2583 | SEQ ID NO: 2571 | | SEQ ID NO: 2573 |
| 224 | SEQ ID NO: 2582 | (I/V)KS | SEQ ID NO: 2583 | | | SEQ ID NO: 2573 |
| 225 | | (I/V)KS | | SEQ ID NO: 2571 | | |
| 226 | | (I/V)KS | | SEQ ID NO: 2571 | SEQ ID NO: 2572 | |
| 227 | | (I/V)KS | | SEQ ID NO: 2571 | SEQ ID NO: 2572 | SEQ ID NO: 2573 |
| 228 | | (I/V)KS | | | SEQ ID NO: 2572 | |
| 229 | | (I/V)KS | | | SEQ ID NO: 2572 | SEQ ID NO: 2573 |
| 230 | | (I/V)KS | | | | SEQ ID NO: 2573 |
| 231 | | (I/V)KS | | SEQ ID NO: 2571 | | SEQ ID NO: 2573 |
| 232 | | (I/V)KS | SEQ ID NO: 2583 | SEQ ID NO: 2571 | | |
| 233 | | (I/V)KS | SEQ ID NO: 2583 | SEQ ID NO: 2571 | SEQ ID NO: 2572 | |
| 234 | | (I/V)KS | SEQ ID NO: 2583 | SEQ ID NO: 2571 | SEQ ID NO: 2572 | SEQ ID NO: 2573 |
| 235 | | (I/V)KS | SEQ ID NO: 2583 | | SEQ ID NO: 2572 | |
| 236 | | (I/V)KS | SEQ ID NO: 2583 | | SEQ ID NO: 2572 | SEQ ID NO: 2573 |
| 237 | | (I/V)KS | SEQ ID NO: 2583 | | | SEQ ID NO: 2573 |
| 238 | | (I/V)KS | SEQ ID NO: 2583 | SEQ ID NO: 2571 | | SEQ ID NO: 2573 |
| 239 | | | SEQ ID NO: 2583 | SEQ ID NO: 2571 | | |
| 240 | | | SEQ ID NO: 2583 | SEQ ID NO: 2571 | SEQ ID NO: 2572 | |
| 241 | | | SEQ ID NO: 2583 | SEQ ID NO: 2571 | SEQ ID NO: 2572 | SEQ ID NO: 2573 |
| 242 | | | SEQ ID NO: 2583 | | SEQ ID NO: 2572 | |
| 243 | | | SEQ ID NO: 2583 | | SEQ ID NO: 2572 | SEQ ID NO: 2573 |
| 244 | | | SEQ ID NO: 2583 | | | SEQ ID NO: 2573 |
| 245 | | | SEQ ID NO: 2583 | SEQ ID NO: 2571 | | SEQ ID NO: 2573 |
| 246 | SEQ ID NO: 2582 | | SEQ ID NO: 2583 | SEQ ID NO: 2571 | | |
| 247 | SEQ ID NO: 2582 | | SEQ ID NO: 2583 | SEQ ID NO: 2571 | SEQ ID NO: 2572 | |
| 248 | SEQ ID NO: 2582 | | SEQ ID NO: 2583 | SEQ ID NO: 2571 | SEQ ID NO: 2572 | SEQ ID NO: 2573 |
| 249 | SEQ ID NO: 2582 | | SEQ ID NO: 2583 | | SEQ ID NO: 2572 | |
| 250 | SEQ ID NO: 2582 | | SEQ ID NO: 2583 | | SEQ ID NO: 2572 | SEQ ID NO: 2573 |
| 251 | SEQ ID NO: 2582 | | SEQ ID NO: 2583 | | | SEQ ID NO: 2573 |
| 252 | SEQ ID NO: 2582 | | SEQ ID NO: 2583 | SEQ ID NO: 2571 | | SEQ ID NO: 2573 |

TABLE I4

| Comments | Sequence |
|---|---|
| HJ23.4 light chain variable | DVVMTQTPLSLPVSLGDQAFISCRSSQNLVHSNGNTYLHWYLQK |
| region | PGQSPKLLIYIVSNRFSGVPDRFSGSGSGTDFTLEISRVEAEDLGV |
| | YFCSQSTHVPLTFGAGTKLELK (SEQ ID NO: 2539) |
| HJ23.4 heavy chain variable | EVQLQQSGPDLVKPGASVKMSCKASGYTFTDYNIHWVKQSHGK |
| region | TLEWIGYINPNTGGTYYNQKFKGKATMTVNKSSSTAYMELRSLT |
| | SEDSAVYYCVATRWDGVNWAQGTLVTVSA (SEQ ID NO: 2540) |
| HJ23.4 L1 | QNLVHSNGNTY (SEQ ID NO: 2541) |
| HJ23.4 L2 | IVS |
| HJ23.4 L3 | SQSTHVPLT (SEQ ID NO: 2542) |
| HJ23.4 H1 | GYTFTDYN (SEQ ID NO: 2543) |
| HJ23.4 H2 | INPNTGGT (SEQ ID NO: 2544) |
| HJ23.4 H3 | VATRWDGVN (SEQ ID NO: 2545) |
| HJ23.7 light chain variable | DVVMTQTPLSLPVSLGDQASISCRSSQSLVHSNGNTYLHWYLQK |
| region | PGQSPKWYKVSNRFSGVPDRFSGSGSGTDFTLKISRVEAEDLGV |
| | YFCSQSTHVPLTFGAGTKLELK (SEQ ID NO: 2546) |
| HJ23.7 heavy chain variable | EVQLQQSGAELVKPGASVKLSCTSSGFNIKGYYIHWVKQRTEQG |
| region | LEWIGRIDPEDGETKNAPKFQGKATFGTDTFSNTAYLRLSSLTSE |
| | DTGVYYCVRTETRGAYWGPGTLVTVSA (SEQ ID NO: 2547) |
| HJ23.7 L1 | QSLVHSNGNTY (SEQ ID NO: 2548) |
| HJ23.7 L2 | KVS |
| HJ23.7 L3 | SQSTHVPLT (SEQ ID NO: 2549) |
| HJ23.7 H1 | GFNIKGYY (SEQ ID NO: 2550) |
| HJ23.7 H2 | IDPEDGET (SEQ ID NO: 2551) |
| HJ23.7 H3 | VRTETRGAY (SEQ ID NO: 2552) |
| HJ23.8 light chain variable | DVVMTQTPLSLPVSLGDQASISCRSSQSLVHSNGDTYLHWYLQK |
| region | RGQSPKWYKVSNRFSGVPDRFSGSGSGTDFTLKISRVEAEDLGV |
| | YFCSQTTHVPLTFGAGTKLELK (SEQ ID NO: 2553) |
| HJ23.8 heavy chain variable | QVPLQQPGAEFVKPGASVKLSCKASAYTFTRYWMHWVKQRPGR |
| region | GLEWIGRIDPNSGGTNYNEKFKSKATFTVDKPSSTSYMQLSSLTS |
| | EDSAVYFCVFTGTLFDYWGQGTTLTVSS (SEQ ID NO: 2554) |
| HJ23.8 L1 | QSLVHSNGDTY (SEQ ID NO: 2555) |
| HJ23.8 L2 | KVS |
| HJ23.8 L3 | SQTTHVPLT (SEQ ID NO: 2556) |
| HJ23.8 H1 | AYTFTRYW (SEQ ID NO: 2557) |
| HJ23.8 H2 | IDPNSGGT (SEQ ID NO: 2558) |
| HJ23.8 H3 | VFTGTLFDY (SEQ ID NO: 2559) |
| HJ23.9 light chain variable | DVVMTQTPLSLPVSLGDQASISCKSSQSLVHSNGNTYLHWYLQK |
| region | PGQSPKWYKVSNRFSGVPDRFSGSGSGTDFTLKISRVEAEDLGV |
| | YFCSQSTHVPPTFGGGTKLEIK (SEQ ID NO: 2560) |
| HJ23.9 heavy chain variable | EVQLQHSGPVLVKPGASVKMSCKSSGYTFTDYYLNWVKQSHGK |
| region | SPEWIGVINPNTGSTSYNQKFKGKATLTVDKSSSTAYMDLNSLTS |
| | EDSAVYYCATHYYGSIYKQAWFAYWGQGTLVT (SEQ ID NO: 2561) |
| HJ23.9 L1 | QSLVHSNGNTY (SEQ ID NO: 2562) |
| HJ23.9 L2 | KVS |
| HJ23.9 L3 | SQSTHVPPT (SEQ ID NO: 2563) |
| HJ23.9 H1 | GYTFTDYY (SEQ ID NO: 2564) |
| HJ23.9 H2 | INPNTGST (SEQ ID NO: 2565) |
| HJ23.9 H3 | ATHYYGSIYKQAWFAY (SEQ ID NO: 2566) |
| HJ23.10 light chain variable | DVVMTQTPLSLPVSLGDQASISCKSSQSLVHSNGNTYLHWYLQK |
| region | PGQSPKWYKVSNRFSGVPDRFSGSGSGTDFTLKISRVEAEDLGI |
| | YFCSQSTHVPPTFGGGTKLEIK (SEQ ID NO: 2567) |
| HJ23.10 heavy chain | EVQLQHSGPVLVKPGASVKMSCKASGYTFTDYYMNWVKQSHG |
| variable region | KSPEWIGVINPNTGSTSYNQKFKGKATLTVDKSSSTAYMDLNSLT |
| | SEDSAVYYCATHYYGSIYKQAWFAYWGQGTLVTV (SEQ ID NO: 2568) |
| HJ23.10 L1 | QSLVHSNGNTY (SEQ ID NO: 2569) |
| HJ23.10 L2 | KVS |
| HJ23.10 L3 | SQSTHVPPT (SEQ ID NO: 2570) |
| HJ23.10 H1 | GYTFTDYY (SEQ ID NO: 2571) |
| HJ23.10 H2 | INPNTGST (SEQ ID NO: 2572) |
| HJ23.10 H3 | ATHYYGSIYKQAWFAY (SEQ ID NO: 2573) |
| HJ23.13 light chain variable | DIVMSQSPSSLAVSVGEKVTMSCKSSQSLLYSSNLKNYLAWFQQ |
| region | KPGQSPKLLIYWASIRESGVPDRFTGSGSGTDFTLTINSVKAEDLA |
| | VYYCQQYYTFPLTFGAGTKLELK(SEQ ID NO: 2574) |
| HJ23.13 heavy chain | EVQLVETGGGLVQPKGSLKLSCAASGFSFNINAMHWVRQAPGT |
| variable region | GLKWVARIRSGSNDFATYYADSVKDRFTISRDDSHSMLYLQMN |
| | NLKTEDTAIYFCVREYVNYFVHWGQGTLVTVSA (SEQ ID NO: 2575) |
| HJ23.13 L1 | QSLLYSSNLKNY (SEQ ID NO: 2576) |
| HJ23.13 L2 | WAS |
| HJ23.13 L3 | QQYYTFPLT (SEQ ID NO: 2577) |
| HJ23.13 H1 | GFSFNINA (SEQ ID NO: 2578) |
| HJ23.13 H2 | IRSGSNDFAT (SEQ ID NO: 2579) |
| HJ23.13 H3 | VREYVNYFVH (SEQ ID NO: 2580) |
| | DHRDAGDLWFPGES (SEQ ID NO: 2581) |
| Consensus L1 | QX1LVHSNGX2TY, where X1 is S, T, N, or Q and X2 is D or N |
| | (SEQ ID NO: 2582) |
| Consensus L2 | X1VS, where X1 is I or K |
| Consensus L3 | SQX1THVPX2T, where X1 is S, T, N, or Q and X2 is P or L |
| | (SEQ ID NO: 2583) |

In some embodiments, each of the light chain variable regions and each of the heavy chain variable regions disclosed above, including those in Table 17B (e.g., antibody 1-378) and Table 17C (antibody 1-252) may be attached to the light chain constant regions (TABLE EN1) and heavy chain constant regions (TABLE EN2) to form complete antibody light and heavy chains, respectively, as further discussed below. Further, each of the generated heavy and light chain sequences may be combined to form a complete antibody structure. It should be understood that the heavy chain and light chain variable regions provided herein can also be attached to other constant domains having different sequences than the exemplary sequences listed herein.

J. PCT Patent Application Publication No. WO2020/079580A1

In some embodiments, the TREM2 agonist is an antibody, or an antigen-binding fragment thereof, as described in PCT Patent Application Publication No. WO2020/079580A1 (“the '580 application”), which is incorporated by reference herein, in its entirety.

In some embodiments, the TREM2 binding agent comprises an antibody that comprises a light chain variable domain comprising a CDRL1, CDRL2, and CDRL3, and a heavy chain variable domain comprising a CDRH1, CDRH2, and CDRH3 disclosed in the '580 application specification. In some embodiments, the TREM2 binding agent comprises an antibody that comprises a light chain variable domain and a heavy chain variable domain disclosed in the '580 application specification.

In some embodiments, the antibody or antigen-binding fragment thereof comprises:
a) a heavy chain variable region CDR1 comprising SEQ ID NO:2623 or SEQ ID NO:2626 or SEQ ID NO:2627 or SEQ ID NO:2629; a heavy chain variable region CDR2 comprising SEQ ID NO:2624 or SEQ ID NO:2628, or SEQ ID NO:2630; a heavy chain variable region CDR3 comprising SEQ ID NO:2625 or SEQ ID NO:2631; a light chain variable region CDR1 comprising SEQ ID NO:2636 or SEQ ID NO:2639 or SEQ ID NO:2642; a light chain variable region CDR2 comprising SEQ ID NO:2637 or SEQ ID NO:2640; and a light chain variable region CDR3 comprising SEQ ID NO:2638 or SEQ ID NO:2641;
b) a heavy chain variable region CDR1 comprising SEQ ID NO:2586 or SEQ ID NO:2589 or SEQ ID NO:2590 or SEQ ID NO:2592; a heavy chain variable region CDR2 comprising SEQ ID NO:2587 or SEQ ID NO:2591 or SEQ ID NO:2593; a heavy chain variable region CDR3 comprising SEQ ID NO:2588 or SEQ ID NO:2594; a light chain variable region CDR1 comprising SEQ ID NO:2599 or SEQ ID NO:2602 or SEQ ID NO:2605; a light chain variable region CDR2 comprising SEQ ID NO:2600 or SEQ ID NO:2603; and a light chain variable region CDR3 comprising SEQ ID NO:2601 or SEQ ID NO:2604;
c) a heavy chain variable region CDR1 comprising SEQ ID NO:2586 or SEQ ID NO:2589 or SEQ ID NO:2590 or SEQ ID NO:2592; a heavy chain variable region CDR2 comprising SEQ ID NO:2587 or SEQ ID NO:2591 or SEQ ID NO:2593; a heavy chain variable region CDR3 comprising SEQ ID NO:2588 or SEQ ID NO:2594; a light chain variable region CDR1 comprising SEQ ID NO:2599 or SEQ ID NO:2602 or SEQ ID NO:2605; a light chain variable region CDR2 comprising SEQ ID NO:2600 or SEQ ID NO:2603; and a light chain variable region CDR3 comprising SEQ ID NO:2660 or SEQ ID NO:2661; or
d) a heavy chain variable region CDR1 comprising SEQ ID NO:2666 or SEQ ID NO:2669 or SEQ ID NO:2670 or SEQ ID NO:2672; a heavy chain variable region CDR2 comprising SEQ ID NO:2667 or SEQ ID NO:2671 or SEQ ID NO:2673; a heavy chain variable region CDR3 comprising SEQ ID NO:2668 or SEQ ID NO:2674; a light chain variable region CDR1 comprising SEQ ID NO:2679 or SEQ ID NO:2682 or SEQ ID NO:2685; a light chain variable region CDR2 comprising SEQ ID NO:2680 or SEQ ID NO:2683; and a light chain variable region CDR3 comprising SEQ ID NO:2681 or SEQ ID NO:2684.

In some embodiments, the antibody or antigen-binding fragment thereof comprises:
a) a VH polypeptide sequence having at least 95% sequence identity to SEQ ID NO:2595 or to SEQ ID NO:2632, and a VL polypeptide sequence having at least 95% sequence identity to SEQ ID NO:2606 or to SEQ ID NO:2643; or
b) a VH polypeptide sequence having at least 95% sequence identity to SEQ ID NO:2595 or to SEQ ID NO:2675, and a VL polypeptide sequence having at least 95% sequence identity to SEQ ID NO:2662 or to SEQ ID NO:2686.

In some embodiments, the antibody or antigen-binding fragment thereof comprises:
a) a heavy chain variable region CDR1 comprising SEQ ID NO:2589; a heavy chain variable region CDR2 comprising SEQ ID NO:2587; a heavy chain variable region CDR3 comprising SEQ ID NO:2588; a light chain variable region CDR1 comprising SEQ ID NO:2599; a light chain variable region CDR2 comprising SEQ ID NO:2600; and a light chain variable region CDR3 comprising SEQ ID NO:2601; b) a heavy chain variable region CDR1 comprising SEQ ID NO:2626; a heavy chain variable region CDR2 comprising SEQ ID NO:2624; a heavy chain variable region CDR3 comprising SEQ ID NO:2625; a light chain variable region CDR1 comprising SEQ ID NO:2636; a light chain variable region CDR2 comprising, e.g., consisting of SEQ ID NO:2637; and a light chain variable region CDR3 comprising SEQ ID NO:2638; c) a heavy chain variable region CDR1 comprising SEQ ID NO:2589; a heavy chain variable region CDR2 comprising SEQ ID NO:2587; a heavy chain variable region CDR3 comprising SEQ ID NO:2588; a light chain variable region CDR1 comprising SEQ ID NO:2599; a light chain variable region CDR2 comprising SEQ ID NO:2600; and a light chain variable region CDR3 comprising SEQ ID NO:2660; or d) a heavy chain variable region CDR1 comprising SEQ ID NO:2669; a heavy chain variable region CDR2 comprising SEQ ID NO:2667; a heavy chain variable region CDR3 comprising SEQ ID NO:2668; a light chain variable region CDR1 comprising SEQ ID NO:2679; a light chain variable region CDR2 comprising SEQ ID NO:2680; and a light chain variable region CDR3 comprising SEQ ID NO:2681.

In some embodiments, the antibody or antigen-binding fragment thereof comprises:
a) a heavy chain variable region CDR1 of SEQ ID NO:2590; a heavy chain variable region CDR2 comprising SEQ ID NO:2591; a heavy chain variable region CDR3 comprising SEQ ID NO:2588; a light chain variable region CDR1 comprising SEQ ID NO:2602; a light chain variable region CDR2 comprising SEQ ID NO:2603; and a light chain variable region CDR3 comprising SEQ ID NO:2604;
b) a heavy chain variable region CDR1 comprising SEQ ID NO:2627; a heavy chain variable region CDR2 comprising SEQ ID NO:2628; a heavy chain variable region CDR3 comprising SEQ ID NO:2625; a light chain variable region CDR1 comprising SEQ ID NO:2639; a light chain variable region CDR2 comprising SEQ ID NO:2640; and a light chain variable region CDR3 comprising SEQ ID NO:2641;
c) a heavy chain variable region CDR1 comprising SEQ ID NO:2590; a heavy chain variable region CDR2 comprising SEQ ID NO:2591; a heavy chain variable region CDR3 comprising SEQ ID NO:2588; a light chain variable region CDR1 comprising SEQ ID NO:2602; a light chain variable region CDR2 comprising SEQ ID NO:2603; and a light chain variable region CDR3 comprising SEQ ID NO:2661; or
d) a heavy chain variable region CDR1 comprising SEQ ID NO:2670; a heavy chain variable region CDR2 comprising SEQ ID NO:2671; a heavy chain variable region CDR3 comprising SEQ ID NO:2668; a light chain variable region CDR1 comprising SEQ ID NO:2682; a light chain variable region CDR2 comprising SEQ ID NO:2683; and a light chain variable region CDR3 comprising SEQ ID NO:2684.

In some embodiments, the antibody or antigen-binding fragment thereof comprises:
a) a heavy chain variable region CDR1 comprising SEQ ID NO:2592; a heavy chain variable region CDR2 comprising SEQ ID NO:2593; a heavy chain variable region CDR3 comprising SEQ ID NO:2594; a light chain variable region CDR1 comprising SEQ ID NO:2605; a light chain variable region CDR2 comprising SEQ ID NO:2603; and a light chain variable region CDR3 comprising SEQ ID NO:2601;
b) a heavy chain variable region CDR1 comprising SEQ ID NO:2629; a heavy chain variable region CDR2 comprising SEQ ID NO:2630; a heavy chain variable region CDR3 comprising SEQ ID NO:2631; a light chain variable region CDR1 comprising SEQ ID NO:2642; a light chain variable region CDR2 comprising SEQ ID NO:2640; and a light chain variable region CDR3 comprising SEQ ID NO:2638;
c) a heavy chain variable region CDR1 comprising SEQ ID NO:2592; a heavy chain variable region CDR2 comprising SEQ ID NO:2593; a heavy chain variable region CDR3 comprising SEQ ID NO:2594; a light chain variable region CDR1 comprising SEQ ID NO:2605; a light chain variable region CDR2 comprising SEQ ID NO:2603; and a light chain variable region CDR3 comprising SEQ ID NO:2660; or
d) a heavy chain variable region CDR1 comprising SEQ ID NO:2672; a heavy chain variable region CDR2 comprising SEQ ID NO:2673; a heavy chain variable region CDR3 comprising SEQ ID NO:2674; a light chain variable region CDR1 comprising SEQ ID NO:2685; a light chain variable region CDR2 comprising SEQ ID NO:2683; and a light chain variable region CDR3 comprising SEQ ID NO:2681.

In some embodiments, the antibody or antigen-binding fragment thereof comprises:
a) a heavy chain variable region CDR1 comprising SEQ ID NO:2586; a heavy chain variable region CDR2 comprising SEQ ID NO:2587; a heavy chain variable region CDR3 comprising SEQ ID NO:2588; a light chain variable region CDR1 comprising SEQ ID NO:2599; a light chain variable region CDR2 comprising SEQ ID NO:2600; and a light chain variable region CDR3 comprising SEQ ID NO:2601;
b) a heavy chain variable region CDR1 comprising SEQ ID NO:2623; a heavy chain variable region CDR2 comprising SEQ ID NO:2624; a heavy chain variable region CDR3 comprising SEQ ID NO:2625; a light chain variable region CDR1 comprising SEQ ID NO:2636; a light chain variable region CDR2 comprising SEQ ID NO:2637; and a light chain variable region CDR3 comprising SEQ ID NO:2638;
c) a heavy chain variable region CDR1 comprising SEQ ID NO:2586; a heavy chain variable region CDR2 comprising SEQ ID NO:2587; a heavy chain variable region CDR3 comprising SEQ ID NO:2588; a light chain variable region CDR1 comprising SEQ ID NO:2599; a light chain variable region CDR2 comprising SEQ ID NO:2600; and a light chain variable region CDR3 comprising SEQ ID NO:2660; or
d) a heavy chain variable region CDR1 comprising SEQ ID NO:2666; a heavy chain variable region CDR2 comprising SEQ ID NO:2667; a heavy chain variable region CDR3 comprising SEQ ID NO:2668; a light chain variable region CDR1 comprising SEQ ID NO:2679; a light chain variable region CDR2 comprising SEQ ID NO:2680; and a light chain variable region CDR3 comprising SEQ ID NO:2681.

In some embodiments, the antibody or antigen-binding fragment thereof comprises:
a) a VH comprising SEQ ID NO:2595 and a VL comprising SEQ ID NO:2606; or
b) a VH comprising SEQ ID NO:2632 and a VL comprising SEQ ID NO:2643; or
c) a VH comprising a sequence having at least 95% homology to SEQ ID NO:2595 and a VL comprising a sequence having at least 95% homology to SEQ ID NO:2606; or
d) a VH comprising a sequence having at least 95% homology to SEQ ID NO:2632 and a VL comprising a sequence having at least 95% homology to SEQ ID NO:2643; or
e) a VH comprising, e.g. consisting of, a sequence that differs by at least 1, 2, 3, 4, 5, or 6 amino acids from SEQ ID NO:2595 and a VL comprising, e.g. consisting of, a sequence that differs by at least 1, 2, 3, 4, 5, or 6 amino acids from SEQ ID NO:2606; or
f) a VH comprising, e.g. consisting of, a sequence that differs by at least 1, 2, 3, 4, 5, or 6 amino acids from SEQ ID NO:2632 and a VL comprising, e.g. consisting of, a sequence that differs by at least 1, 2, 3, 4, 5, or 6 amino acids from SEQ ID NO:2643. g) a VH comprising SEQ ID NO:2595 and a VL comprising SEQ ID NO:2662; or
h) a VH comprising SEQ ID NO:2675 and a VL comprising SEQ ID NO:2686; or
i) a VH comprising a sequence having at least 95% homology to SEQ ID NO:2595 and a VL comprising a sequence having at least 95% homology to SEQ ID NO:2662; or
j) a VH comprising a sequence having at least 95% homology to SEQ ID NO:2675 and a VL comprising a sequence having at least 95% homology to SEQ ID NO:2686; or
k) a VH comprising, e.g. consisting of, a sequence that differs by at least 1, 2, 3, 4, 5, or 6 amino acids from SEQ ID NO:2595 and a VL comprising, e.g. consisting of, a sequence that differs by at least 1, 2, 3, 4, 5, or 6 amino acids from SEQ ID NO:2662; or
l) a VH comprising, e.g. consisting of, a sequence that differs by at least 1, 2, 3, 4, 5, or 6 amino acids from SEQ ID NO:2675 and a VL comprising, e.g. consisting of, a sequence that differs by at least 1, 2, 3, 4, 5, or 6 amino acids from SEQ ID NO:2686.

In some embodiments, the antibody or antigen-binding fragment thereof comprises:
a) a heavy chain amino acid sequence comprising SEQ ID NO:2597, SEQ ID NO:2611, SEQ ID NO:2615, SEQ ID NO:2617, SEQ ID NO:2619, or SEQ ID NO:2621, and a light chain amino acid sequence comprising SEQ ID NO:2608; b) a heavy chain amino acid sequence comprising SEQ ID NO:2634, SEQ ID NO:2648, SEQ ID NO:2652, SEQ ID NO:2654, SEQ ID NO:2656, or SEQ ID NO:2658, and a light chain amino acid sequence comprising SEQ ID NO:2645; c) a heavy chain amino acid sequence having at least 95% sequence identity to SEQ ID NO:2597, SEQ ID NO:2611, SEQ ID NO:2615, SEQ ID NO:2617, SEQ ID NO:2619, or SEQ ID NO:2621, and a light chain amino acid sequence having at least 95% sequence identity to SEQ ID NO:2608;
d) a heavy chain amino acid sequence having at least 95% sequence identity to SEQ ID NO:2634, SEQ ID NO:2648, SEQ ID NO:2652, SEQ ID NO:2654, SEQ ID NO:2656, or SEQ ID NO:2658, and a light chain amino acid sequence having at least 95% sequence identity to SEQ ID NO:2645;
e) a heavy chain amino acid sequence comprising SEQ ID NO:2597, and a light chain amino acid sequence comprising SEQ ID NO:2664;
f) a heavy chain amino acid sequence comprising SEQ ID NO:2677, and a light chain amino acid sequence comprising SEQ ID NO:2688;
g) a heavy chain amino acid sequence having at least 95% sequence identity to SEQ ID NO:2597, and a light chain amino acid sequence having at least 95% sequence identity to SEQ ID NO:2664; or
h) a heavy chain amino acid sequence having at least 95% sequence identity to SEQ ID NO:2677, and a light chain amino acid sequence having at least 95% sequence identity to SEQ ID NO:2688.

In some embodiments, the antibody or antigen-binding fragment thereof comprises:
a) a heavy chain sequence comprising SEQ ID NO:2597 and a light chain sequence comprising SEQ ID NO:2608;
b) a heavy chain sequence comprising SEQ ID NO:2611 and a light chain sequence comprising SEQ ID NO:2608;
c) a heavy chain sequence comprising SEQ ID NO:2615 and a light chain sequence comprising SEQ ID NO:2608;
d) a heavy chain sequence comprising SEQ ID NO:2617 and a light chain sequence comprising SEQ ID NO:2608;
e) a heavy chain sequence comprising SEQ ID NO:2619 and a light chain sequence comprising SEQ ID NO:2608;
f) a heavy chain sequence comprising SEQ ID NO:2621 and a light chain sequence comprising SEQ ID NO:2608;
g) a heavy chain sequence comprising SEQ ID NO:2634 and a light chain sequence comprising SEQ ID NO:2645;
h) a heavy chain sequence comprising SEQ ID NO:2648 and light chain sequence comprising SEQ ID NO:2645;
i) a heavy chain sequence comprising SEQ ID NO:2652 and light chain sequence comprising SEQ ID NO:2645;
j) a heavy chain sequence comprising SEQ ID NO:2654 and light chain sequence comprising SEQ ID NO:2645;
k) a heavy chain sequence comprising SEQ ID NO:2656 and light chain sequence comprising SEQ ID NO:2645;
l) a heavy chain sequence comprising SEQ ID NO:2658 and light chain sequence comprising SEQ ID NO:2645;
m) a heavy chain sequence comprising SEQ ID NO:2597 and light chain sequence comprising SEQ ID NO:2664; or
n) a heavy chain sequence comprising SEQ ID NO:2677 and light chain sequence comprising SEQ ID NO:2688.

In some embodiments, the antibody is an antibody disclosed in Table 1 of PCT Patent Application Publication No. WO2020/079580A1, reproduced below as TABLE J1.

TABLE J1
Sequences of Exemplary Monoclonal Antibodies That Bind Human TREM2

| | |
|---|---|
| Description | Sequence |

MOR44698A

| | |
|---|---|
| HCDR1 | GYTFTGYHMS (SEQ ID NO: 2586) |
| (Combined) | |
| HCDR2 | VINPVSGNTVYAQKFQG (SEQ ID NO: 2587) |
| (Combined) | |
| HCDR3 | IPSYTYAFDY (SEQ ID NO: 2588) |
| (Combined) | |
| HCDR1 | GYHMS (SEQ ID NO: 2589) |
| (Kabat) | |
| HCDR2 | VINPVSGNTVYAQKFQG (SEQ ID NO: 2587) |
| (Kabat) | |
| HCDR3 | IPSYTYAFDY (SEQ ID NO: 2588) |
| (Kabat) | |
| HCDR1 | GYTFTGY (SEQ ID NO: 2590) |
| (Chothia) | |
| HCDR2 | NPVSGN (SEQ ID NO: 2591) |
| (Chothia) | |
| HCDR3 | IPSYTYAFDY (SEQ ID NO: 2588) |
| (Chothia) | |
| HCDR1 | GYTFTGYH (SEQ ID NO: 2592) |
| (IMGT) | |
| HCDR2 | INPVSGNT (SEQ ID NO: 2593) |
| (IMGT) | |
| HCDR3 | ARIPSYTYAFDY (SEQ ID NO: 2594) |
| (IMGT) | |
| VH | QVQLVQSGAEVKKPGASVKVSCKASGYTFTGYHMSWVRQAPGQGLEWMGV |
| | INPVSGNTVYAQKFQGRVTMTRDTSISTAYMELSRLRSEDTAVYYCARIPSYT |
| | YAFDYWGQGTLVTVSS (SEQ ID NO: 2595) |
| DNA VH | CAGGTGCAATTGGTGCAGAGCGGTGCGGAAGTGAAAAAACCGGGTGCCAG |
| | CGTGAAAGTTAGCTGCAAAGCGTCCGGATATACCTTCACTGGTTACCATAT |
| | GTCTTGGGTGCGCCAGGCCCCGGGCCAGGGCCTCGAGTGGATGGGCGTTAT |
| | CAACCCGGTTTCTGGCAACACGGTTTACGCGCAGAAATTTCAGGGCCGGGT |
| | GACCATGACCCGTGATACCAGCATTAGCACCGCGTATATGGAACTGAGCC |
| | GTCTGCGTAGCGAAGATACGGCCGTGTATTATTGCGCGCGTATCCCGTCTT |
| | ACACTTACGCTTTCGATTACTGGGGCCAAGGCACCCTGGTGACTGTTAGCT |
| | CA (SEQ ID NO: 2596) |
| Heavy Chain | QVQLVQSGAEVKKPGASVKVSCKASGYTFTGYHMSWVRQAPGQGLEWMGV |
| | INPVSGNTVYAQKFQGRVTMTRDTSISTAYMELSRLRSEDTAVYYCARIPSYT |
| | YAFDYWGQGTLVTVSSASTKGPSVFPLAPSSKSTSGGTAALGCLVKDYFPEPV |
| | TVSWNSGALTSGVHTFPAVLQSSGLYSLSSVVTVPSSSLGTQTYICNVNHKPSN |
| | TKVDKRVEPKSCDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSREEMTKNQVSL |
| | TCLVKGFYPSDIAVEWESNGQPENNYKTTPPVLDSDGSFFLYSKLTVDKSRWQ |
| | QGNVFSCSVMHEALHNHYTQKSLSLSPGK (SEQ ID NO: 2597) |
| DNA Heavy | CAGGTGCAATTGGTGCAGAGCGGTGCGGAAGTGAAAAAACCGGGTGCCAG |
| Chain | CGTGAAAGTTAGCTGCAAAGCGTCCGGATATACCTTCACTGGTTACCATAT |
| | GTCTTGGGTGCGCCAGGCCCCGGGCCAGGGCCTCGAGTGGATGGGCGTTAT |
| | CAACCCGGTTTCTGGCAACACGGTTTACGCGCAGAAATTTCAGGGCCGGGT |
| | GACCATGACCCGTGATACCAGCATTAGCACCGCGTATATGGAACTGAGCC |
| | GTCTGCGTAGCGAAGATACGGCCGTGTATTATTGCGCGCGTATCCCGTCTT |
| | ACACTTACGCTTTCGATTACTGGGGCCAAGGCACCCTGGTGACTGTTAGCT |
| | CAGCCTCCACCAAGGGTCCATCGGTCTTCCCCCTGGCACCCTCCTCCAAGA |
| | GCACCTCTGGGGGCACAGCGGCCCTGGGCTGCCTGGTCAAGGACTACTTCC |
| | CCGAACCGGTGACGGTGTCGTGGAACTCAGGCGCCCTGACCAGCGGCGTG |
| | CACACCTTCCCGGCTGTCCTACAGTCCTCAGGACTCTACTCCCTCAGCAGC |
| | GTGGTGACCGTGCCCTCCAGCAGCTTGGGCACCCAGACCTACATCTGCAAC |
| | GTGAATCACAAGCCCAGCAACACCAAGGTGGACAAGAGAGTTGAGCCCAA |
| | ATCTTGTGACAAAACTCACACATGCCCACCGTGCCCAGCACCTGAAGCAGC |
| | GGGGGGACCGTCAGTCTTCCTCTTCCCCCCAAAACCCAAGGACACCCTCAT |
| | GATCTCCCGGACCCCTGAGGTCACATGCGTGGTGGTGGACGTGAGCCACG |
| | AAGACCCTGAGGTCAAGTTCAACTGGTACGTGGACGGCGTGGAGGTGCAT |
| | AATGCCAAGACAAAGCCGCGGGAGGAGCAGTACAACAGCACGTACCGGGT |
| | GGTCAGCGTCCTCACCGTCCTGCACCAGGACTGGCTGAATGGCAAGGAGT |
| | ACAAGTGCAAGGTCTCCAACAAAGCCCTCCCAGCCCCCATCGAGAAAACC |
| | ATCTCCAAAGCCAAAGGGCAGCCCCGAGAACCACAGGTGTACACCCTGCC |
| | CCCATCCCGGGAGGAGATGACCAAGAACCAGGTCAGCCTGACCTGCCTGG |
| | TCAAAGGCTTCTATCCCAGCGACATCGCCGTGGAGTGGGAGAGCAATGGG |
| | CAGCCGGAGAACAACTACAAGACCACGCCTCCCGTGCTGGACTCCGACGG |
| | CTCCTTCTTCCTCTACAGCAAGCTCACCGTGGACAAGAGCAGGTGGCAGCA |
| | GGGGAACGTCTTCTCATGCTCCGTGATGCATGAGGCTCTGCACAACCACTA |
| | CACGCAGAAGAGCCTCTCCCTGTCTCCGGGTAAA (SEQ ID NO: 2598) |
| LCDR1 | RASQDISNYLA (SEQ ID NO: 2599) |
| (Combined) | |
| LCDR2 | RASSLQS (SEQ ID NO: 2600) |
| (Combined) | |
| LCDR3 | FQYRHMPSQT (SEQ ID NO: 2601) |
| (Combined) | |
| LCDR1 | RASQDISNYLA (SEQ ID NO: 2599) |
| (Kabat) | |
| LCDR2 | RASSLQS (SEQ ID NO: 2600) |
| (Kabat) | |
| LCDR3 | FQYRHMPSQT (SEQ ID NO: 2601) |
| (Kabat) | |
| LCDR1 | SQDISNY (SEQ ID NO: 2602) |
| (Chothia) | |
| LCDR2 | RAS (SEQ ID NO: 2603) |
| (Chothia) | |
| LCDR3 | YRHMPSQ (SEQ ID NO: 2604) |
| (Chothia) | |
| LCDR1 | QDISNY (SEQ ID NO: 2605) |
| (IMGT) | |
| LCDR2 | RAS (SEQ ID NO: 2603) |
| (IMGT) | |
| LCDR3 | FQYRHMPSQT (SEQ ID NO: 2601) |
| (IMGT) | |
| VL | DIQMTQSPSSLSASVGDRVTITCRASQDISNYLAWYQQKPGKAPKLLIYRASSL |
| | QSGVPSRFSGSGSGTDFTLTISSLQPEDFATYYCFQYRHMPSQTFGQGTKVEIK |
| | (SEQ ID NO: 2606) |
| DNA VL | GATATCCAGATGACCCAGAGCCCGAGCAGCCTGAGCGCCAGCGTGGGCGA |
| | TCGCGTGACCATTACCTGCAGAGCCAGCCAGGACATTTCTAACTACCTGGC |
| | TTGGTACCAGCAGAAACCGGGCAAAGCGCCGAAACTATTAATCTACCGTG |
| | CTTCTTCTCTGCAAAGCGGCGTGCCGAGCCGCTTTAGCGGCAGCGGATCCG |
| | GCACCGATTTCACCCTGACCATTAGCTCTCTGCAACCGGAAGACTTTGCGA |
| | CCTATTATTGCTTCCAGTACCGTCATATGCCGTCTCAGACCTTTGGCCAGGG |
| | CACGAAAGTTGAAATTAAA (SEQ ID NO: 2607) |
| Light Chain | DIQMTQSPSSLSASVGDRVTITCRASQDISNYLAWYQQKPGKAPKLLIYRASSL |
| | QSGVPSRFSGSGSGTDFTLTISSLQPEDFATYYCFQYRHMPSQTFGQGTKVEIK |
| | RTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGE |
| | C (SEQ ID NO: 2608) |
| DNA Light | GATATCCAGATGACCCAGAGCCCGAGCAGCCTGAGCGCCAGCGTGGGCGA |
| Chain | TCGCGTGACCATTACCTGCAGAGCCAGCCAGGACATTTCTAACTACCTGGC |
| | TTGGTACCAGCAGAAACCGGGCAAAGCGCCGAAACTATTAATCTACCGTG |
| | CTTCTTCTCTGCAAAGCGGCGTGCCGAGCCGCTTTAGCGGCAGCGGATCCG |
| | GCACCGATTTCACCCTGACCATTAGCTCTCTGCAACCGGAAGACTTTGCGA |
| | CCTATTATTGCTTCCAGTACCGTCATATGCCGTCTCAGACCTTTGGCCAGGG |
| | CACGAAAGTTGAAATTAAACGTACGGTGGCCGCTCCCAGCGTGTTCATCTT |
| | CCCCCCCAGCGACGAGCAGCTGAAGAGCGGCACCGCCAGCGTGGTGTGCC |
| | TGCTGAACAACTTCTACCCCCGGGAGGCCAAGGTGCAGTGGAAGGTGGAC |
| | AACGCCCTGCAGAGCGGCAACAGCCAGGAAAGCGTCACCGAGCAGGACA |
| | GCAAGGACTCCACCTACAGCCTGAGCAGCACCCTGACCCTGAGCAAGGCC |
| | GACTACGAGAAGCACAAGGTGTACGCCTGCGAGGTGACCCACCAGGGCCT |
| | GTCCAGCCCCGTGACCAAGAGCTTCAACCGGGGCGAGTGT (SEQ ID NO: 2609) |

MOR44698B

| | |
|---|---|
| HCDR1 | GYTFTGYHMS (SEQ ID NO: 2586) |
| (Combined) | |
| HCDR2 | VINPVSGNTVYAQKFQG (SEQ ID NO: 2587) |
| (Combined) | |
| HCDR3 | IPSYTYAFDY (SEQ ID NO: 2588) |
| (Combined) | |
| HCDR1 | GYHMS (SEQ ID NO: 2589) |
| (Kabat) | |
| HCDR2 | VINPVSGNTVYAQKFQG (SEQ ID NO: 2587) |
| (Kabat) | |
| HCDR3 | IPSYTYAFDY (SEQ ID NO: 2588) |
| (Kabat) | |
| HCDR1 | GYTFTGY (SEQ ID NO: 2590) |
| (Chothia) | |
| HCDR2 | NPVSGN (SEQ ID NO: 2591) |
| (Chothia) | |
| HCDR3 | IPSYTYAFDY (SEQ ID NO: 2588) |
| (Chothia) | |
| HCDR1 | GYTFTGYH (SEQ ID NO: 2592) |
| (IMGT) | |
| HCDR2 | INPVSGNT (SEQ ID NO: 2593) |
| (IMGT) | |
| HCDR3 | ARIPSYTYAFDY (SEQ ID NO: 2594) |
| (IMGT) | |
| VH | QVQLVQSGAEVKKPGASVKVSCKASGYTFTGYHMSWVRQAPGQGLEWMGV |
| | INPVSGNTVYAQKFQGRVTMTRDTSISTAYMELSRLRSEDTAVYYCARIPSYT |
| | YAFDYWGQGTLVTVSS (SEQ ID NO: 2595) |
| DNA VH | CAAGTGCAACTCGTGCAGTCAGGAGCCGAAGTCAAGAAGCCTGGAGCCTC |
| | GGTCAAGGTGTCCTGCAAGGCCAGCGGATACACTTTCACTGGATACCACAT |
| | GTCGTGGGTCAGACAGGCTCCTGGCCAAGGGCTGGAGTGGATGGGCGTCA |
| | TCAACCCGGTGTCGGGTAATACCGTGTACGCCCAGAAGTTCCAGGGTCGCG |
| | TGACCATGACCCGGGATACCTCCATTAGCACCGCGTACATGGAGCTCAGCC |
| | GGTTGAGATCCGAGGATACCGCCGTGTACTACTGTGCGCGGATCCCGTCCT |
| | ACACTTACGCCTTCGACTATTGGGGCCAGGGGACTCTTGTCACCGTGTCCT |
| | CG (SEQ ID NO: 2610) |
| Heavy Chain | QVQLVQSGAEVKKPGASVKVSCKASGYTFTGYHMSWVRQAPGQGLEWMGV |
| | INPVSGNTVYAQKFQGRVTMTRDTSISTAYMELSRLRSEDTAVYYCARIPSYT |
| | YAFDYWGQGTLVTVSSASTKGPSVFPLAPSSKSTSGGTAALGCLVKDYFPEPV |
| | TVSWNSGALTSGVHTFPAVLQSSGLYSLSSVVTVPSSSLGTQTYICNVNHKPSN |
| | TKVDKRVEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSREEMTKNVSL |
| | TCLVKGFYPSDIAVEWESNGQPENNYKTTPPVLDSDGSFFLYSKLTVDKSRWQ |
| | QGNVFSCSVMHEALHNHYTQKSLSLSPGK (SEQ ID NO: 2611) |
| DNA Heavy | CAAGTGCAACTCGTGCAGTCAGGAGCCGAAGTCAAGAAGCCTGGAGCCTC |
| Chain | GGTCAAGGTGTCCTGCAAGGCCAGCGGATACACTTTCACTGGATACCACAT |
| | GTCGTGGGTCAGACAGGCTCCTGGCCAAGGGCTGGAGTGGATGGGCGTCA |
| | TCAACCCGGTGTCGGGTAATACCGTGTACGCCCAGAAGTTCCAGGGTCGCG |
| | TGACCATGACCCGGGATACCTCCATTAGCACCGCGTACATGGAGCTCAGCC |
| | GGTTGAGATCCGAGGATACCGCCGTGTACTACTGTGCGCGGATCCCGTCCT |
| | ACACTTACGCCTTCGACTATTGGGGCCAGGGGACTCTTGTCACCGTGTCCT |
| | CGGCCTCCACTAAGGGCCCAAGTGTGTTTCCCCTGGCCCCCAGCAGCAAGT |
| | CTACTTCCGGCGGAACTGCTGCCCTGGGTTGCCTGGTGAAGGACTACTTCC |
| | CCGAGCCCGTGACAGTGTCCTGGAACTCTGGGGCTCTGACTTCCGGCGTGC |
| | ACACCTTCCCCGCCGTGCTGCAGAGCAGCGGCCTGTACAGCCTGAGCAGCG |
| | TGGTGACAGTGCCCTCCAGCTCTCTGGGAACCCAGACCTATATCTGCAACG |
| | TGAACCACAAGCCCAGCAACACCAAGGTGGACAAGAGAGTGGAGCCCAA |
| | GAGCTGCGACAAGACCCACACCTGCCCCCCCTGCCCAGCTCCAGAACTGCT |
| | GGGAGGGCCTTCCGTGTTCCTGTTCCCCCCCAAGCCCAAGGACACCCTGAT |
| | GATCAGCAGGACCCCCGAGGTGACCTGCGTGGTGGTGGACGTGTCCCACG |
| | AGGACCCAGAGGTGAAGTTCAACTGGTACGTGGACGGCGTGGAGGTGCAC |
| | AACGCCAAGACCAAGCCCAGAGAGGAGCAGTACAACAGCACCTACAGGGT |
| | GGTGTCCGTGCTGACCGTGCTGCACCAGGACTGGCTGAACGGCAAAGAAT |
| | ACAAGTGCAAAGTCTCCAACAAGGCCCTGCCAGCCCCAATCGAAAAGACA |
| | ATCAGCAAGGCCAAGGGCCAGCCACGGGAGCCCCAGGTGTACACCCTGCC |
| | CCCCAGCCGGGAGGAGATGACCAAGAACCAGGTGTCCCTGACCTGTCTGG |
| | TGAAGGGCTTCTACCCCAGCGATATCGCCGTGGAGTGGGAGAGCAACGGC |
| | CAGCCCGAGAACAACTACAAGACCACCCCCCCAGTGCTGGACAGCGACGG |
| | CAGCTTCTTCCTGTACAGCAAGCTGACCGTGGACAAGTCCAGGTGGCAGCA |
| | GGGCAACGTGTTCAGCTGCAGCGTGATGCACGAGGCCCTGCACAACCACT |
| | ACACCCAGAAGTCCCTGAGCCTGAGCCCCGGCAAG (SEQ ID NO: 2612) |
| LCDR1 | RASQDISNYLA (SEQ ID NO: 2599) |
| (Combined) | |
| LCDR2 | RASSLQS (SEQ ID NO: 2600) |
| (Combined) | |
| LCDR3 | FQYRHMPSQT (SEQ ID NO: 2601) |
| (Combined) | |
| LCDR1 | RASQDISNYLA (SEQ ID NO: 2599) |
| (Kabat) | |
| LCDR2 | RASSLQS (SEQ ID NO: 2600) |
| (Kabat) | |
| LCDR3 | FQYRHMPSQT (SEQ ID NO: 2601) |
| (Kabat) | |
| LCDR1 | SQDISNY (SEQ ID NO: 2602) |
| (Chothia) | |
| LCDR2 | RAS (SEQ ID NO: 2603) |
| (Chothia) | |
| LCDR3 | YRHMPSQ (SEQ ID NO: 2604) |
| (Chothia) | |
| LCDR1 | QDISNY (SEQ ID NO: 2605) |
| (IMGT) | |
| LCDR2 | RAS (SEQ ID NO: 2603) |
| (IMGT) | |
| LCDR3 | FQYRHMPSQT (SEQ ID NO: 2601) |
| (IMGT) | |
| VL | DIQMTQSPSSLSASVGDRVTITCRASQDISNYLAWYQQKPGKAPKLLIYRASSL |
| | QSGVPSRFSGSGSGTDFTLTISSLQPEDFATYYCFQYRHMPSQTFGQGTKVEIK |
| | (SEQ ID NO: 2606) |
| DNA VL | GACATTCAGATGACCCAGTCCCCGTCGTCCCTGTCCGCATCCGTGGGCGAC |
| | AGAGTCACCATCACTTGCCGGGCCTCACAGGATATTTCCAACTACCTGGCC |
| | TGGTATCAGCAGAAGCCTGGAAAGGCCCCGAAGCTGCTGATCTACCGGGC |
| | GTCCTCCTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCTTCCAGTACCGGCACATGCCCTCACAAACCTTCGGACAGGG |
| | CACCAAAGTCGAGATCAAG (SEQ ID NO: 2613) |
| Light Chain | DIQMTQSPSSLSASVGDRVTITCRASQDISNYLAWYQQKPGKAPKLLIYRASSL |
| | QSGVPSRFSGSGSGTDFTLTISSLQPEDFATYYCFQYRHMPSQTFGQGTKVEIK |
| | RTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGE |
| | C (SEQ ID NO: 2608) |
| DNA Light | GACATTCAGATGACCCAGTCCCCGTCGTCCCTGTCCGCATCCGTGGGCGAC |
| Chain | AGAGTCACCATCACTTGCCGGGCCTCACAGGATATTTCCAACTACCTGGCC |
| | TGGTATCAGCAGAAGCCTGGAAAGGCCCCGAAGCTGCTGATCTACCGGGC |
| | GTCCTCCTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCTTCCAGTACCGGCACATGCCCTCACAAACCTTCGGACAGGG |
| | CACCAAAGTCGAGATCAAGCGTACGGTGGCCGCTCCCAGCGTGTTCATCTT |
| | CCCCCCCAGCGACGAGCAGCTGAAGAGCGGCACCGCCAGCGTGGTGTGCC |
| | TGCTGAACAACTTCTACCCCCGGGAGGCCAAGGTGCAGTGGAAGGTGGAC |
| | AACGCCCTGCAGAGCGGCAACAGCCAGGAGAGCGTCACCGAGCAGGACA |
| | GCAAGGACTCCACCTACAGCCTGAGCAGCACCCTGACCCTGAGCAAGGCC |
| | GACTACGAGAAGCATAAGGTGTACGCCTGCGAGGTGACCCACCAGGGCCT |
| | GTCCAGCCCCGTGACCAAGAGCTTCAACAGGGGCGAGTGC (SEQ ID NO: 2614) |

MOR44698C

| | |
|---|---|
| HCDR1 | GYTFTGYHMS (SEQ ID NO: 2586) |
| (Combined) | |
| HCDR2 | VINPVSGNTVYAQKFQG (SEQ ID NO: 2587) |
| (Combined) | |
| HCDR3 | IPSYTYAFDY (SEQ ID NO: 2588) |
| (Combined) | |
| HCDR1 | GYHMS (SEQ ID NO: 2589) |
| (Kabat) | |
| HCDR2 | VINPVSGNTVYAQKFQG (SEQ ID NO: 2587) |
| (Kabat) | |
| HCDR3 | IPSYTYAFDY (SEQ ID NO: 2588) |
| (Kabat) | |
| HCDR1 | GYTFTGY (SEQ ID NO: 2590) |
| (Chothia) | |
| HCDR2 | NPVSGN (SEQ ID NO: 2591) |
| (Chothia) | |
| HCDR3 | IPSYTYAFDY (SEQ ID NO: 2588) |
| (Chothia) | |
| HCDR1 | GYTFTGYH (SEQ ID NO: 2592) |
| (IMGT) | |
| HCDR2 | INPVSGNT (SEQ ID NO: 2593) |
| (IMGT) | |
| HCDR3 | ARIPSYTYAFDY (SEQ ID NO: 2594) |
| (IMGT) | |
| VH | QVQLVQSGAEVKKPGASVKVSCKASGYTFTGYHMSWVRQAPGQGLEWMGV |
| | INPVSGNTVYAQKFQGRVTMTRDTSISTAYMELSRLRSEDTAVYYCARIPSYT |
| | YAFDYWGQGTLVTVSS (SEQ ID NO: 2595) |
| DNA VH | CAAGTGCAACTCGTGCAGTCAGGAGCCGAAGTCAAGAAGCCTGGAGCCTC |
| | GGTCAAGGTGTCCTGCAAGGCCAGCGGATACACTTTCACTGGATACCACAT |
| | GTCGTGGGTCAGACAGGCTCCTGGCCAAGGGCTGGAGTGGATGGGCGTCA |
| | TCAACCCGGTGTCGGGTAATACCGTGTACGCCCAGAAGTTCCAGGGTCGCG |
| | TGACCATGACCCGGGATACCTCCATTAGCACCGCGTACATGGAGCTCAGCC |
| | GGTTGAGATCCGAGGATACCGCCGTGTACTACTGTGCGCGGATCCCGTCCT |
| | ACACTTACGCCTTCGACTATTGGGGCCAGGGGACTCTTGTCACCGTGTCCT |
| | CG (SEQ ID NO: 2610) |
| Heavy Chain | QVQLVQSGAEVKKPGASVKVSCKASGYTFTGYHMSWVRQAPGQGLEWMGV |
| | INPVSGNTVYAQKFQGRVTMTRDTSISTAYMELSRLRSEDTAVYYCARIPSYT |
| | YAFDYWGQGTLVTVSSASTKGPSVFPLAPSSKSTSGGTAALGCLVKDYFPEPV |
| | TVSWNSGALTSGVHTFPAVLQSSGLYSLSSVVTVPSSSLGTQTYICNVNHKPSN |
| | TKVDKRVEPKSCDKTHTCPPCPAPELLGGPSVFLFPPKPKDTLMISRTPEVTCV |
| | VVAVSHEDPEVKFNWYVDGVEVHNAKTKPREEQYNSTYRVVSVLTVLHQD |
| | WLNGKEYKCKVSNKALAAPIEKTISKAKGQPREPQVYTLPPSREEMTKNQVSL |
| | TCLVKGFYPSDIAVEWESNGQPENNYKTTPPVLDSDGSFFLYSKLTVDKSRWQ |
| | QGNVFSCSVMHEALHNHYTQKSLSLSPGK (SEQ ID NO: 2615) |
| DNA Heavy | CAAGTGCAACTCGTGCAGTCAGGAGCCGAAGTCAAGAAGCCTGGAGCCTC |
| Chain | GGTCAAGGTGTCCTGCAAGGCCAGCGGATACACTTTCACTGGATACCACAT |
| | GTCGTGGGTCAGACAGGCTCCTGGCCAAGGGCTGGAGTGGATGGGCGTCA |
| | TCAACCCGGTGTCGGGTAATACCGTGTACGCCCAGAAGTTCCAGGGTCGCG |
| | TGACCATGACCCGGGATACCTCCATTAGCACCGCGTACATGGAGCTCAGCC |
| | GGTTGAGATCCGAGGATACCGCCGTGTACTACTGTGCGCGGATCCCGTCCT |
| | ACACTTACGCCTTCGACTATTGGGGCCAGGGGACTCTTGTCACCGTGTCCT |
| | CGGCCTCCACTAAGGGCCCGTCAGTGTTCCCCCTTGCGCCATCCTCGAAGT |
| | CAACCTCCGGAGGAACTGCCGCACTGGGTTGCCTCGTGAAAGACTATTTCC |
| | CGGAACCCGTCACTGTCTCCTGGAACTCAGGAGCGCTCACCAGCGGAGTGC |
| | ATACCTTTCCTGCGGTGCTGCAGTCCAGCGGCCTGTACTCCCTGAGCTCCGT |
| | CGTGACCGTCCCCTCGTCGTCCCTGGGAACCCAAACCTACATTTGCAACGT |
| | CAATCACAAGCCAAGCAACACTAAGGTGGACAAGAGAGTGGAGCCCAAGT |
| | CCTGCGATAAGACCCACACCTGTCCTCCCTGTCCGGCACCTGAACTGCTTG |
| | GTGGACCTTCCGTGTTCCTGTTCCCGCCCAAGCCAAAAGACACCCTGATGA |
| | TCTCCCGCACTCCGGAAGTCACTTGCGTGGTCGTGGCCGTGTCCCACGAGG |
| | ACCCCGAGGTCAAGTTTAATTGGTACGTGGACGGAGTGGAAGTGCACAAC |
| | GCCAAGACCAAGCCGCGGGAAGAACAGTACAACTCCACCTACCGCGTGGT |
| | GTCCGTCCTGACTGTGCTCCACCAGGACTGGCTGAACGGAAAGGAGTACA |
| | AGTGCAAAGTGTCCAACAAGGCACTGGCTGCCCCTATCGAAAAGACTATCT |
| | CCAAGGCCAAGGGCCAACCTAGGGAGCCCCAGGTGTACACGTTGCCTCCTT |
| | CCCGCGAAGAAATGACTAAGAACCAGGTGTCGCTGACCTGTCTCGTGAAA |
| | GGGTTCTACCCCTCTGACATCGCCGTGGAATGGGAGTCAAACGGACAGCCT |
| | GAGAACAACTATAAGACCACACCACCTGTCCTGGACTCCGACGGCTCCTTC |
| | TTCCTGTACTCAAAGTTGACCGTGGACAAGTCGCGGTGGCAACAGGGCAAC |
| | GTGTTCTCTTGCTCCGTGATGCACGAAGCCCTGCACAACCACTACACCCAA |
| | AAGTCGCTCAGCCTCTCCCCCGGAAAG (SEQ ID NO: 2616) |
| LCDR1 | RASQDISNYLA (SEQ ID NO: 2599) |
| (Combined) | |
| LCDR2 | RASSLQS (SEQ ID NO: 2600) |
| (Combined) | |
| LCDR3 | FQYRHMPSQT (SEQ ID NO: 2601) |
| (Combined) | |
| LCDR1 | RASQDISNYLA (SEQ ID NO: 2599) |
| (Kabat) | |
| LCDR2 | RASSLQS (SEQ ID NO: 2600) |
| (Kabat) | |
| LCDR3 | FQYRHMPSQT (SEQ ID NO: 2601) |
| (Kabat) | |
| LCDR1 | SQDISNY (SEQ ID NO: 2602) |
| (Chothia) | |
| LCDR2 | RAS (SEQ ID NO: 2603) |
| (Chothia) | |
| LCDR3 | YRHMPSQ (SEQ ID NO: 2604) |
| (Chothia) | |
| LCDR1 | QDISNY (SEQ ID NO: 2605) |
| (IMGT) | |
| LCDR2 | RAS (SEQ ID NO: 2603) |
| (IMGT) | |
| LCDR3 | FQYRHMPSQT (SEQ ID NO: 2601) |
| (IMGT) | |
| VL | DIQMTQSPSSLSASVGDRVTITCRASQDISNYLAWYQQKPGKAPKLLIYRASSL |
| | QSGVPSRFSGSGSGTDFTLTISSLQPEDFATYYCFQYRHMPSQTFGQGTKVEIK |
| | (SEQ ID NO: 2606) |
| DNA VL | GACATTCAGATGACCCAGTCCCCGTCGTCCCTGTCCGCATCCGTGGGCGAC |
| | AGAGTCACCATCACTTGCCGGGCCTCACAGGATATTTCCAACTACCTGGCC |
| | TGGTATCAGCAGAAGCCTGGAAAGGCCCCGAAGCTGCTGATCTACCGGGC |
| | GTCCTCCTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCTTCCAGTACCGGCACATGCCCTCACAAACCTTCGGACAGGG |
| | CACCAAAGTCGAGATCAAG (SEQ ID NO: 2613) |
| Light Chain | DIQMTQSPSSLSASVGDRVTITCRASQDISNYLAWYQQKPGKAPKLLIYRASSL |
| | QSGVPSRFSGSGSGTDFTLTISSLQPEDFATYYCFQYRHMPSQTFGQGTKVEIK |
| | RTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGE |
| | C (SEQ ID NO: 2608) |
| DNA Light | GACATTCAGATGACCCAGTCCCCGTCGTCCCTGTCCGCATCCGTGGGCGAC |
| Chain | AGAGTCACCATCACTTGCCGGGCCTCACAGGATATTTCCAACTACCTGGCC |
| | TGGTATCAGCAGAAGCCTGGAAAGGCCCCGAAGCTGCTGATCTACCGGGC |
| | GTCCTCCTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCTTCCAGTACCGGCACATGCCCTCACAAACCTTCGGACAGGG |
| | CACCAAAGTCGAGATCAAGCGTACGGTGGCCGCTCCCAGCGTGTTCATCTT |
| | CCCCCCCAGCGACGAGCAGCTGAAGAGCGGCACCGCCAGCGTGGTGTGCC |
| | TGCTGAACAACTTCTACCCCCGGGAGGCCAAGGTGCAGTGGAAGGTGGAC |
| | AACGCCCTGCAGAGCGGCAACAGCCAGGAGAGCGTCACCGAGCAGGACA |
| | GCAAGGACTCCACCTACAGCCTGAGCAGCACCCTGACCCTGAGCAAGGCC |
| | GACTACGAGAAGCATAAGGTGTACGCCTGCGAGGTGACCCACCAGGGCCT |
| | GTCCAGCCCCGTGACCAAGAGCTTCAACAGGGGCGAGTGC (SEQ ID NO: 2614) |

MOR44698D

| | |
|---|---|
| HCDR1 | GYTFTGYHMS (SEQ ID NO: 2586) |
| (Combined) | |
| HCDR2 | VINPVSGNTVYAQKFQG (SEQ ID NO: 2587) |
| (Combined) | |
| HCDR3 | IPSYTYAFDY (SEQ ID NO: 2588) |
| (Combined) | |
| HCDR1 | GYHMS (SEQ ID NO: 2589) |
| (Kabat) | |
| HCDR2 | VINPVSGNTVYAQKFQG (SEQ ID NO: 2587) |
| (Kabat) | |
| HCDR3 | IPSYTYAFDY (SEQ ID NO: 2588) |
| (Kabat) | |
| HCDR1 | GYTFTGY (SEQ ID NO: 2590) |
| (Chothia) | |
| HCDR2 | NPVSGN (SEQ ID NO: 2591) |
| (Chothia) | |
| HCDR3 | IPSYTYAFDY (SEQ ID NO: 2588) |
| (Chothia) | |
| HCDR1 | GYTFTGYH (SEQ ID NO: 2592) |
| (IMGT) | |
| HCDR2 | INPVSGNT (SEQ ID NO: 2593) |
| (IMGT) | |
| HCDR3 | ARIPSYTYAFDY (SEQ ID NO: 2594) |
| (IMGT) | |
| VH | QVQLVQSGAEVKKPGASVKVSCKASGYTFTGYHMSWVRQAPGQGLEWMGV |
| | INPVSGNTVYAQKFQGRVTMTRDTSISTAYMELSRLRSEDTAVYYCARIPSYT |
| | YAFDYWGQGTLVTVSS (SEQ ID NO: 2595) |
| DNA VH | CAAGTGCAACTCGTGCAGTCAGGAGCCGAAGTCAAGAAGCCTGGAGCCTC |
| | GGTCAAGGTGTCCTGCAAGGCCAGCGGATACACTTTCACTGGATACCACAT |
| | GTCGTGGGTCAGACAGGCTCCTGGCCAAGGGCTGGAGTGGATGGGCGTCA |
| | TCAACCCGGTGTCGGGTAATACCGTGTACGCCCAGAAGTTCCAGGGTCGCG |
| | TGACCATGACCCGGGATACCTCCATTAGCACCGCGTACATGGAGCTCAGCC |
| | GGTTGAGATCCGAGGATACCGCCGTGTACTACTGTGCGCGGATCCCGTCCT |
| | ACACTTACGCCTTCGACTATTGGGGCCAGGGGACTCTTGTCACCGTGTCCT |
| | CG (SEQ ID NO: 2610) |
| Heavy Chain | QVQLVQSGAEVKKPGASVKVSCKASGYTFTGYHMSWVRQAPGQGLEWMGV |
| | INPVSGNTVYAQKFQGRVTMTRDTSISTAYMELSRLRSEDTAVYYCARIPSYT |
| | YAFDYWGQGTLVTVSSASTKGPSVFPLAPSSKSTSGGTAALGCLVKDYFPEPV |
| | TVSWNSGALTSGVHTFPAVLQSSGLYSLSSVVTVPSSSLGTQTYICNVNHKPSN |
| | TKVDKRVEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSREEMTKNQVSL |
| | TCLVKGFYPSDIAVEWESNGQPENNYKTTPPVLDSDGSFFLYSKLTVDKSRWQ |
| | QGNVFSCSVLHEALHSHYTQKSLSLSPGK (SEQ ID NO: 2617) |
| DNA Heavy | CAAGTGCAACTCGTGCAGTCAGGAGCCGAAGTCAAGAAGCCTGGAGCCTC |
| Chain | GGTCAAGGTGTCCTGCAAGGCCAGCGGATACACTTTCACTGGATACCACAT |
| | GTCGTGGGTCAGACAGGCTCCTGGCCAAGGGCTGGAGTGGATGGGCGTCA |
| | TCAACCCGGTGTCGGGTAATACCGTGTACGCCCAGAAGTTCCAGGGTCGCG |
| | TGACCATGACCCGGGATACCTCCATTAGCACCGCGTACATGGAGCTCAGCC |
| | GGTTGAGATCCGAGGATACCGCCGTGTACTACTGTGCGCGGATCCCGTCCT |
| | ACACTTACGCCTTCGACTATTGGGGCCAGGGGACTCTTGTCACCGTGTCCT |
| | CGGCCTCCACTAAGGGCCCGTCAGTGTTCCCCCTTGCGCCATCCTCGAAGT |
| | CAACCTCCGGAGGAACTGCCGCACTGGGTTGCCTCGTGAAAGACTATTTCC |
| | CGGAACCCGTCACTGTCTCCTGGAACTCAGGAGCGCTCACCAGCGGAGTGC |
| | ATACCTTTCCTGCGGTGCTGCAGTCCAGCGGCCTGTACTCCCTGAGCTCCGT |
| | CGTGACCGTCCCCTCGTCGTCCCTGGGAACCCAAACCTACATTTGCAACGT |
| | CAATCACAAGCCAAGCAACACTAAGGTGGACAAGAGAGTGGAGCCCAAGT |
| | CCTGCGATAAGACCCACACCTGTCCTCCCTGTCCGGCACCTGAACTGCTTG |
| | GTGGACCTTCCGTGTTCCTGTTCCCGCCCAAGCCAAAAGACACCCTGATGA |
| | TCTCCCGCACTCCGGAAGTCACTTGCGTGGTCGTGGACGTGTCCCACGAGG |
| | ACCCCGAGGTCAAGTTTAATTGGTACGTGGACGGAGTGGAAGTGCACAAC |
| | GCCAAGACCAAGCCGCGGGAAGAACAGTACAACTCCACCTACCGCGTGGT |
| | GTCCGTCCTGACTGTGCTCCACCAGGACTGGCTGAACGGAAAGGAGTACA |
| | AGTGCAAAGTGTCCAACAAGGCACTGCCAGCCCCTATCGAAAAGACTATC |
| | TCCAAGGCCAAGGGCCAACCTAGGGAGCCCCAGGTGTACACGTTGCCTCCT |
| | TCCCGCGAAGAAATGACTAAGAACCAGGTGTCGCTGACCTGTCTCGTGAAA |
| | GGGTTCTACCCCTCTGACATCGCCGTGGAATGGGAGTCAAACGGACAGCCT |
| | GAGAACAACTATAAGACCACACCACCTGTCCTGGACTCCGACGGCTCCTTC |
| | TTCCTGTACTCAAAGTTGACCGTGGACAAGTCGCGGTGGCAACAGGGCAAC |
| | GTGTTCTCTTGCTCCGTGCTGCACGAAGCCCTGCACAGCCACTACACCCAA |
| | AAGTCGCTCAGCCTCTCCCCCGGAAAG (SEQ ID NO: 2618) |
| LCDR1 | RASQDISNYLA (SEQ ID NO: 2599) |
| (Combined) | |
| LCDR2 | RASSLQS (SEQ ID NO: 2600) |
| (Combined) | |
| LCDR3 | FQYRHMPSQT (SEQ ID NO: 2601) |
| (Combined) | |
| LCDR1 | RASQDISNYLA (SEQ ID NO: 2599) |
| (Kabat) | |
| LCDR2 | RASSLQS (SEQ ID NO: 2600) |
| (Kabat) | |
| LCDR3 | FQYRHMPSQT (SEQ ID NO: 2601) |
| (Kabat) | |
| LCDR1 | SQDISNY (SEQ ID NO: 2602) |
| (Chothia) | |
| LCDR2 | RAS (SEQ ID NO: 2603) |
| (Chothia) | |
| LCDR3 | YRHMPSQ (SEQ ID NO: 2604) |
| (Chothia) | |
| LCDR1 | QDISNY (SEQ ID NO: 2605) |
| (IMGT) | |
| LCDR2 | RAS (SEQ ID NO: 2603) |
| (IMGT) | |
| LCDR3 | FQYRHMPSQT (SEQ ID NO: 2601) |
| (IMGT) | |
| VL | DIQMTQSPSSLSASVGDRVTITCRASQDISNYLAWYQQKPGKAPKLLIYRASSL |
| | QSGVPSRFSGSGSGTDFTLTISSLQPEDFATYYCFQYRHMPSQTFGQGTKVEIK |
| | (SEQ ID NO: 2606) |
| DNA VL | GACATTCAGATGACCCAGTCCCCGTCGTCCCTGTCCGCATCCGTGGGCGAC |
| | AGAGTCACCATCACTTGCCGGGCCTCACAGGATATTTCCAACTACCTGGCC |
| | TGGTATCAGCAGAAGCCTGGAAAGGCCCCGAAGCTGCTGATCTACCGGGC |
| | GTCCTCCTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCTTCCAGTACCGGCACATGCCCTCACAAACCTTCGGACAGGG |
| | CACCAAAGTCGAGATCAAG (SEQ ID NO: 2613) |
| Light Chain | DIQMTQSPSSLSASVGDRVTITCRASQDISNYLAWYQQKPGKAPKLLIYRASSL |
| | QSGVPSRFSGSGSGTDFTLTISSLQPEDFATYYCFQYRHMPSQTFGQGTKVEIK |
| | RTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGE |
| | C (SEQ ID NO: 2608) |
| DNA Light | GACATTCAGATGACCCAGTCCCCGTCGTCCCTGTCCGCATCCGTGGGCGAC |
| Chain | AGAGTCACCATCACTTGCCGGGCCTCACAGGATATTTCCAACTACCTGGCC |
| | TGGTATCAGCAGAAGCCTGGAAAGGCCCCGAAGCTGCTGATCTACCGGGC |
| | GTCCTCCTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCTTCCAGTACCGGCACATGCCCTCACAAACCTTCGGACAGGG |
| | CACCAAAGTCGAGATCAAGCGTACGGTGGCCGCTCCCAGCGTGTTCATCTT |
| | CCCCCCCAGCGACGAGCAGCTGAAGAGCGGCACCGCCAGCGTGGTGTGCC |
| | TGCTGAACAACTTCTACCCCCGGGAGGCCAAGGTGCAGTGGAAGGTGGAC |
| | AACGCCCTGCAGAGCGGCAACAGCCAGGAGAGCGTCACCGAGCAGGACA |
| | GCAAGGACTCCACCTACAGCCTGAGCAGCACCCTGACCCTGAGCAAGGCC |
| | GACTACGAGAAGCATAAGGTGTACGCCTGCGAGGTGACCCACCAGGGCCT |
| | GTCCAGCCCCGTGACCAAGAGCTTCAACAGGGGCGAGTGC (SEQ ID NO: 2614) |

MOR44698E

| | |
|---|---|
| HCDR1 | GYTFTGYHMS (SEQ ID NO: 2586) |
| (Combined) | |
| HCDR2 | VINPVSGNTVYAQKFQG (SEQ ID NO: 2587) |
| (Combined) | |
| HCDR3 | IPSYTYAFDY (SEQ ID NO: 2588) |
| (Combined) | |
| HCDR1 | GYHMS (SEQ ID NO: 2589) |
| (Kabat) | |
| HCDR2 | VINPVSGNTVYAQKFQG (SEQ ID NO: 2587) |
| (Kabat) | |
| HCDR3 | IPSYTYAFDY (SEQ ID NO: 2588) |
| (Kabat) | |
| HCDR1 | GYTFTGY (SEQ ID NO: 2590) |
| (Chothia) | |
| HCDR2 | NPVSGN (SEQ ID NO: 2591) |
| (Chothia) | |
| HCDR3 | IPSYTYAFDY (SEQ ID NO: 2588) |
| (Chothia) | |
| HCDR1 | GYTFTGYH (SEQ ID NO: 2592) |
| (IMGT) | |
| HCDR2 | INPVSGNT (SEQ ID NO: 2593) |
| (IMGT) | |
| HCDR3 | ARIPSYTYAFDY (SEQ ID NO: 2594) |
| (IMGT) | |
| VH | QVQLVQSGAEVKKPGASVKVSCKASGYTFTGYHMSWVRQAPGQGLEWMGV |
| | INPVSGNTVYAQKFQGRVTMTRDTSISTAYMELSRLRSEDTAVYYCARIPSYT |
| | YAFDYWGQGTLVTVSS (SEQ ID NO: 2595) |
| DNA VH | CAAGTGCAACTCGTGCAGTCAGGAGCCGAAGTCAAGAAGCCTGGAGCCTC |
| | GGTCAAGGTGTCCTGCAAGGCCAGCGGATACACTTTCACTGGATACCACAT |
| | GTCGTGGGTCAGACAGGCTCCTGGCCAAGGGCTGGAGTGGATGGGCGTCA |
| | TCAACCCGGTGTCGGGTAATACCGTGTACGCCCAGAAGTTCCAGGGTCGCG |
| | TGACCATGACCCGGGATACCTCCATTAGCACCGCGTACATGGAGCTCAGCC |
| | GGTTGAGATCCGAGGATACCGCCGTGTACTACTGTGCGCGGATCCCGTCCT |
| | ACACTTACGCCTTCGACTATTGGGGCCAGGGGACTCTTGTCACCGTGTCCT |
| | CG (SEQ ID NO: 2610) |
| Heavy Chain | QVQLVQSGAEVKKPGASVKVSCKASGYTFTGYHMSWVRQAPGQGLEWMGV |
| | INPVSGNTVYAQKFQGRVTMTRDTSISTAYMELSRLRSEDTAVYYCARIPSYT |
| | YAFDYWGQGTLVTVSSASTKGPSVFPLAPSSKSTSGGTAALGCLVKDYFPEPV |
| | TVSWNSGALTSGVHTFPAVLQSSGLYSLSSVVTVPSSSLGTQTYICNVNHKPSN |
| | TKVDKRVEPKSCDKTHTCPPCPAPELLGGPSVFLFPPKPKDTLMISRTPEVTCV |
| | VVAVSHEDPEVKFNWYVDGVEVHNAKTKPREEQYNSTYRVVSVLTVLHQD |
| | WLNGKEYKCKVSNKALAAPIEKTISKAKGQPREPQVYTLPPSREEMTKNQVSL |
| | TCLVKGFYPSDIAVEWESNGQPENNYKTTPPVLDSDGSFFLYSKLTVDKSRWQ |
| | QGNVFSCSVLHEALHSHYTQKSLSLSPGK (SEQ ID NO: 2619) |
| DNA Heavy | CAAGTGCAACTCGTGCAGTCAGGAGCCGAAGTCAAGAAGCCTGGAGCCTC |
| Chain | GGTCAAGGTGTCCTGCAAGGCCAGCGGATACACTTTCACTGGATACCACAT |
| | GTCGTGGGTCAGACAGGCTCCTGGCCAAGGGCTGGAGTGGATGGGCGTCA |
| | TCAACCCGGTGTCGGGTAATACCGTGTACGCCCAGAAGTTCCAGGGTCGCG |
| | TGACCATGACCCGGGATACCTCCATTAGCACCGCGTACATGGAGCTCAGCC |
| | GGTTGAGATCCGAGGATACCGCCGTGTACTACTGTGCGCGGATCCCGTCCT |
| | ACACTTACGCCTTCGACTATTGGGGCCAGGGGACTCTTGTCACCGTGTCCT |
| | CGGCCTCCACTAAGGGCCCGTCAGTGTTCCCCCTTGCGCCATCCTCGAAGT |
| | CAACCTCCGGAGGAACTGCCGCACTGGGTTGCCTCGTGAAAGACTATTTCC |
| | CGGAACCCGTCACTGTCTCCTGGAACTCAGGAGCGCTCACCAGCGGAGTGC |
| | ATACCTTTCCTGCGGTGCTGCAGTCCAGCGGCCTGTACTCCCTGAGCTCCGT |
| | CGTGACCGTCCCCTCGTCGTCCCTGGGAACCCAAACCTACATTTGCAACGT |
| | CAATCACAAGCCAAGCAACACTAAGGTGGACAAGAGAGTGGAGCCCAAGT |
| | CCTGCGATAAGACCCACACCTGTCCTCCCTGTCCGGCACCTGAACTGCTTG |
| | GTGGACCTTCCGTGTTCCTGTTCCCGCCCAAGCCAAAAGACACCCTGATGA |
| | TCTCCCGCACTCCGGAAGTCACTTGCGTGGTCGTGGCCGTGTCCCACGAGG |
| | ACCCCGAGGTCAAGTTTAATTGGTACGTGGACGGAGTGGAAGTGCACAAC |
| | GCCAAGACCAAGCCGCGGGAAGAACAGTACAACTCCACCTACCGCGTGGT |
| | GTCCGTCCTGACTGTGCTCCACCAGGACTGGCTGAACGGAAAGGAGTACA |
| | AGTGCAAAGTGTCCAACAAGGCACTGGCTGCCCCTATCGAAAAGACTATCT |
| | CCAAGGCCAAGGGCCAACCTAGGGAGCCCCAGGTGTACACGTTGCCTCCTT |
| | CCCGCGAAGAAATGACTAAGAACCAGGTGTCGCTGACCTGTCTCGTGAAA |
| | GGGTTCTACCCCTCTGACATCGCCGTGGAATGGGAGTCAAACGGACAGCCT |
| | GAGAACAACTATAAGACCACACCACCTGTCCTGGACTCCGACGGCTCCTTC |
| | TTCCTGTACTCAAAGTTGACCGTGGACAAGTCGCGGTGGCAACAGGGCAAC |
| | GTGTTCTCTTGCTCCGTGCTGCACGAAGCCCTGCACAGCCACTACACCCAA |
| | AAGTCGCTCAGCCTCTCCCCCGGAAAG (SEQ ID NO: 2620) |
| LCDR1 | RASQDISNYLA (SEQ ID NO: 2599) |
| (Combined) | |
| LCDR2 | RASSLQS (SEQ ID NO: 2600) |
| (Combined) | |
| LCDR3 | FQYRHMPSQT (SEQ ID NO: 2601) |
| (Combined) | |
| LCDR1 | RASQDISNYLA (SEQ ID NO: 2599) |
| (Kabat) | |
| LCDR2 | RASSLQS (SEQ ID NO: 2600) |
| (Kabat) | |
| LCDR3 | FQYRHMPSQT (SEQ ID NO: 2601) |
| (Kabat) | |
| LCDR1 | SQDISNY (SEQ ID NO: 2602) |
| (Chothia) | |
| LCDR2 | RAS (SEQ ID NO: 2603) |
| (Chothia) | |
| LCDR3 | YRHMPSQ (SEQ ID NO: 2604) |
| (Chothia) | |
| LCDR1 | QDISNY (SEQ ID NO: 2605) |
| (IMGT) | |
| LCDR2 | RAS (SEQ ID NO: 2603) |
| (IMGT) | |
| LCDR3 | FQYRHMPSQT (SEQ ID NO: 2601) |
| (IMGT) | |
| VL | DIQMTQSPSSLSASVGDRVTITCRASQDISNYLAWYQQKPGKAPKLLIYRASSL |
| | QSGVPSRFSGSGSGTDFTLTISSLQPEDFATYYCFQYRHMPSQTFGQGTKVEIK |
| | (SEQ ID NO: 2606) |
| DNA VL | GACATTCAGATGACCCAGTCCCCGTCGTCCCTGTCCGCATCCGTGGGCGAC |
| | AGAGTCACCATCACTTGCCGGGCCTCACAGGATATTTCCAACTACCTGGCC |
| | TGGTATCAGCAGAAGCCTGGAAAGGCCCCGAAGCTGCTGATCTACCGGGC |
| | GTCCTCCTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCTTCCAGTACCGGCACATGCCCTCACAAACCTTCGGACAGGG |
| | CACCAAAGTCGAGATCAAG (SEQ ID NO: 2613) |
| Light Chain | DIQMTQSPSSLSASVGDRVTITCRASQDISNYLAWYQQKPGKAPKLLIYRASSL |
| | QSGVPSRFSGSGSGTDFTLTISSLQPEDFATYYCFQYRHMPSQTFGQGTKVEIK |
| | RTVAAPSVFIFPP SDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGE |
| | C (SEQ ID NO: 2608) |
| DNA Light | GACATTCAGATGACCCAGTCCCCGTCGTCCCTGTCCGCATCCGTGGGCGAC |
| Chain | AGAGTCACCATCACTTGCCGGGCCTCACAGGATATTTCCAACTACCTGGCC |
| | TGGTATCAGCAGAAGCCTGGAAAGGCCCCGAAGCTGCTGATCTACCGGGC |
| | GTCCTCCTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCTTCCAGTACCGGCACATGCCCTCACAAACCTTCGGACAGGG |
| | CACCAAAGTCGAGATCAAGCGTACGGTGGCCGCTCCCAGCGTGTTCATCTT |
| | CCCCCCCAGCGACGAGCAGCTGAAGAGCGGCACCGCCAGCGTGGTGTGCC |
| | TGCTGAACAACTTCTACCCCCGGGAGGCCAAGGTGCAGTGGAAGGTGGAC |
| | AACGCCCTGCAGAGCGGCAACAGCCAGGAGAGCGTCACCGAGCAGGACA |
| | GCAAGGACTCCACCTACAGCCTGAGCAGCACCCTGACCCTGAGCAAGGCC |
| | GACTACGAGAAGCATAAGGTGTACGCCTGCGAGGTGACCCACCAGGGCCT |
| | GTCCAGCCCCGTGACCAAGAGCTTCAACAGGGGCGAGTGC (SEQ ID NO: 2614) |

MOR44698F

| | |
|---|---|
| HCDR1 | GYTFTGYHMS (SEQ ID NO: 2586) |
| (Combined) | |
| HCDR2 | VINPVSGNTVYAQKFQG (SEQ ID NO: 2587) |
| (Combined) | |
| HCDR3 | IPSYTYAFDY (SEQ ID NO: 2588) |
| (Combined) | |
| HCDR1 | GYHMS (SEQ ID NO: 2589) |
| (Kabat) | |
| HCDR2 | VINPVSGNTVYAQKFQG (SEQ ID NO: 2587) |
| (Kabat) | |
| HCDR3 | IPSYTYAFDY (SEQ ID NO: 2588) |
| (Kabat) | |
| HCDR1 | GYTFTGY (SEQ ID NO: 2590) |
| (Chothia) | |
| HCDR2 | NPVSGN (SEQ ID NO: 2591) |
| (Chothia) | |
| HCDR3 | IPSYTYAFDY (SEQ ID NO: 2588) |
| (Chothia) | |
| HCDR1 | GYTFTGYH (SEQ ID NO: 2592) |
| (IMGT) | |
| HCDR2 | INPVSGNT (SEQ ID NO: 2593) |
| (IMGT) | |
| HCDR3 | ARIPSYTYAFDY (SEQ ID NO: 2594) |
| (IMGT) | |
| VH | QVQLVQSGAEVKKPGASVKVSCKASGYTFTGYHMSWVRQAPGQGLEWMGV |
| | INPVSGNTVYAQKFQGRVTMTRDTSISTAYMELSRLRSEDTAVYYCARIPSYT |
| | YAFDYWGQGTLVTVSS (SEQ ID NO: 2595) |
| DNA VH | CAAGTGCAACTCGTGCAGTCAGGAGCCGAAGTCAAGAAGCCTGGAGCCTC |
| | GGTCAAGGTGTCCTGCAAGGCCAGCGGATACACTTTCACTGGATACCACAT |
| | GTCGTGGGTCAGACAGGCTCCTGGCCAAGGGCTGGAGTGGATGGGCGTCA |
| | TCAACCCGGTGTCGGGTAATACCGTGTACGCCCAGAAGTTCCAGGGTCGCG |
| | TGACCATGACCCGGGATACCTCCATTAGCACCGCGTACATGGAGCTCAGCC |
| | GGTTGAGATCCGAGGATACCGCCGTGTACTACTGTGCGCGGATCCCGTCCT |
| | ACACTTACGCCTTCGACTATTGGGGCCAGGGGACTCTTGTCACCGTGTCCT |
| | CG (SEQ ID NO: 2610) |
| Heavy Chain | QVQLVQSGAEVKKPGASVKVSCKASGYTFTGYHMSWVRQAPGQGLEWMGV |
| | INPVSGNTVYAQKFQGRVTMTRDTSISTAYMELSRLRSEDTAVYYCARIPSYT |
| | YAFDYWGQGTLVTVSSASTKGPSVFPLAPSSKSTSGGTAALGCLVKDYFPEPV |
| | TVSWNSGALTSGVHTFPAVLQSSGLYSLSSVVTVPSSSLGTQTYICNVNHKPSN |
| | TKVDKRVEPKSCDKTHTCPPCPAPELLGGPSVFLFPPKPKDTLYITREPEVTCV |
| | VVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSREEMTKNQVSL |
| | TCLVKGFYPSDIAVEWESNGQPENNYKTTPPVLDSDGSFFLYSKLTVDKSRWQ |
| | QGNVFSCSVMHEALHNHYTQKSLSLSPGK (SEQ ID NO: 2621) |
| DNA Heavy | CAAGTGCAACTCGTGCAGTCAGGAGCCGAAGTCAAGAAGCCTGGAGCCTC |
| Chain | GGTCAAGGTGTCCTGCAAGGCCAGCGGATACACTTTCACTGGATACCACAT |
| | GTCGTGGGTCAGACAGGCTCCTGGCCAAGGGCTGGAGTGGATGGGCGTCA |
| | TCAACCCGGTGTCGGGTAATACCGTGTACGCCCAGAAGTTCCAGGGTCGCG |
| | TGACCATGACCCGGGATACCTCCATTAGCACCGCGTACATGGAGCTCAGCC |
| | GGTTGAGATCCGAGGATACCGCCGTGTACTACTGTGCGCGGATCCCGTCCT |
| | ACACTTACGCCTTCGACTATTGGGGCCAGGGGACTCTTGTCACCGTGTCCT |
| | CGGCCTCCACTAAGGGCCCGTCAGTGTTCCCCCTTGCGCCATCCTCGAAGT |
| | CAACCTCCGGAGGAACTGCCGCACTGGGTTGCCTCGTGAAAGACTATTTCC |
| | CGGAACCCGTCACTGTCTCCTGGAACTCAGGAGCGCTCACCAGCGGAGTGC |
| | ATACCTTTCCTGCGGTGCTGCAGTCCAGCGGCCTGTACTCCCTGAGCTCCGT |
| | CGTGACCGTCCCCTCGTCGTCCCTGGGAACCCAAACCTACATTTGCAACGT |
| | CAATCACAAGCCAAGCAACACTAAGGTGGACAAGAGAGTGGAGCCCAAGT |
| | CCTGCGATAAGACCCACACCTGTCCTCCCTGTCCGGCACCTGAACTGCTTG |
| | GTGGACCTTCCGTGTTCCTGTTCCCGCCCAAGCCAAAAGACACCCTGTATA |
| | TCACTCGCGAACCGGAAGTCACTTGCGTGGTCGTGGACGTGTCCCACGAGG |
| | ACCCCGAGGTCAAGTTTAATTGGTACGTGGACGGAGTGGAAGTGCACAAC |
| | GCCAAGACCAAGCCGCGGGAAGAACAGTACAACTCCACCTACCGCGTGGT |
| | GTCCGTCCTGACTGTGCTCCACCAGGACTGGCTGAACGGAAAGGAGTACA |
| | AGTGCAAAGTGTCCAACAAGGCACTGCCAGCCCCTATCGAAAAGACTATC |
| | TCCAAGGCCAAGGGCCAACCTAGGGAGCCCCAGGTGTACACGTTGCCTCCT |
| | TCCCGCGAAGAAATGACTAAGAACCAGGTGTCGCTGACCTGTCTCGTGAAA |
| | GGGTTCTACCCCTCTGACATCGCCGTGGAATGGGAGTCAAACGGACAGCCT |
| | GAGAACAACTATAAGACCACACCACCTGTCCTGGACTCCGACGGCTCCTTC |
| | TTCCTGTACTCAAAGTTGACCGTGGACAAGTCGCGGTGGCAACAGGGCAAC |
| | GTGTTCTCTTGCTCCGTGATGCACGAAGCCCTGCACAACCACTACACCCAA |
| | AAGTCGCTCAGCCTCTCCCCCGGAAAG (SEQ ID NO: 2622) |
| LCDR1 | RASQDISNYLA (SEQ ID NO: 2599) |
| (Combined) | |
| LCDR2 | RASSLQS (SEQ ID NO: 2600) |
| (Combined) | |
| LCDR3 | FQYRHMPSQT (SEQ ID NO: 2601) |
| (Combined) | |
| LCDR1 | RASQDISNYLA (SEQ ID NO: 2599) |
| (Kabat) | |
| LCDR2 | RASSLQS (SEQ ID NO: 2600) |
| (Kabat) | |
| LCDR3 | FQYRHMPSQT (SEQ ID NO: 2601) |
| (Kabat) | |
| LCDR1 | SQDISNY (SEQ ID NO: 2602) |
| (Chothia) | |
| LCDR2 | RAS (SEQ ID NO: 2603) |
| (Chothia) | |
| LCDR3 | YRHMPSQ (SEQ ID NO: 2604) |
| (Chothia) | |
| LCDR1 | QDISNY (SEQ ID NO: 2605) |
| (IMGT) | |
| LCDR2 | RAS (SEQ ID NO: 2603) |
| (IMGT) | |
| LCDR3 | FQYRHMPSQT (SEQ ID NO: 2601) |
| (IMGT) | |
| VL | DIQMTQSPSSLSASVGDRVTITCRASQDISNYLAWYQQKPGKAPKLLIYRASSL |
| | QSGVPSRFSGSGSGTDFTLTISSLQPEDFATYYCFQYRHMPSQTFGQGTKVEIK |
| | (SEQ ID NO: 2606) |
| DNA VL | GACATTCAGATGACCCAGTCCCCGTCGTCCCTGTCCGCATCCGTGGGCGAC |
| | AGAGTCACCATCACTTGCCGGGCCTCACAGGATATTTCCAACTACCTGGCC |
| | TGGTATCAGCAGAAGCCTGGAAAGGCCCCGAAGCTGCTGATCTACCGGGC |
| | GTCCTCCTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCTTCCAGTACCGGCACATGCCCTCACAAACCTTCGGACAGGG |
| | CACCAAAGTCGAGATCAAG (SEQ ID NO: 2613) |
| Light Chain | DIQMTQSPSSLSASVGDRVTITCRASQDISNYLAWYQQKPGKAPKLLIYRASSL |
| | QSGVPSRFSGSGSGTDFTLTISSLQPEDFATYYCFQYRHMPSQTFGQGTKVEIK |
| | RTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGE |
| | C (SEQ ID NO: 2608) |
| DNA Light | GACATTCAGATGACCCAGTCCCCGTCGTCCCTGTCCGCATCCGTGGGCGAC |
| Chain | AGAGTCACCATCACTTGCCGGGCCTCACAGGATATTTCCAACTACCTGGCC |
| | TGGTATCAGCAGAAGCCTGGAAAGGCCCCGAAGCTGCTGATCTACCGGGC |
| | GTCCTCCTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCTTCCAGTACCGGCACATGCCCTCACAAACCTTCGGACAGGG |
| | CACCAAAGTCGAGATCAAGCGTACGGTGGCCGCTCCCAGCGTGTTCATCTT |
| | CCCCCCCAGCGACGAGCAGCTGAAGAGCGGCACCGCCAGCGTGGTGTGCC |
| | TGCTGAACAACTTCTACCCCCGGGAGGCCAAGGTGCAGTGGAAGGTGGAC |
| | AACGCCCTGCAGAGCGGCAACAGCCAGGAGAGCGTCACCGAGCAGGACA |
| | GCAAGGACTCCACCTACAGCCTGAGCAGCACCCTGACCCTGAGCAAGGCC |
| | GACTACGAGAAGCATAAGGTGTACGCCTGCGAGGTGACCCACCAGGGCCT |
| | GTCCAGCCCCGTGACCAAGAGCTTCAACAGGGGCGAGTGC (SEQ ID NO: 2614) |

MOR44746A

| | |
|---|---|
| HCDR1 | GDSVSSSSAAWN (SEQ ID NO: 2623) |
| (Combined) | |
| HCDR2 | HIGYRSKWYNEYAVSVKS (SEQ ID NO: 2624) |
| (Combined) | |
| HCDR3 | GMYGSVPYKEGYYFDI (SEQ ID NO: 2625) |
| (Combined) | |
| HCDR1 | SSSAAWN (SEQ ID NO: 2626) |
| (Kabat) | |
| HCDR2 | HIGYRSKWYNEYAVSVKS (SEQ ID NO: 2624) |
| (Kabat) | |
| HCDR3 | GMYGSVPYKEGYYFDI (SEQ ID NO: 2625) |
| (Kabat) | |
| HCDR1 | GDSVSSSSA (SEQ ID NO: 2627) |
| (Chothia) | |
| HCDR2 | GYRSKWY (SEQ ID NO: 2628) |
| (Chothia) | |
| HCDR3 | GMYGSVPYKEGYYFDI (SEQ ID NO: 2625) |
| (Chothia) | |
| HCDR1 | GDSVSSSSAA (SEQ ID NO: 2629) |
| (IMGT) | |
| HCDR2 | IGYRSKWYN (SEQ ID NO: 2630) |
| (IMGT) | |
| HCDR3 | ARGMYGSVPYKEGYYFDI (SEQ ID NO: 2631) |
| (IMGT) | |
| VH | QVQLQQSGPGLVKPSQTLSLTCAISGDSVSSSSAAWNWIRQSPSRGLEWLGHI |
| | GYRSKWYNEYAVSVKSRITINPDTSKNQFSLQLNSVTPEDTAVYYCARGMYG |
| | SVPYKEGYYFDIWGQGTLVTVSS (SEQ ID NO: 2632) |
| DNA VH | CAGGTGCAATTGCAGCAGAGCGGTCCGGGCCTGGTGAAACCGAGCCAGAC |
| | CCTGAGCCTGACCTGCGCGATTTCCGGAGATAGCGTGAGCTCTTCTTCTGCT |
| | GCTTGGAACTGGATTCGTCAGAGCCCGAGCCGTGGCCTCGAGTGGCTGGGC |
| | CATATCGGTTACCGTAGCAAATGGTACAACGAATATGCCGTGAGCGTGAA |
| | AAGCCGCATTACCATTAACCCGGATACTTCGAAAAACCAGTTTAGCCTGCA |
| | ACTGAACAGCGTGACCCCGGAAGATACGGCCGTGTATTATTGCGCGCGTGG |
| | TATGTACGGTTCTGTTCCCTACAAAGAAGGTTACTACTTCGATATTTGGGGC |
| | CAAGGCACCCTGGTGACTGTTAGCTCA (SEQ ID NO: 2633) |
| Heavy Chain | QVQLQQSGPGLVKPSQTLSLTCAISGDSVSSSSAAWNWIRQSPSRGLEWLGHI |
| | GYRSKWYNEYAVSVKSRITINPDTSKNQFSLQLNSVTPEDTAVYYCARGMYG |
| | SVPYKEGYYFDIWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYICN |
| | VNHKPSNTKVDKRVEPKSCDKTHTCPPCPAPEAAGGPSVFLFPPKPKDTLMISR |
| | TPEVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKLTV |
| | DKSRWQQGNVFSCSVMHEALHNHYTQKSLSLSPGK (SEQ ID NO: 2634) |
| DNA Heavy | CAGGTGCAATTGCAGCAGAGCGGTCCGGGCCTGGTGAAACCGAGCCAGAC |
| Chain | CCTGAGCCTGACCTGCGCGATTTCCGGAGATAGCGTGAGCTCTTCTTCTGCT |
| | GCTTGGAACTGGATTCGTCAGAGCCCGAGCCGTGGCCTCGAGTGGCTGGGC |
| | CATATCGGTTACCGTAGCAAATGGTACAACGAATATGCCGTGAGCGTGAA |
| | AAGCCGCATTACCATTAACCCGGATACTTCGAAAAACCAGTTTAGCCTGCA |
| | ACTGAACAGCGTGACCCCGGAAGATACGGCCGTGTATTATTGCGCGCGTGG |
| | TATGTACGGTTCTGTTCCCTACAAAGAAGGTTACTACTTCGATATTTGGGGC |
| | CAAGGCACCCTGGTGACTGTTAGCTCAGCCTCCACCAAGGGTCCATCGGTC |
| | TTCCCCCTGGCACCCTCCTCCAAGAGCACCTCTGGGGGCACAGCGGCCCTG |
| | GGCTGCCTGGTCAAGGACTACTTCCCCGAACCGGTGACGGTGTCGTGGAAC |
| | TCAGGCGCCCTGACCAGCGGCGTGCACACCTTCCCGGCTGTCCTACAGTCC |
| | TCAGGACTCTACTCCCTCAGCAGCGTGGTGACCGTGCCCTCCAGCAGCTTG |
| | GGCACCCAGACCTACATCTGCAACGTGAATCACAAGCCCAGCAACACCAA |
| | GGTGGACAAGAGAGTTGAGCCCAAATCTTGTGACAAAACTCACACATGCC |
| | CACCGTGCCCAGCACCTGAAGCAGCGGGGGGACCGTCAGTCTTCCTCTTCC |
| | CCCCAAAACCCAAGGACACCCTCATGATCTCCCGGACCCCTGAGGTCACAT |
| | GCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGA |
| | GCAGTACAACAGCACGTACCGGGTGGTCAGCGTCCTCACCGTCCTGCACCA |
| | GGACTGGCTGAATGGCAAGGAGTACAAGTGCAAGGTCTCCAACAAAGCCC |
| | TCCCAGCCCCCATCGAGAAAACCATCTCCAAAGCCAAAGGGCAGCCCCGA |
| | GAACCACAGGTGTACACCCTGCCCCCATCCCGGGAGGAGATGACCAAGAA |
| | CCAGGTCAGCCTGACCTGCCTGGTCAAAGGCTTCTATCCCAGCGACATCGC |
| | CGTGGAGTGGGAGAGCAATGGGCAGCCGGAGAACAACTACAAGACCACG |
| | CCTCCCGTGCTGGACTCCGACGGCTCCTTCTTCCTCTACAGCAAGCTCACCG |
| | TGGACAAGAGCAGGTGGCAGCAGGGGAACGTCTTCTCATGCTCCGTGATG |
| | CATGAGGCTCTGCACAACCACTACACGCAGAAGAGCCTCTCCCTGTCTCCG |
| | GGTAAA (SEQ ID NO: 2635) |
| LCDR1 | RASQGISSDLN (SEQ ID NO: 2636) |
| (Combined) | |
| LCDR2 | AASNLQS (SEQ ID NO: 2637) |
| (Combined) | |
| LCDR3 | QQYTDESMT (SEQ ID NO: 2638) |
| (Combined) | |
| LCDR1 | RASQGISSDLN (SEQ ID NO: 2636) |
| (Kabat) | |
| LCDR2 | AASNLQS (SEQ ID NO: 2637) |
| (Kabat) | |
| LCDR3 | QQYTDESMT (SEQ ID NO: 2638) |
| (Kabat) | |
| LCDR1 | SQGISSD (SEQ ID NO: 2639) |
| (Chothia) | |
| LCDR2 | AAS (SEQ ID NO: 2640) |
| (Chothia) | |
| LCDR3 | YTDESM (SEQ ID NO: 2641) |
| (Chothia) | |
| LCDR1 | QGISSD (SEQ ID NO: 2642) |
| (IMGT) | |
| LCDR2 | AAS (SEQ ID NO: 2640) |
| (IMGT) | |
| LCDR3 | QQYTDESMT (SEQ ID NO: 2638) |
| (IMGT) | |
| VL | DIQMTQSPSSLSASVGDRVTITCRASQGISSDLNWYQQKPGKAPKLLIYAASNL |
| | QSGVPSRFSGSGSGTDFTLTISSLQPEDFATYYCQQYTDESMTFGQGTKVEIK |
| | (SEQ ID NO: 2643) |
| DNA VL | GATATCCAGATGACCCAGAGCCCGAGCAGCCTGAGCGCCAGCGTGGGCGA |
| | TCGCGTGACCATTACCTGCAGAGCCAGCCAGGGTATTTCTTCTGACCTGAA |
| | CTGGTACCAGCAGAAACCGGGCAAAGCGCCGAAACTATTAATCTACGCTG |
| | CTTCTAACCTGCAAAGCGGCGTGCCGAGCCGCTTTAGCGGCAGCGGATCCG |
| | GCACCGATTTCACCCTGACCATTAGCTCTCTGCAACCGGAAGACTTTGCGA |
| | CCTATTATTGCCAGCAGTACACTGACGAATCTATGACCTTTGGCCAGGGCA |
| | CGAAAGTTGAAATTAAA (SEQ ID NO: 2644) |
| Light Chain | DIQMTQSPSSLSASVGDRVTITCRASQGISSDLNWYQQKPGKAPKLLIYAASNL |
| | QSGVPSRFSGSGSGTDFTLTISSLQPEDFATYYCQQYTDESMTFGQGTKVEIKR |
| | TVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 2645) |
| DNA Light | GATATCCAGATGACCCAGAGCCCGAGCAGCCTGAGCGCCAGCGTGGGCGA |
| Chain | TCGCGTGACCATTACCTGCAGAGCCAGCCAGGGTATTTCTTCTGACCTGAA |
| | CTGGTACCAGCAGAAACCGGGCAAAGCGCCGAAACTATTAATCTACGCTG |
| | CTTCTAACCTGCAAAGCGGCGTGCCGAGCCGCTTTAGCGGCAGCGGATCCG |
| | GCACCGATTTCACCCTGACCATTAGCTCTCTGCAACCGGAAGACTTTGCGA |
| | CCTATTATTGCCAGCAGTACACTGACGAATCTATGACCTTTGGCCAGGGCA |
| | CGAAAGTTGAAATTAAACGTACGGTGGCCGCTCCCAGCGTGTTCATCTTCC |
| | CCCCCAGCGACGAGCAGCTGAAGAGCGGCACCGCCAGCGTGGTGTGCCTG |
| | CTGAACAACTTCTACCCCCGGGAGGCCAAGGTGCAGTGGAAGGTGGACAA |
| | CGCCCTGCAGAGCGGCAACAGCCAGGAAAGCGTCACCGAGCAGGACAGCA |
| | AGGACTCCACCTACAGCCTGAGCAGCACCCTGACCCTGAGCAAGGCCGAC |
| | TACGAGAAGCACAAGGTGTACGCCTGCGAGGTGACCCACCAGGGCCTGTC |
| | CAGCCCCGTGACCAAGAGCTTCAACCGGGGCGAGTGT (SEQ ID NO: 2646) |

MOR44746B

| | |
|---|---|
| HCDR1 | GDSVSSSSAAWN (SEQ ID NO: 2623) |
| (Combined) | |
| HCDR2 | HIGYRSKWYNEYAVSVKS (SEQ ID NO: 2624) |
| (Combined) | |
| HCDR3 | GMYGSVPYKEGYYFDI (SEQ ID NO: 2625) |
| (Combined) | |
| HCDR1 | SSSAAWN (SEQ ID NO: 2626) |
| (Kabat) | |
| HCDR2 | HIGYRSKWYNEYAVSVKS (SEQ ID NO: 2624) |
| (Kabat) | |
| HCDR3 | GMYGSVPYKEGYYFDI (SEQ ID NO: 2625) |
| (Kabat) | |
| HCDR1 | GDSVSSSSA (SEQ ID NO: 2627) |
| (Chothia) | |
| HCDR2 | GYRSKWY (SEQ ID NO: 2628) |
| (Chothia) | |
| HCDR3 | GMYGSVPYKEGYYFDI (SEQ ID NO: 2625) |
| (Chothia) | |
| HCDR1 | GDSVSSSSAA (SEQ ID NO: 2629) |
| (IMGT) | |
| HCDR2 | IGYRSKWYN (SEQ ID NO: 2630) |
| (IMGT) | |
| HCDR3 | ARGMYGSVPYKEGYYFDI (SEQ ID NO: 2631) |
| (IMGT) | |
| VH | QVQLQQSGPGLVKPSQTLSLTCAISGDSVSSSSAAWNWIRQSPSRGLEWLGHI |
| | GYRSKWYNEYAVSVKSRITINPDTSKNQFSLQLNSVTPEDTAVYYCARGMYG |
| | SVPYKEGYYFDIWGQGTLVTVSS (SEQ ID NO: 2632) |
| DNA VH | CAAGTGCAACTCCAGCAGTCAGGACCGGGGTTGGTCAAGCCTTCGCAGAC |
| | CCTGTCCCTCACTTGCGCCATTAGCGGAGATTCGGTGTCGTCGTCGTCAGC |
| | CGCCTGGAACTGGATTAGACAGTCCCCTTCCCGAGGGCTGGAGTGGCTGGG |
| | CCACATCGGATACCGCAGCAAGTGGTACAACGAATACGCCGTCAGCGTGA |
| | AGTCACGCATCACCATCAACCCGGATACTAGCAAGAACCAGTTCAGCCTCC |
| | AGTTGAACTCCGTGACCCCGGAGGATACCGCCGTGTACTACTGTGCGCGGG |
| | GCATGTACGGATCCGTGCCGTACAAGGAGGGATACTACTTCGACATTTGGG |
| | GCCAGGGGACTCTTGTCACCGTGTCCTCG (SEQ ID NO: 2647) |
| Heavy Chain | QVQLQQSGPGLVKPSQTLSLTCAISGDSVSSSSAAWNWIRQSPSRGLEWLGHI |
| | GYRSKWYNEYAVSVKSRITINPDTSKNQFSLQLNSVTPEDTAVYYCARGMYG |
| | SVPYKEGYYFDIWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKLTV |
| | DKSRWQQGNVFSCSVMHEALHNHYTQKSLSLSPGK (SEQ ID NO: 2648) |
| DNA Heavy | CAAGTGCAACTCCAGCAGTCAGGACCGGGGTTGGTCAAGCCTTCGCAGAC |
| Chain | CCTGTCCCTCACTTGCGCCATTAGCGGAGATTCGGTGTCGTCGTCGTCAGC |
| | CGCCTGGAACTGGATTAGACAGTCCCCTTCCCGAGGGCTGGAGTGGCTGGG |
| | CCACATCGGATACCGCAGCAAGTGGTACAACGAATACGCCGTCAGCGTGA |
| | AGTCACGCATCACCATCAACCCGGATACTAGCAAGAACCAGTTCAGCCTCC |
| | AGTTGAACTCCGTGACCCCGGAGGATACCGCCGTGTACTACTGTGCGCGGG |
| | GCATGTACGGATCCGTGCCGTACAAGGAGGGATACTACTTCGACATTTGGG |
| | GCCAGGGGACTCTTGTCACCGTGTCCTCGGCCTCCACTAAGGGCCCAAGTG |
| | TGTTTCCCCTGGCCCCCAGCAGCAAGTCTACTTCCGGCGGAACTGCTGCCC |
| | TGGGTTGCCTGGTGAAGGACTACTTCCCCGAGCCCGTGACAGTGTCCTGGA |
| | ACTCTGGGGCTCTGACTTCCGGCGTGCACACCTTCCCCGCCGTGCTGCAGA |
| | GCAGCGGCCTGTACAGCCTGAGCAGCGTGGTGACAGTGCCCTCCAGCTCTC |
| | TGGGAACCCAGACCTATATCTGCAACGTGAACCACAAGCCCAGCAACACC |
| | AAGGTGGACAAGAGAGTGGAGCCCAAGAGCTGCGACAAGACCCACACCTG |
| | CCCCCCCTGCCCAGCTCCAGAACTGCTGGGAGGGCCTTCCGTGTTCCTGTT |
| | CCCCCCCAAGCCCAAGGACACCCTGATGATCAGCAGGACCCCCGAGGTGA |
| | CCTGCGTGGTGGTGGACGTGTCCCACGAGGACCCAGAGGTGAAGTTCAACT |
| | GGTACGTGGACGGCGTGGAGGTGCACAACGCCAAGACCAAGCCCAGAGAG |
| | GAGCAGTACAACAGCACCTACAGGGTGGTGTCCGTGCTGACCGTGCTGCA |
| | CCAGGACTGGCTGAACGGCAAAGAATACAAGTGCAAAGTCTCCAACAAGG |
| | CCCTGCCAGCCCCAATCGAAAAGACAATCAGCAAGGCCAAGGGCCAGCCA |
| | CGGGAGCCCCAGGTGTACACCCTGCCCCCCAGCCGGGAGGAGATGACCAA |
| | GAACCAGGTGTCCCTGACCTGTCTGGTGAAGGGCTTCTACCCCAGCGATAT |
| | CGCCGTGGAGTGGGAGAGCAACGGCCAGCCCGAGAACAACTACAAGACCA |
| | CCCCCCCAGTGCTGGACAGCGACGGCAGCTTCTTCCTGTACAGCAAGCTGA |
| | CCGTGGACAAGTCCAGGTGGCAGCAGGGCAACGTGTTCAGCTGCAGCGTG |
| | ATGCACGAGGCCCTGCACAACCACTACACCCAGAAGTCCCTGAGCCTGAG |
| | CCCCGGCAAG (SEQ ID NO: 2649) |
| LCDR1 | RASQGISSDLN (SEQ ID NO: 2636) |
| (Combined) | |
| LCDR2 | AASNLQS (SEQ ID NO: 2637) |
| (Combined) | |
| LCDR3 | QQYTDESMT (SEQ ID NO: 2638) |
| (Combined) | |
| LCDR1 | RASQGISSDLN (SEQ ID NO: 2636) |
| (Kabat) | |
| LCDR2 | AASNLQS (SEQ ID NO: 2637) |
| (Kabat) | |
| LCDR3 | QQYTDESMT (SEQ ID NO: 2638) |
| (Kabat) | |
| LCDR1 | SQGISSD (SEQ ID NO: 2639) |
| (Chothia) | |
| LCDR2 | AAS (SEQ ID NO: 2640) |
| (Chothia) | |
| LCDR3 | YTDESM (SEQ ID NO: 2641) |
| (Chothia) | |
| LCDR1 | QGISSD (SEQ ID NO: 2642) |
| (IMGT) | |
| LCDR2 | AAS (SEQ ID NO: 2640) |
| (IMGT) | |
| LCDR3 | QQYTDESMT (SEQ ID NO: 2638) |
| (IMGT) | |
| VL | DIQMTQSPSSLSASVGDRVTITCRASQGISSDLNWYQQKPGKAPKLLIYAASNL |
| | QSGVPSRFSGSGSGTDFTLTISSLQPEDFATYYCQQYTDESMTFGQGTKVEIK |
| | (SEQ ID NO: 2643) |
| DNA VL | GACATTCAGATGACCCAGTCCCCGTCGTCCCTGTCCGCATCCGTGGGCGAC |
| | AGAGTCACCATCACTTGCCGGGCCTCACAGGGAATTTCCTCCGACCTGAAC |
| | TGGTATCAGCAGAAGCCTGGAAAGGCCCCGAAGCTGCTGATCTACGCCGC |
| | GTCCAACTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCCAACAGTACACAGATGAATCCATGACCTTCGGACAGGGCAC |
| | CAAAGTCGAGATCAAG (SEQ ID NO: 2650) |
| Light Chain | DIQMTQSPSSLSASVGDRVTITCRASQGISSDLNWYQQKPGKAPKLLIYAASNL |
| | QSGVPSRFSGSGSGTDFTLTISSLQPEDFATYYCQQYTDESMTFGQGTKVEIKR |
| | TVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 2645) |
| DNA Light | GACATTCAGATGACCCAGTCCCCGTCGTCCCTGTCCGCATCCGTGGGCGAC |
| Chain | AGAGTCACCATCACTTGCCGGGCCTCACAGGGAATTTCCTCCGACCTGAAC |
| | TGGTATCAGCAGAAGCCTGGAAAGGCCCCGAAGCTGCTGATCTACGCCGC |
| | GTCCAACTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCCAACAGTACACAGATGAATCCATGACCTTCGGACAGGGCAC |
| | CAAAGTCGAGATCAAGCGTACGGTGGCCGCTCCCAGCGTGTTCATCTTCCC |
| | CCCCAGCGACGAGCAGCTGAAGAGCGGCACCGCCAGCGTGGTGTGCCTGC |
| | TGAACAACTTCTACCCCCGGGAGGCCAAGGTGCAGTGGAAGGTGGACAAC |
| | GCCCTGCAGAGCGGCAACAGCCAGGAGAGCGTCACCGAGCAGGACAGCA |
| | AGGACTCCACCTACAGCCTGAGCAGCACCCTGACCCTGAGCAAGGCCGAC |
| | TACGAGAAGCATAAGGTGTACGCCTGCGAGGTGACCCACCAGGGCCTGTC |
| | CAGCCCCGTGACCAAGAGCTTCAACAGGGGCGAGTGC (SEQ ID NO: 2651) |

MOR44746C

| | |
|---|---|
| HCDR1 | GDSVSSSSAAWN (SEQ ID NO: 2623) |
| (Combined) | |
| HCDR2 | HIGYRSKWYNEYAVSVKS (SEQ ID NO: 2624) |
| (Combined) | |
| HCDR3 | GMYGSVPYKEGYYFDI (SEQ ID NO: 2625) |
| (Combined) | |
| HCDR1 | SSSAAWN (SEQ ID NO: 2626) |
| (Kabat) | |
| HCDR2 | HIGYRSKWYNEYAVSVKS (SEQ ID NO: 2624) |
| (Kabat) | |
| HCDR3 | GMYGSVPYKEGYYFDI (SEQ ID NO: 2625) |
| (Kabat) | |
| HCDR1 | GDSVSSSSA (SEQ ID NO: 2627) |
| (Chothia) | |
| HCDR2 | GYRSKWY (SEQ ID NO: 2628) |
| (Chothia) | |
| HCDR3 | GMYGSVPYKEGYYFDI (SEQ ID NO: 2625) |
| (Chothia) | |
| HCDR1 | GDSVSSSSAA (SEQ ID NO: 2629) |
| (IMGT) | |
| HCDR2 | IGYRSKWYN (SEQ ID NO: 2630) |
| (IMGT) | |
| HCDR3 | ARGMYGSVPYKEGYYFDI (SEQ ID NO: 2631) |
| (IMGT) | |
| VH | QVQLQQSGPGLVKPSQTLSLTCAISGDSVSSSSAAWNWIRQSPSRGLEWLGHI |
| | GYRSKWYNEYAVSVKSRITINPDTSKNQFSLQLNSVTPEDTAVYYCARGMYG |
| | SVPYKEGYYFDIWGQGTLVTVSS (SEQ ID NO: 2632) |
| DNA VH | CAAGTGCAACTCCAGCAGTCAGGACCGGGGTTGGTCAAGCCTTCGCAGAC |
| | CCTGTCCCTCACTTGCGCCATTAGCGGAGATTCGGTGTCGTCGTCGTCAGC |
| | CGCCTGGAACTGGATTAGACAGTCCCCTTCCCGAGGGCTGGAGTGGCTGGG |
| | CCACATCGGATACCGCAGCAAGTGGTACAACGAATACGCCGTCAGCGTGA |
| | AGTCACGCATCACCATCAACCCGGATACTAGCAAGAACCAGTTCAGCCTCC |
| | AGTTGAACTCCGTGACCCCGGAGGATACCGCCGTGTACTACTGTGCGCGGG |
| | GCATGTACGGATCCGTGCCGTACAAGGAGGGATACTACTTCGACATTTGGG |
| | GCCAGGGGACTCTTGTCACCGTGTCCTCG (SEQ ID NO: 2647) |
| Heavy Chain | QVQLQQSGPGLVKPSQTLSLTCAISGDSVSSSSAAWNWIRQSPSRGLEWLGHI |
| | GYRSKWYNEYAVSVKSRITINPDTSKNQFSLQLNSVTPEDTAVYYCARGMYG |
| | SVPYKEGYYFDIWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLMISR |
| | TPEVTCVVVAVSHEDPEVKFNWYVDGVEVHNAKTKPREEQYNSTYRVVSVL |
| | TVLHQDWLNGKEYKCKVSNKALAAPIEKTISKAKGQPREPQVYTLPPSREEMT |
| | KNQVSLTCLVKGFYPSDIAVEWESNGQPENNYKTTPPVLDSDGSFFLYSKLTV |
| | DKSRWQQGNVFSCSVMHEALHNHYTQKSLSLSPGK (SEQ ID NO: 2652) |
| DNA Heavy | CAAGTGCAACTCCAGCAGTCAGGACCGGGGTTGGTCAAGCCTTCGCAGAC |
| Chain | CCTGTCCCTCACTTGCGCCATTAGCGGAGATTCGGTGTCGTCGTCGTCAGC |
| | CGCCTGGAACTGGATTAGACAGTCCCCTTCCCGAGGGCTGGAGTGGCTGGG |
| | CCACATCGGATACCGCAGCAAGTGGTACAACGAATACGCCGTCAGCGTGA |
| | AGTCACGCATCACCATCAACCCGGATACTAGCAAGAACCAGTTCAGCCTCC |
| | AGTTGAACTCCGTGACCCCGGAGGATACCGCCGTGTACTACTGTGCGCGGG |
| | GCATGTACGGATCCGTGCCGTACAAGGAGGGATACTACTTCGACATTTGGG |
| | GCCAGGGGACTCTTGTCACCGTGTCCTCGGCCTCCACTAAGGGCCCGTCAG |
| | TGTTCCCCCTTGCGCCATCCTCGAAGTCAACCTCCGGAGGAACTGCCGCAC |
| | TGGGTTGCCTCGTGAAAGACTATTTCCCGGAACCCGTCACTGTCTCCTGGA |
| | ACTCAGGAGCGCTCACCAGCGGAGTGCATACCTTTCCTGCGGTGCTGCAGT |
| | CCAGCGGCCTGTACTCCCTGAGCTCCGTCGTGACCGTCCCCTCGTCGTCCCT |
| | GGGAACCCAAACCTACATTTGCAACGTCAATCACAAGCCAAGCAACACTA |
| | AGGTGGACAAGAGAGTGGAGCCCAAGTCCTGCGATAAGACCCACACCTGT |
| | CCTCCCTGTCCGGCACCTGAACTGCTTGGTGGACCTTCCGTGTTCCTGTTCC |
| | CGCCCAAGCCAAAAGACACCCTGATGATCTCCCGCACTCCGGAAGTCACTT |
| | GCGTGGTCGTGGCCGTGTCCCACGAGGACCCCGAGGTCAAGTTTAATTGGT |
| | ACGTGGACGGAGTGGAAGTGCACAACGCCAAGACCAAGCCGCGGGAAGA |
| | ACAGTACAACTCCACCTACCGCGTGGTGTCCGTCCTGACTGTGCTCCACCA |
| | GGACTGGCTGAACGGAAAGGAGTACAAGTGCAAAGTGTCCAACAAGGCAC |
| | TGGCTGCCCCTATCGAAAAGACTATCTCCAAGGCCAAGGGCCAACCTAGG |
| | GAGCCCCAGGTGTACACGTTGCCTCCTTCCCGCGAAGAAATGACTAAGAAC |
| | CAGGTGTCGCTGACCTGTCTCGTGAAAGGGTTCTACCCCTCTGACATCGCC |
| | GTGGAATGGGAGTCAAACGGACAGCCTGAGAACAACTATAAGACCACACC |
| | ACCTGTCCTGGACTCCGACGGCTCCTTCTTCCTGTACTCAAAGTTGACCGTG |
| | GACAAGTCGCGGTGGCAACAGGGCAACGTGTTCTCTTGCTCCGTGATGCAC |
| | GAAGCCCTGCACAACCACTACACCCAAAAGTCGCTCAGCCTCTCCCCCGGA |
| | AAG (SEQ ID NO: 2653) |
| LCDR1 | RASQGISSDLN (SEQ ID NO: 2636) |
| (Combined) | |
| LCDR2 | AASNLQS (SEQ ID NO: 2637) |
| (Combined) | |
| LCDR3 | QQYTDESMT (SEQ ID NO: 2638) |
| (Combined) | |
| LCDR1 | RASQGISSDLN (SEQ ID NO: 2636) |
| (Kabat) | |
| LCDR2 | AASNLQS (SEQ ID NO: 2637) |
| (Kabat) | |
| LCDR3 | QQYTDESMT (SEQ ID NO: 2638) |
| (Kabat) | |
| LCDR1 | SQGISSD (SEQ ID NO: 2639) |
| (Chothia) | |
| LCDR2 | AAS (SEQ ID NO: 2640) |
| (Chothia) | |
| LCDR3 | YTDESM (SEQ ID NO: 2641) |
| (Chothia) | |
| LCDR1 | (SEQ ID NO: 2642) |
| (IMGT) | |
| LCDR2 | AAS (SEQ ID NO: 2640) |
| (IMGT) | |
| LCDR3 | QQYTDESMT (SEQ ID NO: 2638) |
| (IMGT) | |
| VL | DIQMTQSPSSLSASVGDRVTITCRASQGISSDLNWYQQKPGKAPKLLIYAASNL |
| | QSGVPSRFSGSGSGTDFTLTISSLQPEDFATYYCQQYTDESMTFGQGTKVEIK |
| | (SEQ ID NO: 2643) |
| DNA VL | GACATTCAGATGACCCAGTCCCCGTCGTCCCTGTCCGCATCCGTGGGCGAC |
| | AGAGTCACCATCACTTGCCGGGCCTCACAGGGAATTTCCTCCGACCTGAAC |
| | TGGTATCAGCAGAAGCCTGGAAAGGCCCCGAAGCTGCTGATCTACGCCGC |
| | GTCCAACTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCCAACAGTACACAGATGAATCCATGACCTTCGGACAGGGCAC |
| | CAAAGTCGAGATCAAG (SEQ ID NO: 2650) |
| Light Chain | DIQMTQSPSSLSASVGDRVTITCRASQGISSDLNWYQQKPGKAPKLLIYAASNL |
| | QSGVPSRFSGSGSGTDFTLTISSLQPEDFATYYCQQYTDESMTFGQGTKVEIKR |
| | TVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 2645) |
| DNA Light | GACATTCAGATGACCCAGTCCCCGTCGTCCCTGTCCGCATCCGTGGGCGAC |
| Chain | AGAGTCACCATCACTTGCCGGGCCTCACAGGGAATTTCCTCCGACCTGAAC |
| | TGGTATCAGCAGAAGCCTGGAAAGGCCCCGAAGCTGCTGATCTACGCCGC |
| | GTCCAACTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCCAACAGTACACAGATGAATCCATGACCTTCGGACAGGGCAC |
| | CAAAGTCGAGATCAAGCGTACGGTGGCCGCTCCCAGCGTGTTCATCTTCCC |
| | CCCCAGCGACGAGCAGCTGAAGAGCGGCACCGCCAGCGTGGTGTGCCTGC |
| | TGAACAACTTCTACCCCCGGGAGGCCAAGGTGCAGTGGAAGGTGGACAAC |
| | GCCCTGCAGAGCGGCAACAGCCAGGAGAGCGTCACCGAGCAGGACAGCA |
| | AGGACTCCACCTACAGCCTGAGCAGCACCCTGACCCTGAGCAAGGCCGAC |
| | TACGAGAAGCATAAGGTGTACGCCTGCGAGGTGACCCACCAGGGCCTGTC |
| | CAGCCCCGTGACCAAGAGCTTCAACAGGGGCGAGTGC (SEQ ID NO: 2651) |

MOR44746D

| | |
|---|---|
| HCDR1 | GDSVSSSSAAWN (SEQ ID NO: 2623) |
| (Combined) | |
| HCDR2 | HIGYRSKWYNEYAVSVKS (SEQ ID NO: 2624) |
| (Combined) | |
| HCDR3 | GMYGSVPYKEGYYFDI (SEQ ID NO: 2625) |
| (Combined) | |
| HCDR1 | SSSAAWN (SEQ ID NO: 2626) |
| (Kabat) | |
| HCDR2 | HIGYRSKWYNEYAVSVKS (SEQ ID NO: 2624) |
| (Kabat) | |
| HCDR3 | GMYGSVPYKEGYYFDI (SEQ ID NO: 2625) |
| (Kabat) | |
| HCDR1 | GDSVSSSSA (SEQ ID NO: 2627) |
| (Chothia) | |
| HCDR2 | GYRSKWY (SEQ ID NO: 2628) |
| (Chothia) | |
| HCDR3 | GMYGSVPYKEGYYFDI (SEQ ID NO: 2625) |
| (Chothia) | |
| HCDR1 | GDSVSSSSAA (SEQ ID NO: 2629) |
| (IMGT) | |
| HCDR2 | IGYRSKWYN (SEQ ID NO: 2630) |
| (IMGT) | |
| HCDR3 | ARGMYGSVPYKEGYYFDI (SEQ ID NO: 2631) |
| (IMGT) | |
| VH | QVQLQQSGPGLVKPSQTLSLTCAISGDSVSSSSAAWNWIRQSPSRGLEWLGHI |
| | GYRSKWYNEYAVSVKSRITINPDTSKNQFSLQLNSVTPEDTAVYYCARGMYG |
| | SVPYKEGYYFDIWGQGTLVTVSS (SEQ ID NO: 2632) |
| DNA VH | CAAGTGCAACTCCAGCAGTCAGGACCGGGGTTGGTCAAGCCTTCGCAGAC |
| | CCTGTCCCTCACTTGCGCCATTAGCGGAGATTCGGTGTCGTCGTCGTCAGC |
| | CGCCTGGAACTGGATTAGACAGTCCCCTTCCCGAGGGCTGGAGTGGCTGGG |
| | CCACATCGGATACCGCAGCAAGTGGTACAACGAATACGCCGTCAGCGTGA |
| | AGTCACGCATCACCATCAACCCGGATACTAGCAAGAACCAGTTCAGCCTCC |
| | AGTTGAACTCCGTGACCCCGGAGGATACCGCCGTGTACTACTGTGCGCGGG |
| | GCATGTACGGATCCGTGCCGTACAAGGAGGGATACTACTTCGACATTTGGG |
| | GCCAGGGGACTCTTGTCACCGTGTCCTCG (SEQ ID NO: 2647) |
| Heavy Chain | QVQLQQSGPGLVKPSQTLSLTCAISGDSVSSSSAAWNWIRQSPSRGLEWLGHI |
| | GYRSKWYNEYAVSVKSRITINPDTSKNQFSLQLNSVTPEDTAVYYCARGMYG |
| | SVPYKEGYYFDIWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKLTV |
| | DKSRWQQGNVFSCSVLHEALHSHYTQKSLSLSPGK (SEQ ID NO: 2654) |
| DNA Heavy | CAAGTGCAACTCCAGCAGTCAGGACCGGGGTTGGTCAAGCCTTCGCAGAC |
| Chain | CCTGTCCCTCACTTGCGCCATTAGCGGAGATTCGGTGTCGTCGTCGTCAGC |
| | CGCCTGGAACTGGATTAGACAGTCCCCTTCCCGAGGGCTGGAGTGGCTGGG |
| | CCACATCGGATACCGCAGCAAGTGGTACAACGAATACGCCGTCAGCGTGA |
| | AGTCACGCATCACCATCAACCCGGATACTAGCAAGAACCAGTTCAGCCTCC |
| | AGTTGAACTCCGTGACCCCGGAGGATACCGCCGTGTACTACTGTGCGCGGG |
| | GCATGTACGGATCCGTGCCGTACAAGGAGGGATACTACTTCGACATTTGGG |
| | GCCAGGGGACTCTTGTCACCGTGTCCTCGGCCTCCACTAAGGGCCCGTCAG |
| | TGTTCCCCCTTGCGCCATCCTCGAAGTCAACCTCCGGAGGAACTGCCGCAC |
| | TGGGTTGCCTCGTGAAAGACTATTTCCCGGAACCCGTCACTGTCTCCTGGA |
| | ACTCAGGAGCGCTCACCAGCGGAGTGCATACCTTTCCTGCGGTGCTGCAGT |
| | CCAGCGGCCTGTACTCCCTGAGCTCCGTCGTGACCGTCCCCTCGTCGTCCCT |
| | GGGAACCCAAACCTACATTTGCAACGTCAATCACAAGCCAAGCAACACTA |
| | AGGTGGACAAGAGAGTGGAGCCCAAGTCCTGCGATAAGACCCACACCTGT |
| | CCTCCCTGTCCGGCACCTGAACTGCTTGGTGGACCTTCCGTGTTCCTGTTCC |
| | CGCCCAAGCCAAAAGACACCCTGATGATCTCCCGCACTCCGGAAGTCACTT |
| | GCGTGGTCGTGGACGTGTCCCACGAGGACCCCGAGGTCAAGTTTAATTGGT |
| | ACGTGGACGGAGTGGAAGTGCACAACGCCAAGACCAAGCCGCGGGAAGA |
| | ACAGTACAACTCCACCTACCGCGTGGTGTCCGTCCTGACTGTGCTCCACCA |
| | GGACTGGCTGAACGGAAAGGAGTACAAGTGCAAAGTGTCCAACAAGGCAC |
| | TGCCAGCCCCTATCGAAAAGACTATCTCCAAGGCCAAGGGCCAACCTAGG |
| | GAGCCCCAGGTGTACACGTTGCCTCCTTCCCGCGAAGAAATGACTAAGAAC |
| | CAGGTGTCGCTGACCTGTCTCGTGAAAGGGTTCTACCCCTCTGACATCGCC |
| | GTGGAATGGGAGTCAAACGGACAGCCTGAGAACAACTATAAGACCACACC |
| | ACCTGTCCTGGACTCCGACGGCTCCTTCTTCCTGTACTCAAAGTTGACCGTG |
| | GACAAGTCGCGGTGGCAACAGGGCAACGTGTTCTCTTGCTCCGTGCTGCAC |
| | GAAGCCCTGCACAGCCACTACACCCAAAAGTCGCTCAGCCTCTCCCCCGGA |
| | AAG (SEQ ID NO: 2655) |
| LCDR1 | RASQGISSDLN (SEQ ID NO: 2636) |
| (Combined) | |
| LCDR2 | AASNLQS (SEQ ID NO: 2637) |
| (Combined) | |
| LCDR3 | QQYTDESMT (SEQ ID NO: 2638) |
| (Combined) | |
| LCDR1 | RASQGISSDLN (SEQ ID NO: 2636) |
| (Kabat) | |
| LCDR2 | AASNLQS (SEQ ID NO: 2637) |
| (Kabat) | |
| LCDR3 | QQYTDESMT (SEQ ID NO: 2638) |
| (Kabat) | |
| LCDR1 | SQGISSD (SEQ ID NO: 2639) |
| (Chothia) | |
| LCDR2 | AAS (SEQ ID NO: 2640) |
| (Chothia) | |
| LCDR3 | YTDESM (SEQ ID NO: 2641) |
| (Chothia) | |
| LCDR1 | QGISSD (SEQ ID NO: 2642) |
| (IMGT) | |
| LCDR2 | AAS (SEQ ID NO: 2640) |
| (IMGT) | |
| LCDR3 | QQYTDESMT (SEQ ID NO: 2638) |
| (IMGT) | |
| VL | DIQMTQSPSSLSASVGDRVTITCRASQGISSDLNWYQQKPGKAPKLLIYAASNL |
| | QSGVPSRFSGSGSGTDFTLTISSLQPEDFATYYCQQYTDESMTFGQGTKVEIK |
| | (SEQ ID NO: 2643) |
| DNA VL | GACATTCAGATGACCCAGTCCCCGTCGTCCCTGTCCGCATCCGTGGGCGAC |
| | AGAGTCACCATCACTTGCCGGGCCTCACAGGGAATTTCCTCCGACCTGAAC |
| | TGGTATCAGCAGAAGCCTGGAAAGGCCCCGAAGCTGCTGATCTACGCCGC |
| | GTCCAACTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCCAACAGTACACAGATGAATCCATGACCTTCGGACAGGGCAC |
| | CAAAGTCGAGATCAAG (SEQ ID NO: 2650) |
| Light Chain | DIQMTQSPSSLSASVGDRVTITCRASQGISSDLNWYQQKPGKAPKLLIYAASNL |
| | QSGVPSRFSGSGSGTDFTLTISSLQPEDFATYYCQQYTDESMTFGQGTKVEIKR |
| | TVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 2645) |
| DNA Light | GACATTCAGATGACCCAGTCCCCGTCGTCCCTGTCCGCATCCGTGGGCGAC |
| Chain | AGAGTCACCATCACTTGCCGGGCCTCACAGGGAATTTCCTCCGACCTGAAC |
| | TGGTATCAGCAGAAGCCTGGAAAGGCCCCGAAGCTGCTGATCTACGCCGC |
| | GTCCAACTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCCAACAGTACACAGATGAATCCATGACCTTCGGACAGGGCAC |
| | CAAAGTCGAGATCAAGCGTACGGTGGCCGCTCCCAGCGTGTTCATCTTCCC |
| | CCCCAGCGACGAGCAGCTGAAGAGCGGCACCGCCAGCGTGGTGTGCCTGC |
| | TGAACAACTTCTACCCCCGGGAGGCCAAGGTGCAGTGGAAGGTGGACAAC |
| | GCCCTGCAGAGCGGCAACAGCCAGGAGAGCGTCACCGAGCAGGACAGCA |
| | AGGACTCCACCTACAGCCTGAGCAGCACCCTGACCCTGAGCAAGGCCGAC |
| | TACGAGAAGCATAAGGTGTACGCCTGCGAGGTGACCCACCAGGGCCTGTC |
| | CAGCCCCGTGACCAAGAGCTTCAACAGGGGCGAGTGC (SEQ ID NO: 2651) |

MOR44746E

| | |
|---|---|
| HCDR1 | GDSVSSSSAAWN (SEQ ID NO: 2623) |
| (Combined) | |
| HCDR2 | HIGYRSKWYNEYAVSVKS (SEQ ID NO: 2624) |
| (Combined) | |
| HCDR3 | GMYGSVPYKEGYYFDI (SEQ ID NO: 2625) |
| (Combined) | |
| HCDR1 | SSSAAWN (SEQ ID NO: 2626) |
| (Kabat) | |
| HCDR2 | HIGYRSKWYNEYAVSVKS (SEQ ID NO: 2624) |
| (Kabat) | |
| HCDR3 | GMYGSVPYKEGYYFDI (SEQ ID NO: 2625) |
| (Kabat) | |
| HCDR1 | GDSVSSSSA (SEQ ID NO: 2627) |
| (Chothia) | |
| HCDR2 | GYRSKWY (SEQ ID NO: 2628) |
| (Chothia) | |
| HCDR3 | GMYGSVPYKEGYYFDI (SEQ ID NO: 2625) |
| (Chothia) | |
| HCDR1 | GDSVSSSSAA (SEQ ID NO: 2629) |
| (IMGT) | |
| HCDR2 | IGYRSKWYN (SEQ ID NO: 2630) |
| (IMGT) | |
| HCDR3 | ARGMYGSVPYKEGYYFDI (SEQ ID NO: 2631) |
| (IMGT) | |
| VH | QVQLQQSGPGLVKPSQTLSLTCAISGDSVSSSSAAWNWIRQSPSRGLEWLGHI |
| | GYRSKWYNEYAVSVKSRITINPDTSKNQFSLQLNSVTPEDTAVYYCARGMYG |
| | SVPYKEGYYFDIWGQGTLVTVSS (SEQ ID NO: 2632) |
| DNA VH | CAAGTGCAACTCCAGCAGTCAGGACCGGGGTTGGTCAAGCCTTCGCAGAC |
| | CCTGTCCCTCACTTGCGCCATTAGCGGAGATTCGGTGTCGTCGTCGTCAGC |
| | CGCCTGGAACTGGATTAGACAGTCCCCTTCCCGAGGGCTGGAGTGGCTGGG |
| | CCACATCGGATACCGCAGCAAGTGGTACAACGAATACGCCGTCAGCGTGA |
| | AGTCACGCATCACCATCAACCCGGATACTAGCAAGAACCAGTTCAGCCTCC |
| | AGTTGAACTCCGTGACCCCGGAGGATACCGCCGTGTACTACTGTGCGCGGG |
| | GCATGTACGGATCCGTGCCGTACAAGGAGGGATACTACTTCGACATTTGGG |
| | GCCAGGGGACTCTTGTCACCGTGTCCTCG (SEQ ID NO: 2647) |
| Heavy Chain | QVQLQQSGPGLVKPSQTLSLTCAISGDSVSSSSAAWNWIRQSPSRGLEWLGHI |
| | GYRSKWYNEYAVSVKSRITINPDTSKNQFSLQLNSVTPEDTAVYYCARGMYG |
| | SVPYKEGYYFDIWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLMISR |
| | TPEVTCVVVAVSHEDPEVKFNWYVDGVEVHNAKTKPREEQYNSTYRVVSVL |
| | TVLHQDWLNGKEYKCKVSNKALAAPIEKTISKAKGQPREPQVYTLPPSREEMT |
| | KNQVSLTCLVKGFYPSDIAVEWESNGQPENNYKTTPPVLDSDGSFFLYSKLTV |
| | DKSRWQQGNVFSCSVLHEALHSHYTQKSLSLSPGK (SEQ ID NO: 2656) |
| DNA Heavy | CAAGTGCAACTCCAGCAGTCAGGACCGGGGTTGGTCAAGCCTTCGCAGAC |
| Chain | CCTGTCCCTCACTTGCGCCATTAGCGGAGATTCGGTGTCGTCGTCGTCAGC |
| | CGCCTGGAACTGGATTAGACAGTCCCCTTCCCGAGGGCTGGAGTGGCTGGG |
| | CCACATCGGATACCGCAGCAAGTGGTACAACGAATACGCCGTCAGCGTGA |
| | AGTCACGCATCACCATCAACCCGGATACTAGCAAGAACCAGTTCAGCCTCC |
| | AGTTGAACTCCGTGACCCCGGAGGATACCGCCGTGTACTACTGTGCGCGGG |
| | GCATGTACGGATCCGTGCCGTACAAGGAGGGATACTACTTCGACATTTGGG |
| | GCCAGGGGACTCTTGTCACCGTGTCCTCGGCCTCCACTAAGGGCCCGTCAG |
| | TGTTCCCCCTTGCGCCATCCTCGAAGTCAACCTCCGGAGGAACTGCCGCAC |
| | TGGGTTGCCTCGTGAAAGACTATTTCCCGGAACCCGTCACTGTCTCCTGGA |
| | ACTCAGGAGCGCTCACCAGCGGAGTGCATACCTTTCCTGCGGTGCTGCAGT |
| | CCAGCGGCCTGTACTCCCTGAGCTCCGTCGTGACCGTCCCCTCGTCGTCCCT |
| | GGGAACCCAAACCTACATTTGCAACGTCAATCACAAGCCAAGCAACACTA |
| | AGGTGGACAAGAGAGTGGAGCCCAAGTCCTGCGATAAGACCCACACCTGT |
| | CCTCCCTGTCCGGCACCTGAACTGCTTGGTGGACCTTCCGTGTTCCTGTTCC |
| | CGCCCAAGCCAAAAGACACCCTGATGATCTCCCGCACTCCGGAAGTCACTT |
| | GCGTGGTCGTGGCCGTGTCCCACGAGGACCCCGAGGTCAAGTTTAATTGGT |
| | ACGTGGACGGAGTGGAAGTGCACAACGCCAAGACCAAGCCGCGGGAAGA |
| | ACAGTACAACTCCACCTACCGCGTGGTGTCCGTCCTGACTGTGCTCCACCA |
| | GGACTGGCTGAACGGAAAGGAGTACAAGTGCAAAGTGTCCAACAAGGCAC |
| | TGGCTGCCCCTATCGAAAAGACTATCTCCAAGGCCAAGGGCCAACCTAGG |
| | GAGCCCCAGGTGTACACGTTGCCTCCTTCCCGCGAAGAAATGACTAAGAAC |
| | CAGGTGTCGCTGACCTGTCTCGTGAAAGGGTTCTACCCCTCTGACATCGCC |
| | GTGGAATGGGAGTCAAACGGACAGCCTGAGAACAACTATAAGACCACACC |
| | ACCTGTCCTGGACTCCGACGGCTCCTTCTTCCTGTACTCAAAGTTGACCGTG |
| | GACAAGTCGCGGTGGCAACAGGGCAACGTGTTCTCTTGCTCCGTGCTGCAC |
| | GAAGCCCTGCACAGCCACTACACCCAAAAGTCGCTCAGCCTCTCCCCCGGA |
| | AAG (SEQ ID NO: 2657) |
| LCDR1 | RASQGISSDLN (SEQ ID NO: 2636) |
| (Combined) | |
| LCDR2 | AASNLQS (SEQ ID NO: 2637) |
| (Combined) | |
| LCDR3 | QQYTDESMT (SEQ ID NO: 2638) |
| (Combined) | |
| LCDR1 | RASQGISSDLN (SEQ ID NO: 2636) |
| (Kabat) | |
| LCDR2 | AASNLQS (SEQ ID NO: 2637) |
| (Kabat) | |
| LCDR3 | QQYTDESMT (SEQ ID NO: 2638) |
| (Kabat) | |
| LCDR1 | SQGISSD (SEQ ID NO: 2639) |
| (Chothia) | |
| LCDR2 | AAS (SEQ ID NO: 2640) |
| (Chothia) | |
| LCDR3 | YTDESM (SEQ ID NO: 2641) |
| (Chothia) | |
| LCDR1 | QGISSD (SEQ ID NO: 2642) |
| (IMGT) | |
| LCDR2 | AAS (SEQ ID NO: 2640) |
| (IMGT) | |
| LCDR3 | QQYTDESMT (SEQ ID NO: 2638) |
| (IMGT) | |
| VL | DIQMTQSPSSLSASVGDRVTITCRASQGISSDLNWYQQKPGKAPKLLIYAASNL |
| | (SEQ ID NO: 2643) |
| DNA VL | GACATTCAGATGACCCAGTCCCCGTCGTCCCTGTCCGCATCCGTGGGCGAC |
| | AGAGTCACCATCACTTGCCGGGCCTCACAGGGAATTTCCTCCGACCTGAAC |
| | TGGTATCAGCAGAAGCCTGGAAAGGCCCCGAAGCTGCTGATCTACGCCGC |
| | GTCCAACTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCCAACAGTACACAGATGAATCCATGACCTTCGGACAGGGCAC |
| | CAAAGTCGAGATCAAG (SEQ ID NO: 2650) |
| Light Chain | DIQMTQSPSSLSASVGDRVTITCRASQGISSDLNWYQQKPGKAPKLLIYAASNL |
| | QSGVPSRFSGSGSGTDFTLTISSLQPEDFATYYCQQYTDESMTFGQGTKVEIKR |
| | TVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 2645) |
| DNA Light | GACATTCAGATGACCCAGTCCCCGTCGTCCCTGTCCGCATCCGTGGGCGAC |
| Chain | AGAGTCACCATCACTTGCCGGGCCTCACAGGGAATTTCCTCCGACCTGAAC |
| | TGGTATCAGCAGAAGCCTGGAAAGGCCCCGAAGCTGCTGATCTACGCCGC |
| | GTCCAACTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCCAACAGTACACAGATGAATCCATGACCTTCGGACAGGGCAC |
| | CAAAGTCGAGATCAAGCGTACGGTGGCCGCTCCCAGCGTGTTCATCTTCCC |
| | CCCCAGCGACGAGCAGCTGAAGAGCGGCACCGCCAGCGTGGTGTGCCTGC |
| | TGAACAACTTCTACCCCCGGGAGGCCAAGGTGCAGTGGAAGGTGGACAAC |
| | GCCCTGCAGAGCGGCAACAGCCAGGAGAGCGTCACCGAGCAGGACAGCA |
| | AGGACTCCACCTACAGCCTGAGCAGCACCCTGACCCTGAGCAAGGCCGAC |
| | TACGAGAAGCATAAGGTGTACGCCTGCGAGGTGACCCACCAGGGCCTGTC |
| | CAGCCCCGTGACCAAGAGCTTCAACAGGGGCGAGTGC (SEQ ID NO: 2651) |

MOR44746F

| | |
|---|---|
| HCDR1 | GDSVSSSSAAWN (SEQ ID NO: 2623) |
| (Combined) | |
| HCDR2 | HIGYRSKWYNEYAVSVKS (SEQ ID NO: 2624) |
| (Combined) | |
| HCDR3 | GMYGSVPYKEGYYFDI (SEQ ID NO: 2625) |
| (Combined) | |
| HCDR1 | SSSAAWN (SEQ ID NO: 2626) |
| (Kabat) | |
| HCDR2 | HIGYRSKWYNEYAVSVKS (SEQ ID NO: 2624) |
| (Kabat) | |
| HCDR3 | GMYGSVPYKEGYYFDI (SEQ ID NO: 2625) |
| (Kabat) | |
| HCDR1 | GDSVSSSSA (SEQ ID NO: 2627) |
| (Chothia) | |
| HCDR2 | GYRSKWY (SEQ ID NO: 2628) |
| (Chothia) | |
| HCDR3 | GMYGSVPYKEGYYFDI (SEQ ID NO: 2625) |
| (Chothia) | |
| HCDR1 | GDSVSSSSAA (SEQ ID NO: 2629) |
| (IMGT) | |
| HCDR2 | IGYRSKWYN (SEQ ID NO: 2630) |
| (IMGT) | |
| HCDR3 | ARGMYGSVPYKEGYYFDI (SEQ ID NO: 2631) |
| (IMGT) | |
| VH | QVQLQQSGPGLVKPSQTLSLTCAISGDSVSSSSAAWNWIRQSPSRGLEWLGHI |
| | GYRSKWYNEYAVSVKSRITINPDTSKNQFSLQLNSVTPEDTAVYYCARGMYG |
| | SVPYKEGYYFDIWGQGTLVTVSS (SEQ ID NO: 2632) |
| DNA VH | CAAGTGCAACTCCAGCAGTCAGGACCGGGGTTGGTCAAGCCTTCGCAGAC |
| | CCTGTCCCTCACTTGCGCCATTAGCGGAGATTCGGTGTCGTCGTCGTCAGC |
| | CGCCTGGAACTGGATTAGACAGTCCCCTTCCCGAGGGCTGGAGTGGCTGGG |
| | CCACATCGGATACCGCAGCAAGTGGTACAACGAATACGCCGTCAGCGTGA |
| | AGTCACGCATCACCATCAACCCGGATACTAGCAAGAACCAGTTCAGCCTCC |
| | AGTTGAACTCCGTGACCCCGGAGGATACCGCCGTGTACTACTGTGCGCGGG |
| | GCATGTACGGATCCGTGCCGTACAAGGAGGGATACTACTTCGACATTTGGG |
| | GCCAGGGGACTCTTGTCACCGTGTCCTCG (SEQ ID NO: 2647) |
| Heavy Chain | QVQLQQSGPGLVKPSQTLSLTCAISGDSVSSSSAAWNWIRQSPSRGLEWLGHI |
| | GYRSKWYNEYAVSVKSRITINPDTSKNQFSLQLNSVTPEDTAVYYCARGMYG |
| | SVPYKEGYYFDIWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLYITR |
| | EPEVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKLTV |
| | DKSRWQQGNVFSCSVMHEALHNHYTQKSLSLSPGK (SEQ ID NO: 2658) |
| DNA Heavy | CAAGTGCAACTCCAGCAGTCAGGACCGGGGTTGGTCAAGCCTTCGCAGAC |
| Chain | CCTGTCCCTCACTTGCGCCATTAGCGGAGATTCGGTGTCGTCGTCGTCAGC |
| | CGCCTGGAACTGGATTAGACAGTCCCCTTCCCGAGGGCTGGAGTGGCTGGG |
| | CCACATCGGATACCGCAGCAAGTGGTACAACGAATACGCCGTCAGCGTGA |
| | AGTCACGCATCACCATCAACCCGGATACTAGCAAGAACCAGTTCAGCCTCC |
| | AGTTGAACTCCGTGACCCCGGAGGATACCGCCGTGTACTACTGTGCGCGGG |
| | GCATGTACGGATCCGTGCCGTACAAGGAGGGATACTACTTCGACATTTGGG |
| | GCCAGGGGACTCTTGTCACCGTGTCCTCGGCCTCCACTAAGGGCCCGTCAG |
| | TGTTCCCCCTTGCGCCATCCTCGAAGTCAACCTCCGGAGGAACTGCCGCAC |
| | TGGGTTGCCTCGTGAAAGACTATTTCCCGGAACCCGTCACTGTCTCCTGGA |
| | ACTCAGGAGCGCTCACCAGCGGAGTGCATACCTTTCCTGCGGTGCTGCAGT |
| | CCAGCGGCCTGTACTCCCTGAGCTCCGTCGTGACCGTCCCCTCGTCGTCCCT |
| | GGGAACCCAAACCTACATTTGCAACGTCAATCACAAGCCAAGCAACACTA |
| | AGGTGGACAAGAGAGTGGAGCCCAAGTCCTGCGATAAGACCCACACCTGT |
| | CCTCCCTGTCCGGCACCTGAACTGCTTGGTGGACCTTCCGTGTTCCTGTTCC |
| | CGCCCAAGCCAAAAGACACCCTGTATATCACTCGCGAACCGGAAGTCACTT |
| | GCGTGGTCGTGGACGTGTCCCACGAGGACCCCGAGGTCAAGTTTAATTGGT |
| | ACGTGGACGGAGTGGAAGTGCACAACGCCAAGACCAAGCCGCGGGAAGA |
| | ACAGTACAACTCCACCTACCGCGTGGTGTCCGTCCTGACTGTGCTCCACCA |
| | GGACTGGCTGAACGGAAAGGAGTACAAGTGCAAAGTGTCCAACAAGGCAC |
| | TGCCAGCCCCTATCGAAAAGACTATCTCCAAGGCCAAGGGCCAACCTAGG |
| | GAGCCCCAGGTGTACACGTTGCCTCCTTCCCGCGAAGAAATGACTAAGAAC |
| | CAGGTGTCGCTGACCTGTCTCGTGAAAGGGTTCTACCCCTCTGACATCGCC |
| | GTGGAATGGGAGTCAAACGGACAGCCTGAGAACAACTATAAGACCACACC |
| | ACCTGTCCTGGACTCCGACGGCTCCTTCTTCCTGTACTCAAAGTTGACCGTG |
| | GACAAGTCGCGGTGGCAACAGGGCAACGTGTTCTCTTGCTCCGTGATGCAC |
| | GAAGCCCTGCACAACCACTACACCCAAAAGTCGCTCAGCCTCTCCCCCGGA |
| | AAG (SEQ ID NO: 2659) |
| LCDR1 | RASQGISSDLN (SEQ ID NO: 2636) |
| (Combined) | |
| LCDR2 | AASNLQS (SEQ ID NO: 2637) |
| (Combined) | |
| LCDR3 | QQYTDESMT (SEQ ID NO: 2638) |
| (Combined) | |
| LCDR1 | RASQGISSDLN (SEQ ID NO: 2636) |
| (Kabat) | |
| LCDR2 | AASNLQS (SEQ ID NO: 2637) |
| (Kabat) | |
| LCDR3 | QQYTDESMT (SEQ ID NO: 2638) |
| (Kabat) | |
| LCDR1 | SQGISSD (SEQ ID NO: 2639) |
| (Chothia) | |
| LCDR2 | AAS (SEQ ID NO: 2640) |
| (Chothia) | |
| LCDR3 | YTDESM (SEQ ID NO: 2641) |
| (Chothia) | |
| LCDR1 | QGISSD (SEQ ID NO: 2642) |
| (IMGT) | |
| LCDR2 | AAS (SEQ ID NO: 2640) |
| (IMGT) | |
| LCDR3 | QQYTDESMT (SEQ ID NO: 2638) |
| (IMGT) | |
| VL | DIQMTQSPSSLSASVGDRVTITCRASQGISSDLNWYQQKPGKAPKLLIYAASNL |
| | QSGVPSRFSGSGSGTDFTLTISSLQPEDFATYYCQQYTDESMTFGQGTKVEIK |
| | (SEQ ID NO: 2643) |
| DNA VL | GACATTCAGATGACCCAGTCCCCGTCGTCCCTGTCCGCATCCGTGGGCGAC |
| | AGAGTCACCATCACTTGCCGGGCCTCACAGGGAATTTCCTCCGACCTGAAC |
| | TGGTATCAGCAGAAGCCTGGAAAGGCCCCGAAGCTGCTGATCTACGCCGC |
| | GTCCAACTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCCAACAGTACACAGATGAATCCATGACCTTCGGACAGGGCAC |
| | CAAAGTCGAGATCAAG (SEQ ID NO: 2650) |
| Light Chain | DIQMTQSPSSLSASVGDRVTITCRASQGISSDLNWYQQKPGKAPKLLIYAASNL |
| | QSGVPSRFSGSGSGTDFTLTISSLQPEDFATYYCQQYTDESMTFGQGTKVEIKR |
| | TVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 2645) |
| DNA Light | GACATTCAGATGACCCAGTCCCCGTCGTCCCTGTCCGCATCCGTGGGCGAC |
| Chain | AGAGTCACCATCACTTGCCGGGCCTCACAGGGAATTTCCTCCGACCTGAAC |
| | TGGTATCAGCAGAAGCCTGGAAAGGCCCCGAAGCTGCTGATCTACGCCGC |
| | GTCCAACTTGCAATCGGGAGTGCCAAGCCGCTTTTCTGGTTCCGGGAGCGG |
| | GACTGACTTCACCCTGACTATTAGCAGCCTGCAGCCCGAAGATTTCGCTAC |
| | CTACTACTGCCAACAGTACACAGATGAATCCATGACCTTCGGACAGGGCAC |
| | CAAAGTCGAGATCAAGCGTACGGTGGCCGCTCCCAGCGTGTTCATCTTCCC |
| | CCCCAGCGACGAGCAGCTGAAGAGCGGCACCGCCAGCGTGGTGTGCCTGC |
| | TGAACAACTTCTACCCCCGGGAGGCCAAGGTGCAGTGGAAGGTGGACAAC |
| | GCCCTGCAGAGCGGCAACAGCCAGGAGAGCGTCACCGAGCAGGACAGCA |
| | AGGACTCCACCTACAGCCTGAGCAGCACCCTGACCCTGAGCAAGGCCGAC |
| | TACGAGAAGCATAAGGTGTACGCCTGCGAGGTGACCCACCAGGGCCTGTC |
| | CAGCCCCGTGACCAAGAGCTTCAACAGGGGCGAGTGC (SEQ ID NO: 2651) |

MOR042596

| | |
|---|---|
| HCDR1 | GYTFTGYHMS (SEQ ID NO: 2586) |
| (Combined) | |
| HCDR2 | VINPVSGNTVYAQKFQG (SEQ ID NO: 2587) |
| (Combined) | |
| HCDR3 | IPSYTYAFDY (SEQ ID NO: 2588) |
| (Combined) | |
| HCDR1 | GYHMS (SEQ ID NO: 2589) |
| (Kabat) | |
| HCDR2 | VINPVSGNTVYAQKFQG (SEQ ID NO: 2587) |
| (Kabat) | |
| HCDR3 | IPSYTYAFDY (SEQ ID NO: 2588) |
| (Kabat) | |
| HCDR1 | GYTFTGY (SEQ ID NO: 2590) |
| (Chothia) | |
| HCDR2 | NPVSGN (SEQ ID NO: 2591) |
| (Chothia) | |
| HCDR3 | IPSYTYAFDY (SEQ ID NO: 2588) |
| (Chothia) | |
| HCDR1 | GYTFTGYH (SEQ ID NO: 2592) |
| (IMGT) | |
| HCDR2 | INPVSGNT (SEQ ID NO: 2593) |
| (IMGT) | |
| HCDR3 | ARIPSYTYAFDY (SEQ ID NO: 2594) |
| (IMGT) | |
| VH | QVQLVQSGAEVKKPGASVKVSCKASGYTFTGYHMSWVRQAPGQGLEWMGV |
| | INPVSGNTVYAQKFQGRVTMTRDTSISTAYMELSRLRSEDTAVYYCARIPSYT |
| | YAFDYWGQGTLVTVSS (SEQ ID NO: 2595) |
| DNA VH | CAGGTGCAATTGGTGCAGAGCGGTGCGGAAGTGAAAAAACCGGGTGCCAG |
| | CGTGAAAGTTAGCTGCAAAGCGTCCGGATATACCTTCACTGGTTACCATAT |
| | GTCTTGGGTGCGCCAGGCCCCGGGCCAGGGCCTCGAGTGGATGGGCGTTAT |
| | CAACCCGGTTTCTGGCAACACGGTTTACGCGCAGAAATTTCAGGGCCGGGT |
| | GACCATGACCCGTGATACCAGCATTAGCACCGCGTATATGGAACTGAGCC |
| | GTCTGCGTAGCGAAGATACGGCCGTGTATTATTGCGCGCGTATCCCGTCTT |
| | ACACTTACGCTTTCGATTACTGGGGCCAAGGCACCCTGGTGACTGTTAGCT |
| | CA (SEQ ID NO: 2596) |
| Heavy Chain | QVQLVQSGAEVKKPGASVKVSCKASGYTFTGYHMSWVRQAPGQGLEWMGV |
| | INPVSGNTVYAQKFQGRVTMTRDTSISTAYMELSRLRSEDTAVYYCARIPSYT |
| | YAFDYWGQGTLVTVSSASTKGPSVFPLAPSSKSTSGGTAALGCLVKDYFPEPV |
| | TVSWNSGALTSGVHTFPAVLQSSGLYSLSSVVTVPSSSLGTQTYICNVNHKPSN |
| | TKVDKRVEPKSCDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSREEMTKNQVSL |
| | TCLVKGFYPSDIAVEWESNGQPENNYKTTPPVLDSDGSFFLYSKLTVDKSRWQ |
| | QGNVFSCSVMHEALHNHYTQKSLSLSPGK (SEQ ID NO: 2597) |
| DNA Heavy | CAGGTGCAATTGGTGCAGAGCGGTGCGGAAGTGAAAAAACCGGGTGCCAG |
| Chain | CGTGAAAGTTAGCTGCAAAGCGTCCGGATATACCTTCACTGGTTACCATAT |
| | GTCTTGGGTGCGCCAGGCCCCGGGCCAGGGCCTCGAGTGGATGGGCGTTAT |
| | CAACCCGGTTTCTGGCAACACGGTTTACGCGCAGAAATTTCAGGGCCGGGT |
| | GACCATGACCCGTGATACCAGCATTAGCACCGCGTATATGGAACTGAGCC |
| | GTCTGCGTAGCGAAGATACGGCCGTGTATTATTGCGCGCGTATCCCGTCTT |
| | ACACTTACGCTTTCGATTACTGGGGCCAAGGCACCCTGGTGACTGTTAGCT |
| | CAGCCTCCACCAAGGGTCCATCGGTCTTCCCCCTGGCACCCTCCTCCAAGA |
| | GCACCTCTGGGGGCACAGCGGCCCTGGGCTGCCTGGTCAAGGACTACTTCC |
| | CCGAACCGGTGACGGTGTCGTGGAACTCAGGCGCCCTGACCAGCGGCGTG |
| | CACACCTTCCCGGCTGTCCTACAGTCCTCAGGACTCTACTCCCTCAGCAGC |
| | GTGGTGACCGTGCCCTCCAGCAGCTTGGGCACCCAGACCTACATCTGCAAC |
| | GTGAATCACAAGCCCAGCAACACCAAGGTGGACAAGAGAGTTGAGCCCAA |
| | ATCTTGTGACAAAACTCACACATGCCCACCGTGCCCAGCACCTGAAGCAGC |
| | GGGGGGACCGTCAGTCTTCCTCTTCCCCCCAAAACCCAAGGACACCCTCAT |
| | GATCTCCCGGACCCCTGAGGTCACATGCGTGGTGGTGGACGTGAGCCACG |
| | AAGACCCTGAGGTCAAGTTCAACTGGTACGTGGACGGCGTGGAGGTGCAT |
| | AATGCCAAGACAAAGCCGCGGGAGGAGCAGTACAACAGCACGTACCGGGT |
| | GGTCAGCGTCCTCACCGTCCTGCACCAGGACTGGCTGAATGGCAAGGAGT |
| | ACAAGTGCAAGGTCTCCAACAAAGCCCTCCCAGCCCCCATCGAGAAAACC |
| | ATCTCCAAAGCCAAAGGGCAGCCCCGAGAACCACAGGTGTACACCCTGCC |
| | CCCATCCCGGGAGGAGATGACCAAGAACCAGGTCAGCCTGACCTGCCTGG |
| | TCAAAGGCTTCTATCCCAGCGACATCGCCGTGGAGTGGGAGAGCAATGGG |
| | CAGCCGGAGAACAACTACAAGACCACGCCTCCCGTGCTGGACTCCGACGG |
| | CTCCTTCTTCCTCTACAGCAAGCTCACCGTGGACAAGAGCAGGTGGCAGCA |
| | GGGGAACGTCTTCTCATGCTCCGTGATGCATGAGGCTCTGCACAACCACTA |
| | CACGCAGAAGAGCCTCTCCCTGTCTCCGGGTAAA (SEQ ID NO: 2598) |
| LCDR1 | RASQDISNYLA (SEQ ID NO: 2599) |
| (Combined) | |
| LCDR2 | RASSLQS (SEQ ID NO: 2600) |
| (Combined) | |
| LCDR3 | QQHGHSPTT (SEQ ID NO: 2660) |
| (Combined) | |
| LCDR1 | RASQDISNYLA (SEQ ID NO: 2599) |
| (Kabat) | |
| LCDR2 | RASSLQS (SEQ ID NO: 2600) |
| (Kabat) | |
| LCDR3 | QQHGHSPTT (SEQ ID NO: 2660) |
| (Kabat) | |
| LCDR1 | SQDISNY (SEQ ID NO: 2602) |
| (Chothia) | |
| LCDR2 | RAS (SEQ ID NO: 2603) |
| (Chothia) | |
| LCDR3 | HGHSPT (SEQ ID NO: 2661) |
| (Chothia) | |
| LCDR1 | QDISNY (SEQ ID NO: 2605) |
| (IMGT) | |
| LCDR2 | RAS (SEQ ID NO: 2603) |
| (IMGT) | |
| LCDR3 | QQHGHSPTT (SEQ ID NO: 2660) |
| (IMGT) | |
| VL | DIQMTQSPSSLSASVGDRVTITCRASQDISNYLAWYQQKPGKAPKLLIYRASSL |
| | QSGVPSRFSGSGSGTDFTLTISSLQPEDFATYYCQQHGHSPTTFGQGTKVEIK |
| | (SEQ ID NO: 2662) |
| DNA VL | GATATCCAGATGACCCAGAGCCCGAGCAGCCTGAGCGCCAGCGTGGGCGA |
| | TCGCGTGACCATTACCTGCAGAGCCAGCCAGGACATTTCTAACTACCTGGC |
| | TTGGTACCAGCAGAAACCGGGCAAAGCGCCGAAACTATTAATCTACCGTG |
| | CTTCTTCTCTGCAAAGCGGCGTGCCGAGCCGCTTTAGCGGCAGCGGATCCG |
| | GCACCGATTTCACCCTGACCATTAGCTCTCTGCAACCGGAAGACTTTGCGA |
| | CCTATTATTGCCAGCAGCATGGTCATTCTCCGACTACCTTTGGCCAGGGCA |
| | CGAAAGTTGAAATTAAA (SEQ ID NO: 2663) |
| Light Chain | DIQMTQSPSSLSASVGDRVTITCRASQDISNYLAWYQQKPGKAPKLLIYRASSL |
| | QSGVPSRFSGSGSGTDFTLTISSLQPEDFATYYCQQHGHSPTTFGQGTKVEIKRT |
| | VAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 2664) |
| DNA Light | GATATCCAGATGACCCAGAGCCCGAGCAGCCTGAGCGCCAGCGTGGGCGA |
| Chain | TCGCGTGACCATTACCTGCAGAGCCAGCCAGGACATTTCTAACTACCTGGC |
| | TTGGTACCAGCAGAAACCGGGCAAAGCGCCGAAACTATTAATCTACCGTG |
| | CTTCTTCTCTGCAAAGCGGCGTGCCGAGCCGCTTTAGCGGCAGCGGATCCG |
| | GCACCGATTTCACCCTGACCATTAGCTCTCTGCAACCGGAAGACTTTGCGA |
| | CCTATTATTGCCAGCAGCATGGTCATTCTCCGACTACCTTTGGCCAGGGCA |
| | CGAAAGTTGAAATTAAACGTACGGTGGCCGCTCCCAGCGTGTTCATCTTCC |
| | CCCCCAGCGACGAGCAGCTGAAGAGCGGCACCGCCAGCGTGGTGTGCCTG |
| | CTGAACAACTTCTACCCCCGGGAGGCCAAGGTGCAGTGGAAGGTGGACAA |
| | CGCCCTGCAGAGCGGCAACAGCCAGGAAAGCGTCACCGAGCAGGACAGCA |
| | AGGACTCCACCTACAGCCTGAGCAGCACCCTGACCCTGAGCAAGGCCGAC |
| | TACGAGAAGCACAAGGTGTACGCCTGCGAGGTGACCCACCAGGGCCTGTC |
| | CAGCCCCGTGACCAAGAGCTTCAACCGGGGCGAGTGT (SEQ ID NO: 2665) |

MOR041877

| | |
|---|---|
| HCDR1 | GFSLSTSGVGVS (SEQ ID NO: 2666) |
| (Combined) | |
| HCDR2 | LIFSDHDKIYSTSLKT (SEQ ID NO: 2667) |
| (Combined) | |
| HCDR3 | TLIDRSVYFDY (SEQ ID NO: 2668) |
| (Combined) | |
| HCDR1 | TSGVGVS (SEQ ID NO: 2669) |
| (Kabat) | |
| HCDR2 | LIFSDHDKIYSTSLKT (SEQ ID NO: 2667) |
| (Kabat) | |
| HCDR3 | TLIDRSVYFDY (SEQ ID NO: 2668) |
| (Kabat) | |
| HCDR1 | GFSLSTSGV (SEQ ID NO: 2670) |
| (Chothia) | |
| HCDR2 | FSDHD (SEQ ID NO: 2671) |
| (Chothia) | |
| HCDR3 | TLIDRSVYFDY (SEQ ID NO: 2668) |
| (Chothia) | |
| HCDR1 | GFSLSTSGVG (SEQ ID NO: 2672) |
| (IMGT) | |
| HCDR2 | IFSDHDK (SEQ ID NO: 2673) |
| (IMGT) | |
| HCDR3 | ARTLIDRSVYFDY (SEQ ID NO: 2674) |
| (IMGT) | |
| VH | QVQLKESGPALVKPTQTLTLTCTF SGF SLSTSGVGVSWIRQPPGKALEWLALIF |
| | SDHDKIYSTSLKTRLTISKDTSKNQVVLTMTNMDPVDTATYYCARTLIDRSVY |
| | FDYWGQGTLVTVSS (SEQ ID NO: 2675) |
| DNA VH | CAGGTGCAATTGAAAGAAAGCGGTCCGGCGCTGGTGAAACCGACCCAGAC |
| | CCTGACCCTGACGTGCACCTTTTCCGGATTCAGCCTGTCTACTTCCGGTGTT |
| | GGTGTGAGCTGGATTCGCCAGCCGCCGGGCAAAGCGCTCGAGTGGCTGGC |
| | GCTGATCTTCTCTGACCATGACAAGATCTATAGCACCAGCCTGAAAACCCG |
| | TCTGACCATTAGCAAAGATACTTCGAAAAACCAGGTGGTGCTGACCATGAC |
| | CAACATGGACCCGGTGGATACCGCGACCTATTATTGCGCGCGTACTCTGAT |
| | CGACCGTTCTGTTTACTTCGATTACTGGGGCCAAGGCACCCTGGTGACTGTT |
| | AGCTCA (SEQ ID NO: 2676) |
| Heavy Chain | QVQLKESGPALVKPTQ TLTLTCTF SGF SLSTSGVGVSWIRQPPGKALEWLALIF |
| | SDHDKIYSTSLKTRLTISKDTSKNQVVLTMTNMDPVDTATYYCARTLIDRSVY |
| | FDYWGQGTLVTVSSASTKGPSVFPLAPSSKSTSGGTAALGCLVKDYFPEPVTV |
| | SWNSGALTSGVHTFPAVLQSSGLYSLSSVVTVPSSSLGTQTYICNVNHKPSNTK |
| | VDKRVEPKSCDKTHTCPPCPAPEAAGGPSVFLFPPKPKDTLMISRTPEVTCVVV |
| | DVSHEDPEVKFNWYVDGVEVHNAKTKPREEQYNSTYRVVSVLTVLHQDWLN |
| | GKEYKCKVSNKALPAPIEKTISKAKGQPREPQVYTLPPSREEMTKNQVSLTCL |
| | VKGFYPSDIAVEWESNGQPENNYKTTPPVLDSDGSFFLYSKLTVDKSRWQQG |
| | NVFSCSVMHEALHNHYTQKSLSLSPGK (SEQ ID NO: 2677) |
| DNA Heavy | CAGGTGCAATTGAAAGAAAGCGGTCCGGCGCTGGTGAAACCGACCCAGAC |
| Chain | CCTGACCCTGACGTGCACCTTTTCCGGATTCAGCCTGTCTACTTCCGGTGTT |
| | GGTGTGAGCTGGATTCGCCAGCCGCCGGGCAAAGCGCTCGAGTGGCTGGC |
| | GCTGATCTTCTCTGACCATGACAAGATCTATAGCACCAGCCTGAAAACCCG |
| | TCTGACCATTAGCAAAGATACTTCGAAAAACCAGGTGGTGCTGACCATGAC |
| | CAACATGGACCCGGTGGATACCGCGACCTATTATTGCGCGCGTACTCTGAT |
| | CGACCGTTCTGTTTACTTCGATTACTGGGGCCAAGGCACCCTGGTGACTGTT |
| | AGCTCAGCCTCCACCAAGGGTCCATCGGTCTTCCCCCTGGCACCCTCCTCC |
| | AAGAGCACCTCTGGGGGCACAGCGGCCCTGGGCTGCCTGGTCAAGGACTA |
| | CTTCCCCGAACCGGTGACGGTGTCGTGGAACTCAGGCGCCCTGACCAGCGG |
| | CGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGC |
| | AACGTGAATCACAAGCCCAGCAACACCAAGGTGGACAAGAGAGTTGAGCC |
| | CAAATCTTGTGACAAAACTCACACATGCCCACCGTGCCCAGCACCTGAAGC |
| | AGCGGGGGGACCGTCAGTCTTCCTCTTCCCCCCAAAACCCAAGGACACCCT |
| | CATGATCTCCCGGACCCCTGAGGTCACATGCGTGGTGGTGGACGTGAGCCA |
| | CGAAGACCCTGAGGTCAAGTTCAACTGGTACGTGGACGGCGTGGAGGTGC |
| | ATAATGCCAAGACAAAGCCGCGGGAGGAGCAGTACAACAGCACGTACCGG |
| | GTGGTCAGCGTCCTCACCGTCCTGCACCAGGACTGGCTGAATGGCAAGGA |
| | GTACAAGTGCAAGGTCTCCAACAAAGCCCTCCCAGCCCCCATCGAGAAAA |
| | CCATCTCCAAAGCCAAAGGGCAGCCCCGAGAACCACAGGTGTACACCCTG |
| | CCCCCATCCCGGGAGGAGATGACCAAGAACCAGGTCAGCCTGACCTGCCT |
| | GGTCAAAGGCTTCTATCCCAGCGACATCGCCGTGGAGTGGGAGAGCAATG |
| | GGCAGCCGGAGAACAACTACAAGACCACGCCTCCCGTGCTGGACTCCGAC |
| | GGCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCAC |
| | TACACGCAGAAGAGCCTCTCCCTGTCTCCGGGTAAA (SEQ ID NO: 2678) |
| LCDR1 | SGSSSNIGHHYVS (SEQ ID NO: 2679) |
| (Combined) | |
| LCDR2 | DNTNRPS (SEQ ID NO: 2680) |
| (Combined) | |
| LCDR3 | ATWDGLMNSIV (SEQ ID NO: 2681) |
| (Combined) | |
| LCDR1 | SGSSSNIGHHYVS (SEQ ID NO: 2679) |
| (Kabat) | |
| LCDR2 | DNTNRPS (SEQ ID NO: 2680) |
| (Kabat) | |
| LCDR3 | ATWDGLMNSIV (SEQ ID NO: 2681) |
| (Kabat) | |
| LCDR1 | SSSNIGHHY (SEQ ID NO: 2682) |
| (Chothia) | |
| LCDR2 | DNT (SEQ ID NO: 2683) |
| (Chothia) | |
| LCDR3 | WDGLMNSI (SEQ ID NO: 2684) |
| (Chothia) | |
| LCDR1 | SSNIGHHY (SEQ ID NO: 2685) |
| (IMGT) | |
| LCDR2 | DNT (SEQ ID NO: 2683) |
| (IMGT) | |
| LCDR3 | ATWDGLMNSIV (SEQ ID NO: 2681) |
| (IMGT) | |
| VL | DIVLTQPPSVSGAPGQRVTISCSGSSSNIGHHYVSWYQQLPGTAPKLLIYDNTN |
| | RPSGVPDRFSGSKSGTSASLAITGLQAEDEADYYCATWDGLMNSIVFGGGTKL |
| | TVL (SEQ ID NO: 2686) |
| DNA VL | GATATCGTGCTGACCCAGCCGCCGAGCGTGAGCGGTGCACCGGGCCAGCG |
| | CGTGACCATTAGCTGTAGCGGCAGCAGCAGCAACATTGGTCATCATTACGT |
| | GTCTTGGTACCAGCAGCTGCCGGGCACGGCGCCGAAACTGCTGATCTACGA |
| | CAACACTAACCGCCCGAGCGGCGTGCCGGATCGCTTTAGCGGATCCAAAA |
| | GCGGCACCAGCGCCAGCCTGGCGATTACCGGCCTGCAAGCAGAAGACGAA |
| | GCGGATTATTACTGCGCTACTTGGGACGGTCTGATGAACTCTATCGTGTTTG |
| | GCGGCGGCACGAAGTTAACCGTCCTA (SEQ ID NO: 2687) |
| Light Chain | DIVLTQPPSVSGAPGQRVTISCSGSSSNIGHHYVSWYQQLPGTAPKLLIYDNTN |
| | RPSGVPDRFSGSKSGTSASLAITGLQAEDEADYYCATWDGLMNSIVFGGGTKL |
| | TVLGQPKAAPSVTLFPPSSEELQANKATLVCLISDFYPGAVTVAWKADSSPVK |
| | AGVETTTPSKQSNNKYAASSYLSLTPEQWKSHRSYSCQVTHEGSTVEKTVAPT |
| | ECS (SEQ ID NO: 2688) |
| DNA Light | GATATCGTGCTGACCCAGCCGCCGAGCGTGAGCGGTGCACCGGGCCAGCG |
| Chain | CGTGACCATTAGCTGTAGCGGCAGCAGCAGCAACATTGGTCATCATTACGT |
| | GTCTTGGTACCAGCAGCTGCCGGGCACGGCGCCGAAACTGCTGATCTACGA |
| | CAACACTAACCGCCCGAGCGGCGTGCCGGATCGCTTTAGCGGATCCAAAA |
| | GCGGCACCAGCGCCAGCCTGGCGATTACCGGCCTGCAAGCAGAAGACGAA |
| | GCGGATTATTACTGCGCTACTTGGGACGGTCTGATGAACTCTATCGTGTTTG |
| | GCGGCGGCACGAAGTTAACCGTCCTAGGTCAGCCCAAGGCTGCCCCCTCG |
| | GTCACTCTGTTCCCGCCCTCCTCTGAGGAGCTTCAAGCCAACAAGGCCACA |
| | CTGGTGTGTCTCATAAGTGACTTCTACCCGGGAGCCGTGACAGTGGCCTGG |
| | AAGGCAGATAGCAGCCCCGTCAAGGCGGGAGTGGAGACCACCACACCCTC |
| | CAAACAAAGCAACAACAAGTACGCGGCCAGCAGCTATCTGAGCCTGACGC |
| | CTGAGCAGTGGAAGTCCCACAGAAGCTACAGCTGCCAGGTCACGCATGAA |
| | GGGAGCACCGTGGAGAAGACAGTGGCCCCTACAGAATGTTCA |
| | (SEQ ID NO: 2689) |

In some embodiments, each of the light chain variable regions and each of the heavy chain variable regions disclosed above, including those in TABLE J1 above, may be attached to the light chain constant regions (TABLE EN1) and heavy chain constant regions (TABLE EN2) to form complete antibody light and heavy chains, respectively, as further discussed below. Further, each of the generated heavy and light chain sequences may be combined to form a complete antibody structure. It should be understood that the heavy chain and light chain variable regions provided herein can also be attached to other constant domains having different sequences than the exemplary sequences listed herein.

K. KR Patent Application Publication No. KR20200048069A

In some embodiments, the TREM2 agonist is an antibody, or an antigen-binding fragment thereof, as described in KR Patent Application Publication No. KR20200048069A, which is incorporated by reference herein, in its entirety.

In some embodiments, the TREM2 antibody comprises the CDR L1, CDR L2 and CDR L3 in the light chain variable region of the antibody produced by hybridoma cells with accession number KCTC 13471BP or hybridoma cells with accession number KTC 13470BP.

In some embodiments, the TREM2 antibody comprises the CDR H1, CDR H2 and CDR H3 in the heavy chain variable region of the antibody produced by hybridoma cells with accession number KCTC 13471BP or hybridoma cells with accession number KTC 13470BP.

In some embodiments, the TREM2 antibody comprises the CDR L1, CDR L2 and CDR L3 in the light chain variable region and the CDR H1, CDR H2 and CDR H3 in the heavy chain variable region of the antibody produced by hybridoma cells with accession number KCTC 13471BP or hybridoma cells with accession number KTC 13470BP.

In some embodiments, the TREM2 antibody comprises the light chain variable region and the heavy chain variable region of the antibody produced by hybridoma cells with accession number KCTC 13471BP or hybridoma cells with accession number KTC 13470BP.

In some embodiments, the TREM2 agonist is an antibody produced by hybridoma cells with accession number KCTC 13471BP or hybridoma cells with accession number KTC 13470BP.

In some embodiments, the light chain variable regions and the heavy chain variable regions describe above for the antibody produced by hybridoma cells with accession number KCTC 13471BP or hybridoma cells with accession number KTC 13470BP may be attached to the light chain constant regions (TABLE EN1) and heavy chain constant regions (TABLE EN2) to form complete antibody light and heavy chains, respectively, as further discussed below. Further, each of the generated heavy and light chain sequences may be combined to form a complete antibody structure. It should be understood that the heavy chain and light chain variable regions provided herein can also be attached to other constant domains having different sequences than the exemplary sequences listed herein.

L. PCT Patent Application Publication No. WO2020/172450A1

In some embodiments, the TREM2 agonist is an antibody, or an antigen-binding fragment thereof, as described in PCT Patent Application Publication No. WO2020/172450A1 (“the '450 application”), which is incorporated by reference herein, in its entirety.

In some embodiments, the antibody or antigen-binding fragment thereof comprises:
(a) a CDR-H1 sequence comprising the sequence of GFSIEDFYIH (SEQ ID NO:2717);
(b) a CDR-H2 sequence comprising the sequence of W-I-D-P-E-β₆-G-β₈-S-K-Y-A-P-K-F-Q-G (SEQ ID NO:2735), wherein β₆ is N or Q and β₈ is D or E;
(c) a CDR-H3 sequence comprising the sequence of HADHGNYGSTMDY (SEQ ID NO:2719);
(d) CDR-L1 sequence comprising the sequence of HASQHINVWLS (SEQ ID NO:2720);
(e) a CDR-L2 sequence comprising the sequence of KASNLHT (SEQ ID NO:2721); and
(f) a CDR-L3 sequence comprising the sequence of QQGQTYPRT (SEQ ID NO: 2722).

In some embodiments, the CDR-H2 sequence is selected from SEQ ID NOS:2718, 2727, 2729, and 2731.

In some embodiments, the antibody or antigen-binding fragment comprises:
(a) a CDR-H1 comprising the amino acid sequence of SEQ ID NO:2717, a CDR-H2 comprising the amino acid sequence of SEQ ID NO:2718, a CDR-H3 comprising the amino acid sequence of SEQ ID NO:2719, a CDR-L1 comprising the amino acid sequence of SEQ ID NO:2720, a CDR-L2 comprising the amino acid sequence of SEQ ID NO:2721, and a CDR-L3 comprising the amino acid sequence of SEQ ID NO:2722; or
(b) a CDR-H1 comprising the amino acid sequence of SEQ ID NO:2717, a CDR-H2 comprising the amino acid sequence of SEQ ID NO:2727, a CDR-H3 comprising the amino acid sequence of SEQ ID NO:2719, a CDR-L1 comprising the amino acid sequence of SEQ ID NO:2720, a CDR-L2 comprising the amino acid sequence of SEQ ID NO:2721, and a CDR-L3 comprising the amino acid sequence of SEQ ID NO:2722; or
(c) a CDR-H1 comprising the amino acid sequence of SEQ ID NO:2717, a CDR-H2 comprising the amino acid sequence of SEQ ID NO:2729, a CDR-H3 comprising the amino acid sequence of SEQ ID NO:2719, a CDR-L1 comprising the amino acid sequence of SEQ ID NO:2720, a CDR-L2 comprising the amino acid sequence of SEQ ID NO:2721, and a CDR-L3 comprising the amino acid sequence of SEQ ID NO:2722; or
(d) a CDR-H1 comprising the amino acid sequence of SEQ ID NO:2717, a CDR-H2 comprising the amino acid sequence of SEQ ID NO:2731, a CDR-H3 comprising the amino acid sequence of SEQ ID NO:2719, a CDR-L1 comprising the amino acid sequence of SEQ ID NO:2720, a CDR-L2 comprising the amino acid sequence of SEQ ID NO:2721, and a CDR-L3 comprising the amino acid sequence of SEQ ID NO:2722.

In some embodiments, the antibody or antigen-binding fragment comprises a V H sequence that has at least 85% sequence identity to any one of SEQ ID NOS:2715, 2723, 2725, 2726, 2728, 2730, 2732, 2733, and 2734. In some embodiments, the V H sequence has at least 90% sequence identity to SEQ ID NO:2715. In some embodiments, the V_{H} sequence has at least 95% sequence identity to SEQ ID NO:2715. In some embodiments, the VH sequence comprises SEQ ID NO:2715. In some embodiments, the V_{H} sequence has at least 90% sequence identity to SEQ ID NO:2730. In some embodiments, the V_{H} sequence has at least 95% sequence identity to SEQ ID NO:2730. In some embodiments, the V_{H} sequence comprises SEQ ID NO:2730. In some embodiments, the V_{H} sequence has at least 90% sequence identity to SEQ ID NO:2733. In some embodiments, the V_{H} sequence has at least 95% sequence identity to SEQ ID NO:2733. In some embodiments, the V_{H} sequence comprises SEQ ID NO:2733.

In some embodiments, the antibody or antigen-binding fragment comprises a V_{L} sequence that has at least 85% sequence identity to SEQ ID NO:2716 or SEQ ID NO:2724. In some embodiments, the V_{L} sequence has at least 90% sequence identity to SEQ ID NO:2716. In some embodiments, the V_{L}sequence has at least 95% sequence identity to SEQ ID NO:2716. In some embodiments, the V_{L}sequence comprises SEQ ID NO:2716. In some embodiments, the V_{L} sequence has at least 90% sequence identity to SEQ ID NO:2724. In some embodiments, the V_{L} sequence has at least 95% sequence identity to SEQ ID NO:2724. In some embodiments, the V_{L} sequence comprises SEQ ID NO:2724.

In some embodiments, the antibody or antigen-binding fragment comprises:
(a) a VH sequence comprising SEQ ID NO:2715 and a V_{L} sequence comprising SEQ ID NO:2716; or
(b) a VH sequence comprising SEQ ID NO:2723 and a V_{L} sequence comprising SEQ ID NO:2724; or
(c) a VH sequence comprising SEQ ID NO:2725 and a V_{L} sequence comprising SEQ ID NO:2724; or
(d) a VH sequence comprising SEQ ID NO:2726 and a V_{L} sequence comprising SEQ ID NO:2724; or
(e) a VH sequence comprising SEQ ID NO:2728 and a V_{L} sequence comprising SEQ ID NO:2724; or
(f) a VH sequence comprising SEQ ID NO:2730 and a V_{L} sequence comprising SEQ ID NO:2724; or
(g) a VH sequence comprising SEQ ID NO:2732 and a V_{L} sequence comprising SEQ ID NO:2724; or
(h) a VH sequence comprising SEQ ID NO:2733 and a VL sequence comprising SEQ ID NO:2724; or
(i) a VH sequence comprising SEQ ID NO:2734 and a VL sequence comprising SEQ ID NO:2724.

In some embodiments, an antibody or antigen-binding fragment thereof that specifically binds to TREM2 comprises:
(a) a CDR-H1 sequence comprising the sequence of G-F-T-F-T-α₆-F-Y-M-S(SEQ ID NO:2736), wherein a₆ is D or N;
(b) a CDR-H2 sequence comprising the sequence of V-I-R-N-β₅-β₆-N-β₈-Y-T-β₁₁-β₁₂-Y-N-P-S-V-K-G (SEQ ID NO:2737), wherein β₅ is K or R; β₆ is A or P; β₈ is G or A; β₁₁ is A or T; and β₁₂ is G or D;
(c) a CDR-H3 sequence comprising the sequence of γ₁-R-L-γ₄-Y-G-F-D-Y (SEQ ID NO:2738), wherein γ₁ is A or T; and γ4 is T or S;
(d) a CDR-L1 sequence comprising the sequence of Q-S-S-K-S-L-L-H-S-δτo-G-K-T-Y-L-N (SEQ ID NO:2739), wherein &to is N or T; a CDR-L2 sequence comprising the sequence of WMSTRAS (SEQ ID NO: 2696); and
(e) a CDR-L3 sequence comprising the sequence of Q-Q-F-L-E-ϕ₆-P-F-T (SEQ ID NO:2740), wherein ϕ₆ is Y or F.

In some embodiments, the CDR-H1 sequence is selected from any one of SEQ ID NOS:2692 and 2700. In some embodiments, the CDR-H2 sequence is selected from any one of SEQ ID NOS:2693, 2701, and 2713. In some embodiments, the CDR-H3 sequence is selected from any one of SEQ ID NOS:2694, 2702, and 2705. In some embodiments, the CDR-L1 sequence is selected from any one of SEQ ID NOS:2695 and 2711. In some embodiments, the CDR-L3 sequence is selected from any one of SEQ ID NOS:2697 and 2706.

In some embodiments, the antibody or antigen-binding fragment comprises:
(a) a CDR-H1 comprising the amino acid sequence of SEQ ID NO:2692, a CDR-H2 comprising the amino acid sequence of SEQ ID NO:2693, a CDR-H3 comprising the amino acid sequence of SEQ ID NO:2705, a CDR-L1 comprising the amino acid sequence of SEQ ID NO:2695, a CDR-L2 comprising the amino acid sequence of SEQ ID NO:2696, and a CDR-L3 comprising the amino acid sequence of SEQ ID NO:2706; or
(b) a CDR-H1 comprising the amino acid sequence of SEQ ID NO:2692, a CDR-H2 comprising the amino acid sequence of SEQ ID NO:2693, a CDR-H3 comprising the amino acid sequence of SEQ ID NO:2705, a CDR-L1 comprising the amino acid sequence of SEQ ID NO:2711, a CDR-L2 comprising the amino acid sequence of SEQ ID NO:2696, and a CDR-L3 comprising the amino acid sequence of SEQ ID NO:2706; or
(c) a CDR-H1 comprising the amino acid sequence of SEQ ID NO:2692, a CDR-H2 comprising the amino acid sequence of SEQ ID NO:2713, a CDR-H3 comprising the amino acid sequence of SEQ ID NO:2705, a CDR-L1 comprising the amino acid sequence of SEQ ID NO:2695, a CDR-L2 comprising the amino acid sequence of SEQ ID NO:2696, and a CDR-L3 comprising the amino acid sequence of SEQ ID NO:2706; or
(d) a CDR-H1 comprising the amino acid sequence of SEQ ID NO:2692, a CDR-H2 comprising the amino acid sequence of SEQ ID NO:2713, a CDR-H3 comprising the amino acid sequence of SEQ ID NO:2705, a CDR-L1 comprising the amino acid sequence of SEQ ID NO:2711, a CDR-L2 comprising the amino acid sequence of SEQ ID NO:2696, and a CDR-L3 comprising the amino acid sequence of SEQ ID NO:2706; or
(e) a CDR-H1 comprising the amino acid sequence of SEQ ID NO:2692, a CDR-H2 comprising the amino acid sequence of SEQ ID NO:2693, a CDR-H3 comprising the amino acid sequence of SEQ ID NO:2694, a CDR-L1 comprising the amino acid sequence of SEQ ID NO:2695, a CDR-L2 comprising the amino acid sequence of SEQ ID NO:2696, and a CDR-L3 comprising the amino acid sequence of SEQ ID NO:2697; or
(f) a CDR-H1 comprising the amino acid sequence of SEQ ID NO:2700, a CDR-H2 comprising the amino acid sequence of SEQ ID NO:2701, a CDR-H3 comprising the amino acid sequence of SEQ ID NO:2702, a CDR-L1 comprising the amino acid sequence of SEQ ID NO:2695, a CDR-L2 comprising the amino acid sequence of SEQ ID NO:2696, and a CDR-L3 comprising the amino acid sequence of SEQ ID NO:2697; or
(g) a CDR-H1 comprising the amino acid sequence of SEQ ID NO:2692, a CDR-H2 comprising the amino acid sequence of SEQ ID NO:2713, a CDR-H3 comprising the amino acid sequence of SEQ ID NO:2705, a CDR-L1 comprising the amino acid sequence of SEQ ID NO:2695, a CDR-L2 comprising the amino acid sequence of SEQ ID NO:2696, and a CDR-L3 comprising the amino acid sequence of SEQ ID NO:2697.

In some embodiments, the antibody or antigen-binding fragment comprises a VH sequence that has at least 85% sequence identity to any one of SEQ ID NOS:2690, 2698, 2703, 2708, 2709, 2712, 2714, and 2752. In some embodiments, the VH sequence has at least 90% sequence identity to SEQ ID NO:2703. In some embodiments, the VH sequence has at least 95% sequence identity to SEQ ID NO:2703. In some embodiments, the VH sequence comprises SEQ ID NO:2703. In some embodiments, the VH sequence has at least 90% sequence identity to SEQ ID NO:2712. In some embodiments, the VH sequence has at least 95% sequence identity to SEQ ID NO:2712. In some embodiments, the VH sequence comprises SEQ ID NO:2712. In some embodiments, the VH sequence has at least 90% sequence identity to SEQ ID NO:79. In some embodiments, the VH sequence has at least 95% sequence identity to SEQ ID NO:79. In some embodiments, the VH sequence comprises SEQ ID NO:79.

In some embodiments, the antibody or antigen-binding fragment comprises a VL sequence that has at least 85% sequence identity to any one of SEQ ID NOS:2691, 2699, 2704, 2708, 2710, and 2741. In some embodiments, the VL sequence has at least 90% sequence identity to SEQ ID NO:2704. In some embodiments, the VL sequence has at least 95% sequence identity to SEQ ID NO:2704. In some embodiments, the VL sequence comprises SEQ ID NO:2704. In some embodiments, the VL sequence has at least 90% sequence identity to SEQ ID NO:2710. In some embodiments, the VL sequence has at least 95% sequence identity to SEQ ID NO:2710. In some embodiments, the VL sequence comprises SEQ ID NO:2710. In some embodiments, the VL sequence has at least 90% sequence identity to SEQ ID NO:2741. In some embodiments, the VL sequence has at least 95% sequence identity to SEQ ID NO:2741. In some embodiments, the VL sequence comprises SEQ ID NO:2741.

In some embodiments, the antibody or antigen-binding fragment comprises:
(a) a VH sequence comprising SEQ ID NO:2703 and a VL sequence comprising SEQ ID NO:2704; or
(b) a VH sequence comprising SEQ ID NO:2707 and a VL sequence comprising SEQ ID NO:2708; or
(c) a VH sequence comprising SEQ ID NO:2709 and a VL sequence comprising SEQ ID NO:2708; or
(d) a VH sequence comprising SEQ ID NO:2707 and a VL sequence comprising SEQ ID NO:2710; or
(e) a VH sequence comprising SEQ ID NO:79 and a VL sequence comprising SEQ ID NO:2710; or
(f) a VH sequence comprising SEQ ID NO:2712 and a VL sequence comprising SEQ ID NO:2708; or
(g) a VH sequence comprising SEQ ID NO:2714 and a VL sequence comprising SEQ ID NO:2708; or
(h) a VH sequence comprising SEQ ID NO:2712 and a VL sequence comprising SEQ ID NO:2710; or
(i) a VH sequence comprising SEQ ID NO:2714 and a VL sequence comprising SEQ ID NO:2710; or
(j) a VH sequence comprising SEQ ID NO:2690 and a VL sequence comprising SEQ ID NO:2691; or
(k) a VH sequence comprising SEQ ID NO:2698 and a VL sequence comprising SEQ ID NO:2699; or
(1) a VH sequence comprising SEQ ID NO:2712 and a VL sequence comprising SEQ ID NO:2741.

In some embodiments, an antibody or antigen-binding fragment thereof that specifically binds to TREM2 comprises:
(a) a CDR-H1 sequence comprising the amino acid sequence of any one of SEQ ID NOS:2692, 2700, and 2717;
(b) a CDR-H2 sequence comprising the amino acid sequence of any one of SEQ ID NOS:2693, 2701, 2713, 2718, 2727, 2729, and 2731;
(c) a CDR-H3 sequence comprising the amino acid sequence of any one of SEQ ID NOS:2694, 2702, 2705, and 2719;
(d) a CDR-L1 sequence comprising the amino acid sequence of any one of SEQ ID NOS:2695, 2711, and 2720;
(e) a CDR-L2 sequence comprising the amino acid sequence of any one of SEQ ID NOS:2696 and 2721; and
(f) a CDR-L3 sequence comprising the amino acid sequence of any one of SEQ ID NOS:2697, 2706, and 2722.

In some embodiments, the antibody or antigen-binding fragment comprises:
(a) a CDR-H1 comprising the amino acid sequence of SEQ ID NO:2692, a CDR-H2 comprising the amino acid sequence of SEQ ID NO:2693, a CDR-H3 comprising the amino acid sequence of SEQ ID NO:2694, a CDR-L1 comprising the amino acid sequence of SEQ ID NO:2695, a CDR-L2 comprising the amino acid sequence of SEQ ID NO:2696, and a CDR-L3 comprising the amino acid sequence of SEQ ID NO:2697; or
(b) a CDR-H1 comprising the amino acid sequence of SEQ ID NO:2692, a CDR-H2 comprising the amino acid sequence of SEQ ID NO:2693, a CDR-H3 comprising the amino acid sequence of SEQ ID NO:2705, a CDR-L1 comprising the amino acid sequence of SEQ ID NO:2695, a CDR-L2 comprising the amino acid sequence of SEQ ID NO:2696, and a CDR-L3 comprising the amino acid sequence of SEQ ID NO:2706; or
(c) a CDR-H1 comprising the amino acid sequence of SEQ ID NO:2692, a CDR-H2 comprising the amino acid sequence of SEQ ID NO:2693, a CDR-H3 comprising the amino acid sequence of SEQ ID NO:2705, a CDR-L1 comprising the amino acid sequence of SEQ ID NO:2711, a CDR-L2 comprising the amino acid sequence of SEQ ID NO:2696, and a CDR-L3 comprising the amino acid sequence of SEQ ID NO:2706; or
(d) a CDR-H1 comprising the amino acid sequence of SEQ ID NO:2692, a CDR-H2 comprising the amino acid sequence of SEQ ID NO:2713, a CDR-H3 comprising the amino acid sequence of SEQ ID NO:2705, a CDR-L1 comprising the amino acid sequence of SEQ ID NO:2695, a CDR-L2 comprising the amino acid sequence of SEQ ID NO:2696, and a CDR-L3 comprising the amino acid sequence of SEQ ID NO:2706; or
(e) a CDR-H1 comprising the amino acid sequence of SEQ ID NO:2692, a CDR-H2 comprising the amino acid sequence of SEQ ID NO:2713, a CDR-H3 comprising the amino acid sequence of SEQ ID NO:2705, a CDR-L1 comprising the amino acid sequence of SEQ ID NO:2711, a CDR-L2 comprising the amino acid sequence of SEQ ID NO:2696, and a CDR-L3 comprising the amino acid sequence of SEQ ID NO:2706; or
(f) a CDR-H1 comprising the amino acid sequence of SEQ ID NO:2700, a CDR-H2 comprising the amino acid sequence of SEQ ID NO:2701, a CDR-H3 comprising the amino acid sequence of SEQ ID NO:2702, a CDR-L1 comprising the amino acid sequence of SEQ ID NO:2695, a CDR-L2 comprising the amino acid sequence of SEQ ID NO:2696, and a CDR-L3 comprising the amino acid sequence of SEQ ID NO:2697;
(g) a CDR-H1 comprising the amino acid sequence of SEQ ID NO:2717, a CDR-H2 comprising the amino acid sequence of SEQ ID NO:2718, a CDR-H3 comprising the amino acid sequence of SEQ ID NO:2719, a CDR-L1 comprising the amino acid sequence of SEQ ID NO:2720, a CDR-L2 comprising the amino acid sequence of SEQ ID NO:2721, and a CDR-L3 comprising the amino acid sequence of SEQ ID NO:2722; or
(h) a CDR-H1 comprising the amino acid sequence of SEQ ID NO:2717, a CDR-H2 comprising the amino acid sequence of SEQ ID NO:2727, a CDR-H3 comprising the amino acid sequence of SEQ ID NO:2719, a CDR-L1 comprising the amino acid sequence of SEQ ID NO:2720, a CDR-L2 comprising the amino acid sequence of SEQ ID NO:2721, and a CDR-L3 comprising the amino acid sequence of SEQ ID NO:2722; or
(i) a CDR-H1 comprising the amino acid sequence of SEQ ID NO:2717, a CDR-H2 comprising the amino acid sequence of SEQ ID NO:2729, a CDR-H3 comprising the amino acid sequence of SEQ ID NO:2719, a CDR-L1 comprising the amino acid sequence of SEQ ID NO:2720, a CDR-L2 comprising the amino acid sequence of SEQ ID NO:2721, and a CDR-L3 comprising the amino acid sequence of SEQ ID NO:2722; or
(j) a CDR-H1 comprising the amino acid sequence of SEQ ID NO:2717, a CDR-H2 comprising the amino acid sequence of SEQ ID NO:2731, a CDR-H3 comprising the amino acid sequence of SEQ ID NO:2719, a CDR-L1 comprising the amino acid sequence of SEQ ID NO:2720, a CDR-L2 comprising the amino acid sequence of SEQ ID NO:2721, and a CDR-L3 comprising the amino acid sequence of SEQ ID NO:2722; or
(k) a CDR-H1 comprising the amino acid sequence of SEQ ID NO:2692, a CDR-H2 comprising the amino acid sequence of SEQ ID NO:2713, a CDR-H3 comprising the amino acid sequence of SEQ ID NO:2705, a CDR-L1 comprising the amino acid sequence of SEQ ID NO:2695, a CDR-L2 comprising the amino acid sequence of SEQ ID NO:2696, and a CDR-L3 comprising the amino acid sequence of SEQ ID NO:2697.

In some embodiments, the antibody or antigen-binding fragment comprises:
(a) a VH sequence that has at least 85% sequence identity to SEQ ID NO:2690 and a VL sequence that has at least 85% sequence identity to SEQ ID NO:2691; or
(b) a VH sequence that has at least 85% sequence identity to SEQ ID NO: 2698 and a VL sequence that has at least 85% sequence identity to SEQ ID NO:2699; or
(c) a VH sequence that has at least 85% sequence identity to SEQ ID NO:2703 and a VL sequence that has at least 85% sequence identity to SEQ ID NO:2704; or
(d) a VH sequence that has at least 85% sequence identity to SEQ ID NO:2707 and a VL sequence that has at least 85% sequence identity to SEQ ID NO:2708; or a VH sequence that has at least 85% sequence identity to SEQ ID NO:2709 and a VL sequence that has at least 85% sequence identity to SEQ ID NO:2708; or
(f) a VH sequence that has at least 85% sequence identity to SEQ ID NO: 2707 and a VL sequence that has at least 85% sequence identity to SEQ ID NO:2710; or
(g) a VH sequence that has at least 85% sequence identity to SEQ ID NO:79 and a VL sequence that has at least 85% sequence identity to SEQ ID NO:2710; or
(h) a VH sequence that has at least 85% sequence identity to SEQ ID NO:2712 and a VL sequence that has at least 85% sequence identity to SEQ ID NO:2708; or
(i) a VH sequence that has at least 85% sequence identity to SEQ ID NO:2714 and a VL sequence that has at least 85% sequence identity to SEQ ID NO:2708; or
(j) a VH sequence that has at least 85% sequence identity to SEQ ID NO:2712 and a VL sequence that has at least 85% sequence identity to SEQ ID NO:2710; or
(k) a VH sequence that has at least 85% sequence identity to SEQ ID NO: 2714 and a VL sequence that has at least 85% sequence identity to SEQ ID NO:2710; or
(l) a VH sequence that has at least 85% sequence identity to SEQ ID NO: 2715 and a VL sequence that has at least 85% sequence identity to SEQ ID NO:2716; or
(m) a VH sequence that has at least 85% sequence identity to SEQ ID NO:2723 and a VL sequence that has at least 85% sequence identity to SEQ ID NO:2724; or
(n) a VH sequence that has at least 85% sequence identity to SEQ ID NO: 2725 and a VL sequence that has at least 85% sequence identity to SEQ ID NO:2724; or
(o) a VH sequence that has at least 85% sequence identity to SEQ ID NO: 2726 and a VL sequence that has at least 85% sequence identity to SEQ ID NO:2724; or
(p) a VH sequence that has at least 85% sequence identity to SEQ ID NO:2728 and a VL sequence that has at least 85% sequence identity to SEQ ID NO:2724; or
(q) a VH sequence that has at least 85% sequence identity to SEQ ID NO:2730 and a VL sequence that has at least 85% sequence identity to SEQ ID NO:2724; or
(r) a VH sequence that has at least 85% sequence identity to SEQ ID NO: 2732 and a VL sequence that has at least 85% sequence identity to SEQ ID NO:2724; or
(s) a VH sequence that has at least 85% sequence identity to SEQ ID NO: 2733 and a VL sequence that has at least 85% sequence identity to SEQ ID NO:2724; or
(t) a VH sequence that has at least 85% sequence identity to SEQ ID NO: 2734 and a VL sequence that has at least 85% sequence identity to SEQ ID NO:2724; or
(u) a VH sequence that has at least 85% sequence identity to SEQ ID NO:2712 and a VL sequence that has at least 85% sequence identity to SEQ ID NO:2741.

In some embodiments, an antibody or antigen-binding fragment thereof that specifically binds to TREM2 recognizes an epitope that is the same or substantially the same as the epitope recognized by antibody clone selected from the group consisting of: CL0020306, Clone CL0020188, Clone CL0020188-1, Clone CL0020188-2, Clone CL0020188-3, Clone CL0020188-4, Clone CL0020188-5, Clone CL0020188-6, Clone CL0020188-7, Clone CL0020188-8, Clone CL0020307, Clone CL0020123, Clone CL0020123-1, Clone CL0020123-2, Clone CL0020123-3, Clone CL0020123-4, Clone CL0020123-5, Clone CL0020123-6, Clone CL0020123-7, and Clone CL0020123-8.

In some embodiments, the antibody or antigen-binding fragment recognizes an epitope that is the same or substantially the same as the epitope recognized by an antibody clone selected from the group consisting of: Clone CL0020123, Clone CL0020123-1, Clone CL0020123-2, Clone CL0020123-3, Clone CL0020123-4, Clone CL0020123-5, Clone CL0020123-6, Clone CL0020123-7, and Clone CL0020123-8. In particular embodiments, the antibody or antigen-binding fragment recognizes one or more of the following epitopes in SEQ ID NO: 1: (i) amino acid residues 55-63 (GEKGPCQRV (SEQ ID NO:2743)), (ii) amino acids 96-107 (TLRNLQPHDAGL (SEQ ID NO:2744)), and (iii) amino acid residues 126-129 (VEVL (SEQ ID NO:2745)). In another aspect, the disclosure features an isolated antibody or antigen-binding fragment thereof that specifically binds to a human TREM2, wherein the antibody or antigen-binding fragment thereof recognizes an epitope comprising or consisting of one or more of the following epitopes in SEQ ID NO: 1: (i) amino acid residues 55-63 (GEKGPCQRV (SEQ ID NO:2743)), (ii) amino acids 96-107 (TLRNLQPHDAGL (SEQ ID NO:2744)), and (iii) amino acid residues 126-129 (VEVL (SEQ ID NO:2745)). In some embodiments, the antibody or antigen-binding fragment recognizes an epitope that is the same or substantially the same as the epitope recognized by an antibody clone selected from the group consisting of: Clone CL0020188, Clone CL0020188-1, Clone CL0020188-2, Clone CL0020188-3, Clone CL0020188-4, Clone CL0020188-5, Clone CL0020188-6, Clone CL0020188-7, Clone CL0020188-8, Clone CL0020307, and Clone CL0020306. In particular embodiments, the antibody or antigen-binding fragment recognizes amino acid residues 143149 (FPGESES (SEQ ID NO:2742)) in SEQ ID NO: 1. In another aspect, the disclosure features an isolated antibody or antigen-binding fragment thereof that specifically binds to a human TREM2, wherein the antibody or antigen-binding fragment thereof recognizes an epitope comprising or consisting of amino acid residues 143-149 (FPGESES (SEQ ID NO:2742)) in SEQ ID NO:1.

In some embodiments, an antibody or antigen-binding fragment as disclosed herein decreases levels of soluble TREM2 protein (sTREM2). In some embodiments, an antibody or antigen-binding fragment as disclosed herein binds soluble TREM2 protein (sTREM2) in healthy human CSF or cynomolgus CSF with better potency compared to a reference antibody. In some embodiments, the reference antibody is represented by a combination of sequences selected from the group consisting of: SEQ ID NOS:2746 and 2747; SEQ ID NOS:2748 and 2749; and SEQ ID NOS:2750 and 2751.

In some embodiments, the antibody is an antibody having a VL, VH, full heavy chain sequence, full light chain sequence, a CDR sequence, or a full sequence disclosed in the “Informal Sequence Listing” Table IX of PCT Patent Application Publication No. WO 2020/172450 A1, which are reproduced below as TABLE L1.

TABLE L1

| Description | Sequence |
|---|---|
| Human TREM2 Protein | MEPLRLLILLFVTELSGAHNTTVFQGVAGQSLQVS |
| | CPYDSMKHWGRRKAWCRQLGEKGPCQRVVSTH |
| | NLWLLSFLRRWNGSTAITDDTLGGTLTITLRNLQP |
| | HDAGLYQCQSLHGSEADTLRKVLVEVLADPLDH |
| | RDAGDLWFPGESESFEDAHVEHSISRSLLEGEIPFP |
| | PTSILLLLACIFLIKILAASALWAAAWHGQKPGTH |
| | PPSELDCGHDPGYQLQTLPGLRDT (SEQ ID NO: 1) |
| CL0020306 VH | EVKLLDSGGGLVQAGGSLRLSCAGSGFTFTDFYM |
| | SWIRQPPGKAPEWLGVIRNKANGYTAGYNPSVK |
| | GRFTISRDNTQNILYLQMNTL |
| | RAEDTAIYYCARLSYGFDYWGQGVMVTVSS |
| | (SEQ ID NO: 2690) |
| CL0020306 VL | DIVMTQGALPNPVPSGESASITCQSSKSLLHSNGK |
| | TYLNWYLQRPGQSPQLLIYWMSTRASGVSDRFSG |
| | SGSGTDFTLKISSVEAEDVG |
| | VYYCQQFLEFPFTFGSGTKLEIK (SEQ ID NO :2691) |
| CL0020306 CDR-H1; CDR-H1 for | GFTFTDFYMS (SEQ ID NO: 2692) |
| CL0020188 and variants CL0020188-1, | |
| CL0020188-2, CL0020188-3, CL0020188-4, | |
| CL0020188-5, CL0020188-6, | |
| CL0020188-7, and CL0020188-8 | |
| CL0020306 CDR-H2; CDR-H2 for | VIRNKANGYTAGYNPSVKG (SEQ ID NO: 2693) |
| CL0020188 and variants CL0020188-1, | |
| CL0020188-2, CL0020188-3, and | |
| CL0020188-4 | |
| CL0020306 CDR-H3 | ARLSYGFDY (SEQ ID NO: 2694) |
| CL0020306 CDR-L1; CL0020307 CDR- | QSSKSLLHSNGKTYLN (SEQ ID NO: 2695) |
| L1; CL0020307-1 CDR-L1; CDR-L1 for | |
| CL0020188 and variants CL0020188-1, | |
| CL0020188-2, CL0020188-5, and | |
| CL0020188-6 | |
| CL0020306 CDR-L2; CL0020307 CDR-L2; | WMSTRAS (SEQ ID NO: 2696) |
| CL0020307-1 CDR-L2; CDR-L2 for | |
| CL0020188 and variants CL0020188-1, | |
| CL0020188-2, CL0020188-3, CL0020188-4, | |
| CL0020188-5, CL0020188-6, CL0020188-7, | |
| and CL0020188-8 | |
| CL0020306 CDR-L3; CL0020307 CDR- | QQFLEFPFT (SEQ ID NO: 2697) |
| L3; CL0020307-1 CDR-L3 | |
| CL0020307 V_{H} | EVKLLESGGGLVQPGGSLRLSCAASGFTFTNFYM |
| | SWIRQPPGRAPEWLGVIRNRPNGYTTDYNPSVKG |
| | RFTISRDNTQNILYLQMSTLRADDTAFYYCTRLTY |
| | GFDYWGQGVMVTVSS (SEQ ID NO: 2698) |
| CL0020307 VL | DIVMTQGALPNPVPSGESASITCQSSKSLLHSNGK |
| | TYLNWYLQRPGQSPQLLIYWMSTRASGVSDRFSG |
| | SGSGTDFTLKIS |
| | SVEAEVVGVYYCQQFLEFPFTFGSGTKLEIK |
| | (SEQ ID NO: 2699) |
| CL0020307 CDR-H1 | GFTFTNFYMS (SEQ ID NO: 2700) |
| CL0020307 CDR-H2 | VIRNRPNGYTTDYNPSVKG (SEQ ID NO: 2701) |
| CL0020307 CDR-H3 | TRLTYGFDY (SEQ ID NO: 2702) |
| CL0020188 VH | EVKLLDSGGGLVQAGGSLRLSCAGSGFTFTDFYM |
| | SWIRQPPGKAPEWLGVIRNKANGYTAGYNPSVK |
| | GRFTISRDNTQNILYLQMNTLRAEDTAIYYCARLT |
| | YGFDYWGQGVMVTVSS (SEQ ID NO: 2703) |
| CL0020188 VL | DIVMTQGALPNPVPSGESASITCQSSKSLLHSNGK |
| | TYLNWYLQR |
| | PGQSPQLLIYWMSTRASGVSDRFSGSGSGTDFTLK |
| | ISSVEAEDVGVYYCQQFLEYPFTFGSGTKLEIK |
| | (SEQ ID NO: 2704) |
| CDR-H3 for CL0020188 and variants | ARLTYGFDY (SEQ ID NO: 2705) |
| CL0020188-1, CL0020188-2, CL0020188-3, | |
| CL0020188-4, CL0020188-5, | |
| CL0020188-6, CL0020188-7, and | |
| CL0020188-8 | |
| CDR-L3 for CL0020188 and variants | QQFLEYPFT (SEQ ID NO: 2706) |
| CL0020188-1, CL0020188-2, CL0020188- | |
| 3, CL0020188-4, CL0020188-5, | |
| CL0020188-6, CL0020188-7, and | |
| CL0020188-8 | |
| CL0020188-1 VH; CL0020188-3 VH | EVQLVESGGGLVQPGGSLRLSCAASGFTFTDFYM |
| | SWVRQAPGKGLEWVSVIRNKANGYTAGYNPSVK |
| | GRFTISRDNSKNTLYLQMNSLRAEDTAVYYCARL |
| | TYGFDYWGQGTLVTVSS (SEQ ID NO: 2707) |
| CL0020188-1 VL; CL0020188-2 VL; | DIVMTQTPLSLPVTPGEPASISCQSSKSLLHSNGKT |
| CL0020188-5 VL; CL0020188-6 VL | YLNWYLQKPGQSPQLLIYWMSTRASGVPDRFSGS |
| | GSGTDFTLKISRVEAEDVGVYYCQQFLEYPFTFG |
| | QGTKVEIK (SEQ ID NO: 2708) |
| CL0020188-2 VH | EVQLVESGGGLVQPGGSLRLSCAGSGFTFTDFYM |
| | SWVRQAPGKGLEWVSVIRNKANGYTAGYNPSVK |
| | GRFTISRDNSKNTLYLQMNSLRAEDTAVYYCARL |
| | TYGFDYWGQGTLVTVSS (SEQ ID NO: 2709) |
| CL0020188-3 VL; CL0020188-4 VL; | DIVMTQTPLSLPVTPGEPASISCQSSKSLLHSTGKT |
| CL0020188-7 VL; CL0020188-8 VL | YLNWYLQKPGQSPQLLIYWMSTRASGVPDRFSGS |
| | GSGTDFTLKISRVEAEDVGVYYCQQFLEYPFTFG |
| | QGTKVEIK (SEQ ID NO: 2710) |
| CDR-L1 for variants CL0020188-3, | QSSKSLLHSTGKTYLN (SEQ ID NO: 2711) |
| CL0020188-4, CL0020188-7, and | |
| CL0020188-8 | |
| CL0020188-5 VH; CL0020188-7 VH | EVQLVESGGGLVQPGGSLRLSCAASGFTFTDFYM |
| | SWVRQAPGKGLEWVSVIRNKANAYTAGYNPSVK |
| | GRFTISRDNSKNTLYLQMNSLRAEDTAVYYCARL |
| | TYGFDYWGQGTLVTVSS (SEQ ID NO: 2712) |
| CDR-H2 for variants CL0020188-5, | VIRNKANAYTAGYNPSVKG (SEQ ID NO: 2713) |
| CL0020188-6, CL0020188-7, and | |
| CL0020188-8 | |
| CL0020188-6 VH; CL0020188-8 VH | EVQLVESGGGLVQPGGSLRLSCAGSGFTFTDFYM |
| | SWVRQAPGKGPEWLSVIRNKANAYTAGYNPSVK |
| | GRFTISRDNSKNTLYLQMNSLRAEDTAVYYCARL |
| | TYGFDYWGQGTLVTVSS (SEQ ID NO: 2714) |
| CL0020123 VH | EVQLQQSGAELVRSGASVKLSCTASGFSIEDFYIH |
| | WVKQRPEQGLEWIGWIDPENGDSKYAPKFQGKA |
| | TMTADTSSNTAYLHLSSLTSEDTAVYYCHADHGN |
| | YGSTMDYWGQGTSVTVSS (SEQ ID NO: 2715) |
| CL0020123 VL | DIQMNQSPSSLSASLGDTVTITCHASQHINVWLSW |
| | YQQKPGDHPKLLIYKASNLHTGVPSRFSGSGSGT |
| | GFTLTISSLQPEDIATYYCQQGQTYPRTFGGGTKL |
| | EIK (SEQ ID NO: 2716) |
| CDR-H1 for CL0020123 and variants | GFSIEDFYIH (SEQ ID NO: 2717) |
| CL0020123-1, CL0020123-2, CL0020123- | |
| 3, CL0020123-4, CL0020123-5, | |
| CL0020123-6, CL0020123-7, and | |
| CL0020123-8 | |
| CDR-H2 for CL0020123 and variants | WIDPENGDSKYAPKFQG (SEQ ID NO: 2718) |
| CL0020123-1 and CL0020123-2 | |
| CDR-H3 for CL0020123 and variants | HADHGNYGSTMDY (SEQ ID NO: 2719) |
| CL0020123-1, CL0020123-2, CL0020123- | |
| 3, CL0020123-4, CL0020123-5, | |
| CL0020123-6, CL0020123-7, and | |
| CL0020123-8 | |
| CDR-L1 for CL0020123 and variants | HASQHINVWLS (SEQ ID NO: 2720) |
| CL0020123-1, CL0020123-2, CL0020123- | |
| 3, CL0020123-4, CL0020123-5, | |
| CL0020123-6, CL0020123-7, and | |
| CL0020123-8 | |
| CDR-L2 for CL0020123 and variants | KASNLHT (SEQ ID NO: 2721) |
| CL0020123-1, CL0020123-2, CL0020123-3, | |
| CL0020123-4, CL0020123-5, | |
| CL0020123-6, CL0020123-7, and | |
| CL0020123-8 | |
| CDR-L3 for CL0020123 and variants | QQGQTYPRT (SEQ ID NO: 2722) |
| CL0020123-1, CL0020123-2, CL0020123-3, | |
| CL0020123-4, CL0020123-5, | |
| CL0020123-6, CL0020123-7, and | |
| CL0020123-8 | |
| CL0020123-1 VH | QVQLVQSGAEVKKPGASVKVSCKASGFSIEDFYI |
| | HWVRQAPGQGLEWMGWIDPENGDSKYAPKFQG |
| | RATITADTSTSTAYMELSSLRSEDTAVYYCHADH |
| | GNYGSTMDYWGQGTLVTVSS (SEQ ID NO: 2723) |
| CL0020123-1 VL; CL0020123-2 VL; | DIQMTQSPSSLSASVGDRVTITCHASQHINVWLS |
| CL0020123-3 VL; CL0020123-4 VL | WYQQKPGKAPKLLIYKASNLHTGVPSRFSGSGSG |
| CL0020123-5 VL; CL0020123-6 VL; | TDFTLTISSLQPEDFATYYCQQGQTYPRTFGQGTK |
| CL0020123-7 VL; CL0020123-8 VL | VEIK (SEQ ID NO: 2724) |
| CL0020123-2 VH | QVQLVQSGAEVKKPGASVKVSCKASGFSIEDFYI |
| | HWVRQAPGQGLEWMGWIDPENGDSKYAPKFQG |
| | RVTITADTSTSTAYMELSSLRSEDTAVYYCHADH |
| | GNYGSTMDYWGQGTLVTVSS (SEQ ID NO: 2725) |
| CL0020123-3 VH | QVQLVQSGAEVKKPGASVKVSCKASGFSIEDFYI |
| | HWVRQAPGQGLEWMGWIDPEQGDSKYAPKFQG |
| | RATITADTSTSTAYMELSSLRSEDTAVYYCHADH |
| | GNYGSTMDYWGQGTLVTVSS (SEQ ID NO: 2726) |
| CDR-H2 for variants CL0020123-3 and | WIDPEQGDSKYAPKFQG (SEQ ID NO: 2727) |
| CL0020123-6 | |
| CL0020123-4 VH | QVQLVQSGAEVKKPGASVKVSCKASGFSIEDFYI |
| | HWVRQAPGQGLEWMGWIDPENGESKYAPKFQG |
| | RATITADTSTSTAYMELSSLRSEDTAVYYCHADH |
| | GNYGSTMDYWGQGTLVTVSS (SEQ ID NO: 2728) |
| CDR-H2 for variants CL0020123-4 and | WIDPENGESKYAPKFQG (SEQ ID NO: 2729) |
| CL0020123-7 | |
| CL0020123-5 VH | QVQLVQSGAEVKKPGASVKVSCKASGFSIEDFYI |
| | HWVRQAPGQGLEWMGWIDPEQGESKYAPKFQG |
| | RATITADTSTSTAYMELSSLRSEDTAVYYCHADH |
| | GNYGSTMDYWGQGTLVTVSS (SEQ ID NO: 2730) |
| CDR-H2 for variants CL0020123-5 and | WIDPEQGESKYAPKFQG (SEQ ID NO: 2731) |
| CL0020123-8 | |
| CL0020123-6 VH | QVQLVQSGAEVKKPGASVKVSCKASGFSIEDFYI |
| | HWVRQAPGQGLEWMGWIDPEQGDSKYAPKFQG |
| | RVTITADTSTSTAYMELSSLRSEDTAVYYCHADH |
| | GNYGSTMDYWGQGTLVTVSS (SEQ ID NO: 2732) |
| CL0020123-7 VH | QVQLVQSGAEVKKPGASVKVSCKASGFSIEDFYI |
| | HWVRQAPGQGLEWMGWIDPENGESKYAPKFQG |
| | RVTITADTSTSTAYMELSSLRSEDTAVYYCHADH |
| | GNYGSTMDYWGQGTLVTVSS (SEQ ID NO: 2733) |
| CL0020123-8 VH | QVQLVQSGAEVKKPGASVKVSCKASGFSIEDFYI |
| | HWVRQAPGQGLEWMGWIDPEQGESKYAPKFQG |
| | RVTITADTSTSTAYMELSSLRSEDTAVYYCHADH |
| | GNYGSTMDYWGQGTLVTVSS (SEQ ID NO: 2734) |
| CDR-H2 consensus sequence | W-I-D-P-E-β6-G-β8-S-K-Y-A-P-K-F-Q-G, wherein (β6 |
| | is N or Q and (β8 is D or E (SEQ ID NO: 2735) |
| CDR-H1 consensus sequence | G-F-T-F-T-α6-F-Y-M-S, wherein α6 is D or N |
| | (SEQ ID NO: 2736) |
| CDR-H2 consensus sequence | V-I-R-N-β5-β6-N-β8-Y-T-β11-β12-Y-N-P-S-V-K-G, |
| | wherein β5 is K or R; β6 is A or P; β8 is G or A; |
| | β11 is A or T; and β12 is G or D (SEQ ID NO: 2737) |
| CDR-H3 consensus sequence | γ1-R-L-γ4-Y-G-F-D-Y, wherein γ1 is A or T; and γ4 |
| | is T or S (SEQ ID NO: 2738) |
| CDR-L1 consensus sequence | Q-S-S-K-S-L-L-H-S-δ10-G-K-T-Y-L-N, wherein δ10 is |
| | N or T (SEQ ID NO: 2739) |
| CDR-L3 consensus sequence | Q-Q-F-L-E-f6-P-F-T, wherein f6 is Y or F |
| | (SEQ ID NO: 2740) |
| CL0020307-1 VL | DIVMTQSPDSLAVSLGERATINCQSSKSLLHSNGK |
| | TYLNWYQQKPGQPPKLLIYWMSTRASGVPDRFS |
| | GSGSGTDFTLTISSLQAEDVAVYYCQQFLEFPFTF |
| | GQGTKVEIK (SEQ ID NO: 2741) |
| TREM2 epitope | FPGESES (SEQ ID NO: 2742) |
| TREM2 epitope | GEKGPCQRV (SEQ ID NO: 2743) |
| TREM2 epitope | TLRNLQPHDAGL (SEQ ID NO: 2744) |
| TREM2 epitope | VEVL (SEQ ID NO: 2745) |
| Reference antibody #1 VL | DIQMTQSPSSVSASVGDRVTITCRASQGISNWLA |
| | WYQQKPGKAPKLLIYAASSLQVGVPLRFSGSGSG |
| | TDFTLTISSLQPEDFATYYCQQADSFPRNFGQGTK |
| | LEIK (SEQ ID NO: 2746) |
| Reference antibody #1 VH | EVQLVQSGAEVKKPGESLKISCKGSGHSFTNYWI |
| | AWVRQMPGKGLEWMGIIYPGDSDTRYSPSFQGQ |
| | VTISADKSISTAYLQWSSLKASDTAVYFCARQRTF |
| | YYDSSGYFDYWGQGTLVTVSS (SEQ ID NO: 2747) |
| Reference antibody #2 VL | DIQMTQSPSSVSASVGDRVTITCRASQGISSWLAW |
| | YQQKPGKAPKLLIYAASSLQNGVPSRFSGSGSGT |
| | DFTLTISSLQPEDFATYFCQQADSFPRTFGQGTKL |
| | EIK (SEQ ID NO: 2748) |
| Reference antibody #2 VH | EVQLVQSGAEVKKPGESLKISCKGSGYSFTSYWIA |
| | WVRQMPGKGLEWMGIIYPGDSDTRYSPSFQGQV |
| | TISADKSISTAYLQWSSLKASDTAMYFCARQRTF |
| | YYDSSDYFDYWGQGTLVTVSS (SEQ ID NO: 2749) |
| Reference antibody #3 VL | DVVMTQSPDSLAVSLGERATINCRSSQSLVHSNR |
| | YTYLHWYQQKPGQSPKWYKVSNRFSGVPDRFS |
| | GSGSGTDFTLKISRVEAEDVGVYYCSQSTRVPYTF |
| | GQGTKLEIK (SEQ ID NO: 2750) |
| Reference antibody #3 VH | QVQLVQSGAEVKKPGASVKVSCKASGYAFSSQW |
| | MNWVRQAPGQRLEWIGRIYPGGGDTNYAGKFQG |
| | RVTITADTSASTAYMELSSLRSEDTAVYYCARLL |
| | RNQPGESYAMDYWGQGTLVTVSS |
| | (SEQ ID NO: 2751) |
| CL0020188-4 VH | EVQLVESGGGLVQPGGSLRLSCAGSGFTFTDFYM |
| | SWVRQAPGKGPEWLSVIRNKANGYTAGYNPSVK |
| | GRFTISRDNSKNTLYLQMNSLRAEDTAVYYCARL |
| | TYGFDYWGQGTLVTVSS (SEQ ID NO: 2752) |

In some embodiments, each of the light chain variable regions and each of the heavy chain variable regions disclosed in TABLE L1 as well as specific combinations thereof and other embodiments of the anti-TREM2 antibody described in the '450 application and herein may be attached to the light chain constant regions (TABLE EN1) and heavy chain constant regions (TABLE EN2) to form complete antibody light and heavy chains, respectively, as further discussed below. Further, each of the generated heavy and light chain sequences may be combined to form a complete antibody structure. It should be understood that the heavy chain and light chain variable regions provided herein can also be attached to other constant domains having different sequences than the exemplary sequences listed herein.

M. PCT Patent Application Publication No. WO2021/101823A1

In some embodiments, the TREM2 agonist is an antibody, or an antigen-binding fragment thereof, as described in PCT Patent Application Publication No. WO2021101823A1 (“the '823 application”), which is incorporated by reference herein, in its entirety.

In some embodiments, the antibody or antigen-binding fragment thereof comprises:
(a) a CDR-H1 sequence comprising the sequence of GFSFNTYWIG (SEQ ID NO:2753);
(b) a CDR-H2 sequence comprising the sequence of IIYPGDQDIRYSPSFQG (SEQ ID NO:2754;
(c) a CDR-H3 sequence comprising the sequence of ARYGRYIYGYGGYHGMDV (SEQ ID NO:2755;
(d) CDR-L1 sequence comprising the sequence of RASQAIRDDLG (SEQ ID NO:2756);
(e) a CDR-L2 sequence comprising the sequence of YAASSLQS (SEQ ID NO:2757); and
(f) a CDR-L3 sequence comprising the sequence of LQNYNYPHT (SEQ ID NO:2758).

In some embodiments, the antibody or antigen-binding fragment comprises a CDR-H1 comprising the amino acid sequence of SEQ ID NO:2753, a CDR-H2 comprising the amino acid sequence of SEQ ID NO:2754, a CDR-H3 comprising the amino acid sequence of SEQ ID NO:2755, a CDR-L1 comprising the amino acid sequence of SEQ ID NO:2756, a CDR-L2 comprising the amino acid sequence of SEQ ID NO:2757, and a CDR-L3 comprising the amino acid sequence of SEQ ID NO:2758.

In some embodiments, the antibody or antigen-binding fragment comprises a VH sequence that has at least 85% sequence identity to SEQ ID NO:2759. In some embodiments, the V_{H}sequence has at least 90% sequence identity to SEQ ID NO:2759. In some embodiments, the V_{H}sequence has at least 95% sequence identity to SEQ ID NO:2759. In some embodiments, the V_{H}sequence comprises SEQ ID NO:2759.

In some embodiments, the antibody or antigen-binding fragment comprises a V_{L} sequence that has at least 85% sequence identity to SEQ ID NO:2760. In some embodiments, the V_{L} sequence has at least 90% sequence identity to SEQ ID NO:2760. In some embodiments, the V_{L} sequence has at least 95% sequence identity to SEQ ID NO:2760. In some embodiments, the V_{L} sequence comprises SEQ ID NO:2760.

In some embodiments, the antibody or antigen-binding fragment comprises a VH sequence comprising SEQ ID NO:2759 and a V_{L} sequence comprising SEQ ID NO:2760.

In some embodiments, an antibody or antigen-binding fragment thereof that specifically binds to TREM2 recognizes an epitope that is the same or substantially the same as the epitope recognized by Antibody 1 of the '823 application.

In some embodiments, the antibody or antigen-binding fragment recognizes an epitope present on the extracellular domain of a human TREM2. In particular embodiments, the antibody or antigen-binding fragment recognizes an epitope present on the extracellular domain of a human TREM2 in SEQ ID NO:2763. In some embodiments, the antibody or antigen-binding fragment recognizes an epitope present on the extracellular domain of a mouse TREM2. In particular embodiments, the antibody or antigen-binding fragment recognizes an epitope present on the extracellular domain of a mouse TREM2 in SEQ ID NO:2764. In some embodiments, the antibody or antigen-binding fragment recognizes an epitope present on the extracellular domain of a rat TREM2. In particular embodiments, the antibody or antigen-binding fragment recognizes an epitope present on the extracellular domain of a rat TREM2 in SEQ ID NO:2765. In some embodiments, the antibody or antigen-binding fragment recognizes an epitope present on the extracellular domain of a rabbit TREM2. In particular embodiments, the antibody or antigen-binding fragment recognizes an epitope present on the extracellular domain of a rabbit TREM2 in SEQ ID NO:2766. In some embodiments, the antibody or antigen-binding fragment recognizes an epitope present on the extracellular domain of a cynomolgus monkey TREM2. In particular embodiments, the antibody or antigen-binding fragment recognizes an epitope present on the extracellular domain of a cynomolgus monkey TREM2 in SEQ ID NO:2767.

In some embodiments, the antibody is an antibody having a VL, VH, full heavy chain sequence, full light chain sequence, a CDR sequence, or a full sequence disclosed in the “SEQUENCE” Table of PCT Patent Application Publication No. WO2021101823A1, which are reproduced below as TABLE M1.

TABLE M1

| Description | Sequence |
|---|---|
| ′823 Antibody 1 CDR-H1 | GFSFNTYWIG (SEQ ID NO: 2753) |
| ′823 Antibody 1 CDR-H2 | IIYPGDQDIRYSPSFQG (SEQ ID NO: 2754) |
| ′823 Antibody 1 CDR-H3 | ARYGRYIYGYGGYHGMDV (SEQ ID NO: 2755) |
| ′823 Antibody 1 CDR-L1 | RASQAIRDDLG (SEQ ID NO: 2756) |
| ′823 Antibody 1 CDR-L2 | YAASSLQS (SEQ ID NO: 2757 |
| ′823 Antibody 1 CDR-L3 | LQNYNYPHT (SEQ ID NO: 2758) |
| ′823 Antibody 1 VH | EVQLVQSGAEVKKPGESLKISCKGSGFSFNTYWIGWVRQMPG |
| | KGLEWMGIIYPGDQDIRYSPSFQGQVTISADKSISTAYLQWSSL |
| | KASDTAMYYCARYGRYIYGYGGYHGMDVWGQGTTVTVSS |
| | (SEQ ID NO: 2759) |
| ′823 Antibody 1 VL | DIQMTQSPSSLSASVGDRVTITCRASQAIRDDLGWYQQKPGKA |
| | PKLLIYAASSLQSGVPSRFSGSGSGTDFTLTISSLQPEDFATYYC |
| | LQNYNYPHTFGQGTKLEIK (SEQ ID NO: 2760) |
| ′823 Antibody 1 Heavy | EVQLVQSGAEVKKPGESLKISCKGSGFSFNTYWIGWVRQMPG |
| Chain | KGLEWMGIIYPGDQDIRYSPSFQGQVTISADKSISTAYLQWSSL |
| | KASDTAMYYCARYGRYIYGYGGYHGMDVWGQGTTVTVSSA |
| | STKGPSVFPLAPCSRSTSESTAALGCLVKDYFPEPVTVSWNSG |
| | ALTSGVHTFPAVLQSSGLYSLSSVVTVPSSSLGTKTYTCNVDH |
| | KPSNTKVDKRVESKYGPPCPPCPAPEAAGGPSVFLFPPKPKDT |
| | LMISRTPEVTCVVVDVSQEDPEVQFNWYVDGVEVHNAKTKP |
| | REEQFNSTYRVVSVLTVLHQDWLNGKEYKCKVSNKGLPSSIE |
| | KTISKAKGQPREPQVYTLPP SQEEMTKNQVSLTCLVKGFYPSD |
| | IAVEWESNGQPENNYKTTPPVLDSDGSFFLYSRLTVDKSRWQE |
| | GNVFSCSVMHEALHNHYTQKSLSLSLG (SEQ ID NO: 2761) |
| ′823 Antibody 1 Light Chain | DIQMTQSPSSLSASVGDRVTITCRASQAIRDDLGWYQQKPGKA |
| | PKLLIYAASSLQSGVPSRFSGSGSGTDFTLTISSLQPEDFATYYC |
| | LQNYNYPHTFGQGTKLEIKRTVAAPSVFIFPPSDEQLKSGTASV |
| | VCLLNNFYPREAKVQWKVDNALQSGNSQESVTEQDSKDSTYS |
| | LSSTLTLSKADYEKHKVYACEVTHQGLSSPVTKSFNRGEC |
| | (SEQ ID NO: 2762) |
| Human TREM2 ECD-His | HNTTVFQGVAGQSLQVSCPYDSMKHWGRRKAWCRQLGEKG |
| | PCQRVVSTHNLWLLSFLRRWNGSTAITDDTLGGTLTITLRNLQ |
| | PHDAGLYQCQSLHGSEADTLRKVLVEVLADPLDHRDAGDLW |
| | FPGESESFEDAHVEHSISRSLLEGEIPFPPTSHHHHHH |
| | (SEQ ID NO: 2763) |
| Mouse TREM2 ECD-His | LNTTVLQGMAGQSLRVSCTYDALKHWGRRKAWCRQLGEEG |
| | PCQRVVSTHGVWLLAFLKKRNGSTVIADDTLAGTVTITLKNL |
| | QAGDAGLYQCQSLRGREAEVLQKVLVEVLEDPLDDQDAGDL |
| | WVPEESS SFEGAQVEHSTSRNQETSFPPTSHHHHHH |
| | (SEQ ID NO: 2764) |
| Rat TREM2 ECD-His | NTTVFQGVAGQSLRVSCPYDSATHWGRRKAWCRQLGEEGPC |
| | ERVVSTHSWWLLSFLKRRNGSTAITDDALGGTLTVTLRDLQA |
| | QDAGVYQCQSLQGREASTLQKILVEVLTEPLEHEHAGDFWVP |
| | EESGSFEDPPVERSSSRSPSEGEPSFPPASGGGGQHHHHHH |
| | (SEQ ID NO: 2765) |
| Rabbit TREM2 ECD-His | NTTVLQGVAGQSLRVSCTYDALRHWGRRKAWCRQLAEEGPC |
| | QRVVSTHGVWLLAFLRKQNGSTVITDDTLAGTVTITLRNLQA |
| | GDAGLYQCQSLRGREAEVLQKVVVEVLEDPLDDQDAGDLWV |
| | PEESESFEGAQVEHSTSRSQSGGGGQHHHHHH |
| | (SEQ ID NO: 2766) |
| Cynomolgus monkey | HNTTVFQGVEGQSLQVSCPYDSMKHWGRRKAWCRQLGEKGP |
| TREM2 ECD-His | CQRVVSTHNLWLLSFLRRRNGSTAITDDTLGGTLTITLRNLQPH |
| | DAGFYQCQSLHGSEADTLRKVLVEVLADPLDHRDAGDLWVP |
| | GESESFEDAHVEHSISRPSQGSHLPSCLSKEGGGGQHHHHHH |
| | (SEQ ID NO: 2767) |

In some embodiments, each of the light chain variable regions and each of the heavy chain variable regions disclosed in TABLE M1 as well as specific combinations thereof and other embodiments of the anti-TREM2 antibody described in the '823 application and herein may be attached to the light chain constant regions (TABLE EN1) and heavy chain constant regions (TABLE EN2) to form complete antibody light and heavy chains, respectively, as further discussed below. Further, each of the generated heavy and light chain sequences may be combined to form a complete antibody structure. It should be understood that the heavy chain and light chain variable regions provided herein can also be attached to other constant domains having different sequences than the exemplary sequences listed herein.

### Exemplary Anti-TREM2 Antibodies

In some embodiments, the TREM2 agonist antigen binding protein comprises a CDRL1 or a variant thereof having one, two, three or four amino acid substitutions; a CDRL2, or a variant thereof having one, two, three or four amino acid substitutions; a CDRL3, or a variant thereof having one, two, three or four amino acid substitutions; a CDRH1, or a variant thereof having one, two, three or four amino acid substitutions; a CDRH2, or a variant thereof having one, two, three or four amino acid substitutions; and a CDRH3, or a variant thereof having one, two, three or four amino acid substitutions, where the amino acid sequences of the CDRL1, CDRL2, CDRL3, CDRH1, CDRH2, and CDRH3 are provided in TABLES EX1 and EX2 below, along with exemplary light chain and variable regions.

TABLE EX1
Exemplary Anti-TREM2 Antibody Light Chain Variable Regions

| VL Amino Acid Sequence | CDRL1 | CDRL2 | CDRL3 |
|---|---|---|---|
| EIVMTQSPATLSVSPGERATLSCR | RASQSVSSNLA | GASTRAT | LQDNNFPPT |
| ASQSVSSNLAWFQQKPGQAPRLL | (SEQ ID NO: 10) | (SEQ ID NO: 23) | (SEQ ID NO: 372) |
| IYGASTRATGIPARFSGSGSGTEF | | | |
| TLTISSLQPEDFAVYYCLQDNNFP | | | |
| PTFGQGTKVDIK (SEQ ID NO: 330) | | | |

TABLE EX2
Exemplary Anti-TREM2 Antibody Heavy Chain Variable Regions

| VH Amino Acid Sequence | CDRH1 | CDRH2 | CDRH3 |
|---|---|---|---|
| EVQLVQSGAEVKKPGESLKIS | SWIG | IIYPGDADARYSPSF | RRQGIFGDALDF |
| CKGSGYSFTSYWIGWVRQMP | (SEQ ID NO: 81) | QG (SEQ ID NO: 373) | (SEQ ID NO: 374) |
| GKGLEWMGIIYPGDADARYS | | | |
| PSFQGQVTISADKSISTAYLQ | | | |
| WSSLKASDTAMYFCARRRQ | | | |
| GIFGDALDFWGQGTLVTVSS | | | |
| (SEQ ID NO: 331) | | | |

As noted above, anti-TREM2 antibodies may comprise one or more of the CDRs presented in TABLE EX1 (light chain CDRs; i.e. CDRLs) and TABLE EX2 (heavy chain CDRs, i.e. CDRHs).

In some embodiments, an anti-TREM2 antibody comprises a light chain comprising a CDRL1 having an amino acid sequence according to SEQ ID NO:10, a CDRL2 having an amino acid sequence according to SEQ ID NO:23, a CDRL3 having an amino acid sequence according to SEQ ID NO:372, or any CDRL1, CDRL2, or CDRL3 amino acid sequence that contains one or more, e.g., one, two, three, four or more amino acid substitutions (e.g., conservative amino acid substitutions), deletions or insertions of no more than five, four, three, two, or one amino acids to any of SEQ ID NOS: 10, 23, and 372. Such substitutions, deletions, and insertions would retain significant anti-TREM2 binding activity In these and other embodiments, an anti-TREM2 antibody comprises a CDRH1 having an amino acid sequence according to SEQ ID NO:81, a CDRH2 having an amino acid sequence according to SEQ ID NO:373, a CDRH3 having an amino acid sequence according to SEQ ID NO:374, or any CDRH1, CDRH2, or CDRH3 having an amino acid sequence that contains one or more, e.g., one, two, three, four or more amino acid substitutions (e.g., conservative amino acid substitutions), deletions or insertions of no more than five, four, three, two, or one amino acids to any of SEQ ID NOS:81, 373, and 374. Such substitutions, deletions, and insertions would retain significant anti-TREM2 binding activity

In some embodiments, an anti-TREM2 antibody comprises a light chain variable region comprising a CDRL1 having an amino acid sequence according to SEQ ID NO: 10; a CDRL2 having an amino acid sequence according to SEQ ID NO:23; and a CDRL3 having an amino acid sequence according to SEQ ID NO:372, and a heavy chain variable region comprising a CDRH1 having an amino acid sequence according to SEQ ID NO:81; a CDRH2 having an amino acid sequence according to SEQ ID NO:373; and a CDRH3 having an amino acid sequence according to SEQ ID NO:374.

In some embodiments, an anti-TREM2 antibody comprises a light chain variable region having an amino acid sequence according to SEQ ID NO:330, or any amino acid sequence that contains one or more, e.g., one, two, three, four or more amino acid substitutions (e.g., conservative amino acid substitutions), deletions or insertions of no more than five, four, three, two, or one amino acids to SEQ ID NO:330. Such substitutions, deletions, and insertions would retain significant anti-TREM2 binding activity. In some embodiments, an anti-TREM2 antibody comprises a heavy chain variable region having an amino acid sequence according to SEQ ID NO:331, or any amino acid sequence that contains one or more, e.g., one, two, three, four or more amino acid substitutions (e.g., conservative amino acid substitutions), deletions or insertions of no more than five, four, three, two, or one amino acids to SEQ ID NO:331. Such substitutions, deletions, and insertions would retain significant anti-TREM2 binding activity.

In specific embodiments, an anti-TREM2 antibody comprises a light chain variable region having an amino acid sequence according to SEQ ID NO:330, and a heavy chain variable region having an amino acid sequence according to SEQ ID NO:331.

In some embodiments, an anti-TREM2 antibody comprises a heavy chain amino acid sequence, and/or a light chain amino acid sequence selected from TABLE EX3. TABLE EX3 shows exemplary anti-TREM2 antibody heavy and light chains for exemplary antibodies “Ab-1”, “Ab-2”, and “Ab-3”.

TABLE EX3
Exemplary Anti-TREM2 Antibody Heavy and Light Chains

| Chain | Description | Amino Acid Sequence |
|---|---|---|
| Light | Ab-1 | EIVMTQSPATLSVSPGERATLSCRASQSVSSNLAWFQQKPGQAPRLLI |
| Chain | | YGASTRATGIPARFSGSGSGTEFTLTISSLQPEDFAVYYCLQDNNFPPT |
| | | FGQGTKVDIKRTVAAPSVFIFPPSDEQLKSGTASVVCLLNNFYPREAK |
| | | VQWKVDNALQSGNSQESVTEQDSKDSTYSLSSTLTLSKADYEKHKV |
| | | YACEVTHQGLSSPVTKSFNRGEC (SEQ ID NO: 2777) |
| | Ab-1 w/ | MDMRVPAQLLGLLLLWLRGARCEIVMTQSPATLSVSPGERATLSCR |
| | leader | ASQSVSSNLAWFQQKPGQAPRLLIYGASTRATGIPARFSGSGSGTEFT |
| | sequence | LTISSLQPEDFAVYYCLQDNNFPPTFGQGTKVDIKRTVAAPSVFIFPPS |
| | | DEQLKSGTASVVCLLNNFYPREAKVQWKVDNALQSGNSQESVTEQ |
| | | DSKDSTYSLSSTLTLSKADYEKHKVYACEVTHQGLSSPVTKSFNRGE |
| | | C (SEQ ID NO: 339) |
| | Ab-2 | EIVMTQSPATLSVSPGERATLSCRASQSVSSNLAWFQQKPGQAPRLLI |
| | | YGASTRATGIPARFSGSGSGTEFTLTISSLQPEDFAVYYCLQDNNFPPT |
| | | FGQGTKVDIKRADAAPTVSIFPPSSEQLTSGGASVVCFLNNFYPKDIN |
| | | VKWKIDGSERQNGVLNSWTDQDSKDSTYSMSSTLTLTKDEYERHNS |
| | | YTCEATHKTSTSPIVKSFNRNEC (SEQ ID NO: 2780) |
| | Ab-3 | EIVMTQSPATLSVSPGERATLSCRASQSVSSNLAWFQQKPGQAPRLLI |
| | | YGASTRATGIPARFSGSGSGTEFTLTISSLQPEDFAVYYCLQDNNFPPT |
| | | FGQGTKVDIKRADAAPTVSIFPPSSEQLTSGGASVVCFLNNFYPKDIN |
| | | VKWKIDGSERQNGVLNSWTDQDSKDSTYSMSSTLTLTKDEYERHNS |
| | | YTCEATHKTSTSPIVKSFNRNEC (SEQ ID NO: 2780) |
| Heavy | Ab-1 | EVQLVQSGAEVKKPGESLKISCKGSGYSFTSYWIGWVRQMPGKGLE |
| Chain | | WMGIIYPGDADARYSPSFQGQVTISADKSISTAYLQWSSLKASDTAM |
| | | YFCARRRQGIFGDALDFWGQGTLVTVSSASTKGPSVFPLAPSSKSTS |
| | | GGTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPCP |
| | | APELLGGPSVFLFPPKPKDTLMISRTPEVTCVVVDVSHEDPEVKFNW |
| | | YVDGVEVHNAKTKPCEEQYGSTYRCVSVLTVLHQDWLNGKEYKCK |
| | | VSNKALPAPIEKTISKAKGQPREPQVYTLPPSREEMTKNQVSLTCLVK |
| | | GFYPSDIAVEWESNGQPENNYKTTPPVLDSDGSFFLYSKLTVDKSRW |
| | | QQGNVFSCSVMHEALHNHYTQKSLSLSPGK (SEQ ID NO: 2774) |
| | Ab-1 w/ | MDMRVPAQLLGLLLLWLRGARCEVQLVQSGAEVKKPGESLKISCK |
| | leader | GSGYSFTSYWIGWVRQMPGKGLEWMGITYPGDADARYSPSFQGQVT |
| | sequence | ISADKSISTAYLQWSSLKASDTAMYFCARRRQGIFGDALDFWGQGTL |
| | | VTVSSASTKGPSVFPLAPSSKSTSGGTAALGCLVKDYFPEPVTVSWN |
| | | SGALTSGVHTFPAVLQSSGLYSLSSVVTVPSSSLGTQTYICNVNHKPS |
| | | NTKVDKKVEPKSCDKTHTCPPCPAPELLGGPSVFLFPPKPKDTLMISR |
| | | TPEVTCVVVDVSHEDPEVKFNWYVDGVEVHNAKTKPCEEQYGSTY |
| | | RCVSVLTVLHQDWLNGKEYKCKVSNKALPAPIEKTISKAKGQPREP |
| | | QVYTLPPSREEMTKNQVSLTCLVKGFYPSDIAVEWESNGQPENNYK |
| | | TTPPVLDSDGSFFLYSKLTVDKSRWQQGNVFSCSVMHEALHNHYTQ |
| | | KSLSLSPGK (SEQ ID NO: 340) |
| | Ab-2 | EVQLVQSGAEVKKPGESLKISCKGSGYSFTSYWIGWVRQMPGKGLE |
| | | WMGIIYPGDADARYSPSFQGQVTISADKSISTAYLQWSSLKASDTAM |
| | | YFCARRRQGIFGDALDFWGQGTLVTVSSAKTTPPSVYPLAPGSAAQT |
| | | NSMVTLGCLVKGYFPEPVTVTWNSGSLSSGVHTFPAVLQSDLYTLSS |
| | | SVTVPSSPRPSETVTCNVAHPASSTKVDKKIVPRDCGCKPCICTVPEV |
| | | SSVFIFPPKPKDVLTITLTPKVTCVVVDISKDDPEVQFSWFVDDVEVH |
| | | TAQTQPREEQFNSTFRSVSELPIMHQDWLNGKEFKCRVNSAAFPAPI |
| | | EKTISKTKGRPKAPQVYTIPPPKEQMAKDKVSLTCMITDFFPEDITVE |
| | | WQWNGQPAENYKNTQPIMNTNGSYFVYSKLNVQKSNWEAGNTFT |
| | | CSVLHEGLHNHHTEKSLSHSPGK (SEQ ID NO: 2781) |
| | Ab-3 | EVQLVQSGAEVKKPGESLKISCKGSGYSFTSYWIGWVRQMPGKGLE |
| | | WMGIIYPGDADARYSPSFQGQVTISADKSISTAYLQWSSLKASDTAM |
| | | YFCARRRQGIFGDALDFWGQGTLVTVSSAKTTPPSVYPLAPGSAAQT |
| | | NSMVTLGCLVKGYFPEPVTVTWNSGSLSSGVHTFPAVLQSDLYTLSS |
| | | SVTVPSSTWPSETVTCNVAHPASSTKVDKKIVPRDCGCKPCICTVPEV |
| | | SSVFIFPPKPKDVLTITLTPKVTCVVVDISKDDPEVQFSWFVDDVEVH |
| | | TAQTQPREEQFGSTFRSVSELPIMHQDWLNGKEFKCRVNSAAFPAPI |
| | | EKTISKTKGRPKAPQVYTIPPPKEQMAKDKVSLTCMITDFFPEDITVE |
| | | WQWNGQPAENYKNTQPIMDTDGSYFVYSKLNVQKSNWEAGNTFT |
| | | CSVLHEGLHNHHTEKSLSHSPGK (SEQ ID NO: 2779) |

In some embodiments, an anti-TREM2 antibody comprises a light chain having an amino acid sequence according to any one of SEQ ID NOS:2777, 339, and 2780, or any amino acid sequence that contains one or more, e.g., one, two, three, four or more amino acid substitutions (e.g., conservative amino acid substitutions), deletions or insertions of no more than five, four, three, two, or one amino acids to any one of SEQ ID NOS:2777, 339, and 2780. Such substitutions, deletions, and insertions would retain significant anti-TREM2 binding activity. In these and other embodiments, an anti-TREM2 antibody comprises a heavy chain having an amino acid sequence according to any one of SEQ ID NOS:2774, 340, 2779, and 2781, or any amino acid sequence that contains one or more, e.g., one, two, three, four or more amino acid substitutions (e.g., conservative amino acid substitutions), deletions or insertions of no more than five, four, three, two, or one amino acids to any one of SEQ ID NOS:2774, 340, 2779, and 2781. Such substitutions, deletions, and insertions would retain significant anti-TREM2 binding activity.

In some embodiments, an anti-TREM2 antibody comprises a light chain having an amino acid sequence according to SEQ ID NO:2777, and a heavy chain variable region having an amino acid sequence according to SEQ ID NO:2774. In some embodiments, an anti-TREM2 antibody comprises a light chain having an amino acid sequence according to SEQ ID NO:339, and a heavy chain variable region having an amino acid sequence according to SEQ ID NO:340. In some embodiments, an anti-TREM2 antibody comprises a light chain having an amino acid sequence according to SEQ ID NO:2780, and a heavy chain variable region having an amino acid sequence according to SEQ ID NO:2781. In some embodiments, an anti-TREM2 antibody comprises a light chain having an amino acid sequence according to SEQ ID NO:2780, and a heavy chain variable region having an amino acid sequence according to SEQ ID NO:2779.

### Antibody Constant Domains and Engineered Constant Regions

In some embodiments, any of the antigen binding agents, can have a constant domain on the light chain and/or the heavy chain of any origin. The term “constant region” as used herein refers to all domains of an antibody other than the variable region. The constant domain can be that of rodent, primate or other mammals. In some embodiments, the constant domain is of human origin. Accordingly, in some embodiments, any of the antigen binding agents described herein can have a human constant region, some of which are described above.

In some embodiments, a human constant region is, for example, a human light chain constant region or a human constant heavy chain region.

The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (x) or human lambda (λ) constant domain.

The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the antibody heavy chains.

In some embodiments, the human light chain constant region comprises a human kappa or human lambda constant region. In some embodiments, the antigen binding agents based on any light chain variable region or CDRs of a light chain variable region described herein includes a human light chain constant region, such as a kappa or lambda constant region sequences, which are found in all five antibody isotypes. Examples of human immunoglobulin light chain constant region sequences are shown in the following TABLE EN1.

TABLE EN1
Exemplary Human Immunoglobulin Light Chain Constant Regions

| Designation | CL Domain Amino Acid Sequence |
|---|---|
| Human lambda v1 | GQPKANPTVTLFPPSSEELQANKATLVCLISDFYPGAVTVAWKAD |
| | GSPVKAGVETTKPSKQSNNKYAASSYLSLTPEQWKSHRSYSCQVT |
| | HEGSTVEKTVAPTECS (SEQ ID NO: 191) |
| Human lambda v2 | GQPKAAPSVTLFPPSSEELQANKATLVCLISDFYPGAVTVAWKADS |
| | SPVKAGVETTTPSKQSNNKYAASSYLSLTPEQWKSHRSYSCQVTH |
| | EGSTVEKTVAPTECS (SEQ ID NO: 192) |
| Human lambda v3 | QPKAAPSVTLFPPSSEELQANKATLVCLISDFYPGAVTVAWKADSS |
| | PVKAGVETTTPSKQSNNKYAASSYLSLTPEQWKSHRSYSCQVTHE |
| | GSTVEKTVAPTECS (SEQ ID NO: 193) |
| Human lambda v4 | GQPKAAPSVTLFPPSSEELQANKATLVCLISDFYPGAVTVAWKADS |
| | SPVKAGVETTTPSKQSNNKYAASSYLSLTPEQWKSHKSYSCQVTH |
| | EGSTVEKTVAPTECS (SEQ ID NO: 194) |
| Human lambda v5 | GQPKAAPSVTLFPPSSEELQANKATLVCLVSDFYPGAVTVAWKAD |
| | GSPVKVGVETTKPSKQSNNKYAASSYLSLTPEQWKSHRSYSCRVT |
| | HEGSTVEKTVAPAECS (SEQ ID NO: 195) |
| Human kappa v1 | TVAAPSVFIFPPSDEQLKSGTASVVCLLNNFYPREAKVQWKVDNA |
| | LQSGNSQESVTEQDSKDSTYSLSSTLTLSKADYEKHKVYACEVTH |
| | QGLSSPVTKSFNRGEC (SEQ ID NO: 196) |
| Human kappa v2 | RTVAAPSVFIFPPSDEQLKSGTASVVCLLNNFYPREAKVQWKVDN |
| | ALQSGNSQESVTEQDSKDSTYSLSSTLTLSKADYEKHKVYACEVT |
| | HQGLSSPVTKSFNRGEC (SEQ ID NO: 197) |

In some embodiments, a human constant region comprises at least one or all of the following: a human CH1, human Hinge, human CH2, and CH3 domain. In some embodiments, the heavy chain constant region comprises an Fc region, where the Fc portion is a human IgG₁, IgG₂, IgG₃, IgG₄ or IgM isotype. The term “Fe region” refers to the C-terminal region of an immunoglobulin heavy chain which may be generated by papain digestion of an intact antibody. The Fc region of an immunoglobulin generally comprises two constant domains, a CH2 domain and a CH3 domain, and optionally comprises a CH4 domain. In certain embodiments, the Fc region is an Fc region from an IgG1, IgG2, IgG3, or IgG4 immunoglobulin. In some embodiments, the Fc region comprises CH2 and CH3 domains from a human IgG1 or human IgG2 immunoglobulin. The Fc region may retain effector function, such as C1q binding, complement-dependent cytotoxicity (CDC), Fc receptor binding, antibody-dependent cell-mediated cytotoxicity (ADCC), and phagocytosis. In other embodiments, the Fc region may be modified to reduce or eliminate effector function as described in further detail below.

In some embodiments, the antigen binding agents based on any heavy chain variable region or CDRs of a heavy chain variable region described herein includes a human heavy chain constant region., for example a human constant region comprising at least one or all of a human CH1, human Hinge, human CH2, and CH3 domain. In some embodiments, the antigen binding agents based on any heavy chain variable region or CDRs of a heavy chain variable region described herein includes an Fc region, where the Fc region is a human IgG₁, IgG₂, IgG₃, IgG₄ or IgM isotype. Examples of human IgG1, IgG2, and IgG4 heavy chain constant region sequences are shown below in TABLE EN2.

TABLE EN2
Exemplary Human Immunoglobulin Heavy Chain Constant Regions

| Ig isotype | Heavy Chain Constant Region Amino Acid Sequence |
|---|---|
| Human IgG1z | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKV |
| | DKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVV |
| | SVLTVLHQDWLNGKEYKCKVSNKALPAPIEKTISKAKGQPREPQVY |
| | TLPPSREEMTKNQVSLTCLVKGFYPSDIAVEWESNGQPENNYKTTP |
| | PVLDSDGSFFLYSKLTVDKSRWQQGNVFSCSVMHEALHNHYTQKS |
| | LSLSPGK (SESQ ID NO: 198) |
| Human IgG1za | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKV |
| | DKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVV |
| | SVLTVLHQDWLNGKEYKCKVSNKALPAPIEKTISKAKGQPREPQVY |
| | TLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSL |
| | SLSPGK (SESQ ID NO: 199) |
| Human IgG1f | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKV |
| | DKRVEPKSCDKTHTCPPCPAPELLGGPSVFLFPPKPKDTLMISRTPEV |
| | TCVVVDVSHEDPEVKFNWYVDGVEVHNAKTKPREEQYNSTYRVV |
| | SVLTVLHQDWLNGKEYKCKVSNKALPAPIEKTISKAKGQPREPQVY |
| | TLPPSREEMTKNQVSLTCLVKGFYPSDIAVEWESNGQPENNYKTTP |
| | PVLDSDGSFFLYSKLTVDKSRWQQGNVFSCSVMHEALHNHYTQKS |
| | LSLSPGK (SESQ ID NO: 200) |
| Human IgG1fa | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKV |
| | DKRVEPKSCDKTHTCPPCPAPELLGGPSVFLFPPKPKDTLMISRTPEV |
| | TCVVVDVSHEDPEVKFNWYVDGVEVHNAKTKPREEQYNSTYRVV |
| | SVLTVLHQDWLNGKEYKCKVSNKALPAPIEKTISKAKGQPREPQVY |
| | TLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSL |
| | SLSPGK (SESQ ID NO: 201) |
| Human IgG1z | ASTKGPSVFPLAPSSKSTSGGTAALGCLVKDYFPEPVTVSWNSGALT |
| aglycosylated | SGVHTFPAVLQSSGLYSLSSVVTVPSSSLGTQTYICNVNHKPSNTKV |
| v1 | DKKVEPKSCDKTHTCPPCPAPELLGGPSVFLFPPKPKDTLMISRTPEV |
| | TCVVVDVSHEDPEVKFNWYVDGVEVHNAKTKPREEQYGSTYRVV |
| | SVLTVLHQDWLNGKEYKCKVSNKALPAPIEKTISKAKGQPREPQVY |
| | TLPPSREEMTKNQVSLTCLVKGFYPSDIAVEWESNGQPENNYKTTP |
| | PVLDSDGSFFLYSKLTVDKSRWQQGNVFSCSVMHEALHNHYTQKS |
| | LSLSPGK (SESQ ID NO: 202) |
| Human IgG1z | ASTKGPSVFPLAPSSKSTSGGTAALGCLVKDYFPEPVTVSWNSGALT |
| aglycosylated v2 | SGVHTFPAVLQSSGLYSLSSVVTVPSSSLGTQTYICNVNHKPSNTKV |
| | DKKVEPKSCDKTHTCPPCPAPELLGGPSVFLFPPKPKDTLMISRTPEV |
| | TCVVVDVSHEDPEVKFNWYVDGVEVHNAKTKPCEEQYGSTYRCV |
| | SVLTVLHQDWLNGKEYKCKVSNKALPAPIEKTISKAKGQPREPQVY |
| | TLPPSREEMTKNQVSLTCLVKGFYPSDIAVEWESNGQPENNYKTTP |
| | PVLD SDGSFFLYSKLTVDKSRWQQGNVFSCSVMHEALHNHYTQKS |
| | LSLSPGK (SESQ ID NO: 203) |
| Human IgG2 | ASTKGPSVFPLAPCSRSTSESTAALGCLVKDYFPEPVTVSWNSGALT |
| | SGVHTFPAVLQSSGLYSLSSVVTVPSSNFGTQTYTCNVDHKPSNTK |
| | VDKTVERKCCVECPPCPAPPVAGPSVFLFPPKPKDTLMISRTPEVTC |
| | VVVDVSHEDPEVQFNWYVDGVEVHNAKTKPREEQFNSTFRVVSVL |
| | TVVHQDWLNGKEYKCKVSNKGLPAPIEKTISKTKGQPREPQVYTLP |
| | PSREEMTKNQVSLTCLVKGFYPSDIAVEWESNGQPENNYKTTPPML |
| | DSDGSFFLYSKLTVDKSRWQQGNVFSCSVMHEALHNHYTQKSLSL |
| | SPGK (SESQ ID NO: 204) |
| Human IgG4 | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKV |
| | DKKVEPKSCDKTHTCPPCPAPELLGGPSVFLFPPKPKDTLMISRTPEV |
| | TCVVVDVSHEDPEVKFNWYVDGVEVHNAKTKPCEEQYGSTYRCV |
| | SVLTVLHQDWLNGKEYKCKVSNKALPAPIEKTISKAKGQPREPQVY |
| | TLPPSREEMTKNQVSLTCLVKGFYPSDIAVEWESNGQPENNYKTTP |
| | PVLDSDGSFFLYSKLTVDKSRWQQGNVFSCSVMHEALHNHYTQKS |
| | LSLSPGK (SESQ ID NO: 205) |

In some embodiments, the heavy chain constant region, particularly the Fc region, is an engineered heavy chain constant region. In some embodiments, the antigen binding proteins, e.g. monoclonal antibodies, comprise one or more amino acid substitutions in the Fc region to enhance effector function, including ADCC activity, CDC activity, ADCP activity, and/or the clearance or half-life of the antigen binding protein. Exemplary amino acid substitutions (according to EU numbering scheme) that can enhance effector function include, but are not limited to, E233L, L234I, L234Y, L235S, G236A, S239D, F243L, F243V, P2471, D280H, K290S, K290E, K290N, K290Y, R292P, E294L, Y296W, S298A, S298D, S298V, S298G, S298T, T299A, Y300L, V3051, Q31 iM, K326A, K326E, K326W, A330S, A330L, A330M, A330F, 1332E, D333A, E333S, E333A, K334A, K334V, A339D, A339Q, P396L, or combinations of any of the foregoing.

In some embodiments, the TREM2 antigen binding proteins (e.g. monoclonal antibodies) comprise one or more amino acid substitutions in a heavy chain constant region to reduce effector function. Exemplary amino acid substitutions (according to EU numbering scheme) that can reduce effector function include, but are not limited to, C220S, C226S, C229S, E233P, L234A, L234V, V234A, L234F, L235A, L235E, G237A, P238S, S267E, H268Q, N297A, N297G, N297Q, V309L, E318A, L328F, A330S, A331S, P331S or combinations of any of the foregoing.

In some embodiments, the TREM2 agonist antigen binding proteins comprise one or more amino acid substitutions that affect the level or type of glycosylation of the binding proteins. Glycosylation can contribute to the effector function of antibodies, particularly IgG1 antibodies. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. 0-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

In some embodiments, glycosylation of the TREM2 agonist antigen binding proteins described herein is increased by adding one or more glycosylation sites, e.g., to the Fc region of the binding protein. Addition of glycosylation sites to the antigen binding protein can be conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antigen binding protein amino acid sequence may be altered through changes at the DNA level, particularly by mutating the DNA encoding the target polypeptide at preselected bases such that codons are generated that will translate into the desired amino acids.

The invention also encompasses production of TREM2 antigen binding protein molecules with altered carbohydrate structure resulting in altered effector activity, including antigen binding proteins with absent or reduced fucosylation that exhibit improved ADCC activity. Various methods are known in the art to reduce or eliminate fucosylation. For example, ADCC effector activity is mediated by binding of the antibody molecule to the FcγRIII receptor, which has been shown to be dependent on the carbohydrate structure of the N-linked glycosylation at the N297 residue of the CH2 domain. Non-fucosylated antibodies bind this receptor with increased affinity and trigger FcγRIII-mediated effector functions more efficiently than native, fucosylated antibodies. For example, recombinant production of non-fucosylated antibody in CHO cells in which the alpha-1,6-fucosyl transferase enzyme has been knocked out results in antibody with 100-fold increased ADCC activity (see Yamane-Ohnuki et al., Biotechnol Bioeng. 87(5):614-22, 2004). Similar effects can be accomplished through decreasing the activity of alpha-1,6-fucosyl transferase enzyme or other enzymes in the fucosylation pathway, e.g., through siRNA or antisense RNA treatment, engineering cell lines to knockout the enzyme(s), or culturing with selective glycosylation inhibitors (see Rothman et al., Mol Immunol. 26(12):1113-23, 1989). Some host cell strains, e.g. Lec13 or rat hybridoma YB2/0 cell line naturally produce antibodies with lower fucosylation levels (see Shields et al., J Biol Chem. 277(30):26733-40, 2002 and Shinkawa et al., J Biol Chem. 278(5):3466-73, 2003). An increase in the level of bisected carbohydrate, e.g. through recombinantly producing antibody in cells that overexpress GnTIII enzyme, has also been determined to increase ADCC activity (see Umana et al., Nat Biotechnol. 17(2):176-80, 1999).

In other embodiments, glycosylation of the TREM2 agonist antigen binding proteins described herein is decreased or eliminated by removing one or more glycosylation sites, e.g., from the Fc region of the binding protein. In some embodiments, the TREM2 agonist antigen binding protein is an aglycosylated human monoclonal antibody, e.g. an aglycosylated human IgG1 monoclonal antibody. Amino acid substitutions that eliminate or alter N-linked glycosylation sites can reduce or eliminate N-linked glycosylation of the antigen binding protein. In certain embodiments, the TREM2 agonist antigen binding proteins described herein comprise a mutation at position N297 (according to EU numbering scheme), such as N297Q, N297A, or N297G. In some embodiments, the TREM2 agonist antigen binding proteins of the invention comprise an Fc region from a human IgG1 antibody with a mutation at position N297. In one particular embodiment, the TREM2 agonist antigen binding proteins of the invention comprise an Fc region from a human IgG1 antibody with a N297G mutation. For instance, in some embodiments, the TREM2 agonist antigen binding proteins of the invention comprise a heavy chain constant region comprising the sequence of SEQ ID NO:202.

To improve the stability of molecules comprising a N297 mutation, the Fc region of the TREM2 agonist antigen binding proteins may be further engineered. For instance, in some embodiments, one or more amino acids in the Fc region are substituted with cysteine to promote disulfide bond formation in the dimeric state. Residues corresponding to V259, A287, R292, V302, L306, V323, or 1332 (according to EU numbering scheme) of an IgG1 Fc region may thus be substituted with cysteine. Preferably, specific pairs of residues are substituted with cysteine such that they preferentially form a disulfide bond with each other, thus limiting or preventing disulfide bond scrambling. Preferred pairs include, but are not limited to, A287C and L306C, V259C and L306C, R292C and V302C, and V323C and 1332C1n certain embodiments, the TREM2 agonist antigen binding proteins described herein comprise an Fc region from a human IgG1 antibody with mutations R292C and V302C1n such embodiments, the Fc region may also comprise a N297 mutation, such as a N297G mutation. In some embodiments, the TREM2 agonist antigen binding proteins of the invention comprise a heavy chain constant region comprising the sequence of SEQ ID NO:203.

Modifications to the hinge region and/or CH1 domain of the heavy chain and/or the constant region of the light chain of the TREM2 agonist antigen binding proteins (e.g. monoclonal antibodies) of the invention can be made to reduce or eliminate disulfide heterogeneity. Structural hetereogeneity of IgG2 antibodies has been observed where the disulfide bonds in the hinge and CH1 regions of IgG2 antibodies can be shuffled to create different structural disulfide isoforms (IgG2A, IgG2B, and IgG2A-B), which can have different levels of activity. See, e.g., Dillon et al., J. Biol. Chem., Vol. 283: 16206-16215; Martinez et al., Biochemistry, Vol. 47: 7496-7508, 2008; and White et al., Cancer Cell, Vol. 27: 138-148, 2015. Amino acid substitutions can be made in the hinge region, CH1 domain, and/or light chain constant region to promote the formation of a single disulfide isoform or lock the antigen binding protein (e.g. monoclonal antibody) into a particular disulfide isoform (e.g. IgG2A or IgG2B). Such mutations are described in WO 2009/036209 and White et al., Cancer Cell, Vol. 27: 138-148, 2015, both of which are hereby incorporated by reference in its entirety, and include C131S, C219S, and C220S (according to EU numbering scheme) mutations in the heavy chain and a C214S (according to EU numbering scheme) mutation in the light chain. In certain embodiments, the TREM2 agonist antigen binding proteins of the invention are human IgG2 anti-TREM2 agonist antibodies. In some such embodiments, the TREM2 agonist antibodies comprise a C131S mutation (according to the EU numbering scheme) in their heavy chains. In other embodiments, the TREM2 agonist antibodies comprise a C214S mutation (according to the EU numbering scheme) in their light chains and a C220S mutation (according to the EU numbering scheme) in their heavy chains. In still other embodiments, the TREM2 agonist antibodies comprise a C214S mutation (according to the EU numbering scheme) in their light chains and a C219S mutation (according to the EU numbering scheme) in their heavy chains.

In other embodiments, the TREM2 agonist antigen binding proteins of the invention are anti-TREM2 agonist antibodies comprising a CH1 region and hinge region from a human IgG2 antibody and an Fc region from a human IgG1 antibody. The unique arrangement of the disulfide bonds in the hinge region of IgG2 antibodies has been reported to impart enhanced stimulatory activity for certain anticancer antibodies (White et al., Cancer Cell, Vol. 27: 138-148, 2015). This enhanced activity could be transferred to IgG1-type antibodies by exchanging the CH1 and hinge regions of the IgG1 antibody for those in the IgG2 antibody (White et al., 2015). The IgG2 hinge region includes the amino acid sequence ERKCCVECPPCP (SEQ ID NO:206). The amino acid sequence of the CH1 and hinge regions from a human IgG2 antibody may comprise the following amino acid sequence:

(SEQ ID NO: 207)
ASTKGPSVFP LAPCSRSTSE STAALGCLVK DYFPEPVTVS
WNSGALTSGV HTFPAVLQSS GLYSLSSVVT VPSSNFGTQT
YTCNVDHKPS NTKVDKTVER KCCVECPPCP.

In some embodiments, the antigen binding agents based on any heavy chain variable region or CThus, in some embodiments, the anti-TREM2 agonist antibodies comprise the sequence of SEQ ID NO:207 in combination with an Fc region from a human IgG1 antibody. In such embodiments, the anti-TREM2 antibodies can comprise one or more of the mutations described above to lock the anti-TREM2 antibodies into a particular disulfide isoform. For instance, in one embodiment, the anti-TREM2 antibody comprises a CH1 region and hinge region from a human IgG2 antibody and an Fc region from a human IgG1 antibody and comprises a C131S mutation (according to the EU numbering scheme) in its heavy chain. In another embodiment, the anti-TREM2 antibody comprises a CH1 region and hinge region from a human IgG2 antibody and an Fc region from a human IgG1 antibody and comprises a C214S mutation (according to the EU numbering scheme) in its light chain and a C220S mutation (according to the EU numbering scheme) in its heavy chain. In yet another embodiment, the anti-TREM2 antibody comprises a CH1 region and hinge region from a human IgG2 antibody and an Fc region from a human IgG1 antibody and comprises a C214S mutation (according to the EU numbering scheme) in its light chain and a C219S mutation (according to the EU numbering scheme) in its heavy chain.

In embodiments in which the anti-TREM2 antibodies comprise a CH1 region and hinge region from a human IgG2 antibody and an Fc region from a human IgG1 antibody, the anti-TREM2 antibodies may comprise any of the mutations in the Fc region described above to modulate the glycosylation of the antibodies. For instance, the human IgG1 Fc region of such anti-TREM2 antibodies may comprise a mutation at amino acid position N297 (according to the EU numbering scheme) in its heavy chain. In one particular embodiment, the N297 mutation is a N297G mutation. In certain embodiments, the Fc region may further comprise R292C and V302C mutations (according to the EU numbering scheme) in its heavy chain.

In certain embodiments, the anti-TREM2 antibodies of the invention comprise a CH1 region and hinge region from a human IgG2 antibody and an Fc region from a human IgG1 antibody, wherein the Fc region comprises the amino acid sequence of:

(SEQ ID NO: 281)
APELLGGPSVFLFPPKPKDTLMISRTPEVTCVVVDVSHEDPEVKFNWYVD
GVEVHNAKTKPREEQYGSTYRVVSVLTVLHQDWLNGKEYKCKVSNKALPA
PIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHE
ALHNHYTQKSLSLSPGK.

In other embodiments, the anti-TREM2 antibodies of the invention comprise a CH1 region and hinge region from a human IgG2 antibody and an Fc region from a human IgG1 antibody, wherein the Fc region comprises the amino acid sequence of:

(SEQ ID NO: 282)
APELLGGPSVFLFPPKPKDTLMISRTPEVTCVVVDVSHEDPEVKFNWYVD
GVEVHNAKTKPCEEQYGSTYRCVSVLTVLHQDWLNGKEYKCKVSNKALPA
PIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHE
ALHNHYTQKSLSLSPGK.

Modifications of the TREM2 agonist antigen binding proteins of the invention to increase serum half-life also may desirable, for example, by incorporation of or addition of a salvage receptor binding epitope (e.g., by mutation of the appropriate region or by incorporating the epitope into a peptide tag that is then fused to the antigen binding protein at either end or in the middle, e.g., by DNA or peptide synthesis; see, e.g., WO96/32478) or adding molecules such as PEG or other water soluble polymers, including polysaccharide polymers. The salvage receptor binding epitope preferably constitutes a region wherein any one or more amino acid residues from one or two loops of an Fc region are transferred to an analogous position in the antigen binding protein. Even more preferably, three or more residues from one or two loops of the Fc region are transferred. Still more preferred, the epitope is taken from the CH2 domain of the Fc region (e.g., an IgG Fc region) and transferred to the CH1, CH3, or VH region, or more than one such region, of the antigen binding protein. Alternatively, the epitope is taken from the CH2 domain of the Fc region and transferred to the CL region or VL region, or both, of the antigen binding protein. See International applications WO 97/34631 and WO 96/32478 for a description of Fc variants and their interaction with the salvage receptor.

### Antibody Fragments

In some embodiments, the antigen binding agent can be a fragment of the antibody of the present disclosure, including portions of a full length antibody, and includes the antigen binding or variable region. Exemplary antibody fragments include Fab, Fab′, F(ab′)₂ and Fv fragments. In some embodiments, proteolytic digestion with papain produces two identical antigen binding fragments, the Fab′ fragment, each with a single antigen binding site. In some embodiments, proteolytic digestion with pepsin yields an F(ab′)₂ fragment that has two antigen binding fragments which are capable of cross-linking antigen, and a residual pFc′ fragment. In some embodiments, antibody fragments are produced directly in recombinant host-cells, for example host cells that that have a polynucleotide encoding an antigen binding agent described herein. For example, Fab, Fv and scFv antibody fragments can all be expressed in and secreted from E. coli, thus allowing the straightforward production of large amounts of these fragments. Anti-TREM2 antibody fragments can also be isolated from the antibody phage libraries as discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host-cell culture. Production of Fab and F(ab′)₂ antibody fragments with increased in vivo half-lives are described in U.S. Pat. No. 5,869,046. In other embodiments, the antibody of choice is a single chain Fv fragment (scFv). See WO 93/16185; U.S. Pat. Nos. 5,571,894, 5,587,458. Accordingly, other types of fragments can include diabodies, linear antibodies, single-chain antibodies, and multispecific antibodies formed from antibody fragments. In some embodiments, the antibody fragments are functional in that they retain the desired antigen binding properties, e.g., specific binding to TREM2, activation of TREM2 activities, and the like as described herein.

### Bispecific Antibodies

In some embodiments, the TREM2 binding protein is a bispecific antibody that binds to a TREM2 protein of the present disclosure and a second antigen. In some embodiments, bispecific antibodies of the present disclosure bind to one or more amino acid residues of human TREM2 (SEQ ID NO: 1), or amino acid residues on a TREM2 protein corresponding to amino acid residues of SEQ ID NO: 1. In some embodiments, any of the TREM2 binding proteins described herein can be used to prepare the bispecific antibody.

In some embodiments, bispecific antibodies of the present disclosure recognize a first antigen and a second antigen. In some embodiments, the first antigen is human TREM2 or a naturally occurring variant thereof. In some embodiments, the second antigen is DAP12, or other proteins or ligand that interact with TREM2. In some embodiments, the second antigen is (a) an antigen facilitating transport across the blood-brain-barrier; (b) an antigen facilitating transport across the blood-brain-barrier, for example transferrin receptor (TR), insulin receptor (HIR), insulin-like growth factor receptor (IGFR), low-density lipoprotein receptor related proteins 1 and 2 (LPR-1 and 2), diphtheria toxin receptor, CRM 197, a llama single domain antibody, TMEM 30(A), a protein transduction domain, TAT, Syn-B, penetratin, a poly-arginine peptide, an angiopep peptide, and ANG1005; (c) a disease-causing protein selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein A1, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides; and (d) ligands and/or proteins expressed on immune cells, wherein the ligands and/or proteins selected from CD40, OX40, ICOS, CD28, CD137/4-1BB, CD27, GITR, PD-L1, CTLA-4, PD-L2, PD-1, B7-H3, B7-H4, HVEM, BTLA, KIR, GAL9, TIM3, A2AR, LAG-3, and phosphatidylserine; and (e) a protein, lipid, polysaccharide, or glycolipid expressed on one or more tumor cells and any combination thereof.

Methods for making bispecific antibodies are known in the art. Traditional production of full-length bispecific antibodies is based on the coexpression of two immunoglobulin heavy-chain/light chain pairs, where the two chains have different specificities. Millstein et al., Nature, 305:537-539 (1983). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829 and in Traunecker et al., EMBO J., 10:3655-3659 (1991).

In some embodiments, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some embodiments, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only half of the bispecific molecules provides for an easy way of separation. This approach is disclosed in WO 94/04690. For further details of generating bispecific antibodies, see, for example, Suresh et al., Methods in Enzymology 121: 210 (1986); and Garber, Nature Reviews Drug Discovery 13, 799-801 (2014).

In some embodiments, the bispecific antibody can be prepared as described in WO 96/27011 or U.S. Pat. No. 5,731,168. In these embodiments, the interface between a pair of antibody molecules can be engineered to maximize the percentage of heterodimers which are recovered from recombinant-cell culture. The preferred interface comprises at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chains(s) are created on the interface of the second antibody molecule by replacing large amino acid side chains with smaller ones (e.g., alanine or threonine). This provides a mechanism for increasing the yield of the heterodimer over other unwanted end-products such as homodimers.

In some embodiments, bispecific antibody can be prepared Techniques for generating bispecific antibodies from antibody fragments have been described in for example, Brennan et al., Science, 1985, 229:81, which describe proteolytic cleavage of intact antibodies to generate F(ab′)2 fragments, which are then reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-TNB derivative to form the bispecific antibody. The bispecific antibodies produced can be used as agents for the selective immobilization of enzyme.

Various techniques for making and isolating bivalent antibody fragments directly from recombinant-cell culture have also been described. For example, bivalent heterodimers have been produced using leucine zippers. Kostelny et al., Immunol., 1992, 148(5):1547-1553. The “diabody” technology described by Hollinger et al., Proc. Nat'l Acad. Sci. USA, 1993, 90: 6444-6448, provides an alternative mechanism for making bispecific/bivalent antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific/bivalent antibody fragments by the use of single-chain Fv (sFv) dimers (see, e.g., Gruber et al., Immunol, 152:5368 (1994).

### Single Chain Antibodies

In some embodiments, the TREM2 binding protein is a single chain antibody, e.g., single chain Fv (sFv or scFv) antibodies, in which a variable heavy and a variable light chain are joined together (directly or through a peptide linker) to form a continuous polypeptide. A single-chain Fv” or “sFv” antibody fragments comprise the VH and VL domains of an antibody, where these domains are present in a single polypeptide chain. Generally, the Fv polypeptide further comprises a polypeptide linker between the VH and VL domains which enables the sFv to form the desired structure for antigen binding. For a review of sFv, see Pluckthun in The Pharmacology of Monoclonal Antibodies, Vol. 113, pp. 269-315, Rosenburg and Moore, eds., Springer-Verlag, New York (1994). Any of the TREM2 binding agents described herein can be used to prepare a single chain antibody.

In some embodiments, single chain antibody can be prepared by phage display methods, where the antigen binding domain is expressed as a single polypeptide and screened for specific binding activity. Alternatively, the single chain antibody can be prepared by cloning the heavy and light chains from a cell, typically a hybridoma cell line expressing a desired antibody. Generally, a linker peptide, typically from 10 to 25 amino acids in length is used to link the heavy and light chains. The linker can be glycine, serine, and/or threonine rich to impart flexibility and solubility to the single chain antibody. Specific methods for generating single chain antibodies are described in, for example, Loffler et al., 2000, Blood 95(6):2098-103; Worn and Pluckthun, 2001, J Mol Biol. 305, 989-1010; Pluckthun, In The Pharmacology of Monoclonal Antibodies, Vol. 113, pp. 269-315, Rosenburg and Moore, eds., Springer-Verlag, New York (1994); U.S. Pat. Nos. 5,840,301; 5,844,093; and 5,892,020; all of which are incorporated herein by reference.

### Multivalent Antibodies

In some embodiments, the anti-TREM2 antibody is a multivalent antibody, which may be internalized (and/or catabolized) faster than a bivalent antibody by a cell expressing an antigen to which the antibodies bind. In some embodiments, the anti-TREM2 antibodies of the present disclosure or antibody fragments thereof can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. A preferred dimerization domain comprises an Fc region or a hinge region. In this scenario, the antibody will comprise an Fc region and three or more antigen binding sites amino-terminal to the Fc region. The preferred multivalent antibody herein contains three to about eight, but preferably four, antigen binding sites. The multivalent antibody contains at least one polypeptide chain (and preferably two polypeptide chains), wherein the polypeptide chain or chains comprise two or more variable domains. For instance, the polypeptide chain or chains may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. Similarly, the polypeptide chain or chains may comprise VH-CH1-flexible linker- VH-CH1-Fc region chain; or VH-CH1-VH-CH1-FC region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

Multivalent antibodies may recognize the TREM2 antigen as well as without limitation additional antigens A beta peptide, antigen or an alpha synuclein protein antigen or, Tau protein antigen or, TDP-43 protein antigen or, prion protein antigen or, huntingtin protein antigen, or RAN, translation Products antigen, including the DiPeptide Repeats, (DPRs peptides) composed of glycine-alanine (GA), glycine-proline (GP), glycine-arginine (GR), proline-alanine (PA), or proline-arginine (PR), Insulin receptor, insulin like growth factor receptor. Transferrin receptor or any other antigen that facilitate antibody transfer across the blood brain barrier.

### Polynucleotides Encoding TREM12 Antibodies

In another aspect, the present disclosure provides polynucleotides encoding the antibodies or antigen binding regions of the described herein. In particular, the polynucleotides are isolated polynucleotides. The polynucleotides may be operatively linked to one or more heterologous control sequences that control gene expression to create a recombinant polynucleotide capable of expressing the polypeptide of interest. Expression constructs containing a heterologous polynucleotide encoding the relevant polypeptide or protein can be introduced into appropriate host cells to express the corresponding polypeptide.

As will be appreciated by those in the art, due to the degeneracy of the genetic code, where the same amino acids are encoded by alternative or synonymous codons, an extremely large number of nucleic acids can be made, all of which encode the CDRs, variable regions, and heavy and light chains or other components of the antigen binding proteins described herein. Thus, having identified a particular amino acid sequence, those skilled in the art could make any number of different nucleic acids, by simply modifying the sequence of one or more codons in a way which does not change the amino acid sequence of the encoded protein. In this regard, the present disclosure includes each and every possible variation of polynucleotides that encode the polypeptides disclosed herein.

An “isolated nucleic acid,” which is used interchangeably herein with “isolated polynucleotide,” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the nucleic acids are substantially free from contaminating endogenous material.

In some embodiments, the polynucleotide encodes a CDR L1, CDR L2 and CDR L3 of a light chain variable region described herein. In some embodiments, the polynucleotide encodes a CDR H1, CDR H2 and CDR H3 of a heavy chain variable region described herein.

In some embodiments, the polynucleotide encodes a CDR L1, CDR L2 and CDR L3 of a light chain variable region and a CDR H1, CDR H2 and CDR H3 of a heavy chain variable region described herein.

In some embodiments, the polynucleotide encodes a light chain variable region VL having at least 80%, 85%, 90%, 91%, 92%, 93%, 94%, 95%, 96%, 97%, 98%, 99% or greater sequence identity to the amino acid sequence of a variable light chain disclosed herein.

In some embodiments, the polynucleotide encodes a heavy chain variable region VH having at least 80%, 85%, 90%, 91%, 92%, 93%, 94%, 95%, 96%, 97%, 98%, 99% or greater sequence identity to the amino acid sequence of a variable heavy chain disclosed herein.

In some embodiments, the polynucleotides herein may be manipulated in a variety of ways to provide for expression of the encoded polypeptide. In some embodiments, the polynucleotide is operably linked to control sequences, including among others, transcription promoters, leader sequences, transcription enhancers, ribosome binding or entry sites, termination sequences, and polyadenylation sequences for expression of the polynucleotide and/or corresponding polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., Molecular Cloning: A Laboratory Manual, 3rd Ed., Cold Spring Harbor Laboratory Press (2001); and Current Protocols in Molecular Biology, Ausubel. F. ed., Greene Pub. Associates (1998), updates to 2013.

In some embodiments, variants of the antigen binding proteins, including the variants described herein, can be prepared by site-specific mutagenesis of nucleotides in the DNA encoding the polypeptide, using cassette or PCR mutagenesis or other techniques well known in the art, to produce DNA encoding the variant, and thereafter expressing the recombinant DNA in cell culture as outlined herein. However, antigen binding proteins comprising variant CDRs having up to about 100-150 residues may be prepared by in vitro synthesis using established techniques. The variants typically exhibit the same qualitative biological activity as the naturally occurring analogue, e.g., binding to antigen. Such variants include, for example, deletions and/or insertions and/or substitutions of residues within the amino acid sequences of the antigen binding proteins. Any combination of deletion, insertion, and substitution is made to arrive at the final construct, provided that the final construct possesses the desired characteristics. The amino acid changes also may alter post-translational processes of the antigen binding protein, such as changing the number or position of glycosylation sites. In some embodiments, antigen binding protein variants are prepared with the intent to modify those amino acid residues which are directly involved in epitope binding. In other embodiments, modification of residues which are not directly involved in epitope binding or residues not involved in epitope binding in any way, is desirable, for purposes discussed herein. Mutagenesis within any of the CDR regions, framework regions, and/or constant regions is contemplated. Covariance analysis techniques can be employed by the skilled artisan to design useful modifications in the amino acid sequence of the antigen binding protein. See, e.g., Choulier, et al., Proteins 41:475-484, 2000; Demarest et al., J. Mol. Biol., 2004, 335:41-48; Hugo et al., Protein Engineering, 2003, 16(5):381-86; Aurora et al., US Patent Publication No. 2008/0318207 A1; Glaser et al., US Patent Publication No. 2009/0048122 A1; Urech et al., WO 2008/110348 A1; Borras et al., WO 2009/000099 A2. Such modifications determined by covariance analysis can improve potency, pharmacokinetic, pharmacodynamic, and/or manufacturability characteristics of an antigen binding protein.

In another aspect, the present invention also provides vectors comprising one or more nucleic acids or polynucleotides encoding one or more components of the antigen binding proteins describe herein (e.g. variable regions, light chains, and heavy chains). As used herein, the term “vector” refers to any molecule or entity (e.g., nucleic acid, plasmid, bacteriophage or virus) used to transfer protein coding information into a host cell. Examples of vectors include, but are not limited to, plasmids, viral vectors, non-episomal mammalian vectors and expression vectors, for example, recombinant expression vectors. The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids. Exemplary expression vectors include, among others, vectors based on T7 or T71ac promoters (pACY: Novagen; pET); vectors based on Baculovirus promoters (e.g., pBAC); vectors based on Ef1-α and HTLV promoters (e.g., pFUSE2; Invitrogen, CA, USA); vectors based on CMV enhancer and human ferritin light chain gene promoters (e.g., pFUSE: Invitrogen, CA, USA); vectors based on CMV promoters (e.g, pFLAG: Sigma, USA); and vectors based on dihydrofolate reductase promoters (e.g., pEASE: Amgen, USA). Various vectors can be used for transient or stable expression of the polypeptides of interest.

### Host Cells

In another aspect, the polynucleotide encoding the antigen binding proteins described herein (e.g. variable regions, light chains, and heavy chains) is operatively linked to one or more control sequences for expression of the polypeptide in the host cell. Accordingly, in a further aspect, the present disclosure provides a host cell comprising one or more expression vectors encoding the components of the TREM2 agonist antigen binding proteins described herein.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Cantida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Host cells for the expression of glycosylated antigen binding proteins can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antigen binding proteins from such cells has become routine procedure. Mammalian cell lines available as hosts for expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA, 1980, 77: 4216); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod., 1980, 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB8065); mouse mammary tumor (MMT 060562, ATCC CCL51); TRI cells (Mather et al., Annals N.Y Acad. Sci., 1982, 383:44-68); MRC 5 cells or FS4 cells; mammalian myeloma cells, and a number of other cell lines. In certain embodiments, cell lines may be selected through determining which cell lines have high expression levels and constitutively produce antigen binding proteins with human TREM2 binding properties. In another embodiment, a cell line from the B cell lineage that does not make its own antibody but has a capacity to make and secrete a heterologous antibody can be selected. CHO cells are preferred host cells in some embodiments for expressing the TREM2 agonist antigen binding proteins of the invention.

In various embodiments, introduction and transformation of a host cell with a polynucleotide of the present disclosure, such as an expression vector for expressing an antigen binding protein, is accomplished by methods that including transfection, infection, calcium phosphate co-precipitation, electroporation, microinjection, lipofection, DEAE-dextran mediated transfection, or other known techniques. In some embodiments, the method selected can be guided by the type of host cell used. Suitable methods are described in, for example, Sambrook et al., 2001.

### Expression and Isolation

In some embodiments, the host cell comprising a polynucleotide encoding one or more components of the antigen binding proteins described herein (e.g. variable regions, light chains, and heavy chains) is used to express the antigen binding protein of interest. In some embodiments, a method for expressing the antigen binding protein comprises culturing the host cell in suitable media and conditions appropriate for expression of the protein of interest.

The type of media and culture conditions selected is based on the type of host cell. In some embodiments, exemplary media for mammalian host cells include, by way of example and not limitation, Ham's F10 (Sigma), Minimal Essential Medium (MEM, Sigma), RPMI-1640 (Sigma), and Dulbecco's Modified Eagle's Medium (DMEM, Sigma. In some embodiments, the media can be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. In some embodiments, culture conditions, such as temperature, pH, % CO₂, and the like, can use conditions available and known to the skilled artisan.

In some embodiments, the expressed antigen binding protein is isolate and/or purified from the host cell. In some embodiments in which the expressed protein in present in the media, the media containing the expressed protein is subject to isolation procedures. In some embodiments in which the antigen binding protein is produced intracellularly, the cells are subject to disruption, and as a first step, the particulate debris, either host cells or lysed fragments, is removed, for example, by centrifugation or ultrafiltration. Subsequently, the antigen binding protein can be isolated and further purified by various known techniques. Such isolation techniques include affinity chromatography with Protein-A Sepharose, size-exclusion chromatography, ion-exchange chromatography, high performance liquid chromatography, differential solubility, and the like (see, e.g., Fisher, Laboratory Techniques, In Biochemistry And Molecular Biology, Work and Burdon, eds., Elsevier (1980); Antibodies: A Laboratory Manual, Greenfield, E.A., ed., Cold Spring Harbor Laboratory Press, New York (2012); Coligan, et al., supra, sections 2.7.1-2.7.12 and sections 2.9.1-2.9.3; Barnes, et al., Purification of Immunoglobulin G (IgG), in Methods Mol. Biol., Vol. 10, pages 79-104, Humana Press (1992)).

In some embodiments, the isolated antibody can be further purified as measurable by: (1) weight of protein as determined using the Lowry method; (2) to a degree sufficient to obtain at least 15 residues of N-terminal or internal amino acid sequence by use of a spinning-cup sequencer; or (3) to homogeneity by SDS-PAGE under reducing or non-reducing conditions using Coomassie blue or, preferably, silver stain. The purified antibody can be 85% or greater, 90% or greater, 95% or greater, or at least 99% by weight as determined by the foregoing methods.

### Antibody Formulations

In certain embodiments, the invention provides a composition (e.g. a pharmaceutical composition) comprising one or a plurality of the TREM2 activating antibodies and TREM2 agonist antibodies and antigen binding proteins disclosed herein together with pharmaceutically acceptable diluents, carriers, excipients, solubilizers, emulsifiers, preservatives, and/or adjuvants. Pharmaceutical compositions of the invention include, but are not limited to, liquid, frozen, and lyophilized compositions. “Pharmaceutically-acceptable” refers to molecules, compounds, and compositions that are non-toxic to human recipients at the dosages and concentrations employed and/or do not produce allergic or adverse reactions when administered to humans. In some embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. Methods and suitable materials for formulating molecules for therapeutic use are known in the pharmaceutical arts, and are described, for example, in Remington's Pharmaceutical Sciences, 18th Ed., (A.R. Genrmo, ed.), 1990, Mack Publishing Company.

In some embodiments, the pharmaceutical composition of the invention comprises a standard pharmaceutical carrier, such as a sterile phosphate buffered saline solution, bacteriostatic water, and the like. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like.

Exemplary concentrations of the antigen binding proteins in the formulation may range from about 0.1 mg/ml to about 200 mg/ml or from about 0.1 mg/mL to about 50 mg/mL, or from about 0.5 mg/mL to about 25 mg/mL, or alternatively from about 2 mg/mL to about 10 mg/mL. An aqueous formulation of the antigen binding protein may be prepared in a pH-buffered solution, for example, at pH ranging from about 4.5 to about 6.5, or from about 4.8 to about 5.5, or alternatively about 5.0. Examples of buffers that are suitable for a pH within this range include acetate (e.g. sodium acetate), succinate (such as sodium succinate), gluconate, histidine, citrate and other organic acid buffers. The buffer concentration can be from about 1 mM to about 200 mM, or from about 10 mM to about 60 mM, depending, for example, on the buffer and the desired isotonicity of the formulation.

A tonicity agent, which may also stabilize the antigen binding protein, may be included in the formulation. Exemplary tonicity agents include polyols, such as mannitol, sucrose or trehalose. Preferably the aqueous formulation is isotonic, although hypertonic or hypotonic solutions may be suitable. Exemplary concentrations of the polyol in the formulation may range from about 1% to about 150% w/v.

A surfactant may also be added to the antigen binding protein formulation to reduce aggregation of the formulated antigen binding protein and/or minimize the formation of particulates in the formulation and/or reduce adsorption. Exemplary surfactants include nonionic surfactants such as polysorbates (e.g., polysorbate 20 or polysorbate 80) or poloxamers (e.g., poloxamer 188). Exemplary concentrations of surfactant may range from about 0.001% to about 0.5%, or from about 0.005% to about 0.2%, or alternatively from about 0.004% to about 0.01% w/v.

In one embodiment, the formulation contains the above-identified agents (i.e. antigen binding protein, buffer, polyol and surfactant) and is essentially free of one or more preservatives, such as benzyl alcohol, phenol, m-cresol, chlorobutanol and benzethonium chloride. In another embodiment, a preservative may be included in the formulation, e.g., at concentrations ranging from about 0.1% to about 2%, or alternatively from about 0.5% to about 1%. One or more other pharmaceutically acceptable carriers, excipients or stabilizers such as those described in REMINGTON'S PHARMACEUTICAL SCIENCES, 18th Edition, (A.R. Genrmo, ed.), 1990, Mack Publishing Company, may be included in the formulation provided that they do not adversely affect the desired characteristics of the formulation.

Therapeutic formulations of the antigen binding protein are prepared for storage by mixing the antigen binding protein having the desired degree of purity with optional physiologically acceptable carriers, excipients or stabilizers (Remington's Pharmaceutical Sciences, 18th Ed., (A.R. Genrmo, ed.), 1990, Mack Publishing Company), in the form of lyophilized formulations or aqueous solutions. Acceptable carriers, excipients, or stabilizers are nontoxic to recipients at the dosages and concentrations employed, and include buffers (e.g. phosphate, citrate, and other organic acids); antioxidants (e.g. ascorbic acid and methionine); preservatives (such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol; resorcinol, cyclohexanol, 3-pentanol, and m-cresol); low molecular weight (e.g. less than about 10 residues) polypeptides; proteins (such as serum albumin, gelatin, or immunoglobulins); hydrophilic polymers (e.g. polyvinylpyrrolidone); amino acids (e.g. glycine, glutamine, asparagine, histidine, arginine, or lysine); monosaccharides, disaccharides, and other carbohydrates including glucose, mannose, maltose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants, such as polysorbates (e.g. polysorbate 20 or polysorbate 80) or poloxamers (e.g. poloxamer 188); or polyethylene glycol (PEG).

In one embodiment, a suitable formulation of the claimed invention contains an isotonic buffer such as a phosphate, acetate, or TRIS buffer in combination with a tonicity agent, such as a polyol, sorbitol, sucrose or sodium chloride, which tonicifies and stabilizes. One example of such a tonicity agent is 5% sorbitol or sucrose. In addition, the formulation could optionally include a surfactant at 0.01% to 0.02% wt/vol, for example, to prevent aggregation or improve stability. The pH of the formulation may range from 4.5 to 6.5 or 4.5 to 5.5. Other exemplary descriptions of pharmaceutical formulations for antigen binding proteins may be found in US Patent Publication No. 2003/0113316 and U.S. Pat. No. 6,171,586, each of which is hereby incorporated by reference in its entirety.

Suspensions and crystal forms of antigen binding proteins are also contemplated. Methods to make suspensions and crystal forms are known to one of skill in the art.

The formulations to be used for in vivo administration must be sterile. The compositions of the invention may be sterilized by conventional, well-known sterilization techniques. For example, sterilization is readily accomplished by filtration through sterile filtration membranes. The resulting solutions may be packaged for use or filtered under aseptic conditions and lyophilized, the lyophilized preparation being combined with a sterile solution prior to administration.

The process of freeze-drying is often employed to stabilize polypeptides for long-term storage, particularly when the polypeptide is relatively unstable in liquid compositions. A lyophilization cycle is usually composed of three steps: freezing, primary drying, and secondary drying (see Williams and Polli, Journal of Parenteral Science and Technology, 1984, 38(2):48-59). In the freezing step, the solution is cooled until it is adequately frozen. Bulk water in the solution forms ice at this stage. The ice sublimes in the primary drying stage, which is conducted by reducing chamber pressure below the vapor pressure of the ice, using a vacuum. Finally, sorbed or bound water is removed at the secondary drying stage under reduced chamber pressure and an elevated shelf temperature. The process produces a material known as a lyophilized cake. Thereafter the cake can be reconstituted prior to use.

The standard reconstitution practice for lyophilized material is to add back a volume of pure water (typically equivalent to the volume removed during lyophilization), although dilute solutions of antibacterial agents are sometimes used in the production of pharmaceuticals for parenteral administration (see Chen, Drug Development and Industrial Pharmacy, Volume 18: 1311-1354, 1992).

Excipients have been noted in some cases to act as stabilizers for freeze-dried products (see Carpenter et al., Volume 74: 225-239, 1991). For example, known excipients include polyols (including mannitol, sorbitol and glycerol); sugars (including glucose and sucrose); and amino acids (including alanine, glycine and glutamic acid).

In addition, polyols and sugars are also often used to protect polypeptides from freezing and drying-induced damage and to enhance the stability during storage in the dried state. In general, sugars, in particular disaccharides, are effective in both the freeze-drying process and during storage. Other classes of molecules, including mono- and di-saccharides and polymers such as PVP, have also been reported as stabilizers of lyophilized products.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Sustained-release preparations may be prepared. Suitable examples of sustained-release preparations include semipermeable matrices of solid hydrophobic polymers containing the antigen binding protein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S bond formation through thio-disulfide interchange, stabilization may be achieved by modifying sulfhydryl residues, lyophilizing from acidic solutions, controlling moisture content, using appropriate additives, and developing specific polymer matrix compositions.

The formulations of the invention may be designed to be short-acting, fast-releasing, long-acting, or sustained-releasing. Thus, the pharmaceutical formulations may also be formulated for controlled release or for slow release.

Specific dosages may be adjusted depending on the disease, disorder, or condition to be treated, the age, body weight, general health conditions, sex, and diet of the subject, dose intervals, administration routes, excretion rate, and combinations of drugs.

The TREM2 agonist antigen binding proteins of the invention can be administered by any suitable means, including parenteral, subcutaneous, intraperitoneal, intrapulmonary, intrathecal, intracerebral, intracerebroventricular, and intranasal, and, if desired for local treatment, intralesional administration. Parenteral administration includes intravenous, intraarterial, intraperitoneal, intramuscular, intradermal or subcutaneous administration. In addition, the antigen binding protein is suitably administered by pulse infusion, particularly with declining doses of the antigen binding protein. Preferably, the dosing is given by injections, most preferably intravenous or subcutaneous injections, depending in part on whether the administration is brief or chronic. Other administration methods are contemplated, including topical, particularly transdermal, transmucosal, rectal, oral or local administration e.g. through a catheter placed close to the desired site. In certain embodiments, the TREM2 agonist antigen binding protein of the invention is administered intravenously or subcutaneously in a physiological solution at a dose ranging between 0.01 mg/kg to 100 mg/kg at a frequency ranging from daily to weekly to monthly (e.g. every day, every other day, every third day, or 2, 3, 4, 5, or 6 times per week), preferably a dose ranging from 0.1 to 45 mg/kg, 0.1 to 15 mg/kg or 0.1 to 10 mg/kg at a frequency of once per week, once every two weeks, or once a month.

In some embodiments, the anti-TREM2 antibody is administered to a human patient via an IV infusion. In some embodiments, an IV infusion of anti-TREM2 antibody is up to about 5 hours, up to about 4 hours, up to about 3 hours, up to about 2 hours, or up to about 60 minutes. In some embodiments, an IV infusion of anti-TREM2 antibody is from about 5 minutes to about 5 hours, from about 5 minutes to about 4 hours, from about 5 minutes to about 3 hours, from about 5 minutes to about 2 hours, or from about 5 minutes to about 60 minutes. In some embodiments, an IV infusion of anti-TREM2 antibody is about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, or about 90 minutes.

In some embodiments, anti-TREM2 antibody is administered to a human patient at a dose of up to about 200 mg/kg. In some embodiments, anti-TREM2 antibody is administered to a human patient at a dose of up to about 150 mg/kg. In some embodiments, anti-TREM2 antibody is administered to a human patient at a dose of up to about 100 mg/kg. In some embodiments, anti-TREM2 antibody is administered to a human patient at a dose of from about 1 mg/kg to about 100 mg/kg, from about 1 mg/kg to about 90 mg/kg, from about 1 mg/kg to about 80 mg/kg, from about 1 mg/kg to about 70 mg/kg, or from about 1 mg/kg to about 60 mg/kg. In some embodiments, anti-TREM2 antibody is administered to a human patient at a dose of about 1 mg/kg, about 2 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, or about 60 mg/kg.

In some embodiments, anti-TREM2 antibody is administered to a human patient once daily. In some embodiments, anti-TREM2 antibody is administered to a human patient 1, 2, 3 or 4 times weekly. In some embodiments, anti-TREM2 antibody is administered to a human patient 1, 2, 3 or 4 times monthly. In some embodiments, anti-TREM2 antibody is administered to a human patient once every 1, 2, 3, or 4 weeks. In some embodiments, anti-TREM2 antibody is administered to a human patient once every 2 days, 3 days, 4 days, 5 days, 6 days, 7 days, 8 days, 9 days, 10 days, or 14 days. In some embodiments, anti-TREM2 antibody is administered to a human patient once weekly.

In some embodiments, the present invention provides a liquid formulation, comprising anti-TREM2 antibody at a concentration of up to about 300 mg/mL. In some embodiments, the present invention provides a liquid formulation, comprising anti-TREM2 antibody at a concentration of up to about 250 mg/mL. In some embodiments, the present invention provides a liquid formulation, comprising anti-TREM2 antibody at a concentration of up to about 200 mg/mL. In some embodiments, the present invention provides a liquid formulation, comprising anti-TREM2 antibody at a concentration of up to about 150 mg/mL. In some embodiments, the present invention provides a liquid formulation, comprising anti-TREM2 antibody at a concentration of about 300 mg/mL, about 250 mg/mL, about 200 mg/mL, about 180 mg/mL, about 170 mg/mL, about 160 mg/mL, about 150 mg/mL, about 140 mg/mL, about 130 mg/mL, about 120 mg/mL, about 110 mg/mL, or about 100 mg/mL. In some embodiments, the present invention provides a liquid formulation, comprising anti-TREM2 antibody at a concentration of about 140 mg/mL. In some embodiments, a method of the invention comprises administering to a human patient a liquid formulation as described herein. In some embodiments, a method of the invention comprises administering to a human patient a liquid formulation, comprising anti-TREM2 antibody at a concentration of about 140 mg/mL.

The TREM2 agonist antigen binding proteins described herein (e.g. anti-TREM2 agonist monoclonal antibodies and binding fragments thereof) are useful for preventing, treating, or ameliorating a condition associated with TREM2 deficiency or loss of biological function of TREM2 in a patient in need thereof. As used herein, the term “treating” or “treatment” is an intervention performed with the intention of preventing the development or altering the pathology of a disorder. Accordingly, “treatment” refers to both therapeutic treatment and prophylactic or preventative measures. Patients in need of treatment include those already diagnosed with or suffering from the disorder or condition as well as those in which the disorder or condition is to be prevented, such as patients who are at risk of developing the disorder or condition based on, for example, genetic markers. “Treatment” includes any indicia of success in the amelioration of an injury, pathology or condition, including any objective or subjective parameter such as abatement, remission, diminishing of symptoms, or making the injury, pathology or condition more tolerable to the patient, slowing in the rate of degeneration or decline, making the final point of degeneration less debilitating, or improving a patient's physical or mental well-being. The treatment or amelioration of symptoms can be based on objective or subjective parameters, including the results of a physical examination, self-reporting by a patient, cognitive tests, motor function tests, neuropsychiatric exams, and/or a psychiatric evaluation.

### III. Small Molecule TREM2 Agonists

In some embodiments, the agonist of TREM2 is a small molecule agonist of TREM2.

In some embodiments, the agonist of TREM2 is a lipid ligand of TREM2. In some embodiments, the lipid ligand of TREM2 is selected from 1-palmitoyl-2-(5′-oxo-valeroyl)-sn-glycero-3-phosphocholine (POVPC), 2-Arachidonoylglycerol (2-AG), 7-ketocholesterol (7-KC), 24(S)hydroxycholesterol (240HC), 25(S)hydroxycholesterol (250HC), 27-hydroxycholesterol (270HC), Acyl Carnitine (AC), alkylacylglycerophosphocholine (PAF), a-galactosylceramide (KRN7000), Bis(monoacylglycero)phosphate (BMP), Cardiolipin (CL), Ceramide, Ceramide-1-phosphate (CIP), Cholesteryl ester (CE), Cholesterol phosphate (CP), Diacylglycerol 34: 1 (DG 34: 1), Diacylglycerol 38:4 (DG 38:4), Diacylglycerol pyrophosphate (DGPP), Dihyrdoceramide (DhCer), Dihydrosphingomyelin (DhSM), Ether phosphatidylcholine (PCe), Free cholesterol (FC), Galactosylceramide (GalCer), Galactosylsphingosine (GalSo), Ganglioside GM1, Ganglioside GM3, Glucosylsphingosine (GlcSo), Hank's Balanced Salt Solution (HBSS), Kdo2-Lipid A (KLA), Lactosylceramide (LacCer), lysoalkylacylglycerophosphocholine (LPAF), Lysophosphatidic acid (LPA), Lysophosphatidylcholine (LPC), Lysophosphatidylethanolamine (LPE), Lysophosphatidylglycerol (LPG), Lysophosphatidylinositol (LPI), Lysosphingomyelin (LSM), Lysophosphatidylserine (LPS), N-Acyl-phosphatidylethanolamine (NAPE), N-Acyl-Serine (NSer), Oxidized phosphatidylcholine (oxPC), Palmitic-acid-9-hydroxy-stearic-acid (PAHSA), Phosphatidylethanolamine (PE), Phosphatidylethanol (PEtOH), Phosphatidic acid (PA), Phosphatidylcholine (PC), Phosphatidylglycerol (PG), Phosphatidylinositol (PI), Phosphatidylserine (PS), Sphinganine, Sphinganine-1-phosphate (SalP), Sphingomyelin (SM), Sphingosine, Sphingosine-1-phosphate (SolP), or Sulfatide, or a salt thereof.

In some embodiments, the agonist of TREM2 is a lipopolysaccharide.

In some embodiments, the agonist of TREM2 is a small molecule disclosed in PCT Application Publication WO2019/079529, which is incorporated by reference herein in its entirety. In some embodiments, the agonist of TREM2 is Tyrphostin AG 538, AC1NS458, IN1040, Butein, Okanin, AGL 2263, GB19, GB16, GB20, GB17, GB18, GB21, GB22, GB27, GB44, GB42, GB2, 4,4′-Dihydroxychalcone, or 3,4-Dihydroxybenzophenone, or a derivative or salt of any of the aforementioned.

In some embodiments, the agonist of TREM2 is a small molecule identified by a method disclosed in PCT Application Publication WO2019/079529. In some embodiments, the small molecule agonist of TREM2 is identified by applying the small molecule compound to a host cell expressing TREM2 and tyrosine kinase binding protein (TYROBP), wherein the host cell has a synthetic sequence comprising an NFAT-response element and a nucleotide sequence encoding a reporter, and measuring a signal emitted by the reporter.

In some embodiments, the agonist of TREM2 is a small molecule disclosed in PCT Application Publication WO2021/226135 or WO2021/226629.

In some embodiments, the agonist of TREM2 is a TREM2 agonist compound comprising a bicyclic core. In some embodiments, the bicyclic core is a 10-membered heteroaryl core. In some embodiments, the TREM2 agonist compound comprises a 10-membered heteroaryl core, comprising 1-4 nitrogen atoms as part of the core ring structure. In some embodiments, the TREM2 agonist compound comprises a 10-membered heteroaryl core, comprising 3 or 4 substituent groups.

### IV. Other TREM2 Agonists

In some embodiments, the agonist of TREM2 is heat shock protein 60 (HSP60).

In some embodiments, the agonist of TREM2 is apopoliprotein E (ApoE).

### V. Pharmaceutically Acceptable Compositions

In certain embodiments, a TREM2 activating antibody or small molecule disclosed herein is formulated as a composition for administration to a patient in need of such composition.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

In other embodiments, pharmaceutically acceptable compositions of this invention are formulated for intravenous (IV) administration.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

All features of each of the aspects of the disclosure apply to all other aspects mutatis mutantis. Each of the references referred to herein, including but not limited to patents, patent applications and journal articles, is incorporated by reference herein as though fully set forth in its entirety.

In order that the disclosure described herein may be more fully understood, the following examples are set forth. It should be understood that these examples are for illustrative purposes only and are not to be construed as limiting this disclosure in any manner.

### EXAMPLES

### General Procedures

### Microglial Differentiation Protocol

Pluripotent stem cell (PSC) differentiation is induced with mTeSR Custom medium (STEMCELLTechnologies®) containing 80 ng/mL BMP4. At day 4, cells are induced with 25 ng/mL basic fibroblast growth factor, 100 ng/mL stemcell factor (SCF), and 80 ng/mL vascular endothelial growth factor in StemPro-34 SFM (with 2 mM GutaMAX, Life Technologies). Two days later, the medium is supplemented with 50 ng/mL SCF, 50 ng/mL IL-3, 5 ng/mL thrombopoietin, 50 ng/mL macrophage CSF (M-CSF) and 50 ng/mL Flt31, and from day 14 with 50 ng/mL M-CSF, 50 ng/mL Flt31, and 25 ng/mL GMCSF. Between days 25 and 50, CD14+ or CD14+CX3CR1+ progenitors are isolated and plated onto tissue culture-treated dishes or Thermanox plastic coverslips (all from Thermo Fisher Scientific) in Microglial Medium (RPMI-1640 [Life Technologies] with 2 mM GlutaMAX-I, 10 ng/mL GM-CSF, and 100 ng/mL IL-34). Medium is replenished every 3-4 days for at least 2 weeks.

Generation of Induced Pluripotent Stem Cells (iPSCs) from Primary Healthy, Adrenomyeloneuropathy (AMN) and Cerebral Childhood ALD (cALD)Fibroblasts

Fibroblast cell cultures from healthy individuals (AGO1439, male 3 days old), Adrenomyeloneuropathy (AMN) (GM 07530, male 26 years old) and cALD (GM04934, male 7 years old with VLCFA abnormality and clinical X-ALD disease) patients are obtained. A control human IPSC ATCC-DYRO100 cell line is also obtained. Fibroblasts are cultured in DMEM with 10% FBS, 2 mM L-glutamine and 1% penicillin/streptomycin at 37° C. with 5% CO₂.

Fibroblast cells seeded at 0.2×106 cells/well of a 6-well plate in fibroblast medium (DMEM+10% FBS) are transduced with six lentiviral vectors designed to deliver human OCT4, SOX2, c-MYC, KLF4, Nanog and Lin28 cDNA sequences. On the next day, fresh fibroblast media is added to the cells 24 hours after transduction. At 48 hours after transduction the media is changed to half E8 medium and half fibroblast medium. When the cells reach about 60% confluence they are transferred to 10 cm Matrigel-coated plates (one well of a 6-well plate into one 10 cm dish) in E8 medium (StemCell Technologies) and media is replaced daily. Between day 15 and day 30 in culture, individual hiPS clones are manually picked using a Leica stereomicroscope. Each hiPS clone is expanded and characterized by immunofluorescence for the expression of Oct4 and Tra-1-60. IPSCs are cultured on a Matrigel (BD-Biosceicnes) coated plate in IPSC medium (mTeSR media from Stemcell technologies, Vancouver, Canada) and media is changed daily until cells are ready for passage.

### Microglia Characterization Assays and Methods

Microglia are analyzed using flow cytometry, immunohistochemistry and cell quantification procedures such as those disclosed by Masuda et al. (Masuda et al., Nature. 2019; 566: 388-394.)

### Monocyte Derived Macrophage Isolation Protocols

Monocyte derived macrophages (MDMs) are derived from PBMCs collected from patients with adrenomyeloneuropathy (AMN) or cALD and isolated by magnetic bead separation and differentiation into macrophages as reported in Jin, et al. J Vis Exp. 2016; (112): 54244. CD14+ monocytes are collected for use in the Examples described below.

### Example 1. The Effects of TREM2 Agonists on Microglia after VLCFA Challenge

### Conceptual Basis

Saturated very long chain fatty acids (VLCFAs; ≥C22:0) accumulate in the microglia of patients suffering from X-linked adrenoleukodystrophy (X-ALD, OMIM 300100), a severe hereditary neurodegenerative disease, due to peroxisomal impairment. Previous studies analysed the development of X-ALD in humans and gene knockout animal models. However, the toxic effect of VLCFA leading to severe symptoms with progressive and multifocal demyelination, adrenal insufficiency and inflammation still remains unclear. To understand the toxic effects of VLCFA in the brain, neural cells are exposed to VLCFA and the effects are analyzed. Oligodendrocytes and astrocytes challenged with docosanoic-(C22:0), tetracosanoic- (C24:0) and hexacosanoic acids (C26:0) die within 24 h. VLCFA-induced depolarization of mitochondria in situ and increased intracellular Ca21 level in all three brain cell types provides indications about the mechanism of toxicity of VLCFA. VLCFAs affect to the largest degree the myelin-producing oligodendrocytes. In isolated mitochondria, VLCFAs exert a detrimental effect by affecting the inner mitochondrial membrane and promoting the permeability transition. Without intending to be limited to any particular theory, it is reasonable to conclude that there is potent toxic activity of VLCFA due to dramatic cell physiological effects with mitochondrial dysfunction and Ca21 deregulation. This provides evidence for mitochondrial-based cell death mechanisms in neurodegenerative diseases with peroxisomal defects and subsequent VLCFA accumulation. Treatment with a TREM2 agonist rescues at least some of the deleterious effects of VLCFA accumulation in microglia, by preventing apoptosis and activating phagocytic mechanisms to clear myelin debris to lower inflammation in the vicinity of axonopathy.

### Experiments

Healthy patient-derived microglia are plated in 96 well microtiter plates. Each well also contains either a TREM2 agonist (eg. a TREM2 antibody agonist or TREM2 small molecule agonist) or a control compound (eg. an isotype control IgG as a control antibody or DMSO as a control small molecule). An exemplary method uses an immobilized TREM2 antibody agonist in the test wells at 10 pg/well and an isotype control plated in the control wells at 10 pg/well. Another exemplary method uses a solubilized TREM2 antibody agonist or small molecule agonist in the test wells. Cells are maintained in CSF1-containing culture medium (50 ng/mL) for 2 days prior to adding VLCFA (e.g., C26:0, C24:0, C22:0 added at 10-20 uM concentration to culture medium; Hein et al., Human Molecular Genetics. 2008; 17: 1750-176.). After 24 hrs of VLCFA challenge, wells are analyzed microscopically by immunohistochemical staining, e.g., by staining for Iba1, P2YR12 or other myeloid cell surface markers, staining for caspases, which indicate cell apoptosis, and for the number of viable cells as well as for cell morphology. Other microtiter wells can be treated with lysis buffer to collect mRNA for qPCR analysis for markers of myeloid phenotype, such as homeostatic or activated cell states (Keren-Shaul et al., Cell. 2017; 169: 1276-1290; Decskowska et al., Cell. 2018; 173:1073-1081.). Other microtiter wells can be analyzed for total cell death by measuring lactate dehydrogenase levels, a measure of cell lysis, from the culture supernatant (Hein et al., Human Molecular Genetics. 2008; 17: 1750-176.). This experiment can be modified to measure the kinetics of phenotypic transition following the pilot described above by analyzing changes over time and at various doses of VGL101 to understand the dose dependence of rescue. In addition, the Incucyte method of monitoring cell cultures can be used to monitor for morphological changes in real time.

The above experiments are also repeated using monocytes derived macrophages, in place of the microglia.

### Example 2. The Effects of TREM2 Agonists on Microglia with an ABCD1 Dysfunction

### Conceptual Basis

Microglia derived from patients suffering from cALD, such as by differentiating patient iPSC or patient monocytes, have transcriptional and biochemical signatures distinct from healthy donor-derived microglia, e.g., potentially enriched for disease associated microglia (DAM). Treatment of cALD patient-derived microglia with VLCFA in vitro will cause toxic and pro-inflammatory effects due to accumulation of fatty acids from dysfunctional peroxisome-mediated metabolism. cALD microglia may also autonomously accumulate VLCFAs without an extracellular challenge. Rescue of the cytotoxicity and inflammatory state by treating with TREM2 agonists during VLCFA challenge can be characterized through transcriptional and biochemical analyses.

### Experiments

iPSC or monocyte-derived microglia, derived from patients suffering from an ABCD1 dysfunction, such as cALD or AMN, are plated in 96 well microtiter plates. Each well also contains either a TREM2 agonist (eg. a TREM2 antibody agonist or TREM2 small molecule agonist) or a control compound (eg. an isotype control IgG as a control antibody or DMSO as a control small molecule). An exemplary method uses an immobilized TREM2 antibody agonist in the test wells at 10 pg/well and an isotype control plated in the control wells at 10 pg/well. Another exemplary method uses a solubilized TREM2 antibody agonist or small molecule agonist in the test wells. Cells are maintained in a CSF1-containing culture medium (50 ng/mL) for 2 days prior to adding VLCFA (e.g., C26:0, C24:0, C22:0 added at 10-20 uM concentration to culture medium; Hein et al., Human Molecular Genetics. 2008; 17: 1750-176.). Vehicle is added to select wells, in place of the VLCFA. After 24 hrs of challenge, wells are analyzed microscopically by immunohistochemical staining, e.g., by staining for Iba1, PYR12 or other myeloid cell surface markers, staining for caspases, which indicate cell apoptosis, and for the number of viable cells as well as for cell morphology. Other microtiter wells can be treated with lysis buffer to collect mRNA for qPCR analysis for markers of myeloid phenotype, such as homeostatic or activated cell states (Keren-Shaul et al., Cell. 2017; 169: 1276-1290; Decskowska et al., Cell. 2018; 173:1073-1081.). Other microtiter wells can be analyzed for total cell death by measuring lactate dehydrogenase levels, a measure of cell lysis, from the culture supernatant (Hein et al., Human Molecular Genetics. 2008; 17: 1750-176.). This experiment can be modified to measure the kinetics of phenotypic transition following the pilot described above by analyzing changes over time and at various doses of VLCFA or VGL101 to understand the dose dependence of rescue. In addition, the Incucyte method of monitoring cell cultures can be used to monitor for morphological changes in real time.

The above experiments are also repeated using monocytes derived macrophages, in place of the iPSC or microglia. The above experiments may also be repeated, wherein the VLCFA challenge is replaced with alternative additions known to induce increased accumulation of VLCFAs in cALD microglia. For example, myelin debris or lipids can be added to the wells in place of the VLCFA.

### Example 3. Neurofilament Light Chain as a Biomarker for Tracking x-ALD Treatment Efficacy

Monitoring of serum from patients with x-ALD for levels of neurofilament light chain (NfL) in order to select patients for treatment, and to monitor the efficacy of treatment will be done as follows. Serum is collected from patients at various time points as required for the use. Serum is stored in sample aliquots at −80° C. When ready for analysis, samples are thawed on ice. Measurement of NfL is determined using an assay run on a Simoa® HD-1 instrument (QUANTERIX) using a 2-step Assay Neat 2.0 protocol; 100 μl of sample or calibrator (diluent: Tris-buffered saline [TBS], 0.1% Tween 20, 1% milk powder, 400 μg/ml Heteroblock [Omega Biologicals, Bozeman, MT]), 25 μl conjugated beads (diluent: TBS, 0.1% Tween 20, 1% milk powder, 300 ug/ml Heteroblock), and 20 μl of mAB2:1 (0.1 pg/ml; diluent: TBS, 0.1% Tween 20, 1% milk powder, 300 ug/ml Heteroblock) are incubated for 47 cadences (1 cadence=45 seconds). After washing, 100 μl of streptavidin-conjugated b-galactosidase (150 μM; Quanterix) is added, followed by a 7-cadence incubation and a wash. Prior to reading, 25 μl resorufin b-D-galactopyranoside (QUANTERIX) is added. Calibrators (neat) and samples (serum: 1:4 dilution) are measured in duplicates. Bovine lyophilized NfL is obtained from UmanDiagnostics. Calibrators ranged from 0 to 2,000 μg/ml for serum and from 0 to 10,000 μg/ml for CSF measurements. Batch prepared calibrators are stored at −80° C. Final NfL levels measured by the above method are used to help both select patients to treat with an agonist of TREM2 and guide response to treatment with an agonist of TREM2.

1. A method of treating a disease or disorder caused by and/or associated with ATP-binding cassette transporter 1 (ABCD1) dysfunction in a human patient, the method comprising administering to the patient an effective amount of an agonist of triggering receptor expressed on myeloid cells 2 (TREM2).

2. The method of claim 1, wherein the disease or disorder is selected from X-linked adrenoleukodystrophy (x-ALD), Globoid cell leukodystrophy, Metachromatic leukodystrophy (MLD), Cerebral autosomal dominant arteriopathy with subcortical infarcts and leukoencephalopathy (CADASIL), Alexander disease, fragile X-associated tremor ataxia syndrome (FXTAS), adult-onset autosomal dominant leukodystrophy (ADLD), and X-linked Charcot-Marie-Tooth disease (CMTX).

3. The method of claim 1, wherein the disease or disorder is selected from Nasu-Hakola disease, Alzheimer's disease, frontotemporal dementia, multiple sclerosis, Guillain-Barre syndrome, amyotrophic lateral sclerosis (ALS), or Parkinson's disease; wherein the patient exhibits ABCD1 dysfunction, and/or has a mutation in a gene affecting the function of ABCD1.

4. The method of claim 1, wherein the disease or disorder is x-ALD.

5. The method of claim 1, wherein the disease or disorder is Addison disease wherein the patient has been found to have a mutation in one or more ABCD1 genes affecting ABCD1 function.

6. The method of claim 1, wherein the administration of the agonist of TREM2 increases microglia function in the patient.

7. The method of claim 1, wherein the agonist of TREM2 activates TREM2/DAP12 signaling in myeloid cells.

8. The method of claim 1, wherein the agonist of TREM2 activates, induces, promotes, stimulates, or otherwise increases one or more TREM2 activities selected from:
(a) TREM2 binding to DAP12; DAP12 binding to TREM2; TREM2 phosphorylation; DAP12 phosphorylation;
(b) PI3K activation;
(c) increased levels of soluble TREM2 (sTREM2);
(d) increased levels of soluble CSF1R (sCSF1R);
(e) increased expression of one or more anti-inflammatory mediators selected from the group consisting of IL-12p70, IL-4, IL-6, and IL-1β;
(f) reduced expression of one or more pro-inflammatory mediators selected from the group consisting of IFN-α4, IFN-b, IL-6, IL-12 p70, IL-12 p40, IL-1β, TNF, TNF-α, IL-1β, IL-8, CRP, TGF-beta members of the chemokine protein families, IL-20 family members, IL-33, LIF, IFN-gamma, OSM, CNTF, TGF-beta, GM-CSF, IL-11, IL-12, IL-17, IL-18, and CRP;
(g) increased expression of one or more chemokines selected from the group consisting of CCL2, CCL4, CXCL10, CCL3 and CST7;
(h) reduced expression of TNF-α, IL-6, or both; extracellular signal-regulated kinase (ERK) phosphorylation; increased expression of C-C chemokine receptor 7 (CCR7);
(i) induction of microglial cell chemotaxis toward CCL19 and CCL21 expressing cells;
(j) an increase, normalization, or both of the ability of bone marrow-derived dendritic cells to induce antigen-specific T-cell proliferation;
(k) induction of osteoclast production, increased rate of osteoclastogenesis, or both; increasing the survival and/or function of one or more of dendritic cells, macrophages, microglial cells, M1 macrophages and/or microglial cells, activated M1 macrophages and/or microglial cells, M2 macrophages and/or microglial cells, monocytes, osteoclasts, Langerhans cells of skin, and Kupffer cells;
(l) induction of one or more types of clearance selected from the group consisting of apoptotic neuron clearance, nerve tissue debris clearance, non-nerve tissue debris clearance, bacteria or other foreign body clearance, disease-causing protein clearance, disease-causing peptide clearance, and disease-causing nucleic acid clearance;
(m) induction of phagocytosis of one or more of apoptotic neurons, nerve tissue debris, non-nerve tissue debris, bacteria, other foreign bodies, disease-causing proteins, disease-causing peptides, or disease-causing nucleic acids; normalization of disrupted TREM2/DAP12-dependent gene expression;
(n) recruitment of Syk, ZAP70, or both to the TREM2/DAP12 complex; Syk phosphorylation; increased expression of CD83 and/or CD86 on dendritic cells, macrophages, monocytes, and/or microglia;
(o) reduced secretion of one or more inflammatory cytokines selected from the group consisting of TNF-α, IL-1β, IL-6, MCP-1, IFN-a4, IFN-b, IL-1β, IL-8, CRP, TGF-beta members of the chemokine protein families, IL-20 family members, IL-33, LIF, IFN-gamma, OSM, CNTF, TGF-beta, GM-CSF, IL-11, IL-12, IL-17, IL-18, and CRP;
(p) reduced expression of one or more inflammatory receptors; increasing phagocytosis by macrophages, dendritic cells, monocytes, and/or microglia under conditions of reduced levels of MCSF;
(q) decreasing phagocytosis by macrophages, dendritic cells, monocytes, and/or microglia in the presence of normal levels of MCSF; increasing activity of one or more TREM2-dependent genes;
(r) increased levels of one or more of CSF1, CSF2 and IL-34; or
(s) any combination thereof.

9. The method of claim 1, wherein the agonist of TREM2 is an antigen binding protein or an antibody, or an antigen-binding fragment thereof.

10. The method of claim 9, wherein the agonist of TREM2 is a monoclonal antibody, a humanized antibody, or a human antibody.

11-12. (canceled)

13. The method of claim 1, wherein the agonist of TREM2 is an antibody or antigen-binding fragment thereof that specifically binds to the polypeptide of SEQ ID NO: 1.

14. The method of claim 13, wherein the antibody or antigen-binding fragment thereof binds specifically to a polypeptide of amino acid residues 19-174 of SEQ ID NO: 1 or a polypeptide of amino acid residues 19-140 of SEQ ID NO: 1.

15. (canceled)

16. The method of claim 1, wherein the agonist of TREM2 is an antibody or antigen-binding fragment thereof comprising a light chain variable region having a CDRL1, CDRL2, and CDRL3 selected from Table EX1 and A10, and a heavy chain variable region having a CDRH1, CDRH2, and CDRH3 selected from Table EX2 and A11.

17. (canceled)

18. The method of claim 16, wherein the TREM2 agonist is an antibody or antigen binding fragment thereof comprising:
(a) a CDRL1, CDRL2, and CDRL3 comprising the sequence of SEQ ID NOs: 8, 22, and 35, respectively, and a CDRH1, CDRH2, and CDRH3 comprising the sequence of SEQ ID NOs: 77, 368, and 98, respectively;
(b) a CDRL1, CDRL2, and CDRL3 comprising the sequence of SEQ ID NOs: 16, 369, and 370, respectively, and a CDRH1, CDRH2, and CDRH3 comprising the sequence of SEQ ID NOs: 85, 371, and 107, respectively;
(c) a CDRL1, CDRL2, and CDRL3 comprising the sequence of SEQ ID NOs: 10, 23, and 372, respectively, and a CDRH1, CDRH2, and CDRH3 comprising the sequence of SEQ ID NOs: 81, 373, and 374, respectively; or
(d) a CDRL1, CDRL2, and CDRL3 comprising the sequence of SEQ ID NOs: 17, 29, and 44, respectively, and a CDRH1, CDRH2, and CDRH3 comprising the sequence of SEQ ID NOs: 86, 94, and 375, respectively.

19. The method of claim 1, wherein the agonist of TREM2 is an antibody or antigen-binding fragment thereof comprising a light chain variable region selected from Table EX1 or A14, and a heavy chain variable region selected from Table EX2 and A15.

20. (canceled)

21. The method of claim 19, wherein the antibody or antigen-binding fragment thereof comprises:
(a) a light chain variable region comprising the amino acid sequence of SEQ ID NO: 326 and a heavy chain variable region comprising the amino acid sequence of SEQ ID NO: 327;
(b) a light chain variable region comprising the amino acid sequence of SEQ ID NO: 328 and a heavy chain variable region comprising the amino acid sequence of SEQ ID NO: 329;
(c) a light chain variable region comprising the amino acid sequence of SEQ ID NO: 330 and a heavy chain variable region comprising the amino acid sequence of SEQ ID NO: 331; or
(d) a light chain variable region comprising the amino acid sequence of SEQ ID NO: 332 and a heavy chain variable region comprising the amino acid sequence of SEQ ID NO: 333.

22. The method of claim 1, wherein the agonist of TREM2 is a small molecule agonist of TREM2.

23. The method of claim 22, wherein the agonist of TREM2 is a lipid ligand of TREM2.

24. The method of claim 23, wherein the agonist of TREM2 is selected from 1-palmitoyl-2-(5′-oxo-valeroyl)-sn-glycero-3-phosphocholine (POVPC), 2-Arachidonoylglycerol (2-AG), 7-ketocholesterol (7-KC), 24(S)hydroxycholesterol (240HC), 25(S)hydroxycholesterol (250HC), 27-hydroxycholesterol (270HC), Acyl Carnitine (AC), alkylacylglycerophosphocholine (PAF), a-galactosylceramide (KRN7000), Bis(monoacylglycero)phosphate (BMP), Cardiolipin (CL), Ceramide, Ceramide-1-phosphate (CIP), Cholesteryl ester (CE), Cholesterol phosphate (CP), Diacylglycerol 34: 1 (DG 34: 1), Diacylglycerol 38:4 (DG 38:4), Diacylglycerol pyrophosphate (DGPP), Dihyrdoceramide (DhCer), Dihydrosphingomyelin (DhSM), Ether phosphatidylcholine (PCe), Free cholesterol (FC), Galactosylceramide (GalCer), Galactosylsphingosine (GalSo), Ganglioside GM1, Ganglioside GM3, Glucosylsphingosine (GlcSo), Hank's Balanced Salt Solution (HBSS), Kdo2-Lipid A (KLA), Lactosylceramide (LacCer), lysoalkylacylglycerophosphocholine (LPAF), Lysophosphatidic acid (LPA), Lysophosphatidylcholine (LPC), Lysophosphatidylethanolamine (LPE), Lysophosphatidylglycerol (LPG), Lysophosphatidylinositol (LPI), Lysosphingomyelin (LSM), Lysophosphatidylserine (LPS), N-Acyl-phosphatidylethanolamine (NAPE), N-Acyl-Serine (NSer), Oxidized phosphatidylcholine (oxPC), Palmitic-acid-9-hydroxy-stearic-acid (PAHSA), Phosphatidylethanolamine (PE), Phosphatidylethanol (PEtOH), Phosphatidic acid (PA), Phosphatidylcholine (PC), Phosphatidylglycerol (PG), Phosphatidylinositol (PI), Phosphatidylserine (PS), Sphinganine, Sphinganine-1-phosphate (SalP), Sphingomyelin (SM), Sphingosine, Sphingosine-1-phosphate (SolP), or Sulfatide, or a salt thereof.

25. (canceled)

26. The method of claim 22, wherein the agonist of TREM2 is selected from Tyrphostin AG 538, AC1NS458, IN1040, Butein, Okanin, AGL 2263, GB19, GB16, GB20, GB17, GB18, GB21, GB22, GB27, GB44, GB42, GB2, 4,4′-Dihydroxychalcone, or 3,4-Dihydroxybenzophenone, or a salt thereof.

27-28. (canceled)
